(19) United States
(12) Patent Application Publication
(10) Pub. No.: US 2026/0139223 A1
(43) Pub. Date: May 21, 2026

(54) GENE ACTIVATION AND INTERFERENCE TARGETS FOR EXHAUSTION/DYSFUNCTIONRESISTANT T CELL PRODUCTS AND USES THEREOF

(71) Applicants: The J. David Gladstone Institutes, a testamentary trust established under the Will of J. David Glads, San Francisco, CA (US); The Regents of the University of California, Oakland, CA (US)

(72) Inventors: Zachary Steinhart, San Francisco, CA (US); Alexander Marson, San Francisco, CA (US)

(21) Appl. No.: 19/120,196

(22) PCT Filed: Nov. 3, 2023
(86) PCT No.: PCT/US2023/036810

§ 371 Date: Apr. 10, 2025

Related U.S. Application Data
(60) Provisional application No. 63/422,243, filed on Nov. 3, 2022.

(51) Int. Cl. C12N 5/0783; A61K 35/17; A61K 40/11; A61K 40/42; A61P 35/00; C12N 5/10; C12N 9/22; C12N 15/113; C12N 15/85

(52) U.S. Cl. CPC C12N 5/0636; A61K 35/17; A61K 40/11; A61K 40/42; A61P 35/00; C12N 5/10; C12N 9/222; C12N 15/113; C12N 15/85; C12N 2310/20; C12N 2510/00; C12N 2830/002

(57) ABSTRACT

Provided herein are gene activation and interference targets for exhaustion/dysfunction-resistant t cell products and uses thereof.

### CROSS-REFERENCE TO RELATED APPLICATIONS

This application claims the benefit of U.S. Provisional Application No. 63/422,243, filed Nov. 3, 2022, the content of which is herein incorporated by reference in its entirety.

### BACKGROUND

Current T cell therapeutics for cancer are limited in scope and response rate due to limitations in T cell function, such as T cell exhaustion. T cell “exhaustion” is where repeated antigen exposure leads to a dysfunctional T cell state and incomplete tumor clearance. Exhausted T cells have limited proliferative, cytotoxic, and cytokine secretion capacities and have upregulated inhibitory immune checkpoint receptors.

### SUMMARY

Regulators that play a role in T cell exhaustion/dysfunction resistance are described herein as well as methods of using such regulators. Genome-wide CRISPR activation (CRISPRa) and CRISPR interference (CRISPRi) screens were performed in primary human T cells to identify genes that are upregulated or downregulated, respectively, to cause exhaustion/dysfunction resistance. These CRISPRa and CRISPRi screens discovered 37 and 69 genes (FDR<0.1), respectively, that when upregulated or downregulated caused exhaustion/dysfunction resistance.

Provided herein are modified T cells, lymphoid cells or myeloid cells comprising a modification of one of more genes selected from BATF, CUL3, CLASRP, CDKL3, EOMES, FOXL2NB, FOXO6, FOXO4, FOSB, FOSL1, FOXL2, GLIS3, GLI2, IRF4, IL2RA, INPP5D, IL2RB, ITPKA, ITPKB, IL10, IL9R, LIFR, LCK, LYN, NPM3, NQO1, PLEKHH3, PTEN, RFLNB, STAT5A, SHOC2, S100A13, TRAF3IP2. TMEM167B, TSR2, VAV1 or ZNF211, wherein the modification upregulates or increases expression of said gene in said modified T cells, lymphoid cells or myeloid cells as compared to a non-modified T cell, lymphoid cell or myeloid cell control. In one aspect, the modification is one or more deletion, substitution or insertion into one or more of said genes. One aspect provides an increase in expression by modification of one or more endogenous promoters of one or more of said genes. In aspect, the modified T cell, lymphoid cell or myeloid cell comprises one or more expression cassettes comprising a promoter operably linked to a nucleic acid segment comprising a coding region of any of said genes or a targeted knock-in of a cassette under the control of a selected endogenous promoter/gene regulatory element.

One aspect provides a modified T cell, lymphoid cell or myeloid cell comprising a modification of one of more of genes selected from CCNC, ELOB, ELOC, IGFL4, MED1, MED12, MED13. MN1, MED19, MEOX1, MOCS3, NQO2. PRDM14, SYCE1L, SYTL2, SRRD, SPIN1, UBE2E1, ARHGAP5. ARIH1, ARIH2, ATF7IP, ATF7IP2. ATXN7L3. BORCS8, CAND1, CASP3, CBFB, CDK13, CDKN2A, CFAP54, CISH, COPS7B, CPSF4L, CUL5, CUTA, DCUN1D3, DDA1, GABARAPL1, KDM5C, KIAA1551, KRTAP10-9, MADD, MAPKAP1, MED23, MED24, MYB, MYO5A, NFKBIA, PAK2, PDCL, PIK3R5, PTEN, RASA2. RNASE9, RNF7. RPS6KA1, SBNO2. SND1, ST8SIA5, TNIP1, TRIM56, TSPEAR. UBAP2L, UBE2F, UBE2L3, WDFY1, ZBTB21 or ZMYND8, wherein the modification downregulates or decreases expression of said gene in said modified T cell, lymphoid cell or myeloid cell as compared to a non-modified T cell, lymphoid cell or myeloid cell control. In one aspect, the modified T cell, lymphoid cell or myeloid cell comprises an exogenous inhibitory nucleic acid (e.g., RNAi, shRNA, siRNA).

Methods are described herein that involve in vivo or ex vivo modification of any of the genes listed in Table 1 or FIGS. 1-27 within at least one cell, such as a lymphoid or myeloid cell or a T-cell or combination thereof. For example, the modification can be one or more deletions, substitutions or insertions into one or more endogenous genomic sites of any of the genes listed in Table 1 or FIGS. 1-27. The modification can be reduction of expression or translation of any of the genes listed in Table 1 or FIGS. 1-27. The reduction of expression or translation can be by an inhibitory nucleic acid (e.g., RNAi, shRNA, siRNA or CRISPR (nuclease of epigenetic engineering). The modification can be increased expression of any of the genes listed in Table 1 or FIGS. 1-27. For example, the increased expression can be by modification of one or more promoters of any of the genes listed in Table 1 or FIGS. 1-27. The modification can be one or more CRISPR-mediated modifications or activations of any of the genes listed in Table 1 or FIGS. 1-27.

For example, genes discovered in the CRISPRa screen can be engineered for overexpression by cDNA transgene delivery, targeted genomic knock-in of a transgene, or promotor/epigenetic engineering at the endogenous locus. Conversely, genes discovered in the CRISPRi screen can be interfered with by gene knockout or promotor/epigenetic engineering. Additionally, protein products of the discovered genes can be activated or inhibited through biologics or small molecules to enhance T cell function, either post adoptive T cell therapy, as a standalone immunotherapy, or in combination with other immunotherapies such as bispecific T cell engagers or checkpoint blockade.

The methods also include administering at least one of the modified lymphoid cells, at least one of the modified myeloid cells, at least one of the modified T cells, or a combination thereof to a subject.

In some cases, the method can include incubating the at least one modified lymphoid cell, at least one modified myeloid cell, at least one modified T cell or a mixture thereof to form a population of modified cells. Such a population of modified cells can be administered to a subject. In some cases, the subject can have a disease or condition. For example, the disease or condition is an immune condition or cancer.

Also described are methods that involve contacting at least one test agent with test cells to provide a test assay mixture, and measuring: cellular proliferation of the test cells, cytokine release by the test cells, or a combination thereof, activation of the test cells; expression or activity of any of the genes listed in Table 1 or FIGS. 1-27 in the cells: or a combination thereof. The methods can also include comparing the measured results to control results. The control results can be results of the test cells measured without any of the test agents.

For example, the test cells can include lymphoid and/or myeloid cells. Examples of the test cells can include cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune (e.g., lymphoid and/or myeloid) cells, or a combination thereof. The results so measured can be compared to results of a control cell mixture that includes the T cells and test cells measured without any of the test agents.

### DRAWINGS

FIGS. 1A-1C provide an overview of genome-wide CRISPRi/a screens for regulators of CD8+ CAR T cell fitness in an exhaustion/dysfunction model and demonstrates that chronic signaling of HA GD2 CAR drives CD8+ T cells to exhaustion/dysfunction.

FIG. 2 provides CRISPRi (loss-of-function) exhaustion/dysfunction screen results.

FIG. 3 demonstrates that genes encoding signal transduction proteins in IL-2 signaling pathway are depleted in both CARs.

FIG. 4 shows CRISPRi screen results—IL2R/STAT5 pathway genes play a role in both CAR Ts.

FIG. 5 shows CRISPRi screen results—several gene knockdowns were positively enriched in the HA-GD2 CAR condition.

FIG. 6 shows CRISPRi screen results—demonstrates that mediator complex genes negatively regulate T cell fitness in the HA-GD2 CAR condition.

FIG. 7 demonstrates that PI3K—MTORC2 pathway member knockout causes increased fitness specifically in the HA GD2 CAR condition.

FIG. 8 depicts CRISPRa (gain-of-function) exhaustion screen results.

FIG. 9 shows CRISPRa screen results—several genes were positively enriched in the HA-GD2 CAR condition.

FIG. 10 shows CRISPRa screen results—activation of IL2R and STAT5 were positively enriched in the HA-GD2 CAR condition.

FIG. 11 demonstrates that overexpression of several transcription factors increases fitness HA GD2 CAR T cells.

FIG. 12 shows CRISPRa screen results—selecting transcription factor hits for further characterization in arrayed format.

FIG. 13 depicts arrayed CRISPRa perturbations for screen validation and deeper phenotyping.

FIG. 14 demonstrates that proliferation assay validates CRISPRa hits.

FIG. 15 demonstrates that FOXL2NB CRISPRa greatly enhances in vitro target cell killing with HA GD2 CAR T cells.

FIG. 16 shows that FOXL2NB CRISPRa increases cytokine production and GranzymeB staining.

FIG. 17 FOXL2NB shares a bidirectional promoter with FOXL2.

FIG. 18 demonstrates that cDNA overexpression reveals FOXL2 as a driver of the exhaustion resistance phenotype.

FIG. 19 demonstrates that FOXL2 cDNA overexpression also increases killing in a CD19 CAR repetitive stimulation model.

FIG. 20 shows gene expression changes caused by FOXL2/FOXL2NB CRISPRa in non-exhausted T cells.

FIG. 21 demonstrates that FOXL2 maintains high IL2RA surface expression.

FIG. 22 provides NALM6-GD2 Killing across E:T ratios data.

FIG. 23 provides 143B GD2 CAR Killing data.

FIG. 24 provides 143B GD2 CAR Killing—E:T=3 data.

FIG. 25 provides 143B GD2 CAR Killing—E:T=3—Hours—90 data.

FIG. 26 provides CRISPRa—CD19 CAR and CRISPRa—HA-GD2 CAR data.

FIG. 27 provides NALM6-GD2 Killing with GD2 CAR T cells.

### DETAILED DESCRIPTION

Provided herein is the use of genome wide CRISPR activation (CRISPRa) and interference (CRISPRi) screens in primary human CD8⁺ T cells to discover genes that are upregulated or downregulated, respectively, to cause exhaustion/dysfunction resistance. To model exhaustion/dysfunction, CD8⁺ T cells were engineered to express the high-affinity anti-GD2 chimeric antigen receptor (GD2-CAR), which due to chronic CAR signaling mimics chronic antigen stimulation and drives cells to an exhausted state (Lynn et al. Nature. 2019 December; 576(7786):293-300. doi: 10.1038/s41586-019-1805-z. Epub 2019 Dec. 4; PMID:31802004). Pooled lentiviral genome-wide CRISPRa and CRISPRi perturbations were applied to GD2-CAR T cells and directly compared them with a gold-standard anti-CD19 CAR T cell non-exhausted control population. By measuring the abundance of sgRNA expressing cells over time in both GD2-CAR and CD19-CAR T cells in parallel, perturbations were determined that allowed for enhanced proliferation and survival, specifically in the exhausted context. The CRISPRa and CRISPRi screens discovered 37 and 69 genes (FDR<0.1), respectively, that when upregulated or downregulated caused exhaustion/dysfunction resistance (Table 1).

T cell therapeutics for cancer are currently of great interest in industry. Discoveries from these screens can be used to reprogram T cells for increased efficacy, potency, durability, and scope. This is particularly the case for solid tumors, where durable successes for T cell therapies have been limited, and T cell exhaustion/dysfunction is a major hurdle. Genes discovered in the CRISPRa screen can be engineered for overexpression by cDNA transgene delivery or promotor/epigenetic engineering at the endogenous locus. Conversely, genes discovered in the CRISPRi screen can be interfered with by gene knockout or promotor/epigenetic engineering. Additionally, protein products of the discovered genes can be activated or inhibited through biologics or small molecules to enhance T cell function, either post adoptive T cell therapy, as a standalone immunotherapy, or in combination with other immunotherapies such as bispecific T cell engagers or checkpoint blockade.

Current FDA approved T cell therapies include retroviral engineered CAR T cells for B cell derived hematologic cancers, where an extracellular B cell antigen-recognizing antibody is with an intracellular CD3ζ and co-receptor signaling domains to mediate T cell activation and effector response, including proliferation, production of cytokines, and target cell killing. These CAR T cell therapies generally do not have additional genetic modifications and are limited by primary and acquired resistance. Furthermore, T cell therapies have been limited in solid tumors, due to incomplete clearance and T cell exhaustion. Reprogramming T cells to overexpress or downregulate additional key genes identified by this invention can lead to increased T cell therapeutic function and decreased resistance, increasing the efficacy, potency, durability, and scope of these products.

### Modulating T Cell Exhaustion/Dysfunction

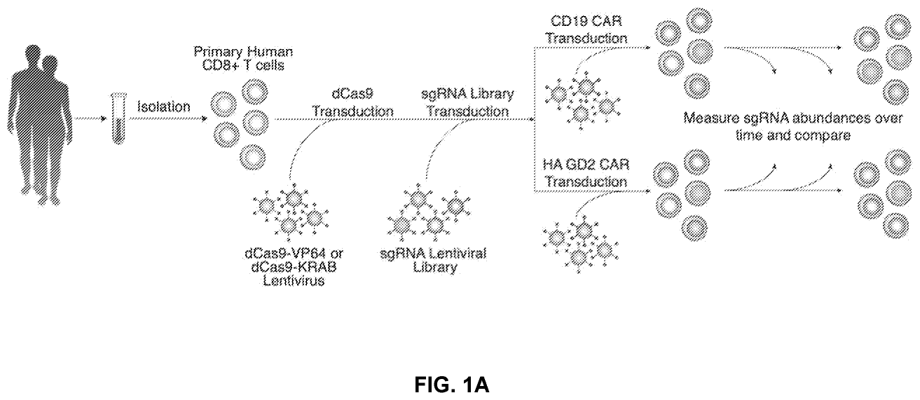

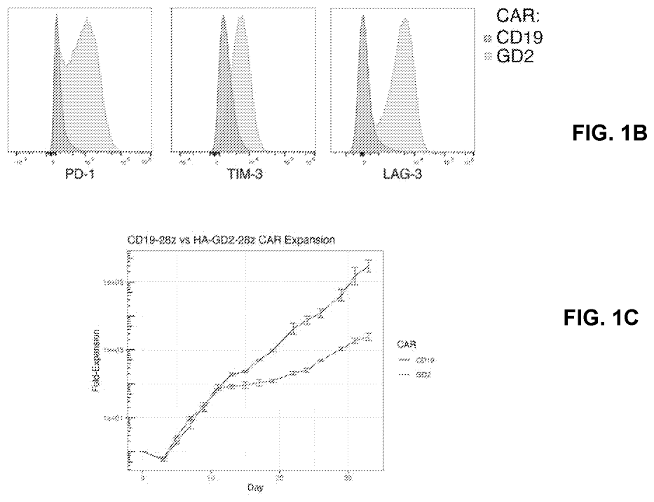

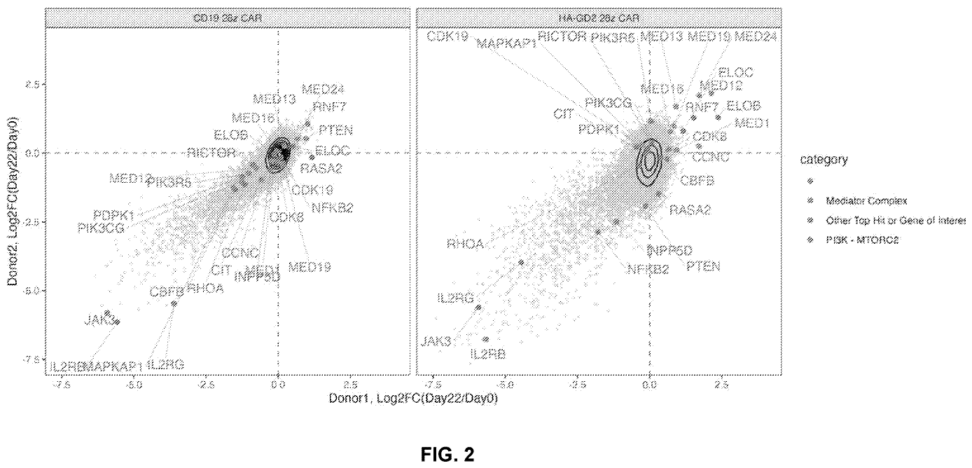

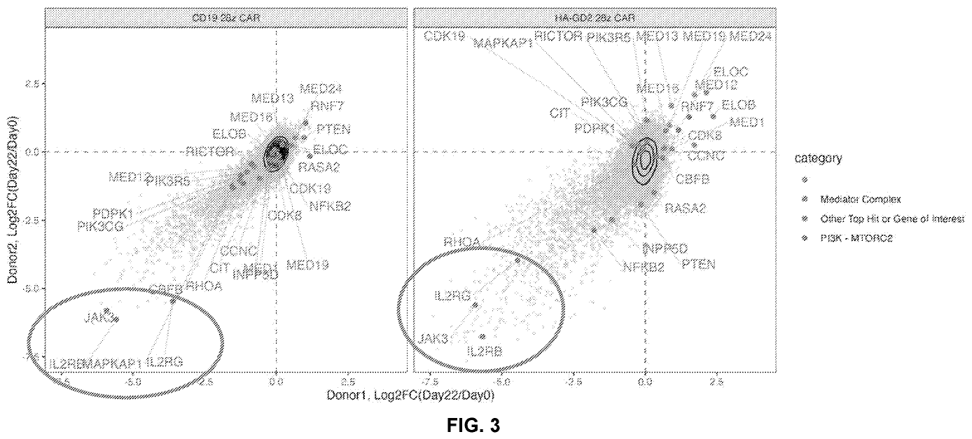

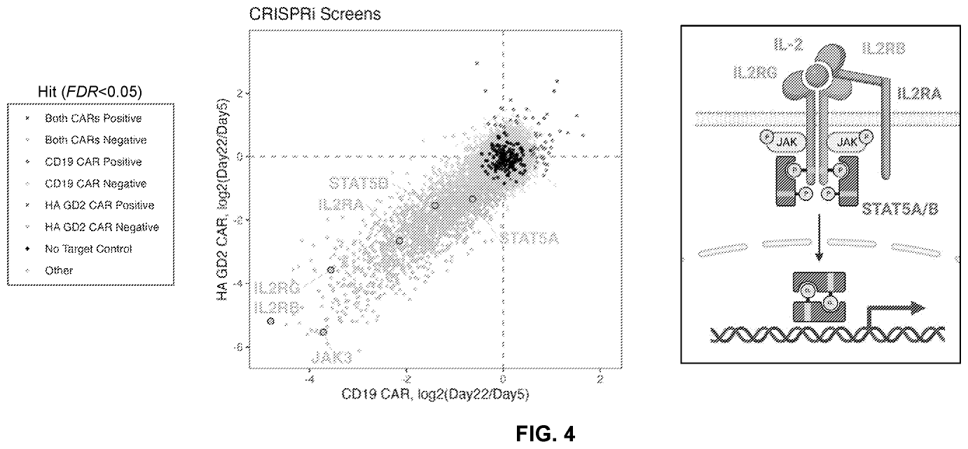

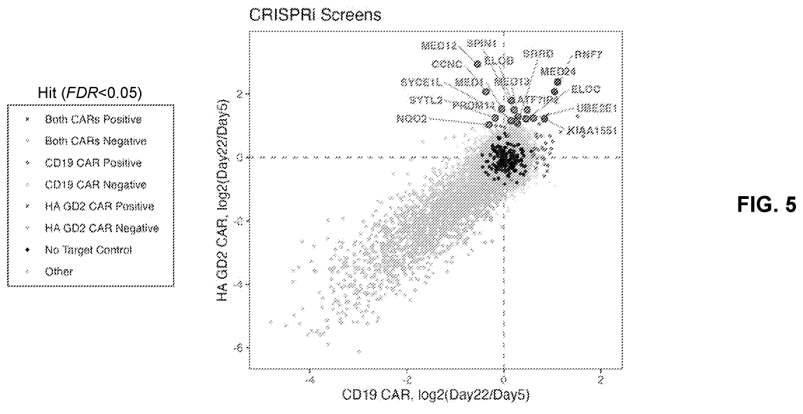

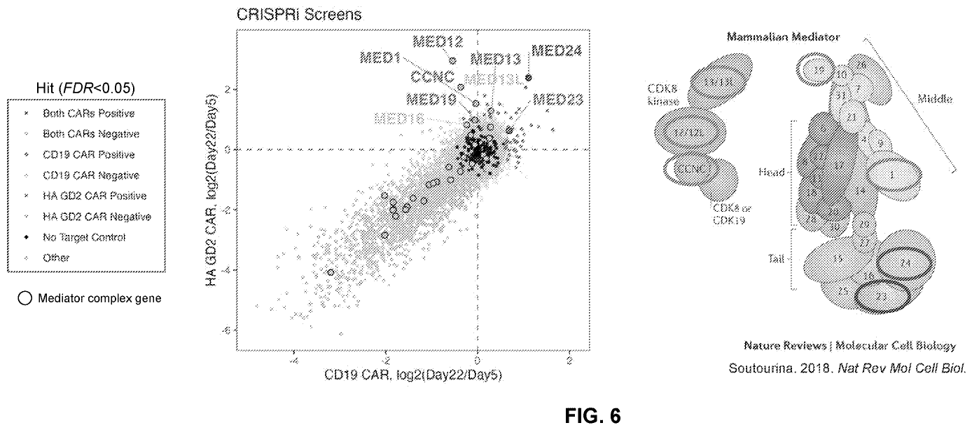

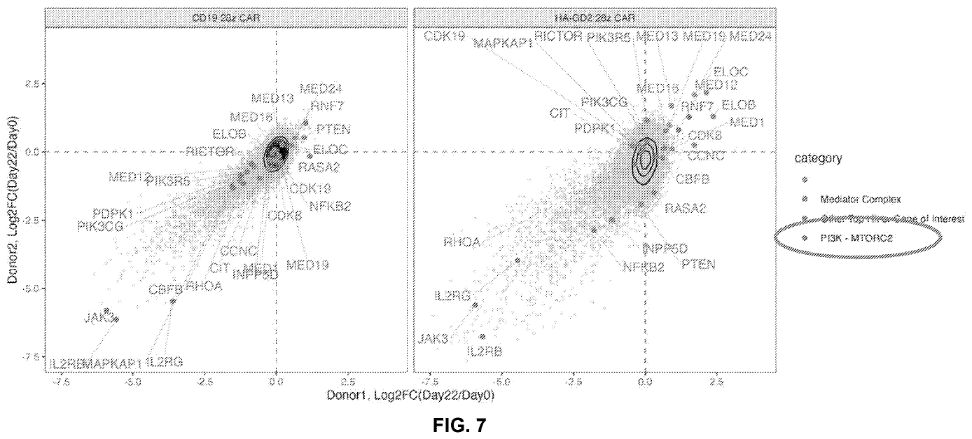

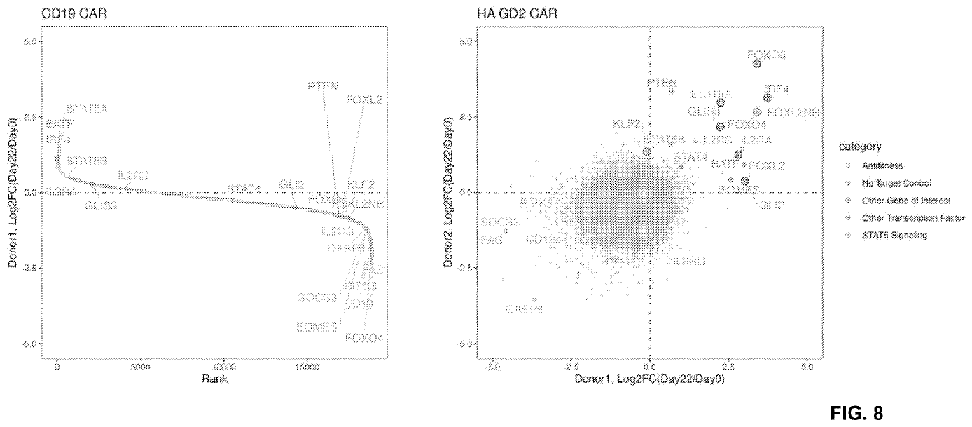

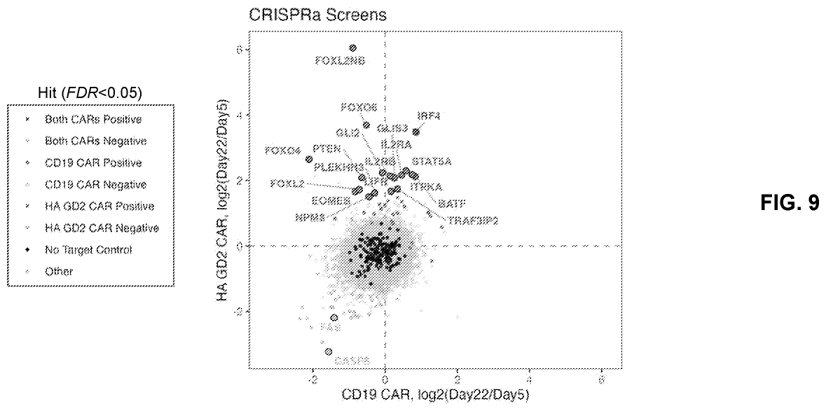

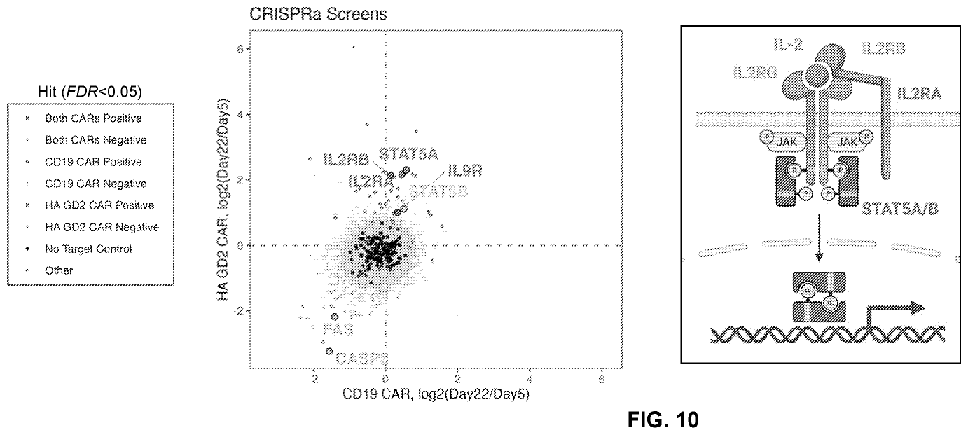

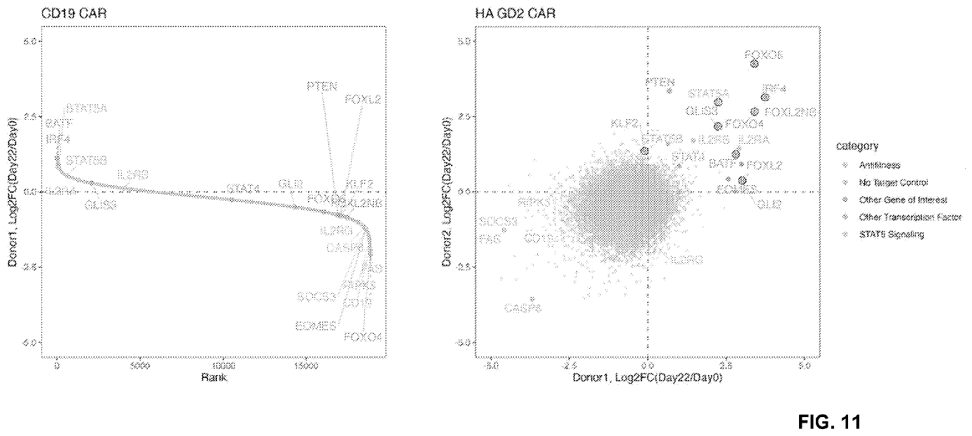

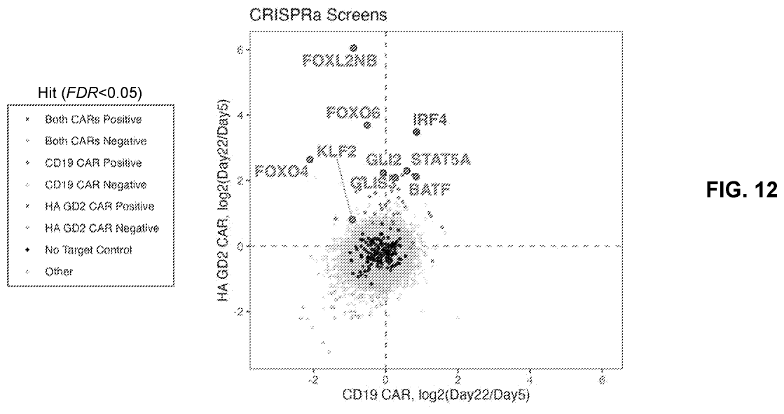

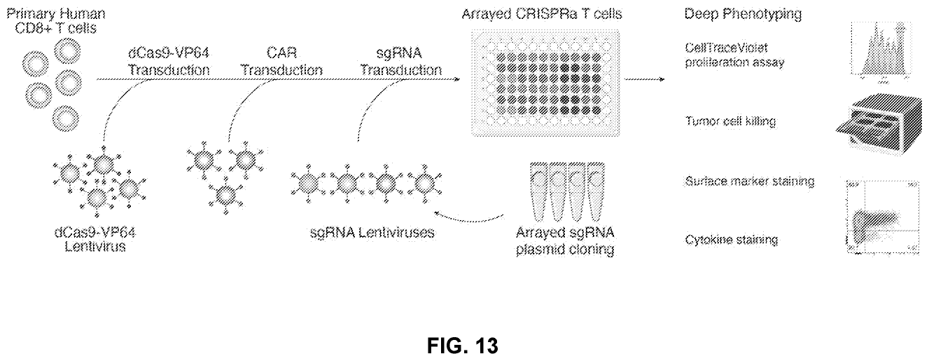

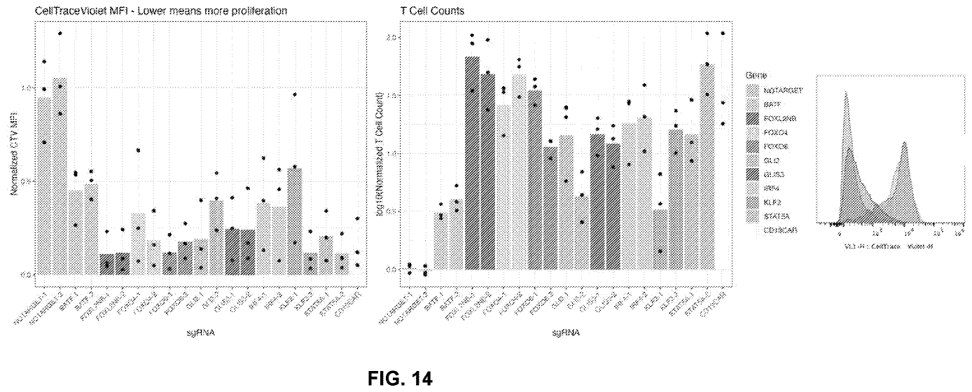

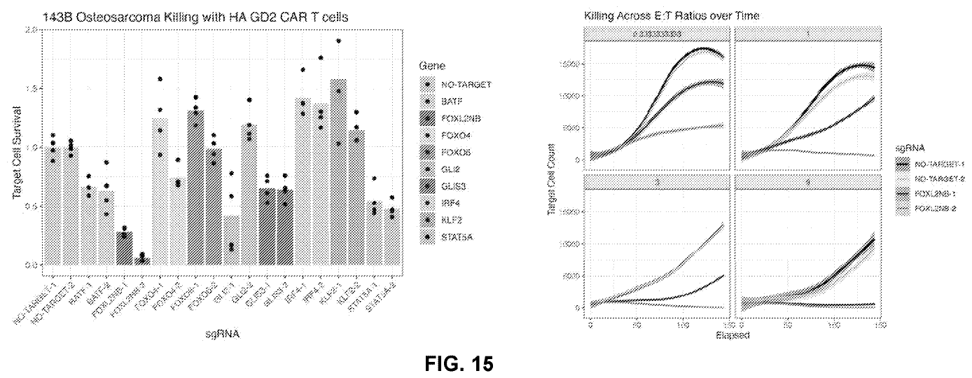

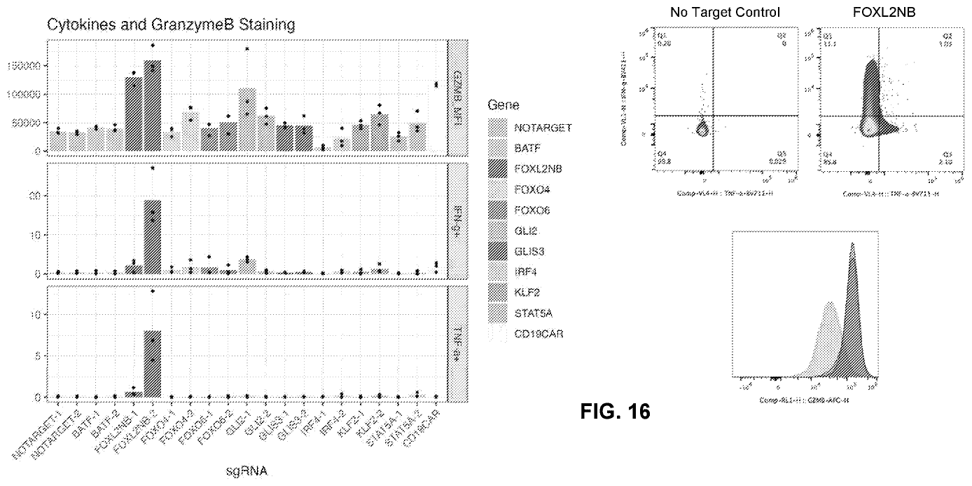

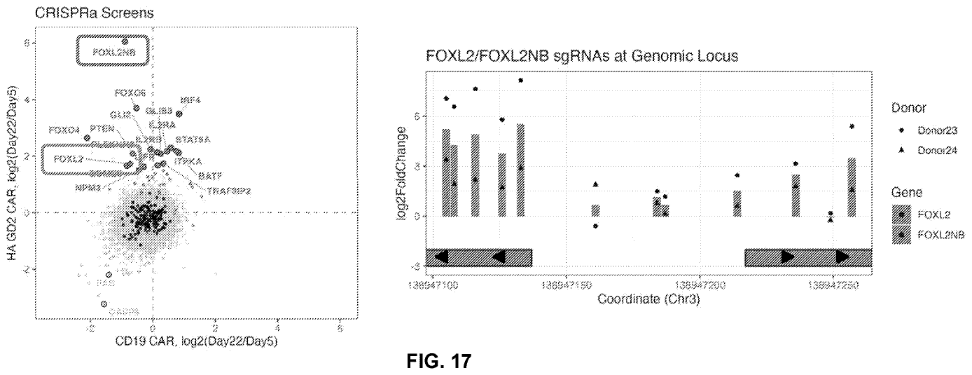

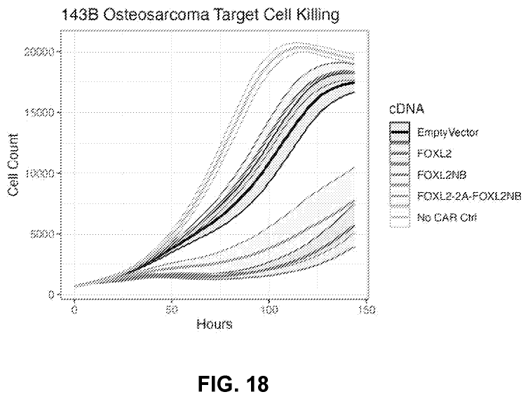

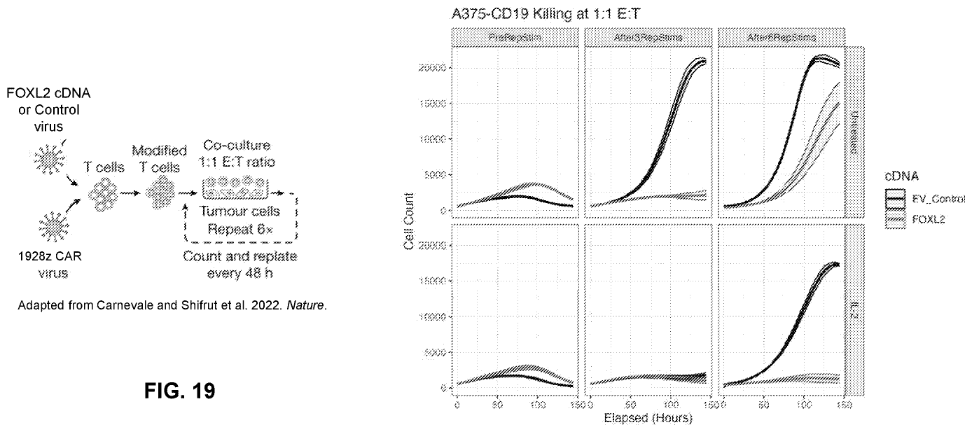

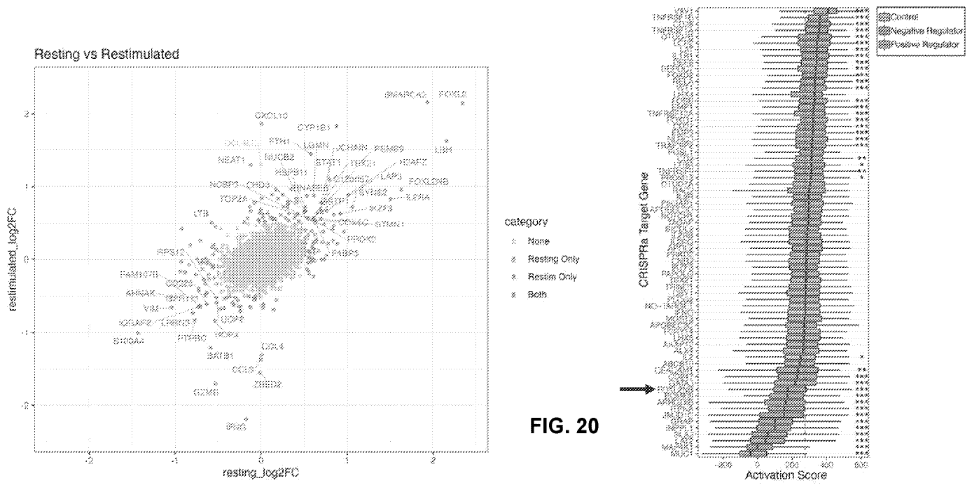

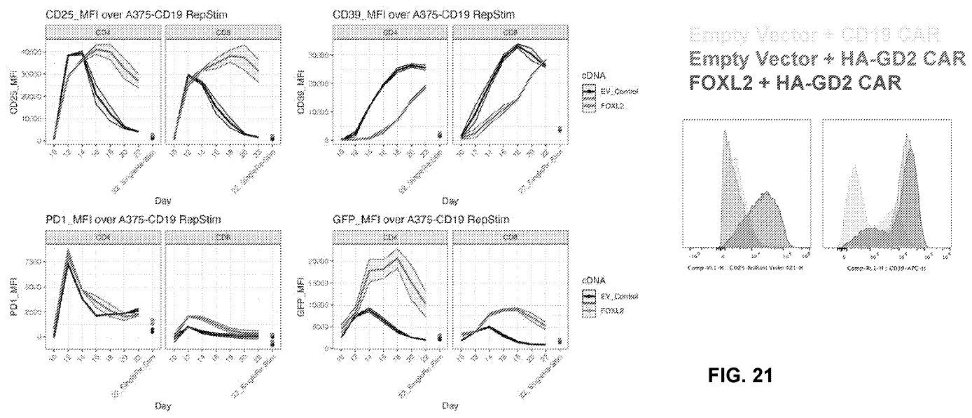

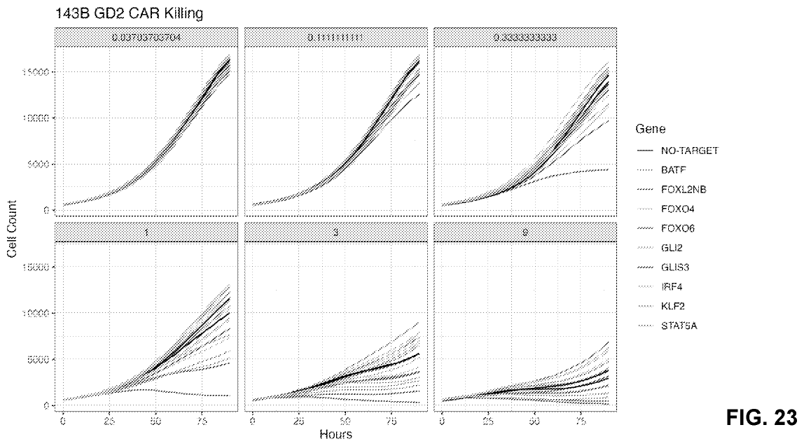

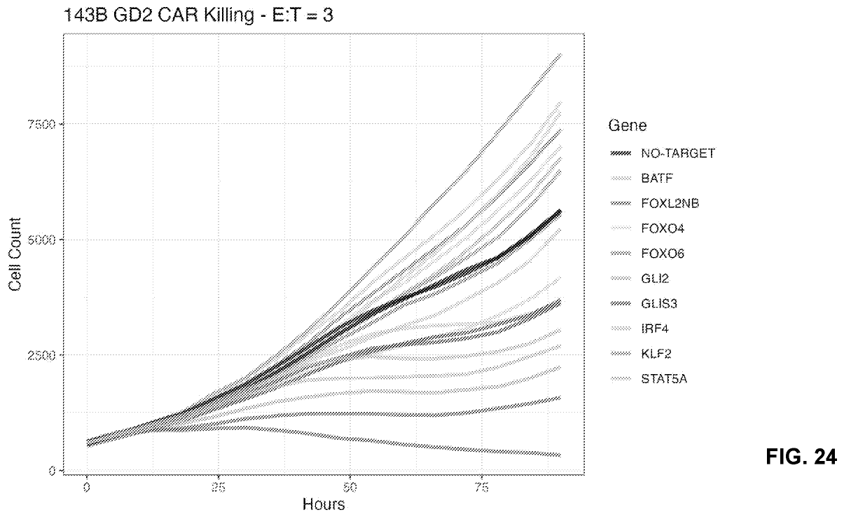

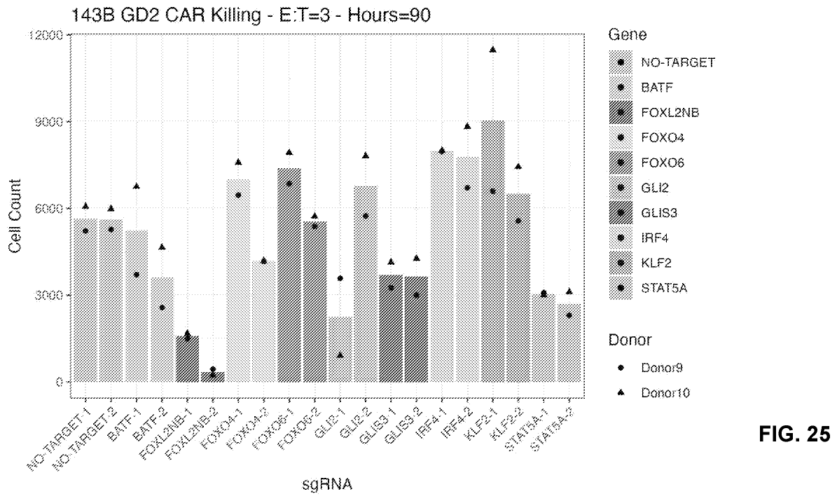

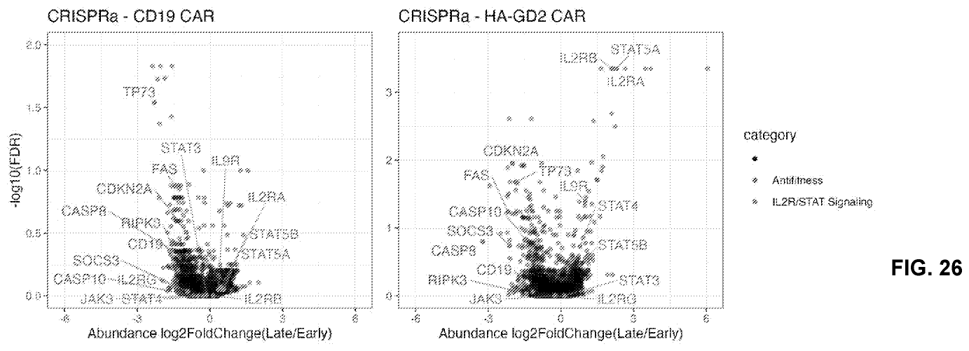

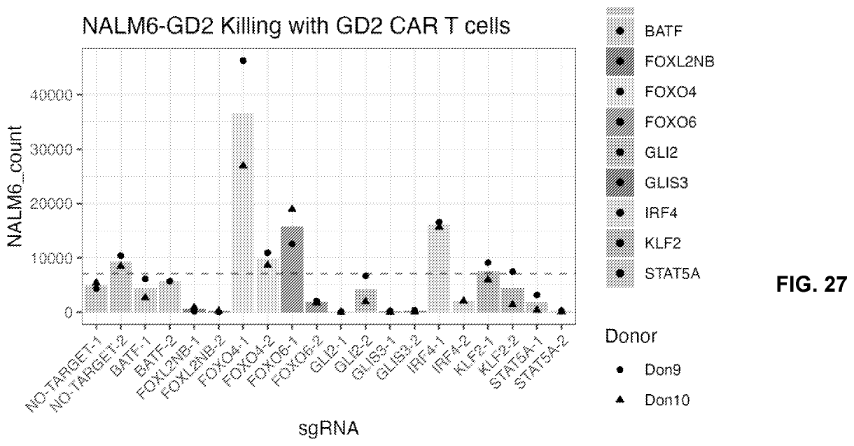

Genes that are upregulated or downregulated, respectively, to cause exhaustion/dysfunction resistance are provided in Table 1 or FIGS. 1-27. Any of the genes or the proteins they code are regulators of T cell exhaustion/dysfunction resistance that can be used in the methods and compositions described herein. Agents that modulate the listed regulators can also be used in the methods and compositions described herein. For example, to positively regulate T cell exhaustion/dysfunction one or more expression cassettes encoding one or more T cell exhaustion/dysfunction regulators, one or more agents that increase the expression or activity of such regulator, or agents that inhibit regulators of T cell exhaustion/dysfunction can be used. In one embodiment, antibodies, one or more expression cassettes encoding one or more T Cell exhaustion/dysfunction regulators, one or more agents that increase the expression or activity of such regulator, or agents that inhibit regulators of T cell exhaustion/dysfunction can be used. Agents that can modulate the T cell exhaustion/dysfunction regulators can include expression vectors, inhibitory nucleic acids, antibodies, small molecules, guide RNAs, nucleases (e.g., one or more cas nucleases), nuclease-dead cas variants (e.g., dCas9-VP64, dCas9-KRAB), or a combination thereof.

For example, T cells and other types of cells can be modified ex vivo to increase or decrease any of the T cell exhaustion/dysfunction regulators listed in Table 1 or FIGS. 1-27, and the modified cells can be administered to a subject that can benefit from such administration. In another example, the expression or activity of any of the T cell exhaustion/dysfunction regulators listed in Table 1 or FIGS. 1-27 can be modulated by in vivo administration of expression vectors, virus-like particles (VLP), CRISPR-related ribonucleoprotein (RNP) complexes, and combinations thereof that include or target any of the regulators listed in Table 1 or FIGS. 1-27. The regulator nucleic acids, regulator protein, regulator guide RNAs and CRISPR nucleases can be introduced via one or more vehicles such as by one or more expression vectors (e.g., viral vectors), virus like particles, ribonucleoproteins (RNPs), nanoparticles, liposomes, or a combination thereof. The vehicles can include components or agents that can target particular cell types (e.g., antibodies that recognize cell-surface markers), facilitate cell penetration, reduce degradation, or a combination thereof.

In addition, new agents can be identified by screening methods described herein that include, for example, evaluating assay mixtures containing one or more test agents and a population of T cells after incubation of the assay mixtures for a time and under conditions sufficient for determining whether the test agent can modulate the expression or activities of any of the regulators described herein. In some cases, the assay mixtures can include T cells and other types of cells, for example, other immune cells such as those that can interact with T cells. Useful test agents identified by such methods can, for example, increase or decrease the expression or activities of any of the regulators listed in any of Table 1 or FIGS. 1-27.

Hence, any of the regulators of T cell exhaustion/dysfunction, as well as agents that can modulate those regulators (i.e., modulators), can be used in the methods and compositions described herein.

The regulators and/or the agents that modulate the regulators can be evaluated by various assay procedures. Such assay procedures can also be used to identify new T cell exhaustion/dysfunction regulators. In some cases, the assay procedures can be used to evaluate the utility of a type (positive or negative effect), quantity, or extent of a regulator or modulating agent activity on T cell activity or T cell numbers.

For example, the methods for evaluating Applicants' regulators/agents or new regulators/agents can involve contacting one or more T cells (or a T cell population) with a test agent to provide a test assay mixture, and evaluating the test assay mixture for at least one of
Detecting and/or quantifying cytokine (e.g., interferon-γ (IFN-γ, interleukin-2(IL-2)) production;
Quantifying the numbers of T cells within the test assay mixture;
Detecting proliferation, such as via quantification of a dye that dilutes with cell divisions;
Detecting whether T cells in the test assay mixture express, e.g., an increase in expression or a decrease in expression, one or more of the regulators described herein;
Quantifying the number of cells that express, e.g., an increase in expression or a decrease in expression, one or more of the regulators expressed by a population of T cells; or
A combination thereof.

The T cells or T cell populations that are contacted with the test agent/test regulator can also include a variety of lymphoid and/or myeloid immune cells. For example, test agents can be introduced into an assay mixture that contains cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune (e.g., lymphoid and/or myeloid) cells, or a combination thereof.

Test agents that exhibit in vitro activity for resistance to exhaustion of T cells or for modulating the amount or activity of any of the regulators described herein can be evaluated in animal disease models. Such animal disease models can include cancer disease animal models, immune system disease models, or combinations thereof.

### Genes Upregulated for T Cell Resistance/Dysfunction

The following genes are upregulated in T cells resistant to exhaustion/dysfunction (see Table 1): BATF, CUL3, CLASRP, CDKL3, EOMES, FOXL2NB, FOXO6, FOXO4, FOSB, FOSL1, FOXL2, GLIS3, GLI2, IRF4, IL2RA, INPP5D, IL2RB, ITPKA, ITPKB, IL10, IL9R, LIFR, LCK, LYN, NPM3, NQO1, PLEKHH3, PTEN, RFLNB, STAT5A, SHOC2, S100A13, TRAF3IP2, TMEM167B, TSR2, VAV1, and ZNF211.

Sequences and other information relating to these genes, and their encoded proteins, is available, for example from the NCBI and UniPROT databases, which are incorporated by reference.

A few examples of human protein sequences encoded by some of the genes detected as regulators of T cell exhaustion/dysfunction resistance are provided.

For example, an amino acid sequence for the protein encoded by the human basic leucine zipper transcription factor, ATF-like gene, also known as BATF, that is a regulator of T cell exhaustion/dysfunction resistance is available from the UniPROT database at accession no. Q16520 or accession number NP_006390, cDNA NM_006399 with gene location at Chr 14: 75.52-75.55 Mb.

| (SEQ ID NO: 1) | |
|---|---|
| 1 | aaagcgagcg acatgtccct ttggggagca gtccctctgc accccagagt gaggaggacg |
| 61 | caggggtcag aggtggctac agggcaggca gaggaggcac ctgtaggggg tggtgggctg |
| 121 | gtggcccagg agaagtcagg aagggagccc agctggtgac aagagagccc agaggtgcct |
| 181 | ggggctgagt gtgagagccc ggaagatttc agccatgcct cacagctccg acagcagtga |
| 241 | ctccagcttc agccgctctc ctccccctgg caaacaggac tcatctgatg atgtgagaag |
| 301 | agttcagagg agggagaaaa atcgtattgc cgcccagaag agccgacaga ggcagacaca |
| 361 | gaaggccgac accctgcacc tggagagcga agacctggag aaacagaacg cggctctacg |
| 421 | caaggagatc aagcagctca cagaggaact gaagtacttc acgtcggtgc tgaacagcca |
| 481 | cgagcccctg tgctcggtgc tggccgccag cacgccctcg ccccccgagg tggtgtacag |
| 541 | cgcccacgca ttccaccaac ctcatgtcag ctccccgcgc ttccagccct gagcttccga |
| 601 | tgcggggaga gcagagcctc gggaggggca cacagactgt ggcagagctg cgcccatccc |
| 661 | gcagaggccc ctgtccacct ggagacccgg agacagaggc ctggacaagg agtgaacacg |
| 721 | ggaactgtca cgactggaag ggcgtgaggc ctcccagcag tgccgcagcg tttcgagggg |
| 781 | cgtgtgctgg accccaccac tgtgggttgc aggcccaatg cagaagagta ttaagaaaga |
| 841 | tgctcaagtc ccatggcaca gagcaaggcg ggcagggaac ggttattttt ctaaataaat |
| 901 | gctttaaaag aaa |

(SEQ ID NO: 2)
MPHSSDSSDSSFSRSPPPGKQDSSDDVRRVQRREKNRIAAQKSRQRQTQKADTLHLESEDLEKQNAALRKEIKQLTE
ELKYFTSVLNSHEPLCSVLAASTPSPPEVVYSAHAFHQPHVSSPRFQP

An amino acid sequence for the protein encoded by the CUL3 gene (Culling 3 protein) that is a regulator of T cell exhaustion/dysfunction resistance is available from the UniPROT database at accession no. Q13618 or accession number NP_001244126. NP_001244127 or NP_003581. cDNA NM_001257197. NM_001257198 or NM_003590 with gene location at Chr 2: 224.47-224.59 Mb.

| (SEQ ID NO: 3) | |
|---|---|
| 1 | gaggacgacg ttcggcctgc gcagtgagat gtttgtccgt cgccgccgcc gccgccatcg |
| 61 | cggaggagcg cgataaaggg agccgagccg gacgtgaggg ggaccccgcg gagccgccgc |
| 121 | gccagcgcag ccccccagcc gcatcggagt cgccagagtc cgagccgccg ccgccgccgc |
| 181 | cgccgccccc gccgccgccg cggctgccca ggggccggcc agctccccag ccctgcccgg |
| 241 | gggtcgcgcc gccgccgccc ccagcgtcgc ccctagcctg cctgcccgcc ttaaatgtga |
| 301 | caccggcgtc gcggagcgcg acctgaagcc gccgccgggg aggggacgag caccatgtcg |
| 361 | aatctgagca aaggcacggg cagccggaag gacaccaaga tgcggatccg ggcctttccg |
| 421 | atgaccatgg atgaaaaata tgtaaacagc atttgggacc ttctgaaaaa tgcaattcaa |
| 481 | gaaatccagc gtaagaataa cagtggtctt agttttgagg agctctatag aaatgcatat |
| 541 | acaatggttt tgcataaaca tggagaaaag ctctacactg gactaagaga agttgttacc |
| 601 | gaacatctca taaataaggt gcgagaagat gtactaaatt cattgaataa caactttctt |
| 661 | caaacgctaa atcaagcttg gaatgatcat caaacagcta tggtgatgat tagagacata |
| 721 | ctaatgtaca tggaccgtgt gtatgtacaa caaaataatg tggagaacgt ctacaatttg |
| 781 | ggattaatta tttttcgaga tcaagttgta cgttatgggt gtattaggga tcatctacgg |
| 841 | caaactctat tggatatgat tgcaagagag cggaaaggag aagtcgtaga cagaggcgca |
| 901 | ataagaaatg cttgccagat gttaatgatt ttaggtctcg aaggaagatc agtctatgaa |
| 961 | gaagattttg aggctccttt tttggaaatg tctgcagaat tttttcagat ggaaagccag |
| 1021 | aaatttttag cagaaaatag tgcttcagta tatataaaga aagtagaagc tagaattaat |
| 1081 | gaagaaatag aacgagtgat gcactgcctt gacaaatcaa cggaagaacc aattgtaaag |
| 1141 | gtggttgaaa gggaactcat ttccaagcac atgaagacta tagtagaaat ggagaattct |
| 1201 | gggctagtac atatgttgaa aaatggaaag acagaagacc ttggttgcat gtacaagtta |
| 1261 | tttagtcgtg tgccaaatgg tttgaaaaca atgtgtgagt gtatgagttc ctatttgagg |
| 1321 | gagcaaggta aagctcttgc ttctgaagaa ggagaaggaa agaatcctgt tgactatatc |
| 1381 | cagggcttat tggatctgaa gagtaggttc gatcgcttcc tcctggaatc attcaacaat |
| 1441 | gaccgtctct ttaaacaaac tattgcgggt gactttgagt attttctcaa cctcaactcc |
| 1501 | aggtctcctg aatacctctc attatttatt gatgataagc tgaaaaaggg agtcaaaggg |
| 1561 | ctaacagaac aagaagtaga aacaatattg gataaagcaa tggtcctttt taggtttatg |
| 1621 | caagaaaaag atgtatttga acgttattat aaacaacact tggcaaggag acttctcaca |
| 1681 | aataaaagtg tttctgatga ctctgaaaaa aacatgatat ctaagttaaa gactgaatgt |
| 1741 | ggatgtcagt tcacgtcaaa actggaagga atgtttaggg atatgagcat ctcaaacaca |
| 1801 | acgatggatg aattcaggca acatctacag gcaactggtg tatctttagg tggtgttgat |
| 1861 | cttacagtcc gggtgctcac gacaggatat tggcccactc agtcagccac accaaagtgc |
| 1921 | aacatcccac cagcaccaag acatgctttt gagatattca gaaggttcta cttagccaaa |
| 1981 | cacagtggtc gacagctcac actccagcat catatgggtt ctgcagatct caatgccaca |
| 2041 | ttttatggac cagttaaaaa ggaagatgga tctgaagttg gtgttggagg tgcacaagta |
| 2101 | actggctcta atacacggaa gcacatattg caagtttcca ctttccagat gaccatatta |
| 2161 | atgctcttta ataatagaga aaaatacaca tttgaggaaa ttcagcaaga gacagatatc |
| 2221 | cctgaaagag agcttgttag agccctacag tcccccgcct gtggtaaacc aacacagcgg |
| 2281 | gttcttacaa aagaacccaa atcaaaggaa atagaaaatg gtcatatatt tacagttaat |
| 2341 | gatcaattca catccaaact acacagagtc aagattcaaa cagttgctgc caaacaaggt |
| 2401 | gaatccgacc cagagaggaa agaaacaagg cagaaagtag acgacgacag aaaacatgag |
| 2461 | atagaagctg ctatagtgcg gataatgaaa tctagaaaga agatgcagca caatgttcta |
| 2521 | gtagcggagg taactcagca gttgaaggcg cgattcttac caagtccagt tgttattaag |
| 2581 | aaacgtattg aaggacttat tgagagagaa tattcggcac gaacacctga ggatcgcaaa |
| 2641 | gtatacacat atgtagcata aaatgcgttc agaaatttga tttattcttg gactgtactc |
| 2701 | ttcgcatgga ctgggaagtt cttttaaatc attaaatatt aagacgacca tctcttctat |
| 2761 | taaattacag tacatgttct agaccattga gatcaagcct ttactccctt tgagagtttc |
| 2821 | caacatcagt tgattgagct tcaggcttta caacgtttat ccctgtagag atcatcttta |
| 2881 | cagttcctcg ggaaaatgtg aatgtgctgc gttttgtttt ctttactgta tgaaaacagg |
| 2941 | aaaataaaag agaaatttag aaaatacagc tcattacaat aaaattgttg gatttcattt |
| 3001 | ccccaggtct tcagtgttga tgtaaatgtg ttttgtagtg ttgcttagca ctttgcgcat |
| 3061 | tgtgtaagtt gggtaacaaa aatggcaaaa gaaatgcaga tttcccagtt atcttgctct |
| 3121 | gaagacattt ctttagctag tcttatttaa ttttaaaggt ttgataaata tacaaagacc |
| 3181 | caagcataca atttgagcat gctgtattct accacttgca cttactcatc ttctagcctt |
| 3241 | atgaccagga acctttgttt tyggctcaag tacacatcct gatttggttt ctccctgttg |
| 3301 | taatttgaga tttcccaaat taagtcttat aatgattagg ttttgttctg agttatttct |
| 3361 | agagaaagaa agtattttag tatgtgtaaa ttgtacaata atttttttgc tgttgtactc |
| 3421 | actgctaata gtggattgta tagggtggtg tttttatttc atttattgta gacaaaaata |
| 3481 | atgaatttag tatacacata cccactaatt caagtatgtc agagcacaaa gttggcagct |
| 3541 | ggttatattt aattcagccc ctctgaaaag cacatacagt ctatactttg tttataaata |
| 3601 | ccctaggtcc tccctgcccc taaatatata ttttggtgaa gctgtggtgc actattaatt |
| 3661 | ttctgtttca aaatgcaatc taaagatgca ataaatatga tgaccttgat agttttaatg |
| 3721 | aaatcttcaa ttcttgcagc tgtgttttgg aaagtgatta agcaatattt ctcaaacctg |
| 3781 | atgattcttg gatatttagc tattgtcctc caaagagtca tcgtttcacg ttttcaaaca |
| 3841 | tgtgctttgt gctgaagatt cttgtcagat atcctgtgct tccagagtat ttttatcttc |
| 3901 | aattttattt aattttgttt cattttgttt tcataagaca atgtttcaga tatgtaatgg |
| 3961 | ggcaaaccac tatagatttc tgttggatag aaaaatgaaa tgttactaat aagtttaaat |
| 4021 | tgacccttcg ttgactatca gtactgttga tacatttatt agaatgtatg tctcagaaga |
| 4081 | tggtttctgt ttaaatttat ggtcaagtta caacaactga attctaatat ggattaaaaa |
| 4141 | ctccacataa atatttttcc ccatgcgtct tgggttataa ttttcctttt agacttgcaa |
| 4201 | atatgctttt attgcaattt tgtctccatc tgtactgtag aattacaagc cttgtgctct |
| 4261 | ttatctctac attttccact tctacatgat gcaattaaga tgccaatgaa aagtattgtg |
| 4321 | aacattattc tcaggacact tttaccatac ttgagtagta ctaagtcagg tgtaacatgg |
| 4381 | aaagctaagg gtttcacctt ctgattttga cataattgga aattttctat aatggtaaga |
| 4441 | tgtttatagg aaagtaatgg tagttcatgt ttgagaattt ttttattgct ttcatttccc |
| 4501 | agttaaatga tatttaacac ctgggaaaac aggacagctc agattgtcga gttagtaaac |
| 4561 | atgagcgatg taaaaacttt atttgaaatc tcctaagtga gacattaaac attttagtgc |
| 4621 | tagtttaagc cattaaaagt aaagtttaag tttacgtcat tgtctcaagt gtggctgtaa |
| 4681 | aaatcagatc tagagtttag agacatactt gttaactttg gagcacaggt ttgatattta |
| 4741 | tgcttctaaa tacattaaaa catttctgaa ttgcagttga atggttggag attgctggcg |
| 4801 | atgtgatgct gcactgaaaa aactggtcac acaaaatgtt tatatgtttg actgcataag |
| 4861 | tattatttta ggtcataaag gttataacaa aatacattta acattgcttt ctttttagca |
| 4921 | agaggtcaca gatgctattt tttctcagtt tttaatgctt taatttgaaa gaaaaatgca |
| 4981 | tcctaaccat tccatcctgt tggtgcagag aaattttggt gatttattag aagactttta |
| 5041 | aggagggtgg atacatagtt tttcaactgc aaaaatcatg tcatctcttc agtaactgtt |
| 5101 | aaagtgtaat tctctgcctt cattatcaag tccaggacag tgtttatttt gaatcgttaa |
| 5161 | cattatttaa agactgaaaa ttcattgcaa ttagggaacg cacagcctca caaaaagtgg |
| 5221 | ttgtgattgc aaggattctt tatgtcagtg aacttaaaaa gggaatgggg gaagagatgc |
| 5281 | atgcctttct aaaggtatgc tagagtcacc tgtgtagcat tgtaattatg tactatcaaa |
| 5341 | cccccaagat gctgatgata ggccttctct gtaagaatcc ttctcatagt gaagcactgt |
| 5401 | tgatgttttg ttcaccggat atgttgggta gcagaaaagg cctagaacca gttttaagat |
| 5461 | tatgcctaca tctctagaag actagtacac tggcatcatc ttaggtttct aaatattatt |
| 5521 | taattcctta atttggaaag cagttttaat gtttacagtt taagatactc attttcagta |
| 5581 | actagtgtta aagattttta tctttgcctg tctactgtaa cttaagactt tcatagaata |
| 5641 | attttttata gagtatatgg tatattgtgt caatcttgga aggaaagctg ttgcatatca |
| 5701 | attgtaggtc atgtatttta ctatatgttg catagtattt tgcattctgt aaatacgaat |
| 5761 | gttaaaacat ttcaggtcag caaaattaga agtcatttct agtcctcaaa gcagaaataa |
| 5821 | ttagaaatgt taacatttaa gtgcttatca tatgccagat actattctga atattttatg |
| 5881 | tatattcatt catttagttg tcatattggc tttatgtgac ataactgtca ttataccagt |
| 5941 | gttgtccaag gttgctagca ggaaatggca gagccaggag ttggacctag gtttaatcct |
| 6001 | atccatattc catgttacaa attgacatga ttttccatat taaaaaattt tcatttattt |
| 6061 | tgtacctcat tcacatgaat agtaatgtga caaactaaag ttaatacatg aagcattgca |
| 6121 | atatggaatt ttgcacattt ttgttagctt tacattattt gctgatacca acttttggta |
| 6181 | ggaaggaaaa cagggaaggt gattgctgct attctggaat gtgtccattg aaaagaaaat |
| 6241 | aggcgctttg tctcctacaa tcttagagaa agttutacat acttaagtgg gtaccacaga |
| 6301 | taggcatgta ccttaaattc aaatctacag ttttacaaaa tgttttcctt tgtgtgcctt |
| 6361 | tcctatgtca aattagccag aaggaagaag agacgttaag aatggcagat aaatttaaca |
| 6421 | ggtaatgata cagagattgg ccattacgcc agctctcagg agaaaaacaa ctaggatgtc |
| 6481 | ttgaaacccg tgcatttaat tttagaaatg gcaaatttgt aagcgtgtca tttaagtaat |
| 6541 | ttgatgctca gttttagggt ctaagttgac ctcgagtgag ccatgcaaaa atagctttaa |
| 6601 | aattatacat gagcagctca aagtaaacgt gtatctttta gtaacaatga aattgtcaaa |
| 6661 | tttcaagatt ttaataacag gacagttcaa ctaataaact ttattgcata caagatttat |
| 6721 | tgcaatgtgg gaaaattaaa attctcagtc tcagaa |

(SEQ ID NO: 4)
MSNLSKGTGSRKDTKMRIRAFPMTMDEKYVNSIWDLLKNAIQEI
QRKNNSGLSFEELYRNAYTMVLHKHGEKLYTGLREVVTEHLINKVREDVLNSLNNNFL
QTLNQAWNDHQTAMVMIRDILMYMDRVYVQQNNVENVYNLGLIIFRDQVVRYGCIRDH
LRQTLLDMIARERKGEVVDRGAIRNACQMLMILGLEGRSVYEEDFEAPFLEMSAEFFQ
MESQKFLAENSASVYIKKVEARINEEIERVMHCLDKSTEEPIVKVVERELISKHMKTI
VEMENSGLVHMLKNGKTEDLGCMYKLFSRVPNGLKTMCECMSSYLREQGKALVSEEGE
GKNPVDYIQGLLDLKSRFDRFLLESFNNDRLFKQTIAGDFEYFLNLNSRSPEYLSLFI
DDKLKKGVKGLTEQEVETILDKAMVLFREMQEKDVFERYYKQHLARRLLTNKSVSDDS
EKNMISKLKTECGCQFTSKLEGMFRDMSISNTTMDEFRQHLQATGVSLGGVDLTVRVL
TTGYWPTQSATPKCNIPPAPRHAFEIFRRFYLAKHSGRQLTLQHHMGSADLNATFYGP
VKKEDGSEVGVGGAQVTGSNTRKHILQVSTFQMTILMLFNNREKYTFEEIQQETDIPE
RELVRALQSLACGKPTQRVLTKEPKSKEIENGHIFTVNDQFTSKLHRVKIQTVAAKQG
ESDPERKETRQKVDDDRKHEIEAAIVRIMKSRKKMQHNVLVAEVTQQLKARFLPSPVV
IKKRIEGLIEREYLARTPEDRKVYTYVA

An amino acid sequence for the protein encoded by the CLASRP gene that is a regulator of T cell exhaustion/dysfunction resistance is available from the UniPROT database at accession no. Q8N2M8 or accession number NP_001265368, NP_008987 or NP_008987.2, cDNA NM 001278439 or NM 007056 with gene location at Chr 19: 45.04-45.07 Mb.

| (SEQ ID NO: 5) | |
|---|---|
| 1 | ctttcatttc cgcttccggt gcgggccgcg cgcgagcgca gcggtgggag gcggcgacca |
| 61 | gccggttgag gccccaggct tggcctcacc acaatgtggc acgaggctcg gaagcatgag |
| 121 | cggaagcttc gaggcatgat ggtcgactac aagaagaggg cggagcggag acgggagtat |
| 181 | tatgaaaaga tcaagaagga cccagcccag ttcctgcagg tacatggccg agcttgcaag |
| 241 | gtgcacctgg attctgcagt cgccctggcc gctgagagcc ctgttaatat gatgccctgg |
| 301 | cagggggaca ccaacaacat gattgaccga ttcgatgtcc gtgcccacct ggaccacatc |
| 361 | cccgactaca ccccccctct gctcaccacc atctccccag aacaggagtc ggacgaacgg |
| 421 | aagtgtaact acgagcgcta cagaggcctg gtgcagaacg actttgccgg catctcagag |
| 481 | gagcagtgcc tgtaccagat ctacattgat gagttgtacg gaggcctcca gagacccagc |
| 541 | gaagatgaga agaagaagct ggcagagaag aaggcttcca tcggttatac ctacgaggac |
| 601 | agcacggtgg ccgaggtaga gaaggcggca gaaaagccag aggaggagga gtcagcggcc |
| 661 | gaggaggaga gcaactcgga cgaagatgag gtcatccccg acatcgacgt ggaggtggac |
| 721 | gtggatgaat tgaaccagga gcaggtggca gatctcaaca aacaggccac gacttatggc |
| 781 | atggccgacg gtgacttcgt caggatgctc cggaaagaca aggaggaggc agaggccatc |
| 841 | aagcatgcca aggctcttga ggaggagaag gccatgtact cgggacgccg ctctcgacgc |
| 901 | cagcggagag agtttcggga gaagcggctg aggggtcgca agatcagccc acccagctat |
| 961 | gcccgccgag acagccccac ctatgacccc tataagcggt caccctcgga gtccagctca |
| 1021 | gagtcccgct cccgctcccg ctccccgacc ccgggccgcg aggagaagat cacgttcatc |
| 1081 | accagttttg ggggcagcga tgaggaggca gccgcagccg ctgctgccgc agcagcatca |
| 1141 | ggagtcacca cagggaagcc ccccgcacct ccccagcctg gcggccccgc cccgggacgt |
| 1201 | aatgccagcg cccgccgccg ctcctcctcc tcctcctcct cctcttctgc ctcgaggacc |
| 1261 | tccagctccc gctccagctc tcgctccagc tcccgctctc gccgtggtgg gggctactac |
| 1321 | cgttccggcc gccacgcccg ctcccggtcc cgctcctggt cccgctcccg ctcccgctcc |
| 1381 | cggcgctatt cccggtcccg tagccgtggc cggcggcact caggtggggg ctcccgagac |
| 1441 | ggacaccggt actcccgctc gcccgcccgg cgtggtggtt acgggccccg gcgcagaagc |
| 1501 | aggagccgct cccactcagg ggaccgctac aggcggggcg gccggggcct caggcaccac |
| 1561 | agcagtagcc gcagccgcag cagctggtcc ctcagcccgt cccgcagtcg cagcctgact |
| 1621 | cgcagccgca gccatagccc cagccccagc cagagccgca gccgcagccg cagccgcagc |
| 1681 | cagagcccct cgccatcacc cgcaagagag aagctgacca ggccggccgc gtcccctgct |
| 1741 | gtgggcgaga agctgaaaaa gaccgaacct gccgctggta aagagacagg agctgccaaa |
| 1801 | cccaagctga cgcctcagga gaagctgaaa ctgaggatgc agaaggcgct gaacaggcag |
| 1861 | ttcaaggcgg ataagaaggc ggcacaagaa aagazgatcc agcaggagca tgagcggcag |
| 1921 | gagcgggaag acgagcttcg agccatggcc cgcaagatcc gcatgaagga gcgggaacgc |
| 1981 | cgagagaagg agagagaaga gtgggaacgc cagtacagcc ggcagagccg ctcaccctcc |
| 2041 | ccccgataca gtcgagaata cagctcttct cgaaggcgct caaggtcccg atcccgaagc |
| 2101 | ccccattacc gacattaggc agaagagtgg ggggtgggga ggacaagggg gtgggtaagg |
| 2161 | ggctcaagct gtgatgctgc tggttttatc tctagtgaaa taaagtcaaa agttatttaa |
| 2221 | ttcccgtca |

(SEQ ID NO: 6)
MWHEARKHERKLRGMMVDYKKRAERRREYYEKIKKDPAQFLQVH
GRACKVHLDSAVALAAESPVNMMPWQGDTNNMIDRFDVRAHLDHIPDYTPPLLTTISP
EQESDERKCNYERYRGLVQNDFAGISEEQCLYQIYIDELYGGLQRPSEDEKKKLAEKK
ASIGYTYEDSTVAEVEKAAEKPEEEESAAEEESNSDEDEVIPDIDVEVDVDELNQEQV
ADLNKQATTYGMADGDFVRMLRKDKEEAEAIKHAKALEEEKAMYSGRRSRRQRREFRE
KRLRGRKISPPSYARRDSPTYDPYKRSPSESSSESRSRSRSPTPGREEKITFITSFGG
SDEEAAAAAAAAAASGVTTGKPPAPPQPGGPAPGRNASARRRSSSSSSSSSASRTSSS
RSSSRSSSRSRRGGGYYRSGRHARSRSRSWSRSRSRSRRYSRSRSRGRRHSGGGSRDG
HRYSRSPARRGGYGPRRRSRSRSHSGDRYRRGGRGLRHHSSSRSRSSWSLSPSRSRSL
TRSRSHSPSPSQSRSRSRSRSQSPSPSPAREKLTRPAASPAVGEKLKKTEPAAGKETG
AAKPKLTPQEKLKLRMQKALNRQFKADKKAAQEKMIQQEHERQEREDELRAMARKIRM
KERERREKEREEWERQYSRQSRSPSPRYSREYSSSRRRSRSRSRSPHYRH

An amino acid sequence for the protein encoded by the CDKL3 gene that is a regulator of T cell exhaustion/dysfunction resistance is available from the UniPROT database at accession no. Q8IVW4, cDNA NM_001113575 with gene location at Chr 5: 13.42-13.43 Mb.

```
  (SEQ ID NO: 109)
      1 agaacgcagc aactccgccg gcgtttccat ggtggcaagt ggccggttcc ttgcggggct
    61 gagcaacaaa gtagtatcag tgttcggtgg ctccactgga actacgcaga gccagaccag
  121 cgggaccaca gaatgggctg aggcggcggc ggctgtttgg ataaagtcaa cagcgggacg
  181 tggggcgtga cgccgtagta aaagcccagc ttgaaaatgg agatgtatga aacccttgga
  241 aaagtgggag agggaagtta cggaacagtc atgaaatgta aacataagaa tactgggcag
  301 atagtggcca ttaagatatt ttatgagaga ccagaacaat ctgtcaacaa aattgcgatg
  361 agagaaataa agtttctaaa gcaatttcat cacgaaaacc tggtcaatct gattgaagtt
  421 tttagacaga aaaagaaaat tcatttggta tttgaattta ttgaccacac agtattagat
  481 gagttacaac attattgtca tggactagag agtaagcgac ttagaaaata cctcttccag
  541 atccttcgag caattgacta tcttcacagt aataatatca ttcatcgaga tataaaacct
  601 gagaatattt tagtatccca gtcaggaatt actaagctct gtgattttgg ttttgcacga
  661 acactagcag ctcctgggga catttatacg gactatgtgg ccacacgctg gtatagagct
  721 cccgaattag tattaaaaga tacttcttat ggaaaacctg tggatatctg ggctttgggc
  781 tgtatgatca ttgagatggc cactggaaat ccctatcttc ctagtagttc tgatttggat
  841 ttactccata aaattgtttt gaaagtgggc aatttgtcac ctcacttgca gaatatcttt
  901 tccaagagcc ccatttttgc tggggtagtt cttcctcaag ttcaacaccc caaaaatgca
  961 agaaaaaaat atccaaagct taatggattg ttggcagata tagttcatgc ttgtttacaa
1021 attgatcctg ctgacaggat atcatctagt gatcttttgc atcatgagta ttttactaga
1081 gatggattta ttgaaaaatt catgccagaa ctgaaagcta aattactgca ggaagcaaaa
1141 gtcaattcat taataaagcc aaaagagagt tctaaagaaa atgaactcag gaaagatgaa
1201 agaaaaacag tttataccaa tacactgcta agtagttcag ttttgggaaa ggaaatagaa
1261 aaagagaaaa agcccaagga gatcaaagtc agagztatta aagtcaaagg aggaagagga
1321 gatatctcag aaccaaaaaa gaaagagtat gaaggtggac ttggtcaaca ggatgcaaat
1381 gaaaatgttc atcctatgtc tccagataca aaacttgtaa ccattgaacc accaaaccct
1441 atcaatccca gcactaactg taatggcttg aaagaaaatc cacattgcgg aggttctgtg
1501 acaatgccac ccatcaatct aactaacagt aatttgatgg ctgcaaatct cagttcaaat
1561 ctctttcacc ccagtgtgag gttaactgaa agagcaaaaa agagacgcac ttcttcacaa
1621 tctattggac aagttatgcc taatagcagg caagaggatc caggtcctat tcaaagccaa
1681 atggagaagg gtatatttaa tgagcgaaca ggtcacagtg accaaatggc aaatgagaac
1741 aaaaggaagc tgaatttttc cagatctgac aggaaagaat tccattttcc agaattgcct
1801 gtcacaatac agtcaaaaga tacaaaagga atggaagtta aacagataaa aatgctgaag
1861 agggagtcaa agaaaacaga gtcatctaag ataccaactt tacttaacgt ggatcaaaat
1921 caagaaaaac aagagggtgg agatggccat tgcgagggga agaatttgaa gagaaacagg
1981 ttttttttct ggtagtgtct tttcttttac atag=ccaaa aaatacaaga tgacaactct
2041 tcccgtttta tttatctaca atagaagtgt gatgtgagtt gttgttaaga cagccatcca
2101 tgtgcatgag catcatccag ctttttttgt tagcaaaaca tttactgttt tcttttccct
2161 tttaagactc tgttgatgtg ataatttgat ttggaattat aaagtcatct cttctctgcc
2221 ta
(SEQ ID NO: 110)
MEMYETLGKVGEGSYGTVMKCKHKNTGQIVAIKIFYERPEQSVNKIAMREIKFLKQFHHE
NLVNLIEVFRQKKKIHLVFEFIDHTVLDELQHYCHGLESKRLRKYLFQILRAIDYLHSNN
IIHRDIKPENILVSQSGITKLCDFGFARTLAAPGDIYTDYVATRWYRAPELVLKDTSYGK
PVDIWALGCMIIEMATGNPYLPSSSDLDLLHKIVLKVGNLSPHLQNIFSKSPIFAGVVLP
QVQHPKNARKKYPKLNGLLADIVHACLQIDPADRISSSDLLHHEYFTRDGFIEKFMPELK
AKLLQEAKVNSLIKPKESSKENELRKDERKTVYTNTLLSSSVLGKEIEKEKKPKEIKVRV
IKVKGGRGDISEPKKKEYEGGLGQQDANENVHPMSPDTKLVTIEPPNPINPSTNCNGLKE
NPHCGGSVTMPPINLTNSNLMAANLSSNLFHPSVRLTERAKKRRTSSQSIGQVMPNSRQE
DPGPIQSQMEKGIFNERTGHSDQMANENKRKLNFSRSDRKEFHFPELPVTIQSKDTKGME
VKQIKMLKRESKKTESSKIPTLLNVDQNQEKQEGGDGHCEGKNLKRNRFFFW
```

An amino acid sequence for the protein encoded by the EOMES gene (eomesodermin also known as T-box brain protein 2 (Tbr2) is a protein that in humans is encoded by the EOMES gene) that is a regulator of T cell exhaustion/dysfunction resistance is available from the UniPROT database at accession no. 095936 or accession number NP_001265111. NP_001265112 or NP_005433, cDNA NM_001278182, NM_001278183 or NM_005442 with gene location at Chr 3: 27.72-27.72 Mb.

```
  (SEQ ID NO: 7)
      1 agccagctgc cgtctgcgct ttgcaaagca tgcagttagg ggagcagctc stggtgagct
    61 cagtgaacct gcctggcgcg cacttctacc cgctggagag tgcgcgaggc ggcagcggcg
  121 ggagcgctgg ccacctcccc agcgcggccc cctctcctca gaagttggac ttagacaaag
  181 cgtccaagaa gttttccggc agtctctcct gcgaggcggt gagcggggag cccgcagccg
  241 ccagcgcagg ggcccccgcg gccatgctta gtgacaccga cgccggggac gcatttgcca
  301 gcgctgcggc agtggccaag ccggggcccc cggacggccg caagggctcc ccctgcgggg
  361 aggaggagct gccctccgcc gctgcagccg ccgccgccgc cgccgccgcg gctgcggcca
  421 ctgcgcgcta ctccatggac agcctgagct ccgagcggta ctacctccag tcccccggtc
  481 ctcaggggtc ggagctggct gcgccctgct cactcttccc gtaccaggcg gcggctgggg
  541 cgccccacgg acctgtgtac ccggctccta acggggcgcg ctacccctac ggctccatgc
  601 tgccccccgg cggcttcccc gcggctgtgt gcccacccgg gagggcgcag ttcggcccag
  661 gagccggtgc gggcagtggc gcgggcggta gcagcggcgg gggcggcggc ccgggcacct
  721 atcagtacag ccagggggct ccgctctacg ggccgtaccc tggagccgca gcggcgggat
  781 cttgcggagg actggggggc ctgggggttc caggttctgg cttccgtgcc cacgtctacc
  841 tgtgcaaccg gcctctgtgg ctcaaattcc accgccacca aactgagatg atcattacga
  901 aacagggcag gcgcatgttt cctttcttga gcttcaacat aaacggactc aatcccactg
  961 cccactacaa tgtgttcgta gaggtggtgc tggcggaccc caaccactgg cgcttccagg
1021 ggggcaaatg ggtgacctgt ggcaaagccg acaataacat gcagggcaac aaaatgtatg
1081 ttcacccaga gtctcctaat actggttccc actggatgag acaggagatt tcattcggga
1141 aattaaaact caccaataac aaaggcgcaa ataacaacaa cacccagatg atagtcttac
1201 aatccttaca caaataccaa ccccgactgc atattgttga agttacagag gatggcgtgg
1261 aggacttgaa tgagccctca aagacccaga ctttracctt ctcagaaacg caattcattg
1321 cagtgactgc ctaccaaaac accgatatta ctcaactaaa gattgatcat aacccctttg
1381 caaaaggctt cagagacaac tatgattcca tgtacaccgc ttcagaaaat gacaggttaa
1441 ctccatctcc cacggattcc cctagatccc atcagattgt ccctggaggt cggtacggcg
1501 ttcaatcctt cttcccggag ccctttgtca acactttacc tcaagcccgc tattataatg
1561 gcgagagaac cgtgccacag accaacggcc tcctttcacc ccaacagagc gaagaggtgg
1621 ccaaccctcc ccagcggtgg cttgtcacgc ctgtccagca acctgggacc aacaaactag
1681 acatcagttc ctatgaatct gaatatactt ctagcacatt gctcccatat ggcattaaat
1741 ccttgcccct tcagacatcc catgccctgg ggtattaccc agacccaacc tttcctgcaa
1801 tggcagggtg gggaggtcga ggttcttacc agaggaagat ggcagctgga ctaccatgga
1861 cctccagaac aagccccact gtgttctctg aagatcagct ctccaaggag aaagtgaaag
1921 aggaaattgg ctcttcttgg atagagacac ccccttccat caaatctcta gattccaatg
1981 attcaggagt atacaccagt gcttgtaagc gaaggcggct gtctcctagc aactccagta
2041 atgaaaattc accctccata aagtgtgagg acattaatgc tgaagagtat agtaaagaca
2101 cctcaaaagg catgggaggg tattatgctt tttacacaac tccctaaaga gttattttaa
2161 cctcaaaaat tagctaactt tttgcagatg gacttggtgg tgttttttgt tgtcttcttt
2221 gcctaggttg ccaaaaagat gtttgccttc caccttgatg catcctgttt tgtgcaattc
2281 tctaaaagaa ggtgccaaag ctttttgatt gctgcaggta actgaaacaa acctagcatt
2341 tttaaaaaat aagattaatg gaagacttta aggtatttta aaattcgaag ggtatccaag
2401 gttctgtatt tatttattgg ggagacacta acccttcaaa gaagcaggct gtgaacattg
2461 ggtgcccagt gctatcagat gagttaaaac ctttgattct catttctatt tgtaaattct
2521 taagcaaata gaagccgagt gttaaggtgt tttgcttctg aaagagggct gtgccttccg
2581 tttcagaagg agacattttg ctgttacatt ctgccagggg caaaagatac taggcccagg
2641 agtcaagaaa agcttttgtg aaagtgatag tttcacctga ctttgattcc ttaacccccg
2701 gcttttggaa caagccatgt ttgccctagt ccaggattgc ctcacttgag acttgctagg
2761 cctctgctgt gtgctggggt ggccagtggg actcaggaga gagcaagcta aggagtcacc
2821 aaaaaaaaaa aaaaaaaaaa gggagaattt aaaagtgtac agttgtgtgt ttagatacac
2881 tatagaataa tgtggtatat attgtacaaa tagtctacat aggtgtctgg gataatgtaa
2941 aactggtgct ttggctttgt aaagaatttg caaatcactt aacagctgca ggggcaaggg
3001 gagagtttca tcatccccat gatatttggg aatattctgt ttacttctta gatagttaag
3061 aatgtattca gctactatgt actaacttga accgtgttta aggaaaactc ctatttcatc
3121 ctcttcttgc gccatccccs ctccctaact tggtaatgtg aagaaactaa aacctgatac
3181 cacagctcct ataggcattc tagagatctt ggatctttat gtacagtctt agtcattttt
3241 aataaatgtg gttcagtaag ggaa
(SEQ ID NO: 8)
MQLGEQLLVSSVNLPGAHFYPLESARGGSGGSAGHLPSAAPSPQ
KLDLDKASKKESGSLSCEAVSGEPAAASAGAPAAMLSDTDAGDAFASAAAVAKPGPPD
GRKGSPCGEEELPSAAAAAAAAAAAAATARYSMDSLSSERYYLQSPGPQGSELAAPC
SLFPYQAAAGAPHGPVYPAPNGARYPYGSMLPPGGFPAAVCPPGRAQFGPGAGAGSGA
GGSSGGGGGPGTYQYSQGAPLYGPYPGAAAAGSCGGLGGLGVPGSGFRAHVYLCNRPL
WLKFHRHQTEMIITKQGRRMFPFLSFNINGLNPTAHYNVFVEVVLADPNHWRFQGGKW
VTCGKADNNMQGNKMYVHPESPNTGSHWMRQEISFGKLKLTNNKGANNNNTQMIVLQS
LHKYQPRLHIVEVTEDGVEDLNEPSKTQTFTFSETQFIAVTAYQNTDITQLKIDHNPF
AKGFRDNYDSMYTASENDRLTPSPTDSPRSHQIVPGGRYGVQSFFPEPFVNTLPQARY
YNGERTVPQTNGLLSPQQSEEVANPPQRWLVTPVQQPGTNKLDISSYESEYTSSTLLP
YGIKSLPLQTSHALGYYPDPTFPAMAGWGGRGSYQRKMAAGLPWTSRTSPTVESEDQL
SKEKVKEEIGSSWIETPPSIKSLDSNDSGVYTSACKRRRLSPSNSSNENSPSIKCEDI
NAEEYSKDTSKGMGGYYAFYTTP
```

For example, an amino acid sequence for the protein encoded by the human FOXL2NB (FOXL2 neighbor protein) gene that is a regulator of T cell exhaustion/dysfunction resistance is available from the UniPROT database as accession no. Q6ZUU3, shown below as SEQ ID NO: 9.

```
  (SEQ ID NO: 9)
      1 MTRTPVGSAR TRPKPRKLGP QRGKALQASS RLSESPALVK KRMPDACTLG RAGIGLPKMC
    61 LHMAVRHSKA QKTGPGILQQ RQKPPAPRAS GGPALLGKRR GCSEAGSASL EPLSSSRAAA
  121 GCLNQVPLSP FLAGPRNTRR LPAPERERIE JAATLCLEGW PLRCLASKGK LHCVY
  (SEQ ID NO: 10)
      1 gagttcatct ccaagtcact ttctgtaaac gccccgcaca gcctggaccg gcctgccccc
    61 gcccagcgag cctcaggggc ccagccgaca gccaggctca cgcgcccttg aaatctgccg
  121 gtactcgctc tgcgggctgg gctgggagat gacgaggacc ccggtggggt ctgcccgcac
  181 ccggccaaag cccaggaagc tcgggcccca gcgaggaaag gcgctccaag cctcctcgcg
  241 gctttcagaa tccccagccc tggtgaagaa gaggatgcct gatgcgtgca ccctgggaag
  301 ggctggaatc ggtctcccca agatgtgcct tcacatggct gtccggcatt cgaaggctca
  361 gaaaacaggg ccgggaatcc tgcaacagcg gcagaagccg cccgcgcctc gggcttccgg
  421 cggcccagct ctactaggga agcgtcgcgg ctgctctgag gcaggcagcg cttcgctaga
  481 accactcagc tcgtcccgcg ccgccgccgg ctgcctgaac caggttccgc tgtccccttt
  541 cctagcggga ccccgaaaca cccggcggct tcccgctcct gagcgggaga gaatagagct
  601 tgctgcaacc ctctgcttgg agggatggcc tctgcggtgc ttggctagca aagggaagct
  661 tcactgtgtc tattagtaca tccccataca ctccacgcct caacaactgt cagcactcac
  721 tccctcccgg cccctcacag ggccctttgc acccacacct cagggactcg gtgtccctcc
  781 actcaggtca cgttactcca tccacttctc tctccttctc cctctgtcaa ctccaaatcc
  841 cctctagttc tccctcccct cctacttctc tcacactcac cagctacacg tactaattca
  901 gattttgcac atgttggtgg aaaacatgtc aagccaatgt gcagacccta aggcttttca
  961 cacgctgctc actttcgcat ctcacggtgc agaaggacca atgggctcca ggtttacaag
1021 cctgactccg agaagcctgt tgattctctg atgtccttgg cctgtgattc gggtgactgg
1081 gctgccacct gggtgttttc atgatgggac tgccgcacag accacagaga agctcaggta
1141 ctgagcacgt tccagataca ctttaacatg cacaggccac tcacacaggc tttatctctg
1201 tctcgaaact ctgctgagtt tgctgctcag accaacaagg tgcagcagca gacacccaca
1261 tagcaccagg tctgaagcca gtgggtatta gtctgcctgg ttgggattag caaagtcagt
1321 tactcacata tgtgcttggg agagaatagg ggagtggaga gagagagaga gatattgagg
1381 aagaggaaag agaagcgacc tcctactctg ggaagaactc acacatgaga gctgtttcct
1441 gttgttaagt gtctcactga gctcccctct ttctccccca ggaagggctt gagaggcagt
1501 agaccagagc tctgggctcc tctttacctt gctgatgttg gggtatgagt cctccaacac
1561 cattttgtcc caaggagtat gtgccccatc gtcaatcagg cagaatgcag ggcagttgtc
1621 ggcctttttc atggtggagg ccaactggga aaaaggcaga agggtctggg tcctgggcca
1681 agtgaggccc tcttccctcc aaagacccgt gggatgctct cagaggcgga ttctagggtg
1741 gtgggagctg ctgacaagtt tcctctgata tccctcatga catctatggc ccaaagccat
1801 tttgttcagc tctgaacagc gagtgccttg ccagtaggcc tcaggcttgc tggggaacat
1861 gatgtgttct taaaagttgc cttgttgcct ttctccacac ccagactgta agcgctgatg
1921 ggcagagact ctgccctcca cttctcactc agtgctcccc accaggatgg gcttaatgcc
1981 ttttaataga attagaaaat ggttctgctg gacagaattg ggaaatgcca ctttccttat
2041 aatgaagtta taatgaagtt agaatttcca agaaagggac tgtagctgag gaaaagcggt
2101 ttgatcattg acagccagct caggatctga gagttctttg ccatttgggg ttattatagc
2161 tgcatggcca tggtgctgaa ccttaggcaa gggcaaggac acctccctag ttcccagtca
2221 tggtgaggac ctgtctgaaa cattcaaact agactttact ggaaacagag aagtctctgc
2281 attcagggca gctggcttgc aaggtaaggc ctgcagtctc cacccgcacg ctaacccatg
2341 aggggatgcc agagagagcc cttccccctt ggtcctcatt cctggctcaa ttttctccca
2401 caaagcgggc actttctaaa gatgataggc aactgccatg gaggaaggca gttttagatg
2461 cctagctggc acaaagtcca gaggaaggga gggagaaggg ctgagttttg tattactgtt
2521 ctacctttgg agattttcct catgccaaga tagggtgtgt gtgtgtgtgt gtgtgtatgt
2581 gtgtgtgtat gtgtgcacta taactttatg aaacactttt tttttttttg agacagggtc
2641 tcactctgtt gcccaagctg gagtgcagtg gtgcaatctt ggcttaccgc agtctccacc
2701 tcccaggctc aagtgatccc cccatctcag cctcagcctc ccaagtagct gggactatgg
2761 gtgtgagcca acacactcag ctaatttttt tttttttttt ttggtatttt tggtagagac
2821 agggttttac catgtgggcc caactggtct tgagctcctg agctcagggt gatttgcctg
2881 cttcaacttc ccaaagtgtt gggattacag gtgtgagcca ccatgcccag tcagtttttt
2941 attttttatt taaacagttt tgggggaaca ggtggttttt gcttacatgg ataagttctt
3001 taatggtaat ttctgagatt ttggtgcact tgtcacccga gcattgtaca cggtacccag
3061 tgtgtaatct tttatccctc atccccctcc tacgcttccc cccccgagtc cccattatat
3121 aattcttttt ttcttctttt tgagacagag tctcactctg ttgcccaggc tggagtgcag
3181 tggcatgaac ttggcttact gcagcctcct gagttcaagt gattctcctg cctgaacctc
3241 ctgtgtagct gggactacag gcatgcacca ccatgcccag ctaatttttg tattttttgt
3301 agagatgggg tttcaccatg ttggccaggc tagtcttgaa ctcctaacct caagtgatct
3361 gcctattttg gcctcccaaa gtgttgggat tacaggcgtg cgccactgcg cctggtccat
3421 tatgtcattc ttatgccttt gcatcttcat agcttagctc ccacttataa atgaaaacac
3481 aggatatttg gttttccata cttgagttac ttcacttagt ataatggtct ccagctccat
3541 ccaggttgct gtgaatgcca ttattttgtt ccttuttatg gctgagtagt attccatggt
3601 gtatatatat cacattttct ttatccactc attggttgat gggcatttag ccttgttcca
3661 tatttttgta tgcagtataa cttacagatg gtaaacaata tacagcttga tgtattctga
3721 catgtaatgc agtatgtaac caccacctgg atcaagatat ggagcatttc tggcacttca
3781 gaaggttcct tcatatcttt ttccaatcaa tattgcctca aaagggaaac catatttgga
3841 tttctatcac cataaataac ctttgcctgc cttgagcctc gtataaatgg agcatatagc
3901 atgtatgcct ttatgtctag ttttttctgt gcaacatatt tttaatattc actggtcttg
3961 ttgcatgtgt cagacattta tttcttttta ttgctgtgta atattctagt atttgcttat
4021 ccatccatat gttgagggac atttgtttcc agtttttgga tatcataaat aaagctgctg
4081 tgcacattgt tgta
```

A cDNA sequence encoding the FOXL2NB protein is available at NCBI Reference Sequence: NM_001040061.3.

An amino acid sequence for the protein encoded by the FOXO6 (Forkhead box 06) gene that is a regulator of T cell exhaustion/dysfunction resistance is available from the UniPROT database at accession no. A0A1X9RU27 or accession number NP_001278210, cDNA NM_001291281 with gene location at Chr 1: 41.36-41.38 Mb.

```
  (SEQ ID NO: 11)
MAAKLRAHQVDVDPDFAPQSRPRSCTWPLPQPDLAGDEDGALGA
GVAEGAEDCGPERRATAPAMAPAPPLGAEVGPLRKAKSSRRNAWGNLSYADLITKAIE
SAPDKRLTLSQIYDWMVRYVPYFKDKGDSNSSAGWKNSIRHNLSLHTRFIRVQNEGTG
KSSWWMLNPEGGKTGKTPRRRAVSMDNGAKELRIKGKASKKKQLQAPERSPDDSSPSA
PAPGPVPAAAKWAASPASHASDDYEAWADERGGGRPLLGEAAELEDDEALEALAPSSP
LMYPSPASALSPALGSRCPGELPRLAELGGPLGLHGGGGAGLPEGLLDGAQDAYGPRA
RAGTPAYFGGCKGGAYGGGGGFGPPAMGALRRLPMQTIQENKQASFVPAAAPFRPGAL
PALLPPPPPAPRPGPVLGAPGELALAGAAAAYPGKGAAPYAPPAPSRSALAHPISLMT
LPGEAGAAGLAPSGHAAAFGGPPGGLLLDALPGPYAAAAAGPLGAAPDRFPADLDLDM
FSGSLECDVESIILNDFMDSDEMDENFDSALPPPPPGLAGAPPPNQSWVPG
  (SEQ ID NO: 12)
      1 gaggtgcagc cggcgctgag cgcggcaggc gcgggagctg gcgctggagc cggagcggcg
    61 gcggcggcag cattcaccgg gcggcggcgg cgcgggctcc ggcaccggct cgggctccgg
  121 catggtgcaa tccggcctcg tcccgggaga gcagggcccg tgcgcagggc cggcgcccgg
  181 ggagcggcgg cagcggcggt ggcggtggtg gctgcaggcg caggcaggcg gcaggtgcta
  241 gaagcggggc tgggcgaggt gtgtgcccgc tgggctcccg ggccgccgcc ccctcactgc
  301 ccaggtcttt ccctcccttc gtgctcccca gcgcgtccag ccccctgcct ccgcgcctcg
  361 gcccatggcg ccccgcggct gagacagcgc cgccagggac ccctcccgcg ggcctccccg
  421 gggcgtgcag atcccggagc cgcccgccca ccctccgcga agcctccctc ccctcctcgc
  481 ccgagccggg cgggaccatg gctgcgaagc tgcgagcgca tcaggtggac gtggacccgg
  541 acttcgcgcc gcagagccgg ccgcgctcgt gtacctggcc cctgccgcag cctgacttgg
  601 ccggcgacga ggacggagcg ctgggcgcag gggtggccga gggcgccgag gactgcgggc
  661 cggagcgccg ggctacggcc ccggcgatgg ccccagcgcc gcccctgggc gcggaggtcg
  721 gaccgctgcg gaaagcgaag agctctcggc ggaacgcgtg ggggaacctg tcctacgccg
  781 acctcatcac caaagccatc gagagcgccc cggacaagcg gctcacgctc tcgcagatct
  841 acgactggat ggtccgttac gtgccctact tcaaggataa aggcgacagc aacagctcgg
  901 ccggctggaa gaactccatc cggcacaacc tgtcgctgca cacccgtttc atccgcgtgc
  961 agaacgaggg caccggcaag agttcgtggt ggatgctgaa ccccgagggc ggaaagacag
1021 ggaagacccc gcggcgcagg gccgtgtcca tggacaacgg ggccaagttc ctgcgcatca
1081 agggcaaggc gagcaagaag aagcagctgc aggcgcccga gcgaagcccg gacgacagct
1141 ccccgagtgc gcccgccccg gggccggtgc ctgccgcagc caagtgggcc gccagccccg
1201 cctcgcacgc cagcgacgac tacgaggctt gggccgactt ccgcggcggc gggagacccc
1261 tgctcgggga ggcggccgag ctggaggacg acgaggccct ggaggccctg gcgccatcat
1321 cgccgctcat gtacccaagc cccgccagcg cgctgtcgcc ggcgctgggc tcgcgctgtc
1381 cgggtgagct gccccgcctg gccgagctgg gaggcccgct gggcctgcac ggcggcggcg
1441 gcgcggggct gcccgagggc ctgctggacg gcgcgcagga cgcgtacggg ccgcgggccc
1501 gcgccgggac gcccgcctac ttcggcggct gcaagggcgg cgcctacggc gggggcgggg
1561 gcttcgggcc gccggcgatg ggcgctctgc gccgtctgcc catgcagacc atccaggaga
1621 acaagcaggc cagcttcgtg ccggccgcgg cgcccttccg ccctggggcg ctgcccgcgc
1681 tgctgccgcc gccgccgccc gcgcccaggc ccggcccggt gctgggtgcg ccgggggagc
1741 tggcgctggc gggcgcagcc gccgcctacc ccggcaaagg ggcggccccg tacgcgccgc
1801 ccgcgccctc gcgcagtgcc ttagcccacc ccatcagcct tatgacgctg cccggcgagg
1861 cgggcgccgc gggcctggca ccgtcgggcc acgccgccgc cttcgggggc ccgcccggcg
1921 gcctcctgct ggacgctctg ccggggccct acgctgccgc cgccgccggg ccgctgggcg
1981 ccgcgcccga ccgcttcccg gccgacctgg acctcgacat gttcagcggg agcctcgagt
2041 gcgacgtgga gtccatcatc ctcaacgact tcatggacag cgacgaaatg gacttcaact
2101 tcgattcggc cctgcctcca ccgccgccgg gcctggccgg ggccccgccc cccaaccaga
2161 gctgggtgcc gggctgaggg ccgcctcccg cctccgggcg ccccgtcccg tccccagggg
2221 gcctctgtct tcccatcctg attcccgggt ccctgccccc gactctagct ccccaggagg
2281 cggccccagc ccagctaggg acccctctcg gaggccggcc gccggggaag gggagggagg
2341 ggccggggca ccccactgct cctgcccaca ctcctgagat ccaccccctt ctcctgggca
2401 ggaagcctgg gagaggaggc tgaattccag gctggctggg agtagggagg agcggggtgg
2461 gccgcctggt gtggacggtg gtcggggaag ccaactagga gatgggccag ggagcgttta
2521 caaatcttca gtttcatttg cggaggccta gccgagaccc cgcgcccacc ccaaacacgg
2581 atctgattcc cacttgacac actttcccac tggtcttagt ctcacccacc cgaagccagc
2641 aaccctctgc ggaaaactca cacctaccta tatccatcca ccctgagcag ccctccaccc
2701 ccaaatcgcc ctccgacgac cgccaccccc acagttcagt ctccccctcc catccctgcc
2761 ggccctcgct tctcccctcc cccgtcggag tcagtccctc tcttcaaccg cccccacccc
2821 ctagtactgg tctcagcttc tccagcgggc ctcagccccg tccaccccca accccgacgc
2881 ccctttctcc gcgccagttc tggcccttct cccatattta taagtgtccg gccgggacgg
2941 gcggtgggcg cggcgtcccc ggcgcgtatc gtaggcagtg taccgtggcc gtgccgtcag
3001 agtgtgcgtg tgcgtgtgtg ccgtgtcgag gctgtgtaga gtgcattgta cagcatattt
3061 tcatgaataa aattgtttta aatatt
```

An amino acid sequence for the protein encoded by the FOXO4 (Forkhead box protein 04) gene that is a regulator of T cell exhaustion/dysfunction resistance is available from the UniPROT database at accession no. P98177 or accession number NP_001164402 or NP_005929, 60 cDNA NM 001170931 or NM 005938 with gene location at Chr X: 71.1-71.1 Mb.

```
(SEQ ID NO: 13)
MDPGNENSATEAAAIIDLDPDFEPQSRPRSCTWPLPRPEIANQP
SEPPEVEPDLGEKVHTEGRSEPILLPSRLPEPAGGPQPGILGAVTGPRKGGSRRNAWG
NQSYAELISQAIESAPEKRLTLAQIYEWMVRTVPYFKDKGDSNSSAGWKNSIRHNLSL
HSKFIKVHNEATGKSSWWMLNPEGGKSGKAPRRRAASMDSSSKLLRGRSKAPKKKPSV
LPAPPEGATPTSPVGHFAKWSGSPCSRNREEADMWTTFRPRSSSNASSVSTRLSPLRP
ESEVLAEEIPASVSSYAGGVPPTLNEGLELLDGLNLTSSHSLLSRSGLSGFSLQHPGV
TGPLHTYSSSLESPAEGPLSAGEGCFSSSQALEALLTSDTPPPPADVLMTQVDPILSQ
APTLLLLGGLPSSSKLATGVGLCPKPLEAPGPSSLVPTLSMIAPPPVMASAPIPKALG
TPVLTPPTEAASQDRMPQDLDLDMYMENLECDMDNIISDLMDEGEGLDENFEPDP
  (SEQ ID NO: 14)
      1 gcagatggct gccccggcga gtggtaaaca gagacgtcag gagggggctg ctgcttggag
    61 caaaacaaaa gggaaacccg gggggcgggg ggtggggggg gttgataggg gaatcggtgc
  121 gggaataagg gaggccgcag ccgcggggac ccggacccta gtaagagtgg cgagaaggga
  181 agaggcgggg gaagggggag tcaggggagg ggcaagtgac gcgggggagc ggggcgtggg
  241 ggagggcagc aatccgggtg gaggaaattt gggaggagtg tccggggggc agcaacttaa
  301 aagggggagg gaactgcggc taaggagacg ttcggtgatg ggagcgcaat atatgagggg
  361 atacagtgcc tcaggtttaa aagagcagga agctgagtga gaggttgcag aaaaagtgtc
  421 ttcgctcggc agaggttaca ggtggcatct cagaaagagc tttgaggcta caggctgtag
  481 tcgggaaggg gatcggagaa ctgtgtgaag ggacagctta gggactagcg tcctgggact
  541 agggggaagt tcgcgacttt ctgaagactg gcaggaatgt gcctcctggc cctcgatgct
  601 tcccccctga ggggaggcat cgtgagggac tgtggcaggc ttcactgaac gctgagccgg
  661 ggaggtccaa ctccacgtat ggatccgggg aatgagaatt cagccacaga ggctgccgcg
  721 atcatagacc tagatcccga cttcgaaccc cagagccgtc cccgctcctg cacctggccc
  781 cttccccgac cagagatcgc taaccagccg tccgagccgc ccgaggtgga gccagatctg
  841 ggggaaaagg tacacacgga ggggcgctca gagccgatcc tgttgccctc tcggctccca
  901 gagccggccg ggggccccca gcccggaatc ctgggggctg taacaggtcc tcggaaggga
  961 ggctcccgcc ggaatgcctg gggaaatcag tcatatgcag aactcatcag ccaggccatt
1021 gaaagcgccc cggagaagcg actgacactt gcccagatct acgagtggat ggtccgtact
1081 gtaccctact tcaaggacaa gggtgacagc aacagctcag caggatggaa gaactcgatc
1141 cgccacaacc tgtccctgca cagcaagttc atcaaggttc acaacgaggc caccggcaaa
1201 agctcttggt ggatgctgaa ccctgaggga ggcaagagcg gcaaagcccc ccgccgccgg
1261 gccgcctcca tggatagcag cagcaagctg ctccggggcc gcagtaaagc ccccaagaag
1321 aaaccatctg tgctgccagc tccacccgaa ggtgccactc caacgagccc tgtcggccac
1381 tttgccaagt ggtcaggcag cccttgctct cgaaaccgtg aagaagccga tatgtggacc
1441 accttccgtc cacgaagcag ttcaaatgcc agcagtgtca gcacccggct gtcccccttg
1501 aggccagagt ctgaggtgct ggcggaggaa ataccagctt cagtcagcag ttatgcaggg
1561 ggtgtccctc ccaccctcaa tgaaggtcta gagcsgttag atgggctcaa tctcacctct
1621 tcccattccc tgctatctcg gagtggtctc tctggcttct ctttgcagca tcctggggtt
1681 accggcccct tacacaccta cagcagctcc cttttcagcc cagcagaggg gcccctgtca
1741 gcaggagaag ggtgcttctc cagctcccag gctctggagg ccctgctcac ctctgatacg
1801 ccaccacccc ctgctgacgt cctcatgacc caggtagatc ccattctgtc ccaggctccg
1861 actcttctgt tgctgggggg gcttccttcc tccagtaagc tggccacggg cgtcggcctg
1921 tgtcccaagc ccctagaggc tccaggcccc agcagtctgg ttcccaccct ttctatgata
1981 gcaccacctc cagtcatggc aagtgccccc atccccaagg ctctggggac tcctgtgctc
2041 acacccccta ctgaagctgc aagccaagac agaatgcctc aggatctaga tcttgatatg
2101 tatatggaga acctggagtg tgacatggat aacatcatca gtgacctcat ggatgagggc
2161 gagggactgg acttcaacht tgagccagat ccctgagtca tgcctggaag ctttgtcccc
2221 tgcttcagat gtggagccag gcgtgttcat atctactctt tacccttgag ccctccccag
2281 gaatttggga ccctgcttta gagctagggt ggggtctggt cacacacagg tgttgaagaa
2341 attataaaga taaagctgcc ccatctgggg acgatatggg gagggagatg ggaggggaaa
2401 ggggagaggg tttttctcac tgtgccaatt agggggtaag gccccctctc aggagccatc
2461 atcggctttc cccattccta cccacttagg ctttgtagca agatgagcaa tgctgttgga
2521 aatgtgaagt caccagtggc cttacccctg cctttgggag caggattttt ttgtagagag
2581 tcttatctga gctgagccag gctagctgga gcctgggatt tctatgcagt ggccccttag
2641 gccagtgatg tgcggtgggt gggctgttta ggggatctgg aagggccaag gtctgagcac
2701 tggagtggct cgccaggcca aatcaccctt agaaggctgc agataacaga aaggcttttt
2761 ataaactttt aaagaaatat aaacacaaat atagagattt tttaaccatg gcagggtgct
2821 agtggtgggc agaatgcttt tttttctttc tgaaggcttt gtgatagtga catgatacaa
2881 acactacaga caataaatat taggagacac agggaagtgg ggagaggtgg ggagtaatag
2941 taaacacagg gaagagctcc cctacggacc aggtatagag aaaggtctat gcagaaatag
3001 gttagagttt ccctaacaaa aaagctaacc caggtcccct cattccttca acttgtgcct
3061 gggagtgtgt ggtgttaggg tgcagccaca ctcttctatg acccagcatg ggttagtgct
3121 atggtgggag agtacattga aggcctggaa ttagcttggg gccagggaag ggactgggag
3181 gggagagaag agaaggaggg aaggatttag gatggtaaag ttaggtacag agacctccct
3241 gttcaaggcc cctgacagct gtccctgccc ttcttcccct tccctgactg caggggttat
3301 gtggaagtgt gtgtggcagc aggcagcggg gaggggagga acagggaagg gggagctggg
3361 gagcttggct gagggtctgg gaaatgagca gggatggggg gggatgtgga tcaggtttac
3421 tagcacctgc cagggaggcc atctggggct ccttctccac cccagccccc aaagcagccc
3481 ttcccccagt gccctttgca tcgtcccctc ccccacccct gctgtgggtt cccatcattt
3541 cctgtgtcag cgcctggcct acccagattg tatcatgtgc tagattggag tggggaagtg
3601 tgtcaaatca ataaatgaat aaattcaata aatgcctata acca
```

An amino acid sequence for the protein encoded by the FOSB gene that is a regulator of T cell exhaustion/dysfunction resistance is available from the UniPROT database at accession no. P53539 or accession number NP_001107643 or NP 006723, cDNA NM_001114171 or NM_006732 with gene location at Chr X: 71.1-71.1 Mb.

```
  (SEQ ID NO: 15)
MFQAFPGDYDSGSRCSSSPSAESQYLSSVDSFGSPPTAAASQEC
AGLGEMPGSFVPTVTAITTSQDLQWLVQPTLISSMAQSQGQPLASQPPVVDPYDMPGT
SYSTPGMSGYSSGGASGSGGPSTSGTTSGPGPARPARARPRRPREETLTPEEEEKRRV
RRERNKLAAAKCRNRRRELTDRLQAETDQLEEEKAELESEIAELQKEKERLEFVLVAH
KPGCKIPYEEGPGPGPLAEVRDLPGSAPAKEDGFSWLLPPPPPPPLPFQTSQDAPPNL
TASLFTHSEVQVLGDPFPVVNPSYTSSFVLTCPEVSAFAGAQRTSGSDQPSDPLNSPS
LLAL
  (SEQ NO: 16)
      1 attcataaga ctcagagcta cggccacggc agggacacgc ggaaccaaga cttggaaact
    61 tgattgttgt ggttcttctt gggggttatg aaatttcatt aatctttttt tttccgggga
  121 gaaagttttt ggaaagattc ttccagatat ttcttcattt tcttttggag gaccgactta
  181 ctttttttgg tcttctttat tactcccctc cccccgtggg acccgccgga cgcgtggagg
  241 agaccgtagc tgaagctgat tctgtacagc gggacagcgc tttctgcccc tgggggagca
  301 acccctccct cgcccctggg tcctacggag cctgcacttt caagaggtac agcggcatcc
  361 tgtgggggcc tgggcaccgc aggaagactg cacagaaact ttgccattgt tggaacggga
  421 cgttgctcct tccccgagct tccccggaca gcgtactttg aggactcgct cagctcaccg
  481 gggactccca cggctcaccc cggacttgca ccttacttcc ccaacccggc catagccttg
  541 gcttcccggc gacctcagcg tggtcacagg ggcccccctg tgcccaggga aatgtttcag
  601 gctttccccg gagactacga ctccggctcc cggtgcagct cctcaccctc tgccgagtct
  661 caatatctgt cttcggtgga ctccttcggc agtccaccca ccgccgccgc ctcccaggag
  721 tgcgccggtc tcggggaaat gcccggttcc ttcgtgccca cggtcaccgc gatcacaacc
  781 agccaggacc tccagtggct tgtgcaaccc accctcatct cttccatggc ccagtcccag
  841 gggcagccac tggcctccca gcccccggtc gtcgacccct acgacatgcc gggaaccagc
  901 tactccacac caggcatgag tggctacagc agtggcggag cgagtggcag tggtgggcct
  961 tccaccagcg gaactaccag tgggcctggg cctgcccgcc cagcccgagc ccggcctagg
1021 agaccccgag aggagacgct caccccagag gaagaggaga agcgaagggt gcgccgggaa
1081 cgaaataaac tagcagcagc taaatgcagg aaccggcgga gggagctgac cgaccgactc
1141 caggcggaga cagatcagtt ggaggaagaa aaagcagagc tggagtcgga gatcgccgag
1201 ctccaaaagg agaaggaacg tctggagttt gtgc-ggtgg cccacaaacc gggctgcaag
1261 atcccctacg aagaggggcc cgggccgggc ccgctggcgg aggtgagaga tttgccgggc
1321 tcagcaccgg ctaaggaaga tggcttcagc tggctgctgc cgcccccgcc accaccgccc
1381 ctgcccttcc agaccagcca agacgcaccc cccaacctga cggcttctct ctttacacac
1441 agtgaagttc aagtcctcgg cgaccccttc cccgttgtta acccttcgta cacttcttcg
1501 tttgtcctca cctgcccgga ggtctccgcg ttcgccggcg cccaacgcac cagcggcagt
1561 gaccagcctt ccgatcccct gaactcgccc tcccccctcg ctctgtgaac tctttagaca
1621 cacaaaacaa acaaacacat gggggagaga gacttggaag aggaggagga ggaggagaag
1681 gaggagagag aggggaagag acaaagtggg tgtgtggcct ccctggctcc tccgtctgac
1741 cctctgcggc cactgcgcca ctgccatcgg acaggaggat tccttgtgtt ttgtcctgcc
1801 tcttgtttct gtgccccggc gaggccggag agctggtgac tttggggaca gggggtggga
1861 aggggatgga cacccccagc tgactgttgg ctctctgacg tcaacccaag ctctggggat
1921 gggtggggag gggggcgggt gacgcccacc ttcgggcagt cctgtgtgag gattaaggga
1981 cgggggtggg aggtaggctg tggggtgggc tggagtcctc tccagagagg ctcaacaagg
2041 aaaaatgcca ctccctaccc aatgtctccc acacccaccc tttttttggg gtgcctaggt
2101 tggtttcccc tgcactcccg accttagctt attgatccca catttccatg gtgtgagatc
2161 ctctttactc tgggcagaag tgagcccccc ccttaaaggg aattcgatgc ccccctagaa
2221 taatctcatc cccccacccg acttcttttg aaatgtgaac gtccttcctt gactgtctag
2281 ccactccctc ccagaaaaac tggctctgat tggaatttct ggcctcctaa ggctccccac
2341 cccgaaatca gcccccagcc ttgtttctga tgacagtgtt atcccaagac cctgccccct
2401 gccagccgac cctcctggcc ttcctcgttg ggccgctctg atttcaggca gcaggggctg
2461 ctgtgatgcc gtcctgctgg agtgatttat actgtgaaat gagttggcca gattgtgggg
2521 tgcagctggg tggggcagca cacctctggg gggataatgt ccccactccc gaaagccttt
2581 cctcggtctc ccttccgtcc atcccccttc ttcctcccct caacagtgag ttagactcaa
2641 gggggtgaca gaaccgagaa gggggtgaca gtcccccatc cacgtggcct ctctctctct
2701 cctcaggacc ctcagccctg gcctttttct ttaaggtccc ccgaccaatc cccagcctag
2761 gacgccaact tctcccaccc cttggcccct cacatcctct ccaggaaggg agtgaggggc
2821 tgtgacattt ttccggagaa gatttcagag ctgaggcttt ggtaccccca aacccccaat
2881 atttttggac tggcagactc aaggggctgg aatctcatga ttccatgccc gagtccgccc
2941 atccctgacc atggttttgg ctctcccacc ccgccgttcc ctgcgcttca tctcatgagg
3001 atttctttat gaggcaaatt tatatttttt aatatcgggg ggtggaccac gccgccctcc
3061 atccgtgctg catgaaaaac attccacgtg ccccttgtcg cgcgtctccc atcctgatcc
3121 cagacccatt ccttagctat ttatcccttt cctggtttcc gaaaggcaat tatatctatt
3181 atgtataagt aaatatatta tatatggatg tgtgtgtgtg cgtgcgcgtg agtgtgtgag
3241 cgcttctgca gcctcggccc aggtcacgtt ggccctcaaa gcgagccgtt gaattggaaa
3301 ctgcttctag aaactctggc tcagcctgtc tcgggctgac ccttttctga tcgtctcggc
3361 ccctctgatt gttcccgatg gtctctctcc ctctgtcttt tctcctccgc ctgtgtccat
3421 ctgaccgttt tcacttgtct cctttctgac tgtccctgcc aatgctccag ctgtcgtctg
3481 actctgggtt cgttggggac atgagatttt attttttgtg agtgagactg agggatcgta
3541 gatttttaca atctgtatcs ttgacaattc tgggcgcgag tgtgagagtg tgagcagggc
3601 ttgctcctgc caaccacaat tcaatgaatc cccgaccccc ctaccccatg ctgtacttgt
3661 ggttctcttt ttgtattttg catctgaccc cggggggctg ggacagattg gcaatgggcc
3721 gtcccctctc cccttggttc tgcactgttg ccaataaaaa gctcttaaaa acgca
```

An amino acid sequence for the protein (Fos-related antigen 1 (FRA1)) encoded by the FOSL1 gene that is a regulator of T cell exhaustion/dysfunction resistance is available from the UniPROT database at accession no. P15407 or accession number NP_001287773, NP_001287784, NP_001287785, NP_001287786, or NP_005429, cDNA NM_001300844, NM_001300855, NM_001300856, NM_001300857, or NM_005438 with gene location at Chr 11: 65.89-65.9 Mb.

```
  (SEQ ID NO: 17)
MFRDFGEPGPSSGNGGGYGGPAQPPAAAQAAQQKFHLVPSINTM
SGSQELQWMVQPHFLGPSSYPRPLTYPQYSPPQPRPGVIRALGPPPGVRRRPCEQISP
EEEERRRVRRERNKLAAAKCRNRRKELTDFLQAETDKLEDEKSGLQREIEELQKQKER
LELVLEAHRPICKIPEGAKEGDTGSTSGTSSPPAPCRPVPCISLSPGPVLEPEALHTP
TLMTTPSLTPFTPSLVFTYPSTPEPCASAHRKSSSSSGDPSSDPLGSPTLLAL
  (SEQ ID NO: 18)
      1 gagtcagaac ccagcagccg tgtaccccgc agagccgcca gccccgggca tgttccgaga
    61 cttcggggaa cccggcccga gctccgggaa cggcgggcgg tacggcggcc ccgcgcagcc
  121 cccggccgca gcgcaggcag cccagcagaa gttccacctg gtgccaagca tcaacaccat
  181 gagtggcagt caggagctgc agtggatggt acagcctcat ttcctggggc ccagcagtta
  241 ccccaggcct ctgacctacc ctcagtacag ccccccacaa ccccggccag gagtcatccg
  301 ggccctgggg ccgcctccag gggtacgtcg aaggccttgt gaacagatca gcccggagga
  361 agaggagcgc cgccgagtaa ggcgcgagcg gaacaagctg gctgcggcca agtgcaggaa
  421 ccggaggaag gaactgaccg acttcctgca ggcggagact gacaaactgg aagatgagaa
  481 atctgggctg cagcgagaga ttgaggagct gcagaagcag aaggagcgcc tagagctggt
  541 gctggaagcc caccgaccca tctgcaaaat cccggaagga gccaaggagg gggacacagg
  601 cagtaccagt ggcaccagca gcccaccagc cccctgccgc cctgtacctt gtatctccct
  661 ttccccaggg cctgtgcttg aacctgaggc actgcacacc cccacactca tgaccacacc
  721 ctccctaact cctttcaccc ccagcctggt cttcacctac cccagcactc ctgagccttg
  781 tgcctcagct catcgcaaga gtagcagcag cagcggagac ccatcctctg acccccttgg
  841 ctctccaacc ctcctcgct: tgtgaggcgc ctgagcccta ctccctgcag atgccaccct
  901 agccaatgtc tcctcccctt cccccaccgg tccagctggc ctggacagta tcccacatcc
  961 aactccagca acttcttctc catccctcta atgagactga ccatattgtg cttcacagta
1021 gagccagctt ggggccacca aagctgccca ctgtttctct tgagctggcc tctctagcac
1081 aatttgcact aaatcagaga caaaatattt cccatttgtg ccagaggaat cctggcagcc
1141 cagagacttt gtagatcctt agaggtcctc tggagcccta accccttcca gatcactgcc
1201 acactctcca tcaccctctt cctgtgatcc acccaaccct atctcctgac agaaggtgcc
1261 actttaccca cctagaacac taactcacca gccccactgc cagcagcagc aggtgattgg
1321 accaggccat tctgccgccc cctcctgaac cgcacagctc aggaggcgcc cttggcttct
1381 gtgatgagct gatctgcgga tctcagcttt gagaagcctt cagctccagg gaatccaagc
1441 ctccacagcg agggcagctg ctatttattt tcctaaagag agtattttta tacaaaccta
1501 ccaaaatgga ataaaaggct tgaagctgtg gcctgagtgc ctcactggac ccagaggcca
1561 atgggagagt atttggagcc ctaggtccca gccttagctc tacagactca ctgcatgacc
1621 ttggacaaat tctttgatat ttttggactt tgtcttatct gacaagtggg gctacatccg
1681 ctcggcctca tctccgggac tg
```

An amino acid sequence for the protein (Forkhead box protein L2) encoded by the FOXL2 gene that is a regulator of T cell exhaustion/dysfunction resistance is available from the UniPROT database at accession no. P58012 or accession number NP_075555, cDNA NM_023067 with gene location at Chr 3: 138.94-138.95 Mb.

```
  (SEQ ID NO: 19)
MMASYPEPEDAAGALLAPETGRTVKEPEGPPPSPGKGGGGGGGT
APEKPDPAQKPPYSYVALIAMAIRESAEKRLTLSGIYQYIIAKFPFYEKNKKGWQNSI
RHNLSLNECFIKVPREGGGERKGNYWTLDPACEDMFEKGNYRRRRRMKRPFRPPPAHF
QPGKGLFGAGGAAGGCGVAGAGADGYGYLAPPKYLQSGFINNSWPLPQPPSPMPYASC
QMAAAAAAAAAAAAAAGPGSPGAAAVVKGLAGPAASYGPYTRVQSMALPPGVVNSYNG
LGGPPAAPPPPPHPHPHPHAHHLHAAAAPPPAPPHHGAAAPPPGQLSPASPATAAPPA
PAPTSAPGLQFACARQPELAMMHCSYWDHDSKTGALHSRLDL
  (SEQ ID NO: 20)
      1 agaacagagc gaggctcctg gcgcactagg gactccagga ggcagctccg ccagagacgc
    61 gggtcgtgct tcgggaaacc ggggggcggg gggaggggaa gagcgcagaa aagaaaaccc
  121 accaaggcgg ggactggcct gagcggggag gggcggcgag gccggagccc ctctctgttg
  181 ggcggactcc ccatggccag aggctgagct ccactcccgc cggccgctcc ctaggggaag
  241 gggaaggaga ggggagagca gcgacaggcc tccagcaagc aagcgcgggc ggcatccgca
  301 gtctccagaa gtttgagact tggccgtaag cggactcgtg cgccccaact ctttgccgcg
  361 ccagcgcctg gagcggagag cagaggcggc ccggccgcgg cgcgccggct ttgtcatgat
  421 ggccagctac cccgagcccg aggacgcggc gggggccctg ctggccccag agaccggtcg
  481 cacagtcaag gagccagaag ggccgccgcc gagcccaggc aagggcggtg ggggtggcgg
  541 cgggacagcc ccggagaagc cggacccggc gcagaagccc ccgtactcgt acgtggcgct
  601 catcgccatg gcgatccgcg agagcgcgga gaagaggctc acgctgtccg gcatctacca
  661 gtacatcatc gcgaagttcc cgttctacga gaagaataag aagggctggc aaaatagcat
  721 ccgccacaac ctcagcctca acgagtgctt catcaaggtg ccgcgcgagg gcggcggcga
  781 gcgcaagggc aactactgga cgctggaccc ggcctgcgaa gacatgttcg agaagggcaa
  841 ctaccggcgc cgccgccgca tgaagaggcc cttccggccg ccgcccgcgc acttccagcc
  901 cggcaagggg ctcttcgggg ccggaggcgc cgcaggcggg tgcggcgtgg cgggcgccgg
  961 ggccgacggc tacggctacc tggcgccccc caagtacctg cagtctggct tcctcaacaa
1021 ctcgtggccg ctaccgcagc ctccctcacc catgccctat gcctcctgcc agatggcggc
1081 agccgcagcg gctgcagcag ctgcggctgc agccgcgggc cccggtagcc ctggcgcggc
1141 cgctgtggtc aaggggctgg cgggcccggc cgcctcgtac gggccgtaca cacgcgtgca
1201 gagcatggcg ctgccccccg gcgtagtgaa ctcgtacaat ggcctgggag gcccgccggc
1261 cgcacccccg cctccgccgc acccccaccc gcatccgcac gcacaccatc tgcacgcggc
1321 cgccgcaccg ccgcctgccc caccgcacca cggggccgcc gcgccgccgc cgggccagct
1381 cagccctgcc agcccagcca ccgccgcgcc cccggcgccc gcgcccacca gtgcgccggg
1441 cctgcagttc gcttgtgccc ggcagcccga gctcgccatg atgcattgct cttactggga
1501 ccacgacagc aagaccggcg cgctgcattc gcgcctcgat ctctgagagc ccaccgcatg
1561 ccggtgcaga cggatgcgag gatgcaggga cgcgcgacgc cggccccggt cgcagccgac
1621 gacgccgccg ccagcctgac ctcacaccct ctgggcccgc ctctggagcc agcgcccagg
1681 gtccctctgt gctttttcgc tttcctaagc tcctgtcgct cctctttgtc ccctcagttt
1741 atgtcctcct gtgctcacct ccctgacctc tgtgaccttg cactcccctg gcctgaagct
1801 gcctctctgc gcgctttcta ctgggctcgt ctctttccgg agccccagcg tctcctgccc
1861 aaattcaccg cggaaagggc ccggggcgga ggtgcgaccg ggcgtcggca gcgcagacct
1921 cttggccttc tctcacaggt cggtgcgctc gctctccgcg ttccccgccc gactgccgtg
1981 cagtccatgg ctagacgcgc cggacaggac tgatggcggg accgcgctgc ccgagaaagg
2041 gacggaccaa tacgtgtgtt tgctccgcga accctcttga agctgttcag aagccgcttg
2101 ccgcggggcc cactaggcgg ggcgggggtt gggacccagc gggagccggg gcagcctggc
2161 tccacggcct gtactcggtt tacaccgcgg gcgggggggg agggaggctg cgtttcctcc
2221 gctatcagtc ccgtcgcttc gggcacctcc gggccccggc ggctggctaa tgttttgttt
2281 gaaagatcgg tcgaactttc taagagagta tttaaaaaaa aaaaaaggaa aaaaaattca
2341 ccgggcaacc ggggaagtat tgtggccttg gagtttgcta aatccaaata tgaaaatcaa
2401 aagctttagt attcctcatc ttctcttctg gaagatttgc gttagagttt ttgttgggcc
2461 ttcaaaaagc tgtgttcaga gttaggagaa tatatccaat aaaagatggt ttcgtctacc
2521 aattggggaa gtttcaccct ctccctatct gaagaaaaaa atcaaaaaca aatgtccccg
2581 gatctttcga tgcaagtcct ggaggcaggg agatcactgc ctgcctggcc cacgctgctg
2641 ggacggctcg tcctccctgc tttttgtttt tcaaacctcc tgcttctccc accttgggaa
2701 ggagaaatgt gaaacccggc agcggccgac ctaggcggtc ttgtggcccg gagccggccc
2761 ggcccgaaaa ccatagacct gcttgtactg tagcttgttg tttgggggac caaattttct
2821 agagagaact agagcactus tcttgtgttt tttttttt tttttttttt ttgccttgtc
2881 gattcccgaa taaattttgt gttccttctt ttaa
```

An amino acid sequence for the protein (GLIS family zinc finger 3) encoded by the GLIS3 gene that is a regulator of T cell exhaustion/dysfunction resistance is available from the UniPROT database at accession no. Q8NEA6, cDNA BC033899 with gene location at Chr 9: NC_000009.12 (3824127 . . . 4490465, complement).

| | |
|---|---|
| (SEQ ID NO: 21) | |
| MMVQRLGLISPPASQVSTACNQISPSLQRAMNAANLNIPPSDTRSLISRESLASTTLSLTESQSASSMKQEW | |
| SQGYRALPSLSNHGSQNGLDLGDLLSLPPGTSMSSNSVSNSLPSYLEGTESSHSPYPSPRHSSTRSHSARSK | |
| KRALSLSPLSDGIGIDENTIIRTSPTSLVAYINGSRASPANLSPQPEVYGHFLGVRGSCIPQPRPVPGSQKG | |
| VLVAPGGLALPAYGEDGALEHERMQQLEHGGLQPGLVNHMVVQHGLPGPDSQSAGLFKTERLEEFPGSTVDL | |
| PPAPPLPPLPPPPGPPPPYHAHA | LHHPELGPHAQQLALPQATLDDDGEMDGIGGKHCCRWIDCSALYDQQE |
| ELVRHIEKVHIDQRKGEDFTCFWAGCPRRYKPENARYKLLIHMRVHSGEKPNKCTFEGCEKAFSRLENLKIH | |
| LRSHTGEKPYLCQHPGCQKAFSNSSDRAKHQRTHLDTKPYACQIPGCTKRYTDPSSLRKHVKAHSSKEQQAR | |
| KKLRSSTELHPDLLTDCLTVQSLQPATSPRDAAAEGTVGRSPGPGPDLYSAPIFSSNYSSRSGTAAGAVPPP | |
| HPVSHPSPGHNVQGSPHNPSSQLPPLTAVDAGAERFAPSAPSPHHISPRRVPAPSSILQRTQPPYTQQPSGS | |
| HLKSYQPETNSSFQPNGTHVHGFYGQLQKFCPPHYPDSQRIVPPVSSCSVVPSFEDCLVPTSMGQASFDVFH | |
| RAFSTHSGITVYDLPSSSSSLFGESLRSGAEDATFLQISTVDRCPSQLSSVYTEG | |
| (SEQ ID NO: 22) | |
| 1 atcctttatg gttgaagggt gttggaggaa gcagagtttt catcagctgc tgaagtggca | |
| 61 actaatagag gtttgaatgg ccgtagaaaa atattgtgtt acttgtttgt tgtctagggg | |
| 121 ctcttggctt tgggcctcag tgcaagtcca ttggaaaagg cagctgcaac aatctagtgg | |
| 181 tcaccagcag tcccatgatg gttcagcgac tgggactcat ttcacctcca gcaagccagg | |
| 241 tctctacagc atgcaaccag atcagtccta gcttacagag ggcaatgaat gcagccaacc | |
| 301 tgaatatacc tccttcagat accaggtccc ttatttcgcg tgagtctttg gcgtccacga | |
| 361 ccttgagtct gacggaaagt cagtcggcct caagcatgaa gcaggagtgg tcccagggct | |
| 421 acagggccct cccttcgctc tccaaccacg gctczcagaa tggccttgat ctaggggatc | |
| 481 tccttagcct tcctcccggg acatccatgt ccagcaatag tgtctctaac tcattaccat | |
| 541 cctacctttt tggcacggaa agtagccact ctccttaccc tagtcctcgg cactcatcca | |
| 601 ccaggtccca ctcggcccgc tccaagaaga gagcgctgtc cttgtccccg ctgtccgatg | |
| 661 gcatcgggat agatttcaat accatcatcc gcacgtcgcc cacgtccttg gtggcctaca | |
| 721 tcaacgggtc gagggcttcg ccggccaacc tgtccccgca gccggaggtc tacgggcatt | |
| 781 tcctgggcgt gcgcggcagc tgcattcccc agccgcgccc ggtgcccggc agccagaagg | |
| 841 gcgtgctggt ggcccctgga ggcctggcgc tgccggccta cggcgaggac ggggccctgg | |
| 901 agcacgagcg catgcaacag ctggagcacg gcggcctgca gccaggcctg gtcaaccaca | |
| 961 tggtggtgca gcatggcctg ccgggccccg acagccagcc ggccggcctg ttcaagaccg | |
| 1021 aacgcctgga ggagttcccg ggcagcaccg tagacctacc ccccgcgcct ccgctccctc | |
| 1081 ctctgccgcc gccccaaggc cccccacccc cttaccatgc ccatgcgcac cttcaccacc | |
| 1141 cggagctcgg gccccacgcc cagcagctgg ccttgcccca ggccaccctg gacgacgacg | |
| 1201 gggagatgga cggcatcggg ggcaagcatt gctaccgctg gatcgactgc agcgccctgt | |
| 1261 acgaccagca ggaggagctc gtgcggcaca tcgagaaggt ccacatcgac cagcgcaaag | |
| 1321 gggaggactt cacttgcttc tgggccggtt gccctcgaag atacaagccc ttcaacgccc | |
| 1381 gctataaact gctgatccac atgagagtcc actctgggga gaagcccaac aagtgtacgt | |
| 1441 ttgaaggttg cgagaaggcc ttttcaaggc ttgaaaatct caagatccac ttgcggagcc | |
| 1501 acacaggcga gaagccgtat ttgtgccagc atccgggttg tcagaaggcc ttcagtaact | |
| 1561 ccagtgaccg cgccaaacac cagcggacgc atctggacac caaaccttat gcttgtcaaa | |
| 1621 ttccaggatg taccaaacgc tacacagacc caagttccct aagaaagcat gtgaaggcac | |
| 1681 attcttccaa agagcaacaa gcaaggaaaa agttgcggtc cagcacagag ctccatccag | |
| 1741 acctgctcac agattgcctc accgtgcagt ccctgcagcc ggccacttcc cctagagatg | |
| 1801 ctgctgctga agggaccgtg ggacgctccc ctggacccgg gcctgacctc tattcagctc | |
| 1861 ccattttctc cagcaattat tcaagccgaa gtggaacagc tgctggggcc gtaccacccc | |
| 1921 cacatcctgt cagtcaccct tctccaggac ataatgtaca ggggagccct cacaacccct | |
| 1981 cctcccagtt acctccactc acagctgtgg acgcaggagc tgagaggttt gcaccttctg | |
| 2041 ctccatctcc tcaccacatc agcccccgga gagttccagc tccttcttca atactgcaaa | |
| 2101 gaacacagcc tccctatacc cagcagccat caggctcaca cctgaagtcc tatcagccag | |
| 2161 aaacaaactc ttcttttcaa ccaaatggta tccatgtcca tggattttat gggcagctgc | |
| 2221 agaagttctg tcccccacac taccccgatt cccagagaat tgtgccgcct gtcagctcct | |
| 2281 gcagtgtggt gccttcgttt gaggactgcc tagtccctac atccatgggc caggccagtt | |
| 2341 ttgatgtttt ccacagagcc ttctcgactc actcgggcat tacagtgtat gatttacctt | |
| 2401 caagttcctc gagcctcttc ggggagtctc tccgcagcgg ggctgaagat gctaccttct | |
| 2461 tgcagatcag caccgtggac cgctgtccta gccagctctc ctctgtctac accgaaggct | |
| 2521 aaaagctctc ttggccactc ctgcacaccc aggaccttga tgttgcttgc caccttttgt | |
| 2581 tttcgtactc tcacagactg cttgaggaag gatgtcagcc caatcagcgg gccctgcaga | |
| 2641 gttactgatg ttctttcaga aggcaggact gaatggcaga gctagctttg caggaatcat | |
| 2701 tacagttgct cctttttctc tctggttttc ctggactatg tgaccaggag gactgtcttt | |
| 2761 tcaaagggaa tttagagcct cactctcctg gatacctgtt acttcctggc tgtcaaccct | |
| 2821 taaaggaccc cagtgaaatg atgtgcatat gaaaatgtag tttggggatg actctacctg | |
| 2881 aaaactccaa acagggaaaa agacctcaat tacgagaggc atagtgacct gcctcaccta | |
| 2941 atcctcaccc ttgctgctcc tgatgcaaaa ccaggcacaa caagggaaat ccatgtccaa | |
| 3001 agcatagtgt ggtctcaact cttagaaact ccctgggttt cccttttctc ttagccacat | |
| 3061 gctaaccagt tgcttctcag tgtttctctt caatagaaaa ataaggtttt atatgccaaa | |
| 3121 aaaaaaaaaa aaa | |

An amino acid sequence for the protein (Zinc finger protein GLI2 also known as GLI family zinc finger 2) encoded by the GLI2 gene that is a regulator of T cell exhaustion/dysfunction resistance is available from the UniPROT database at accession no. P10070 or accession number NP_005261, NP_001358200, NP_001361282, or NP_001361283, cDNA NM_005270, NM_030379, NM 030380, NM_030381, or NM_001371271 with gene location at Chr 2: 120.74-120.99 Mb.

```
(SEQ ID NO: 23)
METSASATASEKQEAKSGILEAAGFPDPGKKASPLVVAAAAAAA
VAAQGVPQHLLPPFHAPLPIDMRHQEGRYHYEPHSVHGVHGPPALSGSPVISDISLIR
LSPHPAGPGESPENAPHPYVNPHMEHYLRSVHSSPTLSMISAARGLSPADVAQEHLKE
RGLFGLPAPGTTPSDYYHQMTLVAGHPAPYGDLLMQSGGAASAPHLHDYLNPVDVSRF
SSPRVTPRLSRKRALSISPLSDASLDLQRMIRTSPNSLVAYINNSRSSSAASGSYGHL
SAGALSPAFTEPHPINPVAYQQILSQQRGLGSAFGHTPPLIQPSPTFLAQQPMALTSI
NATPTQLSSSSNCLSDTNQNKQSSESAVSSTVNPVAIHKRSKVKTEPEGLRPASPLAL
TQGQVSGHGSCGCALPLSQEQLADLKEDLDRDDCKQEAEVVIYETNCHWEDCTKEYDT
QEQLVHHINNEHIHGEKKEFVCRWQACTREQKPFKAQYMLVVHMRRHTGEKPHKCTFE
GCSKAYSRLENLKTHLRSHTGEKPYVCEHEGCNKAFSNASDRAKHQNRTHSNEKPYIC
KIPGCTKRYTDPSSLRKHVKTVHGPDAHVTKKQRNDVHLRTPLLKENGDSEAGTEPGG
PESTEASSTSQAVEDCLHVRAIKTESSGLCQSSPGAQSSCSSEPSPLGSAPNNDSGVE
MPGTGPGSLGDLTALDDTPPGADTSALAAPSAGGLQLRKHMTTMHRFEQLKKEKLKSL
KDSCSWAGPTPHTRNTKLPPLPGSGSILENESGSGGGGPAGLLPNPRLSELSASEVTM
LSQLQERRDSSTSTVSSAYTVSRRSSGISPYFSSRRSSEASPLGAGRPHNASSADSYD
PISTDASRRSSEASQCSGGSGLLNLTPAQQYSLRAKYAAATGGPPPTPLPGLERMSLR
TRLALLDAPERTLPAGCPRPLGPRRGSDGPTYGHGHAGAAPAFPHEAPGGGARRASDP
VRRPDALSLPRVQRFHSTHNVNPGPLPPCADRRGLRLQSHPSTDGGLARGAYSPRPPS
ISENVAMEAVAAGVDGAGPEADLGLPEDDLVLPDDVVQYIKAHASGALDEGTGQVYPT
ESTGFSDNPRLPSPGLHGQRRMVAADSNVGPSAPMLGGCQLGFGAPSSLNKNNMPVQW
NEVSSGTVDALASQVKPPPFPQGNLAVVQQKPAFGQYPGYSPQGLQASPGGLDSTQPH
LQPRSGAPSQGIPRVNYMQQLRQPVAGSQC2GMTTTMSPHACYGQVHPQLSPSTISGA
LNQFPQSCSNMPAKPGHLGHPQQTEVAPDPTTMGNRHRELGVPDSALAGVPPPHPVQS
YPQQSHHLAASMSQEGYHQVPSLLPARQPGEMEPQTGPMGVATAGFGLVQPRPPLEPS
PTGRHRGVRAVQQQLAYARATGHAMAAMPSSQETAEAVPKGAMGNMGSVPPQPPPQDA
GGAPDHSMLYYYGQIHMYEQDGGLENLGSCQVMRSQPPQPQACQDSIQPQPLPSPGVN
QVSSTVDSQLLEAPQIDEDAIMDDGDHSSLESGALSPSLLHSLSQNSSRLTTPRNSLT
LPSIPAGISNMAVGDMSSMLTSLAEESKELNMMT
  (SEQ ID NO: 24)
      1 gctgatggat tgcagaagtg ccggcgcttg ccagccgagg cagcacggct ccgcggactt
    61 tttttcaaac tcccatcaat gagacttcga ggaggagcgg gcggcggcgg cggctgcgac
  121 tgcgaacgcg gaggaaggcc aggagccgca ggaggagccg gaggaaagag cttgggccgc
  181 gcggcgcgcc gcagcctcgg ggagccgcct gctcgccggc ggtaggggct gcgcggcgcc
  241 cgcccgcctc taggtccccz ctcttgcctg gcccgccccg ccccggctgg ctggagcccc
  301 ggcacaaggc agccagccga gggtcgccgc gccagccaag gtgggatggg ggcccacagc
  361 caccgcccgg cgcccgagag gccacctgcg tgctagaggc aaacttttgt ctctctcgga
  421 ttgccaccca ggacgatgag cggctgagat ggagacgtct gcctcagcca ctgcctccga
  481 gaagcaagaa gccaaaagtg ggatcctgga ggccgctggc ttccccgacc cgggtaaaaa
  541 ggcctctcct ttggtggtgg ctgcagcggc agcagcagcg gtagctgccc aaggagtgcc
  601 gcagcatctc ttgccaccat tccatgcgcc cctaccgatt gacatgcgac accaggaagg
  661 aaggtaccat tacgagcctc attctgtcca cggtgtgcac gggccccctg ccctcagcgg
  721 cagccctgtc atctctgaca tctccttgat ccggctttcc ccgcacccgg ctggccctgg
  781 ggagtccccc ttcaacgccc cccacccgta cgtgaacccc cacatggagc actacctccg
  841 ttctgtgcac agcagcccca cgctctccat gatctctgca gccaggggcc tcagccccgc
  901 tgatgtggcc caggagcacc ttaaggagag gggactgttt ggccttcctg ctccaggcac
  961 caccccctca gactattacc accagatgac cctcgtggca ggccaccccg cgccctacgg
1021 ggacctgctg atgcagagcg ggggcgctgc cagcgcaccc catctccacg actacctcaa
1081 ccccgtggac gtgtcccgtt tctccagccc gggggtgacg ccccgcctga gccgraagcg
1141 ggcgctgtcc atctccccac tctcagacgc cagcctggac ctgcagcgga tgatccgcac
1201 ctcacccaac tcgctagtgg cctacatcaa caactcccga agcagctcgg cggccagcgg
1261 ttcctacggg catctgtcag cgggtgccct cagcccagcc ttcaccttcc cccaccccat
1321 caaccccgtg gcctaccagc agattctgag ccagcagagg ggtctggggt cagcctttgg
1381 acacacacca cccctgatcc agccctcacc caccttcctg gcccagcagc ccatggccct
1441 cacctccatc aatgccacgc ccacccagct cagcagcagc agcaactgtc tgagtgacac
1501 caaccagaac aagcagagca gtgagtcggc cgtcagcagc accgtcaacc ctgtcgccat
1561 tcacaagcgc agcaaggtca agaccgagcc tgagggcctg cggccggcct cccctctggc
1621 gctgacgcag ggccaggtgt ctggacacgg ctcatgtggg tgtgcccttc ccctctccca
1681 ggagcagctg gctgacctca aggaagatct ggacagggat gactgtaagc aggaggctga
1741 ggtggtcatc tatgagacca actgccactg ggaagactgc accaaggagt acgacaccca
1801 ggagcagctg gtgcatcaca tcaacaacga gcacatccac ggggagaaga aggagtttgt
1861 gtgccgctgg caggcctgca cgcgggagca gaagcccttc aaggcgcagt acatgctggt
1921 ggtgcacatg cggcgacaca cgggcgagaa gccccacaag tgcacgttcg agggctgctc
1981 gaaggcctac tcccgcctgg agaacctgaa gacacacctg cggtcccaca ccggggagaa
2041 gccatatgtg tgtgagcacg agggctgcaa caaagccttc tccaacgcct cggaccgcgc
2101 caagcaccag aatcgcaccc actccaacga gaaaccctac atctgcaaga tcccaggctg
2161 caccaagaga tacacagacc ccagctctct ccggaagcat gtgaaaacgg tccacggccc
2221 agatgcccac gtcaccaaga agcagcgcaa tgacgtgcac ctccgcacac cgctgctcaa
2281 agagaatggg gacagtgagg ccggcacgga gcctggcggc ccagagagca ccgaggccag
2341 cagcaccagc caggccgtgg aggactgcct gcacgtcaga gccatcaaga ccgagagctc
2401 cgggctgtgt cagtccagcc ccggggccca gtcgtcctgc agcagcgagc cctctcctct
2461 gggcagtgcc cccaacaatg acagtggcgt ggagatgccg gggacggggc ccgggagcct
2521 gggagacctg acggcactgg atgacacacc cccaggggcc gacacctcag ccctggctgc
2581 cccctccgct ggtggcctcc agctgcgcaa acacatgacc accatgcacc ggttcgagca
2641 gctcaagaag gagaagctca agtcactcaa ggattcctgc tcatgggccg ggccgactcc
2701 acacacgcgg aacaccaagc tgcctcccct cccgggaagt ggctccatcc tggaaaactt
2761 cagtggcagt gggggcggcg ggcccgcggg gctgctgccg aacccgcggc tgtcggagct
2821 gtccgcgagc gaggtgacca tgctgagcca gctgcaggag cgccgcgaca gctccaccag
2881 cacggtcagc tcggcctaca ccgtgagccg ccgctcctcc ggcatctccc cctacttctc
2941 cagccgccgc tccagcgagg cctcgcccct gggcgccggc cgcccgcaca acgcgagctc
3001 cgctgactcc tacgacccca tctccacgga cgcgtcgcgg cgctcgagcg aggccagcca
3061 gtgcagcggc ggctccgggc tgctcaacct cacgccggcg cagcagtaca gcctgcgggc
3121 caagtacgcg gcagccactg gcggcccccc gcccactccg ctgccgggcc tggagcgcat
3181 gagcctgcgg accaggctgg cgctgctgga cgcgcccgag cgcacgctgc ccgccggctg
3241 cccacgccca ctggggccgc ggcgtggcag cgacgggccg acctatggcc acggccacgc
3301 gggggctgcg cccgccttcc cccacgaggc tccaggcggc ggagccaggc gggccagcga
3361 ccctgtgcgg cggcccgatg ccctgtccct gccgcgggtg cagcgcttcc acagcaccca
3421 caacgtgaac cccggcccgc tgccgccctg tgccgacagg cgaggcctcc gcctgcagag
3481 ccacccgagc accgacggcg gcctggcccg cggcgcctac tcgccccggc cgcctagcat
3541 cagcgagaac gtggcgatgg aggccgtggc ggcaggagtg gacggcgcgg ggcccgaggc
3601 cgacctgggg ctgccggagg acgacctggt gcttccagac gacgtggtgc agtacatcaa
3661 ggcgcacgcc agtggcgctc tggacgaggg caccgggcag gtgtatccca cggaaagcac
3721 tggcttctct gacaacccca gactacccag cccggggctg cacggccagc gcaggatggt
3781 ggctgcggac tccaacgtgg gcccctccgc ccctatgctg ggaggatgcc agttaggctt
3841 tggggcgccc tccagcctga acaaaaataa catgcctgtg cagtggaatg aggtgagctc
3901 cggcaccgta gacgccctgg ccagccaggt gaagcctcca ccctttcctc agggcaacct
3961 ggcggtggtg cagcagaagc ctgcctttgg ccagtacccg ggctacagtc cgcaaggcct
4021 acaggctagc cctgggggcc tggacagcac gcagccacac ctgcagcccc gcagcggagc
4081 cccctcccag ggcatcccca gggtaaacta catgcagcag ctgcgacagc cagtggcagg
4141 cagccagtgt cctggcatga ctaccactat gagcccccat gcctgctatg gccaagtcca
4201 cccccagctg agccccagca ccatcagtgg ggccctcaac cagttccccc aatcctgcag
4261 caacatgcca gccaagccag ggcatctggg gcaccctcag cagacagaag tggcacctga
4321 ccccaccacg atgggcaatc gccacaggga acttggggtc cccgattcag ccctggctgg
4381 agtgccacca cctcacccag tccagagcta cccacagcag agccatcacc tggcagcctc
4441 catgagccag gagggctacc accaggtccc cagccttctg cctgcccgcc agcctggctt
4501 catggagccc caaacaggcc cgatgggggt ggctacagca ggctttggcc tagtgcagcc
4561 ccggcctccc ctcgagccca gccccactgg ccgccaccgt ggggtacgtg ctgtgcagca
4621 gcagctggcc tacgccaggg ccacaggcca tgccatggct gccatgccgt ccagtcagga
4681 aacagcagag gctgtgccca agggagcgat gggcaacatg gggtcggtgc ctccccagcc
4741 gcctccgcag gacgcaggtg gggccccgga ccacagcatg ctctactact acggccagat
4801 ccacatgtac gaacaggatg gaggcctgga gaacctcggg agctgccagg tcatgcggtc
4861 ccagccacca cagccacagg cctgtcagga cagcatccag ccccagccct tgccctcacc
4921 aggggtcaac caggtgtcca gcactgtgga ctcccagctc ctggaggccc cccagattga
1981 cttcgatgcc atcatggatg atggcgatca ctcgagtttg ttctcgggtg ctctgagccc
5041 cagcctcctc cacagcctct cccagaactc ctcccgcctc accacccccc gaaactcctt
5101 gaccctgccc tccatccccg caggcatcag caacatggct gtcggggaca tgagctccat
5161 gctcaccagc ctcgccgagg agagcaagtt cctgaacatg atgacctaga ggcccgagcg
5221 cctggtgctg agtgcacccg gaggggtcat cgctgcccag agcctgggga ttccagctgt
5281 cttgtctttt tccaaaaaag tgttaaatag gcttgagggg ttgttgcgca atggccgctt
5341 cagatgacag atgttgtaag agaaggttta tgggcatcct ctctggtctt ttggattatt
5401 cctcagaaca atgaaaaaag tctccatagg acaggaagga atgcaaaact catttacaca
5461 gtgctttcca gcctttggtg cttacaggac cgcgctgttc cggcttcttc acggctgaca
5521 ttcggctaac gagggattac tttggccaaa acctttcaaa ggatatgcag aaagatggta
5581 gggagcattt gggtttgaat ctgaatgcta tactggatac tctgctccgg aaagatgagc
5641 tttttattct actacttgga aggaaaagga attcctggtc cacctgaatt cctctatgaa
5701 gcctaactct tgaggtctct aacatacctt gtcatagagg aaaagcacag attatacctg
5761 gatgattcag gagcacattc tgattccagg tttggtagag ctggctcttc tactccgtaa
5821 agccgagtct gggactggca gcccatccaa gtgtatatga atgaataaag catccaagta
5881 tatatgaatg aataaagtat gtaagtatca ccagaaaaag gaaagaaaaa atgtactcct
5941 tggggcaagc ccagaagctg ccctggcctc tccagaccgt gtttacagtg tttgcatgta
6001 gaatgtagcc cttcctgaaa agaagacttg tttctaaata cctcggggct gctggagccg
6061 ctgtgggtta gggatggact gaggcctcga ggagtgaggg tgcacccggg gcccagcctc
6121 aggctgccct agggatctct cagtaggaag aggaagttgc gtgtttaccc aatcctgttt
6181 ctccaatgca acgtccaccc actttaccac caaaaactcc agggcctgac ggcagcccgg
6241 tcccccagca ctcaccagca gcccagtgtt ctccaccaag ccacagtgtg catgcctggt
6301 atcctccgga ttcccttcct tctgcccgct gagtcactgg gcagagaatg atgacatgtg
6361 taggtggtgt ggttgggggt ggaaagggga aggggttgat cctcaggact ctgagggagc
6421 atcgttgaat tttcctgttc agtgtgacca agacccacct ggaaatggaa tttggaactg
6481 gcttcaggag acatcattcc tgaacacact gtagggtgaa ttggtgcatc ttccccacca
6541 tacacacaca cacacacaca cacacacaca cacacacaca cacaccccaa accttttcat
6601 ggggaatgtg tggcaacctt gccaaacagc accactcaga gtgtgactct gactgtgacc
6661 ttggccttaa tgaggaactc cttaggagag tttgaggaca aggccaacat cgtcatctgg
6721 gctcgctgcg tcccagcaca tcaaactctg tccagagaca aggccaactg caaatgaaag
6781 ccagggaaca ttgctaaggg tctgtggctc tgtggtggtg ttcatcgcct tcctgagata
6841 ggatttccct tgccagtccc aacctgtata tattctgtac agaagacatc cctgaatata
6901 ctgtaggtga gtcgtccagc caaatttata tctccaaaac atttttagct ttttctacat
6961 gctatgaatt gagatgacat gctcaacttg taaataagtc tttttgtaca ttaaaaaagt
/021 aattttttca taatttatct tgtctatctg cttccccctt gacagtagtt aatgagaacc
7081 tgggcagtaa atttggtgca ttcgagcaga aattaggctg tattttttct taacagtgtc
7141 aaaattgact atcccgcctt tgccaagaaa tgtttaatgc tgaggca
```

An amino acid sequence for the protein (Interferon regulatory factor 4 (IRF4) also known as MUM1) encoded by the IRF4 gene that is a regulator of T cell exhaustion/dysfunction resistance is available from the UniPROT database at accession no. Q15306 or accession number NP_001182215 or NP_002451, cDNA NM_001195286 or NM_002460 with gene location at Chr 6: 0.39-0.41 Mb.

```
  (SEQ ID NO: 25)
MNLEGGGRGGEFGMSAVSCGNGKLRQWLIDQIDSGKYPGLVWEN
EEKSIFRIPWKHAGKQDYNREEDAALFKAWALFKGKFREGIDKPDPPTWKTRLRCALN
KSNDFEELVERSQLDISDPYKVYRIVPEGAKKGAKQLTLEDPQMSMSHPYTMTTPYPS
LPAQQVHNYMMPPLDRSWRDYVPDQPHPEIPYQCPMTFGPRGHHWQGPACENGCQVTG
TFYACAPPESQAPGVPTEPSIRSAEALAFSDCRLHICLYYREILVKELTTSSPEGCRI
SHGHTYDASNLDQVLFPYPEDNGQRKNIEKLLSHLERGVVLWMAPDGLYAKRLCQSRI
YWDGPLALCNDRPNKLERDQTCKLEDTQQFLSELQAFAHHGRSLPRFQVTLCFGEEFP
DPQRQRKLITAHVEPLLARQLYYFAQQNSGHFLRGYDLPEHISNPEDYHRSIRHSSIQ
E
  (SEQ ID NO: 26)
      1 agtttcaccg ctcgatcttg ggacccaccg ctgccctcag ctccgagtcc agggcgagtg
    61 cagagcagag cgggcggagg accccgggcg cgggcgcgga cggcacgcgg ggcatgaacc
  121 tggagggcgg cggccgaggc ggagagttcg gcatgagcgc ggtgagctgc ggcaacggga
  181 agctccgcca gtggctgatc gaccagatcg acagcggcaa gtaccccggg ctggtgtggg
  241 agaacgagga gaagagcatc ttccgcatcc cctggaagca cgcgggcaag caggactaca
  301 accgcgagga ggacgccgcg ctcttcaagg cttgggcact gtttaaagga aagttccgag
  361 aaggcatcga caagccggac cctcccacct ggaagacgcg cctgcggtgc gctttgaaca
  421 agagcaatga ctttgaggaa ctggttgagc ggagccagct ggacatctca gacccgtaca
  481 aagtgtacag gattgttcct gagggagcca aaaaaggagc caagcagctc accctggagg
  541 acccgcagat gtccatgagc cacccctaca ccatgacaac gccttaccct tcgctcccag
  601 cccagcaggt tcacaactac atgatgccac ccctcgaccg aagctggagg gactacgtcc
  661 cggatcagcc acacccggaa atcccgtacc aatgtcccat gacgtttgga ccccgcggcc
  721 accactggca aggcccagct tgtgaaaatg gttgccaggt gacaggaacc ttttatgctt
  781 gtgccccacc tgagtcccag gctcccggag tccccacaga gccaagcata aggtctgccg
  841 aagccttggc gttctcagac tgccggctgc acatctgcct gtactaccgg gaaatcctcg
  901 tgaaggagct gaccacgtcc agccccgagg gctgccggat ctcccatgga catacgtatg
  961 acgccagcaa cctggaccag gtcctgttcc cctacccaga ggacaatggc cagaggaaaa
1021 acattgagaa gctgctgagc cacctggaga ggggcgtggt cctctggatg gcccccgacg
1081 ggctctatgc gaaaagactg tgccagagca ggatctactg ggacgggccc ctggcgctgt
1141 gcaacgaccg gcccaacaaa ctggagagag accagacctg caagctcttt gacacacagc
1201 agttcttgtc agagctgcaa gcgtttgctc accacggccg ctccctgcca agattccagg
1261 tgactctatg ctttggagag gagtttccag accctcagag gcaaagaaag ctcatcacag
1321 ctcacgtaga acctctgcta gccagacaac tatattattt tgctcaacaa aacagtggac
1381 atttcctgag gggctacgat ttaccagaac acatcagcaa tccagaagat taccacagat
1441 ctatccgcca ttcctctatt caagaatgaa aaatgtcaag atgagtggtt ttctttttcc
1501 tttttttttt tttttttttg atacggggat acggggtctt gctctgtctc ccaggctgga
1561 gtgcagtgac acaatctcag ctcactgtga cctccgcctc ctgggttcaa gagactctcc
1621 tgcctcagcc tccctggtag ctgggattac aggtgtgagc cactgcaccc acccaagaca
1681 agtgattttc attgtaaata tttgacttta gtgaaagcgt ccaattgact gccctcttac
1741 tgttttgagg aattcagaag tggagatttc agttcagcgg ttgaggagaa ttgcggcgag
1801 acaagcatgg aaaatcagtg acatctgatt ggcagatgag cttatttcaa aaggaagggt
1861 ggctttgcat ttcttgtgtt ctgtagactg ccatcattga thatcactgt gaaaattgac
1921 caagtgatgt gtttacattt actgaaatgc gctccttaat ttgttgtaga ttaggtcttg
1981 ctggaagaca gagaaaactt gcctttcagt attgacactg actagagtga tgactgcttg
2041 taggtatgtc tgtgccatt: ctcagggaag taagatgtaa attgaagaag cctcacacgt
2101 aaaagaaatg tattaatgta tgtaggagct gcagttcttg tggaagacac ttgctgagtg
2161 aaggaaatga atctttgact gaagccgtgc ctgtagcctt ggggaggccc atcccccacc
2221 tgccagcggt ttcctggtgt gggtccctct gccccgccct ccttcccatt ggctttctct
2281 ccttggcctt tcctggaagc cagttagtaa acttcctatt ttcttgagtc aaaaaacatg
2341 agcgctactc ttggatggga catttttgtc tgtcctacaa tctagtaatg tctaagtaat
2401 ggttaagttt tcttgtttct gcatcttttt gaccctcatt ctttagagat gctaaaattc
2461 ttcgcataaa gaagaagaaa ttaaggaaca taaatcttaa tacttgaact gttgcccttc
2521 tgtccaagta cttaactatc tgttcccttc ctctgtgcca cgctcctctg tttgtttggc
2581 tgtccagcga tcagccatgg cgacactaaa ggaggaggag ccggggactc ccaggctgga
2641 gagcactgcc aggacccacc actggaagca ggatggagct gactacggaa ctgcacactc
2701 agtgggctgt ttctgcttat ttcatctgtt ctatgcttcc tcgtgccaat tatagtttga
2761 cagggcctta aaattacttg gctttttcca aatgcttcta tttatagaat cccaaagacc
2821 tccacttgct taagtatacc tatcacttac atttttgtgg ttttgagaaa gtacagcagt
2881 agactggggc gtcacctcca ggccgtttct catactacag gatatttact attactccca
2941 ggatcagcag aagattgcgt agctctcaaa tgtgtgttcc tgcttttcta atggatattt
3001 taaattcatt caacaagcac ctagtaagtg cctgctgtat ccctacatta cacagttcag
3061 cctttatcaa gcttagtgag cagtgagcac tgaaacatta ttttttaatg tttaaaaagt
3121 ttctaatatt aaagtcagaa tattaataca attaatatta atattaacta cagaaaagac
3181 aaacagtaga gaacagcaaa aaaataaaaa ggatctcctt ttttcccagc ccaaattctc
3241 ctctctaaaa gtgtccacaa gaaggggtgt ttattcttcc aacacatttc acttttctgt
3301 aaatatacat aaacttaaaa agaaaacctc atggagtcat cttgcacaca ctttcatgca
3361 gtgctctttg tagctaacag tgaagattta cctcgttctg ctcagaggcc ttgctgtgga
3421 gctccactgc catgtaccca gtagggtttg acatttcatt agccatgcaa catggatatg
3481 tattgggcag cagactgtgt ttcgtgaact gcag gatgt atacatctta tagatgcaaa
3541 gtattttggg gtatattatc ctaagggaag ataaagatga tattaagaac tgctgtttca
3601 cggggccctt acctgtgacc ctctttgctg aagaatattt aaccccacac agcacttcaa
3661 agaagctgtc ttggaagtct gtctcaggag caccctgtct tcttaattct ccaagcggat
3721 gctccatttc aattgctttg tgacttcttc ttctttgttt ttttaaatat tatgctgctt
3781 taacagtgga gctgaatttt ctggaaaatg cttcttggct ggggccacta cctcctttcc
3841 tatctttaca tctatgtgta tgttgacttt ttaaaattct gagtgatcca gggtatgacc
3901 tagggaatga actagctatg aaatactcag ggttaggaat cctagcactt gtctcaggac
3961 tctgaaaagg aacggcttcc tcattccttg tcttgataaa gtggaattgg caaactagaa
4021 tttagtttgt actcagtgga cagtgctgtt gaagatttga ggacttgtta aagagcactg
4081 ggtcatatgg aaaaaatgta tgtgtctccc aggtgcattt cttggtttat gtcttgttct
4141 tgagattttg tatatttagg aaaacctcaa gcagtaatta atatctcctg gaacactata
4201 gagaaccaag tgaccgactc atttacaact gaaacctagg aagcccctga gtcctgagcg
4261 aaaacaggag agttagtcgc cctacagaaa acccagctag actattgggt atgaactaaa
4321 aagagactgt gccatggtga gaaaaatgta aaatcctaca gtgaaatgag cagcccttac
4381 agtattgtta ccaccaaggg caggtaggta ttagtgtttg aaaaagctgg tctttgagcg
4441 agggcataaa tacagctagc cccaggggtg gaacaactct gggagtcttg ggtactcgca
4501 cctcttggct ttgttgatgc tccgccagga aggccacttg tgtgtgcgtg tcagttactt
4561 ttttagtaac aattcagatc cagtgtaaac ttccgttcat tgctctccag tcacatgccc
4621 ccacttcccc acaggtgaaa gtttttctga aagtgttggg attggttaag gtctttattt
4681 gtattacgta tctccccaag tcctctgtgg ccagctgcat ctgtctgaat ggtgcgtgaa
4741 ggctctcaga ccttacacac cattttgtaa gttatgtttt acatgccccg tttttgagac
4801 tgatctcgat gcaggtggat ctccttgaga tcctgatagc ctgttacagg aatgaagtaa
4861 aggtcagttt ttttttgtat tgattttcac agctttgagg aacatgcata agaaatgtag
4921 ctgaagtaga ggggacgtga gagaagggcc aggccggcag gccaaccctc ctccaatgga
4981 aattcccgtg ttgcttcaaa ctgagacaga tgggacttaa caggcaatgg ggtccacttc
5041 cccctcttca gcatcccccg taccccactt tctgctgaaa gaactgccag caggtaggac
5101 cccagaggcc cccaaatgaa agcttgaatt tcccctactg gctctgcgtt ttgctgagat
5161 ctgtaggaaa ggatgcttca caaactgagg tagataatgc tatgctgtcg ttggtataca
5221 tcatgaattt ttatgtaaat tgctctgcaa agcaaattga tatgtttgat aaatttatgt
5281 ttttaggtaa ataaaaactt ttaaaaattt gtta
```

An amino acid sequence for the protein (Interleukin-2 receptor alpha chain (also called TAC antigen, P55 and mainly CD25)) encoded by the IL2RA gene that is a regulator of T cell exhaustion/dysfunction resistance is available from the UniPROT database at accession no. P01589 or accession number NP 000408, NP_001295171 or NP_001295172, cDNA NM_000417, NM_001308242 or NM_001308243 with gene location at Chr 10: 6.01-6.06 Mb.

```
(SEQ ID NO: 27)
MDSYLLMWGLLTFIMVPGCQAELCDDDPPEIPHATFKAMAYKEG
TMLNCECKRGERRIKSGSLYMLCTGNSSHSSWDNQCQCTSSATRNTTKQVTPQPEEQK
ERKTTEMQSPMQPVDQASLPGHCREPPPWENEATERIYHFVVGQMVYYQCVQGYRALH
RGPAESVCKMTHGKTRWTQPQLICTGEMETSQFPGEEKPQASPEGRPESETSCLVTTT
DFQIQTEMAATMETSIFTTEYQVAVAGCVFLLISVLLLSGLTWQRRQRKSRRTI
  (SEQ ID NO: 28)
      1 agtttcctgg ctgaacacgc cagcccaata cttaaagaga gcaactcctg actccgatag
    61 agactggatg gacccacaag ggtgacagcc caggcggacc gatcttccca tcccacatcc
  121 tccggcgcga tgccaaaaag aggctgacgg caactgggcc ttctgcagag aaagacctcc
  181 gcttcactgc cccggctggt cccaagggtc aggaagatgg attcatacct gctgatgtgg
  241 ggactgctca cgttcatcat ggtgcctggc tgccaggcag agctctgtga cgatgacccg
  301 ccagagatcc cacacgccac attcaaagcc atggcctaca aggaaggaac catgttgaac
  361 tgtgaatgca agagaggttt ccgcagaata aaaagcgggt cactctatat gctctgtaca
  421 ggaaactcta gccactcgtc ctgggacaac caatgtcaat gcacaagctc tgccactcgg
  481 aacacaacga aacaagtgac acctcaacct gaagaacaga aagaaaggaa aaccacagaa
  541 atgcaaagtc caatgcagcc agtggaccaa gcgagccttc caggtcactg cagggaacct
  601 ccaccatggg aaaatgaagc cacagagaga atttatcatt tcgtggtggg gcagatggtt
  661 tattatcagt gcgtccaggg atacagggct ctacacagag gtcctgctga gagcgtctgc
  721 aaaatgaccc acgggaagac aaggtggacc cagccccagc tcatatgcac aggtgaaatg
  781 gagaccagtc agtttccagg tgaagagaag cctcaggcaa gccccgaagg ccgtcctgag
  841 agtgagactt cctgcctcgt cacaacaaca gattutcaaa tacagacaga aatggctgca
  901 accatggaga cgtccatatc tacaacagag taccaggtag cagtggccgg ctgtgttttc
  961 ctgctgatca gcgtcctcct cctgagtggg ctcacctggc agcggagaca gaggaagagt
1021 agaagaacaa tctagaaaac caaaagaaca agaatttctt ggtaagaagc cgggaacaga
1081 caacagaagt catgaagccc aagtgaaatc aaaggtgcta aatggtcgcc caggagacat
1141 ccgttgtgct tgcctgcgtt ttggaagctc tgaagtcaca tcacaggaca cggggcagtg
1201 gcaaccttgt ctctatgcca gctcagtccc atcagagagc gagcgctacc cacttctaaa
1261 tagcaatttc gccgttgaag aggaagggca aaaccactag aactctccat cttattttca
1321 tgtatatgtg ttcattaaag catgaatggt atggaactct ctccacccta tatgtagtat
1381 aaagaaaagt aggtttacat tcatctcatt ccaacttccc agttcaggag tcccaaggaa
1441 agccccagca ctaacgtaaa tacacaacac acacactcta ccctatacaa ctggacattg
1501 tctgcgtggt tcctttctca gccgcttctg actgctgatt ctcccgttca cgttgcctaa
1561 taaacatcct tcaagaactc tgggctgcta cccagaaatc attttaccct tggctcaatc
1621 ctctaagcta acccccttct actgagcctt cagtcttgaa tttctaaaaa acagaggcca
1681 tggcagaata atctttgggt aacttcaaaa cggggcagcc aaacccatga ggcaatgtca
1741 ggaacagaag gatgaatgag gtcccaggca gagaatcata cttagcaaag ttttacctgt
1801 gcgttactaa ttggcctctt taagagttag ttttttggg attgctatga atgataccct
1861 gaatttggcc tgcactaatt tgatctttac aggggacac acaaggtgca aatcaatgcg
1921 tacgtttcct gagaagtgtc taaaaacacc aaaaagggat ccgtacattc aatgtttatg
1981 caaggaagga aagaaagaag gaagtgaaga gggagaaggg atggaggtca cactggtaga
2041 acgtaaccac ggaaaagagc gcatcaggcc tggcacggtg gctcaggcct ataaccccag
2101 ctccctagga gaccaaggcg ggagcatctc ttgaggccag gagtttgaga ccagcctggg
2161 cagcatagca agacacatcc ctacaaaaaa ttagaaattg gctggatgtg gtggcatacg
2221 cctgtagtcc tagccactca ggaggctgag gcaggaggat tgcttgagcc caggagttcg
2281 aggctgcagt cagtcatgat ggcaccactg cactccagcc tgggcaacag agcaagatcc
2341 tgtctttaag gaaaaaaaga caagatgagc ataccagcag tccttgaaca ttatcaaaaa
2401 gttcagcata ttagaatcac cgggaggcct tgttaaaaga gttcgctggg cccatcttca
2461 gagtctctga gttgttggtc tggaatagag ccaaatgttt tgtgtgtcta acaattccca
2521 ggtgctgttg ctgctgctac tattccagga acacactttg agaaccattg tgttattgct
2581 ctgcacgccc acccactctc aactcccacg aaaaaaatca acttccagag ctaagatttc
2641 ggtggaagtc ctggttccat atctggtgca agatctcccc tcacgaatca gttgagtcaa
2701 cattctagct caacaacatc acacgattaa cattaacgaa aattattcat ttgggaaact
2761 atcagccagt tttcacttct gaaggggcag gagagtgtta tgagaaatca cggcagtttt
2821 cagcagggtc cagattcaga ttaaataact attttctgtc atttctgtga ccaaccacat
2881 acaaacagac tcatctgtgc actctccccc tcccccttca ggtatatgtt ttctgagtaa
2941 agttgaaaag aatctcagac cagaaaatat agatatatat ttaaatctta cttgagtaga
3001 actgattacg acttttgggt gttgaggggt ctataagatc aaaacttttc catgataata
3061 ctaagatgtt atcgaccatt tatctgtcct tctctcaaaa gtgtatggtg gaattttcca
3121 gaagctatgt gatacgtgat gatgtcatca ctctgctgtt aacatataat aaatttattg
3181 ctattgttta taaaagaata aatgatattt tttaaaaa
```

An amino acid sequence for the protein (Src homology 2 (SH2) domain containing inositol polyphosphate 5-phosphatase 1 (SHIP1)) encoded by the INPP5D gene that is a regulator of T cell exhaustion/dysfunction resistance is available from the UniPROT database at accession no. Q92835 or accession number NP_001017915 or NP_005532, cDNA NM_001017915 or NM_005541 with gene location at Chr 2: 233.06-233.21 Mb.

```
  (SEQ ID NO: 29)
MVPCWNHGNITRSKAEELLSRTGKDGSELVRASESISRAYALCV
LYRNCVYTYRILPNEDDKFTVQASEGVSMRFFTKLDQLIEFYKKENMGLVTHLQYPVP
LEEEDTGDDPEEDTVESVVSPPELPPRNIPLTASSCEAKEVPFSNENPRATETSRPSL
SETLFQRLQSMDTSGLPEEHLKAIQDYLSTQLAQDSEFVKTGSSSLPHLKKLTTLLCK
ELYGEVIRTLPSLESLQRLFDQQLSPGLRPRPQVPGEANPINMVSKLSQLTSLLSSIE
DKVKALLHEGPESPHRPSLIPPVTFEVKAESLGIPQKMQLKVDVESGKLIIKKSKDGS
EDKFYSHKKILQLIKSQKFLNKLVILVETEKEKILRKEYVFADSKKREGFCQLLQQMK
NKHSEQPEPDMITIFIGTWNMGNAPPPKKITSWFLSKGQGKTRDDSADYIPHDIYVIG
TQEDPLSEKEWLEILKHSLQEITSVTFKTVAIHTLWNIRIVVLAKPEHENRISHICTD
NVKTGIANTLGNKGAVGVSFMENGTSLGFVNSHLTSGSEKKLRRNQNYMNILRFLALG
DKKLSPFNITHRFTHLFWFGDLNYRVDLPTWEAETIIQKIKQQQYADLLSHDQLLTER
REQKVFLHFEEEEITFAPTYRFERLTRDKYAYTKQKATGMKYNLPSWCDRVLWKSYPL
VHVVCQSYGSTSDIMTSDHSPVFATFEAGVTSQFVSKNGPGTVDSQGQIEFLRCYATL
KTKSQTKFYLEFHSSCLESFVKSQEGENEEGSEGELVVKFGETLPKLKPIISDPEYLL
DQHILISIKSSDSDESYGEGCIALRLEATETQLPIYTPLTHHGELTGHFQGEIKLQTS
QGKTREKLYDFVKTERDESSGPKTLKSLTSHDPMKQWEVTSRAPPCSGSSITEIINPN
YMGVGPFGPPMPLHVKQTLSPDQQPTAWSYDQPPKDSPLGPCRGESPPTPPGQPPISP
KKFLPSTANRGLPPRTQESRPSDLGKNAGDTLPQEDLPLTKPEMFENPLYGSLSSFPK
PAPRKDQESPKMPRKEPPPCPEPGILSPSIVLTKAQEADRGEGPGKQVPAPRLRSFTC
SSSAEGRAAGGDKSQGKPKTPVSSQAPVPAKRPIKPSRSEINQQTPPTPTPRPPLPVK
SPAVLHLQHSKGRDYRDNTELPHHGKHRPEEGPPGPLGRTAMQ
  (SEQ ID NO: 30)
      1 agttaagctg gtggcagcag ccgaggccac caagaggcaa cgggcggcag gctgcagtgg
    61 aggggcctcc gctcccctcg gtggtgtgtg ggtcctgggg gtgcctgccg gcccggccga
  121 ggaggcccac gcccaccatg gtcccctgct ggaaccatgg caacatcacc cgctccaagg
  181 cggaggagct gctttccagg acaggcaagg acgggagctt cctcgtgcgt gccagcgagt
  241 ccatctcccg ggcatacgcg ctctgcgtgc tgtatcggaa ttgcgtttac acttacagaa
  301 ttctgcccaa tgaagatgat aaattcactg ttcaggcatc cgaaggcgtc tccatgaggt
  361 tcttcaccaa gctggaccag ctcatcgagt tttacaagaa ggaaaacatg gggctggtga
  421 cccatctgca ataccctgtg ccgctggagg aagaggacac aggcgacgac cctgaggagg
  481 acacagtaga aagtgtcgtg tctccacccg agctgccccc aagaaacatc ccgctgactg
  541 ccagctcctg tgaggccaag gaggttcctt tttcaaacga gaatccccga gcgaccgaga
  601 ccagccggcc gagcctctcc gagacattgt tccagcgact gcaaagcatg gacaccagtg
  661 ggcttccaga agagcatctt aaggccatcc aagattattt aagcactcag ctcgcccagg
  721 actctgaatt tgtgaagaca gggtccagca gtcttcctca cctgaagaaa ctgaccacac
  781 tgctctgcaa ggagctctat ggagaagtca tccggaccct cccatccctg gagtctctgc
  841 agaggttatt tgaccagcag ctctccccgg gcctccgtcc acgtcctcag gttcctggtg
  901 aggccaatcc catcaacatg gtgtccaagc tcagccaact gacaagcctg ttgtcgtcca
  961 ttgaagacaa ggtcaaggcc ttgctgcacg agggtcctga gtctccgcac cggccctccc
1021 ttatccctcc agtcaccttt gaggtgaagg cagagtctct ggggattcct cagaaaatgc
1081 agctcaaagt cgacgttgag tctgggaaac tgatcattaa gaagtccaag gatggttctg
1141 aggacaagtt ctacagccac aagaaaatcc tgcagctcat taagtcacag aaatttctga
1201 ataagttggt gatcttggtg gaaacagaga aggagaagat cctgcggaag gaatatgttt
1261 ttgctgactc caaaaagaga gaaggcttct gccagctcct gcagcagatg aagaacaagc
1321 actcagagca gccggagccc gacatgatca ccatcttcat cggcacctgg aacatgggta
1381 acgccccccc tcccaagaag atcacgtcct ggtttctctc caaggggcag ggaaagacgc
1441 gggacgactc tgcggactac atcccccatg acatttacgt gatcggcacc caagaggacc
1501 ccctgagtga gaaggagtgg ctggagatcc tcaaacactc cctgcaagaa atcaccagtg
1561 tgacttttaa aacagtcgcc atccacacgc tctggaacat ccgcatcgtg gtgctggcca
1621 agcctgagca cgagaaccgg atcagccaca tctgtactga caacgtgaag acaggcattg
1681 caaacacact ggggaacaag ggagccgtgg gggtgtcgtt catgttcaat ggaacctcct
1741 tagggttcgt caacagccac ttgacttcag gaag gaaaa gaaactcagg cgaaaccaaa
1801 actatatgaa cattctccgg ttcctggccc tgggcgacaa gaagctgagt ccctttaaca
1861 tcactcaccg cttcacgcac ctcttctggt ttggggatct taactaccgt gtggatctgc
1921 ctacctggga ggcagaaacc atcatccaga aaatcaagca gcagcagtac gcagacctcc
1981 tgtcccacga ccagctgctc acagagagga gggagcagaa ggtcttccta cacttcgagg
2041 aggaagaaat cacgtttgcc ccaacctacc gttttgagag actgactcgg gacaaatacg
2101 cctacaccaa gcagaaagcg acagggatga agtacaactt gccttcctgg tgtgaccgag
2161 tcctctggaa gtcttatccc ctggtgcacg tggtgtgtca gtcttatggc agtaccagcg
2221 acatcatgac gagtgaccac agccctgtct ttgccacatt tgaggcagga gtcacttccc
2281 agtttgtctc caagaacggc cccgggactg ttgacagcca aggacagatt gagtttctca
2341 ggtgctatgc cacattgaag accaagtccc agaccaaatt ctacctggag ttccactcga
2401 gctgcttgga gagttttgtc aagagtcagg aaggagaaaa tgaagaagga agtgaggggg
2461 agctggtggt gaagtttggt gagactcttc caaagctgaa gcccattatc tctgaccctg
2521 agtacctgct agaccagcac atcctcatca gcatcaagtc ctctgacagc gacgaatcct
2581 atggcgaggg ctgcattgcc cttcggttag aggccacaga aacgcagctg cccatctaca
2641 cgcctctcac ccaccatggg gagttgacag gccacttcca gggggagatc aagctgcaga
2701 cctctcaggg caagacgagg gagaagctct atgactttgt gaagacggag cgtgatgaat
2761 ccagtgggcc aaagaccctg aagagcctca ccagccacga ccccatgaag cagtgggaag
2821 tcactagcag ggcccctccg tgcagtggct ccagcatcac tgaaatcatc aaccccaact
2881 acatgggagt ggggcccttt gggccaccaa tgcccctgca cgtgaagcag accttgtccc
2941 ctgaccagca gcccacagcc tggagctacg accagccgcc caaggactcc ccgctggggc
3001 cctgcagggg agaaagtccc ccgacacctc ccggccagcc gcccatatca cccaagaagt
3061 ttttaccctc aacagcaaac cggggtctcc ctcccaggac acaggagtca aggcccagtg
3121 acctggggaa gaacgcaggg gacacgctgc ctcaggagga cctgccgctg acgaagcccg
3181 agatgtttga gaaccccctg tatgggtccc tgagttcctt ccctaagcct gctcccagga
3241 aggaccagga atcccccaaa atgccgcgga aggaaccccc gccctgcccg gaacccggca
3301 tcttgtcgcc cagcatcgtg ctcaccaaag cccaggaggc tgatcgcggc gaggggcccg
3361 gcaagcaggt gcccgcgccc cggctgcgct ccttcacgtg ctcatcctct gccgagggca
3421 gggcggccgg cggggacaag agccaaggga agcccaagac cccggtcagc tcccaggccc
3481 cggtgccggc caagaggccc atcaagcctt ccagatcgga aatcaaccag cagaccccgc
3541 ccaccccgac gccgcggccg ccgctgccag tcaagagccc ggcggtgctg cacctccagc
3601 actccaaggg ccgcgactac cgcgacaaca ccgagctccc gcatcacggc aagcaccggc
3661 cggaggaggg gccaccaggg cctctaggca ggactgccat gcagtgaagc cctcagtgag
3721 ctgccactga gtcgggagcc cagaggaacg gcgtgaagcc actggaccct ctcccgggac
3781 ctcctgctgg ctcctcctgc ccagcttcct atgcaaggct ttgtgttttc aggaaagggc
3841 ctagcttctg tgtggcccac agagttcact gcctgtgaga cttagcacca agtgctgagg
3901 ctggaagaaa aacgcacacc agacgggcaa caaacagtct gggtccccag ctcgctcttg
3961 gtacttggga ccccagtgcc tcgttgaggg cgccattctg aagaaaggaa ctgcagcgcc
4021 gatttgaggg tggagatata gataataata atattaataa taataatggc cacatggatc
4081 gaacactcat gatgtgccaa gtgctgtgct aagtgcttta cgaacattcg tcatatcagg
4141 atgacctcga gagctgaggc tctagccacc taaaaccacg tgcccaaacc caccagttta
4201 aaacggtgtg tgttcggagg ggtgaaagca ttaagaagcc cagtgccctc ctggagtgag
4261 acaagggctc ggccttaagg agctgaagag tctgggtagc ttgtttaggg tacaagaagc
1321 ctgttctgtc cagcttcagt gacacaagct gctttagcta aagtcccgcg ggttccggca
4381 tggctaggct gagagcaggg atctacctgg cttctcagtt ctttggttgg aaggagcagg
4441 aaatcagctc ctattctcca gtggagagat ctggcctcag cttgggctag agatgccaag
4501 gcctgtgcca ggttccctgt gccctcctcg aggtgggcag ccatcaccag ccacagttaa
4561 gccaagcccc ccaacatgta ttccatcgtg ctggtagaag agtctttgct gttgctcccg
4621 aaagccgtgc tctccagcct ggctgccagg gagggtgggc ctcttggttc caggctcttg
4681 aaatagtgca gccttttct: cctatctctg tggccttcag ctctgcttcc ttggttatta
4741 ggagaataga tgggtgatgt ctttccttat gttgcttttt caacatagca gaattaatgt
4801 agggagctaa atccagtggt gtgtgtgaat gcagaaggga atgcacccca cattcccatg
1861 atggaagtct gcgtaaccaa taaattgtgc ctttctcact ca
```

An amino acid sequence for the protein (Interleukin-2 receptor subunit beta) encoded by the IL2RB gene that is a regulator of T cell exhaustion/dysfunction resistance is available from the UniPROT database at accession no. P14784 or accession number NP_000869, NP_001333151, or NP_001333152, cDNA NM_000878. NM_001346222 or NM_001346223 with gene location at Chr 22: 37.13-37.18 Mb.

```
  (SEQ ID NO: 31)
MAAPALSWRLPLLILLLPLATSWASAAVNGTSQFTCFYNSRANI
SCVWSQDGALQDTSCQVHAWPDRRRWNQTCELLPVSQASWACNLILGAPDSQKLTTVD
IVTLRVLCREGVRWRVMAIQDFKPFENLRLMAPISLQVVHVETHRCNISWEISQASHY
FERHLEFEARTLSPGHTWEEAPLLTLKQKQEWICLETLTPDTQYEFQVRVKPLQGEFT
TWSPWSQPLAFRTKPAALGKDTIPWLGHLLVGLSGAFGFIILVYLLINCRNTGPWLKK
VLKCNTPDPSKFFSQLSSEHGGDVQKWLSSPFPSSSFSPGGLAPEISPLEVLERDKVT
QLLLQQDKVPEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPPSTAPGGSGA
GEERMPPSLQERVPRDWDPQPLGPPTPGVPDLVDFQPPPELVLREAGEEVPDAGPREG
VSFPWSRPPGQGEFRALNARLPLNTDAYLSLQELQGQDPTHLV
  (SEQ ID NO: 32)
      1 agagctcagc agggccctgg agagatggcc acggtcccag caccggggag gactggagag
    61 cgcgcgctgc caccgcccca tgtctcagcc agggcttcct tcctcggctc caccctgtgg
  121 atgtaatggc ggcccctgct ctgtcctggc gtctgcccct cctcatcctc ctcctgcccc
  181 tggctacctc ttgggcatch gcagcggtga atggcacttc ccagttcaca tgcttctaca
  241 actcgagagc caacatctcc tgtgtctgga gccaagatgg ggctctgcag gacacttcct
  301 gccaagtcca tgcctggccg gacagacggc ggtggaacca aacctgtgag ctgctccccg
  361 tgagtcaagc atcctgggcc tgcaacctga tcctcggagc cccagattct cagaaactga
  421 ccacagttga catcgtcacc ctgagggtgc tgtgccgtga gggggtgcga tggagggtga
  481 tggccatcca ggacttcaag ccctttgaga accttcgcct gatggccccc atctccctcc
  541 aagttgtcca cgtggagacc cacagatgca acataagctg ggaaatctcc caagcctccc
  601 actactttga aagacacctg gagttcgagg cccggacgct gtccccaggc cacacctggg
  661 aggaggcccc cctgctgact ctcaagcaga agcaggaatg gatctgcctg gagacgctca
  721 ccccagacac ccagtatgag tttcaggtgc gggtcaagcc tctgcaaggc gagttcacga
  781 cctggagccc ctggagccag cccctggcct tcaggacaaa gcctgcagcc cttgggaagg
  841 acaccattcc gtggctcggc cacctcctcg tgggcctcag cggggctttt ggcttcatca
  901 tcttagtgta cttgctgatc aactgcagga acaccgggcc atggctgaag aaggtcctga
  961 agtgtaacac cccagacccc tcgaagttct tttcccagct gagctcagag catggaggag
1021 acgtccagaa gtggctctct tcgcccttcc cctcatcgtc cttcagccct ggcggcctgg
1081 cacctgagat ctcgccacta gaagtgctgg agagggacaa ggtgacgcag ctgctcctgc
1141 agcaggacaa ggtgcctgag cccgcatcct taagcagcaa ccactcgctg accagctgct
1201 tcaccaacca gggttacttc ttcttccacc tcccggatgc cttggagata gaggcctgcc
1261 aggtgtactt tacttacgac ccctactcag aggaagaccc tgatgagggt gtggccgggg
1321 cacccacagg gtcttccccc caacccctgc agcctctgtc aggggaggac gacgcctact
1381 gcaccttccc ctccagggat gacctgctgc tcttctcccc cagtctcctc ggtggcccca
1441 gccccccaag cactgcccct gggggcagtg gggccggtga agagaggatg cccccttctt
1501 tccaagaaag agtccccaga gactgggacc cccagcccct ggggcctccc accccaggag
1561 tcccagacct ggtggatttt cagccacccc ctgagctggt gctgcgagag gctggggagg
1621 aggtccctga cgctggcccc agggagggag tcagcttccc ctggtccagg cctcctgggc
1681 agggggagtt cagggccctt aatgctcgcc tgcccctgaa cactgatgcc tacttgtccc
1741 tccaagaact ccagggtcag gacccaactc acttggtgta gacagatggc cagggtggga
1801 ggcaggcagc tgcctgctcz gcgccgagcc tcagaaggac cctgttgagg gtcctcagtc
1861 cactgctgag gacactcagt gtccagttgc agctggactt ctccacccgg atggccccca
1921 cccagtcctg cacacttggt ccatccattt ccaaacctcc actgctgctc ccgggtcctg
1981 ctgcccgagc caggaactgt gtgtgttgca ggggggcagt aactccccaa ctccctcgtt
2041 aatcacagga tcccacgaat ttaggctcag aagcatcgct cctctccagc cctgcagcta
2101 ttcaccaata tcagtcctcg cggctctcca gggctccctg ccctgacctc ttccctgggt
2161 tttctgcccc agcctcctcc ttccctcccc tccccgtcca cagggcagcc tgagcgtgct
2221 ttccaaaacc caaatatggc cacgctcccc ctcggttcaa aaccttgcac aggtcccact
2281 gccctcagcc ccacttctca gcctggtact tgtacctccg gtgtcgtgtg gggacatccc
2341 cttctgcaat cctccctacc gtcctcctga gccactcaga gctccctcac accccctctg
2401 ttgcacatgc tattccctgg ggctgctgtg cgctccccct catctaggtg acaaacttcc
2461 ctgactcttc aagtgccggt tttgcttctc ctggagggaa gcactgcctc ccttaatctg
2521 ccagaaactt ctagcgtcag tgctggaggg agaagctgtc agggacccag ggcgcctgga
2581 gaaagaggcc ctgttacta: tcctttggga tctctgaggc ctcagagtgc ttggctgctg
2641 tatctttaat gctggggccc aagtaagggc acagatcccc ccacaaagtg gatgcctgct
2701 gcatcttccc acagtggctt cacagaccca caagagaagc tgatggggag taaaccctgg
2761 agtccgaggc ccaggcagca gccccgccta gtgg gggcc ctgatgctgc caggcctggg
2821 acctcccact gccccctcca ctggaggggt ctcctctgca gctcagggac tggcacactg
2881 gcctccagaa gggcagctcc acagggcagg gcctcattat ttttcactgc cccagacaca
2941 gtgcccaaca ccccgtcgta taccctggat gaacgaatta attacctggc accacctcgt
3001 ctgggctccc tgcgcctgac attcacacag agaggcagag tcccgtgccc attaggtctg
3061 gcatgccccc tcctgcaagg ggctcaaccc cctaccccga cccctccacg tatctttcct
3121 aggcagatca cgttgcaatg gctcaaacaa cattccaccc cagcaggaca gtgaccccag
3181 tcccagctaa ctctgacctg ggagccctca ggcacctgca cttacaggcc ttgctcacag
3241 ctgattgggc acctgaccac acgcccccac aggctctgac cagcagccta tgagggggtt
3301 tggcaccaag ctctgtccaa tcaggtaggc tgggcctgaa ctagccaatc agatcaactc
3361 tgtcttgggc gtttgaactc agggagggag gcccttggga gcaggtgctt gtggacaagg
3421 ctccacaagc gttgagcctt ggaaaggtag acaagcgttg agccactaag cagaggacct
3481 tgggttccca atacaaaaat acctactgct gagagggctg ctgaccattt ggtcaggatt
3541 cctgttgcct ttatatccaa aataaactcc cctttcttga ggttgtctga gtcttgggtc
3601 tatgccttga aaaaagctga attattggac agtctcacct cctgccatag ggtcctgaat
3661 gtttcagacc acaaggggct ccacaccttt gctgtgtgtt ctggggcaac ctactaatcc
3721 tctctgcaag tcggtctcct tatcccccca aatggaaatt gtatttgcct tctccacttt
3781 gggaggctcc cacttcttgg gagggttaca ttttttaagt cttaatcatt tgtgacatat
3841 gtatctatac atccgtatcc tttaatgatc cgtgtgtacc atctttgtga ttatttcctt
3901 aatatttttt ctttaagtca gttcattttc gttgaaatac atttatttaa agaaaaatct
3961 ttgttactct gtaaatgaaa aaacccattt tcgccataaa taaaaggtaa ctgtacaaaa
4021 taagtacaat gcaa
```

An amino acid sequence for the protein (Inositol-trisphosphate 3-kinase A) encoded by the ITPKA gene that is a regulator of T cell exhaustion/dysfunction resistance is available from the UniPROT database at accession no. P23677 or accession number NP_002211, cDNA NM_002220 with gene location at Chr 15: 41.49-41.5 Mb.

```
  (SEQ ID NO: 33)
MTLPGGPTGMARPGGARPCSPGLERAPRRSVGELRLLFEARCAA
VAAAAAAGEPRARGAKRRGGQVPNGLPRAPPAPVIPQLTVTAEEPDVPPTSPGPPERE
RDCLPAAGSSHLQQPRRLSTSSVSSTGSSSLLEDSEDDLLSDSESRSRGNVQLEAGED
VGQKNHWQKIRTMVNLPVISPFKKRYAWVQLAGHTGSFKAAGTSGLILKRCSEPERYC
LARLMADALRGCVPAFHGVVERDGESYLQLQDLLDGFDGPCVLDCKMGVRTYLEEELT
KARERPKLRKDMYKKMLAVDPEAPTEEEHAQRAVTKPRYMQWREGISSSTTLGFRIEG
IKKADGSCSTDFKTTRSREQVLRVFEEFVQGDEEVLRRYLNRLQQIRDTLEVSEFFRR
HEVIGSSLLFVHDHCHRAGVWLIDFGKTTPLPDGQILDHRRPWEEGNREDGYLLGLDN
LIGILASLAER
  (SEQ ID NO: 34)
      1 agtccccggc gcgccgcggg ctggtgggct cagcggcggc gccggcactg ggaaatgacc
    61 ctgcccgggg gcccaacggg catggcgcgg ccggggggcg cgaggccctg cagcccgggg
  121 ctggagcggg ccccgcgccg gagtgtcggg gagctgcgcc tgctcttcga ggcgcgctgt
  181 gcggcggtcg ctgcggccgc cgccgcgggg gagccccggg cccgcggggc caagcggcgt
  241 gggggacagg tccccaacgg gcttccgcgg gctcccccgg ccccggtgat ccctcagctg
  301 accgtgacag ccgaggagcc cgacgtgccc ccgaccagcc ctgggccgcc ggagcgggag
  361 agggactgcc tcccggcagc gggctcttcg cacctgcagc agccgcgccg cctttccacc
  421 tcgtcggtct cctccactgg ctcctcgtcg ctgctcgagg actcggagga cgacctgctg
  481 agcgacagtg agagccggag ccgcggcaac gtgcagctgg aagcgggcga ggacgtgggt
  541 cagaaaaacc actggcagaa gatccggacc atggtcaatc tgccggtcat aagccctttc
  601 aagaagcgct acgcctgggt gcagctggca gggcacactg ggagttttaa ggcggcgggc
  661 accagcgggc tgatcctgaa gcgctgctcg gagccggagc gctactgcct ggcgcggctg
  721 atggctgacg cgctgcgcgg ctgcgtgcct gccttccacg gcgtggtgga gcgcgacggc
  781 gaaagctacc tgcagctgca ggacctgctc gatggcttcg acggaccttg tgtgctcgac
  841 tgcaaaatgg gcgtcaggac ttacctagag gaggagctga ccaaggcccg tgagcggccc
  901 aagctgcgga aggacatgta caagaaaatg ctggcggtgg atcctgaagc tcccacggag
  961 gaggagcacg cgcagcgcgc cgtcaccaag ccgcgctaca tgcagtggcg ggaaggcatc
1021 agctccagca ccaccctcgg cttccgcatc gagggcatca agaaagcgga cggctcctgc
1081 agcaccgact tcaagactac gcgaagccga gagcaggtgc ttcgcgtctt tgaagagttt
1141 gtgcaaggag atgaggaagt gctgaggcgg tatctgaacc gcctgcagca gatccgggac
1201 accctggagg tatccgagtt cttcaggagg cacgaggtga tcggcagctc gctcctcttt
1261 gtgcacgatc actgccatcg cgccggcgtg tggctcatcg acttcggcaa gaccacgccc
1321 ctccccgatg gccagatcct ggaccaccgg cggccctggg aggagggcaa ccgcgaggac
1381 ggctatttgc tggggctgga caatctcatt ggcatcctgg ccagcctggc tgagagatga
1441 ggctggactc ctgtccccgc gggccgctca cctgacatgt ggacctgcag ctttgtcccc
1501 actgtgcatg ccggcttgag actggagccc cgcggtgcag ggcagttcac cgggtcctgc
1561 aggaccaggt gccagccact aagggggggc accgccgatg ccaggggttt tgcccacccg
1621 ggccccagcg ttcccagagc caaatgacac taacttatag aaggggaggg ggcaaagggc
1681 ttcttcctca ggccagctct tctgaggagg ctctgccctc tccagaggtg ccagaccgcg
1741 gattttattt agcaagccca gaccttccgg tctaacgtct cacaccacga cggactcccc
1801 ttcctaataa aactcaaaga caaga
```

An amino acid sequence for the protein (Inositol-trisphosphate 3-kinase B) encoded by the ITPKB gene that is a regulator of T cell exhaustion/dysfunction resistance is available from the UniPROT database at accession no. P27987 or accession number NP_002212, cDNA NM_002221 or NM_001388404 with gene location at Chr 1: 226.63-226.74 Mb.

| (SEQ ID NO: 35) | |
|---|---|
| MAVYCYALNSLVIMNSANEMKSGGGPGPSGSETPPPPRRAVLSP | |
| GSVFSPGRGASFLFPPAESLSPEEPRSPGGWRSGRRRLNSSSGSGSGSSGSSVSSPSW | |
| AGRLRGDRQQVVAAGTLSPPGPEEAKRKLRILQRELQNVQVNQKVGMFEAHIQAQSSA | |
| IQAPRSPRLGRARSPSPCPFRSSSQPPGRVLVQGARSEERRTKSWGEQCPETSGTDSG | |
| RKGGPSLCSSQVKKGMPPLPGRAAPTGSEAQGPSAFVRMEKGIPASPRCGSPTAMEID | |
| KRGSPTPGTRSCLAPSLGLFGASLTMATEVAARVTSTGPHRPQDLALTEPSGRARELE | |
| DLQPPEALVERQGQFLGSETSPAPERGGPRDGEPPGKMGKGYLPCGMPGSGEPEVGKR | |
| PEETTVSVQSAESSDSLSWSRLPRALASVGPEEARSGAPVGGGRWQLSDRVEGGSPTL | |
| GLLGGSPSAQPGTGNVEAGIPSGRMLEPLPCWDAAKDLKEPQCPPGDRVGVQPGNSRV | |
| WQGTMEKAGLAWTRGTGVQSEGTWESQRQDSDALPSPELLPQDPDKPFLRKACSPSNI | |
| PAVIITDMGTQEDGALEETQGSPRGNLPLRKLSSSSASSTGFSSSYEDSEEDISSDPE | |
| RTLDPNSAFLHTLDQQKPRVSKSWRKIKNMVHWSPFVMSFKKKYPWIQLAGHAGSFKA | |
| AANGRILKKHCESEQRCLDRLMVDVLRPFVPAYHGDVVKDGERYNQMDDLLADFDSPC | |
| VMDCKMGIRTYLEEELTKARKKPSLRKDMYQKMIEVDPEAPTEEEKAQRAVTKPRYMQ | |
| WRETISSTATLGFRIEGIKKEDGTVNRDFKKTKTREQVTEAFREFTKGNHNILIAYRD | |
| RLKAIRTTLEVSPFFKCHEVIGSSLLFIHDKKEQAKVWMIDFGKTTPLPEGQTLQHDV | |
| PWQEGNREDGYLSGLNNLVDILTEMSQDAPLA | |
| (SEQ ID NO: 36) | |
| 1 | gggcagtatc tccacggtcg aaaagggcgt gcagggccgg cttggcgtcg ccactgccgg |
| 61 | gatcgccggg cccctgaacc gaagagcttt cccccctctt tcgcactcct cttttttgtc |
| 121 | ttccatagct tgtgagaaaa taatttctga gcatttttac ttttaaagcc atctcgtccc |
| 181 | tacgaggttt gcgcctctgg gcatgtagtc tacacaggac ctgagaatct gagaaactgc |
| 241 | agccgcacgg ttgtttatgg agctttgggc gggggctgag cccgcggtcg tgcccccagc |
| 301 | ccgctgccca ggccatgccg ccccatctgc gcgcggagcc gcggctgccg ggcctccggg |
| 361 | gctgagccgg gagcgccggg aggaggaggc gccggcggcg gagcaggagc gggagccgcg |
| 421 | gcggcgggca gcgcgggacc cagtactatg gctgtgtact gctatgcgct caatagcctg |
| 481 | gtgatcatga atagcgccaa cgagatgaag agcggcggcg gcccggggcc cagtggcagc |
| 541 | gagacgcccc cgcccccgag gagggcagtg ctgagccccg gcagcgtttt cagccccggg |
| 601 | agaggcgcct ctttcctctt ccccccagcc gagtcgctgt cccccgagga gccccggagc |
| 661 | cccgggggct ggcggagcgg ccggcgcagg ctgaatagta gcagcggcag tggcagcggc |
| 721 | agcagcggca gtagcgtgag cagcccaagt tgggctggtc gcctgcgagg ggaccggcag |
| 781 | caggtggtgg cagccggtac cctctccccg ccagggccgg aggaggccaa gaggaagctg |
| 841 | cggatcttgc agcgcgagtt gcagaacgtg caggtgaacc agaaagtggg catgtttgag |
| 901 | gcgcacatcc aggcacagag ctccgccatt caagcgcccc gcagcccgcg tttgggcagg |
| 961 | gctcgctcgc cctccccgtg ccccttccgc agcagcagtc agccccctgg aagggtcctg |
| 1021 | gttcagggcg cccggagcga ggaacggagg acaaagtcct ggggggagca atgtccagag |
| 1081 | acttcaggaa ccgactccgg gaggaaagga gggcccagcc tatgctcctc gcaggtgaag |
| 1141 | aaaggaatgc cacctcttcc cggccgggct gcccctacag gatcagaggc tcagggtcca |
| 1201 | tccgcttttg taaggatgga gaagggtatc cctgccagtc cccgctgtgg ctcacccaca |
| 1261 | gctatggaaa ttgacaaaag gggctctcct accccgggaa ctcggagctg cctagctccc |
| 1321 | tcattggggc tgttcggagc tagcttaacg atggccacgg aagtggcagc gagagttaca |
| 1381 | tccactgggc cacaccgtcc acaggatctt gccctcactg agccgtctgg gagagcccgt |
| 1441 | gagcttgagg acctgcagcc cccagaggcc ctggtggaga ggcaggggca gtttctgggc |
| 1501 | agtgagacaa gcccagcccc agaaaggggc gggccccgcg atggagaacc ccctgggaag |
| 1561 | atggggaaag gatatctgcc ctgtggcatg ccgggctctg gggagcctga agtgggcaaa |
| 1621 | aggccagagg agacgactgt gagcgtgcaa agcgcagagt cctctgattc cctgagctgg |
| 1681 | tccaggctgc ccagggccct ggcctccgta ggccctgagg aggcccgaag tggggccccc |
| 1741 | gtgggcgggg ggcgttggca gctctccgac agagtggagg gagggtcccc aacgctgggc |
| 1801 | ttgcttgggg gcagcccctc agcacagccg gggaccggga atgtggaggc gggaattcct |
| 1861 | tctggcagaa tgctggagcc tttgccctgt tgggacgctg cgaaagatct gaaagaacct |
| 1921 | cagtgccctc ctggggacag ggtgggtgtg cagcctggga actccagggt ttggcagggc |
| 1981 | accatggaga aagccggttt ggcttggacg cgtggcacag gggtgcaatc agaggggact |
| 2041 | tgggaaagcc agcggcagga cagtgatgcc ctcccaagtc cggagctgct accccaagat |
| 2101 | ccggacaagc ctttcctgag gaaggcctgc agccccagca acatacctgc tgtcatcatt |
| 2161 | acagacatgg gcacccagga ggatggggcc ttggaggaga cgcagggaag ccctcggggc |
| 2221 | aacctgcccc tgaggaaact gtcctcttcc tcggcctcct ccacgggctt ctcctcatcc |
| 2281 | tacgaagact cagaggagga catctccagt gaccctgagc gcaccctgga ccccaactca |
| 2341 | gccttcctgc ataccctgga ccagcagaaa cctagagtga gcaaatcatg gaggaagata |
| 2401 | aaaaacatgg tgcactggtc tcccttcgtc atgtccttca agaagaagta cccctggatc |
| 2461 | cagctggcag gacacgcagg gagtttcaag gcagctgcca atggcaggat cctgaagaag |
| 2521 | cactgtgagt cagagcagcg ctgcctggac cggctgatgg tggatgtgct gaggcccttc |
| 2581 | gtacctgcct accatgggga tgtggtgaag gacggggagc gctacaacca gatggacgac |
| 2641 | ctgctggccg acttcgactc gccctgtgtg atggactgca agatgggaat caggacctac |
| 2701 | ctggaggagg agctcacgaa ggcccggaag aagcccagcc tgcggaagga catgtaccag |
| 2761 | aagatgatcg aggtggaccc cgaggccccc accgaggagg aaaaagcaca gcgggctgtg |
| 2821 | accaagccac ggtacatgca gtggcgggag accatcagct ccacggccac cctggggttc |
| 2881 | aggatcgagg gaatcaagaa agaagacggc accgtgaacc gggacttcaa gaagaccaaa |
| 2941 | acgagggagc aggtcaccga ggccttcaga gagttcacta aaggaaacca taacatcctg |
| 3001 | atcgcctatc gggaccggct gaaggccatt cgaaccactc tagaagtttc tcccttcttc |
| 3061 | aagtgccacg aggtcattgg cagctccctc ctcttcatcc acgacaagaa ggaacaggcc |
| 3121 | aaagtgtgga tgatcgactt tcggaaaacc acgcccctgc ctgagggcca gaccctgcag |
| 3181 | catgacgtcc cctggcagga ggggaaccgg gaggatggct acctctcggg gctcaataac |
| 3241 | ctcgtcgaca tcctgaccga gatgtcccag gatgccccac tcgcctgagc tgcccacgcc |
| 3301 | ctccctggcc cccgcctggg cctcctttcc tcctcctgtg cttcctttct cgttcctaac |
| 3361 | ttttccttca cttacacctg actgaccctc ctgaactgca ctacaagaca ctttgtagaa |
| 3421 | gaggagatga gagtttctag tcattttcct aacttcaggg cttggaggtg gtgtttgcac |
| 3481 | tgctttttgt agagagggtc acctactaga agagaaatgc ccagtcttag aggtgggtca |
| 3541 | ggtgtagagc tggagggggt ccctggctgc tgaggggacc ctaccagatg agccctgcct |
| 3601 | ctgggagccc cctaggaagc accagcctgg acctaccacc tgcggaggcc tgctgccccc |
| 3661 | tggcggccag tgctgttaga gtgctgccaa gcacagcctt atttctgccg gggcctcccc |
| 3721 | accggagagc ccagggggcc ggccgggttc ctggtccctg gctgggagca gggctttctg |
| 3781 | gtagttgggg cacaaaacca tcggggaacc acatgttgac tgtgagcaaa gtgtcttccg |
| 3841 | attagcagcc tcagggatgc cctggtggcc tctccagggc tgctcaggca aggcccccca |
| 3901 | cccatctggt atggaaacct gccggctcca ggccagaccc aggagccaag agaaggctga |
| 3961 | agccagcttc gctgtgttct ctgatctagg ccttcccaga ggaggcgagc agaagctgtg |
| 4021 | ccacttggaa ttgcaaccca tgagttcaga aggcacactc tgccatgctg agctccaagg |
| 4081 | gtgctaccag gggaagatgg gatctataga gtctctgggc cctggcccca gggaggagca |
| 4141 | catttttctt gaccctcacc tacctggtgc tagttggtca accctgcctg catacatggg |
| 4201 | ctcctgtcat ggggcccaga gtcccttgca gatatagaaa taggggagga gctcaggtct |
| 4261 | gcgccaggca ggaagaaggc aggcttctgg cttccagagg tgccgcggtg gcctcctggc |
| 4321 | atcatttgtt attgcctctg aaacaagcct tactgcctgg agggcttaga ttcctgcttc |
| 4381 | tccaatgtag tgtgggtatc ttgtagggta tgtggtggat gccagggcgt gctccaggca |
| 4441 | cctcttcctg aagtctctgc atttggagat tcgtggagaa cctatttaag cccaatttta |
| 4501 | actgaaagcc agtgagtctg atatggaagg gaatgtaaaa tttgcctgac ttcttaagaa |
| 4561 | caaaaccccc agctctgtgc cccatgctcc ttggggcttg ccacccactc ctttgctgtc |
| 4621 | agaggtacag gagctgggag agtccaggag ctagggacac agagggagac tatggaccaa |
| 4681 | ggtgtgtgtg tctggaggaa ccactgccca ccccaccacc ccggggtctc tggggaactg |
| 4741 | tcaacctgcc cacgggacat gtacatttcc ccttatgtgc tggaagtgtg agtgacactt |
| 4801 | gctgggggtg gagggtggga cacatgagga tgtataagta cagattttaa aaaaggaaat |
| 4861 | caacttacac ttcctggctc ttgtttaaaa cagtggtgag ctcctgtgtg ggccgacttg |
| 4921 | ctaaaggtca cacacgcgcc cggtggagca cgagagacct cgtggcagca tgtgatctgg |
| 4981 | aaggcaggca ggacgggggc gttggggagc caaagtcaac tctgggcctc tggagctata |
| 5041 | gtgacttttg ggctagaagg gaccctggtg gtctgtgctt cagccatttg cagggcaggg |
| 5101 | gcatcattaa ttcagacgta aagattctat gaatatggac tggccaaaag ttatccttac |
| 5161 | tccatctgtg aaagaagttt gctaaagcaa atcatgatat gaacaaaaat tacaggggac |
| 5221 | ctgtttaaga gaacaaaatg ttccaagcac tttaggcaga caccagctgt ttgcaaacaa |
| 5281 | tgtgctaata tgcaaatgat gtgcttatta aaggaggccc atggggcctc ttattggcaa |
| 5341 | tacttggctg tgggttacat taaatatgtg aacatagtat gaagtagcat cattttaggg |
| 5401 | ttattctgtt acttagggtt tttgttttct gttttttttt tctctttttt tgtatttacc |
| 5461 | gtgctagttc tcttctacac ctactctgtc tctcaagcca ttttgccact cgcttccctg |
| 5521 | ccatctggcc cttccctttg tctcagtggg atagatggat tgtgaaatgg aatctcccag |
| 5581 | aacccctgcc ctggcagcct ggaagaccgt gcctgcccag ccctcgtcac cacagggact |
| 5641 | ccttgggtcc tggcagtgca tgtgccagca ggcaggacaa actctgtgta cctgtgccca |
| 5701 | ggtgaatggg cgcagggtcc tcttgccctg tcctgcgggg ggccccacga gttcctggca |
| 5761 | ttcagcactg cttagcattc tcggaaggtt tcttcaactg cttgcttttc ccaggcttgc |
| 5821 | ctttagtgtc atgtaagaca tttttaagtt atatttattt tgttgggttt taaaattgca |
| 5881 | cagaacacta agaccgaaag gctggactct tgtttctcct tgaaagcttt gcctttgttt |
| 5941 | tgaacttcct ttcccacttg gtagaaagag cccagaagca gccctggccc tgtaagatgg |
| 6001 | actctttcat ccttcagttg tatttagctt tgagcttctc tgcatctgtc caccccatgt |
| 6061 | gtatataacc cagcccctgg ctctggggtg gtcacctcgt cagtgccttt tgttctggag |
| 6121 | gagaggaccc cccccgcctg ccgagaggct ctcttcctgt tctgcacccc tctccccatg |
| 6181 | ggaccttgga gaaaactgaa ctgttacaaa cccctgcaca gtgcctgtca aacagatgca |
| 6241 | aaccttcctg aataaagcct tggagacca |

An amino acid sequence for the protein (Interleukin 10 (IL-10)), also known as human cytokine synthesis inhibitory factor (CSIF)) encoded by the IL10 gene that is a regulator of T cell exhaustion/dysfunction resistance is available from the UniPROT database at accession no. P22301 or accession number P22301. cDNA NM_000572 with gene location at Chr 1: 206.77-206.77 Mb.

| (SEQ ID NO: 37) | |
|---|---|
| MHSSALLCCLVLLTGVRASPGQGTQSENSCTHFPGNLPNMLRDL | |
| RDAFSRVKTFFQMKDQLDNLLLKESLLEDFKGYLGCQALSEMIQFYLEEVMPQAENQD | |
| PDIKAHVNSLGENLKTLRLRLRRCHRFLPCENKSKAVEQVKNAFNKLQEKGIYKAMSE | |
| FDIFINYIEAYMTMKIRN | |
| (SEQ ID NO: 38) | |
| 1 | acacatcagg ggcttgctct tgcaaaacca aaccacaaga cagacttgca aaagaaggca |
| 61 | tgcacagctc agcactgctc tgttgcctgg tcctcctgac tggggtgagg gccagcccag |
| 121 | gccagggcac ccagtctgag aacagctgca cccacttccc aggcaacctg cctaacatgc |
| 181 | ttcgagatct ccgagatgcc ttcagcagag tgaagacttt ctttcaaatg aaggatcagc |
| 241 | tggacaactt gttgttaaag gagtccttgc tggaggactt taagggttac ctgggttgcc |
| 301 | aagccttgtc tgagatgatc cagttttacc tggaggaggt gatgccccaa gctgagaacc |
| 361 | aagacccaga catcaaggcg catgtgaact ccctggggga gaacctgaag accctcaggc |
| 421 | tgaggctacg gcgctgtcat cgatttcttc cctgtgaaaa caagagcaag gccgtggagc |
| 481 | aggtgaagaa tgcctttaat aagctccaag agaaaggcat ctacaaagcc atgagtgagt |
| 541 | ttgacatctt catcaactac atagaagcct acatgacaat gaagatacga aactgagaca |
| 601 | tcagggtggc gactctatag actctaggac ataaattaga ggtctccaaa atcggatctg |
| 661 | gggctctggg atagctgacc cagccccttg agaaacctta ttgtacctct cttatagaat |
| 721 | atttattacc tctgatacct caacccccat ttctatttat ttactgagct tctctgtgaa |
| 781 | cgatttagaa agaagcccaa tattataatt tttttcaata tttattattt tcacctgttt |
| 841 | ttaagctgtt tccatagggt gacacactat ggtatttgag tgttttaaga taaattataa |
| 901 | gttacataag ggaggaaaaa aaatgttctt tggggagcca acagaagctt ccattccaag |
| 961 | cctgaccacg ctttctagct gttgagctgt tttccctgac ctccctctaa tttatcttgt |
| 1021 | ctctgggctt ggggcttcct aactgctaca aatactctta ggaagagaaa ccagggagcc |
| 1081 | cctttgatga ttaattcacc ttccagtgtc tcggagggat tcccctaacc tcattcccca |
| 1141 | accacttcat tcttgaaagc tgtggccagc ttgttattta taacaaccta aatttggttc |
| 1201 | taggccgggc gcggtggctc acgcctgtaa tcccagcact ttgggaggct gaggcgggtg |
| 1261 | gatcacttga ggtcaggagt tcctaaccag cctggtcaac atggtgaaac cccgtctcta |
| 1321 | ctaaaaatac aaaaattagc cgggcatggt ggcgcgcacc tgtaatccca gctacttggg |
| 1381 | aggctgaggc aagagaattg cttgaaccca ggagatggaa gttgcagtga gctgatatca |
| 1441 | tgcccctgta ctccagcctg ggtgacagag caagactctg tctcaaaaaa taaaaataaa |
| 1501 | aataaatttg gttctaatag aactcagttt taactagaat ttattcaatt cctctgggaa |
| 1561 | tgttacattg tttgtctgtc ttcatagcag attttaattt tgaataaata aatgtatctt |
| 1621 | attcacatca |

An amino acid sequence for the protein (Interleukin 9 receptor (IL9R)) also known as CD129), also known as human cytokine synthesis inhibitory factor (CSIF)) encoded by the IL9R gene that is a regulator of T cell exhaustion/dysfunction resistance is available from the UniPROT database at accession no. Q01113 or accession number NP_002177 or NP_789743, cDNA NM_002186 or NM_176786 with gene location at Chr X: 156-156.01 Mb.

| (SEQ ID NO: 39) | |
|---|---|
| MGLGRCIWEGWTLESEALRRDMGTWLLACICICTCVCLGVSVTG | |
| EGQGPRSRTFTCLTNNILRIDCHWSAPELGQGSSPWLLFTSNQAPGGTHKCILRGSEC | |
| TVVLPPEAVLVPSDNFTITFHHCMSGREQVSLVDPEYLPRRHVKLDPPSDLQSNISSG | |
| HCILTWSISPALEPMTTLLSYELAFKKQEEAWEQAQHRDHIVGVTWLILEAFELDPGF | |
| IHEARLRVQMATLEDDVVEEERYTGQWSEWSQPVCFQAPQRQGPLIPPWGWPGNTLVA | |
| VSIFLLLTGPTYLLFKLSPRVKRIFYQNVPSPAMFFQPLYSVHNGNFQTWMGAHGAGV | |
| LLSQDCAGTPQGALEPCVQEATALLTCGPARPWKSVALEEEQEGPGTRLPGNLSSEDV | |
| LPAGCTEWRVQTLAYLPQEDWAPTSLTRPAPPDSEGSRSSSSSSSSNNNNYCALGCYG | |
| GWHLSALPGNTQSSGPIPALACGLSCDHQGLETQQGVAWVLAGHCQRPGLHEDLQGML | |
| LPSVLSKARSWTF | |
| (SEQ ID NO: 40) | |
| 1 | gctgtgcacc cagagatagt tgggtgacaa atcacctcca ggttggggat gcctcagact |
| 61 | tgtgatggga ctgggcagat gcatctggga aggctggacc ttggagagtg aggccctgag |
| 121 | gcgagacatg ggcacctggc tcctggcctg catctgcatc tgcacctgtg tctgcttggg |
| 181 | agtctctgtc acaggggaag gacaagggcc aaggtctaga accttcacct gcctcaccaa |
| 241 | caacattctc aggatcgatt gccactggtc tgccccagag ctgggacagg gctccagccc |
| 301 | ctggctcctc ttcaccagca accaggctcc tggcggcaca cataagtgca tcttgcgggg |
| 361 | cagtgagtgc accgtcgtgc tgccacctga ggcagtgctc gtgccatctg acaatttcac |
| 421 | catcactttc caccactgca tgtctgggag ggagcaggtc agcctggtgg acccggagta |
| 481 | cctgccccgg agacacgtta agctggaccc gccctctgac ttgcagagca acatcagttc |
| 541 | tggccactgc atcctgacct ggagcatcag tcctgccttg gagccaatga ccacacttct |
| 601 | cagctatgag ctggccttca agaagcagga agaggcctgg gagcaggccc agcacaggga |
| 661 | tcacattgtc ggggtgacct ggcttatact tgaagccttt gagctggacc ctggctttat |
| 721 | ccatgaggcc aggctgcgtg tccagatggc cacactggag gatgatgtgg tagaggagga |
| 781 | gcgttataca ggccagtgga gtgagtggag ccagcctgtg tgcttccagg ctccccagag |
| 841 | acaaggccct ctgatcccac cctgggggtg gccaggcaac acccttgttg ctgtgtccat |
| 901 | ctttctcctg ctgactggcc cgacctacct cctgctcaag ctgtcgccca gggtgaagag |
| 961 | aatcttctac cagaacgtgc cctctccagc gatgttcttc cagcccctct acagtgtaca |
| 1021 | caatgggaac ttccagactt ggatgggggc ccacggggcc ggtgtgCtgt tgagccagga |
| 1081 | ctgtgctggc accccacagg gagccttgga gccctgcgtc caggaggcca ctgcactgct |
| 1141 | cacttgtggc ccagcgcgtc cttggaaatc tgtggccctg gaggaggaac aggagggccc |
| 1201 | tgggaccagg ctcccgggga acctgagctc agaggatgtg ctgccagcag ggtgtacgga |
| 1261 | gtggagggta cagacgcttg cctatctgcc acaggaggac tgggccccca cgtccctgac |
| 1321 | taggccggct cccccagact cagagggcag caggagcagc agcagcagca gcagcagcaa |
| 1381 | caacaacaac tactgtgcct tgggctgcta tgggggatgg cacctctcag ccctcccagg |
| 1441 | aaacacacag agctctgggc ccatcccagc cctggcctgt ggcctttctt gtgaccatca |
| 1501 | gggcctggag acccagcaag gagttgcctg ggtgctggct ggtcactgcc agaggcctgg |
| 1561 | gctgcatgag gacctccagg gcatgttgct cccttctgtc ctcagcaagg ctcggtcctg |
| 1621 | gacattctag gtccctgact cgccagatgc atcatgtcca ttttgggaaa atggactgaa |
| 1681 | gtttctggag cccttgtctg agactgaacc tcctgagaag gggcccctag cagcggtcag |
| 1741 | aggtcctgtc tggatggagg ctggaggctc ccccctcaac ccctctgctc agtgcctgtg |
| 1801 | gggagcagcc tctaccctca gcatcctggc cacaagttct tccttccatt gtcccttttc |
| 1861 | tttatccctg acctctctga gaagtggggt gtggtctctc agctgttctg ccctcatacc |
| 1921 | cttaaagggc cagcctgggc ccagtggaca caggtaaggc accatgacca cctggtgtga |
| 1981 | cctctctgtg ccttactgag gcacctttct agagattaaa aggggcttga tggctgtt |

An amino acid sequence for the protein (LIFR also known as CD118 (Cluster of Differentiation 118)) also known as CD129), also known as human cytokine synthesis inhibitory factor (CSIF)) encoded by the LIFR gene that is a regulator of T cell exhaustion/dysfunction resistance is available from the UniPROT database at accession no. P42702 or accession number NP 001121143, NP_002301, NP_001351226 or NP_001351227, cDNA NM_001127671, NM_002310, NM_001364297 or NM_001364298 with gene location at Chr 5: 38.47-38.61 Mb.

| (SEQ ID NO: 41) | |
|---|---|
| MMDIYVCLKRPSWMVDNKRMRTASNFQWLLSTFILLYLMNQVNS | |
| QKKGAPHDLKCVTNNLQVWNCSWKAPSGTGRGTDYEVCIENRSRSCYQLEKTSIKIPA | |
| LSHGDYEITINSLHDFGSSTSKFTLNEQNVSLIPDTPEILNLSADFSTSTLYLKWNDR | |
| GSVFPHRSNVIWEIKVLRKESMELVKLVTHNTTLNGKDTLHHWSWASDMPLECAIHFV | |
| EIRCYIDNLHFSGLEEWSDWSPVKNISWIPDSQTKVFPQDKVILVGSDITFCCVSQEK | |
| VLSALIGHTNCPLIHLDGENVAIKIRNISVSASSGTNVVFTTEDNIFGTVIFAGYPPD | |
| TPQQLNCETHDLKEIICSWNPGRVTALVGPRATSYTLVESFSGKYVRLKRAEAPTNES | |
| YQLLFQMLPNQEIYNFTLNAHNPLGRSQSTILVNITEKVYPHTPTSFKVKDINSTAVK | |
| LSWHLPGNFAKINFLCEIEIKKSNSVQEQRNVTIKGVENSSYLVALDKLNPYTLYTFR | |
| IRCSTETFWKWSKWSNKKQHLTTEASPSKGPDTWREWSSDGKNLIIYWKPLPINEANG | |
| KILSYNVSCSSDEETQSLSEIPDPQHKAEIRLDKNDYIISVVAKNSVGSSPPSKIASM | |
| EIPNDDLKIEQVVGMGKGILLTWHYDPNMTCDYVIKWCNSSRSEPCLMDWRKVPSNST | |
| ETVIESDEFRPGIRYNFFLYGCRNQGYQLLRSMIGYIEELAPIVAPNFTVEDTSADSI | |
| LVKWEDIPVEELRGELRGYLFYFGKGERDTSKMRVLESGRSDIKVKNITDISQKTLRI | |
| ADLQGKTSYHLVLRAYTDGGVGPEKSMYVVTKENSVGLIIAILIPVAVAVIVGVVTSI | |
| LCYRKREWIKETFYPDIPNPENCKALQFQKSVCEGSSALKTLEMNPCTPNNVEVLETR | |
| SAFPKIEDTEIISPVAERPEDRSDAEPENHVVVSYCPPIIEEEIPNPAADEAGGTAQV | |
| IYIDVQSMYQPQAKPEEEQENDPVGGAGYKPQMHLPINSTVEDIAAEEDLDKTAGYRP | |
| QANVNTWNLVSPDSPRSIDSNSEIVSFGSPCSINSRQFLIPPKDEDSPKSNGGGWSFT | |
| NFFQNKPND | |
| (SEQ ID NO: 42) | |
| 1 | agtcccggct ggagcgactc ggccgtggcg gtggccgtta ccgttgcctc tgggaggctc |
| 61 | tggcggcggc ggcggcccgc ggggttcgcg gagcgcagcg ggccgccctt cgcccgtggc |
| 121 | cgcctcaagc agcctcgcgc cgacacaggg gaggcgagac ggcggctgcg gaggcggcgg |
| 181 | cgcattgtgc ccggccccct ccttggcggc tcctgccccc gggcggctgt gggatctggg |
| 241 | cagccccggg gcggcggccg aggctacaga gccggcggca ggggatggca agatagcggc |
| 301 | gcgggagcgt accgactgac tgcattgcac agatgatgga tatttacgta tgtttgaaac |
| 361 | gaccatcctg gatggtggac aataaaagaa tgaggactgc ttcaaatttc cagtggctgt |
| 421 | tatcaacatt tattcttcta tatctaatga atcaagtaaa tagccagaaa aagggggctc |
| 481 | ctcatgattt gaagtgtgta actaacaatt tgcaagtgtg gaactgttct tggaaagcac |
| 541 | cctctggaac aggccgtggt actgattatg aagtttgcat tgaaaacagg tcccgttctt |
| 601 | gttatcagtt ggagaaaacc agtattaaaa ttccagctct ttcacatggt gattatgaaa |
| 661 | taacaataaa ttctctacat gattttggaa gttctacaag taaattcaca ctaaatgaac |
| 721 | aaaacgtttc cttaattcca gatactccag agatcttgaa tttgtctgct gatttctcaa |
| 781 | cctctacatt atacctaaag tggaacgaca ggggttcagt ttttccacac cgctcaaatg |
| 841 | ttatctggga aattaaagt= ctacgtaaag agagratgga gctcgtaaaa ttagtgaccc |
| 901 | acaacacaac tctgaatggc aaagatacac ttcatcactg gagttgggcc tcagatatgc |
| 961 | ccttggaatg tgccattcat tttgtggaaa ttagatgcta cattgacaat cttcattttt |
| 1021 | ctggtctcga agagtggagt gactggagcc ctgtgaagaa catttcttgg atacctgatt |
| 1081 | ctcagactaa ggtttttcct caagataaag tgatacttgt aggctcagac ataacatttt |
| 1141 | gttgtgtgag tcaagaaaaa gtgttatcag cactgattgg ccatacaaac tgccccttga |
| 1201 | tccatcttga tggggaaaat gttgcaatca agattcgtaa tatttctgtt tctgcaagta |
| 1261 | gtggaacaaa tgtagttttt acaaccgaag ataacatatt tggaaccgtt atttttgctg |
| 1321 | gatatccacc agatactcct caacaactga attgtgagac acatgattta aaagaaatta |
| 1381 | tatgtagttg gaatccagga agggtgacag cgttggtggg cccacgtgct acaagctaca |
| 1441 | ctttagttga aagtttttca ggaaaatatg ttagacttaa aagagctgaa gcacctacaa |
| 1501 | acgaaagcta tcaattatta tttcaaatgc ttccaaatca agaaatatat aattttactt |
| 1561 | tgaatgctca caatccgctg ggtcgatcac aatcaacaat tttagttaat ataactgaaa |
| 1621 | aagtttatcc ccatactcct acttcattca aagtgaagga tattaattca acagctgtta |
| 1681 | aactttcttg gcatttacca ggcaactttg caaagattaa ttttttatgt gaaattgaaa |
| 1741 | ttaagaaatc taattcagta caagagcagc ggaatgtcac aatcaaagga gtagaaaatt |
| 1801 | caagttatct tgttgctctg gacaagttaa atccatacac tctatatact tttcggattc |
| 1861 | gttgttctac tgaaactttc tggaaatgga gcaaatggag caataaaaaa caacatttaa |
| 1921 | caacagaagc cagtccttca aaggggcctg atacttggag agagtggagt tctgatggaa |
| 1981 | aaaatttaat aatctattgg aagcctttac ccattaatga agctaatgga aaaatacttt |
| 2041 | cctacaatgt atcgtgttca tcagatgagg aaacacagtc cctttctgaa atccctgatc |
| 2101 | ctcagcacaa agcagagata cgacttgata agaatgacta catcatcagc gtagtggcta |
| 2161 | aaaattctgt gggctcatca ccaccttcca aaatagcgag tatggaaatt ccaaatgatg |
| 2221 | atctcaaaat agaacaagtt gttgggatgg gaaaggggat tctcctcacc tggcattacg |
| 2281 | accccaacat gacttgcgac tacgtcatta agtggtgtaa ctcgtctcgg tcggaaccat |
| 2341 | gccttatgga ctggagaaaa gttccctcaa acagcactga aactgtaata gaatctgatg |
| 2401 | agtttcgacc aggtataaga tataattttt tcctgtatgg atgcagaaat caaggatatc |
| 2461 | aattattacg ctccatgatt ggatatatag aagaattggc tcccattgtt gcaccaaatt |
| 2521 | ttactgttga ggatacttct gcagattcga tattagtaaa atgggaagac attcctgtgg |
| 2581 | aagaacttag aggcttttta agaggatatt tgttttactt tggaaaagga gaaagagaca |
| 2641 | catctaagat gagggtttta gaatcaggtc gttctgacat aaaagttaag aatattactg |
| 2701 | acatatccca gaagacactg agaattgctg atcttcaagg taaaacaagt taccacctgg |
| 2761 | tcttgcgagc ctatacagat ggtggagtgg gcccggagaa gagtatgtat gtggtgacaa |
| 2821 | aggaaaattc tgtgggatta attattgcca ttctcatccc agtggcagtg gctgtcattg |
| 2881 | ttggagtggt gacaagtatc ctttgctatc ggaaacgaga atggattaaa gaaaccttct |
| 2941 | accctgatat tccaaatcca gaaaactgta aagcattaca gtttcaaaag agtgtctgtg |
| 3001 | agggaagcag tgctcttaaa acattggaaa tgaatccttg taccccaaat aatgttgagg |
| 3061 | ttctggaaac tcgatcagca tttcctaaaa tagaagatac agaaataatt tccccagtag |
| 3121 | ctgagcgtcc tgaagatcgc tctgatgcag agcctgaaaa ccatgtggtt gtgtcctatt |
| 3181 | gtccacccat cattgaggaa gaaataccaa acccagccgc agatgaagct ggagggactg |
| 3241 | cacaggttat ttacattgat gttcagtcga tgtatcagcc tcaagcaaaa ccagaagaag |
| 3301 | aacaagaaaa tgaccctgta ggaggggcag gctataagcc acagatgcac ctccccatta |
| 3361 | attctactgt ggaagatata gctgcagaag aggacttaga taaaactgcg ggttacagac |
| 3421 | ctcaggccaa tgtaaataca tggaatttag tgtcuccaga ctctcctaga tccatagaca |
| 3481 | gcaacagtga gattgtctca tttggaagtc catgctccat taattcccga caatttttga |
| 3541 | ttcctcctaa agatgaagac tctcctaaat ctaatggagg agggtggtcc tttacaaact |
| 3601 | tttttcagaa caaaccaaac gattaacagt gtcaccgtgt cacttcagtc agccatctca |
| 3661 | ataagctctt actgctagtg ttgctacatc agcactgggc attcttggag ggatcctgtg |
| 3721 | aagtattgtt aggaggtgaa cttcactaca tgttaagtta cactgaaagt gttcatgtgc |
| 3781 | ttttaatgta gtctaaaagc caaagtatag tgactcagaa tcctcaatcc acaaaactca |
| 3841 | agattgggag ctctttgtga tcaagccaaa gaattctcat gtactctacc ttcaagaagc |
| 3901 | atttcaaggc taatacctac ttgtacgtac atgtaaaaca aatcccgccg caactgtttt |
| 3961 | ctgttctgtt gtttgtggtt ttctcatgtg tatacttggt ggaattgtaa gtggatttgc |
| 4021 | aggccaggga gaaaatgtcc aagtaacagg tgaagtttat ttgcctgacg tttactcctt |
| 4081 | tctagatgaa aaccaagcac agattttaaa acttctaaga ttattctcct ctatccacag |
| 4141 | cattcacaaa aattaatata atttttaatg tagtgacagc gatttagtgt tttgtttgat |
| 4201 | aaagtatgct tatttctgtg cctactgtat aatggttatc aaacagttgt ctcaggggta |
| 4261 | caaactttga aaacaagtgt gacactgacc agcccaaatc ataatcatgt tttcttgctg |
| 4321 | tgataggttt tccaagcctt ttcattattt tttagctttt atgcttgctt ccattatttc |
| 4381 | agttggttgc cctaatattt aaaatttaca cttctaagac tagagaccca cattttttaa |
| 4441 | aaatcatttt attttgtgat acagtgacag ctttatatga gcaaattcaa tattattcat |
| 4501 | aagcatgtaa ttccagtgac ttactatgtg agatgactac taagcaatat ctagcagcgt |
| 4561 | tagctgtcca tatagttctg attggatttc gttcctcctg aggagaccat gccgttgagc |
| 4621 | ttggctaccc aggcagtggt gatctttgac accttctggt ggatgttcct cccactcatg |
| 4681 | agtcttttca tcatgccaca ttatctgatc cagtcctcac atttttaaat ataaaactaa |
| 4741 | agagagaatg cttcttacag gaacagttac ccaagggctg tttcttagta actgtcataa |
| 4801 | actgatctga atccatgggc atacctgtgt tgcaggtgca gcaattgctt ggtgagctgt |
| 4861 | gcagaattga ttgccttcag cacagcatcc tctgcccacc cttgtttctc ataagcgatg |
| 4921 | tctggagtga ttgtggttct tggaaaagca gaaggaaaaa ctaaaaagtg tatcttgtat |
| 4981 | tttccctgcc ctcaggttgc ctatgtattt taccttttca tatttaaggc aaaagtactt |
| 5041 | gaaaatttta agtgtccgaa taagatatgt cttttttgtt tgtttttttt ggttgtttgt |
| 5101 | ttgtttttta tcatctgaga ttctgtaatg tattcgcaaa taatggatca attaattttt |
| 5161 | tttgaagctc atattgtatc tttttaaaaa ccatgttgtg gaaaaaagcc agagtgacaa |
| 5221 | gtgacaaaat ctatttaggt tctctgtgta tgaatcctga ttttaactgc taggattcag |
| 5281 | ctaaatttct gagctttatg atctgtggaa atttggaatg aaatgcaatt cattttgtac |
| 5341 | atacatagta tattaaaact atataatagt tcatagaaat gttcagtaat gaaaaatata |
| 5401 | tccaatcaga gccatcccta aagagtgttc tcttgtcttc ctttgtatcc tctttggctc |
| 5461 | cttcccttag cttccacccc agacatcaga cctggggctg ccttctctcc ctgccaaagc |
| 5521 | ttgctgttag cattgtctgc ttgccctcaa ctcctgcctt gttccctgtt gaaatgtatc |
| 5581 | aatcccgtac tttatctctc caggacaagg ataagtgtgg ctacagtaaa agtttattgc |
| 5641 | tctaaacggc gctaacctga gataatgaga aaaggccaga agagattgtt tgatctctgt |
| 5701 | ctgtcaatat atatgcattt caactgtgac catchggatc cagggccctt tctaacctat |
| 5761 | ataacgactc atttgtttgg ttaatgatga atatcactgg ggctcttaag tcctttcgtt |
| 5821 | ctatccaaga cttcattgtt tttttctgta tgaaaaaagt gcagaatatc actcaccttt |
| 5881 | cccctacttt tggatcccct acacgaggat cactagctta atgagaagca gagtaacgct |
| 5941 | attaagtctg ggcaagctcc ttataagttg ctgtgtgctt aaaagaatgt gatttatgtc |
| 6001 | tggggccaaa gcagttttcc ttcttttccc tcattgaatt caagacagct agacccttgt |
| 6061 | gtagatccca ggatattacc tctactcccc ccaacctgtg tgtgtaggga gagtaacaac |
| 6121 | acgggaagag gggagggaag ttggaaggag attggtcaca tgttcagttg tctgagatac |
| 6181 | tggtaaatgt gcgctgtttg tcaatgaagg ttgcatgcct gcatttacaa ggtggtctca |
| 6241 | tactgattgt gctctgtcca gttagattcg tggcttctcc cttggaagct acaattaagg |
| 6301 | actagttaga agcagaggct agatgtgttc tctccagccc ttactaactg ggaaaataat |
| 6361 | tgagaaatcc ctcttttcct cagtgaaagc ccagtcattg gttagaaaaa tctaattgtc |
| 6421 | aaactagcat tatgttccat catatctgtg ggatagtaat tcatgtaaga cacattttta |
| 6481 | cctctcttac aagtcaattc acttacaagc cagtttcaaa tcaagcttcc cttgcgagga |
| 6541 | atggagatca ttatggggct tattatgact acatttcttt tatgagtgac cattttaaat |
| 6601 | aattaatatt ttatttaata ttaaattatt aaagacttag ctagactttt aacattagat |
| 6661 | gaaccttgtg aatcatatat tgcatagatt agcacatgta tgcaatctag gtcatcaatt |
| 6721 | tgttgaattt atacagccct agatttgaga ttgtggccct gagtatctgg atagttgagt |
| 6781 | tgtgtgttta tgtgcttata tgatagaggt gctttgttcc tgcagcttag aaacagaagc |
| 6841 | agccattcac tgagaagctt aagagaggaa gtatcaacct cattttccaa accagcatta |
| 6901 | ctttcaccgc tcgtggatcz gaaagattac atttagaaca tttagtacta ttacactctc |
| 6961 | agaaactgtg gtaaaaagtc acattttcaa aacttcatgc atattgtatt cttgttggaa |
| 7021 | ataagtccta tagtttctta attgtcttca tgccgtccat atttaacaaa cattgcaagt |
| 7081 | cctttttata tttgggtaat tatttgtaat ttag gaagg agactagggg atgttttctt |
| 7141 | ccaaagggaa tttaaaatca attttatggt attttgaaag taaaatactc cttaacgtgt |
| 7201 | caatattttt aaaatgttat ctcgaagttt tctattgctc aggtatataa tctaataaaa |
| 7261 | ttttttgttt gctctaaggt tatttttgct attctctatt ttgaattatg ttaaaatttg |
| 7321 | tgtgcatttt atgaaatgcc ttttttctta ttttgaaagt gtaaaattgg gcatctcaga |
| 7381 | gtaaaatgtg ggattattag ctacaaagac taggcacatc gtgaaagctc agtaactaat |
| 7441 | tttcttgact gagtcctatc ccagagtgtc acagattcaa cagaattttc ctgaagggtg |
| 7501 | atacacacca aaatctagct tgtgtggatc tgagactatt gactcaaaga ttcaaatcta |
| 7561 | cttcggaagt gactccacgg tttgagtgaa aactgaggaa ggactcatgc ctgtatatca |
| 7621 | ctagatatct tatttctcca taattttaag tgttcataat ttcttcttct ttgatgggtg |
| 7681 | tgcaactggg gcatgtttgt accagccagc atgtgttctg cccaacagtg tgtccgtgtg |
| 7741 | tgaggttaac tgattcagaa ctcattctct tgataaacta tttcttgaaa gcatttattt |
| 7801 | taagtcttta tttcctgctg aaatgaagtt tcatgctgtc atgcccatct tatttattct |
| 7861 | agaagaaaat tttcatagaa gaaaatgatt tatggccaaa gttacagaac attcatcttt |
| 7921 | tagacttagc cttagaatat cattggaaat tactcggctt gaatttggtt cgtttttatc |
| 7981 | tttatggatt atgtaccagg gctattaata atacttaaac cccttatttt gctatggtgg |
| 8041 | ttagattttt ttattccttc tcaattagtg ctattaatta ttacatggtc gtattcatgc |
| 8101 | ttattacaga tgaactatt: ttagtgccag tgtaaaggat taagaataac attcaattag |
| 8161 | ttggctattt caggctttat tgtacatggt aatgtttcag ggagtgtgat ttggtctaga |
| 8221 | atatatccag atccttgtaa ttatgatatt gggttatgac aggattctat tatttttagc |
| 8281 | tatgaatggt gcttattttt ccctatatta ggaatgttaa tatttggatt atcagagtcc |
| 8341 | atgttataaa cagaatttac ctatgcaaga caaattctaa ctgtgcaaca ttttcatgag |
| 8401 | aattaaaagt tgtgtgttaa ggggaaaacc ctctgaatgt ttttatgtgt aagtgcctaa |
| 8461 | agaaccgtag aaacagtaca tcaaaagctc ttataaaaga aaaactgatt gaacattagc |
| 8521 | cagaaacaaa atatgattta catttttttt tttggttgct gggactgaat accctaaaaa |
| 8581 | ctaagagatg tgagtgggga aaatgtctaa ttgataagaa agttgattta tattatattt |
| 8641 | acatgcatct tctaagattg cctttacagt agccaatcag aactttaaaa aaaaaaaagc |
| 8701 | taacaacata gatcctatgt gttgccctta agactaaaat actttgcaca cgtgattgaa |
| 8761 | tggctcactt aagttgttga atttccttat acatttaaag tgtacttccg ttaagaatta |
| 8821 | ttccatttta aatgggtaac tttcagcttc cgaactattt tcctaaacat tccagttgtg |
| 8881 | tattataaaa gatcaaaata tctctaggtg gtagtacaat tttcatttat aacgtggaaa |
| 8941 | agttaactgt taggtttaac attagtatta gtgtttttgc aattgctcat agaaaagaac |
| 9001 | actgaataat ggttttcata caatttttgt aaaacattct tctcccagtt tgattccaaa |
| 9061 | tgcttttccc caaattgtcz aatatatcat aagctagtgt ttccttgtat ttaatttttc |
| 9121 | cttccaacac aatatttagg aaaaatatga ataacattgt gagtagcggg agacaacacg |
| 9181 | aaaaagaaac ctttcattgt tgtggagggg taggtgttat gtgattgatg cccttcctgg |
| 9241 | cgtttgtttt atcctgttat tttctcaaaa aagagcacgg ctttttctta aatacattta |
| 9301 | aattcagaat tgcttgcaaa agattggcct ggatagatag acatcctaga tttaagatag |
| 9361 | taccatctta aaagtatgt: gaaaggtaaa ataaatcatt tcaaaatact taactttcct |
| 9421 | aataaggatg ttaggttttc ttctctttca ttagctggcc ataagtcaag gtccttataa |
| 9481 | cactaggaaa ttaccttttt ttactttcaa aattgcatta tagtgaatta ctgtttacaa |
| 9541 | aatacacata ctgtgaacag atataagttt ctgtctttta taaggtttgt agaatgaatg |
| 9601 | tttttttaaa gtgagtatta tatgttaaat atttraagtt ttttataaat actagtaact |
| 9661 | gtttactaat ttttgtttgg tcaaatgctt gtaaatgtag ctgaaagaag atagggagaa |
| 9721 | actgcggatc ccaaactgtt ccctttttca tttcttgaaa tgttaccact acagacattt |
| 9781 | ttttaaggtg aataaacagc tgtgatgtgc tgtacctaaa atcatgttta atcgtataag |
| 9841 | gaaacatttc aatacactta tacaggaaga aaactataga tgaagtacat gtgtgtgatt |
| 9901 | cagtctgatt cacagaattc tgagagtaat atggaataaa acaactccac ttagatgata |
| 9961 | actgaagcat ttcctgcctt gtgaaaattt ggatcttaaa ttgctgttag aatgggaaat |
| 10021 | ttggacactt tatatcattg tataatttca gaatttagtt tctgtatctt ttggaaaaca |
| 10081 | tgattatagc aaaaacatag aaaataatct attactaaaa caccataaat gtaaaactag |
| 10141 | tatgcttggc tgttaactct aaagatgtta cttatgtctg tttttaaaac atgcatgtat |
| 10201 | ttaacaattt tatcatagta ttgtcatgga aaaaataaat atattttctt acatcttaca |
| 10261 | ctgacagact tgggcattat tgggtttttc cccatgttac acacttagga gaagtggctc |
| 10321 | tgctttgttg ttgtattttt ttttctatc ccgtaagaaa tacagtgctt gtaaagcaat |
| 10381 | cacatagcgc tatggatgta tttttaacat ttgagtacat tgctgtgcca ttggtgtaac |
| 10441 | atccatctgt ccattaagtc attattgttt acctgagaag ttagcatcaa aataatcaaa |
| 10501 | tagtgggaat ccaacattag caaataaata ataaatactg gtctttaaag taa |

An amino acid sequence for the protein (lymphocyte-specific protein tyrosine kinas), also known as human cytokine synthesis inhibitory factor (CSIF)) encoded by the LCK gene that is a regulator of T cell exhaustion/dysfunction resistance is available from the UniPROT database at accession no. P06239 or accession number NP_001036236, NP_001317397 or NP_005347, cDNA NM_001042771, NM_005356 or NM_001330468 with gene location at Chr 1: 32.25-32.29 Mb.

| (SEQ ID NO: 43) | |
|---|---|
| MGCGCSSHPEDDWMENIDVCENCHYPIVPLDGKGTLLIRNGSEV | |
| RDPLVTYEGSNPPASPLQDNLVIALHSYEPSHDGDLGFEKGEQLRILEQSGEWWKAQS | |
| LTTGQEGFIPFNFVAKANSLEPEPWFFKNLSRKDAERQLLAPGNTHGSFLIRESESTA | |
| GSFSLSVRDFDQNQGEVVKHYKIRNLDNGGFYISPRITFPGLHELVRHYTNASDGLCT | |
| RLSRPCQTQKPQKPWWEDEWEVPRETLKLVERLGAGQFGEVWMGYYNGHTKVAVKSLK | |
| QGSMSPDAFLAEANLMKQLQHQRLVRLYAVVTQEPIYIITEYMENGSLVDFLKTPSGI | |
| KLTINKLLDMAAQIAEGMAFIEERNYIHRDLRAANILVSDTLSCKIADFGLARLIEDN | |
| EYTAREGAKFPIKWTAPEAINYGTFTIKSDVWSFGILLTEIVTHGRIPYPGMTNPEVI | |
| QNLERGYRMVRPDNCPEELYQLMRLCWKERPEDRPTFDYLRSVLEDFFTATEGQYQPQ | |
| P | |
| (SEQ ID NO: 44) | |
| 1 | acttgtagcc tgagggctca gagggagcac cggtttggag ctgggacccc ctattttagc |
| 61 | ttttctgtgg ctggtgaatg gggatcccag gatctcacaa tctcagggac catgggctgt |
| 121 | ggctgcagct cacacccgga agatgactgg atggaaaaca tcgatgtgtg tgagaactgc |
| 181 | cattatccca tagtcccact ggatggcaag ggcacgctgc tcatccgaaa tggctctgag |
| 241 | gtgcgggacc cactggttac ctacgaaggc tccaatccgc cggcttcccc actgcaagac |
| 301 | aacctggtta tcgctctgca cagctatgag ccctctcacg acggagatct gggctttgag |
| 361 | aagggggaac agctccgcat cctggagcag agcggcgagt ggtggaaggc gcagtccctg |
| 421 | accacgggcc aggaaggctt catccccttc aattatgtgg ccaaagcgaa cagcctggag |
| 481 | cccgaaccct ggttcttcaa gaacctgagc cgcaaggacg cggagcggca gctcctggcg |
| 541 | cccgggaaca ctcacggctc cttcctcatc cgggagagcg agagcaccgc gggatcgttt |
| 601 | tractgtcgg tccgggact= cgaccagaac cagggagagg ttgtgaaaca ttacaagatc |
| 661 | cgtaatctgg acaacggtgg cttctacatc tcccctcgaa tcacttttcc cggcctgcat |
| 721 | gaactggtcc gccattacac caatgcttca gatgggctgt gcacacggtt gagccgcccc |
| 781 | tgccagaccc agaagcccca gaagccgtgg tgggaggacg agtgggaggt tcccagggag |
| 841 | acgctgaagc tggtggagcg gctgggggct ggacagttcg gggaggtgtg gatggggtac |
| 901 | tacaacgggc acacgaaggt ggcggtgaag agcctgaagc agggcagcat gtccccggac |
| 961 | gccttcctgg ccgaggccaa cctcatgaag cagctgcaac accagcggct ggttcggctc |
| 1021 | tacgctgtgg tcacccagga gcccatctac atcatcactg aatacatgga gaatgggagt |
| 1081 | ctagtggatt ttctcaagac cccttcaggc atcaagttga ccatcaacaa actcctggac |
| 1141 | atggcagccc aaattgcaga aggcatggca ttcattgaag agcggaatta tattcatcgt |
| 1201 | gaccttcggg ctgccaacat tctggtgtct gacaccctga gctgcaagat tgcagacttt |
| 1261 | ggcctagcac gcctcattga ggacaacgag tacacagcca gggagggggc caagtttccc |
| 1321 | attaagtgga cagcgccaga agccattaac tacgggacat tcaccatcaa gtcagatgtg |
| 1381 | tggtcttttg ggatcctgct gacggaaatt gtcacccacg gccgcatccc ttacccaggg |
| 1441 | atgaccaacc cggaggtgat tcagaacctg gagcgaggct accgcatggt gcgccctgac |
| 1501 | aactgtccag aggagctgta ccaactcatg aggctgtgct ggaaggagcg cccagaggac |
| 1561 | cggcccacct ttgactacct gcgcagtgtg ctggaggact tcttcacggc cacagagggc |
| 1621 | cagtaccagc ctcagccttg agaggccttg agaggccctg gggttctccc cctttctctc |
| 1681 | cagcctgact tggggagatg gagttcttgt gccatagtca catggcctat gcacatatgg |
| 1741 | actctgcaca tgaatcccac ccacatgtga cacatatgca ccttgtgtct gtacacgtgt |
| 1801 | cctgtagttg cgtggactct gcacatgtct tgtacatgtg tagcctgtgc atgtatgtct |
| 1861 | tggacactgt acaaggtacc cctttctggc tctcccattt cctgagacca cagagagagg |
| 1921 | ggagaagcct gggattgaca gaagcttctg cccacctact tttctttcct cagatcatcc |
| 1981 | agaagttcct caagggccag gactttatct aatacctctg tgtgctcctc cttggtgcct |
| 2041 | ggcctggcac acatcaggag ttcaataaat gtctgttgat gactgttgta |

An amino acid sequence for the protein (Tyrosine-protein kinase Lyn) encoded by the LYN gene that is a regulator of T cell exhaustion/dysfunction resistance is available from the UniPROT database at accession no. P07948 or accession number NP 001104567 or NP 002341, cDNA NM_001111097 or NM_002350 with gene location at Chr 8: 55.88-56.01 Mb.

| (SEQ ID NO: 45) | |
|---|---|
| MGCIKSKGKDSLSDDGVDLKTQPVRNTERTIYVRDPTSNKQQRP | |
| VPESQLLPGQRFQTKDPEEQGDIVVALYPYDGIHPDDLSFKKGEKMKVLEEHGEWWKA | |
| KSLLTKKEGFIPSNYVAKLNTLETEEWFFKDITRKDAERQLLAPGNSAGAFLIRESET | |
| LKGSFSLSVRDFDPVHGDVIKHYKIRSLDNGGYYISPRITFPCISDMIKHYQKQADGL | |
| CRRLEKACISPKPQKPWDKDAWEIPRESIKLVKRLGAGQFGEVWMGYYNNSTKVAVKT | |
| LKPGTMSVQAFLEEANLMKTLQHDKLVRLYAVVTREEPIYIITEYMAKGSLLDFLKSD | |
| EGGKVLLPKLIDFSAQIAEGMAYIERKNYIHRDLRAANVLVSESLMCKIADFGLARVI | |
| EDNEYTAREGAKFPIKWTAPEAINFGCFTIKSDVWSFGILLYEIVTYGKIPYPGRTNA | |
| DVMTALSQGYRMPRVENCPDELYDIMKMCWKEKAEERPTFDYLQSVLDDFYTATEGQY | |
| QQQP | |
| (SEQ ID NO: 46) | |
| 1 | agttcctctc ccgccgcgcc gggccgcgct gccgctcgct ccccggccgt ggcgcctccg |
| 61 | ggccagacgc gctgcagcct ccagcccgcg gcaagcgggg cggccgcgcc acccccggcc |
| 121 | ccgcgccagc agcccctcgc cgcgcgtcca gcgttcccgg ccagcagcct ccccatacgc |
| 181 | aggtcctgct gggccgcccc gtcgcgcccc ccactctgaa ctcaagtcac cgtggagctc |
| 241 | cgccgccccg aaactttcac cgcgagcggg aaatatggga tgtataaaat caaaagggaa |
| 301 | agacagcttg agtgacgatg gagtagattt gaagactcaa ccagtacgta atactgaaag |
| 361 | aactatttat gtgagagatc caacgtccaa taaacagcaa aggccagttc cagaatctca |
| 421 | gcttttacct ggacagaggt ttcaaactaa agatccagag gaacaaggag acattgtggt |
| 481 | agccttgtac ccctatgatg gcatccaccc ggacgacttg tctttcaaga aaggagagaa |
| 541 | gatgaaagtc ctggaggagc atggagaatg gtggaaagca aagtcccttt taacaaaaaa |
| 601 | agaaggcttc atccccagca actatgtggc caaactcaac accttagaaa cagaagagtg |
| 661 | gtttttcaag gatataacca ggaaggacgc agaaaggcag cttttggcac caggaaatag |
| 721 | cgctggagct ttccttatta gagaaagtga aacattaaaa ggaagcttct ctctgtctgt |
| 781 | cagagacttt gaccctgtgc atggtgatgt tattaagcac tacaaaatta gaagtctgga |
| 841 | taatgggggc tattacatct ctccacgaat cactcttccc tgtatcagcg acatgattaa |
| 901 | acattaccaa aagcaggcag atggcttgtg cagaagattg gagaaggctt gtattagtcc |
| 961 | caagccacag aagccatggg ataaagatgc ctgggagatc ccccgggagt ccatcaagtt |
| 1021 | ggtgaaaagg cttggcgctg ggcagtttgg ggaagtctgg atgggttact ataacaacag |
| 1081 | taccaaggtg gctgtgaaaa ccctgaagcc aggaactatg tctgtgcaag ccttcctgga |
| 1141 | agaagccaac ctcatgaaga ccctgcagca tgacaagctc gtgaggctct acgctgtggt |
| 1201 | caccagggag gagcccattc acatcatcac cgaguacatc gccaagggca gtttgctgga |
| 1261 | tttcctgaag agcgatgaag gtggcaaagt gctgcttcca aagctcattg acttttctgc |
| 1321 | tcagattgca gagggaatgg catacatcga gcggaagaac tacattcacc gggacctgcg |
| 1381 | agcagctaat gttctggtct ccgagtcact catgtgcaaa attgcagatt ttggccttgc |
| 1441 | tagagtaatt gaagataatg agtacacagc aagggaaggt gctaagttcc ctattaagtg |
| 1501 | gacggctcca gaagcaatca actttggatg tttcactatt aagtctgatg tgtggtcctt |
| 1561 | tggaatcctc ctatacgaaa ttgtcaccta tgggaaaatt ccctacccag ggagaactaa |
| 1621 | tgccgacgtg atgaccgccc tgtcccaggg ctacaggatg ccccgtgtgg agaactgccc |
| 1681 | agatgagctc tatgacatta tgaaaatgtg ctggaaagaa aaggcagaag agagaccaac |
| 1741 | gtttgactac ttacagagcg tcctggatga tttctacaca gccacggaag ggcaatacca |
| 1801 | gcagcagcct tagagcacag ggagacccgt ccatttggca ggggtggctg cctcatttag |
| 1861 | agaggaaaag taaccatcac tggttgcact tatgatttca tgtgcgggga tcatctgccg |
| 1921 | tgcctggatc ctgaaataga ggctaaatta ctcaggaaga acaccctcta aatgggaaag |
| 1981 | tattctgtac tcttagatgg attctccact cagttgcaac ttggacttgt cctcagcagc |
| 2041 | tggtaatctt gctctgcttg acaacatctg agtgcagccg tttgagaaga aaacatctat |
| 2101 | tctctccaaa aatgcaccca actagctcta tgtttacaaa tggacatagg actcaaagtt |
| 2161 | tcagagacca ttgcaatgaa tccccaataa ttgcagaact aaactcattt ataaagctaa |
| 2221 | aataaccgga tatatacata gcatgacatt tctttgtgct ttggcttact tgtttaaaaa |
| 2281 | aaaaaaaaaa ctaatccaac ctgttagatt ttgcaggtga agtcagcagc ttaaaaatgt |
| 2341 | ctttcccaga tttcaatgat ttttttcccc ctacctccca aaatctgaga ctgttaaaac |
| 2401 | atttttcttc tatgaacact gctcagacct gctagacatg ccataggagt ggcgtgcaca |
| 2461 | tctctctctc ttccagcagg aggagcccgt gagcacgcac agctgccctg tctgctcacc |
| 2521 | cgaaggcacc gggctcacct ggacctccca ggaaagggag aagagcctca gaaactgctc |
| 2581 | tgtgtttaga aggaatatt: ttaagagtcc agctttttca tttccacaat ttcctatatc |
| 2641 | cagatttgtt ttgacaatgt agtttggaag aactaagatt ctaatctctg aagaacctta |
| 2701 | tagggccttc taaaacataa gagtttcctt tgttgcttca aatatttgaa cattatgtta |
| 2761 | aagatcaagt attaatttta gttgtactct agaaagctaa agtgccacat tcggggctat |
| 2821 | ttttatgatt cagcaatctt ttctaaattg tgtagcatgt gtatgagact atttataccc |
| 2881 | aaggatatga aggaacataa gtgactacaa ggctctaata agccacggtg gcaggaggtt |
| 2941 | caagcggttc tgttcactaa atttttctcc tgtaagcttt gaatggaaac ttctgtatca |
| 3001 | catgatgtgt ttcacttatg ctgttgtgta tatacctaat atttctattt ttgattttat |
| 3061 | tttaatacac ctcgtccaat aacatctcaa gctttttatt tgcatttaca ttttcagctg |
| 3121 | tggtcagtgt aaaaattggt catcagctgg gggcggggtg gttagaagtg attcaacaga |
| 3181 | gctacatgct ttaaacttgc ccaagttcta cctccttcct ttgaacattt cagattggag |
| 3241 | aaccaaggag ttgattgcct gaacacctga acatccgttt atgggggcca gatagaattt |
| 3301 | gttttcaaat aggcttaaca ggcatcatta aaatttcatt ctgtgtgttt tgtttaggct |
| 3361 | tgaggtgctt agaagatggg ataaaatatt ctactttttt ctaaatttta actttgtttc |
| 3421 | ctatgtgatt tttttaaatg tcctttctaa aatattctaa aattattgat tcacaagtgc |
| 3481 | catgttcaga actatagaat attactgtta cataatgtct gcacagctgg tcccttgatt |
| 3541 | cagtggtaag gtttttgtgt acacccccct gcttgcattt tatttcagaa ccacaagtat |
| 3601 | tacccaatat gttacatgga gaggaactat aaagaatccc taaggcaaaa agaagtctct |
| 3661 | agaaaatgac tagagctttt ttttttagca taacaaattt atttaaagaa aattattaaa |
| 3721 | tttatcttcg ccttgttttg cttctcccag ttcctcctct tcttgccatt ttccacttgt |
| 3781 | ctttccctcc caatcaagcc tgtgatcctt acctccatgt gggcccttca ccagcttggg |
| 3841 | cctcatctct ggtgtccagc atgtgtggaa gtcacacgtt cccttgatga acagcacaca |
| 3901 | cagtctcctt acttagctat aggtttccag cctccctgtg acagacaggc ataatgaggg |
| 3961 | gctgaatagg tgtttgtagc attttcgggt atccagtggt gtgcaaaatg gctcatgtca |
| 4021 | tcacacctca ggttattgta gagaactgga aagacagaat ccatactccc taccgccaag |
| 4081 | attctgactt agctgttgtg cagcgggaga tgtatgtcag tctattttaa aagcttctcc |
| 4141 | agtcagctag acacagtggc tcatgcctgt aatctcagca ctttgggagg ctaaggcagg |
| 4201 | aggatcgctt gagctcagga gttagacacc agcctgagca atatagcaag tctctacaaa |
| 4261 | aaattttaaa aattagctaa gtgtggtggc acatacctat ggtcccagct actcaggagg |
| 4321 | ctgaggtggg aggattgctt gagcccagga agtcaaggct gcagtgagct atgatcgtgc |
| 1381 | cactgcactc cagcctgggc aagagtgaga ccctgtctca aaaaaaaaaa ccaaaaaaac |
| 4441 | aaaaaaaccc ttccctggtg aatataatgt atgctcagtc ttgagagtca ccatttggtt |
| 4501 | tgacccatat tacaaagccc agccttcttt ttcctcagat ggtttcattt ctcaaagggg |
| 4561 | agtggaagag aaggaaggag gaaaaaaacg tgccttcctt cctggtgaac atgaagctca |
| 4621 | ctgaacctaa aggagactca caggctagaa accagtgtcc ttagtagtag gggccacctg |
| 4681 | gggcttccgt ggcttctcca ggcatacctc ctgaggttcc acagaatccc cttgcagaaa |
| 4741 | gtacttctga tgagccactg ggaacgtgta ctgtccaagc ccccttctag gggaaattca |
| 4801 | ctgccacaga agccagcagc cctggctgtc ccaagggtgc ataaaaggat aataactaaa |
| 4861 | aggagggaat taattttgtt gcctacacca gggatagatc gagggcactg aaagcatcat |
| 4921 | aagtataaac tcatctccac gtcagcttgt ggagtctgct ttctcgctct ctcttttttt |
| 4981 | tttttttttg acagagtctc actctgtcac gcaggctaga gttgcagtgg tgcgatctca |
| 5041 | gctcattgca acctccccct ctctggttgg agcgattctt ctcctgcctc agcctcccta |
| 5101 | gtagctggaa ctacaggcac acaccatctc gcctggctaa tttttgtatt tttagtagag |
| 5161 | atggggtttc accatgttag ccaggctggt ctcgaactcc tgacctcagg cgatccgcct |
| 5221 | gcctcggctt cccaaagtgc tgggattaca agtgtgagcc actgtgcccg gtcggggtct |
| 5281 | gctttctctt tattccccgt cctctcacaa actacatcca ggggcggtag ctgctaaaag |
| 5341 | gactcaattc tgcaacctca gcttctgaca ccttcaaata aattaaatgc tggtcctagg |
| 5401 | gctcgctccc cagctcttct gatgtcactg ccaggttcca cagccagttg tcggacatct |
| 5461 | cctgcttcct cagccaagaa cacctctcag atgagatcaa accgcccagc tttcctctta |
| 5521 | gcatcttcct ttattttcca cttctggctg gtggaagaga gatcgtagtt ctctattccc |
| 5581 | tacattttaa ctccacgaat attttagatt aagtgaagtc ttgctgttct tcctgatgga |
| 5641 | aatggtaatt ataaacatac aaagataccc atacacacat tctttactca gggccctctt |
| 5701 | gtgctttggc aaatgataaa aattatctct aactcacaca acgaccctgg aagatctgga |
| 5761 | caatgctatc actatttcac agctgagaga tcgggagcat ttaaagccac agaaggaata |
| 5821 | gattcgcttc gcttcatatt gtttccttgt gaaataaaac cagtgaaaca cc |

An amino acid sequence for the protein (Nucleoplasmin-3) encoded by the NPM3 gene that is a regulator of T cell exhaustion/dysfunction resistance is available from the UniPROT database at accession no. 075607 or accession number NP_008924, cDNA NM_006993 with gene location at Chr 10: 101.78-101.78 Mb.

| (SEQ ID NO: 47) | |
|---|---|
| MAAGTAAALAFLSQESRTRAGGVGGLRVPAPVTMDSFFFGCELS | |
| GHTRSFTFKVEEEDDAEHVLALTMLCLTEGAKDECNVVEVVARNHDHQEIAVPVANLK | |
| LSCQPMLSLDDFQLQPPVTFRLKSGSGPVRITGRHQIVTMSNDVSEEESEEEEEDSDE | |
| EEVELCPILPAKKQGGRP | |
| (SEQ ID NO: 48) | |
| 1 | gagggtcccc ggctttgtgc gtgtcggggg cggagtttga agaaggctct tacagcatgg |
| 61 | ccgccggtac tgcagctgcc ttagcgtttt tgagtcagga gagccgaacg cgggccgggg |
| 121 | gtgtcggggg cctacgggtc ccggccccgg tcactatgga cagttttttc ttcggctgtg |
| 181 | agctctccgg ccacacccgc tccttcacct ttaaggtaga ggaagaggat gatgcggagc |
| 241 | acgtgctggc actaaccatg ctctgcctca ccgagggagc caaagacgag tgtaatgtgg |
| 301 | tagaagttgt ggcccggaac catgaccatc aggagatcgc agtccctgtg gccaacctca |
| 361 | agctgtcctg ccaacccatg ctcagtctgg atgacttcca gctccaacca cctgtaacct |
| 421 | tccgcctgaa gtcgggctct ggccctgtgc ggatcactgg gcggcaccag attgttacga |
| 481 | tgagcaatga tgtttctgag gaggagagcg aggaagagga agaggacagt gatgaggaag |
| 541 | aagttgagct gtgccccatc cttcctgcca aaaagcaggg gggcaggccc tagccctcct |
| 601 | aggtcagctc catgtgccat gcaccgccat gcaccctgtt ccctgacaag tttcaacaat |
| 661 | tgtaaatatt tcttccttga agaggagagc ttgggtgggg gttgggtggg agggacttgg |
| 721 | gtctttggtg ctaggagagg gcctgtgctc cacacagccg tggttttctg attttcacca |
| 781 | tgcccggggc ctcccttccc acctgcctgt gagaattgga ggttagtgcc tgaagctcag |
| 841 | agctacacat ttttaatagt ttttacattt ttggataaag gttgaaataa agtggtgtgg |
| 901 | agtttttgca |

An amino acid sequence for the protein (NAD(P)H dehydrogenase [quinone]1) encoded by the NQO1 gene that is a regulator of T cell exhaustion/dysfunction resistance is available from the UniPROT database at accession no. P15559 or accession number NP_000894, NP 001020604, NP_001020605 or NP_001273066. cDNA NM 001286137, NM_000903, NM_001025433 or NM_001025434 with gene location at Chr 16: 69.71-69.73 Mb.

| (SEQ ID NO: 49) | |
|---|---|
| MVGRRALIVLAHSERTSFNYAMKEAAAAALKKKGWEVVESDLYA | |
| MNFNPIISRKDITGKLKDPANFQYPAESVLAYKEGHLSPDIVAEQKKLEAADLVIFQF | |
| PLQWFGVPAILKGWFERVFIGEFAYTYAAMYDKGPFRSKKAVLSITTGGSGSMYSLQG | |
| IHGDMNVILWPIQSGILHFCGFQVLEPQLTYSIGHTPADARIQILEGWKKRLENIWDE | |
| TPLYFAPSSLFDLNFQAGFLMKKEVQDEEKNKKFGLSVGHHLGKSIPTDNQIKARK | |
| (SEQ ID NO: 50) | |
| 1 | acacgcgact cccacaaggt tgcagccgga gccgcccagc tcaccgagag cctagttccg |
| 61 | gccagggtcg ccccggcaac cacgagccca gccaatcagc gccccggact gcaccagagc |
| 121 | catggtcggc agaagagcac tgatcgtact ggctcactca gagaggacgt ccttcaacta |
| 181 | tgccatgaag gaggctgctg cagcggcttt gaagaagaaa ggatgggagg tggtggagtc |
| 241 | ggacctctat gccatgaact tcaatcccat cattcccaga aaggacatca caggtaaact |
| 301 | gaaggaccct gcgaactttc agtatcctgc cgagtctgtt ctggcttata aagaaggcca |
| 361 | tctgagccca gatattgtgg ctgaacaaaa gaagctggaa gccgcagacc ttgtgatatt |
| 421 | ccagttcccc ctgcagtggc ttggagtccc tgccattctg aaaggctggt ttgagcgagt |
| 481 | gttcatagga gagtttgctt acacttacgc tgccatgtat gacaaaggac ccttccggag |
| 541 | taagaaggca gtgctttcca tcaccactgg tggcagtggc tccatgtact ctctgcaagg |
| 601 | gatccacggg gacatgaatg tcattctctg gccaattcag agtggcattc tgcatttctg |
| 661 | tggcttccaa gtcttagaac ctcaactgac atatagcatt gggcacactc cagcagacgc |
| 721 | ccgaattcaa atcctggaag gatggaagaa acgcctggag aatatttggg atgagacacc |
| 781 | actgtatttt gctccaagca gcctctttga cctaaacttc caggcaggat tcttaatgaa |
| 841 | aaaagaggta caggatgagg agaaaaacaa gaaatttggc ctttctgtgg gccatcactt |
| 901 | gggcaagtcc atcccaactg acaaccagat caaagctaga aaatgagatt ccttagcctg |
| 961 | gatttccttc taacatgtta tcaaatctgg gtatctttcc aggcttccct gacttgcttt |
| 1021 | agtttttaag atttgtgttt ttctttttcc acaaggaata aatgagaggg aatcgactgt |
| 1081 | attcgtgcat ttttggatca tttttaactg attcttatga ttactatcat ggcatataac |
| 1141 | caaaatccga ctgggctcaa gaggccactt agggaaagat gtagaaagat gctagaaaaa |
| 1201 | tgttctttaa aggcatctac acaatttaat tcctcttttt agggctaaag ttttagggta |
| 1261 | cagtttggct aggtatcatt caactctcca atgttctatt aatcacctct ctgtagttta |
| 1321 | tggcagaagg gaattgctca gagaaggaaa agactgaatc tacctgccct aagggactta |
| 1381 | acttgtttgg tagttagcca tctaatgctt gtttatgata tttcttgctt tcaattacaa |
| 1441 | agcagttact aatatgccta gcacaagtac cactcttggt cagcttttgt tgtttatata |
| 1501 | cagtacacag ataccttgaa aggaagagct aataaatctc ttctttgctg cagtcatcta |
| 1561 | cttttttttt aattaaaaaa aatttttttt tgaagcagtc ttgctctgtt acccaggctg |
| 1621 | gagtgcagtg gtgtgatctc ggctcactgc aacctctgcc tcccaggttc cagcaattct |
| 1681 | cctgcctcag cctccctagc agctgggatg acaggcgcct gccatcatgc ctgactaatt |
| 1741 | tttgtatttt tagtagagac ggcgtttcac catgutggcc aggctggtct caaactcctg |
| 1801 | acctcaggtg atccgcctac ctcagcctcc caaagtgctg ggattacagg cgtgatccac |
| 1861 | cacacctggc ccttgcaatc ttctacttta aggtttgcag agataaacca ataaatccac |
| 1921 | accgtacatc tgcaatatga attcaagaaa ggaaatagta ccttcaatac ttaaaaatag |
| 1981 | tcttccacaa aaaatacttt atttctgatc tatacaaatt ttcagaaggt tattttcttt |
| 2041 | atcattgcta aactgatgac ttactatggg atggggtcca gtcccatgac cttggggtac |
| 2101 | aattgtaaac ctagagtttt atcaactttg gtgaacagtt ttggcataat agtcaatttc |
| 2161 | tacttctgga agtcatctca ttccactgtt ggtattatat aattcaagga gaatatgata |
| 2221 | aaacactgcc ctcttgtggt gcattgaaag aagagatgag aaatgatgaa aaggttgcct |
| 2281 | gaaaaatggg agacagcctc ttacttgcca agaaaatgaa gggattggac cgagctggaa |
| 2341 | aacctccttt accagatgct gactggcact ggtggttttt gctctcgaca gtatccacaa |
| 2401 | tagctgacgg ctgggtgttt cagtttgaaa atattttgtt gccttcatct tcactgcaat |
| 2461 | tttgtgtaaa tttctcaaag atctgaatta aataaataaa attcatttct acagacccac |
| 2521 | a |

An amino acid sequence for the protein (Pleckstrin homology domain-containing family H member 3) encoded by the PLEKHH3 gene that is a regulator of T cell exhaustion/dysfunction resistance is available from the UniPROT database at accession no. Q7Z736, cDNA NM_024927 with gene location at Chr 17: 42.67-42.68 Mb.

| (SEQ ID NO: 111) | |
|---|---|
| 1 | agtcccgggg ctgcagggag cgcagcgcgc gcggcccggg ccccggccac cggacgccct |
| 61 | accggacacg accctccctg gagcttggac aactgcccac ctcggacgct gcaccggaca |
| 121 | ctgcccccca cagggctgga cactccaacg gacactactt cccagctccg gacattgctc |
| 181 | cctcctctcc cggggccctg gacgcttttc cccctccccc ctccgggggc ccagacactg |
| 241 | agcccccctc agcgtctggc tagcgtcccc ttgcacccca gcccaggaca ctgcctcctc |
| 301 | cctgtcctct tctcccttct gctcctccca cctcatcttt cccctgctcg ggtcggagcc |
| 361 | ccgccagttc ctccgacccc ctactgctcg gccgcggctg cccgcgcccc catccgcagc |
| 421 | tcctcgcccg gatgcctctc cccgggggac tatggtggct tctctgctgc cgtcgaggct |
| 481 | tcactcttct gcaccgggac tacggggacg gcgagcttag cggggacggg gacgaggacg |
| 541 | aggacgagga aacctttgag ctgcggaccc cgagtccagc gggcggcggg aggggtcccc |
| 601 | tggaagtgac gctgactcag ccagtgagga gcgggcctgt ctccaacagg ctgcagagct |
| 661 | gggaggagac ttggagcctc atcccggaga aagggctgcc ggaggacgac ccggacatcg |
| 721 | ttgtgaaagg ttggctgtac cgggagcccc gcggaggagg ggcgcggccc tggctgcccc |
| 781 | cgcgccgagc ctggtttgtg ctcacgcggg actccctgga tcagttcagc agcagcggga |
| 841 | aaggggcgcg gcgtctcggg agcctcgtgc tcaccagcct gtgctcggtg accggcccag |
| 901 | agcgcaggcg taaggagaca ggtctgtggt cagtgactgt gtctggtcgg aaacacagtg |
| 961 | tccgcctctg ctccccacgc caggcagagg ctgagcgctg gggggtggca ttgcgggaag |
| 1021 | tgatcgcctc caaggcaccc ctggagaccc ccacccagct actgctcagg gacatacagg |
| 1081 | aaagttgcgg ggacccagag gccgttgccc tcatttacct gaggaacccg attctgagac |
| 1141 | acactagtgg agccttgtat gccccactcc tgcccctgcc ctatggagtc agcgccccag |
| 1201 | gtccgggcta tgcacccctg cgcgaggagg cggtgcggct gttcttggcg ctgcaggcgc |
| 1261 | tggagggggc gcggcgcccc gggcccttga tgcagggtgt gctccaaacc tgccgggact |
| 1321 | tgcccgcgct ccgggatgaa ctcttcctgc agctggctaa gcagacctcg ggccctgcag |
| 1381 | gtccccccgg gctcccggct acccaagacc ctgcggccct gcggtactgg caactcctca |
| 1441 | cctgcatgag ctgcaccttc cgacctgggg gagctgtgcg ggggcacctc ctggggcact |
| 1501 | tggagaggac cgagcaggca ctcccggact cggaactggc ggaatatgcg cgcttcatcc |
| 1561 | ggaaagcgct gggccggacg cgcggcagag agctggtgcc ctcgctggcg gagatttccg |
| 1621 | cgttgagcca acggcaggag ctgctgtgta ccgtgcactg tccgggggct ggtgcctgtg |
| 1681 | ctgtggccat cgactcccac accacggcgg gggaggtggc tcgagagctg gtggggcggc |
| 1741 | tgggcttggc ccggagccgc aacgcattcg cgctgtacga gcagcgaggg gcccaggagc |
| 1801 | gagccctggc tggggggacc ctcgtggccg acgtgctcac caggtttgag aacttggccg |
| 1861 | cggaggaagc tgggttggag gactcgcccg actccgggtg gagactatgt ctgcgtcttc |
| 1921 | acggacctct gcaccctgag gggctgtccc cagacggtca cgaactgcct ttcctctttg |
| 1981 | agcaggctca cgctctgctg ctgcggggcc ggccgccccc acccgacgac acgctgcgcg |
| 2041 | ccctggcggc gctgcgcctg cagagcctgc agcgggactt ctctccgcgg gtgcccctgc |
| 2101 | cccgcctgga ccgcctgctc ccgcccccgg ccccgccgcg cgaagacccg ccccgcccga |
| 2161 | cccccaggcc gcccccttcc gctgccctgc tggccggggc gctctggagc ccgggcctgg |
| 2221 | ccaagaggcg ggcggagcgg gcccggcgcg gcggggccgg ccgcactgcg ggaagcattg |
| 2281 | cccgcgaggg aggaggcggc gccggcacgg cagctgccgt gctgggcggc tggaagcggc |
| 2341 | tacggggcat gggccgagct gaggccatgg ccgcctacct ggccctggcg gcgcagtgtc |
| 2401 | cggggttcgg cgctgctcgg tatgacgttc tggagctgag cacggagcct ggtcggggtg |
| 2461 | ctccacagaa gctgtgcctg ggcttgggag ccaaggccat gtccctctcc cggccagggg |
| 2521 | agacggagcc catccacagt gtcagctatg gccatgtggc cgcctgccag ctaatgggcc |
| 2581 | cccacaccct ggccttgagg gtgggagaga gccagctcct cctgcagagc ccccaggtgg |
| 2641 | aagagatcat gcagctggtg aatgcctact tggccaaccc ctcccccgag aggccctgca |
| 2701 | gcagctcttc tcctccatgc caagacctgc cagacacctc ccctcccagc cagcgcccgg |
| 2761 | gcctggacga gccccaggga cagtctggct gcttggggca gctgcaggac tgagcctgcc |
| 2821 | aagaggtcac gacttccctc ctgcctgcag cctgggccag gactgctctg agatttgagg |
| 2881 | gaaacatgga cccttttggc cctgcaggga cagggcacat cccacaccca agggctacaa |
| 2941 | tgggtgtggg taattttcta gtttgtttct taatttattt gtagaagaga agcaaaaaaa |
| 3001 | aaaaaaaagt ttcttattta cacaaa |

(SED ID NO: 51)
MPLPGGLWWLLCCRRGFTLLHRDYGDGELSGDGDEDEDEETFELRTPSPAGGGRGPLEVTLTQPVRSGPVSNRLQSW
EETWSLIPEKGLPEDDPDIVVKGWLYREPRGGGARPWLPPRRAWFVLTRDSLDQFSSSGKGARRLGSLVLTSLCSVT
GPERRRKETGLWSVTVSGRKHSVRLCSPRQAEAERWGVALREVIASKAPLETPTQLLLRDIQESCGDPEAVALIYLR
NPILRHTSGALYAPLLPLPYGVSAPGPGYAPLREEAVRLFLALQALEGARRPGPLMQGVLQTCRDLPALRDELFLQL
AKQTSGPAGPPGLPATQDPAALRYWQLLTCMSCTFRPGGAVRGHLLGHLERTEQALPDSELAEYARFIRKALGRTRG
RELVPSLAEISALSQRQELLCTVHCPGAGACAVAIDSHTTAGEVARELVGRLGLARSRNAFALYEQRGAQERALAGG
TLVADVLTRFENLAAEEAGLEDSPDSGWRLCLRLHGPLHPEGLSPDGHELPFLFEQAHALLLRGRPPPPDDTLRALA
ALRLQSLQRDFSPRVPLPRLDRLLPPPAPPREDPPRPTPRPPPSAALLAGALWSPGLAKRRAERARRGGAGRTAGSI
AREGGGGAGTAAAVLGGWKRLRGMGRAEAMAAYLALAAQCPGFGAARYDVLELSTEPGRGAPQKLCLGLGAKAMSLS
RPGETEPIHSVSYGHVAACQLMGPHTLALRVGESQLLLQSPQVEEIMQLVNAYLANPSPERPCSSSSPPCQDLPDTS
PPSQRPGLDEPQGQSGCLGQLQD

An amino acid sequence for the protein (Phosphatase and tensin homolog (PTEN)) encoded by the PTEN gene that is a regulator of T cell exhaustion/dysfunction resistance is available from the UniPROT database at accession no. P60484 or accession number NP_000305, NP_001291646, NP 001291647 or NP_000305.3, cDNA NM_000314, NM 001304717 or NM_001304718 with gene location at Chr 10: 87.86-87.97 Mb.

| (SEQ ID NO: 53) | |
|---|---|
| MTAIIKEIVSRNKRRYQEDGFDLDLTYIYPNIIAMGFPAERLEG | |
| VYRNNIDDVVRFLDSKHKNHYKIYNLCAERHYDTAKFNCRVAQYPFEDHNPPQLELIK | |
| PFCEDLDQWLSEDDNHVAAIHCKAGKGRTGVMICAYLLHRGKFLKAQEALDFYGEVRT | |
| RDKKGVTIPSQRRYVYYYSYLLKNHLDYRPVALLFHKMMFETIPMFSGGTCNPQFVVC | |
| QLKVKIYSSNSGPTRREDKFMYFEFPQPLPVCGDIKVEFFHKQNKMLKKDKMFHFWVN | |
| TFFIPGPEETSEKVENGSLCDQEIDSICSIERADNDKEYLVLTLTKNDLDKANKDKAN | |
| RYFSPNFKVKLYFTKTVEEPSNPEASSSTSVTPDVSDNEPDHYRYSDTTDSDPENEPF | |
| DEDQHTQITKV | |
| (SEQ ID NO: 54) | |
| 1 | gttctctcct ctcggaagct gcagccatga tggaagtttg agagttgagc cgctgtgagg |
| 61 | cgaggccggg ctcaggcgag ggagatgaga gacggcggcg gccgcggccc ggagcccctc |
| 121 | tcagcgcctg tgagcagccg cgggggcagc gccctcgggg agccggccgg cctgcggcgg |
| 181 | cggcagcggc ggcgtttctc gcctcctctt cgtcttttct aaccgtgcag cctcttcctc |
| 241 | ggcttctcct gaaagggaag gtggaagccg tgggctcggg cgggagccgg ctgaggcgcg |
| 301 | gcggcggcgg cggcacctcc cgctcctgga gcggggggga gaagcggcgg cggcggcggc |
| 361 | cgcggcggct gcagctccag ggagggggtc tgagtcgcct gtcaccattt ccagggctgg |
| 421 | gaacgccgga gagttggtct ctccccttct actgcctcca acacggcggc ggcggcggct |
| 481 | ggcacatcca gggacccggg ccggttttaa acctcccgtg cgccgccgcc gcaccccccg |
| 541 | tggcccgggc tccggaggcc gccggcggag gcagccgttc ggaggattat tcgtcttctc |
| 601 | cccattccgc tgccgccgct gccaggcctc tggctgctga ggagaagcag gcccagtcgc |
| 661 | tgcaaccatc cagcagccgc cgcagcagcc attacccggc tgcggtccag agccaagcgg |
| 721 | cggcagagcg aggggcatca gctaccgcca agtccagagc catttccatc ctgcagaaga |
| 781 | agccccgcca ccagcagctt ctgccatctc tctcctcctt tttcttcagc cacaggctcc |
| 841 | cagacatgac agccatcatc aaagagatcg ttagcagaaa caaaaggaga tatcaagagg |
| 901 | atggattcga cttagacttg acctatattt atccaaacat tattgctatg ggatttcctg |
| 961 | cagaaagact tgaaggcgta tacaggaaca atattgatga tgtagtaagg tttttggatt |
| 1021 | caaagcataa aaaccattac aagatataca atctctgtgc tgaaagacat tatgacaccg |
| 1081 | ccaaatttaa ttgcagagtt gcacaatatc ctttcgaaga ccataaccca ccacagctag |
| 1141 | aacttatcaa acccttttgt gaagatcttg accaatggct aagtgaagat gacaatcatg |
| 1201 | ttgcagcaat tcactgtaaa gctggaaagg gacgaactgg tgtaatgata tgtgcatatt |
| 1261 | tattacatag gggcaaatt= ttaaaggcac aagaggccct agatttctat ggggaagtaa |
| 1321 | ggaccagaga caaaaaggga gtaactattc ccagtcagag gcgctatgtg tattattata |
| 1381 | gctacctgtt aaagaatcat ctggattata gaccagtggc actgttgttt cacaagatga |
| 1441 | tgtttgaaac tattccaatg ttcagtggcg gaacttgcaa tcctcagttt gtggtctgcc |
| 1501 | agctaaaggt gaagatatat tcctccaatt caggacccac acgacgggaa gacaagttca |
| 1561 | tgtactttga gttccctcag ccgttacctg tgtgtggtga tatcaaagta gagttcttcc |
| 1621 | acaaacagaa caagatgcta aaaaaggaca aaatgtttca cttttgggta aatacattct |
| 1681 | tcataccagg accagaggaa acctcagaaa aagtagaaaa tggaagtcta tgtgatcaag |
| 1741 | aaatcgatag catttgcagt atagagcgtg cagataatga caaggaatat ctagtactta |
| 1801 | ctttaacaaa aaatgatctt gacaaagcaa ataaagacaa agccaaccga tacttttctc |
| 1861 | caaattttaa ggtgaagctg tacttcacaa aaacagtaga ggagccgtca aatccagagg |
| 1921 | ctagcagttc aacttctgta acaccagatg ttagtgacaa tgaacctgat cattatagat |
| 1981 | attctgacac cactgactct gatccagaga atgaaccttt tgatgaagat cagcatacac |
| 2041 | aaattacaaa agtctgaatt tttttttatc aagagggata aaacaccatg aaaataaact |
| 2101 | tgaataaact gaaaatggac cttttttttt ttaatggcaa taggacattg tgtcagatta |
| 2161 | ccagttatag gaacaattct cttttcctga ccaatcttgt tttaccctat acatccacag |
| 2221 | ggttttgaca cttgttgtcc agttgaaaaa aggttgtgta gctgtgtcat gtatatacct |
| 2281 | ttttgtgtca aaaggacatt taaaattcaa ttaggattaa taaagatggc actttcccgt |
| 2341 | tttattccag ttttataaaa agtggagaca gactgatgtg tatacgtagg aattttttcc |
| 2401 | ttttgtgttc tgtcaccaac tgaagtggct aaagagcttt gtgatatact ggttcacatc |
| 2461 | ctaccccttt gcacttgtgg caacagataa gtttgcagtt ggctaagaga ggtttccgaa |
| 2521 | gggttttgct acattctaat gcatgtattc gggttagggg aatggaggga atgctcagaa |
| 2581 | aggaaataat tttatgctgg actctggacc atataccatc tccagctatt tacacacacc |
| 2641 | tttctttagc atgctacagc tattaatctg gacattcgag gaattggccg ctgtcactgc |
| 2701 | ttgttgtttg cgcatttttt tttaaagcat attggtgcta gaaaaggcag ctaaaggaag |
| 2761 | tgaatctgta ttggggtaca ggaatgaacc ttctgcaaca tcttaagatc cacaaatgaa |
| 2821 | gggatataaa aataatgtca taggtaagaa acacagcaac aatgacttaa ccatataaat |
| 2881 | gtggaggcta tcaacaaaga atgggcttga aacattataa aaattgacaa tgatttatta |
| 2941 | aatatgtttt ctcaattgta acgacttctc catctcctgt gtaatcaagg ccagtgctaa |
| 3001 | aattcagatg ctgttagtac ctacatcagt caacaactta cacttatttt actagttttc |
| 3061 | aatcataata cctgctgtgg atgcttcatg tgctgcctgc aagcttcttt tttctcatta |
| 3121 | aatataaaat attttgtaat gctgcacaga aattutcaat ttgagattct acagtaagcg |
| 3181 | ttttttttct ttgaagattt atgatgcact tattcaatag ctgtcagccg ttccaccctt |
| 3241 | ttgaccttac acattctatt acaatgaatt ttgcagtttt gcacattttt taaatgtcat |
| 3301 | taactgttag ggaattttac ttgaatactg aatacatata atgtttatat taaaaaggac |
| 3361 | atttgtgtta aaaaggaaat tagagttgca gtaaactttc aatgctgcac acaaaaaaaa |
| 3421 | gacatttgat ttttcagtag aaattgtcct acatgtgctt tattgatttg ctattgaaag |
| 3481 | aatagggttt tttttttttt tttttttttt ttttttaaat gtgcagtgtt gaatcatttc |
| 3541 | ttcatagtgc tcccccgagt tgggactagg gcttcaattt cacttcttaa aaaaaatcat |
| 3601 | catatatttg atatgcccag actgcatacg atttraagcg gagtacaact actattgtaa |
| 3661 | agctaatgtg aagatattat taaaaaggtt tttttttcca gaaatttggt gtcttcaaat |
| 3721 | tataccttca ccttgacatt tgaatatcca gccattttgt ttcttaatgg tataaaattc |
| 3781 | cattttcaat aacttattgg tgctgaaatt gttcactagc tgtggtctga cctagttaat |
| 3841 | ttacaaatac agattgaata ggacctacta gagcagcatt tatagagttt gatggcaaat |
| 3901 | agattaggca gaacttcatc taaaatattc ttagtaaata atgttgacac gttttccata |
| 3961 | ccttgtcagt ttcattcaac aatttttaaa tttttaacaa agctcttagg atttacacat |
| 4021 | ttatatttaa acattgatat atagagtatt gattgattgc tcataagtta aattggtaaa |
| 4081 | gttagagaca actattctaa cacctcacca ttgaaattta tatgccacct tgtctttcat |
| 4141 | aaaagctgaa aattgttacc taaaatgaaa atcaacttca tgttttgaag atagttataa |
| 4201 | atattgttct ttgttacaat ttcgggcacc gcatattaaa acgtaacttt attgttccaa |
| 4261 | tatgtaacat ggagggccag gtcataaata atgacattat aatgggcttt tgcactgtta |
| 4321 | ttatttttcc tttggaatgt gaaggtctga atgagggttt tgattttgaa tgtttcaatg |
| 4381 | tttttgagaa gccttgctta cattttatgg tgtagtcatt ggaaatggaa aaatggcatt |
| 4441 | atatatatta tatatataaa tatatattat acatactctc cttactttat ttcagttacc |
| 4501 | atccccatag aatttgacaa gaattgctat gactgaaagg ttttcgagtc ctaattaaaa |
| 4561 | ctttatttat ggcagtattc ataattagcc tgaaatgcat tctgtaggta atctctgagt |
| 4621 | ttctggaata ttttcttaga ctttttggat gtgcagcagc ttacatgtct gaagttactt |
| 4681 | gaaggcatca cttttaagaa agcttacagt tgggccctgt accatcccaa gtcctttgta |
| 4741 | gctcctcttg aacatgtttg ccatactttt aaaagggtag ttgaataaat agcatcacca |
| 4801 | ttctttgctg tggcacaggt tataaactta agtggagttt accggcagca tcaaatgttt |
| 4861 | cagctttaaa aaataaaagt agggtacaag tttaatgttt agttctagaa attttgtgca |
| 4921 | atatgttcat aacgatggct gtggttgcca caaagtgcct cctttacctt taaatactgt |
| 4981 | taatgtgtca tgcatgcaga tggaaggggt ggaactgtgc actaaagtgg gggctttaac |
| 5041 | tgtagtattt ggcagagttg ccttctacct gccagttcaa aagttcaacc tgttttcata |
| 5101 | tagaatatat atactaaaaa atttcagtct gttaaacagc cttactctga ttcagcctct |
| 5161 | tcagatactc ttgtgctgtg cagcagtggc tctgtgtgta aatgctatgc actgaggata |
| 5221 | cacaaaaata ccaatatgat gtgtacagga taatgcctca tcccaatcag atgtccattt |
| 5281 | gttattgtgt ttgttaacaa ccctttatct cttagtgtta taaactccac ttaaaactga |
| 5341 | ttaaagtctc attcttgtca ttgtgtgggt gtttrattaa atgagagttt ataattcaaa |
| 5401 | ttgcttaagt ccattgaagt tttaattaat gggcagccaa atgtgaatac aaagttttca |
| 5461 | gttttttttt ttcctgctgt ccttcaaagc ctactgttta aaaaaaaaaa aaaaaaaaaa |
| 5521 | catggcctga gagtagagta tctgtctact catgcttaat taaggaaaaa cacttatttt |
| 5581 | tagggcttta gtcatcactt cataaattgt ataagcacat taaatagcgt tctagtcctg |
| 5641 | aaaaagtcca agattcttag aaaattgtgc atatttttat tatgacagat gtttgaagat |
| 5701 | aattccccag aatggatttg atactttaga tttcaatttt gtggcttttg tctattattc |
| 5761 | tgtactctgc catcagcata tggaaagctt cattractca tcatgacttg tgccatataa |
| 5821 | aaattgatat ttcggaatag tctaaaggac tttttgtact tgaatttaat catgttgttt |
| 5881 | ctaatattct taaaagcttg aagactaaag catatccttt caacaaagca tagtaaggta |
| 5941 | ataagaaagt gtagtttgta caagtgttaa aaaaataaag tagacaatgt tacagtggga |
| 6001 | cttattattt caagtttaca ttttctccat gtaatttttt aaaaagtaaa tgaaaaaatg |
| 6061 | tgcaataatg taaaatatga agtgtatgtg tacacacatt ttatttttcg gtatcttggg |
| 6121 | tatacgtatg gttgaaaact atactggagt ctaaaagtat tctaatttat aagaagacat |
| 6181 | tttggtgatg tttgaaaaat agaaatgtgc tagttttgtt tttatatcat gtcctttgta |
| 6241 | cgttgtaata tgagctggct tggttcagta aatgccatca ccatttccat tgagaattta |
| 6301 | aaactcacca gtgtttaata tgcaggcttc caaaggctta tgaaaaaaat caagaccctt |
| 6361 | aaatctagtt aatttgctgc taacatgaaa ctctttggtt cttttatttt tgccagataa |
| 6421 | ttagacacac atctaaagct tagtcttaaa tggcttaagt gtagctattg attagtgctg |
| 6481 | ttgctagttc agaaagaaat gtttgtgaat ggaaacaaga atattcagtc caaactgttg |
| 6541 | taaggacagt acctgaaaac caggaaacag gataatggaa aaagtctttt aaagatgaaa |
| 6601 | tgttggagcc aactttctta tagaattaat tgtatgtggc tatagaaagc ctaatgattg |
| 6661 | ttgcttattt ttgagagcat attattcttt tatgaccata atcttgctgt ttttccatct |
| 6721 | tccaaaagat cttccttcta atatgtatat cagaatgtgg gtagccagtc agacaaattc |
| 6781 | atattggttg gtagctttaa aaagtttgta atgtgaagac aggaaaggac aaaatagttt |
| 6841 | gctttggtgg tagtactctg gttgttaagc taggtatttt gagactactt ccccatcaca |
| 6901 | acaacaataa aataatcact cataatccta tcacctggag acatagccat cgttaatatg |
| 6961 | ttagtgacta tacaatcatg ttttcttctg tatatccatg tatattcttt aaaaatgaaa |
| 7021 | tttatactgt acctgatctc aaagcttttt agcttagtat atctgtcatg aatttgtagg |
| 7081 | atgttccatt gcatcagaaa acggacagtg atttgattac tttctaatgc cacagatgca |
| 7141 | gattacatgt agttattgag aatcctttcg aattcagtgg cttaatcatg aatgtctaaa |
| 7201 | tattgttgac attaggatga tacatgtaaa ttaaagttac atttgtttag catagacaag |
| 7261 | cttaacattg tagatgtttc tcttcaaaaa tcatcttaaa catttgcatt tggaattgtg |
| 7321 | ttaaatagaa tgtgtgaaac actgtattag taaacttcat cacctttcta cttccttata |
| 7381 | gtttgaactt ttcagttttt gtagttccca aacagttgct caatttagag caaattaatt |
| 7441 | taacacctgc caaaaaaagg ctgctgttgg cttatcagtt gtctttaaat tcaaatgctc |
| 7501 | atgtgacttt tatcacatca aaaaatattt cattaatgat tcacctttag ctctgaaaat |
| 7561 | taccgcgttt agtaattata gtgggcttat aaaaacatgc aactcttttt gatagttatt |
| 7621 | tgagaatttt ggtgaaaaat atttagctga gggcagtata gaacttataa accaatatat |
| 7681 | tgatattttt aaaacatttt tacatataag taaactgcca tctttgagca taactacatt |
| 7741 | taaaaataaa gctgcatatt tttaaatcaa gtgtttaaca agaatttata ttttttattt |
| 7801 | tttaaaatta aaaataattt atatttcctc tgttgcatga ggattctcat ctgtgcttat |
| 7861 | aatggttaga gattttatt: gtgtggaatg aagtgaggct tgtagtcatg gttctagtgt |
| 7921 | ttcagtttgc caagtctgtt tactgcagtg aaattcatca aatgtttcag tgtggttttc |
| 7981 | tgtagcctat catttactgg ctattttttt atgtacacct ttaggatttt ctgcctactc |
| 8041 | tatccagttg tccaaatgat atcctacatt ttacaaatgc cctttcagtt tctattttct |
| 8101 | ttttccatta aattgccctc atgtcctaat gtgcagtttg taagtgtgtg tgtgtgtgtc |
| 8161 | tgtgtgtgtg tgaatttgat tttcaagagt gctagacttc caatttgaga gattaaataa |
| 8221 | tttaattcag gcaaacatt: ttcattggaa tttcacagtt cattgtaatg aaaatgttaa |
| 8281 | tcctggatga cctttgacat acagtaatga atcttggata ttaatgaatt tgttagtagc |
| 8341 | atcttgatgt gtgttttaat gagttatttt caaagttgtg cattaaacca aagttggcat |
| 8401 | actggaagtg tttatatcaa gttccatttg gctactgatg gacaaaaaat agaaatgcct |
| 8461 | tcctatggag agtatttttc ctttaaaaaa ttaaaaaggt taattatttt gacta |

An amino acid sequence for the protein (Refilin-B) encoded by the RFLB gene that is a regulator of T cell exhaustion/dysfunction resistance is available from the UniPROT database at accession no. Q8N5W9 or accession number NP_874364, cDNA NM_182705.3 with gene location at chromosome=“17”/map=“17p13.3”.

| (SEQ ID NO: 55) | |
|---|---|
| MVGRLSLQDVPELVDAKKKGDGVLDSPDSGLPPSPSPSHWGLAAGGGGGERAAAPGTLEPDAAAATPAAPSPASLPL | |
| APGCALRLCPLSFGEGVEFDPLPPKEVRYTSLVKYDSERHFIDDVQLPLGLAVASCSQTVTCVPNGTWRNYKAEVRF | |
| EPRHRPTRFLSTTIVYPKYPKAVYTTTLDYNCRKTLRRFLSSVELEAAELPGSDDLSDEC | |
| (SEQ ID NO: 56) | |
| 1 | gccggggcgg gcagcggcga gacccgggcg gcgcgcgggc ggcggggcag ggagggaggt |
| 61 | agggcggccg ggcgggcagc ggccccggcc cgcgtctgcg cccctcgctg tcccgcgacc |
| 121 | ccggcgcggg tgcctcgggc ccccctcgcg cgccgccatg gtgggccggc tgagcctaca |
| 181 | ggatgtgccc gagctcgtgg acgcgaagaa gaagggcgac ggcgtcctgg acagcccgga |
| 241 | ctcggggctg ccccccagcc ccagccccag ccactggggg ctcgcggcgg gcggaggcgg |
| 301 | cggagagcgc gcggcggcac cggggacgct ggagcccgac gcggcggcgg cgacccccgc |
| 361 | ggctccgagt ccagcgtctc tccccctggc tcccggctgt gcgctgaggc tttgtcccct |
| 421 | gtcctttggc gaaggagtgg agtttgaccc cttaccacca aaggaagtaa ggtacacctc |
| 481 | cttggtcaag tacgactccg agaggcactt catcgacgac gtgcagctgc ccctgggcct |
| 541 | ggcggtggcc tcctgcagcc agacggtcac ctgtgtcccc aatggcacgt ggcgcaacta |
| 601 | caaggccgag gtgcgcttcg agccacgcca caggcccacc cgcttcctca gtaccaccat |
| 661 | cgtgtacccc aagtacccca aggccgtcta caccaccacc ctggattaca actgccgcaa |
| 721 | gacgctgagg aggtttctgt ccagcgtgga gctcgaagcc gcggagctcc cgggcagcga |
| 781 | cgacctctct gatgaatgct gacccagcag gctgggctgg ggtcggaccg gctctcccgc |
| 841 | tgtcctgccc ccgactgccc tggccaagct gggcgacctt acccttcacg tcattccagc |
| 901 | cccagaggag tctaacctag agatacctcc aagcgcggcc gggggaagag aaaaaaaaga |
| 961 | aaaaatcact gcatctcata attattgaga tctttgtgtt gtaattttca agctgttttt |
| 1021 | agagggaata tatgtcctgg tttgctgctg tgttgtttcc ccaaagtctc aatcagatga |
| 1081 | ggcaacaaaa agaccaccag aattgcagga aagcagcagc agtctgggat agggaggggg |
| 1141 | gaggagagct ctccctccgt ggtcagtttg tcagaagaaa agcatggaaa aagtgagatt |
| 1201 | taagaaatct cagccacggg tgtcttcact gcacaaaagt ggtgctaatt cagtaaacag |
| 1261 | tgacacctgt gtgggttcaa tctgtggaga gttgagttcc attcctttgt ttttaaattt |
| 1321 | ccacctccat ttgtgtttcc tattaaccta aactcttgta attctctaaa tcttttttca |
| 1381 | tgatataaaa aaaaaagaaa atcccaaacc acaatttaag atgccttttt tttttaaaaa |
| 1441 | aaaaggttct ccgtgttgcc acctgctccg ggaaggccag gccgatgggc ggccaggttt |
| 1501 | gggagcagcg gcttcggctc cgattgcttc ctgacggtca gttccaagca caggctctga |
| 1561 | gaaacgcctc gaacccttgg tatctggtga ccgtctccgc aggcttccgt gagtctccga |
| 1621 | taggtgtttt gggcttttgg aatactttca gaatactttc ccattttttc ataaggcaaa |
| 1681 | acggagtgga cggccttttt ttgagagaaa cagcattcta agagtgtgct tggaaaacac |
| 1741 | actttttggg agtccacgtg gggagtggac actcaggaca ggaagcggtg gccctaagac |
| 1801 | ctgcggccac ccggaggagg cagacgcagg cgatgacctg gttcatcatg agaggagcta |
| 1861 | gagttgactc agtcttagga tctcgatggc gtcagaattg cagagggttt ctgccagtgc |
| 1921 | cttaggtcta attcagaaag caaggaaggc tgggcacggt ggctggcgcc tgtagtctca |
| 1981 | gctacttgga aactgaggca ggaggatctc ttgagcccaa gaattcgaga ccggtctggc |
| 2041 | aacatagtga gcctccatch ctaaaaaaaa gtggcaaagc tgaggaaatc tgtgccccca |
| 2101 | tcccccgaca ccagacaagc taaggtgcat tcagagtagg gaacgatgtc aaaacctggc |
| 2161 | cgccgtggtg gcgcgcattc ctctcacacc accgtacgga ggcaggtgac agtcaactga |
| 2221 | aatgtcccca aggaccgtgt gaattcggct ggcgtggccc agcagcgggg tggaccaggg |
| 2281 | tgcggtggct tcagacctgc ctcccgccac cccgtcctcc cctaacccct tcttgggacc |
| 2341 | tcctgccaat ccctgtggtg tttgagtaaa gaaagtggct gatggtttta cttttttctt |
| 2401 | ccaatattgg aaagaacaaa ctagatattc cccttgaaca catccaaatt atttccagtg |
| 2461 | cctgagggtt accgagcact gtaagctcag ctcggctgta gctccgggtt tctgccgatg |
| 2521 | gcctccatct gcgccgaaag ctgaactgag ccccgcggtg ccccggagcc acagcagcag |
| 2581 | gtgcagcgac aatcgccgtc gtgcgcagtc gctggagtcc tggagagcca cgctgtcctc |
| 2641 | tgcagacaac agcatcagtc cgctgtgccc caatgcatcg acgccctgtg gacggggcct |
| 2701 | gcagtccacc cgtggtctga gcttgccggg ctgaagctcg gctgggaccc agggaggggc |
| 2761 | caccctggga cccacagtct cctcagtcac gtgcagagca aaaaatgctt cctgtgtttg |
| 2821 | gaagagcaag tgacgcagca tttattgata aaaatggatt tttctgtctg atttcatgct |
| 2881 | gtggtcgttt aagtggtcaa tgtctttttt tttttttttt ttgacatgga gtctcgctct |
| 2941 | gttgcccagg ctgagtggaa tggcgccaac tcagctcact gcaaccttca cctcccaggc |
| 3001 | tcaagcgact ctcctgcctc agcctcctga gcagctggga ttacaggtgc ccaccaccac |
| 3061 | gcccagctaa tttttgtatt tttagtagag acggggtttc accatgttgg ccaggctggt |
| 3121 | ctcgaacacc tgaccttgtg atccacccgc ctcggcctcc caataatttg taagttatgt |
| 3181 | tagcgggatc ctcaaggcct tgctttgccc cgtggagacg cttgctcgga tgagctcagg |
| 3241 | aaacagtacc ggctgcgtgg caggtctggg tgttgtgtgc gaggacgtgg cctttgaaca |
| 3301 | ccgctgtgtt ctcagaggtc cttaggagat attttttttt gtcttagggg gactgtgtta |
| 3361 | agttcagaca aatcatgctg ggtgtgtaga gagtgtgaaa tacgtcagtg aagtaagtag |
| 3421 | cagtgagcga ttgtgaatgt gtaatgtaaa tggaaaaccg ggttttaccg tgttaagtta |
| 3481 | ttcactaggg agccagtcgt agttctttgt aatcctcttt cttccaaacc tgctttgctg |
| 3541 | aaagttgcag aaaaggaag= gtgtggagag aaacagaacc cttcagggtg ggtcagagga |
| 3601 | cgccatccac agtggattcg tgttcgtttg caggtggaag cagtgatttt taggacccac |
| 3661 | tgattaaaaa caaacattcc caagtgtctc tgagagatgc tgtttatttg ttaattaaaa |
| 3721 | agcttttttc tctgtctttt aaattatggc tttcatgtaa taaggatatt tttagtgaaa |
| 3781 | aattgttttc ctttcaaatt acagaccttt taaaaaaact taatttgagc gagtaccttt |
| 3841 | tcatttgaca cttttcctgt ttctaacctt aggaaaccag aatagcgttt ggcagacacg |
| 3901 | acgttttcag tttacctttg acacctgccc cactccattt tgctttgtga tgtcttcatt |
| 3961 | taacaataaa ttatctgaaa aaacaaaa |

An amino acid sequence for the protein (Signal transducer and activator of transcription 5A) encoded by the STAT5A gene that is a regulator of T cell exhaustion/dysfunction resistance is available from the UniPROT database at accession no. P42229 or accession number NP 001275647, NP_001275648, NP_001275649 or NP_003143, cDNA NM_001288718, NM_001288719, NM_001288720 or NM_003152 with gene location at Chr 17: 42.29-42.31 Mb.

| (SEQ ID NO: 57) | |
|---|---|
| MAGWIQAQQLQGDALRQMQVLYGQHFPIEVRHYLAQWIESQPWD | |
| AIDLDNPQDRAQATQLLEGLVQELQKKAEHQVGEDGFLLKIKLGHYATQLQKTYDRCP | |
| LELVRCIRHILYNEQRLVREANNCSSPAGILVDAMSQKHLQINQTFEELRLVTQDTEN | |
| ELKKLQQTQEYFIIQYQESLRIQAQFAQLAQLSPQERLSRETALQQKQVSLEAWLQRE | |
| AQTLQQYRVELAEKHQKTLQLLRKQQTIILDDELIQWKRRQQLAGNGGPPEGSLDVLQ | |
| SWCEKLAEIIWQNRQQIRRAEHLCQQLPIPGPVEEMLAEVNATITDIISALVTSTFII | |
| EKQPPQVLKTQTKFAATVRLLVGGKLNVHMNPPQVKATIISEQQAKSLLKNENTRNEC | |
| SGEILNNCCVMEYHQATGTLSAHFRNMSLKRIKRADRRGAESVTEEKFTVLFESQFSV | |
| GSNELVFQVKTLSLPVVVIVHGSQDHNATATVLWDNAFAEPGRVPFAVPDKVLWPQLC | |
| EALNMKFKAEVQSNRGLTKENLVFLAQKLFNNSSSHLEDYSGLSVSWSQFNRENLPGW | |
| NYTFWQWFDGVMEVLKKHHKPHWNDGAILGFVNKQQAHDLLINKPDGTFLLRFSDSEI | |
| GGITIAWKFDSPERNLWNLKPFTTRDFSIRSLADRLGDLSYLIYVFPDRPKDEVFSKY | |
| YTPVLAKAVDGYVKPQIKQVVPEFVNASADAGGSSATYMDQAPSPAVCPQAPYNMYPQ | |
| NPDHVLDQDGEFDLDETMDVARHVEELLRRPMDSLDSRLSPPAGLFTSARGSLS | |
| (SEQ ID NO: 58) | |
| 1 | agtaacagcc gccgctgccg ccgccgccag gaaccccggc cgggagcgag agccgcgggg |
| 61 | cgcagagccg gcccggctgc cggacggtgc ggccccacca ggtgaacggc catggcgggc |
| 121 | tggatccagg cccagcagct gcagggagac gcgctgcgcc agatgcaggt gctgtacggc |
| 181 | cagcacttcc ccatcgaggt ccggcactac ttggcccagt ggattgagag ccagccatgg |
| 241 | gatgccattg acttggacaa tccccaggac agagcccaag ccacccagct cctggagggc |
| 301 | ctggtgcagg agctgcagaa gaaggcggag caccaggtgg gggaagatgg gtttttactg |
| 361 | aagatcaagc tggggcacta cgccacgcag ctccagaaaa catatgaccg ctgccccctg |
| 421 | gagctggtcc gctgcatccg gcacattctg tacaatgaac agaggctggt ccgagaagcc |
| 481 | aacaattgca gctctccggc tgggatcctg gttgacgcca tgtcccagaa gcaccttcag |
| 541 | atcaaccaga catttgagga gctgcgactg gtcacgcagg acacagagaa tgagctgaag |
| 601 | aaactgcagc agactcagga gtacttcatc atccagtacc aggagagcct gaggatccaa |
| 661 | gctcagtttg cccagctggc ccagctgagc ccccaggagc gtctgagccg ggagacggcc |
| 721 | ctccagcaga agcaggtgtc tctggaggcc tggttgcagc gtgaggcaca gacactgcag |
| 781 | cagtaccgcg tggagctggc cgagaagcac cagaagaccc tgcagctgct gcggaagcag |
| 841 | cagaccatca tcctggatga cgagctgatc cagtggaagc ggcggcagca gctggccggg |
| 901 | aacggcgggc cccccgaggg cagcctggac gtgctacagt cctggtgtga gaagttggcc |
| 961 | gagatcatct ggcagaaccg gcagcagatc cgcagggctg agcacctctg ccagcagctg |
| 1021 | cccatccccg gcccagtgga ggagatgctg gccgaggtca acgccaccat cacggacatt |
| 1081 | atctcagccc tggtgaccag cacattcatc attgagaagc agcctcctca ggtcctgaag |
| 1141 | acccagacca agtttgcagc caccgtacgc ctgctggtgg gcgggaagct gaacgtgcac |
| 1201 | atgaatcccc cccaggtgaa ggccaccatc atcagtgagc agcaggccaa gtctctgctt |
| 1261 | aaaaatgaga acacccgcaa cgagtgcagt ggtgagatcc tgaacaactg ctgcgtgatg |
| 1321 | gagtaccacc aagccacggg caccctcagt gcccacttca ggaacatgtc actgaagagg |
| 1381 | atcaagcgtg ctgaccggcg gggtgcagag tccgtgacag aggagaagtt cacagtcctg |
| 1441 | tttgagtctc agttcagtgt tggcagcaat gagcttgtgt tccaggtgaa gactctgtcc |
| 1501 | ctacctgtgg ttgtcatcgt ccacggcagc caggaccaca atgccacggc tactgtgctg |
| 1561 | tgggacaatg cctttgctga gccgggcagg gtgccatttg ccgtgcctga caaagtgctg |
| 1621 | tggccgcagc tgtgtgaggc gctcaacatg aaattcaagg ccgaagtgca gagcaaccgg |
| 1681 | ggcctgacca aggagaacct cgtgttcctg gcgcagaaac tcttcaacaa cagcagcagc |
| 1741 | cacctggagg actacagtgg cctgtccgtg tcctggtccc agttcaacag ggagaacttg |
| 1801 | ccgggctgga actacacctt ctggcagtgg tttgacgggg tgatggaggt gttgaagaag |
| 1861 | caccacaagc cccactggaa tgatggggcc atcctaggtt ttgtgaataa gcaacaggcc |
| 1921 | cacgacctgc tcatcaacaa gcccgacggg accttcttgt tgcgctttag tgactcagaa |
| 1981 | atcgggggca tcaccatcgc ctggaagttt gactccccgg aacgcaacct gtggaacctg |
| 2041 | aaaccattca ccacgcggga tttctccatc aggtccctgg ctgaccggct gggggacctg |
| 2101 | agctatctca tctatgtgt: tcctgaccgc cccaaggatg aggtcttctc caagtactac |
| 2161 | actcctgtgc tggctaaagc tgttgatgga tatgtgaaac cacagatcaa gcaagtggtc |
| 2221 | cctgagtttg tgaatgcatc tgcagatgct gggggcagca gcgccacgta catggaccag |
| 2281 | gccccctccc cagctgtgtg cccccaggct ccctataaca tgtacccaca gaaccctgac |
| 2341 | catgtactcg atcaggatgg agaattcgac ctggatgaga ccatggatgt ggccaggcac |
| 2401 | gtggaggaac tcttacgccg accaatggac agtcttgact cccgcctctc gccccctgcc |
| 2461 | ggtcttttca cctctgccag aggctccctc tcatgaatgt ttgaatccca cgcttctctt |
| 2521 | tggaaacaat atgcaatgtg aagcggtcgt gttg gagtt tagtaaggct gtgtacactg |
| 2581 | acacctttgc aggcatgcat gtgcttgtgt gtgtgtgtgt gtgtgtgtcc ttgtgcatga |
| 2641 | gctacgcctg cctcccctgt gcagtcctgg gatgtggctg cagcagcggt ggcctctttt |
| 2701 | cagatcatgg catccaagag tgcgccgagt ctgtctctgt catggtagag accgagcctc |
| 2761 | tgtcactgca ggcactcaat gcagccagac ctattcctcc tgggcccctc atctgctcag |
| 2821 | cagctatttg aatgagatga ttcagaaggg gaggggagac aggtaacgtc tgtaagctga |
| 2881 | agtttcactc cggagtgaga agctttgccc tcctaagaga gagagacaga gagacagaga |
| 2941 | gagagaaaga gagagtgtgt gggtctatgt aaatgcatct gtcctcatgt gttgatgtaa |
| 3001 | ccgattcatc tctcagaagg gaggctgggg ttcattttcg agtagtattt tatactttag |
| 3061 | tgaacgtgga ctccagactc tctgtgaacc ctatgagagc gcgtctgggc ccggccatgt |
| 3121 | ccttagcaca ggggggccgc cggtttgagt gagggtttct gagctgctct gaattagtcc |
| 3181 | ttgcttggct gcttggcctt gggcttcatt caagtctatg atgctgttgc ccacgtttcc |
| 3241 | cgggatatat attctctccc ctccgttggg ccccagcctt ctttgcttgc ctctctgttt |
| 3301 | gtaaccttgt cgacaaagag gtagaaaaga ttgggtctag gatatggtgg gtggacaggg |
| 3361 | gccccgggac ttggagggtt ggtcctcttg cctcctggaa aaaacaaaaa caaaaaactg |
| 3421 | cagtgaaaga caagctgcaa atcagccatg tgctgcgtgc ctgtggaatc tggagtgagg |
| 3481 | ggtaaaagct gatctggttt gactccgctg gaggtggggc ctggagcagg ccttgcgctg |
| 3541 | ttgcgtaact ggctgtgttc tggtgaggcc ttgctcccaa ccccacacgc tcctccctct |
| 3601 | gaggctgtag gactcgcagt caggggcagc tgaccatgga agattgagag cccaaggttt |
| 3661 | aaacttctct gaagggaggt ggggatgaga agaggggttt ttttgtactt tgtacaaaga |
| 3721 | ccacacattt gtgtaaacag tgttttggaa taaaatattt ttttcataaa |

An amino acid sequence for the protein (Leucine-rich repeat (LRR) protein SHOC-2) encoded by the SHOC2 gene that is a regulator of T cell exhaustion/dysfunction resistance is available from at accession number Q9UQ13, cDNA NM_007373 with gene location at Chr 10: 110.92-111.01.

| (SEQ ID NO: 112) | |
|---|---|
| 1 | gaagaggagg aaggagggcg agcgaggagg atggcggagt cggggctcct gacggaactc |
| 61 | taatgaatca ttgattgacc agcactattt taccagttgg aatgaatgat cagaaatggg |
| 121 | catagtgctt ttagatccaa catgtaacag atggatgtta ctccatgctg attacttctt |
| 181 | caagccagta cttttttgat tgtgtaggat ctttgtctct tcatctttga attcaattac |
| 241 | tggaaaataa aaggagttca tgtagttttt gtccaggctt gagtcaccat gagtagtagt |
| 301 | ttaggaaaag aaaaagactc taaagaaaaa gatcccaaag taccatcagc caaggaaaga |
| 361 | gaaaaggagg caaaagcctc tggaggtttt gggaaagaga gcaaagaaaa agaacctaag |
| 421 | accaaaggga aagatgccaa agatggaaag aaggactcca gtgctgccca accaggggtg |
| 481 | gcattttcag ttgacaatac gatcaaacgg ccaaacccag cacctgggac tagaaaaaaa |
| 541 | tccagcaatg cagaggtgat taaagagctc aacaaatgcc gggaagagaa ttcaatgcgt |
| 601 | ttggacttat ccaagagatc tatacacata ttgccatcat caatcaaaga gttgactcaa |
| 661 | ttaacagaac tttatttata cagtaacaaa ttgcagtccc tcccagcaga ggtgggatgt |
| 721 | ttagtaaatc tcatgacact ggctctaagt gaaaattcac ttaccagttt gcctgactct |
| 781 | cttgataact tgaagaagct gcggatgctt gatttacggc ataataaact gagagaaatt |
| 841 | ccttcagtgg tgtataggct ggattctctc accactcttt accttcgctt taatcgtata |
| 901 | actactgtgg aaaaggacat caaaaacttg tcaaaactca gcatgcttag cattcgagag |
| 961 | aacaaaatta aacaactacc tgctgaaatt ggtgaattat gtaacctcat tacgctggat |
| 1021 | gtagctcaca atcaacttga acaccttcca aaggagattg gaaactgtac acagataacc |
| 1081 | aaccttgact tgcagcacaa tgaactgcta gaccccccag atactatagg aaacctgtcc |
| 1141 | agtttaagtc gtcttggtct gagatataac agactgtcag caatacccag atcattagca |
| 1201 | aaatgcagtg cacttgaaga attaaattta gagaacaata acatttctac tttaccagag |
| 1261 | agtcttttat caagtcttgt gaaactgaat agtttgacct tagctagaaa ttgcttccag |
| 1321 | ttgtatccag tgggtggtcc atctcagttt tctaccatct attccctcaa catggaacac |
| 1381 | aatcgaatca acaaaattcc atttggaatt ttctccagag caaaagtatt aagtaagctg |
| 1441 | aatatgaagg acaatcagtt aacatcactt ccctcggatt ttggaacttg gaccagtatg |
| 1501 | gtagaattga atttagccac taatcagctc acaaagatcc ctgaggatgt gtctggtctc |
| 1561 | gtttctcttg aggttcttat cttatcaaac aatcttctaa agaagcttcc ccatggtctt |
| 1621 | ggaaacctta ggaagttaag agagttggat ctagaagaga acaaattgga atccttgcca |
| 1681 | aatgaaattg catatcttaa ggatttacag aaattagtct tgacaaacaa ccagttgacc |
| 1741 | actcttccca gaggcattgg tcaccttact aatctcacac atctgggcct tggagagaac |
| 1801 | ctacttactc accttcctga agaaattggt acactggaga acctagaaga actgtatttg |
| 1861 | aatgacaacc ccaacctgca tagccttccc tttgagctgg cactctgcag caagctttca |
| 1921 | atcatgagta ttgagaactg tccactcagt caccttccac ctcagattgt tgctgggggg |
| 1981 | ccttctttca tcattcagtt cttaaagatg cagggtccat atcgtgccat ggtctgatat |
| 2041 | aaatctgctg gtcccacaca ctgttcaaaa atagactgcc attaatgttt cttatctata |
| 2101 | tctgtatcta tttatgtaga tattggtata tggcagattt ataaaaattg cattatgtgt |
| 2161 | ttctgctaat agaggaatca tagccattta gaattttttt taaattctgt acaaaaggct |
| 2221 | tatataagtt ttctttgctg aatttgatgg atgtttttct gttgtgtaat ctgatatgcc |
| 2281 | agtttgctta aaacatttgc caacacatta tgaagttatt aaatttaagg gacagaggta |
| 2341 | gtatagttag atatactttc tcttaggaaa aataatgggc aaaaattttt gttgcaactt |
| 2401 | ttcatatata ttttcccctt accaattgtt ttatccttat agtattgtag gccctgaaag |
| 2461 | tagaattttt ctttaactta ttttgagatt tgagatttaa attttatgta ttgtttacag |
| 2521 | tcagagtaaa tcactggatt tcttttgttt gttttgattt gctctgtttt attcagtcaa |
| 2581 | atctagagtt tgaatcctct gctaaagaat ttgcatccac tggtgtaaac agtgaaaggt |
| 2641 | atttgcttgt tgaaaaaaaa aactggcaaa gtgaaaagat acagtcaaaa atctagaatt |
| 2701 | tctttaattt tgcttctctg acgagttgtg aagcaaaata cctgaagtga gtctttgggt |
| 2761 | aggggaaggg tattgagacc ttttctagta tgaatatttt ttaagtttgg gggaagagaa |
| 2821 | acttgcagtg aaaaggagtt ttttcattcc tgaaagttgc agatccacaa aactaacagg |
| 2881 | ataattgggc aaataaatta catataaaca cacacaatct atatatgtat atacaatgct |
| 2941 | atatagatat gtatttatta tatcataaac tacagtaggt aactttaagg atttcttcct |
| 3001 | atccttgtac aatgacatga atgtctttct ttgaaaactg caatgtatgt atgtttcaag |
| 3061 | gttatttaac agtgtactat ggttttatat cttgacttgc cttgtacatc tttcaattct |
| 3121 | ggaatatctg tgtctaagca caatatcttc acactgtgct gtattgctgc tgaactaaat |
| 3181 | gcacttttcc ccacatatgg ggcactggct tcaaacaatt cagttcagta tcattacttt |
| 3241 | taatctcatc tttcctttct tggtagttgt taatacagtt atggaaaaga ggcacattgc |
| 3301 | atagaagcca ttggggagt= cagtggaagt tctgraagat gtgcatgtac tatttgatgc |
| 3361 | gttttctttg cttcactgct tttaatactt agcagtattg ttggtctaag tcaatttgat |
| 3421 | tattgaggag tctcagagca aggtgcgttc tagatgtcat cctaaaaaac acttcatata |
| 3481 | taattaatca ctattttgta taattacata ttgctgcttg tgtgtttttt tttttttcca |
| 3541 | tttagttggg cgttgtgttt tacacaaaac cattcttgaa ttaaggctat gatattaaga |
| 3601 | tagaaatttg gactgttgtt ctgcttttcc tggcactcaa attcatgact agttttgagg |
| 3661 | tcaaacctat gttcgtaatg agagatttta taaggatcaa ctaagaaatg gaaggcaggt |
| 3721 | gaagatataa aaccctagaa tgcttaaatg tgctgtaaaa ctattgtaga tgtcactgga |
| 3781 | ttttaccaag taatatcctt tctttttttt ttccccccat ctgctgtggc ttttcagtta |
| 3841 | aaattttgtt tataaaagga atttgtttat tacagctcta ccta |

(SEQ ID NO: 113)
MSSSLGKEKDSKEKDPKVPSAKEREKEAKASGGFGKESKEKEPKTKGKDAKDG
KKDSSAAQPGVAFSVDNTIKRPNPAPGTRKKSSNAEVIKELNKCREENSMRLDLSKRSI
HILPSSIKELTQLTELYLYSNKLQSLPAEVGCLVNLMTLALSENSLTSLPDSLDNLKKLR
MLDLRHNKLREIPSVVYRLDSLTTLYLRFNRITTVEKDIKNLSKLSMLSIRENKIKQLPAE
IGELCNLITLDVAHNQLEHLPKEIGNCTQITNLDLQHNELLDLPDTIGNLSSLSRLGLRYN
RLSAIPRSLAKCSALEELNLENNNISTLPESLLSSLVKLNSLTLARNCFQLYPVGGPSQFS
TIYSLNMEHNRINKIPFGIFSRAKVLSKLNMKDNQLTSLPLDFGTWTSMVELNLATNQL
TKIPEDVSGLVSLEVLILSNNLLKKLPHGLGNLRKLRELDLEENKLESLPNEIAYLKDLQ
KLVLTNNQLTTLPRGIGHLTNLTHLGLGENLLTHLPEEIGTLENLEELYLNDNPNLHSLP
FELALCSKLSIMSIENCPLSHLPPQIVAGGPSFIIQFLKMQGPYRAMV

An amino acid sequence for the protein (S100 calcium-binding protein A13 (S100A13)) encoded by the S100A13 gene that is a regulator of T cell exhaustion/dysfunction resistance is available from the UniPROT database at accession no. Q99584 or accession number NP_001019381, NP_001019382, NP_001019383, NP_001019384 or NP_005970, cDNA NM_001024210, NM_001024211, NM_001024212. NM_001024213 or NM_005979 with gene location at Chr 1: 153.62-153.63 Mb.

| (SEQ ID NO: 61) | |
|---|---|
| MAAEPLTELEESIETVVTTFFTFARQEGRKDSLSVNEFKELVTQ | |
| QLPHLLKDVGSLDEKMKSLDVNQDSELKFNEYWRLIGELAKEIRKKKDLKIRKK | |
| (SEQ ID NO: 62) | |
| 1 | ctcactaccg aaactcaccg aaggaaacag acgccagtgc tcctcccggg gctgccacca |
| 61 | cggctccggc aggcgggccg gggaccggcc gaacctgagt tgacggtgga ggggctcggg |
| 121 | ttagctagat gggcggttgg ttagatgcgt aagcggtagt atgcgagctc agttcgttgt |
| 181 | tgctggttgg ctgtctagtc ggccgatccg tctgctcacc cggcctgccc tttcctgcct |
| 241 | ttcgtctgca gcggccgcca gctcctgctc ggtgtccaaa acaaaatggc cgccacgtcc |
| 301 | agtgcttgtc tgaccggcta aaatggcgtc tacgcaatta cgtcaggcgt cagatccgcg |
| 361 | cacgactaga gggcgggaaa gatttgagct acgcctgcgc agagttgaga agctgatgtc |
| 421 | tcctcgactt ccaactggaa ccttgaaccc ccacatttct ggaccttgag cattcctcaa |
| 481 | gtaggaagat gtaatgcacc ttgacctctt tctaaataag acacttcccc cattcctcaa |
| 541 | agttgagagt gaacagtctt cacgtctcca cccacttcca gatcccagag gagacagaca |
| 601 | tcggatggct gttacctcct cctaaaatgt ctgcatttac ccagataatc ttccaatgga |
| 661 | aatccatggt tcaagtgcca cctcttcagg aaagccatct gacttcaatc aggtcagccc |
| 721 | tgacaaaggt cagctagccc cttgaggaca tcagctttgg cctcagggtc ctaatggcag |
| 781 | cagaaccact gacagagcta gaggagtcca ttgagaccgt ggtcaccacc ttcttcacct |
| 841 | ttgcaaggca ggagggccgg aaggatagcc tcagcgtcaa cgagttcaaa gagctggtta |
| 901 | cccagcagtt gccccatctg ctcaaggatg tgggctctct tgatgagaag atgaagagct |
| 961 | tggatgtgaa tcaggactcg gagctcaagt tcaatgagta ctggagattg attggggagc |
| 1021 | tggccaagga aatcaggaag aagaaagacc tgaagatcag gaagaagtaa agccgcctgg |
| 1081 | ctgagatggg gtgggcaggg cagagctgat cagggccgag cagaaccgca ctcttcccaa |
| 1141 | ataaagcttc ctccttgaaa cacaaa |

An amino acid sequence for the protein (Adapter protein CIKS) encoded by the TRAF3IP2 gene that is a regulator of T cell exhaustion/dysfunction resistance is available from the UniPROT database at accession no. 043734 or accession number NP_001157753, NP_001157755, NP_671733 or NP_679211, cDNA NM_001164281, NM 001164282, NM_001164283, NM_147200 or NM_147686 with gene location at Chr 6: 111.56-111.61 Mb.

| (SEQ ID NO: 63) | |
|---|---|
| MPPQLQETRMNRSIPVEVDESEPYPSQLLKPIPEYSPEEESEPP | |
| APNIRNMAPNSLSAPTMLHNSSGDFSQAHSTLKLANHQRPVSRQVTCLRTQVLEDSED | |
| SFCRRHPGLGKAFPSGCSAVSEPASESVVGALPAEHQFSFMEKRNQWLVSQLSAASPD | |
| TGHDSDKSDQSLPNASADSLGGSQEMVQRPQPHRNRAGLDLPTIDTGYDSQPQDVLGI | |
| RQLERPLPLTSVCYPQDLPRPLRSREFPQFEPQRYPACAQMLPPNLSPHAPWNYHYHC | |
| PGSPDHQVPYGHDYPRAAYQQVIQPALPGQPLPGASVRGLHPVQKVILNYPSPWDHEE | |
| RPAQRDCSFPGLPRHQDQPHHQPPNRAGAPGESLECPAELRPQVPQPPSPAAVPRPPS | |
| NPPARGTLKTSNLPEELRKVFITYSMDTAMEVVKFVNFLLVNGFQTAIDIFEDRIRGI | |
| DIIKWMERYLRDKTVMIIVAISPKYKQDVEGAESQLDEDEHGLHTKYIHRMMQIEFIK | |
| QGSMNFRFIPVLFPNAKKEHVPTWLQNTHVYSWPKNKKNILLRLLREEEYVAPPRGPL | |
| PTLQVVPL | |
| (SEQ ID NO: 64) | |
| 1 | gttcttctac ttacctgggc ccggagaagg tggagggaga cgagaagccg ccgagagccg |
| 61 | actaccctcc gggcccagtc tgtctgtccg tggtggatct aagcctcatc tgtatcctct |
| 121 | tgtgatggcg tgaaggaaag ccatggcaga tttccagcct ggtgatgctg tacagaacac |
| 181 | aggtggcctg cttccatgcc tcctcagctt caagaaacta gaatgaaccg aagcattcct |
| 241 | gtggaggttg atgaatcaga accataccca agtcagttgc tgaaaccaat cccagaatat |
| 301 | tccccggaag aggaatcaga accacctgct ccaaatataa ggaacatggc acccaacagc |
| 361 | ttgtctgcac ccacaatgct tcacaattcc tccggagact tttctcaagc tcactcaacc |
| 421 | ctgaaacttg caaatcacca gcggcctgta tcccggcagg tcacctgcct gcgcactcaa |
| 481 | gttctggagg acagtgaaga cagtttctgc aggagacacc caggcctggg caaagctttc |
| 541 | ccttctgggt gctctgcagt cagcgagcct gcgtctgagt ctgtggttgg agccctccct |
| 601 | gcagagcatc agttttcatt tatggaaaaa cttaatcaat ggctggtatc tcagctttca |
| 661 | gcggcttctc ctgacactgg ccatgactca gacaaatcag accaaagttt acctaatgcc |
| 721 | tcagcagact ccttgggcgg tagccaggag atggtgcaac ggccccagcc tcacaggaac |
| 781 | cgagcaggcc tggatctgcc aaccatagac acgggatatg attcccagcc ccaggatgtc |
| 841 | ctgggcatca ggcagctgga aaggcccctg ccccccacct ccgtgtgtta cccccaggac |
| 901 | ctccccagac ctctcaggtc cagggagttc cctcagtttg aacctcagag gtatccagca |
| 961 | tgtgcacaga tgctgcctcc caatctttcc ccacatgctc catggaacta tcattaccat |
| 1021 | tgtcctggaa gtcccgatca ccaggtgcca tatggccatg actaccctcg agcagcctac |
| 1081 | cagcaagtga tccagccggc tctgcctggg cagcccctgc ctggagccag tgtgagaggc |
| 1141 | ctgcaccctg tgcagaaggt tatcctgaat tatcccagcc cctgggacca cgaagagagg |
| 1201 | cccgcacaga gagactgctc ctttccgggg cttccaaggc accaggacca gccacatcac |
| 1261 | cagccaccta atagagctgg tgctcctggg gagtccttgg agtgccctgc agagctgaga |
| 1321 | ccacaggttc cccagcctcc gtccccagct gctgtgccta gaccccctag caaccctcca |
| 1381 | gccagaggaa ctctaaaaac aagcaatttg ccagaagaat tgcggaaagt ctttatcact |
| 1441 | tattcgatgg acacagctat ggaggtggtg aaattcgtga actttttgtt ggtaaatggc |
| 1501 | ttccaaactg caattgacat atttgaggat agaatccgag gcattgatat cattaaatgg |
| 1561 | atggagcgct accttaggga taagaccgtg atgataatcg tagcaatcag ccccaaatac |
| 1621 | aaacaggacg tggaaggcgc tgagtcgcag ctggacgagg atgagcatgg cttacatact |
| 1681 | aagtacattc atcgaatgat gcagattgag ttcataaaac aaggaagcat gaatttcaga |
| 1741 | ttcatccctg tgctcttccc aaatgctaag aaggagcatg tgcccacctg gcttcagaac |
| 1801 | actcatgtct acagctggcc caagaataaa aaaaacatcc tgctgcggct gctgagagag |
| 1861 | gaagagtatg tggctcctcc acgggggcct ctgcccaccc ttcaggtggt tcccttgtga |
| 1921 | caccgttcat ccccagatca ctgaggccag gccatgtttg gggccttgtt ctgacagcat |
| 1981 | tctggctgag gctggtcggt agcactcctg gctggttttt ttctgttcct ccccgagagg |
| 2041 | ccctctggcc cccaggaaac ctgttgtgca gagctcttcc ccggagacct ccacacaccc |
| 2101 | tggctttgaa gtggagtctg tgactgctct gcattctctg cttttaaaaa aaccattgca |
| 2161 | ggtgccagtg tcccatatgt tcctcctgac agtttgatgt gtccattctg ggcctctcag |
| 2221 | tgcttagcaa gtagataatg taagggatgt ggcagcaaat ggaaatgact acaaacactc |
| 2281 | tcctatcaat cacttcaggc tacttttatg agttagccag atgcttgtgt atcctcagac |
| 2341 | caaactgatt catgtacaaa taataaaatg tttactcttt tgtaagatta tgttttactt |
| 2401 | atctcaaagg agatacatat aatttataat gatatgggca gttgcttcca gggacatcaa |
| 2461 | caaagctgct tagatataat attagataaa tataacagac cactctgtat taatggatta |
| 2521 | aagccagcta gttaaacaac cctttttaac cataatcatg gaagctttat tcttgcaata |
| 2581 | aagattttta ggctgggcgc agtgactcac acctgtaatc ccagcacttt gggaagctaa |
| 2641 | ggcaggcaga tcatttgagg tcaggagttt gagaccagcc tggccaacat ggtgaaaccc |
| 2701 | catctctgct aaaattacaa aaaagttagc cgggcatggt ggtgtgcacc tgtaatccca |
| 2761 | gctactcggg aggctgaggc aggagaatca cttgaacccg ggaggcagag gttgcagtga |
| 2821 | gccgagatca tgtcactgca ctctagcttg ggagacagag cgagactccg tctcaaaaaa |
| 2881 | caaacaaaca aataaaaaca cccattttta acaaaacaac tttatatagc atacagccat |
| 2941 | gattctaaat agtatgatta tggttctcag gatctgacta cataggtaaa aatatttgca |
| 3001 | tatgtgtatg aagtgttggg ggatgtaggc tagaattgta gtctgtgttc taattttggt |
| 3061 | tctaccacca attagctgta tgacctttag caagtccttt aacttttctt agattccagg |
| 3121 | gactcattta taaaatgaca tggacaaaag catccctaat cactctaaaa gatttgaagt |
| 3181 | ctaggaccta aattctaaat actcttttga ggagtgactg agttttcatt ttcataatta |
| 3241 | tgtctctcag aggacaaatt tacattttct taacagagac attttcttct tctttttttt |
| 3301 | tgtttgagac agagtctcgc tctgtcgtcc aggctggagt gcagtgctgc aatcttggct |
| 3361 | cactgcaacc tgcgcctcct gggttcaagt gattcttctg cctcaacctc ccaagtagct |
| 3421 | agacctatag gcgcctgcca ccatgcccag ctaatttttg tatttttagt agagacaggg |
| 3481 | tttcatattg gccagactgg tctcgaactc ctgaccttgt gatccgccca cctcggcctc |
| 3541 | ccaaagtgct gggattacag gtgtgagcca ccacacccag ccaacatttt cctcttttaa |
| 3601 | aaaatatctt ctcacgcctg taatcccagc acttugggag gctgaggcag gcggatcatg |
| 3661 | aggtcaggag atcaagacca tcctggctaa cacggtgaaa ctccatctct actaaaaata |
| 3721 | caaaaaaaat agccgggcgt ggtggcaggc gcctgtagtc ccagctactg gggaggctga |
| 3781 | ggcaggaaaa tggtgtcaac ccgggaggcg gagcttgcag tgagccgaga ttgcgccact |
| 3841 | gcactccagc ctgggcaata gagtgagact ccgtctcaaa aaaaaaaaaa aaaaaaaaaa |
| 3901 | cttcaacaat accctcaggc tgataatttt ggatatctat ctgtatctat atatcttgtt |
| 3961 | tacctggtct ccagaaaaag aacacataca catatccata tataaaatat gtatacatgt |
| 4021 | atcaaatcta cgtaaactat aaaggtggga tggccttaat tatggcccaa gctactaaga |
| 4081 | caatgaagac tttttggggc tgcaagctac tgcttccctt ctttatctac tagcctctta |
| 4141 | aacaaggctc acttgtgcta caagacagtc caccgttttg tttttttttt cttttttttg |
| 4201 | agacagggtc tcactctttc ccaggctgca gtacagtgac acagtctcag ctcactgcag |
| 4261 | ctttgacctt gccgggctca ggtgaccctt acacttcagc ctcccaagta gcagggacta |
| 4321 | taggtgtgca ccaacatgct tggttaattt ttgtattttt tgtagagaca gggttttgcc |
| 4381 | atgttgtcca ggctagtctc gaattcctgg gctcaagtga ttcacctgcc ttggcctccc |
| 4441 | aaagtgctag gattacagat gggagccacc acgcccagcc cagtccagct cttatatgta |
| 4501 | gcacagggaa aggacaaata cttgtcaact ataaataaga aacattgcta atgcattgca |
| 4561 | aagaacacta gtttcattta ctttataact tagatgtcta ctgggtgaga cgaatgtctt |
| 4621 | tgttctttaa aaaataggaa aagagaagaa aaactagcat aacataagta ctcatttgta |
| 4681 | agactttctg acatgtaaca ttagttccgt agttctgaga cctggtagaa ctgactttca |
| 4741 | tatttggata acctggaaaa cacccaaaca caaacttcaa gtcttctttc tcttttttca |
| 4801 | ttatcttttt tagtctgagg tgacaccatc attaaggatt cgacacccgt ttgtaaataa |
| 4861 | aatgacatca gcaattactc tgaaatgttt ctagtttgca aagacttagc aatgtgatgt |
| 4921 | tattaaccct tcctcccttc agagacctgt cctaagctct gaaccactca ttccttccac |
| 4981 | tcttcttacc ccaggtggtt gatgagcagt ggtccctggt gttccacaaa gagtcattaa |
| 5041 | agtgttacag ctggtagcac tggtagcaaa aaaacaaacc aaaaagtaca cacagacaca |
| 5101 | cacacacaca cgcacacata cacacacaca cgcacttggc caagtgacaa aagcttggcc |
| 5161 | cctgaaattt ctatgagatc cgatgaccac caacatcaaa gcattttttt tttttttttt |
| 5221 | ttgagacgta gtctcgctct gtcacccagg ctagagtgca gtggtgcaat cacagctcac |
| 5281 | tgcaacctcc acctcccggg ttcaagcgat tctcctgcct cagcctctcg agtagctgtg |
| 5341 | actacaggca cctgccacca tgcccggcta attttttgta tttttagtag agacggggtt |
| 5401 | tcaccgtgtt agccaggatg gtcttgatct cctgacctcg tgatccatcc gcctcggcct |
| 5461 | cccaaattgc tgggattaca ggcatgagcc accacgcccg gcccatcaaa ggaattgtaa |
| 5521 | caactatttg agagcactga caataagatt aacactcggt tgatttagat gttatgctgg |
| 5581 | tcctcaggca ttcatcttta gatatttttg gggtggaagt ggggtagggc tgacttagta |
| 5641 | aaaataacct cttagcccaa aggctttatt cagacttaca ccgatttgag gggtgggttt |
| 5701 | gtggaatgca aggttaggtt cttacctaat atttgatgac taatttagaa ttttaaatgt |
| 5761 | aattttaaat tttagtgact ggtttcaaat ctattttaac ttctagattg ttcaaagagg |
| 5821 | tctcagtaca tggctacaat caaagtatta gactagctat ttctcagctc agtgctcaga |
| 5881 | aaaattatta ctgttgatac ctttttcttt gtttcctgtt aaataaatca cctctttaaa |
| 5941 | gaca |

An amino acid sequence for the protein (Adapter protein CIKS) encoded by the TMEM167B gene that is a regulator of T cell exhaustion/dysfunction resistance is available from the UniPROT database at accession no. 043734, cDNA NM_020141 with gene location at Chr 1: 109.09-109.10.

| (SEQ ID NO: 114) | |
|---|---|
| 1 | cttccagtca cctcggcccg gatcgggaag tgtcaagcgg gcgctccccc atctccgccg |
| 61 | ctattaccac tgaacccgga ccccctaccc aggtccaggg ccagccgcca tgacgaacgt |
| 121 | gtactccttg gatgggattc tggtgtttgg tttgctcttt gtttgcacct gtgcctactt |
| 181 | caagaaagta cctcgtctca aaacctggct gctatcagag aagaagggtg tttggggtgt |
| 241 | gttttacaaa gccgctgtga ttggaaccag gctgcatgct gctgtggcaa ttgcttgtgt |
| 301 | tgtaatggcc ttttacgtcc tgtttataaa atgaattcca aagcacccaa gtcatcaact |
| 361 | gccaaccaag gggacgggga tgaagaacct gttggagacc tgaacccagt gtaggagagt |
| 421 | tcagctgaaa tcatcggtcc ccaggatgac accacagcat ctgcccctgc tatatgtggg |
| 481 | gaaaactcat ggtcacgaac attatttatg cttcagggga ctacagaaag ccagcttcct |
| 541 | ttgatctatg tgtaaatcag tccttggcag agtgcatata atgtccggat aaattacacc |
| 601 | cctcggtgat aagattacat acctccttca taaaaacctg tcatcctgtt ttgttcttca |
| 661 | gctcctcatc aggatctttt caaactgagg ctcattaggg aaggaactag gctgtgttca |
| 721 | gacttctttt gaagagagag aattttcaag acttcttttc actctttgat ttggatctgg |
| 781 | caaattgggg aggggatgct gggtgggaaa cagttatgaa atgccaagaa attctttggc |
| 841 | tttagaaatt tatctttcat gtacccatcc gggaacataa aagagaggca tagtgctcat |
| 901 | tgcaaaaaga gaacagatga agtagctgtg ttatgtgctg gtatcttgag agttttgcca |
| 961 | agaaaatctg ggcctacata aaatttgaga attatctgty tgatgagacc agaaagcagt |
| 1021 | ggcttagaca agaaaaaatc tttctgttca ccagtatcct caaatggaga cttcacttga |
| 1081 | tcagatggta tatgaaaaat gaatcaacta ttgctatttc gtaaaacctt tttatatttt |
| 1141 | ctaaatttac ttagtgctaa atactgttac tcagctttaa atgccacgac taggggaaaa |
| 1201 | agaaactatt gaagaaataa ttgtttagta tatttgcagt tggggtagaa gaaagaaatc |
| 1261 | tagtatattg attcatatac tagtaaattc atctagtata agaacttgtg atgttagatt |
| 1321 | gaagttttgt catcttataa aagacaacaa acttattttc tgtttaagtc tgagtgttat |
| 1381 | ggcaattttt agttgattac ttatttttct tagccaaatt ttaattttct tcatattgca |
| 1441 | ttgctcttta gttgtctctg gaaattctat ttactttaag gacatgagaa attcaaatga |
| 1501 | gagaagttgc tgatattcat cagtgtgttt ggacagttca tagggtccac aaatcaaatg |
| 1561 | aggttgtttc ctgaagtaga agaaaacaga actttgcaat tgatactgaa gtactttgcc |
| 1621 | atggagttag taactcctga gcagaccatt ttagatggct cagcatttgg caggaagact |
| 1681 | tctccattcc ctgcttatat ctatggaagg atcagctgtt ggatgtctag aacttctcta |
| 1741 | tttaaaaaaa aagagtaggc ctaaaattaa attattataa gcaagcatag acatgggtct |
| 1801 | tccagttgaa ttgtccatta ccgtaaaact taatggtgga caagttagct gtggttgatt |
| 1861 | cctgtgtggc agtaaattgt cttctgtctg cttactccaa ataataaaag ctgctaggaa |
| 1921 | gtttagattt tgaaataggc agtttaatgc tttgagggtt tctagaaata cagaaagtca |
| 1981 | tcaagtaaac actgcatgtc taatcatctc agagttgtgg ctgttatctc ttcaggaatt |
| 2041 | ggtccacagg gtaaatttca acaattcata cgttttccat tgtcatttct gaggaccttt |
| 2101 | gagatgagag aaaggaaatc tagtggaaca ggaaagagag ttacaccttg tgggtgtgag |
| 2161 | tttgggacct gttggcagaa gggaatgtca ctccctggaa acaggttcag catgtttgca |
| 2221 | cttgttattt tgtagcttta atgatttttg ttttctaata gggcaaatgt ctctaagctt |
| 2281 | ggtgtttaga gctgcttcat attttaaact agttccattc cacagttcta gttcaaacca |
| 2341 | gtttttacag cctcctgggt gggtcgtctt gacccaaact cttgtgttgt tacattttga |
| 2401 | gaggttttca taccagaatg taccaaaaag tgcacaaggt aaaggtaatt tacagccaga |
| 2461 | aacaaatata catttgtttc atttcttgac tttccctaag cagataagca gaaacttgtt |
| 2521 | taactcatta ctttgtttga tgtgtattac atatttttga ctaaaagtta tagaatgtta |
| 2581 | ttcctctggg ggaatctttt acaggtggag gaatggggat agcagtattg cctcagattc |
| 2641 | aaactggcat caccataaac cctgtaggcc agggtggaat gaagtcagct cctttttata |
| 2701 | gttgaaatac aattttttat tcaccattgt ctgcacaaat ccttgaaaat aaaccttttt |
| 2761 | ttccctaca |

(SEQ ID NO: 65)
MTNVYSLDGILVFGLLFVCTCAYFKKVPRLKTWLLSEKKGVWGVFYKAAVIGTRLHAAVAIACVVMAFYVLFIK

An amino acid sequence for the protein (TSR2 ribosome maturation factor) encoded by the TSR2 gene that is a regulator of T cell exhaustion/dysfunction resistance is available from the UniPROT database at accession no. Q969E8 or accession number NP_001333718, NP 001333719, NP_001333720, NP_001333721 or NP_477511, cDNA NM_001346789, NM_001346790, NM_001346791, NM_001346792 or NM_058163 with gene location at Chr X: 54.44-54.45 Mb.

| (SEQ ID NO: 67) | |
|---|---|
| MAGAAEDARALFRAGVCAALEAWPALQIAVENGFGGVHSQEKAK | |
| WLGGAVEDYFMRNADLELDEVEDFLGELLTNEFDTVVEDGSLPQVSQQLQTMFHHFQR | |
| GDGAALREMASCITQRKCKVTATALKTARETDEDEDDVDSVEEMEVTATNDGAATDGV | |
| CPQPEPSDPDAQTIKEEDIVEDGWTIVRRKK | |
| (SEQ ID NO: 68) | |
| 1 | gtggactggg gccggataat ggcgggcgct gcagaagatg cgcgagctct tctccgggct |
| 61 | ggggtctgcg cggccctgga ggcctggccg gccttgcaga tcgctgtgga gaatggcttc |
| 121 | gggggtgtgc acagccagga gaaggccaag tggctggggg gtgcagtgga ggattacttc |
| 181 | atgcgcaatg ctgacttgga gctagatgag gtggaagact tccttggaga gctgttgacc |
| 241 | aacgagtttg atacagttgt ggaagacggg agtctgcccc aggtgagcca gcaactgcag |
| 301 | accatgttcc accacttcca gaggggtgat ggggctgctc tgagggagat ggcctcctgc |
| 361 | atcactcaga gaaaatgcaa ggtcacagcc actgcactta agacagctag agagactgat |
| 421 | gaggatgaag atgatgtgga cagtgtggaa gagatggagg tcacagctac gaatgatggg |
| 481 | gctgctacag atggggtctg cccccagcct gaaccctctg atccagacgc tcagactatt |
| 541 | aaggaagagg atatagtgga agatggctgg accattgtcc ggagaaaaaa atgagtgggg |
| 601 | atgattggaa atggctttgg gcccttattt gctgttctaa gagttgtctg taggggtttt |
| 661 | ttttttgagg attgcagacc tgtggactgg ttaccccatc tccaccctct cccctgttcc |
| 721 | tctgtctggc tccctccagg gcaaggaaaa cctagggtcc ttggtcttgg gggtgggggg |
| 781 | accttttcca gcatattccc ctgggccttt agttaacatc cgagtgacga agccgggttc |
| 841 | cccagtacag tgtcttttgg gtgcggtcat tgtgaggaag gggatgagtg agtgtgggtg |
| 901 | tggctggagg aggagctaga tgccggtgga cagtgggcag gtggatttgg ggaggagagg |
| 961 | atcagcagca gcagcagtac tcagtgatag ggggctggct gggtcaggaa tggtgagatg |
| 1021 | aacatgcaag tggaagagag aatagttgaa aatgtgaatg aaagtagctc tggagcctct |
| 1081 | cccttacccc accccacaca cacgcaacag ctctgtaccc ctgcccagaa cttggaacct |
| 1141 | cccaggaatg ctgccttcct tcagcccagc ctccagtcct gggttccctc aggaattcga |
| 1201 | agaccttggc taggtggtga gcttggaaga gctgggcttt gttttctgtc tcctctaacc |
| 1261 | tctttcctta ctccttatcc catcccattt gagttcaggg gtgaggctta agatcttaat |
| 1321 | aaataatacc ttactgctta atctgtggct tgacctgtga gtagtagcct tagaatcagc |
| 1381 | cttctgggag ctcagcttca gagtttacaa gggagcagtt tctgggccca ccctagcagc |
| 1441 | ctggtacttt ggacaaccaa acagatgaac gaacggagca cagcagtgct atgaaagtta |
| 1501 | ctgtaagcct gaaataaact gtatttttct taaaaaaaaa aaaacacaca aaaaaacaca |
| 1561 | aaaaactgag tatccattgt cctcccccct gtttccctgt cctgcccgct ggccctgcca |
| 1621 | tgggtctgga cgataaaata ctgggtagcc ttggggccgg gtagaagctg taagccatgg |
| 1681 | gagaggcagg agaggtctag ttgccacccc acgtggagtt gtacgtgtga agacactggc |
| 1741 | tgaggaggca gggctgggtc ggggccccag ggtcagtggc agagctggca cttgaatcca |
| 1801 | ggtctcctac ctctggtgct ttgtggggaa ctgggtggag gcaggatctg tggtagggta |
| 1861 | ttgagggatg agggagaaaa acgacctaat tcaggtagga ctggcaacgg ctcccaccct |
| 1921 | ccctggggac agggattaat aaaaattgac atcactgtag gaatatattt tttaaaagcc |
| 1981 | cacgttttgt gaaaagatga attaaaaaac ccaagaccca gcattcggga ttgaaagtgc |
| 2041 | ccgtgatggg agttcaagta ttgactgagc tgggagggaa ggggctagag cccccaatca |
| 2101 | gacatgggca actagagtgt ggggtggggg ctcccagttt gtcccaaacc ctctaggtct |
| 2161 | tgtctcgggt ctggggggat tcggggggtt cagcagtggc tcctaaagca gccaccggtg |
| 2221 | cctcctccat ctccctgtcc tcagacagtg tgggccccgg gcagaacgtg tcccctcggc |
| 2281 | ccgcccggcc aagcacagcc atccagcgtc gctgtagttc ctctgtctca gggctgaagt |
| 2341 | accagctgag gtggctctgg gtgatcttga agacatgcct tctgtcaggc cgctcccctg |
| 2401 | cctcgggcgg tcccacctcg aagccaatga ggggcaggct gcgctgggct ttcacatcct |
| 2461 | ggcgggagga gggacagagc tggggccacc ttggggctag acctttcccc cgccccagct |
| 2521 | tctaactggt tttgcatatg ctctgtcttc agtcccctac tcaggaactc ccctactcag |
| 2581 | gaaccttccg tggctccagt gttatcaaca ggaacatgtc agaactgggg ctaaaatgca |
| 2641 | ggctggttta aatacctgtc tccccccacc cccgcctctt ctcacaaaac tgctgggcta |
| 2701 | gtctctttag cataccaaat aatgttttca ggcctttgtc tatgctattg cccctatctg |
| 2761 | catacttttt tttttttttt tttttgagac agagtcttgc cctgttgccc aggctggagt |
| 2821 | gcagtggcat gaacttggct cactgcaacc tctacctccc aggttcaagc aattctcctg |
| 2881 | cctcagcttc ctgagtagct gggactacag gcacacgtca ccacgccagg ctaatttttg |
| 2941 | tatttttagt agagacgggg tttcaccatg ttggccaggc tggtcttgaa ctcctgatct |
| 3001 | caggtgagcc acctgcctcg gcctctcaga gtgctgggat tacaggcgtg agccactgcg |
| 3061 | ctcggtccca tctgcatact cttacccact ccaaattgga cctagcagtt ccccatctct |
| 3121 | actccttcca ggaagccagg cccacaactc atcctggttt tccctacata ccacaaccac |
| 3181 | tccttgtctc tagccagtct ttgtctctca agggctgggg ttctgatttc ctcttagaga |
| 3241 | taggctcacc ttatcttcca aggctcacct tatcttccaa ggccaaggag aggtcaagga |
| 3301 | ctggatctgg ctttgccagg tggctgaaag gacccgaagg agtaggatgc atacctgagg |
| 3361 | ggctccgtag atatacagca ccaagggttc attttcaggg accacgaacc atgccttgtg |
| 3421 | ccatcctttg ccacccttct ccatgtagtg caggaagctg cagatgacgc tgttctctgc |
| 3481 | agccactgag gcctgtttct gtggccagag acaccgggca tcaatgttga cagaaggcct |
| 3541 | agcctggctc ctcctagctt taatacctcc tcagtgtagg gactgctatt cctatctcag |
| 3601 | atgaagacgc tcaagctcag gtggcaggag tgatttgtcc agtgccaccc agtgaatcag |
| 3661 | ctagagctga gattcaaagc taggttcgtc tgcctcattt gggcaacggc tgctgcctct |
| 3721 | acaggccagt ggaaggagtg tgcggatctc aggcattctt tcttcagtca gagcattcat |
| 3781 | tcctcaagcc attcacacat ttaggcctca tgcttttttc tgtctttcat cacatccatt |
| 3841 | tcctacactc attgagaggt aatctagcat tgtggtttta agggggtatg tgggtggtta |
| 3901 | atgaagtctg gcttagaatc ccagctctag atgaaaatgt attgacttgg agagcaaaga |
| 3961 | gagctgatgt gagaattaaa tgaattaata tatgcaaagt gcttagaata gttcctggca |
| 4021 | cagagtaagt actcttttaa gtgttaaata aatacctaga tttgttttta aaaaaa |

An amino acid sequence for the protein (Proto-oncogene vav) encoded by the VAV1 gene 60 that is a regulator of T cell exhaustion/dysfunction resistance is available from the UniPROT database at accession no. P15498 or accession number NP_001245135, NP_001245136 or NP_005419, cDNA NM_001258206, NM_001258207 or NM_005428 with gene location at Chr 19: 6.77-6.86 Mb.

| (SEQ ID NO: 69) | |
|---|---|
| MELWRQCTHWLIQCRVLPPSHRVTWDGAQVCELAQALRDGVLLC | |
| QLLNNLLPHAINLREVNLRPQMSQFLCLKNIRTFLSTCCEKFGLKRSELFEAFDLFDV | |
| QDFGKVIYTLSALSWTPIAQNRGIMPFPTEEESVGDEDIYSGLSDQIDDTVEEDEDLY | |
| DCVENEEAEGDEIYEDLMRSEPVSMPPKMTEYDKRCCCLREIQQTEEKYTDTLGSIQQ | |
| HFLKPLQRFLKPQDIEIIFINIEDLLRVHTHFLKEMKEALGTPGAANLYQVF KYKER | |
| FLVYGRYCSQVESASKHLDRVAAAREDVQMKLEECSQRANNGRFTLRDLLMVPMQRVL | |
| KYHLLLQELVKHTQEAMEKENLRLALDAMRDLAQCVNEVKRDNETLRQITNFQLSIEN | |
| LDQSLAHYGRPKIDGELKITSVERRSKMDRYAFLLDKALLICKRRGDSYDLKDFVNLH | |
| SFQVRDDSSGDRDNKKWSHMFLLIEDQGAQGYELFFKTRELKKKWMEQFEMAISNIYP | |
| ENATANGHDFQMFSFEETTSCKACQMLLRGTFYQGYRCHRCRASAHKECLGRVPPCGR | |
| HGQDFPGTMKKDKLHRRAQDKKRNELGLPKMEVFQEYYGLPPPPGAIGPFLRLNPGDI | |
| VELTKAEAEQNWWEGRNTSTNEIGWFPCNRVKPYVHGPPQDLSVHLWYAGPMERAGAE | |
| SILANRSDGTFLVRQRVKDAAEFAISIKYNVEVKHKIMTAEGLYRITEKKAFRGLTE | |
| LVEFYQQNSLKDCFKSLDTTLQFPFKEPEKRTISRPAVGSTKYFGTAKARYDFCARDR | |
| SELSLKEGDIIKILNKKGQQGWWRGEIYGRVGWFPANYVEEDYSEYC | |
| (SEQ ID NO: 70) | |
| 1 | agcactagct gtcgctccac aggcgagcag ggcaggcgtg cgggcgggtg ggtggtggag |
| 61 | gctgcgaggg tgcacggccg gccctgggca ggcggtagcc atggagctgt ggcgccaatg |
| 121 | cacccactgg ctcatccagt gccgggtgct gccgcccagc caccgcgtga cctgggatgg |
| 181 | ggctcaggtg tgtgaactgg cccaggccct ccgggatggt gtccttctgt gtcagctgct |
| 241 | taacaacctg ctaccccatg ccatcaacct gcgtgaggtc aacctgcgcc cccagatgtc |
| 301 | ccagttcctg tgccttaaga acattagaac cttcctgtcc acctgctgtg agaagttcgg |
| 361 | cctcaagcgg agcgagctct tcgaagcctt tgacctcttc gatgtgcagg attttggcaa |
| 421 | ggtcatctac accctgtctg ctctgtcctg gaccccgatc gcccagaaca gggggatcat |
| 481 | gcccttcccc accgaggagg agagtgtagg tgatgaagac atctacagtg gcctgtccga |
| 541 | ccagatcgac gacacggtgg aggaggatga ggacctgtat gactgcgtgg agaatgagga |
| 601 | ggcggaaggc gacgagatct atgaggacct catgcgctcg gagcccgtgt ccatgccgcc |
| 661 | caagatgaca gagtatgaca agcgctgctg ctgcctgcgg gagatccagc agacggagga |
| 721 | gaagtacact gacacgctgg gctccatcca gcagcatttc ttgaagcccc tgcaacggtt |
| 781 | cctgaaacct caagacattg agatcatctt tatcaacatt gaggacctgc ttcgtgttca |
| 841 | tactcacttc ctaaaggaga tgaaggaagc cctgggcacc cctggcgcag ccaatctcta |
| 901 | ccaggtcttc atcaaataca aggagaggtt cctcgtctat ggccgctact gcagccaggt |
| 961 | ggagtcagcc agcaaacacc tggaccgtgt ggccgcagcc cgggaggacg tgcagatgaa |
| 1021 | gctggaggaa tgttctcaga gagccaacaa cgggaggttc accctgcggg acctgctgat |
| 1081 | ggtgcctatg cagcgagttc tcaaatatca cctccttctc caggagctgg tgaaacacac |
| 1141 | gcaggaggcg atggagaagg agaacctgcg gctggccctg gatgccatga gggacctggc |
| 1201 | tcagtgcgtg aacgaggtca agcgagacaa cgagacactg cgacagatca ccaatttcca |
| 1261 | gctgtccatt gagaacctgg accagtctct ggctcactat ggccggccca agatcgacgg |
| 1321 | ggaactcaag atcacctcgg tggaacggcg ctccaagatg gacaggtatg ccttcctgct |
| 1381 | cgacaaagct ctactcatct gtaagcgcag gggagactcc tatgacctca aggactttgt |
| 1441 | aaacctgcac agcttccagg ttcgggatga ctcttcagga gaccgagaca acaagaagtg |
| 1501 | gagccacatg ttcctcctga tcgaggacca aggtgcccag ggctatgagc tgttcttcaa |
| 1561 | gacaagagaa ttgaagaaga agtggatgga gcagcttgag atggccatct ccaacatcta |
| 1621 | tccggagaat gccaccgcca acgggcatga cttccagatg ttctcctttg aggagaccac |
| 1681 | atcctgcaag gcctgtcaga tgctgcttag aggtaccttc tatcagggct accgctgcca |
| 1741 | tcggtgccgg gcatctgcac acaaggagtg tctggggagg gtccctccat gtggccgaca |
| 1801 | tgggcaagat ttcccaggaa ctatgaagaa ggacaaacta catcgcaggg ctcaggacaa |
| 1861 | aaagaggaat gagctgggtc tgcccaagat ggaggtgttt caggaatact acgggcttcc |
| 1921 | tccaccccct ggagccattg gaccctttct acggctcaac cctggagaca ttgtggagct |
| 1981 | cacgaaggct gaggctgaac agaactggtg ggagggcaga aatacatcta ctaatgaaat |
| 2041 | tggctggttt ccttgtaaca gggtgaagcc ctatgtccat ggccctcctc aggacctgtc |
| 2101 | tgttcatctc tggtacgcag gccccatgga gcgggcaggg gcagagagca tcctggccaa |
| 2161 | ccgctcggac gggactttct tggtgcggca gagggtgaag gatgcagcag aatttgccat |
| 2221 | cagcattaaa tataacgtcg aggtcaagca cattaaaatc atgacagcag aaggactgta |
| 2281 | ccggatcaca gagaaaaagg ctttccgggg gcttacggag ctggtggagt tttaccagca |
| 2341 | gaactctcta aaggattgct tcaagtctct ggacaccacc ttgcagttcc ccttcaagga |
| 2401 | gcctgaaaag agaaccatca gcaggccagc agtgggaagc acaaagtatt ttggcacagc |
| 2461 | caaagcccgc tatgacttct gcgcccgaga ccgatcagag ctgtcgctca aggagggtga |
| 2521 | catcatcaag atccttaaca agaagggaca gcaaggctgg tggcgagggg agatctatgg |
| 2581 | ccgggttggc tggttccctg ccaactacgt ggaggaagat tattctgaat actgctgagc |
| 2641 | cctggtgcct tggcagagag acgagaaact ccaggctctg agcccggcgt gggcaggcag |
| 2701 | cggagccagg ggctgtgaca gctcccggcg ggtggagact ttgggatgga ctggaggagg |
| 2761 | ccagcgtcca gctggcggtg ctcccgggat gtgccctgac atggttaatt tataacaccc |
| 2821 | cgatttcctc ttgggtcccc tcaagcagac ggggctcaag ggggttacat ttaataaaag |
| 2881 | gatgaagatg ga |

An amino acid sequence for the protein (Zinc finger protein 211) encoded by the ZNF211 gene that is a regulator of T cell exhaustion/dysfunction resistance is available from the UniPROT database at accession no. Q13398, cDNA NM_006385 with gene location at Chr 19: 57.63-57.64.

| (SEQ ID NO: 115) | |
|---|---|
| 1 | agggcgggac ttgtggcgtc ttcgcagcgg tcattttggc tgccctcccg gaggtccgtt |
| 61 | ctgtctgtca gccgctttgg tacgctgcat cgggatcgaa gtgacggacc gtgaaggcgc |
| 121 | gagagtcagg tctgagggtc gggggcagag ccgcccgcga ggccggcctg gggatagcga |
| 181 | tgctcgggtt ccccccgggt cgcccgcagc tcccggtcca gctccgccca cagactcgga |
| 241 | tggcgaccgc actgagggac ccggcttcgg ttcccatagc tacagaggtg cttttcaaac |
| 301 | ttacacaggg aagtgtgacc tttgaagatg tggccgtgta cttctcctgg gaggaatggg |
| 361 | atctccttga tgaggctcag aaacacctgt acttcgatgt gatgctggag aactttgcac |
| 421 | ttacgtcctc cctgggttgt tggtgtggag tggaacatga ggaaacacct tctgaacaga |
| 481 | gaatttctgg agaaagagtg ccacagttca ggacttccaa agaaggttca tcttcccaga |
| 541 | atgccgactc ctgtgaaata tgttgcctgg tcttgagaga tattttgcac ttggctgaac |
| 601 | accaaggaac aaactgcggg cagaaactac acacatgtgg aaaacaattc tacatcagtg |
| 661 | caaatcttca acagcaccag aggcagcaca ttacagaggc acctttcaga agttatgtgg |
| 721 | acactgcctc gtttacacag agttgcatag tccatgtgtc ggagaaaccc tttacctgca |
| 781 | gggagatcag gaaagacttc ctggccaaca tgaggtttct ccatcaagac gccactcaaa |
| 841 | caggggagaa gccaaataac agtaacaagt gtgcggtggc cttttacagt ggaaaaagtc |
| 901 | atcacaactg gggaaaatgc agtaaagcct ttagccacat agacacactt gttcaggacc |
| 961 | agagaatcct cactagagaa ggactttttg agtgcagtaa atgtgggaaa gcatgtacgc |
| 1021 | gaagatgtaa cctcattcag caccagaaag tccacagtga agaaaggcct tatgaatgca |
| 1081 | atgaatgtgg aaaattcttt acctactact ccagtttcat tatacatcag agagttcata |
| 1141 | ctggagaaag gccttatgcg tgccctgaat gtgggaaatc gtttagtcag atatacagcc |
| 1201 | tcaatagcca taggaaagtt cacactggag aaaggcctta tgaatgtggg gaatgtggga |
| 1261 | aatcttttag ccaaaggtcc aacctcatgc agcatcgcag agttcacact ggagaaaggc |
| 1321 | cttatgaatg cagcgaatgt gggaaatctt ttagccaaaa ctttagcctg atctaccacc |
| 1381 | agagagttca cactggagaa agacctcatg agtgcaatga atgtggaaaa tcctttagcc |
| 1441 | gaagctccag cctcattcac caccggagac ttcacactgg agaaagaccc tatgagtgca |
| 1501 | gtaaatgtgg gaagtcattc aagcaaagct ccagcttcag ttcacatcgg aaagtccaca |
| 1561 | caggggaaag gccttatgtg tgtggggaat gtgggaaatc ctttagccat agctccaacc |
| 1621 | ttaagaacca ccagagagtt cacactggag aaagacctgt tgagtgcagt gaatgtagca |
| 1681 | aatcctttag ctgtaaatct aacctcatta aacacctgag agttcacact ggagaaaggc |
| 1741 | cttatgagtg cagtgaatgt gggaaatcct ttagccaaag ttctagcctc attcaacacc |
| 1801 | gcagagttca cacgggaaaa aggccttatc agtgcagtca atgtgggaaa tcctttggct |
| 1861 | gcaaatctgt cctcattcaa caccagagag ttcacattgg agaaaagcct tagctgtact |
| 1921 | gagaatatgc aatttccttt tagtgtaatt atactgaagg agtacacctg tgagagagac |
| 1981 | aagtacctga tttggaagcc ccaacatcta aggatataca gtgggcggat tccccttaag |
| 2041 | ttccaggtat gtgttacact ttctaacatg ccatttagaa agtgttagac tttctcacct |
| 2101 | gccatttatg gctcttgccg tttatgtcac tgacagtttc tgaggcagaa gccgtatcat |
| 2161 | gtctaccacc tgtgaggtcc acacagtgtg tatcatttac ctcctgaacc tgctcaagga |
| 2221 | agcagacctc tgcttctccc catttgctag aagaaatcat gaatagtctg agtcttcctc |
| 2281 | tctgacaagt tagggcatgg acttgaccca gctttgtgcc agagaaccca atatgagtgt |
| 2341 | tgttggcagc ttgccaagaa ggactgtctt tttcaagaca tactggtttc atgtgacacc |
| 2401 | tccatggatt tttttccagc ctctaagtca ccaacttggg aactgcttgt ctcacgttgc |
| 2461 | tttgtttttt acaataataa aagcattatt atttaagcta cctttatctt ggcatttgat |
| 2521 | tcactctttg aggtggttca aaaacagaat cgggccctgc cagctttaca gagtaaacac |
| 2581 | ctttggaggg agagggaggt gtcatatgcc tcagtgacaa cagtataatg gaaaaataca |
| 2641 | actgtctttc cagctttggc ctaatctgaa tttgaattat tgcccaaggc cttctaggaa |
| 2701 | atactgcagg atattatggt cttaattcgt ggactggatg caaggatttt ttgtgggctc |
| 2761 | aaaggacacc ctgaggaagt gggaaattgt gacaaattcc attgcttctg ggaacaagat |
| 2821 | gaattttgtt gttattcaca agggttatta catactgccc agcactgttg aggcagtcta |
| 2881 | cccccagtgc tgccaaggag ccaagtgccc caatacaatt tagtaggcta ctgtcactgt |
| 2941 | aaaatgatgg gggaaccata attggtgtgg aaacactggg ttactaggga gtgaaggaga |
| 3001 | ctgcagcaaa ctagatggaa tgtgctcctt gtaatatgtt ctagtaattg tgcaggatca |
| 3061 | gtgtgtacag acattcttag aatttccagg catgtgtatt ttttgtagct gaggtgactg |
| 3121 | tcaaaaaata ataaacaacc taaaggtttt gtgtattcca atagcctttc tgggatgtgc |
| 3181 | acctcaagaa cattgctgca taggccagga aaacaagcat agaaagaagg ggcgggggat |
| 3241 | gtccttccag gaatgtgact ttgtaaccta ccatcatcct gtgtttaaat gaattcaaat |
| 3301 | cagtttcatt gattaccagt atttgctgcc cactgaggca aggtgtccct ccaaggtcat |
| 3361 | caggaaagag agttaatgta gggttaccaa acctagtcaa aaacacatta taccatcatt |
| 3421 | ataattatta caatgaacat atcagtcacc tcggatttac ctcaggcctt tttctaagct |
| 3481 | ctccctcccc tctctggccc tcaaggcaac cattgattta taccttcatt tataataggt |
| 3541 | ttgtttgtat tttctataat atatgatttt attaataaaa tgtctttttt tccagtttca |
| 3601 | ttcatgatgc ataattattt tgagattctt caatggagct tatatgtggt ggtcattctt |
| 3661 | tttattgctg agtggtatat tttttatgga tatactattt gttatccatt tgtttataag |
| 3721 | catttaggtt ttatctagtt ttggctatta caaataaagc tgctagaaat gta |

(SEQ ID NO: 71)
MLGFPPGRPQLPVQLRPQTRMATALRDPASGSVTFEDVAVYFSWEEWDLLDEAQKHLYFDVMLENFALTSSLGCWCG
VEHEETPSEQRISGERVPQFRTSKEGSSSQNADSCEICCLVLRDILHLAEHQGTNCGQKLHTCGKQFYISANLQQHQ
RQHITEAPFRSYVDTASFTQSCIVHVSEKPFTCREIRKDFLANMRFLHQDATQTGEKPNNSNKCAVAFYSGKSHHNW
GKCSKAFSHIDTLVQDQRILTREGLFECSKCGKACTRRCNLIQHQKVHSEERPYECNECGKFFTYYSSFIIHQRVHT
GERPYACPECGKSFSQIYSLNSHRKVHTGERPYECGECGKSFSQRSNLMQHRRVHTGERPYECSECGKSFSQNFSLI
YHQRVHTGERPHECNECGKSFSRSSSLIHHRRLHTGERPYECSKCGKSFKQSSSFSSHRKVHTGERPYVCGECGKSF
SHSSNLKNHQRVHTGERPVECSECSKSFSCKSNLIKHLRVHTGERPYECSECGKSFSQSSSLIQHRRVHTGKRPYQC
SQCGKSFGCKSVLIQHQRVHIGEKP

### Genes Downregulated for T Cell Resistance

The following genes are downregulated in T cells resistant to exhaustion/dysfunction (see Table 1): CCNC, ELOB, ELOC. IGFL4, MED1, MED12, MED13, MN1, MED19, MEOX1, MOCS3, NQO2, PRDM14, SYCE1L, SYTL2, SRRD, SPIN1, UBE2E1, ARHGAP5. ARIH1, ARIH2, ATF7IP, ATF7IP2, ATXN7L3, BORCS8, CAND1, CASP3, CBFB, CDK13, CDKN2A, CFAP54, CISH, COPS7B, CPSF4L, CUL5, CUTA, DCUN1D3, DDA1, GABARAPL1, 50 KDM5C, KIAA1551, KRTAP10-9, MADD, MAPKAP1, MED23, MED24, MYB, MYO5A, NFKBIA, PAK2, PDCL, PIK3R5, PTEN. RASA2, RNASE9, RNF7. RPS6KA1. SBNO2, SND1, ST8SIA5, TNIP1, TRIM56, TSPEAR, UBAP2L, UBE2F, UBE2L3, WDFY1, ZBTB21, and ZMYND8.

Sequences and other information relating to these genes, and their encoded proteins, is available, for example from the NCBI and UniPROT databases, which are incorporated by reference.

A few examples of protein sequences encoded by some of the genes detected as regulators of T cell exhaustion/dysfunction resistance are provided.

An amino acid sequence for the protein (Cyclin-C) encoded by the CCNC gene that is a regulator of T cell exhaustion/dysfunction resistance is available from the UniPROT database at accession no. P24863 or accession number NP 001013417, NP_005181 or NP_001350466, cDNA NM_001013399, NM_005190 or NM_001363537 with gene location at Chr 6: 99.54-99.57 Mb.

| (SEQ ID NO: 73) | |
|---|---|
| MAGNFWQSSHYLQWILDKQDLLKERQKDLKFLSEEEYWKLQIFF | |
| TNVIQALGEHLKLRQQVIATATVYFKRFYARYSLKSIDPVLMAPTCVFLASKVEEFGV | |
| VSNTRLIAAATSVLKTRFSYAFPKEFPYRMNHILECEFYLLELMDCCLIVYHPYRPLL | |
| QYVQDMGQEDMLLPLAWRIVNDTYRTDLCLLYPPFMIALACLHVACVVQQKDARQWFA | |
| ELSVDMEKILEIIRVILKLYEQWKNFDERKEMATILSKMPKPKPPPNSEGEQGPNGSQ | |
| NSSYSQS | |
| (SEQ ID NO: 74) | |
| 1 | gtcgccgccg cggagcggag tcgagccgag ctgatttgat cgaggagcgc ggttaccgga |
| 61 | cgggctgggt ctatggtcgc tccgcgggcc gctccgccgg ctggtgcttt tttatcaggg |
| 121 | caagctgtgt tccatggcag ggaacttttg gcagagctcc cactatttgc aatggatttt |
| 181 | ggataaacaa gatctgttga aggagcgcca aaaggattta aagtttctct cagaggaaga |
| 241 | atattggaag ttacaaatat tttttacaaa tgttatccaa gcattaggtg aacatcttaa |
| 301 | attaagacaa caagttattg ccactgctac ggtatatttc aagagattct atgccaggta |
| 361 | ttctctgaaa agtatagatc ctgtattaat ggctcctaca tgtgtgtttt tggcatccaa |
| 421 | agtagaggaa tttggagtag tttcaaatac aagattgatt gctgctgcta cttctgtatt |
| 481 | aaaaactaga ttttcatatg cctttccaaa ggaatttcct tataggatga atcatatatt |
| 541 | agaatgtgaa ttctatctgt tagaactaat ggattgttgc ttgatagtgt atcatcctta |
| 601 | tagacctttg ctccagtatg tgcaggacat gggccaagaa gacatgttgc ttccccttgc |
| 661 | atggaggata gtgaatgata cctacagaac ggatctttgc ctactgtatc ctcctttcat |
| 721 | gatagcttta gcttgcctac atgtagcctg tgttgtacag cagaaagatg ccaggcaatg |
| 781 | gtttgctgag ctttctgtgg atatggaaaa gattttggaa ataatcaggg ttattttaaa |
| 841 | actatatgag cagtggaaga atttcgatga gagaaaagag atggcaacca ttcttagtaa |
| 901 | gatgccaaaa ccaaaaccac ctccaaacag tgaaggagag cagggtccaa atggaagtca |
| 961 | gaactctagc tacagccaat cttaaaacat tccgaagaat tccatagtgg accacttgga |
| 1021 | aataaaccat tggacagatt tcagtaatgt cttcagtgga acacaaatga aaatgaatag |
| 1081 | cttgtttctg tcaagcatat tggaaagtga ttttattttt gcaaaataag tttttcttta |
| 1141 | atatgattct agtacataat tgattaaaat ctcttgatta taaatgtttg gaaaggttct |
| 1201 | aaggggacct acagacagac atacatagac atttcaaaat taatagcttt tgattagtat |
| 1261 | aatatttctt aatttggata ataaaaattg tagcttttta ttaagccagg aaacatgaag |
| 1321 | cataatttgt ttaaaattct ctttggtcat tgagggacca aaaaaggacg taaaatttac |
| 1381 | agtcaatcta tgagggtttt tttccctcca taagcttaac tttaaaactg tatttaagga |
| 1441 | atcaaatctt acaaaatcct ggaagatttt ggtaatgatg ttgataattt cagggaaatt |
| 1501 | aatcaagtac ctatattgat ttaaaagtgt atttrattca gtagtttgag gatcttgcca |
| 1561 | tggaagatac tagcccagcc tagcagaaaa gtgcaatatg tatagcatac tttgacattt |
| 1621 | taaacatgat agtccataac cattttgaaa tgctgggcaa actacatgaa gttatttata |
| 1681 | attaattcac agctaatcag gcattttgaa agcttaattg gattcaaaaa ccataatgtt |
| 1741 | ggaatttggt aaaattttaa tgttgatttt tactgtgaaa aggtttttat aagatataca |
| 1801 | caccctagtt taatgttgtg tcttggtgtg gatttacaga tttactacag gtattctgaa |
| 1861 | ccaggaacac aatcaggttt caggccagtt tgatactggc tgtccttaat tctaatatga |
| 1921 | gagtaggaca tcatactaaa tgttatgtca gtgggactgt actgtctgtg gaacttagca |
| 1981 | aattaatcat tttcttcaga cttgaaggag agtgataaat aaaatttgga gtcataggat |
| 2041 | attgatgcac aatttaagga ttaaacattt ttaatcaatt gtggatgatg gcttattaaa |
| 2101 | tgttgacttc ctagtataaa actgcaagaa taaaagtaac ttctccagct gta |

An amino acid sequence for the protein (Elongin B) encoded by the ELOB gene that is a regulator of T cell exhaustion/dysfunction resistance is available from the UniPROT database at accession no. Q15370 or accession number NP_009039 or NP_996896, cDNA NM_207013 or NM_007108 with gene location at Chr 16: 2.77-2.78 Mb.

| (SEQ ID NO: 75) | |
|---|---|
| MDVFLMIRRHKTTIFTDAKESSTVFELKRIVEGILKRPPDEQRL | |
| YKDDQLLDDGKTLGECGFTSQTARPQAPATVGLAFRADDTFEALCIEPFSSPPELPDV | |
| MKPQDSGSSANEQAVQ | |
| (SEQ ID NO: 76) | |
| 1 | gcggctgcgc cggcgcgtcg aggggagagg cagcagccgc gatggacgtg ttcctcatga |
| 61 | tccggcgcca caagaccacc atcttcacgg acgccaagga gtccagcacg gtgttcgaac |
| 121 | tgaagcgcat cgtcgagggc atcctcaagc ggcctcctga cgagcagcgg ctgtacaagg |
| 181 | atgaccaact cttggatgat ggcaagacac tgggcgagtg tggcttcacc agtcaaacag |
| 241 | cacggccaca ggccccagcc acagtggggc tggccttccg ggcagatgac acctttgagg |
| 301 | ccctgtgcat cgagccgtt= tccagcccgc cagagctgcc cgatgtgatg aagccccagg |
| 361 | actcgggaag cagtgccaat gaacaagccg tgcagtgagg acccccaaga ggcccatttc |
| 421 | ccccaataaa agagatttgg gagtctgcct ggttgctgcc tctttttccc gcccctccct |
| 481 | gggatgggtc ccactccctg tgggctcctt ttggggcttg tgcttggcag ttcctgtgct |
| 541 | gtcctgtctc ccagatcctg agaccctggc tgagaacttg gcccagcctg ctgcttaaag |
| 601 | gcaccatggg gacctgggtt gccctgagac ccaagccatt gttagcagct agccagccac |
| 661 | accaaccacg ccagggggag gaaagggaag gactgggaga gacacaaaga ccagagccag |
| 721 | cctcagggac aagagattcc agttttaggc ctttctcctt ctaagtgccc cccaccccca |
| 781 | tagcctgcat gtccactccc agacgatggc caagagcaga aacacaagct ggagccagtg |
| 841 | tcctggtttg acagcatgt= caacgaggga accccaagac ggacccacac aggtccaccc |
| 901 | acgctggggg ctgtaatcac ggagggaagt ggctgccccc ttaacacacc tttaataaac |
| 961 | agtctacaga ccca |

An amino acid sequence for the protein (Elongin C) encoded by the ELOC gene that is a regulator of T cell exhaustion/dysfunction resistance is available from the UniPROT database at accession no. Q15369 or accession number NP_001191786, NP 001191787, NP_001191788, NP_001191789, or NP_001191790, cDNA NM 001204857, NM_001204858, NM_001204859, NM_001204860 or NM_001204861 with gene location at Chr 8: 73.94-73.97 MB.

| (SEQ ID NO: 77) | |
|---|---|
| MDGEEKTYGGCEGPDAMYVKLISSDGHEFIVKREHALTSGTIKA | |
| MLSGPGQFAENETNEVNFREIPSHVLSKVCMYFTYKVRYTNSSTEIPEFPIAPEIALE | |
| LLMAANFLDC | |
| (SEQ ID NO: 78) | |
| 1 | cagacggaag tgagcgacac actctgcgtc ctcgcctcac caggaaacta ctaaagttcc |
| 61 | tggggaagca aagtagaatt tcataagaac aaaatggatg gagaggagaa aacctatggt |
| 121 | ggctgtgaag gacctgatgc catgtatgtc aaattgatat catctgatgg ccatgaattt |
| 181 | attgtaaaaa gagaacatgc attaacatca ggcacgataa aagccatgtt gagtggccca |
| 241 | ggtcagtttg ctgagaacga aaccaatgag gtcaatttta gagagatacc ttcacatgtg |
| 301 | ctatcgaaag tatgcatgta ttttacgtac aaggttcgct acactaacag ctccaccgag |
| 361 | attcctgaat tcccaattgc acctgaaatt gcactggaac tgctgatggc tgcgaacttc |
| 421 | ttagattgtt aaataaaata aattataata aactgttaac tcttttcagt atttaatacc |
| 481 | tgtagttcag ttagtaactt tttcatatat agcatgttgc ctgtatgcag ttgaactata |
| 541 | taaagttcat tgcaaagcag attatcttgt ttttttgcat agcaatcaaa gttgaaattt |
| 601 | gtttgctaca tcaacaaatt aaggacattt tcacaaactg agaaataaac aaatatgcca |
| 661 | attcgtaggt ggttttgcct tatcctttga atgtgactta aaatcagcaa tgatgatata |
| 721 | gtaaatactg aaatttaggt gtaaatcaat acgttctaca gggaaataat gaggctaagt |
| 781 | atttttatgt ttttagtggt tttttagaaa cctaatctta tagccgccat tagcattact |
| 841 | agagttatgc aaataattgc attataaaca tgtttataac ttagccaaaa cattgatttt |
| 901 | tataactctc caagtatgag ttgaaatttc ttatgtcttt tgataaactg cagtattctt |
| 961 | taagtgtact ttgtcctttt gtttattgct acaatttaag aactttattt aaaaacaatt |
| 1021 | ttggaaggtt actaggtaca gcttttgcag aagaaactga actgtagcca gatggtcagc |
| 1081 | aagtaaacta catgacttag agttcttgtg cattatacat tttgggtaat tcaaagtact |
| 1141 | gtatttgcat tgactataaa gcgtcgtttg caatatttat tcttccttca tagtcccttg |
| 1201 | ttgtgattat acttccagt= gtttaaatgt attttaaaag actcactatt aactttattc |
| 1261 | agatcttaaa agctacacct ttttgaacta ttccagtctt tctccatcca attagaatgt |
| 1321 | tatggaataa catgtgaaga gatacagtga aaatcatatt ataatgcatt gaagatatcc |
| 1381 | tacatgaatt atcttctatt aaaatatgtc ccaataaata tacccataat cagtgggtct |
| 1441 | tctactgaaa tcaagctcat tcagcttttc tattaagcag ccaaaaaatt ttttgaaaaa |
| 1501 | atatgacagc atcattcctt atgcatgggg aaagaagcac tcgagatggg tttagtattg |
| 1561 | tctttatctc agaggtggaa caaagttgtc attatagggt ctataaactt aagtctccag |
| 1621 | cttatttgag actggcttaa tatggctgag cccggtggct catgcctgta atcccagcac |
| 1681 | tttcagagga cgaggcgggt agatcacttg aggccaggag tttgagacca gcctggccac |
| 1741 | catggcaaaa ccccatctct accaaaaatg caaaaattag ctaggccatg gtggcacacg |
| 1801 | cctgtaatcc cagctacttg tgaggctgag gcatgagaat cgcttgaacc tgggaggtgg |
| 1861 | aggtggcagt gagccgagat cgcaccactg cactccagcc tgggtgacag agcgagactg |
| 1921 | tctcaaaaaa aaaaaaaaaa aaa |

An amino acid sequence for the protein (Insulin growth factor-like family member 4) encoded by the IGFL4 gene that is a regulator of T cell exhaustion/dysfunction resistance is available from the UniPROT database at accession no. Q6B9Z1, cDNA NM_001002923.3.

| (SEQ ID NO: 79) | |
|---|---|
| MVPRISAAIFIFELLGSNSEGVTDLRLWLCQPAPRCGEWTYNPLEQCCDDGVILDLNQTRLCGSSCTFWPCFQHCCL | |
| ESLGSQNQTVVRFKVPGMKPDCKSSPITRICAQEYHPKSPVSRSDLI | |
| (SEQ ID NO: 80) | |
| 1 | ccactgctgc tccctcgtcc gctgctctga agcctaaccc atggtgccca gaatttctgc |
| 61 | tgccatcttc atttttgaac ttttgggttc aaactcagaa ggagtcacag atcttagact |
| 121 | gtggctatgc cagccagcgc ccaggtgcgg ggagtggacc tacaacccct tggagcagtg |
| 181 | ctgtgatgac ggtgtcatcc tagacttgaa ccagacccgg ctctgcggct ccagctgcac |
| 241 | cttctggccc tgcttccagc actgctgcct ggagtctttg ggctctcaga accagacagt |
| 301 | tgtgaggttc aaggtcccag gcatgaagcc agattgcaag tcctccccta tcaccaggat |
| 361 | ctgtgcccag gaataccacc caaaaagccc tgtgtcaaga tctgacctca tctagactgt |
| 421 | tctggggtag cagagtcaca gatcactcta gaatctaata cttggtaatt atactgcatt |
| 481 | gattttgttg ttgttgtgac agagtttcac tctgtccccc aggctggagt gcaaataagc |
| 541 | aacttcagca aagtctcagg atacaaaatc aatgtacaaa aatcacaagc attcttatac |
| 601 | accaacaaca gacaaacaga gagccaaatc atgagtgaac tcccattcac aattgcttca |
| 661 | cagagaataa aatacctagg aatccaactt acaagggatg tgaaggacct cttcaaggag |
| 721 | aactacaaac cactgctcaa ggaaataaaa gaggatacaa acaaatggaa gaacattcca |
| 781 | tgctcatggg taggaagaat caatatcgtg aaaatggcca tactgcccaa ggtaatttac |
| 841 | agattcaatg ccatccccat caagctacca atgactttct tcacagaatt ggaaaaaact |
| 901 | actttaaagt tcatatggaa ccaaaaaaga gccctcatcg ccaagtcaat cctaagccaa |
| 961 | aagaacaaag ctggaggcat cacactacct gacttcaaac tatactataa ggctacagta |
| 1021 | accaaaacag catggtactg gtaccaaaac agagatatag atcaatggaa cagaacagag |
| 1081 | ccctcagaaa taacgccgca tatctacaac tatctgatct ttgac |

An amino acid sequence for the protein (Mediator of RNA polymerase II transcription subunit 1) encoded by the MED1 gene that is a regulator of T cell exhaustion/dysfunction resistance is available from the UniPROT database at accession no Q15648 or accession number NP_004765. cDNA NM_004774 with gene location at Chr 17: 39.4-39.45 Mb.

| (SEQ ID NO: 81) | |
|---|---|
| MKAQGETEESEKLSKMSSLLERLHAKFNQNRPWSETIKLVRQVM | |
| EKRVVMSSGGHQHLVSCLETLQKALKVTSLPAMTDRLESIARQNGLGSHLSASGTECY | |
| ITSDMFYVEVQLDPAGQLCDVKVAHHGENPVSCPELVQQLREKNFDEFSKHLKGLVNL | |
| YNLPGDNKLKTKMYLALQSLEQDLSKMAIMYWKATNAGPLDKILHGSVGYLTPRSGGH | |
| LMNLKYYVSPSDLLDDKTASPIILHENNVSRSLGMNASVTIEGTSAVYKLPIAPLIMG | |
| SHPVDNKWTPSFSSITSANSVDLPACFFLKFPQPIPVSRAFVQKLQNCTGIPLFETQP | |
| TYAPLYELITQFELSKDPDPIPLNHNMRFYAALPGQQHCYFLNKDAPLPDGRSLQGTL | |
| VSKITFQHPGRVPLILNLIRHQVAYNTLIGSCVKRTILKEDSPGLLQFEVCPLSESRF | |
| SVSFQHPVNDSLVCVVMDVQDSTHVSCKLYKGLSDALICTDDFIAKVVQRCMSIPVTM | |
| RAIRRKAETIQADTPALSLIAETVEDMVKKNLPPASSPGYGMTTGNNPMSGTTTPTNT | |
| FPGGPITTLFNMSMSIKDRHESVGHGEDFSKVSQNPILTSLLQITGNGGSTIGSSPTP | |
| PHHTPPPVSSMAGNTKNHPMLMNLLKDNPAQDFSTLYGSSPLERQNSSSGSPRMEICS | |
| GSNKTKKKKSSRLPPEKPKHQTEDDFQRELFSMDVDSQNPIFDVNMTADTLDTPHITP | |
| APSQCSTPPTTYPQPVPHPQPSIQRMVRLSSSDSIGPDVTDILSDIAEEASKLPSTSD | |
| DCPAIGTPLRDSSSSGHSQSTLFDSDVFQTNNNENPYTDPADLIADAAGSPSSDSPTN | |
| HFFHDGVDFNPDLLNSQSQSGFGEEYFDESSQSGDNDDFKGFASQALNTLGVPMLGGD | |
| NGETKFKGNNQADTVDFSIISVAGKALAPADLMEHHSGSQGPLLTTGDLGKEKTQKRV | |
| KEGNGTSNSTLSGPGLDSKPGKRSRTPSNDGKSKDKPPKRKKADTEGKSPSHSSSNRP | |
| FTPPTSTGGSKSPGSAGRSQTPPGVATPPIPKITIQIPKGTVMVGKPSSHSQYTSSGS | |
| VSSSGSKSHHSHSSSSSSSASTSGKMKSSKSEGSSSSKLSSSMYSSQGSSGSSQSKNS | |
| SQSGGKPGSSPITKHGLSSGSSSTKMKPQGKPSSLMNPSLSKPNISPSHSRPPGGSDK | |
| LASPMKPVPGTPPSSKAKSPISSGSGGSHMSGTSSSSGMKSSSGLGSSGSLSQKTPPS | |
| SNSCTASSSSFSSSGSSMSSSQNQHGSSKGKSPSRNKKPSLTAVIDKLKHGVVTSGPG | |
| GEDPLDGQMGVSTNSSSHPMSSKHNMSGGEFQGKREKSDKDKSKVSTSGSSVDSSKKT | |
| SESKNVGSTGVAKIIISKHDGGSPSIKAKVTLQKPGESSGEGLRPQMASSKNYGSPLI | |
| SGSTPKHERGSPSHSKSPAYTPQNLDSESESGSSIAEKSYQNSPSSDDGIRPLPEYST | |
| EKHKKHKKEKKKVKDKDRDRDRDKDRDKKKSHSIKPESWSKSPISSDQSLSMTSNTIL | |
| SADRPSRLSPDFMIGEEDDDLMDVALIGN | |
| (SEQ ID NO: 82) | |
| 1 | gaagttccgt tggggaagat ggcggcggcc tcgagcaccc ttctcttctt gccgccgggg |
| 61 | acttcagatt gatccttccc gggaagagta gggactgctg gtgccctgcg tcccgggatc |
| 121 | ccgagccaac ttgtttcctc cgttagtggt ggggaagggc ttatcctttt gtggcggatc |
| 181 | tagcttctcc tcgccttcag gatgaaagct cagggggaaa ccgaggagtc agaaaagctg |
| 241 | agtaagatga gttctctcct ggaacggctc catgcaaaat ttaaccaaaa tagaccctgg |
| 301 | agtgaaacca ttaagcttgt gcgtcaagtc atggagaaga gggttgtgat gagttctgga |
| 361 | gggcatcaac atttggtcag ctgtttggag acattgcaga aggctctcaa agtaacatct |
| 421 | ttaccagcaa tgactgatcg tttggagtcc atagcaagac agaatggact gggctctcat |
| 481 | ctcagtgcca gtggcactga atgttacatc acgtcagata tgttctatgt ggaagtgcag |
| 541 | ttagatcctg caggacagct ttgtgatgta aaagtggctc accatgggga gaatcctgtg |
| 601 | agctgtccgg agcttgtaca gcagctaagg gaaaaaaatt ttgatgaatt ttctaagcac |
| 661 | cttaagggcc ttgttaatct gtataacctt ccaggggaca acaaactgaa gactaaaatg |
| 721 | tacttggctc tccaatcctt agaacaagat ctttctaaaa tcgcaattat gtactggaaa |
| 781 | gcaactaatg ctggtccctt ggataagatt cttcatggaa gtgttggcta tctcacacca |
| 841 | aggagtgggg gtcatttaat gaacctgaag tactatgtct ctccttctga cctactggat |
| 901 | gacaagactg catctcccat cattttgcat gagaataatg tttctcgatc tttgggcatg |
| 961 | aatgcatcag tgacaattga aggaacatct gctgtgtaca aactcccaat tgcaccatta |
| 1021 | attatggggt cacatccagt tgacaataaa tggacccctt ccttctcctc aatcaccagt |
| 1081 | gccaacagtg ttgatcttcc tgcctgtttc ttcttgaaat ttccccagcc aatcccagta |
| 1141 | tctagagcat ttgttcagaa actgcagaac tgcacaggaa ttccattgtt tgaaactcaa |
| 1201 | ccaacttatg cacccctgta tgaactgatc actcagtttg agctatcaaa ggaccctgac |
| 1261 | cccatacctt tgaatcacaa catgagattt tatgctgctc ttcctggtca gcagcactgc |
| 1321 | tatttcctca acaaggatgc tcctcttcca gatggccgaa gtctacaggg aacccttgtt |
| 1381 | agcaaaatca cctttcagca ccctggccga gttcctctta tcctaaatct gatcagacac |
| 1441 | caagtggcct ataacaccct cattggaagc tgtgtcaaaa gaactattct gaaagaagat |
| 1501 | tctcctgggc ttctccaatt tgaagtgtgt cctctctcag agtctcgttt cagcgtatct |
| 1561 | tttcagcacc ctgtgaatga ctccctggtg tgtgtggtaa tggatgtgca ggactcaaca |
| 1621 | catgtgagct gtaaactcta caaagggctg tcggatgcac tgatctgcac agatgacttc |
| 1681 | attgccaaag ttgttcaaag atgtatgtcc atccctgtga cgatgagggc tattcggagg |
| 1741 | aaagctgaaa ccattcaagc cgacacccca gcactgtccc tcattgcaga gacagttgaa |
| 1801 | gacatggtga aaaagaacct gcccccggct agcagcccag ggtatggcat gaccacaggc |
| 1861 | aacaacccaa tgagtggtac cactacacca accaacacct ttccgggggg tcccattacc |
| 1921 | accttgttta atatgagcat gagcatcaaa gatcggcatg agtcggtggg ccatggggag |
| 1981 | gacttcagca aggtgtctca gaacccaatt cttaccagtt tgttgcaaat cacagggaac |
| 2041 | ggggggtcta ccattggctc gagtccgacc cctcctcatc acacgccgcc acctgtctct |
| 2101 | tcgatggccg gcaacaccaa gaaccacccg atgctcatga accttcttaa agataatcct |
| 2161 | gcccaggatt tctcaaccct ttatggaagc agccctttag aaaggcagaa ctcctcttcc |
| 2221 | ggctcacccc gcatggaaat atgctcgggg agcaacaaga ccaagaaaaa gaagtcatca |
| 2281 | agattaccac ctgagaaacc aaagcaccag actgaagatg actttcagag ggagctattt |
| 2341 | tcaatggatg ttgactcaca gaaccctatc tttgatgtca acatgacagc tgacacgctg |
| 2401 | gatacgccac acatcactcc agctccaagc cagtgtagca ctcccccaac aacttaccca |
| 2461 | caaccagtac ctcaccccca acccagtatt caaaggatgg tccgactatc cagttcagac |
| 2521 | agcattggcc cagatgtaac tgacatcctt tcagacattg cagaagaagc ttctaaactt |
| 2581 | cccagcacta gtgatgattg cccagccatt ggcacccctc ttcgagattc ttcaagctct |
| 2641 | gggcattctc agagtaccct gtttgactct gatgtctttc aaactaacaa taatgaaaat |
| 2701 | ccatacactg atccagctga tcttattgca gatgctgctg gaagccccag tagtgactct |
| 2761 | cctaccaatc atttttttca tgatggagta gatttcaatc ctgatttatt gaacagccag |
| 2821 | agccaaagtg gttttggaga agaatatttt gatgaaagca gccaaagtgg ggataatgat |
| 2881 | gatttcaaag gatttgcatc tcaggcacta aatactttgg gggtgccaat gcttggaggt |
| 2941 | gataatgggg agaccaagtt taagggcaat aaccaagccg acacagttga tttcagtatt |
| 3001 | atttcagtag ccggcaaagc tttagctcct gcagatctta tggagcatca cagtggtagt |
| 3061 | cagggtcctt tactgaccac tggggactta gggaaagaaa agactcaaaa gagggtaaag |
| 3121 | gaaggcaatg gcaccagtaa tagtactctc tcggggcccg gattagacag caaaccaggg |
| 3181 | aagcgcagtc ggaccccttc taatgatggg aaaagcaaag ataagcctcc aaagcggaag |
| 3241 | aaggcagaca ctgagggaaa gtctccatct catagttctt ctaacagacc ttttacccca |
| 3301 | cctaccagta caggtggatc taaatcgcca ggcagtgcag gaagatctca gactccccca |
| 3361 | ggtgttgcca caccacccat tcccaaaatc actattcaga ttcctaaggg aacagtgatg |
| 3421 | gtgggcaagc cttcctctca cagtcagtat accagcagtg gttctgtgtc ttcctcaggc |
| 3481 | agcaaaagcc accatagcca ttcttcctcc tcttcctcat ctgcttccac ctcagggaag |
| 3541 | atgaaaagca gtaaatcaga aggttcatca agttccaagt taagtagcag tatgtattct |
| 3601 | agccaggggt cttctggatc tagccagtcc aaaaattcat cccagtctgg ggggaagcca |
| 3661 | ggctcctctc ccataaccaa gcatggactg agcagtggct ctagcagcac caagatgaaa |
| 3721 | cctcaaggaa agccatcatc acttatgaat ccttctttaa gtaaaccaaa catatcccct |
| 3781 | tctcattcaa ggccacctgg aggctctgac aagcttgcct ctccaatgaa gcctgttcct |
| 3841 | ggaactcctc catcctctaa agccaagtcc cctatcagtt caggttctgg tggttctcat |
| 3901 | atgtctggaa ctagttcaag ctctggcatg aagtcatctt cagggttagg atcctcaggc |
| 3961 | tcgttgtccc agaaaactcc cccatcatct aattcctgta cggcatcttc ctcctccttt |
| 4021 | tcctcaagtg gctcttccat gtcatcctct cagaaccagc atgggagttc taaaggaaaa |
| 4081 | tctcccagca gaaacaagaa gccgtccttg acagctgtca tagataaact gaagcatggg |
| 4141 | gttgtcacca gtggccctgg gggtgaagac ccactggacg gccagatggg ggtgagcaca |
| 4201 | aattcttcca gccatcctat gtcctccaaa cataacatgt caggaggaga gtttcagggc |
| 4261 | aagcgtgaga aaagtgataa agacaaatca aaggcttcca cctccgggag ttcagtggat |
| 4321 | tcttctaaga agacctcaga gtcaaaaaat gtggggagca caggtgtggc aaaaattatc |
| 4381 | atcagtaagc atgatggagg ctcccctagc attaaagcca aagtgacttt gcagaaacct |
| 4441 | ggggaaagta gtggagaagg gcttaggcct caaatggctt cttctaaaaa ctatggctct |
| 4501 | ccactcatca gtggttccac tccaaagcat gagcgtggct ctcccagcca tagtaagtca |
| 4561 | ccagcatata ccccccagaa tctggacagt gaaagtgagt caggctcctc catagcagag |
| 4621 | aaatcttatc agaatagtcc cagctcagac gatggtatcc gaccacttcc agaatacagc |
| 4681 | acagagaaac ataagaagca caaaaaggaa aagaagaaag taaaagacaa agatagggac |
| 4741 | cgagaccggg acaaagaccg agacaagaaa aaatctcata gcatcaagcc agagagttgg |
| 4801 | tccaaatcac ccatctcttc agaccagtcc ttgtctatga caagtaacac aatcttatct |
| 4861 | gcagacagac cctcaaggct cagcccagac tttargattg gggaggaaga tgatgatctt |
| 4921 | atggatgtgg ccctgattgg gaattaggaa ccttatttcc taaaagaaac agggccagag |
| 4981 | gaaaaaaaac tattgataag tttataggca aaccaccata aggggtgagt cagacaggtc |
| 5041 | tgatttggtt aagaatccta aatggcatgg ctttgacatc aagctgggtg aattagaaag |
| 5101 | gcatatccag accctattaa agaaaccaca gggtctgatt ctggttacca ggaagtcttc |
| 5161 | tttgttcctg tgccagaaag aaagttaaaa tacttgctta agaaagggag gggggtggga |
| 5221 | ggggtgtagg gagagggaag ggagggaaac agttatgtgg gaaatattca tatatatttt |
| 5281 | cttctccctt tttccatttt taggccatgt tttaaactca ttttagtgca tgtatatgaa |
| 5341 | gggctgggca gaaaatgaaa aagcaataca ttccttgatg catttgcatg aaggttgttc |
| 5401 | aactttgttt gaggtagttg tccgtttgag tcatgggcaa atgaaggact ttggtcattt |
| 5461 | tggacactta agtaatgttt ggtgtctgtt tcttaggagt gactggggga gggaagatta |
| 5521 | ttttagctat ttatttgtaa tattttaacc ctttatctgt ttgtttttat acagtgtttc |
| 5581 | gttctaaatc tatgaggttt agggttcaaa atgatggaag gccgaagagc aaggcttata |
| 5641 | tggtggtagg gagcttatag cttgtgctaa tactgtagca tcaagcccaa gcaaattagt |
| 5701 | cagagcccgc ctttagagtt aaatataata gaaaaaccaa aatgatattt ttattttagg |
| 5761 | agggtttaaa tagggttcag agatcatagg aatattagga gttacctctc tgtggaggta |
| 5821 | ttgacttgta atctcatttt cctttcaaaa aaaaaaaaaa aagctaaggt ggcttgttgg |
| 5881 | gatgtaaaca tgttttcaga tgcagtaagg tttagtgtag gacagccttc ctgacccagt |
| 5941 | ggcatgaaaa ccattacagg attaatagtt ctcctacttc cacaatgtgc caaaagtctg |
| 6001 | catcccagca ttttgtttgc aggagaactg atgccattcc taagagctgg actcactgtt |
| 6061 | tctcttcatc acaaggagaa ggagtccaaa ctttaatcac tccactgtat gctccctgag |
| 6121 | ataaaacagt aaaaaatccg cagccatagt tcacttaaaa caatttcaag ctcacttttg |
| 6181 | aagtaatggg ggcctggaat gctaggtgag catgaagata aacccttgct actatgtagc |
| 6241 | aacccaattg gacctttttg gagaaatagg tctgagtctg gattctgggg gacatcaata |
| 6301 | agagcccttc acataaaaat atagaaatcc aggagactgt ttcgagtgca acagaagttc |
| 6361 | tcagtatttg gagggtcctc tcaaaaattc tgcggcctta ctttgatatt gacacctgca |
| 6421 | ctgtgccatt cctgattatt ccattcagga tctgtatcag cgggatgggg catggtcccc |
| 6481 | agcacaactc ttctgggtta aaaaaaaaaa gccaggtgat tcctttgtgt gttatgtcgt |
| 6541 | aagtggagtg acttcatcat atatggaaga agatttctat attcagcttt ttctgcaggt |
| 6601 | tggagtcagc atagagttgg aaaatcagct ttggctttct ttcctgtctc atttcctcta |
| 6661 | gtgttctcct ttttattgtc atcagctctc aacaactctg ccacttttgt gtcccaaggt |
| 6721 | aataagatgt aggaaacaaa acattgtaaa gtggagcaag aaaagttatc aattaaccac |
| 6781 | atcagagtca aatgtcttgg gtgacactaa ggaggatatg ggcaggtgat accagagtgc |
| 6841 | tttatcttgt gatgttgata cagtagcagc ctctcagaca ttcagccagg ttggatttct |
| 6901 | catgagtttg tcacctagtt ttgaatccta tccccttggt ttctgcagga aaaaaaaaaa |
| 6961 | aatttatgtg gtttttaaaa tttgttctga gtggggagaa tcttaggggg aatgtactga |
| 7021 | atagtatcat gggctcagct cccccatgca gggccaacaa ataccaaaat gagtaaactg |
| 7081 | ggaagctttt ctctctttct gtcttcatcc cagatcaaag aatcccgagt taggatctgg |
| 7141 | atgaaggata agcccctgaa ttgtcgatgg gctcaccccc acactgaccc agcatctgaa |
| 7201 | cttgcttaac agggagccgg ggctaaactg cttcaccctg cctgagaacc agggagcact |
| 7261 | gcatttctcc acagggtgga ggagaagagg cagaataaac caagcctggg acacctccct |
| 7321 | cctgtctagg tgtactcatt cttctgtttc aaaagaaggc aaggacatga agtcaacttc |
| 7381 | tacctatctt ctgctgctgg tgtcttatgt attctcagtt tgacctgatt cctcttctgt |
| 7441 | cttttgactt aacattaggg gttcttggtc ataacctgct ctgatgtaca taaagatttc |
| 7501 | aggttcaata tcaatgtgtc ttaaaacaga agtattttag cgggtggggg gtggggtggt |
| 7561 | ggggacaaac aacgcaggat ataattgcca aaaccaggct tgaggttggt gactcttgaa |
| 7621 | agattttctt tcttcaggcc tagatcagaa aattaagtgc agcaatatca tgaattctca |
| 7681 | gaagcccttt cagggagcca gtgagtcata cagtatccac agttgagtca cttaaagatg |
| 7741 | tcagtatacg aaacattatt cacaatcctt gggcaatctc attttttttt ccttctcccc |
| 7801 | tcctcccctg ccccccatac atttctatcc ttgagttagt tttggagggg caggaagtac |
| 7861 | ttaacatctc agaagctaga ttgggaaaca tgctcagcta taagaactga gctttaaatt |
| 7921 | ttgagtttaa aaatgtacat caggagcagc tggggggggt ctttttttaa aaaaatcttt |
| 7981 | caaatttggt tttctgtgca tatggccgtt ttgtaaatac tttggggttt ttcatttttt |
| 8041 | tgaaagtaga tgaaatctgt tgtgggattt ttttcccgaa acattacaaa ataacctgtt |
| 8101 | tatttacatg caaataaact tctttgataa aaagtaa |

An amino acid sequence for the protein (mediator complex subunit 12) encoded by the MED12 gene that is a regulator of T cell exhaustion/dysfunction resistance is available from the UniPROT database at accession no. Q93074 or accession number NP_005111, cDNA NM_005120 with gene location at Chr X: 71.12-71.14 Mb.

An amino acid sequence for the protein encoded by the human MED12 (mediator complex subunit 12) gene that is a regulator of T cells is available as GenBank accession no. KAI4000107.1, shown below as SEQ ID NO: 83.

| (SEQ ID NO: 83) | |
|---|---|
| 1 | MAAFGILSYE HRPLKRPRLG PPDVYPQDPK QKEDELTALN VKQGFNNQPA VSGDEHGSAK |
| 61 | NVSFNPAKIS SNFSSIIAEK LRCNTLPDTG RRKPQVNQKD NFWLVTARSQ SAINTWFTDL |
| 121 | AGTKPLTQLA KKVPIFSKKE EVFGYLAKYT VPVMRAAWLI KMTCAYYAAI SETKVKKRHV |
| 181 | DPFMEWTQII TKYLWEQLQK MAEYYRPGPA GSGGCGSTIG PLPHDVEVAI RQWDYTEKLA |
| 241 | MFMFQDGMLD RHEFLTWVLE CFEKIRPGED ELLKLLLPLL LRYSGEFVQS AYLSRRLAYF |
| 301 | CTRRLALQLD GVSSHSSHVI SAQSTSTLPT TPAPQPPTSS TPSTPFSDLL MCPQHRPLVF |
| 361 | GLSCILQTIL LCCPSALVWH YSLTDSRIKT GSPLDHLPIA PSNLPMPEGN SAFTQQVRAK |
| 421 | LREIEQQIKE RGQAVEVRWS FDKCQEATAG FTIGRVLHTL EVLDSHSFER SDFSNSLDSL |
| 481 | CNRIFGLGPS KDGHEISSDD DAVVSLLCEW AVSCKRSGRH RAMVVAKLLE KRQAEIEAER |
| 541 | CGESEAADEK GSIASGSLSA PSAPIFQDVL LQFLDTQAPM LTDPRSESER VEFFNLVLLF |
| 601 | CELIRHDVFS HNMYTCTLIS RGDLAFGAPG PRPPSPFDDP ADDPEHKEAE GSSSSKLEDP |
| 661 | GLSESMDIDP SSSVLFEDME KPDFSLFSPT MPCEGKGSPS PEKPDVEKEV KPPPKEKIEG |
| 721 | TLGVLYDQPR HVQYATHFPI PQEESCSHEC NQRLVVLFGV GKQRDDARHA IKKITKDILK |
| 781 | VLNRKGTAET DQLAPIVPLN PGDLTFLGGE DGQKRRRNRP EAFPTAEDIF AKFQHLSHYD |
| 841 | QHQVTAQVSR NVLEQITSFA LGMSYHLPLV QHVQFIFDLM EYSLSISGLI DFAIQLLNEL |
| 901 | SVVEAELLLK SSDLVGSYTT SLCLCIVAVL RHYHACLILN QDQMAQVFEG LCGVVKHGMN |
| 961 | RSDGSSAERC ILAYLYDLYT SCSHLKNKFG ELFSDFCSKV KNTIYCNVEP SESNMRWAPE |
| 1021 | FMIDTLENPA AHTFTYTGLG KSLSENPANR YSFVCNALMH VCVGHHDPDR VNDIAILCAE |
| 1081 | LTGYCKSLSA EWLGVLKALC CSSNNGTCGF NDLLCNVDVS DLSFHDSLAT FVAILIARQC |
| 1141 | LLLEDLIRCA AIPSLLNAAC SEQDSEPGAR LTCRILLHLF KTPQLNPCQS DGNKPTVGIR |
| 1201 | SSCDRHLLAA SQNRIVDGAV FAVLKAVFVL GDAELKGSGF TVTGGTEELP EEEGGGGSGG |
| 1261 | RRQGGRNISV ETASLDVYAK YVLRSICQQE WVGERCLKSL CEDSNDLQDP VLSSAQAQRL |
| 1321 | MQLICYPHRL LDNEDGENPQ RQRIKRILQN LDQWTMRQSS LELQLMIKQT PNNEMNSLLE |
| 1381 | NIAKATIEVF QQSAETGSSS GSTASNMPSS SKTKPVLSSL ERSGVWLVAP LIAKLPTSVQ |
| 1441 | GHVLKAAGEE LEKGQHLGSS SRKERDRQKQ KSMSLLSQQP FLSLVLTCLK GQDEQREGLL |
| 1501 | TSLYSQVHQI VNNWRDDQYL DDCKPKQLMH EALKLRLNLV GGMFDTVQRS TQQTTEWAML |
| 1561 | LLEIIISGTV DMQSNNELFT TVLDMLSVLI NGTLAADMSS ISQGSMEENK RAYMNLAKKL |
| 1621 | QKELGERQSD SLEKVRQLLP LPKQTRDVIT CEPQGSLIDT KGNKIAGFDS IFKKEGLQVS |
| 1681 | TKQKISPWDL FEGLKPSAPL SWGWFGTVRV DRRVARGEEQ QRLLLYHTHL RPRPRAYYLE |
| 1741 | PLPLPPEDEE PPAPTLLEPE KKAPEPPKTD KPGAAPPSTE ERKKKSTKGK KRSQPATKTE |
| 1801 | DYGMGPGRSG PYGVTVPPDL LHHPNPGSIT HLNYRQGSIG LYTQNQPLPA GGPRVDPYRP |
| 1861 | VRLPMQKLPT RPTYPGVLPT TMTGVMGLEP SSYKTSVYRQ QQPAVPQGQR LRQQLQSQGM |
| 1921 | LGQSSVHQMT PSSSYGLQTS QGYTPYVSHV GLQQHTGPAG TMVPPSYSSQ PYQSTHPSTN |
| 1981 | PTLVDPTRHL QQRPSGYVHQ QAPTYGHGLT STQRFSHQTL QQTPMISTMT PMSAQGVQAG |
| 2041 | VRSTAILPEQ QQQQQQQQQQ QQQQQQQQQQ QQQQQYHIRQ QQQQQILRQQ QQQQQQQQQQ |
| 2101 | QQQQQQQQQQ QQQQHQQQQQ QQAAPPQPQP QSQPQFQRQG LQQTQQQQQT AALVRQLQQQ |
| 2161 | LSNTQPQPST NIFGRY |

A cDNA sequence encoding the protein is available from the NCBI database as accession no. NM_005120.3.

| (SEQ ID NO: 84) | |
|---|---|
| 1 | agtttccggc aatggtcgag agtttctaac gtgccccctt gttgtctctc ggccgccgtc |
| 61 | ctctcaacca ccgcccccct tttcggctcc ctctccccct tcccgttccc ccagtcagcc |
| 121 | tggccctgct ggtgcctccg gcgctacggg ctgggcaaga tggcggcctt cgggatcttg |
| 181 | agctacgaac accggcccct gaagcggccg cggctggggc ctcccgatgt ttaccctcag |
| 241 | gaccccaaac agaaggagga tgaactgacg gccttgaatg taaaacaagg tttcaataac |
| 301 | cagcctgctg tctctgggga tgagcatggc agtgccaaga acgtcagctt caatcctgcc |
| 361 | aagatcagtt ccaacttcag cagcattatt gcagagaaat tacgttgtaa tacccttcct |
| 421 | gacactggtc gcaggaagcc ccaagtgaac cagaaggata acttctggct ggtgactgca |
| 481 | cgatcccaga gtgccattaa cacttggttc actgacttgg ctggcaccaa gccactcacg |
| 541 | caactagcca aaaaggtccc cattttcagt aagaaggaag aggtgtttgg gtacttagcc |
| 601 | aaatacacag tgcctgtgat gcgggctgcc tggctcatta agatgacctg tgcctactat |
| 661 | gcagcaatct ctgagaccaa ggttaagaag agacatgttg accctttcat ggaatggact |
| 721 | cagatcatca ccaagtactt atgggagcag ttacagaaga tggctgaata ctaccggcca |
| 781 | gggcctgcag gaagtggggg ctgtggttcc acgatagggc ccttgcccca tgatgtagag |
| 841 | gtggcaatcc ggcagtggga ttacaccgag aagctggcca tgttcatgtt tcaggatgga |
| 901 | atgctggaca gacatgagtt cctgacctgg gtgcttgagt gttttgagaa gatccgccct |
| 961 | ggagaggatg aattgcttaa actgctgctg cctctgcttc tccgatactc tggggaattt |
| 1021 | gttcagtctg catacctgtc ccgccggctt gcctacttct gtacacggag actggccctg |
| 1081 | cagctggatg gtgtgagcag tcactcatct catgttatat ctgctcagtc aacaagcacg |
| 1141 | ctacccacca cccctgctcc tcagccccca actagcagca caccctcgac tccctttagt |
| 1201 | gacctgctta tgtgccctca gcaccggccc ctggcttttg gcctcagctg tatcctacag |
| 1261 | accatcctcc tgtgctgtcc tagtgccttg gtttggcact actcactgac tgatagcaga |
| 1321 | attaagaccg gctcaccact tgaccacttg cctattgccc cgtccaacct gcccatgcca |
| 1381 | gagggtaaca gtgccttcac tcagcaggtc cgtgcaaagt tgcgggagat cgagcagcag |
| 1441 | atcaaggagc ggggacaggc agttgaagtt cgctggtctt tcgataaatg ccaggaagct |
| 1501 | actgcaggct tcaccattgg acgggtactt catactttgg aagtgctgga cagccatagt |
| 1561 | tttgaacgct ctgacttcag caactctctt gactcccttt gtaaccgaat ctttggattg |
| 1621 | ggacctagca aggatgggca tgagatctcc tcagatgatg atgctgtggt gtcattgcta |
| 1681 | tgtgaatggg ctgtcagctg caagcgttct ggtcggcatc gtgctatggt ggtagccaag |
| 1741 | ctcctggaga agagacaggc ggagattgag gctgagcgtt gtggagaatc agaagccgca |
| 1801 | gatgagaagg gttccatcgc ctctggctcc ctttctgctc ccagtgctcc cattttccag |
| 1861 | gatgtcctcc tgcagtttct ggatacacag gctcccatgc tgacggaccc tcgaagtgag |
| 1921 | agtgagcggg tggaattctt taacttagta ctgctgttct gtgaactgat tcgacatgat |
| 1981 | gttttctccc acaacatgta tacttgcact ctcatctccc gaggggacct tgcctttgga |
| 2041 | gcccctggtc cccggcctcc ctctcccttt gatgatcctg ccgatgaccc agagcacaag |
| 2101 | gaggctgaag gcagcagcag cagcaagctg gaagatccag ggctctcaga atctatggac |
| 2161 | attgacccta gttccagtgt tctctttgag gacatggaga agcctgattt ctcattgttc |
| 2221 | tcccctacta tgccctgtga ggggaagggc agtccatccc ctgagaagcc agatgtcgag |
| 2281 | aaggaggtga agcccccacc caaggagaag attgaaggga cccttggggt tctttacgac |
| 2341 | cagccacgac acgtgcagta cgccacccat tttcccatcc cccaggagga gtcatgcagc |
| 2401 | catgagtgca accagcggtt ggtcgtactg tttggggtgg gaaagcagcg agatgatgcc |
| 2461 | cgccatgcca tcaagaaaat caccaaggat atcttgaagg ttctgaaccg caaagggaca |
| 2521 | gcagaaactg accagcttgc tcctattgtg cctctgaatc ctggagacct gacattctta |
| 2581 | ggtggggagg atgggcagaa gcggcgacgc aaccggcctg aagccttccc cactgctgaa |
| 2641 | gatatctttg ctaagttcca gcacctttca cattatgacc aacaccaggt cacggctcag |
| 2701 | gtctcccgga atgttctgga gcagatcacg agctttgccc ttggcatgtc ataccacttg |
| 2761 | cctctggtgc agcatgtgca gttcatcttc gacctcatgg aatattcact cagcatcagt |
| 2821 | ggcctcatcg actttgccat tcagctgctg aatgaactga gtgtagttga ggctgagctg |
| 2881 | cttctcaaat cctcggatct ggtgggcagc tacactacta gcctgtgcct gtgcatcgtg |
| 2941 | gctgtcctgc ggcactatca tgcctgcctc atccccaacc aggaccagat ggcacaggtc |
| 3001 | tttgaggggc tgtgtggcgt cgtgaagcat gggatgaacc ggtccgatgg ctcctctgca |
| 3061 | gagcgctgta tccttgctta tctctatgat ctgtacacct cctgtagcca tttaaagaac |
| 3121 | aaatttgggg agctcttcag cgacttttgc tcaaaggtga agaacaccat ctactgcaac |
| 3181 | gtggagccat cggaatcaaa tatgcgctgg gcacctgagt tcatgatcga cactctagag |
| 3241 | aaccctgcag ctcacacctt cacctacacg gggcraggca agagtcttag tgagaaccct |
| 3301 | gctaaccgct acagctttgt ctgcaatgcc cttatgcacg tctgtgtggg gcaccatgat |
| 3361 | cccgataggg tgaatgacat cgcaatcctg tgtgcagagc tgaccggcta ttgcaagtca |
| 3421 | ctgagtgcag aatggctagg agtgcttaag gcctcgtgct gctcctctaa caatggcact |
| 3481 | tgtggtttca acgatctcct ctgcaatgtt gatgtcagtg acctatcttt tcatgactcg |
| 3541 | ctggctactt ttgttgccat cctcatcgct cggcagtgtt tgctcctgga agatctgatt |
| 3601 | cgctgtgctg ccatcccttc actccttaat gctgcttgta gtgaacagga ctctgagcca |
| 3661 | ggggcccggc ttacctgccg catcctcctt caccttttca agacaccgca gctcaatcct |
| 3721 | tgccagtctg atggaaacaa gcctacagta ggaatccgct cctcctgcga ccgccacctg |
| 3781 | ctggctgcct cccagaaccg catcgtggat ggagccgtgt ttgctgttct caaggctgtg |
| 3841 | tttgtacttg gggatgcgga actgaaaggt tcaggcttca ctgtgacagg aggaacagaa |
| 3901 | gaacttccag aggaggaggg aggaggtggc agtggtggtc ggaggcaggg tggccgcaac |
| 3961 | atctctgtgg agacagccag tctggatgtc tatgccaagt acgtgctgcg cagcatctgc |
| 4021 | caacaggaat gggtaggaga acgttgcctt aagtctctgt gtgaggacag caatgacctg |
| 4081 | caagacccag tgttgagtag tgcccaggcg cagcgcctca tgcagctcat ttgctatcca |
| 4141 | catcgactgc tggacaatga ggatggggaa aacccccagc ggcagcgcat aaagcgcatt |
| 4201 | ctccagaact tggaccagtg gaccatgcgc cagtcttcct tggagctgca gctcatgatc |
| 4261 | aagcagaccc ctaacaatga gatgaactcc ctcttggaga acatcgccaa ggccacaatc |
| 4321 | gaggttttcc aacagtcagc agagacaggg tcatcttctg gaagtactgc aagcaacatg |
| 4381 | cccagcagca gcaagaccaa gcctgtgctc agctctctag agcgctctgg tgtatggctg |
| 4441 | gtggcccccc tcattgctaa actgcccacc tcagtccagg gacatgtgtt aaaggctgct |
| 4501 | ggggaagaat tggagaaggg tcagcacctg ggttcctctt cacgcaaaga acgtgatcga |
| 4561 | caaaagcaga agagcatgtc cctattgagc cagcagccct tcttatcgct ggtgctaaca |
| 4621 | tgtctgaaag ggcaggatga acaacgcgag ggactcctta cctccctcta cagccaggtg |
| 4681 | caccagattg tgaataattg gcgagatgac cagtacttag atgattgcaa accaaagcag |
| 4741 | cttatgcatg aggcactcaa actgcggctc aacctggtgg ggggcatgtt tgacacggtg |
| 4801 | cagcgcagca cccagcagac cacggagtgg gccatgctcc tcctggagat catcatcagc |
| 4861 | ggcactgtcg acatgcagtc caacaatgag ctcttcacta ctgtgttgga catgctgagc |
| 4921 | gtgctcatca atgggacatt ggctgcagac atgtctagca tctcgcaagg tagcatggag |
| 4981 | gaaaacaagc gtgcatacat gaacctggcg aagaagttgc agaaggagtt gggggagcgc |
| 5041 | cagtcagaca gtctggaaaa ggttcgccag ctgctgccac tgcccaagca gacccgagat |
| 5101 | gtcatcacgt gtgagccaca gggctccctt atcgatacca agggcaacaa gattgctggc |
| 5161 | ttcgattcca tcttcaagaa ggagggtcta caggcttcca ccaaacagaa gatctcgccc |
| 5221 | tgggatcttt ttgaggggtt gaagccgtca gcaccactct cttggggctg gtttggaaca |
| 5281 | gtccgagtgg accggcgagt ggctcgagga gaggagcagc agcggttgct gctctaccac |
| 5341 | acacacctga ggccccggcc ccgcgcctat tacctggagc cactgccact gcccccagaa |
| 5401 | gatgaggagc cgcctgctcc taccctgcta gagcctgaga aaaaggctcc agagcccccc |
| 5461 | aaaactgaca aaccgggggc tgctccaccc agtactgagg aacgcaagaa gaagtccacc |
| 5521 | aagggcaaga aacgcagcca gccagctacc aagacagagg actatggaat gggcccgggt |
| 5581 | cggagcggcc cttatggtgt gacagtgcct ccggacctcc tgcaccaccc aaaccctggt |
| 5641 | tctataacac accttaacta caggcaaggc tccataggcc tgtacaccca gaaccagcca |
| 5701 | ctacctgcag gtggccctcg tgtggaccca taccgtcctg tgcgcttacc aatgcagaag |
| 5761 | ctgcccaccc gaccaactta ccctggagtg ctgcccacaa ccatgactgg cgtcatgggt |
| 5821 | ttagaaccct cctcttataa gacctctgtg taccggcagc agcaacctgc ggtgccccaa |
| 5881 | ggacagcgcc ttcgccaaca gctccagcag agtcagggca tgttgggaca gtcatctgtc |
| 5941 | catcagatga ctcccagctc ttcctacggt ttgcagactt cccagggcta tactccttat |
| 6001 | gtttctcatg tgggattgca gcaacacaca ggccctgcag gtaccatggt gccccccagc |
| 6061 | tactccagcc agccttacca gagcacccac ccttctacca atcctactct tgtagatcct |
| 6121 | acccgccacc tgcaacagcg gcccagtggc tatgtgcacc agcaggcccc cacctatgga |
| 6181 | catggactga cctccactca aaggttttca caccagacac tgcagcagac acccatgata |
| 6241 | agtaccatga ctccaatgag tgcccagggc gtccaggcag gcgtccgttc aacagccatc |
| 6301 | ctacctgagc agcagcagca gcagcaacag cagcaacagc aacagcagca gcagcagcaa |
| 6361 | cagcaacagc agcagcagca gcagcagtac cacatccggc agcagcagca gcagcagatc |
| 6421 | ctgcggcagc agcagcaaca gcaacagcag cagcagcagc agcagcaaca gcaacagcag |
| 6481 | cagcagcaac agcaacaaca gcaacaccag cagcaacagc agcaacaggc ggctcctccc |
| 6541 | caaccccagc cccagtccca gccccagttc cagcgccagg ggcttcagca gacccagcag |
| 6601 | cagcaacaga cagcagcttc ggtccggcaa cttcaacaac agctctctaa tacccagcca |
| 6661 | cagcccagta ccaacatatt tggacgctac tgagccacct ggaggaactg cttgtgcact |
| 6721 | ggatgtggcc ccaccctttc ctcttaattc ccaatcccat tcctgggcta gcaccagtag |
| 6781 | tggttggggc cctcccctca ggctccattt ttaataagtt tttagtattt ttgttaatgt |
| 6841 | gaggcattga gctgttgggt tttgtatatt atttatatag agaccccaga gctgttgcac |
| 6901 | ccaatacaca gagcttcttt gcaaa |

An amino acid sequence for the protein (mediator complex subunit 13) encoded by the MED13 gene that is a regulator of T cell exhaustion/dysfunction resistance is available from the UniPROT database at accession no. Q9UHV7 or accession number NM_005121, cDNA NP_005112 with gene location at Chr 17: 61.94-62.07 Mb.

| (SEQ ID NO: 85) | |
|---|---|
| MSASFVPNGASLEDCHCNLFCLADLTGIKWKKYVWQGPTSAPIL | |
| FPVTEEDPILSSFSRCLKADVLGVWRRDQRPGRRELWIFWWGEDPSFADLIHHDLSEE | |
| EDGVWENGLSYECRTLLFKAVHNLLERCLMNRNFVRIGKWFVKPYEKDEKPINKSEHL | |
| SCSFTFFLHGDSNVCTSVEINQHQPVYLLSEEHITLAQQSNSPFQVILCPFGLNGTLT | |
| GQAFKMSDSATKKLIGEWKQFYPISCCLKEMSEEKQEDMDWEDDSLAAVEVLVAGVRM | |
| IYPACFVLVPQSDIPTPSPVGSTHCSSSCLGVHQVPASTRDPAMSSVTLTPPTSPEEV | |
| QTVDPQSVQKWVKFSSVSDGFNSDSTSHHGGKIPRKLANHVVDRVWQECNMNRAQNKR | |
| KYSASSGGLCEEATAAKVASWDFVEATQRTNCSCLRHKNLKSRNAGQQGQAPSLGQQQ | |
| QILPKHKTNEKQEKSEKPQKRPLTPFHHRVSVSDDVGMDADSASQRLVISAPDSQVRF | |
| SNIRTNDVAKTPQMHGTEMANSPQPPPLSPHPCDVVDEGVTKTPSTPQSQHFYQMPTP | |
| DPLVPSKPMEDRIDSLSQSFPPQYQEAVEPTVYVGTAVNLEEDEANIAWKYYKFPKKK | |
| DVEFLPPQLPSDKFKDDPVGPFGQESVTSVTELMVQCKKPLKVSDELVQQYQIKNQCL | |
| SAIASDAEQEPKIDPYAFVEGDEEFLFPDKKDRQNSEREAGKKHKVEDGTSSVTVLSH | |
| EEDAMSLFSPSIKQDAPRPTSHARPPSTSLIYDSDLAVSYTDLDNLFNSDEDELTPGS | |
| KKSANGSDDKASCKESKTGNLDPLSCISTADLHKMYPTPPSLEQHIMGFSPMNMNNKE | |
| YGSMDTTPGGTVLEGNSSSIGAQFKIEVDEGFCSPKPSEIKDFSYVYKPENCQILVGC | |
| SMFAPLKTLPSQYLPPIKLPEECIYRQSWTVGKLELLSSGPSMPFIKEGDGSNMDQEY | |
| GTAYTPQTHTSFGMPPSSAPPSNSGAGILPSPSTPRFPTPRTPRTPRTPRGAGGPASA | |
| QGSVKYENSDLYSPASTPSTCRPLNSVEPATVPSIPEAHSLYVNLILSESVMNLFKDC | |
| NFDSCCICVCNMNIKGADVGVYIPDPTQEAQYRCTCGFSAVMNRKFGNNSGLFLEDEL | |
| DIIGRNTDCGKEAEKRFEALRATSAEHVNGGLKESEKLSDDLILLLQDQCTNLFSPFG | |
| AADQDPFPKSGVISNWVRVEERDCCNDCYLALEHGRQFMDNMSGGKVDEALVKSSCLH | |
| PWSKRNDVSMQCSQDILRMLLSLQPVLQDAIQKKRTVRPWGVQGPLTWQQFHKMAGRG | |
| SYGTDESPEPLPIPTFLLGYDYDYLVLSPFALPYWERLMLEPYGSQRDIAYVVLCPEN | |
| EALLNGAKSFFRDLTAIYESCRLGQHRPVSRLLTDGIMRVGSTASKKLSEKLVAEWFS | |
| QAADGNNEAFSKLKLYAQVCRYDLGPYLASLPLDSSLLSQPNLVAPTSQSLITPPQMT | |
| NTGNANTPSATLASAASSTMTVTSGVAISTSVATANSTLTTASTSSSSSSNLNSGVSS | |
| NKLPSFPPFGSMNSNAAGSMSTQANTVQSGQLGGQQTSALQTAGISGESSSLPTQPHP | |
| DVSESTMDRDKVGIPTDGDSHAVTYPPAIVVYIIDPFTYENTDESTNSSSVWTLGLLR | |
| CFLEMVQTLPPHIKSTVSVQIIPCQYLLQPVKHEDREIYPQHLKSLAFSAFTQCRRPL | |
| PTSTNVKTLTGFGPGLAMETALRSPDRPECIRLYAPPFILAPVKDKQTELGETFGEAG | |
| QKYNVLFVGYCLSHDQRWILASCTDLYGELLETCIINIDVPNRARRKKSSARKFGLQK | |
| LWEWCLGLVQMSSLPWRVVIGRLGRIGHGELKDWSCLLSRRNLQSLSKRLKDMCRMCG | |
| ISAADSPSILSACLVAMEPQGSFVIMPDSVSTGSVFGRSTTLNMQTSQLNTPQDTSCT | |
| HILVFPTSASVQVASATYTTENLDLAFNPNNDGADGMGIFDLLDTGDDLDPDIINILP | |
| ASPTGSPVHSPGSHYPHGGDAGKGQSTDRLLSTEPHEEVPNILQQPLALGYFVSTAKA | |
| GPLPDWFWSACPQAQYQCPLFLKASLHLHVPSVQSDELLHSKHSHPLDSNQTSDVLRF | |
| VLEQYNALSWLTCDPATQDRRSCLPIHFVVLNQLYNFIMNML | |
| (SEQ ID NO: 86) | |
| 1 | ctctctctgg tcggaggcgg cggtaatggc ggatggtggg ttgtggcgcc ggcggcggct |
| 61 | gctgtgaggg acgatgagtg cctccttcgt gccgaacggg gccagcctgg aagattgtca |
| 121 | ctgtaacctc ttctgcctgg ctgacttgac aggaattaag tggaaaaaat atgtatggca |
| 181 | aggcccaact tctgccccta ttctgtttcc tgtgacagaa gaagacccca ttttgagcag |
| 241 | ttttagtcgc tgccttaagg cagatgtact tggtgtttgg cggcgagatc aaagacctgg |
| 301 | aagaagagaa ttgtggatat tttggtgggg tgaagacccc agttttgctg accttattca |
| 361 | ccatgactta tcagaagaag aagatggagt gtgggagaat ggactttcct atgaatgccg |
| 421 | tactctgctt ttcaaagcag ttcacaatct attggaacgg tgtttaatga acaggaattt |
| 481 | tgtacgtatt ggcaagtggt ttgtaaagcc ttatgaaaaa gatgaaaaac ctataaataa |
| 541 | aagtgaacac ttgtcctgct ccttcacctt tttcttgcat ggagacagca atgtttgtac |
| 601 | cagtgtggaa attaaccaac atcaacctgt ataccttctc agtgaagagc atatcaccct |
| 661 | tgctcaacag tctaatagcc catttcaagt tatcttatgc ccatttggac taaatggcac |
| 721 | tctcacagga caggcattca agatgtctga ttcagctaca aaaaaattaa ttggtgaatg |
| 781 | gaaacagttc tatcctatct catgttgctt gaaggagatg tctgaagaaa aacaggaaga |
| 841 | tatggattgg gaagatgatt ctttagctgc agtagaagtt cttgttgctg gtgtccgaat |
| 901 | gatctaccca gcatgctttg ttctagtccc tcagtcagac attcctactc ctagccctgt |
| 961 | gggatccact cactgttcat cttcttgctt gggtgtccac caagtgcctg cttccacaag |
| 1021 | agatcctgct atgtcttcgg ttacgcttac accacctacg tctcctgagg aagtccaaac |
| 1081 | agttgatcct cagtctgtcc agaagtgggt caaattttct tcagtatctg atggcttcaa |
| 1141 | ctccgatagt actagccacc atggtgggaa aatacccaga aaattagcaa atcatgtggt |
| 1201 | ggatagagtt tggcaagaat gcaatatgaa cagagcacag aacaagagga agtattctgc |
| 1261 | ttcatcaggt ggtctatgcg aagaagcgac agctgctaaa gtggcatcct gggattttgt |
| 1321 | tgaagccaca caaagaacaa attgcagttg tttgaggcac aaaaatctca agtcaagaaa |
| 1381 | tgctggacaa caaggacagg caccatcttt aggtcagcaa caacaaatac ttcctaagca |
| 1441 | caagaccaat gagaagcaag aaaagagtga aaagccacag aaacgcccct tgactccttt |
| 1501 | tcaccatcgt gtgtctgtta gtgatgatgt tggcatggac gcagattcag ccagccaaag |
| 1561 | acttgtgatc tctgctccag acagtcaagt gagattttca aatatccgaa ctaatgatgt |
| 1621 | agcaaagact cctcagatgc atggcaccga aatggcaaat tcacctcaac cacccccact |
| 1681 | tagtcctcac ccttgtgatg tggttgatga aggagtgact aaaacacctt caactcctca |
| 1741 | gagtcaacat ttttatcaaa tgccaacacc agatcccttg gttccttcta aaccaatgga |
| 1801 | agataggata gacagtttgt cccagtcttt cccacctcaa tatcaggaag ctgtagaacc |
| 1861 | tacagtatat gttggtacag cagtaaactt ggaagaagat gaagccaata tagcctggaa |
| 1921 | gtattacaag ttcccaaaga aaaaagatgt agagttttta ccacctcaac ttccaagtga |
| 1981 | taaattcaag gatgatccag ttggaccttt tggacaggaa agtgtaacat cagttacaga |
| 2041 | gttaatggtg caatgtaaga aacctttaaa agtttctgat gaattagtgc agcaatatca |
| 2101 | aattaaaaac cagtgtcttt cagcaatagc atctgatgca gaacaagaac ctaaaattga |
| 2161 | tccatatgca tttgttgaag gagatgagga attccttttt cctgataaaa aagatagaca |
| 2221 | aaatagtgag agagaagctg gaaaaaaaca caaggtagaa gatgggacat ctagtgtaac |
| 2281 | agtgttatca catgaagaag atgctatgtc attatttagt ccctctatca agcaagatgc |
| 2341 | tccacgccct actagtcatg cccgtcctcc atcaacaagt ttgatttatg actcagacct |
| 2401 | ggctgtctct tatactgacc ttgataatct cttcaattct gatgaagatg aactaacacc |
| 2461 | tggatctaaa aaatcagcaa atggatcaga tgataaagcc agctgcaagg aatcaaagac |
| 2521 | aggaaatctg gacccgttat cttgcataag cactgcagat cttcataaaa tgtatcctac |
| 2581 | accaccatca ttggaacaac atattatggg attttcccca atgaatatga ataataaaga |
| 2641 | atatggtagt atggatacaa cacctggagg aactgttcta gaaggaaata gttctagtat |
| 2701 | aggagcgcag ttcaaaattg aggttgatga gggattctgt agccccaaac cttctgaaat |
| 2761 | taaagatttt tcttatgtct ataagcctga aaattgtcaa attctagtgg gatgttccat |
| 2821 | gtttgcacct ctaaaaactc taccaagcca atatctgccc cctatcaaat tgccagaaga |
| 2881 | gtgtatttac cgtcagagtt ggactgttgg aaaattggaa ttgctttctt cagggccttc |
| 2941 | aatgccattc atcaaagagg gtgatggaag taatatggat caagaatatg gcactgctta |
| 3001 | tacacctcaa actcatactt cttttgggat gcctcctagc agtgcacctc ctagtaacag |
| 3061 | cggagcagga attcttcctt ctccatccac ccctcggttt ccaactccaa ggactccaag |
| 3121 | gactcctcgg actcctcgtg gagctggtgg acctgctagt gctcaaggtt cagtcaaata |
| 3181 | tgaaaattca gacttgtatt caccagcttc taccccatct acatgcagac cccttaattc |
| 3241 | tgttgaacct gcaactgtcc cttccatccc tgaagcacac agtctttatg taaacctcat |
| 3301 | cctttcagaa tcagttatga atttgtttaa agactgtaac tttgatagtt gttgcatctg |
| 3361 | tgtttgcaac atgaacatca agggtgccga tgttggagtt tacattccag atccaacgca |
| 3421 | ggaagcacaa tataggtgta cctgtggctt cagtgctgtc atgaacagaa aatttggaaa |
| 3481 | caattcagga ttatttcttg aagatgaact agatatcata ggacgcaata cagactgtgg |
| 3541 | caaagaagca gaaaaacgtt ttgaagctct cagggctacc tctgctgaac atgttaatgg |
| 3601 | aggactaaag gaatctgaaa aattatctga tgatttgata ttattgctac aagatcagtg |
| 3661 | cactaattta ttttcaccct ttggagcagc agaccaagat ccttttccta aaagtggtgt |
| 3721 | aattagcaat tgggtacgtg ttgaagagcg tgactgttgc aatgactgct accttgcatt |
| 3781 | agaacatggg cgtcagttca tggataacat gtcaggagga aaagttgatg aagcacttgt |
| 3841 | gaaaagttca tgcttacacc cctggtccaa aagaaacgat gtgagtatgc agtgctcaca |
| 3901 | ggatatactt cgaatgctcc tctctcttca gccagttctt caggatgcca ttcagaaaaa |
| 3961 | aagaacagta agaccttggg gtgttcaggg tcctctcact tggcaacaat ttcataaaat |
| 4021 | ggctggccga ggctcttatg gaactgatga atccccagaa ccactgccaa tccccacatt |
| 4081 | tttgttgggt tatgattatg attatctggt gctttctcca tttgctcttc cttattggga |
| 4141 | gagacttatg ctggaaccct atggatctca aagagatata gcctatgttg tactgtgtcc |
| 4201 | agaaaatgaa gccttgttaa atggagcaaa aagctttttt agagatctta ctgcaatata |
| 4261 | tgagtcctgt cgattaggtc aacatagacc tgtttctcga ctgttaacag atgggatcat |
| 4321 | gagagttgga tctactgcat caaagaaact atcagaaaag ttggtagcag aatggttttc |
| 4381 | tcaggcagct gacggtaaca atgaagcatt ttctaaactc aagctttatg cacaagtctg |
| 4441 | cagatatgac ctaggtcctt atcttgcttc cctgccattg gacagctctc tactttccca |
| 4501 | gccaaattta gttgccccta caagtcagtc tttgattact ccacctcaga tgacaaatac |
| 4561 | tggaaatgct aatactccat ctgccacctt agcatctgca gcgagcagca ctatgacagt |
| 4621 | gacttcaggt gttgccatat ctacttcagt tgccacagct aattcaactt tgaccacagc |
| 4681 | ttcaacttca tcttcatcat cctccaactt gaatagtgga gtatcatcaa ataaactacc |
| 4741 | ttcgtttcca ccctttggca gtatgaacag taatgctgca ggatccatgt ctacacaagc |
| 4801 | aaatacagtt cagagtggtc agctaggagg gcaacagaca tcagctctac agacagctgg |
| 4861 | gatttctgga gaatcatctt cacttcccac tcagccgcat cctgatgtgt ctgaaagcac |
| 4921 | gatggatcgg gataaagtgg gaatccccac agatggtgat tcacatgcag tcacgtatcc |
| 4981 | acctgcaatt gttgtttata taattgatcc ttttacatac gaaaatacag acgagagcac |
| 5041 | taactcttct agtgtgtgga cattggggct acttcgatgc tttctagaaa tggtccagac |
| 5101 | tcttcctcct catatcaaga gtactgtttc tgtacagatt attccttgtc agtacctgtt |
| 5161 | gcaacctgtg aagcatgaag atagagaaat ctatccccag catttaaaat ccctggcttt |
| 5221 | ttcggccttt acccagtgtc ggaggccact tccaacatca accaatgtga aaacattgac |
| 5281 | tggctttggt ccaggtttag ccatggaaac tgcccttaga agtcctgata gaccagagtg |
| 5341 | tattcgactt tatgcacctc cttttattct ggctccagtg aaggacaaac agacagagct |
| 5401 | aggagaaaca tttggagaag ctggacagaa atataatgtt ctttttgtgg gatactgttt |
| 5461 | atcacatgat caaaggtgga ttcttgcatc ttgcacagat ctatatggag aacttttaga |
| 5521 | aacttgtatc attaacatcg atgttccaaa tagggctcgt cggaaaaaaa gttctgctag |
| 5581 | aaaatttggt ctacagaaac tttgggagtg gtgcttagga cttgtacaaa tgagttcatt |
| 5641 | gccatggaga gttgtaattg gtcgtctagg aaggattggt catggagaat tgaaagattg |
| 5701 | gagctgtttg ctgagtcgtc gaaacttgca gtctctaagt aaaaggctca aagacatgtg |
| 5761 | tagaatgtgt ggtatatctg ctgcagactc ccctagcatt ctcagtgctt gcttggtggc |
| 5821 | aatggagccg caaggatctt ttgttattat gccagattat gtgtcaactg gttctgtatt |
| 5881 | tggaagaagc acgactctaa atatgcagac atctcagcta aataccccac aggatacatc |
| 5941 | atgtactcat atacttgtgt ttcctacttc tgcttctgtg caagtagctt cagctactta |
| 6001 | taccactgaa aatttggatt tagctttcaa tcccaacaat gatggagcag atggaatggg |
| 6061 | tatctttgat ttgttagaca caggagatga tcttgaccct gatatcatta atatccttcc |
| 6121 | tgcttctcca actggttctc ctgtacattc tccaggatct cattaccccc atggaggtga |
| 6181 | tgcgggcaag ggtcagagta ctgatcggct actatcaaca gaacctcatg aggaagtacc |
| 6241 | taatattctt cagcaaccat tggcccttgg ttactttgta tcaactgcca aagcaggtcc |
| 6301 | attacctgac tggttctggt cagcatgtcc tcaagcacaa tatcagtgtc ccctttttct |
| 6361 | taaggcctct ttgcacctcc acgtgccttc agtgcaatct gacgagctgc ttcacagtaa |
| 6421 | acactcccac ccacttgact caaatcagac ttcagatgtc ctcaggtttg ttttggaaca |
| 6481 | gtacaatgca ctctcctggc taacctgtga ccctgcaacc caggacagac gctcatgtct |
| 6541 | cccaattcat tttgtggtgc tgaatcagtt atataacttt attatgaata tgctgtgatc |
| 6601 | ttcatttgat ggaactgtgc aagaaaagaa caaggaaaaa tggatgtttc gctgcaggat |
| 6661 | taagttacaa ttatcttctc agtgaaggtc atttgtgatg gggtctaatt cttattactt |
| 6721 | caacaaatat tgttttgact tggggggagg ggctataacc ctgctatttt tcattgactc |
| 6781 | tattgaactc tttaggatga tgactgatca tacaaaacgt attataacat tttcgtagca |
| 6841 | aaattaacct tttttttttc cagtcacagt atttgtgaaa agtaatgagc catagtaccc |
| 6901 | agtcatgtta aatgaatatt aaaagcatgg agaggaaaca tgaggaacaa tgaatttcaa |
| 6961 | catatggctt cagaacatga agatgttctt gtatggatta tagtatctag tattcaaaaa |
| /021 | tgcctgcatc tcttctctta tttattgtaa gtttttaaat gtataaattg tcttatattt |
| 7081 | cttaacctct tttataaaaa ttttcctaga aggtttatac tgccttcttg ctttaaagca |
| 7141 | attggtctaa aatatatgta atcgtcttaa ttaaaaagtt gcagtagggt tgcttttaga |
| 7201 | gtattatttt tttgtaaggg ggtgggtggg acagtaaatt tgtattgtct cgatgtacag |
| 7261 | tttaacgggg atagaggggg aataatgtcc ataccattgt gtgtggagga tttacagcta |
| 7321 | agctgtagtt gcagagtaca tgtacagtaa tgaagttcac tgtgtttata aattgaaaag |
| 7381 | gtaccaggtc ttacagcatt ttatatatca catctttaca gaataacatg atggcaatat |
| 7441 | acaagtggta ttgttaggtg gtttaactta gaataaaatg agaattcttc agttatattt |
| 7501 | tgtactatgg tttagggcta tgactaatat ttcaggccat ttccggtgaa agaaacttag |
| 7561 | ttttacaaga aaaaccattt gctactgaat gcttaaacta attttagtgt ttaatgttac |
| 7621 | atgcttaaat ttttttcagt tttaacagtg gcatatttag gcatggaaat attattatga |
| 7681 | aatttatttt caggatctgc tataaggttg aaatttagcc cagctctagg cattttacaa |
| 7741 | attatttttc aagcagtcat tcttgattgt ttgacttttt tttttaaatt aaagattggg |
| 7801 | aatgtatgtg agagtatgca tatgtatggg tgtgtgtgtg tgcgcgcaat caaactgtgg |
| 7861 | tgtaaataga ttctcagtga attctggtat tcagactcta ttccactagt gaaagaacca |
| 7921 | ttttttaaac ttcccttgcc ttttttattt atttaatttt cttggtttgg agatgtcagt |
| 7981 | cccaaacacc agagtctgta cttttctata acacagctca gattaaggta gggcatatgc |
| 8041 | caaggaggtt ctcacctccc taaagaaggg acttgaattt tagggacttt aattcacccc |
| 8101 | tccttcaata caactttccc ccttcttgtt tgcacatgcc aagataactg cttttatgca |
| 8161 | ggctgtaccc ccttgaaaaa tcctttctac agtgctgctc acaaaagagc ccaagttcgc |
| 8221 | ctcctacctg cattgctgac ttgaattcac agtcgccgag tctacctagc tttcttggaa |
| 8281 | gcagtctagc aaaatttcta tttgtacgtt cactaattat ctacaaggac aaaatcagtt |
| 8341 | gtatttacaa aactctactt cagtgtttgt tttagttttt ttttttactg aaacttgttt |
| 8401 | ttgtgaatac tctgtgctta gaattaaata tcactttctt atgaacaaca taacttcttc |
| 8461 | agattgtgta tatgaaaaca ttagcaagtc ttgttttttc tatgaagcaa acacaattgg |
| 8521 | tgacaaaggt tgtcaatcat ttcttcaaaa ttataatgca gttctaatgg tcagcatatt |
| 8581 | ttgatattaa atttaaagat cacctctctg catttgtttt taaattatgc taatacacca |
| 8641 | cacattatgt tggtatgttt tgttctgtac tttccttaaa aaaaaaaaaa aaacttgtct |
| 8701 | gagatttgaa ggaaaatgtg cttatttgga atttccataa aaagagtatc ctttttatac |
| 8761 | acttaatagt gactttacaa aataaaagtt atattctcag ttgtttaaaa tcactaacct |
| 8821 | atgataacca cacctcaatt tgaaagtaga tttaaaatta ttccctgaca ggttatttaa |
| 8881 | tatggagcca taaggaggga acccagtaca caattatttt ttatttggga atcagggaat |
| 8941 | agttcccaaa tatacaggat ttattgataa gatttttttt cttcccttca tatatccatt |
| 9001 | caaactcaat ggaaagttat taaataacca ttagaaaagc tcagtagact tatttgagaa |
| 9061 | attaagcctt gtgcaggatg atggatttga cttactaatg tactgtcaca gacaaatatg |
| 9121 | ggtagttttg tttaaatagg taagcaaaat attatacttt atagcagtgg attaccaaca |
| 9181 | ccttgacttc tttgttacag tgctaacatc tttttttttg tgcaggtatc catgattatt |
| 9241 | aagcagggtg gaagttcagt attttgtcat ttaaaaagat tagttatata atgtctgctt |
| 9301 | ccagccagtg agaaacatcc agccatacct ttcttatgca agccattgag ttatcaggac |
| 9361 | tgtgaattaa cactgtatga ataaatttct gtacacctta ttgtttggcc agaaggccac |
| 9421 | caagtgtact tatatgtaat ccttaaattt taaagtagct gtaattttta aatatttcta |
| 9481 | aacttttctt aaaccactaa aattaagctc ttactactta gtcaactatc ctcagctgta |
| 9541 | ttcgtactca attgtcagta tggcacagat tactgtatta aaatattctc ctttcgtctt |
| 9601 | catatttacc ttctgaggta attttttaac ttaatgtgtt actacaaaga tttgcagatc |
| 9661 | tttaatcaag cactatgtta atactgtaat atcagaatac tatgttgcat tatttaaaat |
| 9721 | gttcaaattg aatagattaa aaagttttta aatgctattg catcatataa tttgctattc |
| 9781 | atccactatg gcattgcata tcaatcagtt aatacactta atgttgcatg agtgatattt |
| 9841 | tggtctgggt ttcctcttaa gattttagtt tgtctgaatt aaggaaaaat gtttttaata |
| 9901 | tacattctta ttttgtccca cccctccaga aataagctgg aaatcttaac tttttggggg |
| 9961 | gtcttttttg gtgttttaat gggcccagaa ctgtggttta aatttttatg tatgtatttt |
| 10021 | cttttttgtg gagtataaat ttaaaaactg gatttgggac ctaaaatact cctcaggttg |
| 10081 | atgtattcat gaagttttaa aacatcttta gttttcaaag taaactggat atgtggacct |
| 10141 | taaagttatt gagtttaagc tacaaattgt aacgtcatta ctggacatgt cagcatcaac |
| 10201 | cctctcaaaa tagcttggtc actttatgaa ggggcgtttt aaagttgttg tttagcagtg |
| 10261 | acatttaata tggtccaatt gcttttcttt ttaacgtgac aaaaagagaa taaggaacaa |
| 10321 | acactattgc tgccgaatgc cataacactg agttgtacaa attgtgattg aggaaatgaa |
| 10381 | aaggtttata ctttttaaaa aaaaaaaaac aaaaacaaaa aacaaaactt caaatggaat |
| 10441 | aaattattca tgaagccttc a |

An amino acid sequence for the protein (meningioma (disrupted in balanced translocation) 1) encoded by the MN1 gene that is a regulator of T cell exhaustion/dysfunction resistance is available from the UniPROT database at accession no. Q10571 or accession number NP_002421, cDNA NM_002430 with gene location at Chr 22: 27.75-27.8 Mb.

| (SEQ ID NO: 87) | |
|---|---|
| MFGLDQFEPQVNSRNAGQGERNENETGLSMNTHFKAPAFHTGGP | |
| PGPVDPAMSALGEPPILGMNMEPYGFHARGHSELHAGGLQAQPVHGFFGGQQPHHGHP | |
| GSHHPHQHHPHFGGNFGGPDPGASCLHGGRLLGYGGAAGGLGSQPPFAEGYEHMAESQ | |
| GPESFGPQRPGNLPDFHSSGASSHAVPAPCLPLDQSPNRAASFHGLPSSSGSDSHSLE | |
| PRRVTNQGAVDSLEYNYPGEAPSGHFDMFSPSDSEGQLPHYAAGRQVPGGAFPGASAM | |
| PRAAGMVGLSKMHAQPPQQQPQQQQQPQQQQQQHGVFFERFSGARKMPVGLEPSVGSR | |
| HPLMQPPQQAPPPPQQQPPQQPPQQQPPPPPGLLVRQNSCPPALPRPQQGEAGTPSGG | |
| LQDGGPMLPSQHAQFEYPIHRLENRSMHPYSEPVFSMQHPPPQQAPNQRLQHFDAPPY | |
| MNVAKRPRFDFPGSAGVDRCASWNGSMHNGALDNHLSPSAYPGLPGEFTPPVPDSFPS | |
| GPPLQHPAPDHQSLQQQQQQQQQQQQQQQQQQQQQQQQQQQQRQNAALMIKQMASRNQ | |
| QQRLRQPNLAQLGHPGDVGQGGLVHGGPVGGLAQPNFEREGGSTGAGRLGTFEQQAPH | |
| LAQESAWFSGPHPPPGDLLPRRMGGSGLPADCGPHDPSLAPPPPPGGSGVLFRGPLQE | |
| PMRMPGEGHVPALPSPGLQFGGSLGGLGQLQSPGAGVGLPSAASERRPPPPDFATSAL | |
| GGQPGFPFGAAGRQSTPHSGPGVNSPPSAGGGGGSSGGGGGGGAYPPQPDFQPSQRTS | |
| ASKLGALSLGSFNKPSSKDNLFGQSCLAALSTACQNMIASLGAPNLNVTFNKKNPPEG | |
| KRKLSQNETDGAAVAGNPGSDYFPGGTAPGAPGPGGPSGTSSSGSKASGPPNPPAQGD | |
| GTSLSPNYTLESTSGNDGKPVSGGGGRGRGRRKRDSGHVSPGTFFDKYSAAPDSGGAP | |
| GVSPGQQQASGAAVGGSSAGETRGAPTPHEKALTSPSWGKGAELLLGDQPDLIGSLDG | |
| GAKSDSSSPNVGEFASDEVSTSYANEDEVSSSSDNPQALVKASRSPLVTGSPKLPPRG | |
| VGAGEHGPKAPPPALGLGIMSNSTSTPDSYGGGGGPGHPGTPGLEQVRTPTSSSGAPP | |
| PDEIHPLEILQAQIQLQRQQFSISEDQPLGLKGGKKGECAVGASGAQNGDSELGSCCS | |
| EAVKSAMSTIDLDSLMAEHSAAWYMPADKALVDSADDDKTLAPWEKAKPQNPNSKEAH | |
| DLPANKASASQPGSHLQCLSVHCTDDVGDAKARASVPTWRSLHSDISNRFGTFVAALT | |
| (SEQ ID NO: 88) | |
| 1 | gttgctgctt ctcccggcgc tgggcgagag gcagacacag agaggggagc cgagaccctc |
| 61 | ggaacgcccc acgcagagcc cccccgctag tcgggagagg ggtgcgcaga ccccccagag |
| 121 | ctgcccccgc gcccccctcc ccccattccg ggctctgaga gcgcagcggg aacgccttag |
| 181 | cctgctcgcc cggcgtggcc ccccgacccg cgtacgcccc cctccctcag gtccgagagg |
| 241 | gactcgcgaa gcacctcagc gcggaggcag ggaaccggag ccttggagcg acccaacccc |
| 301 | tcgtctcgct gccctcccgc gcctgcaacg gtgcgcggag actccggcga actcagacac |
| 361 | ccaacggcgg agaacagaag cggcaggcgg cggacgtggc cgcgaagctg cgcgccgaac |
| 421 | gcagcgcacc cgctgccgag cagaggagcc gcgcctttcc ccgaccctcg gctccagccc |
| 481 | ccggcgcctg ccgcctcgca gcccctctgc gtcctcggct cgggggccgg caccggcgat |
| 541 | gccgagcgga gcctccagtc ctccgacccg ctgaagaagc agcagccgct cgcccggagc |
| 601 | ctacggggat tgtgcgagcg gatcgtgctc ggtggaggct cgggctgcgg ggcgcgggga |
| 661 | ctccgggggg cggggggagg gaccgctctg tcggtgcccg gcgccagccg cggctttgaa |
| 721 | gggtctccct cccctgccct tagcagctct gccacggact ccgggaggct gcgggcggcg |
| 781 | tcctgagggc tccccagcag acccaatcgg acttgagaag gtgatcgctc tgctctccca |
| 841 | acccccttcc ctccccattc cccccactta actttttgtc tccgttcatc cgcggcttcg |
| 901 | tcccctcccc gcagacccac ccgcggctgt gacaaccgcc cggggcatgg gccccccaac |
| 961 | acggctccta gaggccccgc ggcctcgcaa gatgagagag gccctccccg ggcagaatcg |
| 1021 | gagcttcagg agaggagcta ataccccgcc ccccgtccct cacatcaggc ggggtggagg |
| 1081 | tgcgcgctga gcccccgcgg tgctgagcgt cccggagcgc ccaatcctgg gctggaacga |
| 1141 | gtagctggcc ggaggcgcgc cgcggagagc cggctgtcat gccctattga tccccctctg |
| 1201 | ccccccgcca agtatgtttg ggctggacca attcgagccc caggtcaaca gcaggaacgc |
| 1261 | tggccagggc gagaggaact ttaacgagac cggactgagc atgaacaccc actttaaggc |
| 1321 | cccggctttc cacactgggg ggccccctgg ccctgtggat cctgctatga gcgcgctggg |
| 1381 | cgaacccccg atcttgggca tgaacatgga gccctacggc ttccacgcgc gcggccactc |
| 1441 | ggagttgcac gcaggggggc tgcaagcgca gcctgtgcac ggcttctttg gcggccagca |
| 1501 | gcctcaccac ggccacccgg gaagtcatca tccccaccag catcaccccc actttggggg |
| 1561 | caacttcggt ggcccggacc ccggggcctc gtgcctgcac gggggtcgcc tgctcggcta |
| 1621 | cggcggcgca gccggaggcc tgggcagcca gccgcccttc gccgagggct atgagcacat |
| 1681 | ggcggagagc caggggcctg agagcttcgg cccgcagcga ccggggaacc tcccggactt |
| 1741 | ccacagttca ggtgcctcca gccacgccgt gccggcccca tgcctgccgc tggaccagag |
| 1801 | ccctaaccga gccgcctcc: tccacggcct gccgtcctcc agcggctccg attcccacag |
| 1861 | tctggagcca cggagggtga cgaaccaagg agccgtcgac tcgctggaat acaattaccc |
| 1921 | gggcgaggcg ccctcgggac attttgacat gttttcgccc tctgactccg aagggcagct |
| 1981 | gcctcattat gcagcgggtc gccaggttcc tgggggcgct ttcccgggcg cctcggccat |
| 2041 | gcccagagct gcgggcatgg tgggcttgtc caaaatgcac gcccagccac cgcagcagca |
| 2101 | gccccagcag cagcagcagc cccagcagca gcagcagcag catggtgtgt tctttgagag |
| 2161 | gttcagtggg gccagaaaga tgcctgtggg tctggagccc tragtgggct ccaggcaccc |
| 2221 | gttaatgcag cctccccagc aggccccgcc accccctcag cagcagcccc cgcagcagcc |
| 2281 | gccacagcag cagccgccgc cgccacccgg gcttctagtc cgacaaaatt cgtgcccgcc |
| 2341 | tgcgctccct cggccccagc agggcgaggc gggcacgccc agcggcggcc tgcaggacgg |
| 2401 | aggccccatg ctgcccagcc agcacgcgca attcgagtat cccatccacc ggctggagaa |
| 2461 | ccggagcatg cacccttatt ccgagcctgt tttcagcatg cagcatcctc ctccgcagca |
| 2521 | ggcgcccaac cagcggctgc agcatttcga cgcgcccccc tacatgaacg tggccaagag |
| 2581 | gccgcgcttc gactttccgg gcagcgcggg agtggaccgc tgcgcttcgt ggaacggcag |
| 2641 | catgcacaac ggcgctctgg ataatcacct ctccccttcc gcctacccag gcctacccgg |
| 2701 | cgagttcaca ccgcctgtgc ccgacagctt cccttcgggg ccgcccctgc agcatccggc |
| 2761 | cccggaccac cagtccctgc aacagcagca gcagcagcag cagcagcagc aacagcagca |
| 2821 | gcagcagcag caacagcaac agcaacagca gcagcagcag cagcgccaaa acgcggccct |
| 2881 | catgattaag cagatggcgt cgcggaatca gcagcagcgg ctgcgccagc ccaacctggc |
| 2941 | tcagctaggc caccccgggg acgtgggcca gggcggcctg gtgcatggcg gcccggtggg |
| 3001 | cggcttggcc cagccgaact ttgagcgcga aggcggcagc acgggcgccg ggcgtctggg |
| 3061 | caccttcgag cagcaggcgc cgcacttggc gcaagagagc gcgtggttct caggtccgca |
| 3121 | tccgccgccc ggagacctgc tgccccgtag gatgggcggc tcgggtctgc ccgctgactg |
| 3181 | tggcccgcac gaccccagcc tggcgccccc tcctccgcct ggtggctcgg gggtgctgtt |
| 3241 | ccggggccct ctgcaggagc cgatgaggat gcccggagag ggccacgtgc ccgcgctgcc |
| 3301 | ttcaccgggc ctgcagttcg ggggcagtct gggaggcctg ggtcagctgc agtcgcccgg |
| 3361 | ggcgggcgtg gggctcccca gcgctgcttc ggagcgccgg cccccgccgc cggactttgc |
| 3421 | tacgtctgcg ctcgggggcc agccgggctt tccgtttggt gcagccggcc ggcagtccac |
| 3481 | gccgcacagc ggtccaggcg tgaactcgcc ccccagcgcg ggagggggcg gtggcagctc |
| 3541 | tggtggcggc ggtggcgggg gtgcctaccc gccgcagcct gatttccagc ccagccagcg |
| 3601 | cacctcggcc agtaaattgg gcgcgctctc gctgggctcc ttcaacaagc ccagctccaa |
| 3661 | ggacaacctg ttcggccaga gctgcctggc tgcgctctcc accgcttgcc agaacatgat |
| 3721 | cgccagcctc ggggccccca acctcaacgt gaccttcaac aagaagaacc cgccagaggg |
| 3781 | caagaggaaa ctgagccaga acgagaccga cggcgcggca gtggccggca acccgggctc |
| 3841 | ggattacttc ccaggaggga ctgctcctgg ggccccagga cccggaggcc cgtccgggac |
| 3901 | cagtagcagc ggctccaaag cctcggggcc gcccaaccct ccagcccagg gggacggcac |
| 3961 | cagcctctcc cccaactaca ccctggaatc cacgtcgggg aatgacggca agccggtctc |
| 4021 | cgggggcggc ggccggggac ggggtcgcag aaaaagggac agtggtcacg tgagccctgg |
| 4081 | caccttcttt gacaagtact cggcggctcc ggacagcggg ggcgcacctg gggtgagccc |
| 4141 | agggcagcag caagcgtcag gcgcagccgt cgggggaagc tccgcaggcg agacgcgcgg |
| 4201 | ggcaccgacg ccccacgaaa aggcgctcac gtcgccatcc tgggggaagg gggctgagtt |
| 4261 | gctcctgggg gatcagccgg acctcattgg gtccctggac ggcggggcca agtcggacag |
| 4321 | tagttcgcca aacgtgggtg agttcgcctc ggacgaggtg agcacgagct acgccaatga |
| 4381 | ggacgaggtg ttgtccagct ctgacaaccc ccaggcacta gttaaagcga gcaggagtcc |
| 4441 | cctggtgacc ggctcgccca aactccctcc ccgtggggta ggcgccgggg aacacggacc |
| 4501 | gaaggcgccc ccgcccgccc tcggcctggg catcatgtct aactctacct cgacccctga |
| 4561 | cagctacggc ggcggtgggg gcccgggcca tccgggcact ccgggcctgg agcaggtccg |
| 4621 | caccccgacg agcagcagcg gcgccccgcc acccgacgag atccaccccc tggagatcct |
| 4681 | tcaggcgcag atccagctac agaggcagca gttcagcatc tccgaggacc agcctctggg |
| 4741 | gctgaagggt ggcaagaagg gtgagtgcgc cgtcggggcc tcaggggcgc agaatggcga |
| 4801 | cagcgagctg ggcagctgct gctccgaggc ggtcaagagc gccatgagca ccattgacct |
| 4861 | ggactcgctg atggcagagc acagcgctgc ctggtacatg cccgctgaca aggccctggt |
| 4921 | ggacagcgcg gacgacgaca agacgttggc gccctgggag aaggccaaac cccagaaccc |
| 4981 | caacagcaaa gaagcccacg acctccctgc aaacaaggcc tcagcatccc agcctggcag |
| 5041 | ccacttgcag tgcctgtctg tccactgcac agacgacgtg ggtgacgcca aggctcgagc |
| 5101 | ctccgtgccc acctggcggt ccctgcattc tgacatctcc aacagatttg ggacattcgt |
| 5161 | ggctgcccta acttgaatga caagaaagat cccctcctct accaggccct tcctctcccc |
| 5221 | ctgtctgttt ccttccccct caaccttacc ccacccctct gttaatttga aagggccact |
| 5281 | attgctgagt ggatgagttt tttttttttc tctaggttgg tacctgctta gtggcatatg |
| 5341 | gaccggaaag ggttaattta aaggggggga acctcaaaag tttttttaaa aaagaaactt |
| 5401 | gtctgccaca gtatgttacc agtgttaacc cttctgcagt tagcaaactt ttgcttaagc |
| 5461 | ctttttcctc tagatactcc ccatgtttcg gtaatcttgg catacatttt ttagatgacc |
| 5521 | tctttccttg ttttgttttc atgctgctgt atgtccaagt attgttattt cataataaga |
| 5581 | caagagttgc tttctttttt attctttttc cttttcttac cccctcccct tttattttct |
| 5641 | ttttgctttg ttcactgctt attaaaatgg aaatcctgga gaatagtagt tctggaatat |
| 5701 | tgccgggtga aagtccaatt gtcatcacaa tgttatatat tgacacccca gtgtcatcag |
| 5761 | tcaggcagga gccaaacaat gaatgcccct cttaggtatt ccgcctggga ttttgttttg |
| 5821 | tctgttccct aagaaaatat attttcattc ctgcaaacac agtgctcagc cttcagttcc |
| 5881 | cttccacttg agttctctct tctcctgctg gaagccgccc ctctctgcga tcgacgtgag |
| 5941 | gacgtgtcca gctctgctct gtgggaagga gttggaatgt tcgacagcag tgttttctct |
| 6001 | ccttttctgg gcctcctcgc aaatgcccag gccctgcatt ttcacgctgt gctaagcagc |
| 6061 | ctttggtctg catgggggat ggtgtgctcc cagcctgcag tctttggagc aaggctgctg |
| 6121 | cccgtgcctt gggtgctgga gttggaggag gctgttctca gccctttccc ttttctgaaa |
| 6181 | gctgttcctg gccgggcatc ccagggaaga aggaggggac tgcgtgtatc tcctccacct |
| 6241 | ctcccattcc atccccagtc cagcctgggc aaccccaccc ctgggaggga tgaggcaccc |
| 6301 | tcttgctcag cctgctcagc cttctctgag cctttgcagg gatctgcaga ctcctgaggg |
| 6361 | ctagaggaca gagaaagaga atagaatgaa atgactttga ttcctgcgcc ttttagtttt |
| 6421 | gaactctgga attcctctgc cccctcccca acattttttt ggaatctcac cctgttgcaa |
| 6481 | aactagagcc atgtcccaag catctcacaa aggaataact gctctgagca gagatgagtg |
| 6541 | gtggttggca ggggcaggca actttgggtg ctgctgatgc ctgcaaaagc catttatggc |
| 6601 | ttgtggtggg gggcacatag attccccggt gggttagaca ggaagtaact gatatcactt |
| 6661 | cacccaaata tataaccgtg atggttatct atttaatttc agtttttgtt aacgagcgtg |
| 6721 | tcttactaaa acgctccact ttgagctccc ccaccccctc caggtcctca gagtttgcag |
| 6781 | atctgggctt tctaaagcaa gtgacctgaa ggctctgggc tcaccataca acacccacgt |
| 6841 | tgtttatttc aaagaacttt tcagcgaagg gagaggagct ttcagaaaaa cctcactctt |
| 6901 | tcccctccct tctcccctct ttccttctgc cggtcctttt ggctggggtc tgagtctgcg |
| 6961 | gttctcgcct gggcagtctt gacgaggagc aaaccccgcc ttcagagggc agacaaagca |
| 7021 | ggtggcatga attgatcagc gagaaaggtg tgagccgagg cagttcctgc gttctgctac |
| 7081 | aaaaggaatg gaaagggaag ggaatttccc cccaccatgg gctgtgggag agttgaccgt |
| 7141 | attctgggca agactccatg acccctctga ttctgcagtg tacagctgtt tgagagcctc |
| 7201 | atcattttac ttttgaaaca ggaatgattt ctccttaatt gcttaaggcc ggggagcaaa |
| 7261 | gtgtcttaac ttctgtcttt gactttccca gcgttgagtc atcaacactt tgccaattag |
| 7321 | ctcatggtcc tggcaacctc agaaacccct gaagttttaa aaactttctc gctccccacg |
| 7381 | accccagaat gaaacagctt taaaaatagc cttaagcaaa aggatgttat ttcattaaat |
| 7441 | ttggtttaat ggaaagaata aaagtaaatg aaaaacacac cctacacact agactccgaa |
| 7501 | cactggtaat cagtactgca tagcaaactc tttgggaaag aaaacgaaaa tgttattgca |
| 7561 | catgtaaaat atgaaaactt aactctgctg tgtgttaggc aatcctgtaa tcttttttga |
| 7621 | ctcttaaaag aaattcattt ctgaaatgct tggttggaag actgtgacaa tagctcatga |
| 7681 | aattgagtgt tatttttttc tttctttttt aaaaaaatat gtaaagtgca gtcttctgta |
| 7741 | ttcctgcata ttgtatatac ctgtatatgt tttcctgagc agttaaataa caataaatat |
| 7801 | gacgttaatg gtga |

An amino acid sequence for the protein (Mediator complex subunit 19 (Med19)) encoded by the MED19 gene that is a regulator of T cell exhaustion/dysfunction resistance is available from the UniPROT database at accession no. A0JLT2 or accession number NP_001304007 or NP_703151. cDNA NM_153450 or NM_001317078 with gene location at Chr 11: 57.7-57.71 Mb.

| (SEQ ID NO: 89) | |
|---|---|
| MENFTALFGAQADPPPPPTALGFGPGKPPPPPPPPAGGGPGTAP | |
| PPTAATAPPGADKSGAGCGPFYLMRELPGSTELTGSTNLITHYNLEQAYNKFCGKKVK | |
| EKLSNFLPDLPGMIDLPGSHDNSSLRSLIEKPPILSSSFNPITGTMLAGFRLHTGPLP | |
| EQCRLMHIQPPKKKNKHKHKQSRTQDPVPPETPSDSDHKKKKKKKEEDPERKRKKKEK | |
| KKKKNRHSPDHPGMGSSQASSSSSLR | |
| (SEQ ID NO: 90) | |
| 1 | ggcagacacg gagacagcgc cggggcggag ggtacgatgg agaatttcac ggcactgttt |
| 61 | ggggctcagg ctgacccacc accgccccca accgcactcg gcttcggacc aggaaagcct |
| 121 | ccacccccgc caccgcctcc tgcgggcggg ggacccggca cggccccgcc tcccaccgcg |
| 181 | gccacggctc ctcctggcgc ggacaagtca ggagctggct gtggcccttt ttacctcatg |
| 241 | agggaactgc caggtagcac agagctgaca ggcagcacga atctgatcac acactacaac |
| 301 | ttggaacaag cctataataa attctgtggg aagaaggtga aggagaagct aagtaacttc |
| 361 | ctgcctgacc tgccagggat gattgatctg cctggttccc atgataacag cagcctccgc |
| 421 | tctctcattg agaagccccc tattctcagt agctctttca atcctatcac agggaccatg |
| 481 | ctggccggct tccgcctcca cactggcccg ttgccggagc agtgtcgtct gatgcatatt |
| 541 | cagcctccca agaagaagaa taagcacaag cacaaacaga gccgtaccca ggatcctgtc |
| 601 | cccccagaaa caccatctga ttcagatcac aagaagaaga aaaagaaaaa agaagaggat |
| 661 | cctgaacgga aaaggaagaa gaaagagaag aagaaaaaga agaatcgaca tagtccagac |
| 721 | cacccaggta tgggcagctc ccaggccagc agcagcagca gcctacgcta ataggaccac |
| 781 | tggactcttt gccaggatgg cttttcctgc tgtactgaac ctgctgataa aagctgcctt |
| 841 | ccaggctctt ggacactgcc ttgggagcat cctgcagctg ggacagaggc cagctcctgt |
| 901 | tgggctcagt tgagactaag taaattagga gagagaggga catgcttttg taggctcatc |
| 961 | ccagttggtt ttctcatgga catctcttcc tctcccagga agcttacaat tttcttctct |
| 1021 | ctcttttgtg caatttgtct gatttaggac ttgttctgtg ttttctttaa aaaaaaaaaa |
| 1081 | attacattta tcaaactgg |

An amino acid sequence for the protein (Homeobox protein MOX-1) encoded by the MEOX1 gene that is a regulator of T cell exhaustion/dysfunction resistance is available from the UniPROT database at accession no. P50221 or accession number NP_001035091, NP_004518 or NP_054705, cDNA NM_001040002, NM_004527 or NM_013999 with gene location at Chr 17: 43.64-43.66 Mb.

| (SEQ ID NO: 91) | |
|---|---|
| MDPAASSCMRSLQPPAPVWGCLRNPHSEGNGASGLPHYPPTPFS | |
| FHQKPDFLATATAAYPDFSASCLAATPHSLPQEEHIFTEQHPAFPQSPNWHFPVSDAR | |
| RRPNSGPAGGSKEMGTSSLGLVDTTGGPGDDYGVLGSTANETEKKSSRRRKESSDNQE | |
| NRGKPEGSSKARKERTAFTKEQLRELEAEFAHHNYLTRLRRYEIAVNLDLSERQVKVW | |
| FQNRRMKWKRVKGGQPISPNGQDPEDGDSTASPSSE | |
| (SEQ ID NO: 92) | |
| 1 | agccctctgc aggcatttgc tctgggctcc aggacgaact cctcgtcagc tggtgcgtct |
| 61 | gggttgggac agtgaaaatg tttagtgtca ttggcgacaa atacacatac aggggatccc |
| 121 | agtaggtagg ctggtgcatt ggaggggcag gagcaagccc gacataggtg tgtgcacaca |
| 181 | cataggagta gctggccaca tgtgtgtgta ctttaaggag agactttagt tttgggtttt |
| 241 | tttttttttt tggttcctgg ggtaaatttt ctgttgaaca tttttccctc ctatttagtt |
| 301 | ttttttcttt tgcattttta aaaatttgaa cataaaaagt ataaagaatc aaatctttga |
| 361 | aaggaccgag gcgtgcagcg gacagcagat ggatcccgcg gccagcagct gcatgaggag |
| 421 | cctccagccc ccagcccctg tctggggctg ccttcgaaac ccccactcgg aaggcaatgg |
| 481 | ggcctcaggg ctaccccact acccgcccac cccgttctcc ttccaccaga aaccagactt |
| 541 | cctggcgaca gcgacggcag cgtaccctga cttctcagcc tcctgcctgg cagccacccc |
| 601 | acacagcctg ccccaggagg agcacatctt cactgagcag caccccgctt tcccacagtc |
| 661 | ccccaactgg cacttccctg tctcagacgc ccggcgcagg cccaactcag gcccggcagg |
| 721 | gggttccaag gaaatgggga ccagcagcct gggcctggtg gacaccacag gaggcccagg |
| 781 | cgatgactac ggggtgcttg ggagcactgc caatgagaca gagaagaaat catccaggcg |
| 841 | gagaaaggag agttcagaca accaggagaa cagagggaag ccggagggca gcagcaaagc |
| 901 | ccgraaggag aggacggcct tcaccaagga gcagctgcga gagctggagg cagagtttgc |
| 961 | ccatcataac tacctgactc ggctccgcag atatgagatt gcggtaaacc tggacctctc |
| 1021 | tgagcgccag gtcaaagtgt ggttccagaa ccgaaggatg aagtggaagc gtgtgaaggg |
| 1081 | aggtcagccc atctccccca atgggcagga ccctgaggat ggggactcca cagcctctcc |
| 1141 | aagttcagag tgagattctg catggaggaa aaatgactaa ggactgagcc ccctacccaa |
| 1201 | ctacccccac cccaatccca ccttcaccct cttccttccc cagccagggc agcctctcca |
| 1261 | catctttccc tgactcttgg atatgaaact gcccagcatt cctgggagtc ttaggatttt |
| 1321 | ctaggaagtt ctgtccagcc tcttagcagc ctcttcccta gggcctttgc tcccacactc |
| 1381 | tcatggaatc agacagagat cctaccgggc cggatgaatc tggaaacagc ttcagagata |
| 1441 | ctgcttctca gcgtctcttg gctgccaccc atgcctcctc ctaccgctgt tctcctaggt |
| 1501 | cagccaggcc tcctcctggt ctggacacca cctggcctgg tgggagagga gctttggaac |
| 1561 | cagctggcga ctcggaaagt aaatgcttca aaaggaagga aatgacagag acacacgccc |
| 1621 | ttgcccacct tcctctgtag gctgcacatc tgaggctttg gggcccctta gttgtcccga |
| 1681 | aaccccaaga aaaatcagaa tgaggagagt caaggacagc aactcagctg ctgcaagcca |
| 1741 | gaaacacatc cctgtctcca aatttgttgg ctaagtggag acacttctga gaactgacta |
| 1801 | gagaagacag aaaaatagcc cgatgtaggt ttcggtgtcc ccatataggc ccgtccacac |
| 1861 | aggcttgact gggtggacaa gaatgaaccc atgacagcac ctgctgcttc aaaatcaaaa |
| 1921 | tcaatttagg gatacagcag gggctgttgg gctgtgctcc agagaaaagg agcagctact |
| 1981 | ccttttaaat ccacgatttc tggattgaaa acctgtccag atgctgagtt gttgggctga |
| 2041 | acaactagga gctgaaaaca acgtagaggc tggaaagtgt cccctgcatt ctggagggga |
| 2101 | ggggagataa taaggagggc tgctgggtga gggcctggag atgtggaacc ctggagtgga |
| 2161 | aggttctcca gtgacagtgt cctgtgactg caaaagggga caagaaaatc cctcttcctc |
| 2221 | catgggatgg atttaagctc ttgctgtgtg ttctacaaat gctgttattg tgggaggaaa |
| 2281 | tgctaggttt ttgtgtgtgg actgcccaga cctcagccag gtcttctgga gatgacattt |
| 2341 | gaggactgat ggccaaagag catgggggac tgaagccctg gctgcctcag cgctctgtct |
| 2401 | cccaacacca gctggtgttg cagagggagg tcaacgtgag tttggatctc ttgtacgcag |
| 2461 | atgtaatcat tcacatgtaa aaataacccc acctccccac cccaaaaagg gcaagagctg |
| 2521 | tggaaaatga ttgccaaatg agatggctgg ttagagcatg attttttcta aagcatactt |
| 2581 | catatatttt cttaagatta catcaagcta attgtgcgag ctcaattcac tttgtaagaa |
| 2641 | aactctcgga gaaataaaat caataaaaag caaa |

An amino acid sequence for the protein (Adenylyltransferase and sulfurtransferase MOCS3) encoded by the MOCS3 gene that is a regulator of T cell exhaustion/dysfunction resistance is available from the UniPROT database at accession no. 095396 or accession number NP_055299, cDNA NM_014484 with gene location at Chr 20: 50.96-50.96 Mb.

| (SEQ ID NO: 93) | |
|---|---|
| MASREEVLALQAEVAQREEELNSLKQKLASALLAEQEPQPERLV | |
| PVSPLPPKAALSRDEILRYSRQLVLPELGVHGQLRLGTACVLIVGCGGLGCPLAQYLA | |
| AAGVGRLGLVDYDVVEMSNLARQVLHGEALAGQAKAFSAAASLRRLNSAVECVPYTQA | |
| LTPATALDLVRRYDVVADCSDNVPTRYLVNDACVLAGRPLVSASALRFEGQITVYHYD | |
| GGPCYRCIFPQPPPAETVTNCADGGVLGVVTGVLGCLQALEVLKIAAGLGPSYSGSLL | |
| LFDALRGHFRSIRLRSRRLDCAACGERPTVTDLLDYEAFCGSSATDKCRSLQLLSPEE | |
| RVSVTDYKRLLDSGAFHLLLDVRPQVEVDICRLPHALHIPLKHLERRDAESLKLLKEA | |
| IWEEKQGTQEGAAVPIYVICKLGNDSQKAVKILQSLSAAQELDPLTVRDVVGGLMAWA | |
| AKIDGTFPQY | |
| (SEQ ID NO: 94) | |
| 1 | acgacaactt ccggaagagg tcgccatggc ttcccgggag gaggtactcg ccttacaagc |
| 61 | tgaagttgcc caacgtgagg aggaattgaa ttcgctgaag cagaagctgg cgtcggctct |
| 121 | tttggctgag caggaaccgc agccagaacg gctggttccg gtgtcgccgc tgccgccgaa |
| 181 | ggccgctctg tcccgagatg agattctgcg ctatagccgg cagctagtgc tgcccgagct |
| 241 | gggcgtgcac ggacagctgc gcctggggac cgcgtgcgtg ctaatcgtgg gctgcggtgg |
| 301 | gctcggctgt ccactagcgc agtacttggc agcggccggc gtgggccgcc ttggccttgt |
| 361 | ggactatgac gtggtagaga tgagcaacct ggcccgccaa gtgctgcatg gcgaggcact |
| 421 | ggctggccag gccaaggcct tttcggccgc cgcctcgctg cgccgcctca attcggcagt |
| 481 | ggaatgcgtg ccgtacactc aggcccttac gccagccact gccctagacc tggtccgccg |
| 541 | atatgatgtg gtggctgact gctcggacaa cgtgcccact cgctacctgg ttaatgacgc |
| 601 | atgtgtgctg gcgggtcggc ccctcgtgtc tgccagtgcc ttgcgcttcg agggccaaat |
| 661 | cacagtctac cattatgacg gtggcccttg ctatcgctgc atattccccc aaccaccccc |
| 721 | agcggagaca gtgaccaact gcgcggacgg cggggtgctc ggtgtcgtta ccggggtcct |
| 781 | gggctgcctg caggccttgg aagtgctgaa aatcgctgcg ggtctgggcc cctcttacag |
| 841 | tggcagcttg ttgctctttg atgccctgag agggcatttc cgctctattc ggctgcggag |
| 901 | ccgcaggctc gactgtgcag cttgcgggga acggcccact gtgactgatc tgctggacta |
| 961 | tgaagccttc tgtggctcct cagccactga taaatgccgc tccctgcaac tactgagccc |
| 1021 | agaggagcgt gtttctgtca ccgactataa gcgactgctg gattctgggg cattccacct |
| 1081 | gttgctggac gtcaggcctc aggtggaggt ggacatttgt cgtttgcctc atgccctaca |
| 1141 | catccctctg aaacatttgg aacgcaggga tgcggagagc ctgaaactct taaaagaagc |
| 1201 | aatctgggaa gagaagcagg gcacacaaga aggggctgct gtccccattt atgtgatttg |
| 1261 | caaactggga aatgactcac agaaagccgt gaagatcctc cagtccttat cagcagctca |
| 1321 | agagttagac cctttaacag ttcgggatgt tgtggggggc ctcatggcct gggctgccaa |
| 1381 | aatcgatgga acatttccac agtactgagg tgactggtat agtctgatga gaaagatgtg |
| 1441 | gattgccata atacctcaaa gatacacttg tttgcatttt tcggtaatat acataggagc |
| 1501 | tggggattct acagtatctg tgaatacgtg gactcctttt tataaggagt tttaaaaatt |
| 1561 | gttatgtatt ggatgaatga cttattaatg gattataccg tttctgagaa ccatcatttt |
| 1621 | ttttttcagc acacggagga tgtcttggac atgtgagatg taacgtgaca ggattttgca |
| 1681 | ttttaaactg cagatcattt acatgtcctt attttccacc tcccccagtc aaaatgcttt |
| 1741 | ctaaatcatt ttcacagatt atatacttcg gatctgttta ctgttcagtt aagaaattct |
| 1801 | tggatcttat taatatttca gatgatagaa tacatcctac agaaatacat gtttaaaatg |
| 1861 | taaattgttt taattattca gaaagagggt cattctattt ggccttttga attagtatca |
| 1921 | aaatgagatt tttttttttt ttttgagatg gaatctctct ctgttgccca ggctggagtg |
| 1981 | tagtggtgcg atctcagctc actgcaacct ccgcctccca ggttcaagcg attctcctgc |
| 2041 | ctcagcctcc tgagtagctg ggattacagg cgcacgccac cacgcctggc taatttttgt |
| 2101 | atttttagta cagagggggt ttcaccatgt ttgtcaggct ggtctcgaac tcctgacctc |
| 2161 | gtgatccgcc tgcctcgact tcccagagtg ctgggattac aggcgtgagc caccacaccc |
| 2221 | tcccgagatc tttaaagttt accaaaataa aggaaaccac ctttgtagtc tgtcagtgtc |
| 2281 | tcttataaat ttctactgtt ttattaaagt taacttcagg gtccacctta tgttgcacac |
| 2341 | atatcacatc gtttaaattg catactatgg tttgacttgc tttggaattt tcaagtcaaa |
| 2401 | gtcccaaccc tttgcctctc cccctttaat ttgccttttc tcttttgatt taatgttttt |
| 2461 | ttattccagc attttatact aatgtaaaat gtttttatgt ttgtttactt aacattttca |
| 2521 | taaaatagta ggtagtctca ccaacttcca atatatacaa caacattata agtaaaaatt |
| 2581 | tgatttgtga gtgcctatca gtggccagat gaactaatgg cttgtcagaa gggggcctgt |
| 2641 | gacctcctgc ttgctgcatg caggcttata atcaccaatt gctgctgttg tttttttaag |
| 2701 | aatggcagaa atcacagata caactataaa atctttgtat ttctgttaac catttttttt |
| 2761 | aaagcttaaa tgtatcataa gccagcaaaa accccttgta gaccagattt acttaccagt |
| 2821 | ttgcaatttg tgatatagaa tcttgtttta ttataagagg gtggtattta acaaattcaa |
| 2881 | aattagtcat cactttttaa ctgtgaaaca ttgtaactaa ttctttgaac aggtatactt |
| 2941 | aaagtatggt atacccttct gttataaaga tgaagcaggt ctcttatgac tgttcctgcc |
| 3001 | aacgagttat ggaggtaaat gactgtgacc tggagtggag aacaaagatg gctggcctgt |
| 3061 | gtttttcatc tccttcttcc agggacaact gattctgata catagcagga aacaaatagc |
| 3121 | gacacacagg aacaatttca tattctaaag actgaagtcc taaagttata gacagttgta |
| 3181 | gttcctataa acctggttgt cacaggggac aggcaagaga agctgactag ctaagtgtta |
| 3241 | aaaggtttac taaagtgata ggaggtgctt ttgagaattt tggggacaga ttcaaggctg |
| 3301 | tacttctagg aatgatgccc gaaatcatgc acaaaactgg ccttaggaga acagtgtcta |
| 3361 | tgttgtgctg ccctatcaag taccggagga aacgccacta cagtctcacc agtttcactt |
| 3421 | gacgtgcccc cccaactctc ccagaaagcc caacacttca gccacagtcc atgtgtcgag |
| 3481 | aaacagagtc actacattat tcttttctga gtcaagcctt atgagggtgc ctggtaggca |
| 3541 | gagtctaatt acatgcctac atcttagctg caagagaggc tgggaatttg tgttttgata |
| 3601 | tctacaatag gaagacagaa ctcatgatgt gggattttcc caaatagtga aatatttttc |
| 3661 | aaaagatggg ctgttaagaa tataacagat acccagtaca caacccagat ctatttcagt |
| 3721 | atttgttttt tcttgttgtt gttttggaga cagagtctcg ctctatcccc caggctggat |
| 3781 | ggagtgcagc ggcacaatct cggctcactg caacctccac ctcccatgtt caagtgattc |
| 3841 | tcatgcctca gcctcctgag tagctgggat tacaggcacc tgccaccaca tctggctaat |
| 3901 | ttttctattt ttagtagcga tggggtttcg ccatgttggc ctggctggtc ttgaactcct |
| 3961 | gacctcaggt gatctgtgca cctcagcctc ccaaagtgct aggattacag gcatgagtca |
| 4021 | ctgcacctgg catatattag tatttgaaat ttggttttct ctggggattg tagctaggtt |
| 4081 | tttatggtga gataggattc gaacataatt tgttttttga gacgaagtct tgcttcgtcg |
| 4141 | cccagggtgg agtgtagtgt tgtgatcaca gctcgctgtg gcctcaacct cctgggttca |
| 4201 | agtgatcctc ccacctcagc ctctcaagta gcttgaatta cagttgtatg ccaccatgcc |
| 4261 | tagccatttt ttaatttaaa aattttttgg tagcaatggg gtctgaatat gtggcctagg |
| 4321 | ctgatctcaa actcctgggc tcaagcatcc ctcctgcctc agcctcccaa agtgcttggg |
| 4381 | attacgggta tgaggcacca tgcccagcct gaacttgatt tttttgttgt ttccttttca |
| 4441 | taacaaacat caaatgcctt cagtgtacta gttcctctag gttctggaat aaaaatgagc |
| 4501 | aagaggtttc ctgttgtaga ggagtttata tttaaatggt gataagtaaa ttactgataa |
| 4561 | gtgccataaa agaaatgaaa ctgtgacata ttagggtggg ggaggggatc tgttttgaat |
| 4621 | agcttaatta tggaaggcct cagagatgac attaaaccca aaacctgaaa ggtgagaaag |
| 4681 | agccagttac agaagatctg aggaaaattc tatactggga gaaaaataac tgtaaagacc |
| 4741 | tcaaggtaaa taaacttgga atgttccgtt caagaaactg cagaggcaag ttgggttgca |
| 4801 | aacaattagg gagagtggga cagggtgagg tttgagaagc agggaacatc ataaactttg |
| 4861 | tagaccatag tagtttggtt tttattctaa atacaataat agctaatatt ttgagcactt |
| 4921 | actgtgttat ttgggatttg aatccagatg ctctggttga agagcccatt ccctaagtgt |
| 4981 | catgctatac tgactctaag caaaactttg ccattgcttc atatgtgttc atgccctgtc |
| 5041 | agggcataga aagcaatcta tgtcctgaca atcacggaag tgtattccag atacaataga |
| 5101 | aatgtactct ga |

An amino acid sequence for the protein (NAD(P)H dehydrogenase, quinone 2, also known as QR2) encoded by the NQO2 gene that is a regulator of T cell exhaustion/dysfunction resistance is available from the UniPROT database at accession no. P16083 or accession number NP_000895, NP_001277150, NP_001277151 or NP_001305869, cDNA NM_000904, NM_001290221, NM_001290222 or NM_001318940 with gene location at Chr 6: 2.99-3.02 Mb.

| (SEQ ID NO: 95) | |
|---|---|
| MAGKKVLIVYAHQEPKSFNGSLKNVAVDELSRQGCTVTVSDLYA | |
| MNLEPRATDKDITGTLSNPEVFNYGVETHEAYKQRSLASDITDEQKKVREADLVIFQF | |
| PLYWFSVPAILKGWMDRVLCQGFAFDIPGFYDSGLLQGKLALLSVTTGGTAEMYTKTG | |
| VNGDSRYFLWPLQHGTLHFCGFKVLAPQISFAPEIASEEERKGMVAAWSQRLQTIWKE | |
| EPIPCTAHWHFGQ | |
| (SEQ ID NO: 96) | |
| 1 | gcaggttccg ggctgcttag gttggcaccg gtccgtggtc cccgggggcg cagtcgcagc |
| 61 | gctcccgccc tccaggcgtc agcgagtgcg cggtccagtg cggccggaac ctggcgcaac |
| 121 | tcctagagcg gtccttgggg agacgcgggt cccagtcctg cggctcctac tggggagtgc |
| 181 | gctggtcgga agggggatgg agacagaagt cacaaactca catgccacca gccatatgat |
| 241 | gtaaacatgt aaactgggcc aaggaataca aaagggagtg gtgagctggg gacactgaag |
| 301 | aatggatgcc ctgtctagag gagtgaaaat gaagcatttc atacaaacaa catgatttcg |
| 361 | ggcaatcact ttgggaatca ggataaggtg ttcccccagt ctgcagaaac actggcaaca |
| 421 | tttacaaagc tttaggttca ttcttgtaag tgttgctggt gtcagaatag gaacctccct |
| 481 | gcgctttgaa gggatgaatg tgacctctcc cacattctcc ggtgctgtga tgtaccttaa |
| 541 | acacagcaag ggcacctggc ctgggtggag cccactcctc agcacccacc tcacttcttg |
| 601 | cagtattctg cagaccccag ccctgtgcct gtgctcctgg acagctggag ataaggagtg |
| 661 | ggccctggaa gatgctcatt caggccctgc traagattcc agtcctgatt gctggactcg |
| 721 | ctgaagagag actacgcagg aaagccccag ccacccatca aatcagagag aaggaatcca |
| 781 | ccttcttacg ctatggcagg taagaaagta ctcattgtct atgcacacca ggaacccaag |
| 841 | tctttcaacg gatccttgaa gaatgtggct gtagatgaac tgagcaggca gggctgcacc |
| 901 | gtcacagtgt ctgatttgta tgccatgaac cttgagccga gggccacaga caaagatatc |
| 961 | actggtactc tttctaatcc tgaggttttc aattatggag tggaaaccca cgaagcctac |
| 1021 | aagcaaaggt ctctggctag cgacatcact gatgagcaga aaaaggttcg ggaggctgac |
| 1081 | ctagtgatat ttcagttccc gctgtactgg ttcagcgtgc cagccatcct gaagggctgg |
| 1141 | atggataggg tgctgtgcca gggctttgcc tttgacatcc caggattcta cgattccggt |
| 1201 | ttgctccagg gtaaactagc gctcctttcc gtaaccacgg gaggcacggc cgagatgtac |
| 1261 | acgaagacag gagtcaatgg agattctcga tacttcctgt ggccactcca gcatggcaca |
| 1321 | ttacacttct gtggatttaa agtccttgcc cctcagatca gctttgctcc tgaaattgca |
| 1381 | tccgaagaag aaagaaaggg gatggtggct gcgtggtccc agaggctgca gaccatctgg |
| 1441 | aaggaagagc ccatcccctg cacagcccac tggcacttcg ggcaataact ctgtggcacg |
| 1501 | tgggcatcac gtaagcagca cactaggagg cccaggcgca ggcaaagaga agatggtgct |
| 1561 | gtcatgaaat aaaattacaa catagcta |

An amino acid sequence for the protein (PR domain zinc finger protein 14) encoded by the PRDM14 gene that is a regulator of T cell exhaustion/dysfunction resistance is available from the UniPROT database at accession no. Q9GZV8. cDNA NM_024504.4 with gene location at 55 Chromosome 8: 70,051.651-70.071,693.

| (SEQ ID NO: 97) | |
|---|---|
| MALPRPSEAVPQDKVCYPPESSPQNLAAYYTPFPSYGHYRNSLATVEEDFQPFRQLEAAASAAPAMPPFPFRMAPPL | |
| LSPGLGLQREPLYDLPWYSKLPPWYPIPHVPREVPPFLSSSHEYAGASSEDLGHQIIGGDNESGPCCGPDTLIPPPP | |
| ADASLLPEGLRTSQLLPCSPSKQSEDGPKPSNQEGKSPARFQFTEEDLHFVLYGVTPSLEHPASLHHAISGLLVPPD | |
| SSGSDSLPQTLDKDSLQLPEGLCLMQTVFGEVPHFGVFCSSFIAKGVRFGPFQGKVVNASEVKTYGDNSVMWEIFED | |
| GHLSHFIDGKGGTGNWMSYVNCARFPKEQNLVAVQCQGHIFYESCKEIHQNQELLVWYGDCYEKFLDIPVSLQVTEP | |
| GKQPSGPSEESAEGYRCERCGKVFTYKYYRDKHLKYTPCVDKGDRKFPCSLCKRSFEKRDRLRIHILHVHEKHRPHK | |
| CSTCGKCFSQSSSLNKHMRVHSGDRPYQCVYCTKRFTASSILRTHIRQHSGEKPFKCKYCGKSFASHAAHDSHVRRS | |
| HKEDDGCSCSICGKIFSDQETFYSHMKFHEDY | |
| (SEQ ID NO: 98) | |
| 1 | gcccgaagtc taccgagccc gagcggccta ccgagcccga gtggccccgc agcgtccagg |
| 61 | aggcgcccgc tccgcggtgg cgctcttgga ggtggtgtcg gagagccgcc gagcgtgcgg |
| 121 | tcccgggatg gctctacccc ggccaagtga ggccgtgcct caggacaagg tgtgctaccc |
| 181 | gccggagagc agcccgcaga acctggccgc gtactacacg cctttcccgt cctatggaca |
| 241 | ctacagaaac agcctggcca ccgtggagga agacttccaa cctttccggc agctggaggc |
| 301 | cgcagcgtct gctgcccccg ccatgccccc cttccccttc cggatggcgc ctcccttgct |
| 361 | gagcccgggt ctgggcctac agagggagcc tctctacgat ctgccctggt acagcaagct |
| 421 | gccaccgtgg tacccaattc cccacgtccc cagggaagtg ccgcccttcc tgagcagcag |
| 481 | ccacgagtac gcgggtgcca gcagtgaaga tctgggccac caaatcattg gtggcgacaa |
| 541 | cgagagtggc ccgtgttgtg gacctgacac tttaattcca ccgccccctg cggatgcttc |
| 601 | tctgttacct gaggggctga ggacctccca gttattacct tgctcaccca gcaagcagtc |
| 661 | agaggatggt cccaaaccct ccaaccaaga agggaagtcc cctgctcggt tccagttcac |
| 721 | ggaggaggac ctgcacttcg ttctgtacgg ggtcactccc agcctggagc acccagccag |
| 781 | cctgcaccat gcgatttcag gcctcctggt ccccccagac agctctggat ctgattctct |
| 841 | tcctcaaact ctggataaag actcccttca acttccagaa ggtctatgcc tcatgcagac |
| 901 | ggtgtttggt gaagtcccac attttggtgt gttctgcagt agttttatcg ccaaaggagt |
| 961 | caggtttggg ccctttcaag gtaaagtggt caatgccagt gaagtgaaga cctacggaga |
| 1021 | caattctgtg atgtgggaga tctttgaaga tggtcatttg agccacttta tagatggaaa |
| 1081 | aggaggtacg gggaactgga tgtcctatgt caactgtgcc cgcttcccca aggagcagaa |
| 1141 | cctagttgct gtgcagtgtc aagggcatat attttatgag agctgcaaag agatccatca |
| 1201 | gaaccaagag ctccttgtgt ggtatggaga ctgctatgag aaatttctgg atattcctgt |
| 1261 | gagccttcag gtcacagagc cggggaagca gccatctggg ccctctgaag agtctgcaga |
| 1321 | aggctacaga tgtgaaagat gtgggaaggt atttacctac aaatattaca gagataagca |
| 1381 | cctcaagtac accccctgtg tggacaaggg cgataggaaa tttccctgtt ctctctgcaa |
| 1441 | acgatccttt gagaagcggg accggcttcg gatccacatt cttcatgttc atgagaagca |
| 1501 | ccggcctcac aagtgttcta catgtgggaa atgtttctct caatcttcca gcctaaacaa |
| 1561 | acacatgcga gtccactctg gagacagacc ataccagtgt gtgtattgta ctaagaggtt |
| 1621 | cacagcctcc agcatactcc gcacacacat caggcagcac tccggggaga agcccttcaa |
| 1681 | atgcaagtac tgtggtaaat cttttgcatc ccatgctgcc catgacagcc atgtccggcg |
| 1741 | ttcacacaag gaggatgatg gctgctcatg cagcatctgt gggaaaatct tctcagatca |
| 1801 | agaaacattc tactcccaca tgaagtttca tgaagactac tagccctgcc aggcacaatg |
| 1861 | actcacgcct gtaatcccag cactttggga ggcagaggtg ggtggatcac tcaagtccag |
| 1921 | gagttcgaga ccagcctggg caacatggtg aaatcctgtc tctaccaaaa aaatacaaaa |
| 1981 | atcagctggg ggtggtggca catgcctgtg gttccagcca ctcaggaggt cgaggtggca |
| 2041 | ggatggtttg agcacaggag acggaggttg ctgtgagctg agatcgcccc actgcttttc |
| 2101 | aacctgggtg acagaaccag accctgtctc aaaacaaaac aaaacaaaaa aaatgagtag |
| 2161 | ccctcaagag tgtggagaca atgtaaaaac aagagattcg gattctctct atttcctttt |
| 2221 | atgggttata gaagtccctg cagttggctg tgtgtggtgg ctcacgcct |

An amino acid sequence for the protein (Synaptonemal complex central element protein 1-like) encoded by the SYCE1L gene that is a regulator of T cell exhaustion/dysfunction resistance is available from the UniPROT database at accession no. A8MT33, cDNA NM 001129979.3 with gene location at Chromosome 16.

| (SEQ ID NO: 99) | |
|---|---|
| MAGKLKPLNVEAPEATEEAEGQAKSLKTEDLLAMVIKLQKEGSLEPQIEDLISRINDLQQAKKKSSEELRETHSLWE | |
| ALHRELDSLNGEKVHLEEVLGKKQEALRILQMHCQEKESEAQRLDVRGQLEDLMGQHKDLWEFHMLEQRLAREIRAL | |
| ERSKEQLLSERRLVRAKLREVERRLHSPPEVEGAMAVNDGLKAELEIFGEQVRSAPEVGAGEGEAGPELPRARDEED | |
| PEPPVAAPDAL | |
| (SEQ ID NO: 100) | |
| 1 | accagtcatc aagcgaggct cgcgcgcagg ccccgcgttg gaaaatggcg gggaagctga |
| 61 | aacctctgaa tgtggaggcg ccagaagcta ctgaggaggc tgaagggcaa gccaagtctt |
| 121 | tgaagactga agacttgctg gcaatggtga taaagctgca gaaagaggga agcctggagc |
| 181 | cacagataga ggacctgatt agccggatta atgatcttca gcaagcaaag aagaaatcca |
| 241 | gtgaggaact gagagagacc cacagtctct gggaggccct gcatagggaa ttagactcct |
| 301 | tgaatggaga gaaagtgcac ctagaggagg tcttgggcaa aaagcaagag gcactgagga |
| 361 | tcctccagat gcactgccaa gagaaggaaa gcgaggctca gaggttggat gtcagaggac |
| 421 | agctggagga tctgatgggc cagcacaagg acctctggga attccacatg ctggagcagc |
| 481 | gactggcccg ggagatccgt gccctggaga gaagcaagga gcagctgctc tcggagagga |
| 541 | ggctggtgcg cgccaagctg cgggaggtgg agcggcggct gcactcgccg cctgaggtcg |
| 601 | agggcgccat ggcggtgaat gacgggctga aggcggagct ggagatattc ggggagcagg |
| 661 | tccggagcgc ccccgaggtc ggggccggcg agggagaggc cggacctgag ctcccccgcg |
| 721 | ctcgcgacga ggaggatccc gagccgccgg tggctgcccc tgacgccctc taggccagca |
| 781 | ggacccgccc gttcccgacc ttccctcgag acccgccaag aaataaaggc gatgatttcc |
| 841 | gaccatgctc gcgttctccg cggagtctgt gctacaccgt ggagcggggc ggggcgtgct |
| 901 | gggatctcga ggcggggcct ctgccggacc cctcccacca gtcgagcccc gggcgccgta |
| 961 | cagaggctgg gtaaatgctc ctgaactcag agagagtaag tgggtgtcag tatcccccgc |
| 1021 | cccacggcct catctcgagc tcccaaacca tctatgt |

An amino acid sequence for the protein (Synaptotagmin-like 2) encoded by the SYTL2 gene that is a regulator of T cell exhaustion/dysfunction resistance is available from the UniPROT database at accession no. Q9HCH5 or accession number NP_001156423, NP_001156424, NP 001156425, NP_001276537 or NP_001276538, cDNA NP 001156423, NP_001156424, NP 001156425. NP 001276537 or NP 001276538 with gene location at Chr 11: 85.69-85.81 Mb.

| (SEQ ID NO: 101) | |
|---|---|
| MIDLSFLTEEEQEAIMKVLQRDAALKRAEEERVRHLPEKIKDDQ | |
| QLKNMSGQWFYEAKAKRHRDKIHGADIIRASMRKKRPQIAAEQSKDRENGAKESWVNN | |
| VNKDAFLPPELAGVVEEPEEDAAPASPSSSVVNPASSVIDMSQENTRKPNVSPEKRKN | |
| PFNSSKLPEGHSSQQTKNEQSKNGRTGLFQTSKEDELSESKEKSTVADTSIQKLEKSK | |
| QTLPGLSNGSQIKAPIPKARKMIYKSTDLNKDDNQSFPRQRTDSLKARGAPRGILKRN | |
| SSSSSTDSETLRYNHNFEPKSKIVSPGLTIHERISEKEHSLEDNSSPNSLEPLKHVRF | |
| SAVKDELPQSPGLIHGREVGEFSVLESDRLKNGMEDAGDTEEFQSDPKPSQYRKPSLF | |
| HQSTSSPYVSKSETHQPMTSGSFPINGLHSHSEVLTARPQSMENSPTINEPKDKSSEL | |
| TRLESVLPRSPADELSHCVEPEPSQVPGGSSRDRQQGSEEEPSPVLKTLERSAARKMP | |
| SKSLEDISSDSSNQAKVDNQPEELVRSAEDVSTVPTQPDNPFSHPDKLKRMSKSVPAF | |
| LQDESDDRETDTASESSYQLSRHKKSPSSLTNLSSSSGMTSLSSVSGSVMSVYSGDFG | |
| NLEVKGNIQFAIEYVESLKELHVFVAQCKDLAAADVKKQRSDPYVKAYLLPDKGKMGK | |
| KKTLVVKKTLNPVYNEILRYKIEKQILKTQKLNLSIWHRDTFKRNSFLGEVELDLETW | |
| DWDNKQNKQLRWYPLKRKTAPVALEAENRGEMKLALQYVPEPVPGKKLPTTGEVHIWV | |
| KECLDLPLLRGSHLNSFVKCTILPDTSRKSRQKTRAVGKTTNPIFNHTMVYDGFRPED | |
| LMEACVELTVWDHYKLTNQFLGGLRIGFGTGKSYGTEVDWMDSTSEEVALWEKMVNSP | |
| NTWIEATLPLRMLLIAKISK | |
| (SEQ ID NO: 102) | |
| 1 | agtgccgggc gacacccggg ccgtgccgcc agcgtccgtc gggagagtga aggctcgcaa |
| 61 | cgccttgccc gggggaaggt aacgcgccgc gcccggggct gcgcggtttg caccgccagg |
| 121 | ccgagggctg ggggccagaa gacaagagag acggcgaggc gcgctccctg cccggcagcg |
| 181 | actcggaggt aggtgatgct ctgatgctgc tgcctcttat gaattgacga caggtgcaga |
| 241 | gagagaataa actgtgtcta agactggacc agagagtgag tgtgctggaa gccaccatag |
| 301 | gaacaagcga cctgtttggc aaggaataca gcatccttct gagagcctct tctcgacact |
| 361 | atcccacaga ccaggacttc ctcatgggaa agagcactta ggatcttgct atcagctgcc |
| 421 | ctaaactttt gctgagccaa aataagactt thatgcagaa ctgaacaaga tatttcattt |
| 481 | ttgtgtgttt taatcttcct ggttgcagtc ttagttgttc agccctgaga attttgagcc |
| 541 | acatttgttg ctattatttt tgcatgcact tttcaaaatg attgacttaa gcttcctgac |
| 601 | tgaagaggaa caagaggcca tcatgaaggt tttgcagcgg gatgctgctc tgaagagggc |
| 661 | cgaagaagag agagtcagac atttgcctga aaaaattaag gatgaccagc agctgaagaa |
| 721 | tatgagtggc caatggtttt atgaagccaa ggcaaaaagg cacagggaca aaatccatgg |
| 781 | cgcagatatc atcagagcat ctatgagaaa gaagaggccc cagatagcag ctgagcagag |
| 841 | taaagacaga gaaaatgggg caaaggaaag ctgggtgaat aatgtcaaca aagatgcttt |
| 901 | ccttcctcca gagctggctg gcgttgtaga agagccagaa gaagatgcag caccagcaag |
| 961 | cccgagttcc agtgtggtaa atccagcttc cagtgtgatt gatatgtccc aggaaaacac |
| 1021 | aaggaaacca aatgtgtctc cagagaagag gaagaatccg tttaatagct ccaagttgcc |
| 1081 | agaaggtcac tcatcacaac aaactaaaaa tgaacagtca aaaaatggaa gaactggttt |
| 1141 | atttcagact tcaaaagagg atgaattgtc agagtcaaaa gaaaagtcaa ctgtcgcaga |
| 1201 | tacttcaatc caaaagttag agaaatcaaa gcagactttg ccaggccttt caaatgggtc |
| 1261 | ccaaatcaag gctccaatcc ccaaagccag gaagatgatc tacaaatcaa ctgatttaaa |
| 1321 | caaagatgat aaccagtctt ttcctagaca aaggacagac tccctgaaag cgagaggggc |
| 1381 | tccgagaggg atcctcaagc gcaactccag ttccagtagc acagactcag aaacccttcg |
| 1441 | ttataatcac aactttgaac ccaaaagcaa aattgtgtca cctggcctaa ccatccatga |
| 1501 | gagaatttct gagaaggagc attctttaga agacaactct tccccaaact ccctggagcc |
| 1561 | attaaagcat gtgagattct ctgcagtgaa ggatgagctt ccacagagtc ctgggctaat |
| 1621 | ccatggtcgg gaagtaggag aatttagtgt tttagaatct gacagattga aaaatggaat |
| 1681 | ggaagatgca ggggacacag aagagtttca gagtgaccct aagccttctc aatacagaaa |
| 1741 | gccttcgctt tttcatcaat caacctcaag cccatatgta tcaaaaagtg aaacacatca |
| 1801 | gccaatgact tctggttctt ttccaattaa tgggctgcat tctcattcag aagttttaac |
| 1861 | tgcaagacca cagtctatgg agaattcacc aaccatcaat gaacccaaag ataaatcatc |
| 1921 | agaattaaca aggcttgaat ctgtattacc cagaagccct gctgatgaac tgtctcattg |
| 1981 | tgttgagcct gagccatctc aggtgccagg tggcagttct agagaccgtc agcaaggttc |
| 2041 | agaagaagaa cccagtcctg ttttgaaaac tttggaaagg agtgccgcta ggaaaatgcc |
| 2101 | ttccaaaagt ctagaagaca tttcatcaga ttcatcaaat caagcaaaag tagataatca |
| 2161 | gccagaagaa ttagtgcgta gtgctgaaga tgtttccaca gtgcctacac aacctgataa |
| 2221 | tccattttct caccctgaca aactcaaaag gatgagcaag tctgttccag catttctcca |
| 2281 | agatgagagt gatgacagag aaacagatac agcatcagaa agcagttacc agctcagcag |
| 2341 | acacaagaag agcccgagct ctttaaccaa tcttagcagc tcctctggca tgacgtcctt |
| 2401 | gtcttctgtg agtggcagtg tgatgagtgt ttatagtgga gactttggca atctggaagt |
| 2461 | taaaggaaat attcagtttg caattgaata tgtggagtca ctgaaggagt tgcatgtttt |
| 2521 | tgtggcccag tgtaaggact tagcagcagc ggatgtaaaa aaacagcgtt cagacccata |
| 2581 | tgtaaaggcc tatttgctac cagacaaagg caaaatgggc aagaagaaaa cactcgtagt |
| 2641 | gaagaaaacc ttgaatcctg tgtataacga aatactgcgg tataaaattg aaaaacaaat |
| 2701 | cttaaagaca cagaaattga acctgtccat ttggcatcgg gatacattta agcgcaatag |
| 2761 | tttcctaggg gaggtggaac ttgatttgga aacatgggac tgggataaca aacagaataa |
| 2821 | acaattgaga tggtaccctc tgaagcggaa gacagcacca gttgcccttg aagcagaaaa |
| 2881 | cagaggtgaa atgaaactag ctctccagta tgtcccagag ccagtccctg gtaaaaagct |
| 2941 | tcctacaact ggagaagtgc acatctgggt gaaggaatgc cttgatctac cactgctaag |
| 3001 | gggaagtcat ctaaattctt ttgttaaatg taccatcctt ccagatacaa gtaggaaaag |
| 3061 | tcgccagaag acaagagctg tagggaaaac caccaaccct atcttcaacc acactatggt |
| 3121 | gtatgatggg ttcaggcctg aagatctgat ggaagcctgt gtagagctta ctgtctggga |
| 3181 | ccattacaaa ttaaccaacc aatttttggg aggtcttcgt attggctttg gaacaggtaa |
| 3241 | aagttatggg actgaagtgg actggatgga ctctacttca gaggaagttg ctctctggga |
| 3301 | gaagatggta aactccccca atacttggat tgaagcaaca ctgcctctca gaatgctttt |
| 3361 | gattgccaag atttccaaat gagcccaaat tccactggct cctccactga aaactactaa |
| 3421 | accggtggaa tctgatcttg aaaatctgag taggtggaca aatatcctca ctttctatct |
| 3481 | attgcaccta aggaatacta cacagcatgt aaaagtcaat ctgcatgtgc ttctttgatt |
| 3541 | acaaggccca agggatttaa atataacaaa atgtgtaatt tgtgactcta atattaaata |
| 3601 | agatatttga acaagctagg aaaattgaat ttctgctgct gcttcaaaga aaaagctgcc |
| 3661 | ccagagcatt aaacatgggg tattgttaag aagcaaaatg ttcttgtttg ccatcatgtg |
| 3721 | tttcacacca caattctgtg ccacagttaa gagggtctgg tacccttgca ggacctttgt |
| 3781 | aggttgtggg aaaaagtcgc agaaagatac tcaaagtgga gcagggaatg gagacagaca |
| 3841 | tcagtgatga taaaaaaaaa aatggacctt aagaaactat ttactctgta atctctaata |
| 3901 | aaatatggaa ttccatatta gggcaatgag actgaaacta ctggtgtttt tctgccttga |
| 3961 | gaaaacaaac agttaaaaca agcctcaaat gtattttagt gccacccact ggccataggt |
| 4021 | acaattcagt tgttggcttg ttttgactta attctaaaat aggtctcaag cctgtatttt |
| 4081 | tatgagttta tttttttaaa accctgcata tatatgattg tttttcttat aactttacta |
| 4141 | tatgaaagca gcataagagt agtcacaaac atgtcttgca acaaagtttt aattagaatg |
| 4201 | taagttgctc agttatactg ttcttcttat gtatgtaaaa ttttcgtatt ttgtaaaaac |
| 4261 | ccttagaata aattatcatc tgatttaaat tgtattagaa aattagcgtg acttctcatt |
| 4321 | ttaaataaaa tattttagga attctaaa |

An amino acid sequence for the protein (SRR1-like protein) encoded by the SRRD gene that is a regulator of T cell exhaustion/dysfunction resistance is available from the UniPROT database at accession no. Q9UH36, cDNA NM_001013694.3 with gene location at Chr 22.

| (SEQ ID NO: 103) | |
|---|---|
| MAAAAAAALESWQAAAPRKRRSAARRPRRREAAPRGREAAPRGREAAPRGPEAEFESDSGVVLRRIWEAEKDLFISD | |
| FWSSALETINRCLTKHLEQLKAPVGTLSDIFGNLHLDSLPEESDVATDSIPREILVTGTCHLKCVCYGIGNFATCIV | |
| ARNQLTFLLLLLEKCQIPRSHCWVYDPLFSQLEIEVLNTLGVTVLSENEEGKRSIRGEPTIFYMLHCGTALYNNLLW | |
| SNWSVDALSKMVIIGNSFKGLEERLLARILQKNYPYIAKILKGLEELEFPQTSQYMDIFNDTSVHWFPVQKLEQLSI | |
| DIWEFREEPDYQDCEDLEIIRNKREDPSATD | |
| (SEQ ID NO: 104) | |
| 1 | gacgtcagag accaatggct gcggccgcag ctgcggcgct ggaatcctgg caggcggcgg |
| 61 | ctccgcggaa gaggcgctcc gcggctcgac ggccgcggcg gagggaggcg gcgccccggg |
| 121 | ggagagaggc ggcgccccgg gggagagagg cggcgccccg gggccccgag gcggagttcg |
| 181 | agtctgacag cggagtcgtg cttcgtcgca tctgggaggc tgagaaggac ctgtttatct |
| 241 | ctgatttctg gagttcagca ctagaaacca tcaatagatg tctcacaaaa catctggaac |
| 301 | aactgaaggc ccctgtgggg actctttcag acatctttgg aaacctgcat cttgactcat |
| 361 | tgccagagga gtcagatgtg gccactgatt ctatcccaag agagatcttg gtcacaggaa |
| 421 | cctgccattt gaagtgtgtg tgttacggca ttgggaactt tgccacctgc atcgtagcta |
| 481 | gaaaccagct aacgtttttg ctgcttttgt tggaaaagtg ccagattccc agaagtcact |
| 541 | gttgggtata tgaccctctg tttagccaac ttgaaattga agtccttaac acccttggtg |
| 601 | tgactgttct cagtgagaac gaggaaggga aacggagtat tcgcggggag cctaccatct |
| 661 | tttacatgct ccattgtggg acggccttgt acaacaatct tttatggagt aactggtcag |
| 721 | tagatgccct ttctaagatg gtcatcattg ggaacagttt caaaggactt gaggagaggt |
| 781 | tgttggcaag gattctgcag aaaaattatc cctacattgc aaagatttta aaaggactgg |
| 841 | aggagcttga gtttcctcag acttcacaat acatggacat atttaatgat acctctgtcc |
| 901 | actggttccc tgtgcaaaag ctagaacagc tctccataga tatttgggag tttcgggaag |
| 961 | aaccagatta tcaggactgt gaggaccttg aaatcatcag gaacaagaga gaagatcctt |
| 1021 | ctgctactga ctgaactcgt tgtgaggtac tcagtgttgg ctgaggtaga agctgccacc |
| 1081 | agagactaaa gggaaggctg ctatggagga actacagaga actcctttgc caggaaagaa |
| 1141 | catcaacttg gctgtcctgt tttgaggacg ataccccaca tgaggacttg gtataaagat |
| 1201 | tcctgcccta cgtggcattg tcccatttta catccttccc tcatgacctg gcctgatgtg |
| 1261 | gagtagctcc tgagtaaaga agttaccctt ttgaaggtga tctaaaaata ctgtttattt |
| 1321 | acagtacatc cctcttaggg gcaagtctct gactggttct ggacctgcca cagttcactt |
| 1381 | ggccatgtcg atcaggctcz gcagtgaggt gggcacccac gtcttctcgc cctggacaaa |
| 1441 | gaccactccc cggtcgatgt agatcacaat gcggccaaag gtgtagagct gcttcccttc |
| 1501 | gtgtcgcttc ccaatgacgg gcatgaagac aatgctgtgc tcctcagcct tggtctcaat |
| 1561 | gaggtcctta aagttcatgg gcacagagct agcggccacg ccaatgcccc tctgagccat |
| 1621 | gttctcagcc tcccgcctct cctgcatggc ctcgtactgg aagtccttcc tccgctccgt |
| 1681 | gtgggtgaga taggcaatgt tctcccgtgc tcctggctgc atgtaggcac ctaagataca |
| 1741 | ggaggacagg gcggtgagga gaggtgtttc caggtgtatg gaagttgaca ctgtggcttg |
| 1801 | gcccaggtct tgggtgtgcc agagtgcttg tgactcactg aagggcagct ctgcctcaaa |
| 1861 | gatacaactt tggaggaagt ttagacgact ggccagtatc acataaaaac tgttcagaag |
| 1921 | gttcctggct agtatctagt aaccagatta ttaatattca acattttaaa atgaagtatc |
| 1981 | tgcaagggat ttcttccttc agagctggaa acattaacag agagtcctca gccactcttc |
| 2041 | tgttcccacc ttgctctgta gagtttccat ttcccaccac caaagaggca gcttttagta |
| 2101 | ccttgaaaac atagggccca tactggcttt atttttaacc tacccacaca gggcccccat |
| 2161 | cctgaaagaa gttcacatat atttagggag ctgaaatgag tgtttaaagg ataataccaa |
| 2221 | gaccataaat gctaagtgct aattgagggg tatattcagg gaacagtttt ggaaacatga |
| 2281 | ggccaagggt agctaaggaa gagtttttag aaaaaacttg ataattacag aagcagagaa |
| 2341 | gtatatttgc cttattactt aatctctgtt ttcacagaat aggctatttt tttttttttt |
| 2401 | tttttgagac cgagtctcgc tcttgtcacc taggctggag tgcagtgctt gcaatctcgg |
| 2461 | ctcactacaa cctctgcctc ccaggtttaa gcgattcttc tgcctcagcc tcctaagtag |
| 2521 | ctggcattac aggcatgtgc caccgcgccc ggctaatttt tgcattttta gtagagacgg |
| 2581 | ggtttcacca tgttggtcag gctggtctcg aactcctgac ctcgtgatct acccgcctcg |
| 2641 | gcctcccaaa gtgctaggat tacaggcgtg agccaccgtg cctggcctag gctattaaat |
| 2701 | aacaagaatt aaagactcag gaaatagaaa ttccagttct aactgccacc ctttaactat |
| 2761 | gtgaccttga acaggctctc agcctcgctg ggcttatctg taaaacacag actagacgat |
| 2821 | cccatttcaa tgctgcgaat cggctaatga gcaacactgg agcacaggac tggttgaaat |
| 2881 | aggctcccaa atgtagaaac ataaatatac tctcacttac ttatgatcta ggcagaatga |
| 2941 | cttacctaga acttgggaac agatatagta cacgaacagt gaactgcctg tttttgacaa |
| 3001 | cacagacacc acctattgaa ggctatacac aagcatgaac tcactctccc ttttagccta |
| 3061 | taaagtagat cttactgttt aacaggtaaa atggggctga ttaaggctga gcaatcacca |
| 3121 | agtgtcagac cgcgtgccaa actccacact gtaagatgcg tcacctaagc agcatgctca |
| 3181 | ggcatgaatg agccttaaaa ggccacacag cacagtggtt aagagggcgg ggcctggggt |
| 3241 | tagactgaca tcatctgaat ccagcttaag tcagtctcca ctcagggaag atgcccctct |
| 3301 | cagtcatggt agacctgggc agtgggtgcc agctgaccat gtaccccagt cagcagggtg |
| 3361 | agagtctgtt gctatgtgca aaagtgtgac ctaaggttta ggctgcctac aggaaccaga |
| 3421 | aaactctgat tctgtgtcat ttacatgtgt aaaatctgaa acttggggcc aggacatcca |
| 3481 | aaatgggaat cctcacttac caacgttgga ggacaccgcc cggttcatga tatcaagtgc |
| 3541 | ctcattaaat ttgtccttga cagatggatg tgccagcact tggtctgaga acatcgactt |
| 3601 | ccaacccagg taccacttgg tgatctcctc ataatttggg ctgttactga gccaagagca |
| 3661 | cagcacctgc caaaaagaaa aattacttgc atccatcgtg gcaacaaatg aaggcaagat |
| 3721 | ttctgaagtg gccatttgtt ttgttttgat cctggtccta caggctagtg acactacttt |
| 3781 | acagggattt atagtagcta aacagtctag cacttatgaa tatggatttt ggagtcagcc |
| 3841 | tggtagctaa agtgcccttg catgtaaact ctctgagcct cagcttccat gtctacacaa |
| 3901 | cagggatacc atatccccta ccccataggg ttgctgagag gagctgagat aaagcaaata |
| 3961 | aagtgcttgg aacagcttct gatacatggc aaatgtccaa taaatggtgc ccactatgaa |

An amino acid sequence for the protein (Spindlin-1) encoded by the SPIN1 gene that is a regulator of T cell exhaustion/dysfunction resistance is available from the UniPROT database at 55 accession no. Q9Y657 or accession number NP_006708. cDNA NM_006717 with gene location at Chr 9: 88.39-88.48 Mb.

| (SEQ ID NO: 105) | |
|---|---|
| MKTPFGKTPGQRSRADAGHAGVSANMMKKRTSHKKHRSSVGPSK | |
| PVSQPRRNIVGCRIQHGWKEGNGPVTQWKGTVLDQVPVNPSLYLIKYDGFDCVYGLEL | |
| NKDERVSALEVLPDRVATSRISDAHLADTMIGKAVEHMFETEDGSKDEWRGMVLARAP | |
| VMNTWFYITYEKDPVLYMYQLLDDYKEGDLRIMPDSNDSPPAEREPGEVVDSLVGKQV | |
| EYAKEDGSKRTGMVIHQVEAKPSVYFIKFDDDFHIYVYDLVKTS | |
| (SEQ ID NO: 106) | |
| 1 | gaggcgccgt ctggctcagg ctggggccgg cgggagcgcg aacgagcgac ggcggaaccc |
| 61 | gtgggcctgt ggactgcagc ctcggcggtc agcagagtgc ttgtgatttc acgtattttt |
| 121 | gctgaactcg taaaagagac acttggatgg tggattaacc agaacactaa cattctgtga |
| 181 | aaagtttcaa attggagaat attgaatttt caccctagtc cagcagctcc gctgctcact |
| 241 | taaatacaga tgaatgaaga ccccattcgg aaagacacct ggccagcggt ccagagctga |
| 301 | tgcaggccat gctggagtat ctgccaacat gatgaagaag aggacatccc acaaaaaaca |
| 361 | tcggagcagt gtgggtccga gcaaacctgt ttcccagccc cggcggaaca tcgtaggctg |
| 421 | caggattcag catgggtgga aagaggggaa tggccctgtt acccagtgga aaggaaccgt |
| 481 | tctggaccag gtgcctgtaa atccttcttt gtatcttata aaatacgatg gatttgactg |
| 541 | tgtttatgga ctagaactta ataaagatga aagagtttct gcgcttgaag tcctccctga |
| 601 | tagagttgcg acatctcgaa tcagcgatgc acacttggca gacacaatga ttggcaaagc |
| 661 | agtggaacat atgtttgaga cagaggatgg ttctaaagat gagtggaggg gaatggtctt |
| 721 | agcacgtgca cctgtcatga acacatggtt ttacattacc tatgagaaag accctgtctt |
| 781 | gtacatgtac caactcttag atgattacaa agaaggcgac cttcgcatta tgcctgattc |
| 841 | caatgattca cctccagcag aaagggaacc aggagaagtt gtggacagcc tggtaggcaa |
| 901 | acaagtggaa tatgccaaag aagatggctc gaaaaggact ggcatggtca ttcatcaagt |
| 961 | agaagccaag ccctccgtct atttcatcaa gtttgatgat gatttccata tttatgtcta |
| 1021 | cgatttggtg aaaacatcct agatgtcatc acaaactctg ccaaatttgt ggaactatga |
| 1081 | aatgtattat ttgtagacat aaagacttga ttgccttcca gtttaatgaa agcttaaatg |
| 1141 | tccctgcgaa cccacaatct ctgccagcag aactggtttt gttctgaata gtacagattg |
| 1201 | atgtgaacac aaagcatttt gtgtaaggag aacccctttc ttttaaaaga agtctgtcta |
| 1261 | tttcgagggg agttacaggc aagtttggta aaagttaagc tagtatcata gtcatttaaa |
| 1321 | attgtaatag atcttaacca ttttccccct caccctaact ctcttattct gccgccacaa |
| 1381 | tgcaagcata gtttgatgtt ttcgttattg ccttttttga gatgtatgta tctgtatcta |
| 1441 | cctatatcta tatgtgtgta tacatatata atatatacac acagatacaa gtgtacacac |
| 1501 | acacaccaca caccacacac accccactgg cagtctttgt ggattaggat gggggtgaca |
| 1561 | taattttctt gagtttaaca ggagtgcttt acccaagtca gtcatggata tgatatcact |
| 1621 | gctctttatt taaaagtaaa ctttgggcac aggaaccata tgggaacatt cagcttgttt |
| 1681 | aaaaaaaaaa aatcagaagt tcagagcacc ttttcaatct ggagccctta aaccataagt |
| 1741 | ccaaatatga attatttgtg aacagttaat gacccatatt agaattttac aatctggtga |
| 1801 | acaatctggg taaaaggcta cctttaattc taagcttcat ttttttttgg gggggggtta |
| 1861 | cattttaatc ataaattctt ttttgcaaag cccaggttct tcttccacac agtgtaatgt |
| 1921 | aggtgtattt tggacagcac atatggtcca ttgtgtacaa gtcagtaatc tggatttcag |
| 1981 | tacatggatt taaaaggcaa caatccatca ctggcttgtg tgtttttggt taagtttctg |
| 2041 | gaagaataca atgcttttat attggaagta taagttttga gtggcattgt tgccttctaa |
| 2101 | caagctctct gggactttta aagttttatt actattattg ctaatgtatg attatggtca |
| 2161 | gagactttca acccacccct tcctgcctac ccctggcttt tcctcacaaa gaatgtctag |
| 2221 | acaaacagca gcttagtctc ctgacacctc ttgtggggtg catgttcctg ctccaggtat |
| 2281 | gctgttcatg tcgattcatt ccattcagtg gccccttggg gtttgcatgc cagtgaagcg |
| 2341 | ttaccataga agagccagcc gtttgtgttc gcttggtagg tgtaagtaat agcagccctt |
| 2401 | agccacggag tctttatgtg gtttattcct gcaaggttgt tttaaactga aatagaactg |
| 2461 | ctagtgtgat tggttgatca gcaaaacagc tgctctcatt ttgttttgga ggaaccctgg |
| 2521 | tggtttgaca gccaggagaa aggccatatg tgaaaccagt ctcttgtgga gattggaaat |
| 2581 | cctccctgat atttgggcag agcctaaacg tgcatgcttg tcactcaaac accagaagtt |
| 2641 | tgaatcagag gtctgagtga cctcttttgt ggctgttctt gatggtttcc aggcctcgtt |
| 2701 | atgcatggtt tgcttgatgc ccattttttg tgccttgtgc tgttggatgg taacgaccac |
| 2761 | tcaccatgta aacacagtac ctcagttttc tgtccacagc tgcagtcgct ctgtccctcg |
| 2821 | tgcttcactg tgggcagcca gggacaagct tctgaaagca gtgtgatacc caagtcatag |
| 2881 | gtgcttctcg tatgtgaaca ggtgtcattc tacagatctg ttatgtgttt tcctgttgac |
| 2941 | tcttaagagt ccctgcatgt aaatctcaaa gctgagcctg gctcccagag tccagactga |
| 3001 | cattacagcg caagagtttg caagtgtgtc cagcatgcac caactgtgca gaacttggct |
| 3061 | cggtaacttt gcgtggaaaa ataaagtaca ttttgaagta tcttgagggt tgggtcaatt |
| 3121 | gagacatttc tagcattact taatgacttg cattgtggtt tttctgcaag caactttaat |
| 3181 | gacttttttt ataccacatg gtctcccagt ttctagatga atgcaacatg atgatggtga |
| 3241 | tgatgacgat gagtttaatc attgttcatt tattgccttt agggctgagg gaaagggaag |
| 3301 | gtttgttttt ttttctcccc attttccccc attctgtctt tcttttggtg acttacacca |
| 3361 | catgtaatga cgctatgact aattctgctc ccaagccctt gtatcttggg cttcatttta |
| 3421 | ggctcatgtg tcagatctgc atgcattgct tgcatttttc tggtatctga atgttggttc |
| 3481 | cttgttccag gaattcaaca ttaatttcca aaagtatcat gggacttgtg acaatacaag |
| 3541 | acatgaatct atgtataaaa tttatcggcc tttctcattt acctgctcta gtattattgt |
| 3601 | attgtgtgtg cgtgcgtgtg tgatgtcagg ctgccacgta aaacttcaga gaaaaatctt |
| 3661 | aaaagcagac catccttttt tgcatgctct attctaagta gaatgttcaa tgtaactgac |
| 3721 | taaaattgca tgttaaagat atttaggttt ttttgttttc tttattttta tttgttttca |
| 3781 | gtttcctgta tatttgctta ctgtgccgtt ttagtggttt taggataaaa atgcactggt |
| 3841 | gaagcaaatg tagtgccaac agaaggtgat tttccagttg taaatgtcat gcagcatttg |
| 3901 | aagggactgt gttttcttaa aaaaaaatca cagttacttc taaaccagat ttcatttctt |
| 3961 | ttattgtttt atgtgccaaa ccacgaagtg cattgggctt caatctctga acactgtaga |
| 4021 | cccattagaa gactgttccg attgttacaa attgtagtgc ctgaaaacac tcttaagctg |
| 4081 | attgtcttaa caaaatgaaa gttctccaaa gacaaaacag aacaattatt ataacaaaat |
| 4141 | aattatggtt gaaatgtctg tggttccttg gaaatgctgc gctctttgtg tttttccatc |
| 4201 | attagtgcag ttggaatgaa tgtgtatagg tcagaggtct tcgtgttcac attttaaaat |
| 4261 | taggtaaatg acctcatctt tcaagcttga attcattttt aattttaatt ttattttata |
| 4321 | caatgtgtag acagttccct gttctctgca tttagaagta tacacaatat aaatctgtta |
| 4381 | attctgtaag taatttttat aattatgatg taactctatc ttatccttaa aacatttaaa |
| 4441 | ataaaccctt tatgtgcaa |

An amino acid sequence for the protein (Ubiquitin-conjugating enzyme E2 E1) encoded by the UBE2E1 gene that is a regulator of T cell exhaustion/dysfunction resistance is available from the UniPROT database at accession no. P51965 or accession number NP 00118940, NP 003332, NP_872607 or NP_003332.1, cDNA NM_001202476, NM_003341 or NM_182666 with gene location at Chr 3: 23.81-23.89 Mb.

| (SEQ ID NO: 107) | |
|---|---|
| MSDDDSRASTSSSSSSSSNQQTEKETNTPKKKESKVSMSKNSKL | |
| LSTSAKRIQKELADITLDPPPNCSAGPKGDNIYEWRSTILGPPGSVYEGGVFFLDITF | |
| TPEYPFKPPKVTFRTRIYHCNINSQGVICLDILKDNWSPALTISKVLLSICSLLTDCN | |
| PADPLVGSIATQYMTNRAEHDRMARQWTKRYAT | |
| (SEQ ID NO: 108) | |
| 1 | attgcctgtt taatagttgc tgttgctgca cttccgcttc tctcccagcg agagagagac |
| 61 | acgagtggcc aggcccagcc gcagccgcag cagcagccgc cgcggcggca cggaggagcc |
| 121 | agacacaaag agaggggctg tttgcggggt ggggtggggg gttcgctatg tcggatgacg |
| 181 | attcgagggc cagcaccagc tcctcctcat cttcgtcttc caaccagcaa accgagaaag |
| 241 | aaacaaacac ccccaagaag aaggagagta aagtcagcat gagcaaaaac tccaaactcc |
| 301 | tctccaccag cgccaagaga attcagaagg agctggcgga catcacttta gaccctccac |
| 361 | ctaattgcag tgctggtccc aaaggcgata acatctatga atggagatca accattctag |
| 421 | ggcctccagg atccgtgtat gagggtggtg tattctttct cgatatcact tttacaccag |
| 481 | aatatccctt caagcctcca aaggttacat ttcggacaag aatctatcat tgtaatatta |
| 541 | acagtcaagg tgttatttgc ttggacatat tgaaagataa ttggagtcca gcactaacca |
| 601 | tttctaaagt cctcctttct atctgctcac ttcttacaga ctgtaatcct gccgacccct |
| 661 | tggtgggaag tattgccact cagtatatga ccaacagagc agaacatgac agaatggcca |
| 721 | gacagtggac caagagatac gctacataaa ttggggtttc acaattctta cattatttgt |
| 781 | ctgtcacaga agagagctgc ttatgatttt gaaggggtca gggagggtgg gagttggtaa |
| 841 | agagtagggt atttctataa cagatattat tcagtcttat ttcctaagat tttgttgtaa |
| 901 | cttaaggtat cttgctacag tagacagaat tggtaatagc aacttttaaa attgtcatta |
| 961 | gttctgcaat attagctgaa atgtagtaca gaaaagaatg tacatttaga catttgggtt |
| 1021 | cagttgcttg tagtctgtaa atttaaaaca gcttaatttg gtacaggtta cacatatggc |
| 1081 | catttatgta aagtccctct aagactacat actttttgtt taaaacaaaa ttggaatttg |
| 1141 | ttttcccttc ttggaaggga acattgatat ttaacagagt ttttagagat tgtcatctca |
| 1201 | tatatataaa atggacacgt ggctataaaa caccatataa gagatgagta gtgcgtttta |
| 1261 | ttttatatgc caatctactt tctttaaaaa aggtctgaat caggacttgt gaaaacctgt |
| 1321 | agtgaaatac cttaagctgt taactaactg taaggcgtgg aataggagtt gctcagtgga |
| 1381 | ttggttctat gttgtggact acttaagtct gcatttgtta ctgtgctaat aaacaatatt |
| 1441 | aaaaaccacc taataaacac tgctgtgttc atttactttt cttttgcctt ttggttgcca |
| 1501 | ttcgcagatt cttctgaaat cgataggtat ctgcttctaa aacaagctaa aaataatgca |
| 1561 | tatttagtag gtattagtta gttacctata ttaggatctg tactttacac agctgtagat |
| 1621 | gaggtatcct agaaaaggtt aaataatcta ccccatttag gcttcaaaga cgaaccctac |
| 1681 | tgcatctttt taaaagcatt ttctgacttg cagadgctgt agtagcaagt acatgagaaa |
| 1741 | tgggttccgt catggataaa ttggtaccct gccttagggg gca |

An amino acid sequence for the protein encoded by the ARHGAP5 gene that is a regulator of T cell exhaustion/dysfunction resistance is available from the UniPROT database at accession no. Q13017, cDNA NM_001030055, with gene location at Chr 14: 32076114-32159728.

(SEQ ID NO: 116)
GAGGAAGAGAGGCGAGCGGAGAGTGGAGGAGGAGGCGGCGGCGGCGGGAGCGGTCCCCAGGAATGTCGCTGCC
GCCGCCACCGCCGGGGCCGCTGCCGTTGAGGAGGAGACGGAGGAGACCGACGTTGTTAGGAAGATGATCCCTA
TGATCTTGAAGATGTTTCTGCACAGAAATGAGGGAAATACAAAGAACCAAATACAGTTCTGAAATTTGGGATC
TGTATTTTGAGATGATTTTATTTTCAGAATGAGAAGCATATCTGGTTACCTTTATGAATGTAGAGACATGAGA
AGAGAGTTATGATGGCAAAAAACAAAGAGCCTCGTCCCCCATCCTATACCATCAGTATAGTTGGACTCTCTGG
GACTGAAAAAGACAAAGGTAACTGTGGAGTTGGAAAGTCTTGTTTGTGCAATAGATTTGTACGCTCAAAAGCA
GATGAATATTATCCAGAGCATACTTCTGTGCTTAGCACCATTGACTTTGGAGGACGAGTAGTAAACAATGATC
ACTTTTTGTACTGGGGTGACATAATACAAAATAGTGAAGATGGAGTAGAATGCAAAATTCATGTCATTGAACA
AACAGAGTTCATTGATGACCAGACTTTCTTGCCTCATCGGAGTACGAATTTGCAACCATATATAAAACGTGCA
GCTGCATCTAAATTGCAGTCAGCAGAAAAACTAATGTACATTTGCACTGATCAGCTAGGCTTAGAACAAGACT
TTGAACAGAAGCAAATGCCTGAAGGGAAGCTCAACGTAGATGGATTTTTATTATGCATTGATGTAAGTCAAGG
ATGCAATAGGAAGTTTGATGATCAACTTAAATTTGTGAATAACCTTTTTGTCCAGTTATCAAAATCAAAAAAA
CCTGTAATAATAGCAGCAACTAAATGTGATGAATGCGTGGATCATTATCTTAGAGAAGTTCAGGCATTTGCTT
CAAATAAAAAGAACCTTCTTGTAGTGGAAACATCAGCACGATTTAATGTCAACATTGAAACATGTTTTACTGC
ACTGGTACAAATGTTGGATAAAACTCGTAGCAAGCCTAAAATTATTCCCTATTTGGATGCTTATAAAACACAG
AGACAACTTGTTGTCACAGCAACAGATAAGTTTGAAAAACTTGTGCAGACTGTGAGAGATTATCATGCAACTT
GGAAAACTGTTAGTAATAAATTAAAAAATCATCCTGATTATGAAGAATACATCAACTTAGAGGGAACAAGAAA
GGCCAGAAATACATTCTCAAAACATATAGAACAACTTAAACAGGAACATATAAGAAAAAGGAGAGAAGAGTAT
ATAAATACTTTACCAAGAGCTTTTAACACTCTTTTGCCAAATCTAGAAGAGATTGAACATTTGAATTGGTCAG
AAGCTTTGAAGTTAATGGAAAAGAGAGCAGATTTCCAGTTATGTTTTGTGGTGCTAGAAAAAACTCCTTGGGA
TGAAACTGACCATATAGACAAAATTAATGATAGGCGGATTCCATTTGACCTCCTGAGCACTTTAGAAGCTGAA
AAAGTCTATCAGAACCATGTACAGCATCTGATATCCGAGAAGAGGAGGGTGGAAATGAAGGAAAAATTCAAAA
AGACTTTGGAAAAAATTCAATTCATTTCACCAGGGCAGCCATGGGAGGAAGTTATGTGCTTTGTTATGGAGGA
TGAAGCCTACAAATATATCACTGAGGCTGATAGCAAAGAGGTATATGGTAGGCATCAGCGAGAAATAGTTGAA
AAAGCCAAAGAAGAGTTTCAAGAAATGCTTTTTGAGCATTCTGAACTTTTTTATGATTTAGATCTTAATGCAA
CACCTAGTTCAGATAAAATGAGTGAAATTCATACAGTTCTGAGTGAAGAACCTAGATATAAAGCTTTACAGAA
ACTTGCACCTGATAGGGAATCCCTTCTACTTAAGCATATAGGATTTGTTTATCATCCCACTAAAGAAACATGT
CTTAGTGGCCAAAATTGTACAGACATTAAAGTGGAGCAGTTACTTGCTAGTAGTCTTTTACAGTTGGATCATG
GCCGCTTAAGATTATATCACGATAGTACCAATATAGATAAAGTTAACCTTTTTATTTTAGGGAAGGATGGCCT
TGCCCAAGAACTAGCAAATGAGATAAGGACACAATCCACTGATGATGAGTATGCCTTAGATGGAAAAATTTAT
GAACTTGATCTTCGGCCGGTTGATGCCAAATCGCCTTACTTTTTGAGTCAGTTATGGACTGCCGCCTTTAAAC
CACATGGGTGCTTCTGTGTATTTAATTCCATTGAGTCATTGAGTTTTATTGGGGAATTTATTGGGAAAATAAG
AACTGAAGCTTCTCAGATCAGAAAAGATAAATACATGGCTAATCTTCCATTTACATTAATTCTGGCTAATCAG
AGAGATTCCATTAGTAAGAATCTACCAATTCTCAGGCACCAAGGGCAGCAGTTGGCAAACAAGTTGCAATGTC
CTTTTGTAGATGTACCTGCTGGTACATATCCTCGTAAATTTAATGAAACCCAAATAAAGCAAGCTCTCAGAGG
AGTATTGGAATCAGTTAAACACAATTTGGATGTGGTGAGCCCAATTCCTGCCAATAAGGACTTATCAGAAGCT
GACTTGAGAATTGTCATGTGCGCCATGTGTGGAGATCCATTTAGTGTGGATCTTATTCTTTCACCCTTCCTTG
ATTCTCATTCTTGCAGTGCTGCTCAAGCTGGACAGAATAATTCCCTAATGCTTGATAAAATCATTGGTGAAAA
AAGGAGGCGAATACAGATCACAATATTATCATACCACTCTTCAATTGGAGTAAGAAAAGATGAACTAGTTCAT
GGGTATATATTAGTTTACTCTGCAAAACGGAAAGCTTCGATGGGAATGCTTCGAGCATTTCTATCAGAAGTTC
AAGACACCATTCCTGTACAGCTGGTGGCAGTTACTGACAGCCAAGCAGATTTTTTTGAAAATGAGGCTATCAA
AGAGTTAATGACTGAAGGAGAACACATTGCAACTGAGATCACTGCTAAATTTACAGCACTGTATTCTTTATCT
CAGTATCATCGGCAAACTGAGGTCTTTACTCTGTTTTTTAGTGATGTTCTAGAGAAAAAAAATATGATAGAAA
ATTCTTATTTGTCTGATAATACAAGGGAATCAACCCATCAAAGTGAAGATGTTTTTCTACCATCTCCCAGAGA
CTGTTTTCCCTATAATAACTACCCTGATTCAGATGATGACACAGAAGCACCACCTCCTTATAGTCCAATTGGG
GATGATGTACAGTTGCTTCCAACACCTAGTGACCGTTCCAGATATAGATTAGATTTGGAAGGAAATGAGTATC
CTATTCATAGTACCCCAAACTGTCATGACCATGAACGCAACCATAAAGTGCCTCCACCTATTAAACCTAAACC
AGTTGTACCTAAGACAAATGTGAAAAAACTCGATCCAAACCTTTTAAAAACAATTGAAGCTGGTATTGGTAAA
AATCCAAGAAAGCAGACTTCCCGGGTGCCTTTGGCACATCCTGAAGATATGGATCCTTCAGATAACTATGCGG
AACCCATTGATACAATTTTCAAACAGAAGGGCTATTCTGATGAGATTTATGTTGTCCCAGATGATAGTCAAAA
TCGTATTAAAATTCGAAACTCATTTGTAAATAACACCCAAGGAGATGAAGAAAATGGGTTTTCTGATAGAACC
TCAAAAAGTCATGGGGAACGGAGGCCTTCAAAATACAAATATAAATCTAAAACCTTGTTTAGTAAAGCCAAGT
CATACTATAGAAGAACACATTCAGATGCCAGTGATGATGAGGCTTTCACCACTTCTAAAACAAAAAGAAAAGG
AAGACATCGTGGAAGTGAAGAAGATCCACTTCTTTCTCCTGTTGAAACTTGGAAAGGTGGTATTGATAATCCT
GCAATCACTTCTGACCAGGAGTTAGATGATAAGAAGATGAAGAAGAAAACCCACAAAGTGAAAGAAGATAAAA
AGCAGAAAAAGAAAACTAAGAACTTCAATCCACCAACACGTAGAAATTGGGAAAGTAATTACTTTGGGATGCC
CCTCCAGGATCTGGTTACAGCTGAGAAGCCCATACCACTATTTGTTGAGAAATGTGTGGAATTTATTGAAGAT
ACAGGGTTATGTACCGAAGGACTCTACCGTGTCAGCGGGAATAAAACTGACCAAGACAATATTCAAAAGCAGT
TTGATCAAGATCATAATATCAATCTAGTGTCAATGGAAGTAACAGTAAATGCTGTAGCTGGAGCCCTTAAAGC
TTTCTTTGCAGATCTGCCAGATCCTTTAATTCCATATTCTCTTCATCCAGAACTATTGGAAGCAGCAAAAATC
CCGGATAAAACAGAACGTCTTCATGCCTTGAAAGAAATTGTTAAGAAATTTCATCCTGTAAACTATGATGTAT
TCAGATACGTGATAACACATCTAAACAGGGTTAGTCAGCAACATAAAATCAACCTAATGACAGCAGACAACTT
ATCCATCTGTTTTTGGCCAACCTTGATGAGACCTGATTTTGAAAATCGAGAGTTTCTGTCTACTACTAAGATT
CATCAATCTGTTGTTGAAACATTCATTCAGCAGTGTCAGTTTTTCTTTTACAATGGAGAAATTGTAGAAACGA
CAAACATTGTGGCTCCTCCACCACCTTCAAACCCAGGACAGTTGGTGGAACCAATGGTGCCACTTCAGTTGCC
GCCACCATTGCAACCTCAGCTGATACAACCACAATTACAAACGGATCCTCTTGGTATTATATGAGTAGGAAGT
GATTGCAAACAGGCTGGATTTGGACAAAAAGCAAATCTAGACATGCATGTTTCAGGGTTCAGTAGTATACTTC
ATGTTTCATACAGATAATTCACATTCAAAATTACATTTTCTCTTTGAACTAGATGGTATTCCTTATTCACTTA
CATTACAAATCTAAGACCATGTGATAAGCATGACTGGAGAGGTTTAATTTTTATAAACAAAAATAGCTATAAA
GTACAAAGCTGCTGCTGCATGCAACCTTATTGCAATCAGTATATCATTCCTGTGGCAATTTCTGTCACCTTAT
ATTGTGAATAAAATTTTTCTATAGAAATTAAATGATTTAAAAACTCACCTATATGAAACATTTAATGCTTTTC
AGCCTGCTTTCTGGCTGATTTTGTTATTTGATGTGCTAATTTGGGCAACTTAATTTACATTCTGGCAGTCGGT
GTAGATAACTAAAAGCCCAGTTAAGTATTTTATAATTTCAGGCTACTGAGGCCATGCTTGGGATGTTGTTTGA
AAGAAAGAAAAAATACACTTGACATATTTCACATTTCTGTACCTTCATCTTTACTTCCAAGTAAACCCGTGGA
TGATTTGATGAGGGATAAATGAACCTATTTCTTTTACACACATACCAAGGACATGCTTGTGGCTAAAGTGAGT
TGATAATGTTGTGCAAAGGATAGTTGTCACCAACTCATTTCTTTATGGTCCATAATGAAATAAAAATTTTGTA
TACTGTTAATTCTGTAAACAGATGCATGTTCAAAAGATCTATGATGGTCTTGTAATCTTAATCTAATATATTT
TAGATATTTTAATTTTTTCCCTCTTGGGGAACACATTTAGTATAGTGTAGAAAATACTTCCATGACATTTTCA
TATAAGGTTATATAACTTTTCATACATAAACATGAAATTTGTTGTAGAAAATTCTTTAAACCAAACATTTAAA
TCTAGGACTTCAATTTAATTTGTTCCTTGAATCTATTTTTATGTGGCCCTTAAAAAATATCCAAAAAACCCAT
TGCTAATATAGCAATAAAAATACTTTGGGTACTGACAGACTCTTTGGAGTGTTTATATTACAAATTTGTATTC
ATATTCTTTTCTGTGATGTGTTGTACTAAAATCCAAAATGGCTTTTGCACCATTTTTAAGCCAATTTTTTCCT
TTGATGTTGGTACCAGAATTACTATAAGTGACTGCTGCTTTTGGGGGTAAACATTTTGTTAGTGAAGATAAAA
CCAGAACACTAAATTATGGATAAAATTTTCAGAATAGGTGGCACAGGTAAATTTCACTAGGTTATATTTTGTG
TAGTAAAGAAAAAAATTATTTGGTCAATGTTATCTTAATTCATACTACAATTTAAGATTATCTTATGTGTATT
ATAGTAAATAGATGATTTTCAGATTCAAGGCTCCTAAGAGTTTGATTTGCTCTGTTTTTTCCTAAAATAAATA
TTGTCTCTCCCAACTGTTAAGTTCTAGGTATTGTACTTCCAATTTTAACTTCAGAACCAAGATGTTGGCATGA
ACCAGGCTGCTGTTGAAGTACATGTATATTATAAATTATCTTATTTGTGTTATACTCTTACATGTTATCTTTT
CTAAGAAAACAAAGTCCCTATTATTCCTATTGCAAAGCACACAGGAATTAAGAAAGTACAGTAATTTTTAAAA
AAAAATCCGGTAAATGTAGTATTCTTAACCTGTTCTATATTACTTATACCTATTGTCTATATAGCTTTAATTT
ATAGTTGTCAGTTTAACTATTGGCATGTCTGGCAAAGAAAATTAAACTTTAAGAGTTTTATAAACTGTTTCTA
GGTTGCTAAAGAATTTATTTTTCTACTATATATGGTATAGACAAAGCATCAAACTATGTACAGGAAAAAAGCC
TGACTATTTCTATTTGGAAGTAGGCTGAAAAGAGAATTTTCAAAACTGTTCGTGTCTTCAGTTCATTCTGTCA
TAACTTTGCTATTGTAATATGTGAATACCAGTTTATTTAAGCTGTTCTCTTTTATACTGTATTAATTTAATGT
TCATCTGCGTTTAGTACCATTTTTGTTATTAAAACTGGCATTTACCGTTTTTCACATTAACCCACCTTGCACC
TTCCCCCAAACTTATCTCCACTTTTCTATGCATTCTATCATTGATTTGACACACTTCATAGTGAGTCATTTAA
ATACTCTACGTTTGGTTCAATTAACCAGTAGGTTACAGTTATTGAAAATTAAAGTACAGTTTAAAGCTCAGTC
TGTTACACTGAATTGATTGTGTTTGTTTTTGCCAAGGGTTTAGATATGCTTTTAAATATTAGAAACATCTAAG
AACAGAATAACATAATTAAACTTTTTTCTGGTAAGTTACTGGAAGGTTTCACTGTTTAGGGACCTATCATATG
AGACTTCTTAAAGGATTAAAAGAATAGGATAGTCTCATAATTGTGAGTAAACATCAAGGCATTATATTTTACA
ATACTGAATAAAATTTCATCTACACACATGTTGCCATTGTTTCATTTAAGGTTCAGTGCTTATAGTTAACTAC
AATATTGGACCTAACAGGATCTAGATTAGCAATATAAAGAAGCATAGTGGTACTCTGTTTCACACTTTCAGTA
GATTTATTAGAAGTCAAATTCTATTCAACAGACACTTATTAGGATATACAACTAATTTAAGAATAAAATTCCA
GGCACAATATATTTTTTTTAAATGGTATTTGTTAGTAGTGCTTCTTCCCCTTAACATTTACAGTGTAAATACT
GCAGGTAACCGCAATCTAAGTTAGCCAAAAAGCAGCTTTTTTTCCCATACTGTATGTAAATAATGTAGACCTG
GGTTTTTTTGTTTATTTGGGTTTGTTTTTTTTTTTGAGGTACTGGAATCTAATTAATATCTCTTAGGTATCAA
CAAAAGGGAACAATTGGAATGAGAATTTAGGCCTTAGCTTCCATGGTGATTTTTAGTTTTTTATACAGTAATA
ATTGTGATGCTATTTGTCAACTGGATATAAATACACATATAATTTTAAAAAGTCAAAAGTGCTTTTGTTTCTT
TGTTTAATGTAATTTTTGTGCTTCACCTACAGGATGCTGCAGTAAATTAAATATCAGTGAAGCTTCTGATGTA
TAAAGAATGCTATGAATAAAACATTAAGAAGCTGTGTAATTTTAAGTTATAGTTGCCTCTATTTTTACCATTT
CATTGGTAAAAATTAGCTAATTTTTTTCAAGTGAAATGAAAAATAAAAATATAAATTTATCAATATGATGGAA
ATCTTATTAAGGAGATGTATTATTGAATTTTCACTGTACCTGAAAAGGAGATTCAAAATTTTTTCTGGGGATG
TATATAGGTGAAAATTTGATTTTTTAAATTATCAGGAAAACAAGATAATGCACAGATTTCTAAGACTAAGATC
TTACCTGGATGTGATTTTTGAGCTGTGGCTAGACATTCTTTAGAGCCACTGGAAATATTTTGAAAACTATTCT
AGTTATAGCAGAGCTGCTAATATTAACGAATATATTTGTGTCTTCATGGTTTGTGACTATTAGGCCAAATTTT
GTGGTATATGTTGTCAGTCTGGATCTGGTGAGGTCTGTTCAACATGAATCTTTGTGTTATCTTGAATTTAGTA
GTTTCAAGGTACTTAAATTCTTAACAGTTTCTAATTTGTTTCAATACATATGGGACATGGTTGATTTTTTTAC
TGTATTAGAACTCTTGGAAGTTCTTAGCCTTTTCAGGTTATGAAATACCTGAAAGTAAAATTTTCTAAGATTT
AATAAGGGAAGATACTATTCAAATCATTTTCTTAGGATAGCATCTTTACATACAATGAGAGGATTGTACAAGC
ATTAATCTCATATTCCAACATCCAGTTACTTGATGTGATCCAAGTACCCTGGTCTTTTTGAAGCAGTTAAAAT
CTAATTAATTAACTTTGGGAGTCTTCACTATTCAATTGATCCTCATCATTGTCCTATTTGCATGACTCCATTT
TTTCCTCCACTATATGAGTTTTCTTTGTCAGGGGGAGAGGAGTGGGAAGAGTCACAGAATCTCATATTCACAT
CTTAATTAAATTGTGTGAAATTAGTCTTTTGTGGAAATTCTGTAGGCAGTATGATTTTGAAAAGCTAACCAAT
GATAATTAGCATTTTAGTTAATACTAAATGCATAAAATTATAACCCTTGAAATTAATTTGGTGCTGGCAGTTC
TGGTTTAGTCATTTTTACCAGTAGTTAGTAGTATTAAGACCTGCAGTATATGCACTTTTTGAGTAGCTGTCAA
ATAATTGTAGTTGAGAAACAACTTGTTTATTCTCACAATTCAGATTTTCTATTCAGTTTTGTCTCAAATAGTA
AGTTATTGTGAACAATTTAATAACGGCCCTCCTGTTCTAGTTTGCCTAATATTTTAGTTAAGATTTAGTGTTT
TAACCTATTTTTTTAAGTTTATTTTTTGTATTAGATTTTATTTGAATAAGTTATGTGGGTTTAGTAATTGACC
TATTTATTCATTGCTTCACTAATTCATCCAGATTAGTTTTAAGTGTGTATATGTATTTGCTCACCAGATCATT
TTCTTGGGACCTTGAACTGTGAATGTTTTGTCCTAACCATTTAATATTTTCTAGGTACTTGCTGCAAGTTCTT
GAACTATTTTACCAGCTTTAACTTTGGGGCTCTTAGTTTCTTTTCTCCAGATTCTTGTTATTTTATTTTATCC
AAATAAATATTTAGGTGTTCTAAGA
(SEQ ID NO: 117)
MMAKNKEPRPPSYTISIVGLSGTEKDKGNCGVGKSCLCNRFVRSKADEYYPEHTSVLSTIDFGGRVVNNDHFL
YWGDIIQNSFDGVFCKIHVIFQTFFIDDQTFLPHRSTNLQPYIKRAAASKLQSAFKLMYICTDQLGLFQDFFQ
KQMPEGKLNVDGFLLCIDVSQGCNRKFDDQLKFVNNLFVQLSKSKKPVIIAATKCDECVDHYLREVQAFASNK
KNLLVVETSARFNVNIETCFTALVQMLDKTRSKPKIIPYLDAYKTQRQLVVTATDKFEKLVQTVRDYHATWKT
VSNKLKNHPDYEEYINLEGTRKARNTFSKHIEQLKQEHIRKRREEYINTLPRAFNTLLPNLEEIEHLNWSEAL
KLMEKRADFQLCFVVLEKTPWDETDHIDKINDRRIPFDLLSTLEAEKVYQNHVQHLISEKRRVEMKEKFKKTL
EKIQFISPGQPWEEVMCFVMEDEAYKYITEADSKEVYGRHQREIVEKAKEEFQEMLFEHSELFYDLDLNATPS
SDKMSEIHTVLSEEPRYKALQKLAPDRESLLLKHIGFVYHPTKETCLSGQNCTDIKVEQLLASSLLQLDHGRL
RLYHDSTNIDKVNLFILGKDGLAQELANEIRTQSTDDEYALDGKIYELDLRPVDAKSPYFLSQLWTAAFKPHG
CFCVFNSIESLSFIGEFIGKIRTEASQIRKDKYMANLPFTLILANQRDSISKNLPILRHQGQQLANKLQCPFV
DVPAGTYPRKFNETQIKQALRGVLESVKHNLDVVSPIPANKDLSEADLRIVMCAMCGDPFSVDLILSPFLDSH
SCSAAQAGQNNSLMLDKIIGEKRRRIQITILSYHSSIGVRKDELVHGYILVYSAKRKASMGMLRAFLSEVQDT
IPVQLVAVTDSQADFFENEAIKELMTEGEHIATEITAKFTALYSLSQYHRQTEVFTLFFSDVLEKKNMIENSY
LSDNTRESTHQSEDVFLPSPRDCFPYNNYPDSDDDTEAPPPYSPIGDDVQLLPTPSDRSRYRLDLEGNEYPIH
STPNCHDHERNHKVPPPIKPKPVVPKTNVKKLDPNLLKTIEAGIGKNPRKQTSRVPLAHPEDMDPSDNYAEPI
DTIFKQKGYSDEIYVVPDDSQNRIKIRNSFVNNTQGDEENGFSDRTSKSHGERRPSKYKYKSKTLFSKAKSYY
RRTHSDASDDEAFTTSKTKRKGRHRGSEEDPLLSPVETWKGGIDNPAITSDQELDDKKMKKKTHKVKEDKKQK
KKTKNFNPPTRRNWESNYFGMPLQDLVTAEKPIPLFVEKCVEFIEDTGLCTEGLYRVSGNKTDQDNIQKQFDQ
DHNINLVSMEVTVNAVAGALKAFFADLPDPLIPYSLHPELLEAAKIPDKTERLHALKEIVKKFHPVNYDVFRY
VITHLNRVSQQHKINLMTADNLSICFWPTLMRPDFENREFLSTTKIHQSVVETFIQQCQFFFYNGEIVETTNI
VAPPPPSNPGQLVEPMVPLQLPPPLQPQLIQPQLQTDPLGII

An amino acid sequence for the protein encoded by the ARIH1 gene that is a regulator of T cell exhaustion/dysfunction resistance is available from the UniPROT database at accession no. Q9Y4X5, cDNA NM_005744, with gene location at Chr 15: 72474330-72602987.

(SEQ ID NO: 118)
AGTAGCGATAGCAGCGGCCGTGGAGGTGGCGTTGGGGACTGTTTTCTCTCGGAGGCCGGAGCGGAGCCGCGTC
TGACTGAGGCGGGCAGCAAGCGGCCCCCTCGCTCCCTCCCTCCCTCCTCCGCGCCCTCCCCGCCGCCACCAGC
CGTCAAACGCCAACCGCCGCTCCTGGGGAGGAGCCGCGGCTCGCGGGGCCGGAGCCAGGCCTGCGTCCGGACA
TCAGCCGGAGCCGGAGCGAGAGCCGGGGCCTCGGCGTCCCCGCCCTCTCCCCGCCTCGGCCAGCGTCCGCCGG
GCCCCCGCGCGTCGCGCCATGGACTCGGACGAGGGCTACAACTACGAGTTCGACGAGGACGAGGAGTGCAGTG
AGGAGGACAGCGGCGCCGAGGAGGAGGAGGACGAAGACGACGACGAGCCGGACGATGATACCCTGGATCTGGG
CGAGGTGGAGCTGGTGGAGCCCGGGCTGGGCGTCGGCGGGGAGCGGGACGGACTGCTGTGCGGGGAGACGGGC
GGTGGCGGCGGCAGCGCTCTGGGGCCCGGCGGTGGCGGCGGCGGCGGCGGCGGCGGTGGTGGTGGCGGGCCGG
GGCATGAGCAGGAGGAGGATTACCGCTACGAGGTGCTCACGGCCGAGCAGATTCTACAACACATGGTGGAATG
TATCCGGGAGGTCAACGAGGTCATCCAGAATCCAGCAACTATCACAAGAATACTCCTTAGCCACTTCAATTGG
GATAAAGAGAAGCTAATGGAAAGGTACTTTGATGGAAACCTGGAGAAGCTCTTTGCTGAGTGTCATGTAATTA
ATCCAAGTAAAAAGTCTCGAACACGCCAGATGAATACAAGGTCATCAGCACAGGATATGCCTTGTCAGATCTG
CTACTTGAACTACCCTAACTCGTATTTCACTGGCCTTGAATGTGGACATAAGTTTTGTATGCAGTGCTGGAGT
GAATATTTAACTACCAAAATAATGGAAGAAGGCATGGGTCAGACTATTTCGTGTCCTGCTCATGGTTGTGATA
TCTTAGTGGATGACAACACAGTTATGCGCCTGATCACAGATTCAAAAGTTAAATTAAAGTATCAGCATTTAAT
AACAAATAGCTTTGTAGAGTGCAATCGACTGTTAAAGTGGTGTCCTGCCCCAGATTGCCACCATGTTGTTAAA
GTCCAATATCCTGATGCTAAACCTGTTCGCTGCAAATGTGGGCGCCAATTTTGCTTTAACTGTGGAGAAAATT
GGCATGATCCTGTTAAATGTAAGTGGTTAAAGAAATGGATTAAAAAGTGTGATGATGACAGTGAAACCTCCAA
TTGGATTGCAGCCAACACAAAGGAATGTCCCAAATGCCATGTCACAATTGAGAAGGATGGTGGTTGTAATCAC
ATGGTCTGTCGTAACCAGAATTGTAAAGCAGAGTTTTGCTGGGTGTGTCTTGGCCCATGGGAACCACATGGAT
CTGCCTGGTACAACTGTAACCGCTATAATGAGGATGATGCAAAGGCAGCAAGAGATGCACAGGAGCGATCTAG
GGCAGCCCTGCAGAGGTACCTGTTCTACTGTAATCGCTATATGAACCACATGCAGAGCCTGCGCTTTGAGCAC
AAACTATATGCTCAGGTGAAACAGAAAATGGAGGAGATGCAGCAGCACAACATGTCCTGGATTGAGGTGCAGT
TCCTGAAGAAGGCAGTTGATGTCCTCTGCCAGTGTCGTGCCACACTCATGTACACTTATGTCTTCGCTTTCTA
CCTCAAAAAGAATAACCAGTCCATTATCTTTGAGAATAACCAAGCAGATCTAGAGAATGCCACAGAGGTGCTC
TCGGGCTACCTTGAACGAGATATTTCCCAAGATTCTCTGCAGGATATAAAGCAGAAAGTACAAGACAAGTACA
GATACTGTGAGAGTCGACGAAGGGTTTTGTTACAGCATGTGCATGAAGGCTATGAAAAAGATCTGTGGGAGTA
CATTGAGGACTGAGAATGGCCCTGCATAAAATGAACTCTGAAAACTTTACCATCTAGAGTGCTCATGCAATTA
AAACAAAACAAACACAAACAAGGAGGCACTAAGCCTATTCTGACACCACTGGTCTGTAGTACCAGAATTGTTT
TGTTAATGGAAAGTTTAAGTAAATTATATTGTAATAAAAAGGTAGATAAACCATTGTACAACAGTATTCTAGG
CCACCAACAAAAGTGTGACAGACACACTAAAAGCCCTCCAACTTTAACTTGTAACGTAGCTTCATTCTCAAAG
CTGACTCCTTTTTTTTCTTTTTCCTTTTCCTGAGTGTAGTACAGTTAAAATTTCAAACAGCTCCTTGACACTG
CTTTTCATGTTCAAACCAGCCATTTTGTTGTACTTTGGTAAAGGACCTCTTCCCCTTCCTCCCCTACACATAC
AGATACACCCACACACAGACTGACTCTCTTTCTCTCATACCCCAAGGTCATGAGTGAATGATGCTTAGTTCCT
TGTAAAGAAAATCTTGGGATGGGGAAAGGGGTAGGCAGCAAGAGGATTCAACAAACGAAAAACATAAAAACTT
TGTATATGACTTTTAAAACAAGAGGACAACACAGTATTTTTCAAAATTGTATATAGCGCATATGCATGGACAA
AGCAAGCGTGGCACGTGTTTGCATAATGTTTAATTACAAAAAAATATTTATTCTTTAAAAATCTTCAAGATTA
TGTCTATTTGCTGTGCATTTTCTTTCAGTTTGCTTATCTTTCCCGGGTTGGGGTTGGGATAAAGGTGTGTCGG
TTTAGCACCTCTGGAAGACCTATCTAGAGCTCTTTCACTTTCCTGAGGTTATTTTGCCCTTTCTGGTGTTGGT
ATGTCTGTTGCCGGCCATGGGCCTCATGCCTTGAATTCCTGCTCTTGATCAGGGACAAGGGAGGTCAAGCTCT
GACTAATGCCATGACCTGATTAAGGGGTACAGCAGGGAGTTTTGTTGCTACAGCTCATGAATTAACCTGTCCC
AACCTAATCCCCCTCCATGGCATCATGCCTCTACCCAAGCCTTTGTGTGCCCATGTTATGCACACAGCTGTAG
GCATTCTTAAGTCCCCTGTCGCATCCAGTGGAAGCATTTTAAAATTTCTTTTACTTTTTGGTTTTCCCTTAAT
TGCTGCTTTTCAGATTTTAGTTATGGCTCGTCTGCTCACCCCTTCTCTACATTAGGGTGTCAAAGAGAATGTT
TTGCTTTAAATATAAATAGCCATTCATTTAGTCTCAGATTGTGAATTTAAAATGGTGGATACCGAAATTGCTT
GTGTGTGTTGCTGTGGGTTTGGTTTGAAGGCAAACACCCCTAGAACATGATATTCCCATCTAGTGCATTTAAA
TAGAAATCACTGAGTTTGCTGCTTTTTTATTGTCAGCAGATAGGAGAATTAATAATGCATTTTAGCTGTGATG
TCCATTTTTATGAAATTCCTACTAAGAGCTATGTTAAAAGTAAAGGATGGTGGTGGTTGTATTAACTATATAC
CTGTTTAGGCCATTCTGGCTGTGGTATTTTTCAATAGGTCAGCATCTGTAAATCTGTCAGTTTTATACAGGAG
TGCAGAGTGAACTAGGCAACTAGATTAAGAGGTCTAAATATGAAATACCAGTTGAGGCTGAGGACCTCTTCGT
CTTCCTTTAAATGTCTTTTGCCTAGGGAGTGTTTACCATTTGTGAGGCAGCTTTGTCTGCTCTTACACTGTAC
ATCCTATTACTCCATTGGGAAGTAGGTTCACTTTCCTCTGGCCTTTTGCCTAAGTTAGGCTTTGCTGAATCAA
CCCTACTTTTCCTTTTAGAAAAGGTTGTTACAGGAGATTTACTGGCAACTGTTCTTTTCCCATCAAAAATCAG
TGAATGTTTGCTGAGTATAAATGCTGCTTCCTTAAACCACTTGTCGCTTTAGGATCAACTTTACCTGTACCTT
TTCTCCTTTCCTCCCTTGCCACCTCAGGTGCAAATCTGAACTCAGTGTCTGCTTCTTCCATTTTCTCGTCTCT
CTCCCCTCTTCCCCCATTATCCATATGACATTATTTTACTTCAAATGACAGCATCAATCTTAAAAAGATATAC
ATTAAAACTAAGGAGTTTTTTTAAAGAAAGCCTGAATAAGTTCCTTTCCCTGGTAACTTTGAAAAGCAGTCAG
AGTTGCTATATAGATATATGTGGCTCCTTTAAAATGCTTTGTGTATGTGTGGTGTTTAAAAAAAAAAAATCTT
ACCAGTTAACTTTGCAGTGTCTAGGTTTGAGTGTCATAAATCCACGTGTTCCTGTTGCAACAAATACCCAAAA
ATTGTGTGTGCACTTCCTAATACCAGTCTTCACCCATGGAGGAACAGTGCTTTTTAGAGATGCTTTCTATTTC
AATGTTGGCATACTGCCTGAGGGTATTGCAGTTGTGGGTGCATTCCCTAATTTGTATGATCAAGATGAACTGG
CCCTTTTCTACTTCCAAGCTTTTAACAGATACCACCATATTTGACAGAATTCCCAGAGTGAATTGCTTGTGTT
ATTAGTAGATTCAGTGCCCCCAGCTGGGATAGGCAAGCCATGACAGCTTCCCTGTTTCACCTACAGAAGTCTT
ATCTGAGGGATCTATTCACAGTAAGCACCAAGGTCTCCATGTCTTGAGGTCAGTTTCATTGTCTTTTGAAAAG
TGCATGCTTCATTTGAACAATTCATTCAGCAGCAGATGGACTTTCAGTGATTTAAAATAAAATTTTGATCCAA
AGCTCAGGACACAAACCACAGTGGTAAAATTGAGTAGCATATAATATCAGACTAAATTATCTGTAATTTTCCA
CAACCCAGATTGTATGTGTTTTATGTGTGTTTAAATAAATATGTTAGATACACGTGTATACATACACCCATAT
ACAACAGATCCAAGACTGGCTGACTTCATTTGAAATGGTTGAATCTGCTGTGTAATAAAGTGGTTCAACCATG
ATTAGGAACTGAAATTTAGTAGAAGAGGGAAAAGGAGTTAATGTAACAAATTATTTTAGCTACAAACCCCGGT
AATAGAGCACTTGGGGGATGGGATGGGGTGGGTTGGTGAGACAATCAGAATGGTAAATTGATTAAATGCTCCT
AACCCTGTAATTTTGTGCATAGAGCACCCTATGCTGTGGAAATAACTGTTCTTAGATTTCATTGTAACTGGAC
TGTTCAGGTTGCCCAGAGGGAAAGAACATTCCTAATTCTAATAAAATAAACTTTTATTTTGTTATTCCATTAG
TTACTTATGTTTATTTTTATAGTTAAAAAATGACTAAAGGTGGCTAATAGCCACCCGTATTATGTGCCTTATT
CACTGTTCCCACTTAATATAAAATGTCAAAAATTGGTTGTTTCTTTGCAGGGAACTGTGAATGAGCCCATTCC
CCCAACATTTAACTTGGTTAATGAAGCTTTCAGAGCATTTTCTGAAAGATTTCCAATTTCTGCATCTCAGATA
TTGAATTTAAATTGTTTGTGTTGTAACAGTGATACAAAAATGTTCAATAGCTCTTTGAATGCCACTTTTTTTA
AAGAAAATACTTTAAAAGTCATTCTTAGCTAACCTCAAAACTCCTTTACATTACTTTCATTTTAGCTCACTCT
TAAAGCTGATACTGTTATATAGGGATGAAGGGAGAGTTTTCTTAAAGAAGGTCCCAAAGTTGAAGATCGTTTG
TCATTGATACTCTGGCCAAGTCCAGGTTTTAGGACAATTTAATTTACTCTTATTTGGATCTGTAATTATGGGA
GTTTATCACTTTTCCTCTTCAAAGCACTTCAACTTACTGTCTAAAACAAGTGGAGAAGAAATTGCCATTTTTT
ATAAAGGAGGAAGATAAGGCTCACTGAGGTTAAATAACTCCCTCAATTTTTCATTTCCTTCTACCTGAAACTT
AACATCATGCTACCTCTTTGTATCATATTTTGTTATTCTGGTCACAGAATGACCTAGTATTCTGTACCAGGGA
AGGTAGTTCTTAACTATATTGTACTTTTAAACCACATTAAAGACTATTATAAATGCCTATCACTGCTACTGTT
AGAGGTTGCTCTATACTATCTCTGATCTTTCATTTGTTGCAGTTTGCAACACAGTAGTTGAGAATAAGTGGTT
TAGTATGATTTGCTTAATAGTACCCAAGTAATTTGCAGTTGTTGGTTTAGTTATTCAAATGAATTTCCATTGT
AAAGAATTTTGGATAGTCTGCAGGAAATTGTTACAGGATGCACAGAACATCCCATATCCATTGGATCTCAGCC
TTTGAGGCTTTAAAAAGATGATAAGCAGTGAGTTTTATGAGAAAATTTTGGCTCCTTCAGAAACAGGAAGATA
AGTGGGCCCCTCAGATTTTTCATATATCTTTGGGGATAAAGTTGGTTATATCTGGTTTATTTTAGGGATTTTG
CTTCTGTAGTTACCCAAGATCAAATAGCCAACTCAGAAATACTCAGTTGGATATCAATCAAATAGTGCCTACC
AGGGGAGTAAACAGACAACTTTTAGGTTTTTAGTTTTTGCTTTTGTGTGGAGTTGAGTCCAATTTTTAAGGCA
ACTATTGTTGACCTTTCTCTAGGCGGTAGCATTTAGGATGGGAAAAAATAATATGAGTTATGTGCGAAAAGCA
GTTTTCAGGCTATCATTTAGGGCCACAGATATGCTTATTTAGGACTATTTTCAGGGTAGGAGCCAGTTAAAGT
TTGTATATCAGTCACACATTGATATGTAGCAGCTCTATCAATACCACAACAACCTTATTACCATAGTAATTCA
GGGGTCTCAAGTAATCCAGGAGTCTCAAACTGAAATGTCAACAAGAGCCAAGTAAATATTTTAGAAATAGAGT
ACCCTGGAGTAGATGGGCTCTGAGAGGAGCCTGTGAAATCCAGACTTCATGTGAATTCTCTTAATTAAATGTG
GGCCAAAGAAAACCCGGCCATGGGCAGTTTTTTTTTCCCAGCTTCTACTAGTCTTGGCTTGCAACAATGCCAC
TGTCCATCTGGCATTACCTAGACCAGTGATACTTAAACTTGACTGCGAATTGGAATCACTGAGAAGCTTTAAA
AAATATTAGTGCCTGAGTGTTACTTTATGGAGATTATGATTTAATTGCTTTGGGTAGGCATCAGTAATTTTTG
TAAAGCCCTTTACGCATTCTCACCCCTAATTCCAGCCGGTGATTCTCACATGCAGACTAGGTTGAGATCCTTG
ATCTAGACTGCCTTCTATAGTTGCAAAAAGAGAATATAGGCGAGCAACTCCAGATCATAAGGGACAAAATGAT
TCAAGGCAAATTGACTGGCTCAGAATAGGGCCTCACTGGAGGCCCTAGTTGAGTGGGTTTTGTGGAATGGTGC
CTCCCAAGGGTCTGGTGTGCAGTGCCCAATTTGGTTTTTTGTTGGTTTTCTGGTAAGTGGTCCACCTTGGAGA
AAATCACAAAACTAAAAGTCTTACCAAGAAGTAGAACTGACAAGCAGCCATGTTGATTGGCAGTACATTCTCT
TACCAAGGGGGAGAAATCTATCTAGGGAAAATAATACAGAGTCTGAGCAAAGGAAGTTGAGTGGTTATAAGCA
AAACTGTCATTAAAGCTAGTTGCGTCAAGTCTGCACAATTCCACGAGCACTTAAGAGTTTACAAGTTTCTCAT
GTCTATCATTCTTTTGATAATCTTGTATTTTTATTTCAAGTGTGGTATAGTAGTAAGAATATTGGTATTTCTC
AGCCATAGATTTCCCTTTTGTAAAATGGGAATACTAATAGGAACTGTAATTTGTTGAGTGCTTTTTGCCAGAT
GCCTTACTTTTGTCTGTATCTTCCAAGCAGCCCTAAAGCAAAATGGGCATTGTTACCCTATTTTTATGAGGAA
AGACACTCATATACTACTAACTTACTCAAAAGTTACACATTGAGTGTATAACAGGGCCATGCTTGTCTGATTC
CATATGTTCATTCTATTATGCTGCTGTTGTCTGTTACATAGGAATATTGTGAGGATCAAAGAACATGTGAAAA
CAAAACCTAGTTACAATAATAAGCACACCAGTGCTAACTTGGGCAAAAGATAAGGATACAGAGCTGTCTCAGA
GACTCCCAAAGGCAGGATGAGATTGGGAACTAGAAAACCATTTTGGACCCCTAAAGTGGTATTGTCTACTATC
TGTACATCATTCTCTTACAGCTCTTACTGCTGCTTTTCCTGTCAGTTACCCCATAGCTCCAGGTATTACATGT
TAACTGTTCCTGACACATGTAGACAGAACCAATCAGATCCATGGGGCAGGATCATCAATACAAACATGGGTGT
TGAGAGCTTAACTTAATCGGGAAGGAGATCTTGGAGATCATAGTTCGCTGGGTTCACATCATGGAAATCGTCT
TTGACAAAACTGAAATGCCCTGAAGTTTTAATTTTTTAAATACATGTAATGAATTGGTATATGGTAGATACTC
GTGGCTACTTAGTATTTACCAAATGCTTTGTTTCCATCTCGTTTATTTTTATTTTTTATTTTTTTAAGAGACA
GTCTTGCTATGTTGCCCAGGCTGGACTTGAACTCCTAGTCTCAGCCAACCCCTTCTGCCTCAGCCTCCTGAGT
AGCTGGGATCATAGGCATGTGCCACTGCTCCTAGCTCTTCCATCTCAGTCTTATGATGTAGTTACTATTTTCA
TTTTATAAATAAGAAAATAGAAGCTCAAATAAAATGGGTAGTTTCCTTGAGGTCATCCTGTTAGTAAGTGGTG
GAATGAAGTTACAAACCTAGGTTTTATTGGTCAGCACACACAGTGCCGGGCACTGGCAGTACAGCAGTGAACA
AAAGGTCTGTTGGTTTGTCTGAAGCTCTCAACCTTGATTCTGCATAGACTCGGTGGATTGCCCAAGGCCACTC
AACCAGTTAATATCAGAGCCAGCTCTGCTGTTTGACCAGTATATTATTTTCACCATGCTGTATCTGCTTCTCA
GTCACCAAAAGCACCAGCTAAAATTGTATATAGACTGATTAGGGGACTTCCAACCTAAGATTGTCCTGGAAAC
TTAATTCTTATCTTGGCTCATCTCTTCAAAACAGATGCCTCTTTCGTTGGTGACCTCAGATACACTGTTCAGA
AGTACTCAAAGTATTCCTTTTTTCTCACATTTGAGAGTCACTCTAGCATTTTAATGAGTTAAGCAAGCCAGCC
CTGCATTTCCAAACCCTTCCCAGAAGTCTAACCTTACTTTGATCTGATGACATGGGCTTTTTAAAAGATTACT
ATTTTTTTAGAGGTGGGGTCTCACTATGTTGCCCAGGCTGGAATATAATGGCTATTCACAGGCACAGTTAGGG
TGCACTACTGCCGTGAACTCCAGGACTCAAGCCATCTTCCTGCCACAGCCTCCTGAGTAACCTAGGACTATAG
GTGCATGCCACCATGCCCAGCTGACAATGGCTTTATATACATAATATTTCCTCAGCAACAATTTATGTCTTAT
TTGGCACCTGTAAAACCCTCTTCACTGATTATGCCTGTTTAAAGGTTTGGTATTTACTCCAAGTCACCTGCCT
TTAGCCATGGAGTATTCAAGTTTCTTTCAGAATGAGAATTTGGAGGGTGCTCATAGAAATATAAAACAAGGCT
GGGCATGGCGGCTTACACCTTGTAATCCCAGCACTTTAGGAGGCTGAGGCAGGTGGATCACGAGGTCGGGAGT
TCGAGACCAGCCTGGCCAACACAGTGAAACCTCATCTCTACTAAAAATACAAAAATTAGCCAGGCATGGTGGT
GCATGCCTGTAATTCCAGCTGCTCGGAAGGCTGAGGCAGGAGAATCGCTTGAACGCGGGAGGTGGAGTTTGCA
GTGAGCCAGGATCACACCACTGCACTCCAGCCTGGGCGACAGAGAGACTCTGTCTCAAAAAAAAAAAAAAAAA
AAAAAAAGAAGAAGAAGAAGAAATACAAAACCAACTCTTTATAGTAGACAGTTTATCCCCCAGAAAGCTTTTT
AGGCTGAAAACGTTTCTATAGATTTTGGATTTCCTTCTGATAGCTAAAATTTGTTTCTGTATAACAGAGTAGT
GCCCTATAAGCCAGTTCAGTGCACATTAAATGGCTGTTCTTAGAATTATGAATCTGAGGCTTATGCAGTGAGG
CTCTAGCTCTCTGGAGTCAGTTGGTCAAGTGGGATTCCTGAGGACTGAGCAAACACACACAGAGCAGGATTTT
GTCCTAAGTTTTCTTAGGGATCCTATCCAAAAACACCATTACTTGAATTTCCCAGCTTCCTTTTACAGGGAGA
CTTCCTTGCTATGTGTACTCCCACCTCGGTATGTCAAATCATTTTCTCAGAGGAAGTACAGCATAAAATAATA
ACCAGCATTTGTAGAGCACTTCTTGTGGGCCTTCTTGGGCAAATAGACATTCTCATGGAATGCTCTAAAATTG
TTAAGTTCTTGCACTTGCTCTCAAGTCATTTATAATCTAATTGCTGGTCTTCCTGGTATGAACAAAGATGTTT
GCTTGTTAATAAATTCCTACCACATACCCAGACTTGTGGTTTATTTCTATCTAACTAGGGAAAGCACTAAAGA
GTTTTCCAATAAGGAAGAATGATCTGAGTGTCTACCTGCTTTGGAAAAGATGTCTGCCATGGATAGGAAATGA
TTTTGTATCCCAAAATCCTAGTACTAATTTGGAGTGTTGTAAAGGTCAGGGCAGCAAATGCTAGAGAGTGGGA
TTAATGAAAAGTTATAGTGTTCTGCATGCCTTTGAATAATTAATTTTTACTCCATTTTTACTCGCTTGCAATA
AAGTAGGTTAAGTGCTGTACTTTAAGGAAGTCCATGTACAAGCCAGTGCCTTAACAATGGTCAAATGGTTGCT
CACTAACCAATGGAAATAAATGACTGTTGTTTCCACAAAATTGCTTTACAGATTCTAAATGCTATCTCTGGGA
CACTGGATTGTGTGCTCTTTAAGATAAATTCCATAATCAAGGGCCTGGCCCTGAATAGATTGGAGCCCAAAAA
TCTCACTGAAGCAAAGAGATAATAGGCCAGTAAAGAGTGGGAGACAAACACTCCTGGAATAGGAAGCCACTAC
TCACCTTTATTTTAAATTGATATATAGAAATTAATATTACAAAATGCACACATCTTAAGTGTGAAGTTTGATG
TTTTGACAGTTGTATACGTCTGTGTAATAACTACCAAAAGAAGACATAATATGTTACAATTAACCCAGAAAAT
TCTCAAGTACTTTTAAATCACCCCTGAGCACCTCCCCACTCCAGTGATCACTTCTTGATTTTTATCACTAAAG
ATTTGTTGTACCTGCTTTTGGATTACATATAAGTGGAATCATATAATAATGTACTCTTTTGCATCTGGCTTCT
TTCACTGATAATGCTTTAAAGACACGCATACTGTATGTATTAGGACATTGTTCTTTATTGCTGAGTAGTATTT
CATTCTATAAATATACCACAATTTGTTTATTCATTCTCCTATTGAAGGACCTTTAGGTTGTTTCCAGTTTGGG
GCAATTATGAATAAGGATGCTATCGATATTCTTACACAGCCTTTTGTTTTTGTTTTTTGTGGATATACATCTT
CACTTGTCTTGGATAAATAAGAGTAGAAAATTACTGGGTCATATGGTAAGTGCATGTTTATAAGAAACTGCCC
TCTTATTCTCCAAAGTGGTTTTACCATTTTGTACTCCTATCAGTGATGTATGAGAGTTACAGTTGCTTCACAT
GTCTGTCAACATTTACACTGTCACTGTTTTTTATATTAGCCATTTTAGCATATATGAAATAACTGATTTTAAT
TTACATTACATTTCCCTGATTAATGAGTTGAGCACTTTTTCATATGCTTATTTTTCATTTGTATATCTTTCAA
GAAGTGTCTTTCATGTCTTTTCTCATTTTAAAGTTGGGTGTCTTTTTATTGTAGGTGTTATTCTTTGTATTTT
GAATATAAATCCTTTGATAGATATGCTATGATTTTTTAAAGACTCTATGTATTCACTTATTGGGATCTTTCGA
TGAACAAATTTTTTTTATAAAATCCACTTTATCAGTTTTTTATTTTATGGTTTTATGACCTCAAAGTCACAAA
GAATATATTTTCTTCTAGAAGTTTTATGGTTTGGGATTTTATGTTTAGGTCTGTGATCCATCTCAAATTAATT
TTTGTGAACAGAATGAGGTTTGGTTTTTTTCCAACATGGATATACAGTTGTTCCAGCAACATTTACTTAAAAA
GACTTTTCTTTACCCATTAGATTGACTTGGTAGCTTGGTTAAAAATTGATCACGTGTGTGTGTATTTCTACAT
TTTCTGTTCTGTTCCATTAATCTATTTGTGTATCCTTACATCAGTACCACACTTTGGATTATTGAAACTTTCT
AGTAAGTCTTTAAATTAGTAAATCCTTCCTTTTTGTTATTTTTCAAGATCGCTTTGGTTATTGTCGATTCTTT
GGATTGTCATAAAAGTTTTACAATTAGCTCATCTTGTTTTTCAAACGTAGATGTGCTGGGTTAGCTGTGTTCA
TGCCACTGCATTCCAGCTTGGGTGACAGCAAGACCCTGTCAAAAAAAAAAAAGAAAAGAAAAAACTGCTAGAA
TCTTGATATGGGGTTTTGTTGAGTCTATAGGTCAATTTAGAATGAACATTTTAATAGTATTGAGTCAGCTAAT
TCATGAACATATTAAGTCTTGTCATTTATTTAGGTATTAAATTTTTTTCTCAGCAATGTTTTGTAGCTTCCCT
TGCGTAACACTTGCTTATCTTACATTAGACTTTTTCTTAAGCATTTGATTTTTAAAATATTTTTGATTTTCTT
TTCTTTTTCTTTTTTTCTTTTTTTTTTTTTGAGACGGAGTTTTGCTCTTGTTGGCTAGGCTGGAGTGCAGTGG
CGCGATCTTGGCTCACTGCAACCTCCGCCTCCCGGGTTCAAGCAATTCTCCTGCCTCAGCCTTTTGAGTAGCT
GGGATTATAGGCACACACCATCATGCCTGGCTAATTTTTGTATTTTTAGTACAGACGGGGTTTCACCATGTTG
GTCAGCCTGGTCTCGAGCTCCTGACCTCGTGATCCAACCACCTTGGCCTCCCAAAGTGCTGCCATTACAGGCG
TGAGCCACCGCGCCTGGCTAATTTTCATTTTTAAGTTGTTTGCTGCTAGTATCTAGAAATACAATTGATTTTT
TGTTTGTTTCTTTAGAGATGGGTCTCACTATGTTGCCCAGGTTGGTCTTAAACTCCTGAGCTCAAGGGATTCT
CATGCCTCAGCCACCCAAGTAGCTGGGACTTTATTCACGTGCCAGTGTGCCTGGCTGATTTTTGTATGCTGAT
CTTGAATCGTGCCACCTTGCTAAATTCATTTATTCTAGAAATAGTTCTTGTAGACTCCGTAGAGTTTTATATT
AAATAGATACACAACAGTGTAGTCTGCAAATAATGTTTTTACTTCTTTTTCTGATTTTATGCCTTTTCTTCTT
TTTTTTTTTTTTTTTTTTTTTTTTTGTCTTTCTCCACTGGCTACAATATCTAGTGCAATGTTTAACGTGTGGT
GAGAGCAGACACCTTGCCTTGTTTTCTTAATCTTAGGGATAAAGATTTTAATGTTTCATCACTAAGTATAATG
TTAACTGTAAGTTTTTCACAGACCAGCCCAAAGAAGTTCCTTTCTATTCCTAATTTGTTGATAATTTTTATTA
GGAAGGGTTATTAAATTTTGTGAGATGCGTTTTTAGCTTTTAATAAGATTATTATGTATTTCTCTTTTTATTT
TGTTAAGATGGTATGTTACAGGGGTTGGCAAACTGTGGCCCATGGGCCAGATCCAGCCCATGGACAGTTTTGT
TGTTGCTGTTTTAGAGACAGTCTCACTCTTTTGCTCAGGCTGCCAGGCTGGAGTGCAATGACATGATCATAGC
TCACTGCAGCCTTGAGCTCCTGGACTCAAGCGACCCTCCTGCCTCAGCCTCCTGAGTAGCTAGGAATACAGGT
GCATGTCACCACACCAGGCAAATTTAAGAAATTTTTTGTAGATCTTGCCGTGTTGCCCATGATGGTCTCAAAC
TCCTGGCCTCAAGCAATCCTCCTGCCTTGGCCTCCTACAGTGCTGGGATTAGAGGTGTGAGCCACCACACTTG
GCTCTCATGGACTGTTTTTGTTTTTGTTTTGAGACAGGGTCTCTGTCACGCAGGCTGGAGTGTGGTGGCATGA
ACGTGGCTCACTGCAGCCTCAACTTTCTTGGCTCCAGCAATCATCCTGCCTCAGCCTCCCGAGTAGCTGGGAC
CACAGGTGTGCGCTACCACAGCTGGCTAATTTTTCTTTTTCATTTTTGTAGACATGGAGTCTTGATATGTTGC
CCAGATTGATCTGGAACTGCTAGGCTCAAGTGATCTTCCTGCCTTGCACTCCCGAAGTGTTGGCATTACAGGC
ATGAGACACAGTGCCCAGCCTATTGCAGTGTTTTTTGTTTTTTCTGTTTTTTTTTTTTTTTTTTCATCTTTGT
TTTTGTTTTTTTGAGACTGAGTCTTGCTCTATCTCCCAGGCTGTGGTGCAGTGGTGCAATCTGCAACCTCCGC
CTCCCAGGTTTAAGCGATTCTCCTGCCTCAGCCTCCCAAGTAAGTGGGATTATAGGCACATGTCACCAAGCTC
AGCTAACTTTTATATTTTTAGTAGAAACGGGGTTTCACCATATTGGCCAGGCTGGTCTTGAACTCCTGGCCTC
AGGTGATCTGCCCACCTTGGCCTCCCAAAGTGCTGGGATTACAGGTCACTAGGCGTAGTAGGGCTACTACGCC
TGGCCCTACTGCAGTTTTTAATAAGAGATGAGTTCTCTCAGCTTTTGTCTATAAGAGAATGTCCTTACTTTTA
TTTTTGAGGGGTATTTTAGTTGGATATAGGACTCTATGTTGACCTTTTATTTCTTCTTTCAGTACTTTAAAGA
AGTTATTTTATTGTACTCAGGCCTCATTTGTTTCTGGTGAGAAGTCATTCATCATTTTAATTTTTATTTCCTT
TCCGTAATGTGTGGTTGTTCTCAATGGCTGCATCTGTGCATTATATTTACTTTTCAGTAGATTGACTAGGGAA
TGTCTAGGTATGTTTTTCTTTGTATAGAGTTTACTGAACTTCTTGGATCTGTGTTGATGTCTTTCATTAATAT
TGGTTCTTTCTTGGCCGTTATCCGTTTACATTTTTCTCTTGTCTCATACTCTCTTGCTTCTCCTCTTCTTTGG
AGATTCTAGTTACATGTATATTAGATGATTTGATATAAACCTACAGATCTCAGATGATTTTTCCCCTGCCCTG
TTTTCTTTTTGTTTTCAGTTTGGATACTTTCCATTTAACCATCTTTAAGTTTTTTGATGCTTTCCAATGTTAA
CCTGTTAAACTTGTTTATTGAATTCTCCATTTAAAAATTTTTATTTTTAGTTATTTATGGATACATAGTTGTA
CTGAATTATTCATTTCTAATATTTTTATTTCTAGCATTTCATTATATTTGTATAGTTACAATTTTCTGCTGAA
ATTTCCTATTTCTTCATGCATGTTTTCCACATTTTCCATTCTACTTTTTAGCATTTTAATCAGTTATTTTATT
GGGTTTTTTCTGTTTTTTCTTTCATTTTTAATCAGTTATTTTAAGGATCCAAACTGATAATTCAACATCTGGG
TCTTTTCTAGTTCTGCTTCTATTGAGCCCTTCCTCTCCCTTACCATGAATCTTATTTTCTTGCTTTTGCACGT
CTTGTATATTTGATTGGATGATGGACGTTTTATGTTGAAAAAAAAAAGAAATTGAAGAAAATGTTTACTTCCA
AAACAGAGCATGCCCTTTGTTTTTTTTTCAGGCCACTTGAATGGTGGCTCAATCTGATGTATATGAATTCTAG
ACTGGGGCTTCACTGCAGTTTTAGTTTGATTCAGTTTGCCTCTAGCTTCAAATATTTGAAGGAACGTAGAATC
AGAACTCTCATATCACCAGGGCCTGAGTTTTGGGCATTGGCAAGATTCCAGAGATCTGACTTTGCTTCATAGC
TAAGCCACCAGCTTTTATCAATGTGGGAGATCTTTCCCTACTTTACAACCTTGCTGCCAGGATTTTTTTTTTT
AATCAATGGGCTGGGGATAGGAGGATCTTTCTCAGCTCTCATGTGTGCCCTGCCCTTTTGTAGAGGAATGCCC
ACAATGCCTAGGGGATGGGTTTCTCAGCATTTCTGACCCCATTTAGGCTCCAAGAGTGCCTCTGTTGGATTTC
ACAACAATCCTGCCTCCCCTTTCATTTTTAAAGGGTATTTTGCCCTTGGGAGAAGTCAAGCTCTCTCAGCGTT
CCTTCTATTATCCACACCTTTAGTGTACTACACCTTCCTGTACCTGGTTATCATCTAAGGGGTAAGATTGGTG
GGAAGCCTTCCTCTGTGGATTTGGGTCATCTAGGTTCTAATGTCAAACCAGCCTCCAAGGGGTTGTGAAAATG
TTGTTAAATTTCAGCAGGTTTCTCTTTTCTCCCCCCTGTAGTATTCCTCTTCCTACCATAGTTCAACTAGGAA
TAACAACAGCTAGGGGGTCTCTTCTCTCCTATGAAGGACTTGGCACTTTCTGGAGTTTGGATCATTTAGGTTT
CTTTGGTTTTTGTTTTTATTTTGAGACAGGGTCTCACTCTTACCCAGCCTGGAGTACAGCCTGTAGTGATCTC
AGCTCACTACAACCTCTGCCTTCCTGGCTCAGTTGATCCTCCCACCTCAGCCTCCTGAGCAGCTGGGACTACA
GGCGTGTACCACCACACCCGGCTAATTTTTGTATTTTTTGTTGAGATAGGGTTTTACCATGTTGCCCAGGCTG
ATCTCAAACTTCTGAGCTCAAGCGATCTGCCTGCCTCAGCACCCCAAAGTCCTGGGGTTACAGGCATAAACCA
CTGCACCCAACCTAGATTTCTTTGTGTCCTTAGCTTTCTGATGAATTAAACATATGTTTTATTTTCTAGCTCA
TCTGGCCTTTTGTTGTTGTTTGCTAGAGTGGGAGGTATATTCCTTGTGACTTTGTTCATATCACTAGTGAATT
TCAGAAGTAGGAGTTTATTTTTTTAATAAAAAATTTTTTTAGGTCAGGCACAGTGTCTCATGCCTGTAATCCC
AGCACTTTGGGAGGCCGAGGCAGGTGGATCACCTGAGATCAGGAGTTTGAGACCAGCCTGGCCAACATAGTGA
AACCCCAACTCTACTAAAAATACTAAAATTAGCCAGGTGTGGTGGTGGGCGCCTGTAATCCCAGCTACTTGAG
AGACTGAGTCAGGAGATTTGCTTGAACCTGGGAGGCGGAGGTTGCAGTGAGCCGAGATTGTGCCACTGCACTC
CAGCCTGGGTGACAGAGCAAGACTCCCTCTCAAAAAAAAAAAAAAAAATTTTTTTTTAAACACGTCTCACTGC
CACCCAGGCTTGAGTGCCATGACATGATCATAGCTCACTGCTGGCTTGACCTTCTGGGCTCAAGTGATCCTCT
TGCCTCAGCCTCTTGAGTAGCTGGGACCACAGTCATGTGCCACCATGCCTGGCTTTTTTTTTTTTGTAGAGTT
TGGGTCTCGCTATGTTGCCTGTGATCCTCCCACCTTGGTTTTCCAAAGTGCTGGGATTAAAGGCATGAGCCAC
CATGCCTGGTTAGGAATGGGAGTTTAAACAATGGTTTTATTTACATATTTCATTAGCTAGTATTTTTTTCTTT
TTTGAGACAGAGTCTGGCTCTGTTGCCCAGGCTGGAGTGCAGTGGCACGATCTAGGCTCATTGCAACCTCTGC
CTCGTGATTCTCGTGACTCAGCCTCCTGAGTAGCTGGGATTACAGGCGCCCGCCACCACACCCAGCTAATTTT
TGTATTTTTAGTAGAGACAGGGTTTCACCATGTTGGCCAGGTTGGTCTCCAACTCCTGACCTCAGTTGATCCA
CCCGCCTCAGCCTCCCAAAGTGCTGGGATTACAGGTGTGAGCCATCGCTCCTGGCCTCATTAACTGTTTTCAA
AAGTAAAAGTACATGGATAATACAAAGTTAAATTTTGTAGAATCATAAAGAAAAGCAACACCTTCCCATCTTA
ACTCCCCTAGTGCCTCTACTCCCTCCACAGAGACAAATATCTTACCAGTTGGTTGTGTTTTCTTTTCCAAACT
GTATTTCTAAAATGTGTCAATATCTCTTCCTTTTATTTTTTATTTTTTGTAGAGACAGGGGTCTCACTGTGCT
GCCTAGGCTGGTCTCAAACTCCTGGCCTCCTACCTCGACTTCTCATAGTTCTGGCGTGAGCTACTGCACCTGG
CCTCCCCCTTATTATTTTTTTAAACATGAATGGAAGCATACTATATTTTCTACACTTTATTTGTTTTATGTAA
CTATTTTAACAACCTGAGGACTTTTACAGGCTGCATAATATGTCTTAATATGGATGTATCTTAGTTTATTTAA
ACAGTTACTGGTGATGGACATTTCCATGTTTCCAGTATTTTTTCTCACAAACAATGCAGTATTATGCCTTTGT
CCACATTCACCATTGATTGCTATTGAGTATATCTGTAGCATCAAGAATAGGCATACTGAAAATCCTCCATGAA
GTCCAGTGAAGCTTCATATTATTTTGAAGTGATATTTCCCAGTGTTGGCTGGACTCTCAAGCGTTTGTGGTAT
AGGTTTTGGGAGGACTGGACACTTCCAAAGAGAAATAAGATGTTACAGACTTGTCCAGCAGGTGGCATCAGAA
CAAAGCTGGCTATGGACCTTGTCAGTACAAATGATAAAACATTAAAAATGTAAAAACTCAACTGTAGCTCCTT
GTTTATACATATATTGGGGGTGTTGAAAAGTTACCGATGCCTGATTCCTCTTCTATGTCCTCAGCCCCCACCA
TAAAAATAGATAAATGCCATTTTTTTTGTATGCCCAGCACAGCTATATGATTTGAATAATCTTGATAAGCAGG
GTCACATTTGAGGAGATTTGCTAAGGGAAATGAAGATCCATCTGTGACACAAGAAACATGTTCTATGTGATTT
CTGGGATTTTCCTGTGAACATACTTCTTGTTTGTTTGTTTTACAGTGGATCTCACTCTGTCACCCAGGCTGGA
GTTCAGTGGTGCAATCATAGCTCACTGCAACCTTGAGCTCCTGGGCTCAGGAGAGCCTCTCTCCTCAGTCTCC
CAAGTAGCTAGGACTACAGACACATGCCATCATGCCAGGCTCATTTTAAAATTTTTTGTAGAGACGGGTCTCA
CTATGTTGCTCAGGCTGGTATTGAACTCCTGGGCTCAAGCAATCCTCCTGCCTCAGCATCTCAAAGTGTTGGG
ATTACAGGTGTGAACCATAGTATCCAACCTATTATGTTTTTTTAAAAAGAGATTGTTCTAATTATGTATATAA
TTAAAGTACATTAGGTTTAGTGATTGCAATTTGAGAAACCAATGTCCTTTCTTATTGAAAAAGGCCCTGGTAC
AGAAAGTGTTCTATCTTCCTCACAAGATCTGAGGTCAAAGGAGAGATTTTGAAACATTATAGCAATGGCTTCT
AGTTATTGAATAAAGAATGTCATTTGCTCCTCACAGGACCCCTTTCAGGGAGATTTCACATATTGGGAAACAG
CCTCATGAGCTTTCTACAAACATCTTTGTCCTTAAGTCTTCCCATTTCAGTAATTGGCTGTACCACTCATCCA
GCAGCTTTCAAAGTAAGAAACCTGGCAATCTTCCTTTAACATCCATTTCCCTTTCCCTCATTCCCCCATATCC
AATCATGTGTATTGGAGGTTTTTCCACCTCTAAACTTGAGAACCCATTTACTTTTTCTCCATCTCCTTTGCCA
TCACACTAGTCCAAGTGAAGTGTTTCATCATCTCATTTGCAGCATCCTTTAGTTTACCCTAGATTATCAATTT
TTGTCCCCCTCCCAATTCAGTCTCTACTCATGAGCTAACATGAGTTTATTAAGTGTTCATTCTGACCATGCCA
TTCCTTGCTCAAAATTCTCTAGTGCCTTCCCAATGCCCTTAAAATAAAATTCCTCATATGGCCTTTGTGGATC
TGCACTCCTACCTCTTCTCCTATTGCAACCAACCCTGTTACCTTTTTCTCTTTTCACACTGGTCTTCATTCTG
TGCTGCAAACTGGCTGAGATTCTTCCATCCTCAGGGCCTTTGCACAAGTTCTTCCTTTGCCTTAGAATTCTTT
CCTGGCCTGCACATGTCTTCATCCCCTTTCCCTAAGCTAATGCCTTACCCTTTAGGTCTCATTTTAAACATCT
CTTACCCAGGAAAAATTTCCCTGTCCAAACAAGTCTAGATTGGCTCCCCATTGTTTGTTCTAACTGTTCTATA
TTTCTCCTTCTTAGCATTTATCACAATTCTTGGTATGTTGTGCTTTTCACTTTCTTAGTCGCTATTATTGGAG
TAGGTAATACATCCATATAGTACAAAATTCAAAATGTATTAAAGGTATAGAGTAAAAAGTCTTCTCTTCCCTG
TCCCCCCGCTACCCAGTTCTCCTCTTTAGAACCACTTTTAGCATTTTGAATATCCTTCCAGAAATATTTTCTA
AGTATACAAGCAATTATGTATATAATATTCCTCTTTATTTTTGCCTTTTTATTAATACAGATACTATATGCAT
TTTCTGCACTTTATCTGTTAATAATATATCTTGGCAATTATTCAATATTAATATGTAAATAGTTGCCTCTTTC
TTTTTCATGCGCCATATAATATGGACTTATAATTTATTTAACCTCTATCTAGGTTGGTTTTAGGCATTGGTTA
TTACAAATAATGCTGCATTGAATACCTTTGTATGTAAAGTCATTTCCCACATCTATGCCTATAGAATAAATTA
CTAGAATTGAAATAATTCAGTTAAAGATTATGTGCATTTTTAATTTTGGTAGTTATTGCCAAACTGCTCTCAA
TGGAAGTTTTATGAATTCCCACTAGTGAATGCATTTGTCCTCACAATTATGTCAGTGAAATTTTTGTTCTTTG
CCAATGCAAGAAATGGAAAGTATCTCTTGGAAACTAATTTGCATGTTTTTTTTCCTTGCACAGTTATATTTTC
ATATGTTAAAGAGCCATTTGTATTTCTTGGTGAATTGTTCATAAACTTTGCTTACTTATTATCTAATGTCTGA
CTAATTTGCTACACTCTAAATTCCCAATTCATACTTAAGTCTCGCCCTTGCAATTTAGATGTACAATCTGCCT
CTTCAGTGGTTTCCACACCAAAAAATCCACAGGCTCCTGAATTTTATATGGGCCTAAGAGTCTTACTGAAAAT
TGGAAGTCTTAAGGTGTAGTCTATTTTATGCAGTGTGAGGCCTTTCATGCTCTGAAATGTACCACTCTTGACT
GACTTTTTCTTCTTCACCCTGTCCGGCACTGTGCCCTTATTAGCCAGAGAAATGTTCCCCCTTTCTCCCAGGA
AACGTTCTGACCTTACCCTGGTCCCTCAAGGACTCTCCTTCTCCCCCAAATTAACTTTCAAGGAGTTTCTCCA
ACCCAAAATGGCACCCTTCCTGAAAAGATTTCTAGTGGATACTGTTTTCTGTACCTCACTACGGATGTATTTT
GCTGCTTGGCTCCCCGTCTCCATTTTGCTATGGCAAACATTTTTTTCCAGAAATATATCAGATAAAATGAA
(SEQ ID NO: 119)
MDSDEGYNYEFDEDEECSEEDSGAEEEEDEDDDEPDDDTLDLGEVELVEPGLGVGGERDGLLCGETGGGGGSA
LGPGGGGGGGGGGGGGGPGHEQEEDYRYEVLTAEQILQHMVECIREVNEVIQNPATITRILLSHFNWDKEKLM
ERYFDGNLEKLFAECHVINPSKKSRTRQMNTRSSAQDMPCQICYLNYPNSYFTGLECGHKFCMQCWSEYLTTK
IMEEGMGQTISCPAHGCDILVDDNTVMRLITDSKVKLKYQHLITNSFVECNRLLKWCPAPDCHHVVKVQYPDA
KPVRCKCGRQFCFNCGENWHDPVKCKWLKKWIKKCDDDSETSNWIAANTKECPKCHVTIEKDGGCNHMVCRNQ
NCKAEFCWVCLGPWEPHGSAWYNCNRYNEDDAKAARDAQERSRAALQRYLFYCNRYMNHMQSLRFEHKLYAQV
KQKMEEMQQHNMSWIEVQFLKKAVDVLCQCRATLMYTYVFAFYLKKNNQSIIFENNQADLENATEVLSGYLER
DISQDSLQDIKQKVQDKYRYCESRRRVLLQHVHEGYEKDLWEYIED

An amino acid sequence for the protein encoded by the ARIH2 gene that is a regulator of T cell exhaustion/dysfunction resistance is available from the UniPROT database at accession no. 095376. cDNA NM_001349210, with gene location at Chr 3: 48918821-48986382.

(SEQ ID NO: 120)
ACTTCCGGAGCTGTGGGGACGACTCTTCTGGAGGAAGCAGCGCGGGCTTGACCGGCGTCGGCCCGCCGCCTCC
GCTGCCGCTTCGCCCCAATCCGGTCCCTCTGGCCCGGCCTGACCCGGTCTGGCTTGTTCGGGCTCAGCGGCCG
CGAGGCCGCAGCTCCCGATGGAAATCATATTATGTAGAATACTTGGGTGACATCTGCCTGAGAGATCTCCAAG
AATTACAGATTGAGTCTCGCTCTGTTGCCCAGGCTGGAGTGCAGTGGTGCAATCTCTGCTCACTGCAACCTCC
GTCTCCCGAGTTCAAGCAGTTCTCCTGCCTCAGCCTCCTGAGTAGCTGGGATTACAGAAGACAAAAATACTAA
TGCATTTGAGAAAGCGGTAGTTTTGGGGGGAGGGGGAAAAAGCAACTGCTTTCCTGATCTGCAACTTGGCTGG
ATGCTAAGATGTCAGTGGACATGAATAGCCAGGGGTCTGACAGCAATGAAGAGGACTATGACCCAAATTGTGA
GGAAGAGGAAGAAGAAGAAGAAGACGACCCTGGGGACATAGAGGACTATTACGTGGGAGTAGCCAGCGATGTG
GAGCAGCAGGGGGCTGATGCCTTTGATCCCGAGGAGTACCAGTTCACTTGCTTGACCTACAAGGAATCTGAGG
GTGCCCTCAATGAGCACATGACCAGCTTAGCTTCTGTCCTAAAGGTATCTCATTCAGTTGCTAAACTTATATT
AGTTAATTTCCACTGGCAAGTTTCAGAGATATTGGACAGATACAAGTCCAATTCTGCTCAACTGCTTGTTGAG
GCTCGAGTTCAGCCTAATCCATCAAAACATGTTCCCACATCCCATCCCCCTCACCACTGTGCAGTGTGTATGC
AGTTTGTGCGAAAGGAAAACCTACTCTCTCTGGCCTGTCAGCACCAGTTTTGCCGCAGCTGCTGGGAGCAGCA
CTGCTCAGTTCTCGTCAAGGACGGCGTGGGCGTGGGAGTCTCTTGCATGGCTCAGGACTGTCCACTCCGTACA
CCAGAGGACTTTGTGTTTCCATTGCTTCCCAATGAAGAATTGAGAGAGAAATACAGGCGCTACCTCTTCAGGG
ACTATGTGGAGAGTCATTACCAGCTCCAGCTGTGCCCTGGTGCAGACTGCCCCATGGTTATTCGGGTACAGGA
GCCTAGAGCTCGCCGAGTACAGTGCAATCGGTGCAACGAGGTCTTCTGTTTCAAGTGTCGTCAGATGTATCAC
GCACCCACAGACTGTGCCACAATCCGGAAATGGCTCACGAAGTGTGCAGACGACTCTGAAACAGCCAACTACA
TTAGTGCTCACACTAAAGACTGTCCCAAGTGCAACATCTGCATTGAGAAGAATGGAGGCTGCAATCACATGCA
ATGCTCCAAATGTAAACACGACTTCTGCTGGATGTGTCTAGGAGATTGGAAGACTCATGGCAGTGAATACTAT
GAGTGCAGTCGTTACAAGGAGAATCCTGACATCGTGAACCAGAGCCAACAAGCCCAGGCGAGGGAAGCCCTCA
AGAAGTACTTATTCTACTTTGAGAGGTGGGAAAACCACAATAAAAGCTTGCAGCTAGAGGCACAGACATACCA
GCGGATTCACGAGAAGATTCAGGAGAGGGTCATGAACAATCTGGGGACATGGATCGACTGGCAGTACCTACAG
AATGCTGCCAAGCTCTTGGCCAAGTGTCGATACACCCTGCAATACACCTACCCATATGCATATTACATGGAGT
CCGGACCCAGGAAGAAGCTGTTTGAATACCAGCAGGCTCAGCTGGAGGCTGAGATCGAAAACCTCTCATGGAA
AGTGGAGCGTGCAGACAGCTATGACAGAGGGGACTTGGAGAACCAGATGCATATAGCGGAGCAGCGGAGGAGA
ACCCTGCTGAAAGATTTCCATGACACCTAAGTTGGGATGTGGATGTGCCGGGGTGAGGAAGATGTGGCTGCAA
GGTCTCCCGGCTGCCATACTGCATGCTGCAGGCTCTGCCTTTCATGACCCCAGGCAACAGCCAGGGCCCCACT
CCTGAGAGACACTGGCAACACCTCTTAGTTGATTTCTGTTTTCTTCTCTTTTCACTTTTTGTTTCTACCAGGG
TAGAGGCCATGTTGAACTGGCCTCTTTTCAGGACTTTTATTTCCCCCTGGATGGTTGTTGGGAGGGAGGGAAA
GTGTTTTCTGAATGGCTATTAATAGTATTAGATCATTACAACTTATGTAACTTTCAAAGGTTGTACAATTATA
CAAAAAAAAAAAAAA
(SEQ ID NO: 121)
MSVDMNSQGSDSNEEDYDPNCEEEEEEEEDDPGDIEDYYVGVASDVEQQGADAFDPEEYQFTCLTYKESEGAL
NEHMTSLASVLKVSHSVAKLILVNFHWQVSEILDRYKSNSAQLLVEARVQPNPSKHVPTSHPPHHCAVCMQFV
RKENLLSLACQHQFCRSCWEQHCSVLVKDGVGVGVSCMAQDCPLRTPEDFVFPLLPNEELREKYRRYLFRDYV
ESHYQLQLCPGADCPMVIRVQEPRARRVQCNRCNEVFCFKCRQMYHAPTDCATIRKWLTKCADDSETANYISA
HTKDCPKCNICIEKNGGCNHMQCSKCKHDFCWMCLGDWKTHGSEYYECSRYKENPDIVNQSQQAQAREALKKY
LFYFERWENHNKSLQLEAQTYQRIHEKIQERVMNNLGTWIDWQYLQNAAKLLAKCRYTLQYTYPYAYYMESGP
RKKLFEYQQAQLEAEIENLSWKVERADSYDRGDLENQMHIAEQRRRTLLKDFHDT

An amino acid sequence for the protein encoded by the ATF7IP gene that is a regulator of T cell exhaustion/dysfunction resistance is available from the UniPROT database at accession no. Q6VMQ6, cDNA NM_001286514, with gene location at Chr 12: 14365676-14502931.

(SEQ ID NO: 122)
GCGCGCGAAAAGCTGAGGCGGCAACGTCGGGGACGGCTGCGCGGGACGGCTCTGTAGGAAGGAACTTGGTTCC
CCCTCCCTCAGCTTCCGCCCCAAAAGATTCAGAATGGACAGTTTAGAAGAACCTCAGAAAAAAGTCTTTAAGG
CTCGAAAAACGATGAGAGTGAGTGATCGTCAGCAACTTGAAGCAGTGTACAAGGTCAAAGAAGAACTGTTGAA
AACTGATGTCAAGCTGTTAAATGGCAACCATGAAAATGGAGATTTGGACCCAACCTCACCTTTGGAAAACATG
GATTACATTAAAGACAAGGAAGAGGTGAATGGCATTGAAGAGATTTGTTTTGATCCTGAAGGAAGTAAAGCAG
AATGGAAGGAAACACCCTGTATCCTAAGTGTTAATGTAAAAAACAAGCAGGATGATGATTTAAATTGTGAACC
TTTGTCTCCCCATAATATAACTCCAGAACCAGTCTCTAAACTGCCTGCTGAACCAGTTTCTGGTGATCCAGCC
CCTGGTGATCTGGATGCCGGAGATCCAGCCTCCGGAGTACTGGCCTCTGGTGATTCCACCTCTGGTGATCCCA
CCTCTAGCGAGCCCTCCTCTAGTGATGCTGCCTCTGGTGATGCAACCTCTGGTGATGCCCCTTCTGGTGATGT
GTCCCCTGGTGATGCCACCTCTGGTGATGCCACTGCTGATGATCTCTCCTCTGGTGATCCCACCTCTAGTGAT
CCCATCCCAGGTGAACCGGTCCCTGTTGAACCCATTTCTGGTGATTGTGCCGCTGATGATATAGCCTCTAGTG
AAATAACTTCTGTTGATCTGGCTTCTGGAGCACCAGCTTCCACTGATCCAGCCTCTGATGATCTGGCCTCTGG
TGATCTATCCTCTAGTGAACTGGCCTCTGATGATCTGGCCACTGGTGAACTGGCCTCTGATGAGCTGACTTCT
GAATCAACCTTTGATCGTACCTTTGAACCAAAGTCTGTACCAGTTTGTGAACCAGTTCCTGAAATTGACAATA
TAGAACCAAGTAGCAATAAAGATGATGATTTTCTTGAAAAAAATGGAGCTGATGAAAAATTAGAGCAAATTCA
GAGTAAAGACTCATTGGATGAGAAAAATAAAGCTGATAATAATATTGATGCTAATGAAGAAACTCTAGAAACA
GATGATACAACTATTTGTTCAGATCGACCTCCTGAAAATGAAAAGAAGGTAGAGGAAGATATTATCACAGAGC
TTGCTCTTGGAGAAGATGCTATATCTAGCAGTATGGAAATTGACCAAGGTGAAAAGAATGAAGATGAAACTTC
TGCAGATCTTGTAGAAACGATTAATGAAAATGTTATTGAAGATAACAAAAGTGAGAATATCTTAGAAAATACA
GACTCTATGGAGACAGATGAAATCATTCCTATTTTGGAAAAGCTTGCACCTTCTGAGGATGAACTTACTTGCT
TTTCTAAAACATCTCTCCTTCCAATCGATGAGACAAATCCAGATTTGGAAGAGAAAATGGAAAGTTCTTTTGG
TTCACCATCTAAACAAGAAAGTAGTGAGAGTTTGCCAAAAGAAGCCTTTCTGGTCCTCTCTGATGAAGAGGAT
ATTTCGGGTGAAAAAGATGAGTCTGAAGTTATATCGCAAAATGAAACGTGCTCTCCAGAAGTAGAAAGTAATG
AAAAGGACAACAAACCTGAGGAAGAAGAGCAAGTAATACATGAAGATGATGAAAGACCTTCTGAGAAAAATGA
ATTTTCTAGACGAAAACGTTCTAAATCAGAAGACATGGACAATGTACAGTCTAAACGTCGTCGATATATGGAA
GAAGAATATGAGGCAGAATTTCAAGTAAAGATTACAGCCAAAGGAGACATTAACCAGAAACTTCAAAAGGTTA
TACAGTGGTTGCTGGAAGAAAAATTGTGTGCGCTGCAGTGTGCTGTATTTGATAAGACTTTGGCAGAATTGAA
AACACGAGTGGAAAAGATTGAATGTAACAAGAGGCATAAAACAGTTCTCACTGAACTACAGGCCAAGATAGCC
AGGTTAACCAAACGCTTTGAAGCAGCCAAAGAAGATCTTAAGAAAAGACATGAACATCCACCCAACCCACCAG
TATCACCAGGAAAAACTGTAAATGATGTCAACAGCAATAATAACATGTCTTACAGAAATGCAGGCACAGTGAG
ACAGATGCTGGAGTCCAAAAGAAATGTAAGCGAGAGTGCACCACCATCCTTTCAAACTCCTGTGAATACAGTA
TCTTCAACCAATCTTGTCACTCCTCCAGCAGTTGTCAGTAGTCAACCTAAATTGCAGACTCCAGTGACTTCGG
GTTCCCTCACAGCAACGTCAGTTCTTCCTGCACCCAATACAGCTACTGTAGTTGCTACTACTCAGGTGCCTAG
TGGAAATCCCCAGCCTACAATCTCTTTACAGCCTTTGCCAGTGATTTTGCATGTACCTGTTGCAGTATCCTCC
CAGCCTCAGCTTCTACAGAGCCATCCAGGGACTTTGGTGACTAATCAACCATCTGGCAATGTTGAATTCATTT
CTGTGCAAAGCCCACCTACAGTGAGTGGTCTTACCAAAAATCCAGTATCCTTGCCATCCTTGCCAAATCCCAC
TAAACCAAACAACGTTCCTTCTGTGCCCAGTCCTAGTATTCAAAGGAACCCTACTGCCAGTGCTGCACCATTG
GGAACAACACTTGCTGTGCAGGCTGTTCCAACAGCACACTCTATTGTACAAGCCACAAGGACTTCTTTACCCA
CAGTGGGCCCATCAGGACTCTATAGTCCATCAACTAATCGAGGTCCTATACAGATGAAAATTCCAATTTCTGC
ATTTAGTACTTCGTCTGCTGCAGAACAGAACAGCAATACCACCCCAAGAATTGAAAACCAGACAAACAAAACA
ATAGATGCTTCTGTCAGTAAGAAAGCAGCTGATAGCACATCACAGTGTGGAAAAGCCACTGGCAGTGATTCAA
GTGGTGTCATTGATCTCACAATGGATGATGAAGAGAGTGGAGCTTCACAAGACCCCAAAAAACTAAATCACAC
TCCTGTATCAACCATGAGTTCTTCTCAGCCTGTGTCACGACCATTGCAACCCATACAACCAGCACCGCCTCTT
CAACCATCTGGGGTGCCAACAAGTGGACCATCTCAGACCACCATACACTTACTACCTACAGCTCCAACTACCG
TGAATGTAACACATCGTCCAGTAACTCAGGTGACCACAAGACTCCCTGTACCAAGAGCTCCTGCAAACCACCA
GGTGGTTTATACAACTCTTCCTGCACCACCAGCTCAGGCTCCCTTGCGAGGAACTGTTATGCAGGCTCCTGCT
GTTCGGCAGGTCAATCCCCAAAATAGTGTTACAGTTCGAGTGCCTCAAACAACCACATATGTTGTAAACAATG
GACTAACCCTGGGATCAACAGGACCTCAGCTCACAGTGCATCACCGACCACCACAAGTGCATACTGAGCCCCC
ACGCCCCGTGCACCCAGCACCCTTACCAGAAGCTCCACAACCACAGCGTCTGCCCCCAGAAGCTGCCAGCACA
TCTCTGCCTCAGAAGCCACACTTGAAGTTAGCACGCGTTCAGAGTCAAAATGGCATAGTACTGTCATGGAGTG
TCCTGGAGGTGGATCGAAGCTGTGCCACTGTTGATAGCTACCATCTCTATGCTTACCATGAGGAACCCAGTGC
CACTGTGCCCTCACAATGGAAAAAGATTGGGGAAGTCAAGGCACTTCCCTTGCCCATGGCATGTACTCTCACC
CAGTTTGTATCTGGTAGCAAATACTACTTTGCAGTACGAGCCAAGGATATTTATGGACGTTTTGGGCCTTTCT
GTGATCCTCAGTCAACAGATGTGATCTCTTCTACCCAGAGCAGTTAAACCTTGGAGCCTTTATATTTTCCTCT
TTTAAAATTTCCACCTTTTGGTCTTGTTTTTAATCTTGTGCATGATACCCCATGTAAAATCCACCTTGTGCAA
GATTTCTTGGACAGATGTGTGTATACACTACATTTGTTTATAACCAGAAGCAAAATAAACTCAGCCCACAAAG
CTAGAATCTTTTCCTGGACAGTTTAGGCTTTGGGGTTTGGAAATGTAAATGTGTACCTTGCTTTAGTTTTGAG
GCTGGGGAATATGTGTGGGTGTTTATGTGTGTTTTTCCTTATGTAGGTGTTATTGCATTGGAGTCTCCCATTT
TCATTCTCAAATTTACCTCTTAAAGTACGAAGTAAGTAGATCAAAGGATTTGAGATGTGTAACTGGCATGATT
CTGCTTTTGAAGGATCTATAGTATCATTTTAGTTAAGTGGGTCAAACAGAATCAAAACAAAACCCAAAGAAAT
AAATAAAAAACAAAATGGCTAAATAGTTTAAAATAGGTTAATTCGAACACAGGAAAGGATCTATTTGTTGTTT
CTTTTGTCTGGTCTCCTGAGTTGTTAATTAGGTGAAAAAAGATCTGCAATGGCCCCCTCCCTTTCCTAATCTG
GCTTTTACATTTATTTTGTGCCTTAAAGATTAACTACAAAGATAAACATGGCCAAAAATAAATAAATAAATAT
GGCCATATGTCCGTTGTTGCTTAGTCTTCCCTTGCAGCCTTTTACCCTTGATTTCTCCTTCATCTCTACCAAA
TATAGCACAACTCCTCAAGTAATTTTTTTTTTTTAAGATGGAGTTTTGCTCTTGTTGCCCAGGCTAGAGTGCG
GTGGCACAATCTTGGCTCACTGCAACCTCTGCCTCCTGGGTTCAAGCAATTCTTCTGTCTCAGCCCCAGAGTA
GCTGGGATTACAGGCACCTGTCACCATGCCCGGCTAATTTTTGTATTTTTAGTAGAGATGGGGTTTCACCATG
TTGGCCAGGCTGGTCTTGAACTCCTGACCTCAGGTGATCCACCCGCCTCGGCCTCCCAGAGTGCTGCGATTAC
AGTTGTGAGCCACTGTATCCAGCCTACTCAAGTGATTTTTAAACCAAGGTGTGTGTATGTACATGGATGTGTA
TGTACACACACACACAGGTGCATGCATAGTCTCATCTTAGCCGGGCATGGTGGCGACAATCACATTTAACTCC
AAAAATTTGACGTGCTATTTTCTTCTAAAACTATAGATGCTTTTATTTTTGGTCACTATTTAACTTTGTGTAA
AGTGGACCAAGAGAAAACCATATTGTGGAAGACAGTTTTTCACACATTTTAGTATCCGTGTTGCACACTGTGT
TAGAGATTATGAAAACCATTCTAGTTCAGTTTATCACCCAAAGCTATCACCCAACACTAACTCCTTAGTATTC
TTTCAAAGGAAGCAAATAGTAAGATTACAATATAGAATGAGCTAAAATATCAAGGGGAAAGCTTCATTTCCTC
TTTATTCTCTTTATCGTCCTTCCCATTTTAACCTTTTAACTTTGTTTTGATGGTGCCAAGTTGAGTCTGATGT
ACCCATTGTTAGCTTTGGATAGAATTTAAAGTATATCCCAGAATTAGCTGGGCATGTTGGTGCATGCCTGTAA
TCCCAGCTACTCAGGAGGCAAGGCTGGAGAATCGCTTGAACCCGGGAGGCGGAGGTTGCAGTGAGCTGAGATT
GCGCCATTGCACTCCATCCTGGGAGACAAGAGCGAAACTCTGCCTCAAAAAAAAAAGAATTTAAAGTATATCC
TTTGAGGTCATTTTATGTAACAATTAGATATAACCTCCTGTGTATCTCTCTGTTCCTCTTCAAAGTTATTAAA
ATTCAGCTTAGGTCATGGATTTTTAATATGAGGGCCAGCTGTACTGGTGTCAGGTAGGTGTTTTGGGTGTAGA
TAATTTAGGACTGGAGGGCTGAGTTAAGTTTTAGGAGGAGAATGTGGTTGAGAATATTTTGAAAGGAATTTAT
GGAGTTTGTGAAATAACTTCGAAGTCCTCTTCCTTTACAATATTTGAATTCATATTTGTACCTTCTCAAAATA
GTGATTCATTTTTCCTAGAATTACAGGAGGGAGCTCTTTTACTAATGTTGTTTTGTTTGCAACTTTGATGGCT
TATAATAGGAAGTATTCTAGTTGTAAAGAAAACTCTTTAGAGACTTTTGACTGGTCAGTATACTGAGGTGTGA
GATTTGATTCATGATGAAGAAAGCCTATAGATTGCCAAAAAATTAATTCTCCAAACCACCTTTCACTCTCAGA
AAATGAGACCACAAAGGAGTATGCTATAAATCAAATTTGCCAACCAATTATGTAGATATTACTCATTCTAGGA
CTAATGATGATGGTAAAGAAGTTGCCAGTGTTATGGCAATGAAAATTTCAGAAAGGAGGAGTTGATGATCTTC
TAGATGTATATGAACACCTGTCTATATCTGCATGTATATGTTTTGACCTGCAGTGGTTGCAATGTTGATATGT
GTTCAAGATTATTCCTGTCTACAAAACTGAAGGCCCATGTTCAAATTGTTCTTTATTGGGTGTTTTTATGGTC
ACGTGGTAACAATTTTCTTACCTAACCTACAAAAGGTTCTCTTGATGAACATTTTTATTTATATTTACTAATC
TTTTTTAAAAAAAGCTTTCATAGCATTATATAATCAGATGAAGAAAGCCCAGTAGAATAAAAAAAAAATTCAT
TAGCCTAGCCTATATTATGTTTTCTGTCAAAGGAAAACAAATTCTCAAATAGGAATTCTAAAAATATTTACTA
AAGTAAAATAACTACTTAAAATGTTTTATTCCAGTTGGAAGGAAGGTACAGGGAGAAATCGCAATTATTTAGG
GGAGAAGTATATTTATTATAAGATGGTGTCCTCAAATTAGCCTACCATGGCACGTAGGGGCAGCAGCTATATT
AGATTTACTAGAGGTGCTAAGTTAGAACACTAGGCTTTTATTGAGGCAGGTTTTAATATTGATAGATGCTTTT
TGTTTGGTTTGTTTCTTCTGGGAGAGAATGGAGGACTTAAGTAGAAGTAGCTACTGATAACAGACTTTCTAGT
AGCAGTTTCCACTCCACGGTTACCTTTTTAGTTTCATAGTATCTTTTCACAAAGTATTACAAATAAGCTAGAT
TCTCCCAGTTTGGGAATGCAAGTTTGCTACATTTTTAGCCTGGCAATATTTGTGTAGGTATTGCCTTATTGGA
AATTCTGGAAACCTGATACTGCAACCTGCAATGTAGGATGTTTGTATGGCATTTAAAGGTAATGGTGATGTTT
ATTATTCTATACTTTGCATTCTGTGAGAGTAATTTTCACTCTGTCTTAAGTGTGAGTAAGCCTCTTCTAAAAA
TCTTGTTCTTGCCAAGAAATTTATAAATCACATACGAAGACGTCTGTTGCTAACAGTTAACTTTATGAGGTAA
CTATATCCTTCTATTTCTCTGGACTCATTTTTAAAAAATATGCCGAATACTGCATACTGTTTAAGGTAGTATA
TAAGTTTATGAGAGAAGTGGAGAGCTTTCTTCCTTGAAAAGTCGGTATTTGTTGAGATACCATTTGCCTCACA
GAGAGGTGTTCCCCACTCCCATCCCCATTGCCAGATAATAAATATTTTGAGAAAAGTGACCTAAAACAGCTGA
AATCTTAGGTGCATCTGTCTGCAGACCTCCTTAAGCAGGCTGTATCTTACAATTCCCTTACTGCACTGGGTAA
GTGTTAACTTAGTTTTTGTTGTTTGCTCTTTTGCTTTAAATATTCTCCAAATTACCATTTATGCAACATGGTT
AGGGTTAATACTGCATGGTATTCATTTATCTTGTTTCATGAACTTTCCAGTACTGTACAAGGTCAACAAAGTA
ATGCCTGTGGTATCCTCATCTCTCACTTTTTTACTCTGTGATTTTAGCACAGTAAGGTACTGCAAAGACCTTC
CTTCCAAATGTTCTCCTTGACTTTATTTCTTGGGCCAATTCAGTATCCTCAACATCCTAAGATTTTGTTGTTT
TATCACTGACCTGTGGTTGGCCTGTTTTATTCTAATTTCCAGAAAAGTCAAGTCCCAGTATTTGCAATATCAA
ATAACTCTAAAACCGATGTGTGATTCTACCTTCCTTACTATTTTTACTGGGCAAATGCCCTATTTTTTTAATT
ATTATTATTTTTAACTTTTGGGACACACAAAAATCAGCAATTCTCATGAAGCGTTTGTTAGTGTGGCAGACTT
GTCTAATTCCTGAAACTCATTCATCCCCTTGAGCCAGCCAATGGGGAGGAATAGGATAATGCAAACACATGTT
TTGTTTTCTCATTTTCAAATAATTTACCATGTTAAAATAAACTTTTCTTTGTTTTTTATTTGTAGAGTCAGCT
AAGTACCCATATTTAAATGCCGTCTTTATTATTTTTTTGAGGTCTTTGTTTTTGTCTGTTTTTGTTTTGTTTT
GTTTTGTAAATAAGGTAACTGGGCAATCAAACACCTTTTGGGGATTCTGGCTTTAGTATTTTATCAGCCATTT
TAAAATTAAATATAAAAATCCTTTGTAAGAAACTTGCATCCTAATTTTTCTTTATTGCAATTGAAAGTGTAAA
TAATAAGACAATGTAAGTAAGACCTTCCTAATGTCTAATACAAACTGGGCTCCAGCAAGTGGCCCTATTTTTA
TTAGGGTTTTGAAGGTTTGTGTGTGTGTGTGCGTGCGTGTGTGTGTTTTTCTTTTTTAAATGTATAGTAGAGT
GGTGTCTGTATAAGTGTTACCTGTAGTGGGGTTTTGTCCAGCAAGCCTGAAATTTATACTTTGAAATAAAACT
ACTGGGTTTTTAAC
(SEQ ID NO: 123)
MDSLEEPQKKVFKARKTMRVSDRQQLEAVYKVKEELLKTDVKLLNGNHENGDLDPTSPLENMDYIKDKEEVNG
IEEICFDPEGSKAEWKETPCILSVNVKNKQDDDLNCEPLSPHNITPEPVSKLPAEPVSGDPAPGDLDAGDPAS
GVLASGDSTSGDPTSSEPSSSDAASGDATSGDAPSGDVSPGDATSGDATADDLSSGDPTSSDPIPGEPVPVEP
ISGDCAADDIASSEITSVDLASGAPASTDPASDDLASGDLSSSELASDDLATGELASDELTSESTFDRTFEPK
SVPVCEPVPEIDNIEPSSNKDDDFLEKNGADEKLEQIQSKDSLDEKNKADNNIDANEETLETDDTTICSDRPP
ENEKKVEEDIITELALGEDAISSSMEIDQGEKNEDETSADLVETINENVIEDNKSENILENTDSMETDEIIPI
LEKLAPSEDELTCFSKTSLLPIDETNPDLEEKMESSFGSPSKQESSESLPKEAFLVLSDEEDISGEKDESEVI
SQNETCSPEVESNEKDNKPEEEEQVIHEDDERPSEKNEFSRRKRSKSEDMDNVQSKRRRYMEEEYEAEFQVKI
TAKGDINQKLQKVIQWLLEEKLCALQCAVFDKTLAELKTRVEKIECNKRHKTVLTELQAKIARLTKRFEAAKE
DLKKRHEHPPNPPVSPGKTVNDVNSNNNMSYRNAGTVRQMLESKRNVSESAPPSFQTPVNTVSSTNLVTPPAV
VSSQPKLQTPVTSGSLTATSVLPAPNTATVVATTQVPSGNPQPTISLQPLPVILHVPVAVSSQPQLLQSHPGT
LVTNQPSGNVEFISVQSPPTVSGLTKNPVSLPSLPNPTKPNNVPSVPSPSIQRNPTASAAPLGTTLAVQAVPT
AHSIVQATRTSLPTVGPSGLYSPSTNRGPIQMKIPISAFSTSSAAEQNSNTTPRIENQTNKTIDASVSKKAAD
STSQCGKATGSDSSGVIDLTMDDEESGASQDPKKLNHTPVSTMSSSQPVSRPLQPIQPAPPLQPSGVPTSGPS
QTTIHLLPTAPTTVNVTHRPVTQVTTRLPVPRAPANHQVVYTTLPAPPAQAPLRGTVMQAPAVRQVNPQNSVT
VRVPQTTTYVVNNGLTLGSTGPQLTVHHRPPQVHTEPPRPVHPAPLPEAPQPQRLPPEAASTSLPQKPHLKLA
RVQSQNGIVLSWSVLEVDRSCATVDSYHLYAYHEEPSATVPSQWKKIGEVKALPLPMACTLTQFVSGSKYYFA
VRAKDIYGRFGPFCDPQSTDVISSTQSS

An amino acid sequence for the protein encoded by the ATF7IP2 gene that is a regulator of T cell exhaustion/dysfunction resistance is available from the UniPROT database at accession no. Q5U623, cDNA NM_001256160, with gene location at Chr 16: 10326434-10483638.

(SEQ ID NO: 124)
AGCATTGTTAACGGCTGCGGTTCGAAGTGGCTGTGGGTATTGCTGCGGCTGGTGCGTCGTCGCTCTAGGTTTT
CTTCCGCCGTCTGTGACAGCTTCTTGATCTGTCCCCAGGAATCCTGCTTTTTTTTTTTCTTTGAGACACGGTC
TCACTCTGTCACCCTTGCTGGAGTGCAGTGGTGCAATCACAGATCACGGCAGTGTCTAATTCCTGGGCTCAAG
TGAACCTTTTGCATCAGCCTCCTGAATAGCTGAGACCACAGGATATGAAAGATGGCAAGTCCAGATAGAAGTA
AACGGAAGATATTAAAAGCCAAAAAGACAATGCCCCTAAGTTGCCGGAAGCAAGTAGAGATGCTGAATAAGTC
AAGGAATGTTGAAGCGCTGAAAACAGCAATTGGGAGTAATGTTCCAAGCGGTAATCAGAGTTTCAGTCCTAGT
GTCATAACTAGGACGACTGAAATAACCAAATGTAGCCCTTCTGAAAATGGTGCATCCTCATTGGACTCTAATA
AAAATTCAATATCAGAGAAAAGTAAAGTATTCTCTCAGAATTGCATAAAACCAGTAGAAGAAATTGTTCATTC
AGAAACAAAATTGGAACAAGTTGTTTGTTCGTACCAAAAGCCAAGTAGAACAACAGAATCCCCCAGCAGAGTC
TTCACAGAAGAGGCAAAAGATTCACTGAACACTTCTGAAAACGATTCTGAGCATCAGACAAATGTAACAAGAT
CCCTTTTTGAGCATGAGGGGGCTTGTAGTCTAAAGTCCAGTTGCTGTCCACCCAGTGTATTGAGTGGTGTTGT
TCAGATGCCAGAGTCTACAGTAACCAGTACCGTGGGTGACAAGAAAACTGACCAGATGGTTTTCCATTTAGAA
ACAAACTCCAATTCAGAATCACATGATAAAAGGCAAAGTGACAACATTTTATGTTCAGAAGACTCAGGTTTTG
TGCCTGTTGAGAAAACACCTAATTTGGTGAATTCAGTCACTTCTAACAACTGTGCTGATGACATTTTGAAAAC
TGATGAGTGTAGTAGAACCAGTATTTCAAATTGTGAAAGTGCAGACTCAACATGGCAGTCATCACTTGACACT
AATAACAACAGCCATTATCAAAAGAAGAGGATGTTTTCAGAAAACGAGGAAAATGTTAAACGCATGAAAACTT
CAGAGCAAATTAATGAAAATATTTGTGTAAGTTTGGAAAGGCAAACAGCATTCCTGGAACAGGTCAGACATTT
GATTCAGCAGGAGATCTATAGCATAAATTATGAACTATTTGATAAGAAACTGAAAGAATTGAACCAACGCATT
GGGAAGACAGAGTGCAGAAATAAGCATGAAGGAATAGCTGATAAACTTTTGGCAAAAATAGCAAAACTTCAAA
GACGTATTAAAACAGTATTATTATTTCAAAGGAATTGTTTGAAACCAAACATGTTATCCAGTAATGGAGCCTC
TAAGGTTGCAAATTCAGAGGCTATGATTTTGGATAAGAATCTTGAGTCAGTTAATAGTCCAATTGAAAAGTCT
TCTGTGAATTATGAGCCTTCTAACCCTTCCGAAAAAGGAAGTAAAAAAATTAATTTGTCATCAGATCAAAATA
AGTCTGTTTCTGAAAGTAACAATGATGATGTTATGTTGATTTCTGTGGAAAGTCCTAATTTGACAACTCCAAT
TACATCAAATCCAACAGATACCAGAAAAATTACATCAGGAAATTCTAGCAATTCTCCCAATGCTGAAGTTATG
AAAGTCCAGTATCCCCCCTGGAGTCACATTCGAAAGCTGCTTCAAACTCAAAGGAAACAACCCCATTGGCACA
AAATGCAGTCCAGGTTCCTGAGTCCTTTGAGCACCTGCCACCTCTCCCAGAACCACCAGCACCACTACCTGAA
TTAGTAGACAAAACCCGAGACACACTTCCTCCCCAGAAGCCTGAGCTCAAAGTGAAACGGGTTTTCAGACCCA
ATGGCATTGCCCTGACTTGGAATATAACCAAAATCAATCCCAAGTGTGCTCCTGTAGAAAGCTACCACCTCTT
CCTGTGTCATGAGAACTCTAATAATAAGTTGATTTGGAAGAAGATTGGAGAAATTAAAGCTTTACCACTCCCC
ATGGCCTGTACTTTATCTCAGTTTTTAGCTTCCAACAGATACTATTTTACTGTCCAATCAAAAGATATTTTTG
GACGATATGGACCATTCTGTGATATAAAATCTATCCCTGGGTTTTCTGAAAATCTTACGTAAAAGGTGTTTAA
TAATGATATACTACTTTTTTTTTCATATTTGTTTGTTTGCAATGTTACTGTAATACTATTTGCCATTGTAAAA
GGCCAGCTCAGATTGTGAGCCCTTTCTATTGGGACAGTCCTCTTCTATATGTTTTAAGTGGCCAGTAATTTAA
TGAATTTCTGGTTTGTATTAAATATGTCCTTCCAATGGATAAGTTCTAAAACATACGCTATCATTGGCCCATG
TTGCTGAGGTGCATTGTGAACAATACCTTTAGTGACTGTGGAACTGCTGCTTCTATCAGAAGACCTAGGATAC
AAGCAGCCACTGTTTCCTCCGTTAACAATCAGCTTTTAGTAACCTGCTGTGGTCAGCATAGCTACAGGAAAAG
TCAGCCCTTGACTGAGGGCCAGATCATCCCTGGAGTGCACCTCTACTAAGATTTTATGAAAAGATGACATGTT
GGGCTGCATGATAAAAGTTAATTATAAAAATTAAAAGATTTTTTTTTTTGAGATGGAATATTGCTCTGTTGCC
CAGGCTGGAGTACAGTGGTCACTGCAACCTCCATCTCCCTGGTTCAAGCAATTCTCCTATGTCAGCCTCCCAA
GTAGCTGGGACTATAGTCACTTGCCACCATGCCCGGCTAATTTATTTTTAGTAGAGATGAGGTTTCACCTTGT
TGGTCAGGCTGGTCTCAAACTCCTGACCTCAGGGGATCCACCCGCCTCGGCCTCCCGAAGTGCTGGGATTATA
GGCGTAAGCCACCATGCCTGGCCAAAAATTAAAAGATTTTATGAAACGAAATGGCTTCCCATTCTTTCTATTC
AGCTTCTAAGTGGGCTATTCATTTGTGCTCTCCTCTTTTTTGTTTTGTTTTGCACTTTGTCAATCATTTGGGG
AAATAGCCTGGAGGTCTTTCCTGAATCTGTTTGTAGACATAATAAAATTTGTGTGCTGTGTACACAAAAACAT
AATGTATCTTATGAAAGCATTACCCAACCTGTTTGAGTTGAGAATGCATTTGTCCACCTTTGATTTGAGGCTT
TAGTTTGCTGATTTCCTAAATGCTACCCTTTGATCATTTCCTGGCCACCATCACAATACTAAGGGGCTCAGAT
GTGTCTTGTGCCCACCTCTTCCTGAGAAGAGATGGAAGTGGAGCTGTGACTAGTACAAGCAGCCCAAGAAACT
CTGAACGGGGCCCAATGGAGGCAAACTTGAGCAAATAATTGGGATGATAAGAAACAAAAATAATCCCAACTGT
TTGAAAGTTCGAAAGAGGGGCATTCTTTTCTGGTACATGGTGAGAGTTCAGGCTCCAGAGACTCAAAACGGAA
TGGTTTTCCTTGGCATTTTGTAAATGCTCTCACATCTGTAGCAATAAAGCTGTTATTTTCTGCA
(SEQ ID NO: 125)
MASPDRSKRKILKAKKTMPLSCRKQVEMLNKSRNVEALKTAIGSNVPSGNQSFSPSVITRTTEITKCSPSENG
ASSLDSNKNSISEKSKVFSQNCIKPVEEIVHSETKLEQVVCSYQKPSRTTESPSRVFTEEAKDSLNTSENDSE
HQTNVTRSLFEHEGACSLKSSCCPPSVLSGVVQMPESTVTSTVGDKKTDQMVFHLETNSNSESHDKRQSDNIL
CSEDSGFVPVEKTPNLVNSVTSNNCADDILKTDECSRTSISNCESADSTWQSSLDTNNNSHYQKKRMFSENEE
NVKRMKTSEQINENICVSLERQTAFLEQVRHLIQQEIYSINYELFDKKLKELNQRIGKTECRNKHEGIADKLL
AKIAKLQRRIKTVLLFQRNCLKPNMLSSNGASKVANSEAMILDKNLESVNSPIEKSSVNYEPSNPSEKGSKKI
NLSSDQNKSVSESNNDDVMLISVESPNLTTPITSNPTDTRKITSGNSSNSPNAEVMKVQYPPWSHIRKLLQTQ
RKQPHWHKMQSRFLSPLSTCHLSQNHQHHYLN

An amino acid sequence for the protein encoded by the ATXN7L3 gene that is a regulator of T cell exhaustion/dysfunction resistance is available from the UniPROT database at accession no. Q14CW9, cDNA NM 001382308, with gene location at Chr 17: 44191805-44200113.

(SEQ ID NO: 126)
ATGAAAATGGAGGAAATGTCTTTGTCTGGCCTGGATAACAGCAAACTAGAGGCCATCGCTCAGGAGATATACG
CGGACCTGGTCGAGGATTCTTGTTTGGGATTCTGCTTTGAGGTACACCGGGCTGTCAAGTGTGGCTACTTCTT
CTTGGACGACACGGACCCTGATAGCATGAAGGATTTTGAGATCGTGGACCAGCCGGGCTTGGACATCTTTGGA
CAGGTTTTCAACCAGTGGAAGAGCAAGGAGTGTGTTTGCCCCAATTGCAGTCGCAGCATTGCCGCCTCCCGCT
TTGCTCCCCATCTGGAGAAGTGCCTGGGAATGGGTCGGAACAGCAGCCGAATCGCCAACCGCCGGATTGCCAA
TAGCAACAATATGAATAAGTCTGAGAGTGACCAAGAAGATAATGATGACATCAATGACAACGACTGGTCCTAT
GGCTCGGAGAAGAAAGCCAAGAAGAGAAAGTCAGACAAGCTATGGTATCTCCCATTCCAGAACCCCAATTCCC
CTCGAAGATCCAAGTCATTAAAACACAAAAATGGGGAACTTAGCAATTCGGATCCTTTTAAGTATAACAATTC
AACTGGGATCAGCTATGAGACCCTGGGGCCGGAGGAGCTTCGCAGCCTGCTAACCACGCAATGTGGGGTGATT
TCTGAACACACCAAGAAGATGTGCACAAGGTCCCTGCGCTGCCCACAGCACACAGATGAGCAGAGGCGAACCG
TACGGATTTATTTTCTCGGGCCCTCGGCTGTCCTTCCAGAGGTCGAGAGCTCCCTGGATAATGACAGCTTTGA
CATGACTGACAGCCAGGCCCTGATCAGCCGGCTTCAGTGGGACGGCTCCTCTGACCTCTCACCCTCTGATTCA
GGCTCCTCCAAGACGAGTGAAAATCAGGGATGGGGTCTAGGTACCAACAGCTCTGAGTCACGGAAAACCAAGA
AAAAGAAATCCCATCTGAGCCTGGTAGGGACTGCCTCCGGCCTAGGTTCCAACAAGAAGAAGAAGCCAAAGCC
ACCGGCACCCCCGACGCCCAGCATCTATGATGACATCAACTGACTTGGGTGCAAGGGATAGCCTTTGGCTGAG
CAGAGCCCTCTCTCCTGACCCCCACCCAGGTGGCATAGCCTGGCAAATGGACGGCTGCTGGGGGTTGGGGCTT
GGGAAGCTTGGTGGGCCAGGCAGGCAGAGGATCCGATTATGCGCCCCTGGGGGGTGTCAAAGTCCTCTGCAAT
GGAGCACGACTCCTCGGCGTGCAGGACTCAGACCTGGACATATTATGCCTTGGACATAAGTTTGGTGGGGAGG
CGGTTTTCCTATTTATTCTGTGAGTTACTGGATGTCCAGCTAGGCCAGGGGCCTGACCTGGGCACTGTGCCAG
GGCACTGCCTGCCCCCCACCCCAGGAACTGGACTAAGCCCTGCTGAGGGGCAATGTGGCGACCCGGGAGGCTG
TGGGTTGGAAGCTGAGCCTGGAGGGGGCCTGCCTCGGCCGCCCTCAGCAATGTGAATGGGTCCGGTGCTTGGA
GCTGAGGGGCAAGGCTGGGCATGTCCTGTCTGTGTGCAGAATCTTATCAAATGCCCCCAGTCCCAGGGGTAGG
CAGGCACAGAGTGCTGTCTGCTGAGGTGGGCGTCCAGAACTGCCGCCACCTTCCCTGCCCCTCACAGGGGCAG
CCCTTTCCCCTCAGTCCCCATGGGCCTCCTTGGCAGCAGGAGCTGTCCTTTTGTTGGGTGCCAGGAAAGGGCC
TCCAGCCTCTACAGCTTCAAGGAATGGGAAAGGGAGGGTAGGAAGAGGGAGAAGTGTCAGAATTCCTCCCCCT
GCCTCTCCTCCCTGACCCAGGGCTGGTGAGGAGTGGGGCCCCAAGGTGAGAGGGAACGGTGCTGCTTTATTTG
AAATGTTTTCTTACCTCATTCCCTGCCCCAGGAAGGGACCCAGCCTCACCCTCCTGGGGTGGCCCCGTGTTCC
TCCTGCCCACCCTGCCCCACTGCCCTACCGGGGCAGCCCAGGTTCCCAACTGCTGCTTCCACCCCCTTCACCT
TGACTGGCTTCAGTTCCTCTCAGCGCTCCTGCCTCCAAAGCGTGCCCTGTTTCCCTTCCTTAGCGCTTTTAAG
TTATTTATTCTGTACTTGTGGTTTGGGGCTCTGAGGAAAATCCTAATCTTGTGCCTCTCCCCCTTTGCTAGGA
GTTGGGTGGGAAGAGAGGGTGGCCTCTGTGCCCCAGGTTGTCAGTGGAGGGGAAGGGGTGTCACTGCCCCTCT
GTGGGACTGTCATGCCAGTGTGCCCACCTGCCTGGTCTACATCCTAAAGAGATGTACCGTCCCCCCTCCATGC
GGGCACCACCACTCCCAGGAGCCAAGCTGGAGGACCGCGACCTGGGCTGGGAGCAGAGCTGACTGACATGGGT
GGAGGTGACCCTGAGCCAAGTTCTCTGGCACTTGCTGGACATGTCCCCTACCCTCTTCCCAAGAGGAGCCATC
CCACATTTGGGTAGTTAGTACCCAGGACTGGTTGGACTGATCGGGTTAGGGACCCTAGAGGGTTAAGGGAACA
ACAGAGATGGGGCTGCAATGCCCTGTCTGGAACTCCGCTCTCCCGCTGGGGTGGTTCTGATTGTGGCTCATGC
CTGGTTGCAAATTGGTTTTTAACCCACAAATTGTGATTATTTTTTAAAAAGCCAAACAGATTTTGGCAGGAGT
TAGAATAAATCCCGGGGCCTGGGCTTCTCTATTCTTGGCCCTCCGTAGTCTCCCTCCGAGAGGAGGCCAGAGG
GAGAGGAGGATCTCAGCAGGCCTCTAGCCGCTTCCAGATGCAGTCTGGGGAGTGGGCTGAAGGTGAAGGGAGG
AAGGCTGTGGGCCACTCTCCCTTCTGAGAGGCAGCTGCAGCTCAGGCCCTAATACACTCTTTGCCCTGTGGCC
TCTCCCTTTTCCCCCCTTCTGGTTGGAGGAGGGAGAAGTGGGAAGTAGCTTGGGAACTGGTTTGTCCACATAA
ACTTCCCCATTGTTCCTTGGCCCGCCCTCAGGGCAGAGCCCCCTGCCCAGGCTGGGTAAGAGATGGGCTTGGT
CCAGCAGGGACCCTGAGGGAACAAACCCTTTTCCTTCTGGGGAGAGAGTGCCCCCCCTACCATGTAGTTGAAC
AGGGGCTAGGAGCTCCCCACTCCCCTCCCTCTAACAGCAGGCTGTGTGGGTTTCAATTCCCATCCTTCCCACC
CCGGCTAGGTGTCGTCCACCCTGTATCCTGTGTCTGAGTGTGTGTGGGGGGGTTCTGTACTAATTTCCATGGC
CGGTGGCTTTTCCTTCCATGCATCACTCCCCCCCGCATGCCCAGGGGCCACCCGCCTGGCATTACCGCATGCT
GGGGTCATTGGGGGAGGGGGGTGGGGCTCACGCTGTCCTGTGGTCTTGAGATTTTTATTTTTGCATATGTAAT
CCATTCTGTACAGGTAGCTAACTTTGTAAACGCTGTGTATTCCCTCTGCCCCCATGGCTGCTGGTGTAAATAA
ACTGCATCTCCCGTTGGTA
(SEQ ID NO: 127)
MKMEEMSLSGLDNSKLEAIAQEIYADLVEDSCLGFCFEVHRAVKCGYFFLDDTDPDSMKDFEIVDQPGLDIFG
QVFNQWKSKECVCPNCSRSIAASRFAPHLEKCLGMGRNSSRIANRRIANSNNMNKSESDQEDNDDINDNDWSY
GSEKKAKKRKSDKLWYLPFQNPNSPRRSKSLKHKNGELSNSDPFKYNNSTGISYETLGPEELRSLLTTQCGVI
SEHTKKMCTRSLRCPQHTDEQRRTVRIYFLGPSAVLPEVESSLDNDSFDMTDSQALISRLQWDGSSDLSPSDS
GSSKTSENQGWGLGTNSSESRKTKKKKSHLSLVGTASGLGSNKKKKPKPPAPPTPSIYDDIN

An amino acid sequence for the protein encoded by the BORCS8 gene that is a regulator of T cell exhaustion/dysfunction resistance is available from the UniPROT database at accession no. Q96FH0, cDNA NM_001145783, with gene location at Chr 19: 19176903-19192591.

(SEQ ID NO: 128)
ACGCGGGGCGTCTGGAACTTAAAGGGGCAGCGTACCTTGGCCGTCCCGTT
CCACAACAAGGTCCCTTCTGCGGTCTCAAAGGCTTATAAGCAAGGGCGGG
GGCAGAGGGCTAAAGAAGGGAGTTTTTGCGGCCCGAGTGGGTAATGCGTG
GAGGACTCCCCCAACTCGTCTCTCAAGGAAAAGGGGGTTCAGAAAGGGAG
AGAGTTCTTCCCCCTCTCCTGCCTGGGTGTCGCTCCACCAGCCTCCTGGC
CGTCGCGCCCCCTCCTCACTGCGCCACCCCCGGGTGTGCCTCCTTCCCTG
CGTCTCACACAGCCCCTTTCTTGTCCGCAAAGACTGAGGAGCGGGGAGGG
CGGGCCCTCTGACACCGGAACAGGAGCCGTGCGGCGTCCGGTTTCAGCAG
GGAAGGCTTAGGCGGGCGTCCCAACCTCCGGGCTTCCGGTGCCGTTTCCC
GGTTCGCTCGGCCGCGGTCGCTATGGAGGAGCCGGAGATGCAGCTCAAGG
GGAAGAAAGTCACGGACAAGTTCACTGAGAGCGTCTACGTCCTGGCCAAC
GAGCCATCCGTGGCCCTGTACCGGCTGCAGGAGCATGTGCGTCGCTCCCT
CCCCGAGCTGGCCCAGCACAAGGCAGACATGCAGCGTTGGGAGGAGCAGA
GCCAGGGAGCCATCTACACTGTGGAGTACGCCTGCAGCGCCGTGAAGAAC
CTGGTGGACAGCAGCGTCTACTTCCGCAGCGTGGAGGGTCTGCTCAAACA
GGCCATCAGCATCCGGGACCATATGAATGCCAGTGCCCAGGGCCACAGGT
AGCTCCTGGGACCGCCCGCCCCCACTGCCACTCCCAGCTGCAAGGACCGT
CTCTCAGCTGCGCTGGGAACCGCTGCTTCTCGCTTATTAGAAAACTGTCT
CTTTCCTTTTGTCCTGGTGTCTGGTGACCTCCTCTTGTCCTGTGGCATCT
ATAACCTGAGTTCAGTCACTTAATACCGAGGTCCTGCGCTCTGCTGTGTG
CCTGGCCCTGGGCTGGGCACTGGGGACATAGCAGTGACCGAGACAGACAG
GCTCACAAGGAGACATACGACAACCAGGTAAACATGGCAGACAAGAGCAT
GTCAGATGCGCTGTGAAGAACACTGCGGGGCCCCTCCTAGGAGGTGGCAT
GAGTTACATGCAGACAGAGACGATCCGGGGGCAGACGGAGTTCCATGTGG
GGCAGTGGTGAGGGCAGACGCTCTGGGGCTGGGATCCCTGGGAGTGTTCG
AGAAGCACCGAGAAGGCTTCTGTGGCTGGAGCCGGCCAGCTGGGGGAGAT
GGGGCCAGGGAGATGGCAGGGGCCTCTCCCTGTCCCAGGACCCAGAGCCA
AGGGAGGCTTTAAGCCCAGGACCAGGGGTCTGAAAACGAAAAGCACTCAC
AGTCCTTGAACATTGGAAAGAGAATTTTTGAAGAGTAGCCCTTCTTTTTG
TTTTTGTTGGCATTCTAAGTTAATAAACACGCTTATGAA
(SEQ ID NO: 129)
MEEPEMQLKGKKVTDKFTESVYVLANEPSVALYRLQEHVRRSLPELAQHK
ADMQRWEEQSQGAIYTVEYACSAVKNLVDSSVYFRSVEGLLKQAISIRDH
MNASAQGHR

An amino acid sequence for the protein encoded by the CAND1 gene that is a regulator of T cell exhaustion/dysfunction resistance is available from the UniPROT database at accession no. Q86VP6, cDNA NM_001329674, with gene location at Chr 12: 67269358-67319953.

(SEQ ID NO: 130)
CTTTTGCCCTAGGGAGCGAGTGCGGAGCGAGTGGGAGCGAGACGGCCCTGAGTGGAAGTGTCTGGCTCCCCGT
AGAGGCCCTTCTGTACGCCCCGCCGCCCATGAGCTCGTTCTCACGCGAACAGCGCCGTCGTTAGGCTGGCTCT
GTAGCCTCGGCTTACCCCGGGACAGGCCCACGCCTCGCCAGGGAGGGGGCAGCCCGTCGAGGCGCCTCCCTAG
TCAGCGTCGGCGTCGCGCTGCGACCCTGGAAGCGGGAGCCGCCGCGAGCGAGAGGAGGAGCTCCAGTGGCGGC
GGCGGCGGCGGCAGCGGCAGCGGGCAGCAGCTCCAGCAGCGCCAGCAGGCGGGATCGAGGCCGTCAACATGGC
GAGCGCCTCGTACCACATTTCCAATTTGCTGGAAAAAATGACATCCAGCGACAAGGACTTTAGGTTTATGGCT
ACAAATGATTTGATGACGGAACTGCAGAAAGATTCCATCAAGTTGGATGATGATAGTGAAAGGAAAGTAGTGA
AAATGATTTTGAAGTTATTGGAAGATAAAAATGGAGAGGTACAGAATTTAGCTGTCAAATGTCTTGGTCCTTT
AGTGAGTAAAGTGAAAGAATACCAAGTAGAGACAATTGTAGATACCCTCTGCACTAACATGCTTTCTGATAAA
GAACAACTTCGAGACATTTCAAGTATTGGTCTTAAAACAGTAATTGGAGAACTTCCTCCAGCTTCCAGTGGCT
CTGCATTAGCTGCTAATGTATGTAAAAAGATTACTGGACGTCTTACAAGTGCAATAGCAAAACAGGAAGATGT
CTCTGTTCAGCTAGAAGCCTTGGATATTATGGCTGATATGTTGAGCAGGCAAGGAGGACTTCTTGTTAATTTC
CATCCTTCAATTCTGACCTGTCTACTTCCCCAGTTGACCAGCCCTAGACTTGCAGTGAGGAAAAGAACCATTA
TCGCTCTTGGCCATCTGGTTATGAGCTGTGGAAATATAGTTTTTGTAGATCTTATTGAACATCTGTTGTCAGA
GTTGTCCAAAAATGATTCTATGTCAACAACAAGAACCTACATACAATGTATTGCTGCTATTAGTAGGCAAGCT
GGTCATAGAATAGGTGAATACCTTGAGAAGATAATTCCTTTGGTGGTAAAATTTTGCAATGTAGATGATGATG
AATTAAGAGAGTACTGTATTCAAGCCTTTGAATCATTTGTAAGAAGATGTCCTAAGGAAGTATATCCTCATGT
TTCTACCATTATAAATATTTGTCTTAAATATCTTACCTATGATCCAAATTATAATTACGATGATGAAGATGAA
GATGAAAATGCAATGGATGCTGATGGTGGTGATGATGATGATCAAGGGAGTGATGATGAATACAGTGATGATG
ATGACATGAGTTGGAAAGTGAGACGTGCAGCTGCGAAGTGCTTGGATGCTGTAGTTAGCACAAGGCATGAAAT
GCTTCCAGAATTCTACAAGACCGTCTCTCCTGCACTAATATCCAGATTTAAAGAGCGTGAAGAGAATGTAAAG
GCAGATGTTTTTCACGCATACCTTTCTCTTTTGAAGCAAACTCGTCCTGTACAAAGTTGGCTATGTGACCCTG
ATGCAATGGAGCAGGGAGAAACACCTTTAACAATGCTTCAGAGTCAGGTTCCCAACATTGTTAAAGCTCTTCA
CAAACAGATGAAAGAAAAAAGTGTGAAGACCCGACAGTGTTGTTTTAACATGTTAACTGAGCTGGTAAATGTA
TTACCTGGGGCCCTAACTCAACACATTCCTGTACTTGTACCAGGAATCATTTTCTCACTGAATGATAAATCAA
GCTCATCGAATTTGAAGATCGATGCTTTGTCATGTCTATACGTAATCCTCTGTAACCATTCTCCTCAAGTCTT
CCATCCTCACGTTCAGGCTTTGGTTCCTCCAGTGGTGGCTTGTGTTGGAGACCCATTTTACAAAATTACATCT
GAAGCACTTCTTGTTACTCAACAGCTTGTCAAAGTAATTCGTCCTTTAGATCAGCCTTCCTCGTTTGATGCAA
CTCCTTATATCAAAGATCTATTTACCTGTACCATTAAGAGATTAAAAGCAGCTGACATTGATCAGGAAGTCAA
GGAAAGGGCTATTTCCTGTATGGGACAAATTATTTGCAACCTTGGAGACAATTTGGGTTCTGACTTGCCTAAT
ACACTTCAGATTTTCTTGGAGAGACTAAAGAATGAAATTACCAGGTTAACTACAGTAAAGGCATTGACACTGA
TTGCTGGGTCACCTTTGAAGATAGATTTGAGGCCTGTTCTGGGAGAAGGGGTTCCTATCCTTGCTTCATTTCT
TAGAAAAAACCAGAGAGCTTTGAAACTGGGTACTCTTTCTGCCCTTGATATTCTAATAAAAAACTATAGTGAC
AGCTTGACAGCTGCCATGATTGATGCAGTTCTAGATGAGCTCCCACCTCTTATCAGCGAAAGTGATATGCATG
TTTCACAAATGGCCATCAGTTTTCTTACCACTTTGGCAAAAGTATATCCCTCCTCCCTTTCAAAGATAAGTGG
ATCCATTCTCAATGAACTTATTGGACTTGTGAGATCACCCTTATTGCAGGGGGGAGCTCTTAGTGCCATGCTA
GACTTTTTCCAAGCTCTGGTTGTCACTGGAACAAATAATTTAGGATACATGGATTTGTTGCGCATGCTGACTG
GTCCAGTTTACTCTCAGAGCACAGCTCTTACTCATAAGCAGTCTTATTATTCCATTGCCAAATGTGTAGCTGC
CCTTACTCGAGCATGCCCTAAAGAGGGACCAGCTGTAGTAGGTCAGTTTATTCAAGATGTCAAGAACTCAAGG
TCTACAGATTCCATTCGTCTCTTAGCTCTACTTTCTCTTGGAGAAGTTGGGCATCATATTGACTTAAGTGGAC
AGTTGGAACTAAAATCTGTAATACTAGAAGCTTTCTCATCTCCTAGTGAAGAAGTCAAATCAGCTGCATCCTA
TGCATTAGGCAGCATTAGTGTGGGCAACCTTCCTGAATATCTGCCGTTTGTCCTGCAAGAAATAACTAGTCAA
CCCAAAAGGCAGTATCTTTTACTTCATTCCTTGAAGGAAATTATTAGCTCTGCATCAGTGGTGGGCCTTAAAC
CATATGTTGAAAACATCTGGGCCTTATTACTAAAGCACTGTGAGTGTGCAGAGGAAGGAACCAGAAATGTTGT
TGCTGAATGTCTAGGAAAACTCACTCTAATTGATCCAGAAACTCTCCTTCCACGGCTTAAGGGGTACTTGATA
TCAGGCTCATCATATGCCCGAAGCTCAGTGGTTACGGCTGTGAAATTTACAATTTCTGACCATCCACAACCTA
TTGATCCACTGTTAAAGAACTGCATAGGTGATTTCCTAAAAACTTTGGAAGACCCAGATTTGAATGTGAGAAG
AGTAGCCTTGGTCACATTTAATTCAGCAGCACATAACAAGCCATCATTAATAAGGGATCTATTGGATACTGTT
CTTCCACATCTTTACAATGAAACAAAAGTTAGAAAGGAGCTTATAAGAGAGGTAGAAATGGGTCCATTTAAAC
ATACGGTTGATGATGGTCTGGATATTAGAAAGGCAGCATTTGAGTGTATGTACACACTTCTAGACAGTTGTCT
TGATAGACTTGATATCTTTGAATTTCTAAATCATGTTGAAGATGGTTTGAAGGACCATTATGATATTAAGATG
CTGACATTTTTAATGTTGGTGAGACTGTCTACCCTTTGTCCAAGTGCAGTACTGCAGAGGTTGGACCGACTTG
TTGAGCCATTACGTGCAACATGTACAACTAAGGTAAAGGCAAACTCAGTAAAGCAGGAGTTTGAAAAACAAGA
TGAATTAAAGCGATCTGCCATGAGAGCAGTAGCAGCACTGCTAACCATTCCAGAAGCAGAGAAGAGTCCACTG
ATGAGTGAATTCCAGTCACAGATCAGTTCTAACCCTGAGCTGGCGGCTATCTTTGAAAGTATCCAGAAAGATT
CATCATCTACTAACTTGGAATCAATGGACACTAGTTAGATGTTTGTTCACCATGGGGACCATTACATATGACC
ATACAATGCACTGAATTGACAGGTTAATCATAAGACATGGAAAGAGAAGTGTCTAAAAGCTTCAAAATGTTCC
ACTTTTTTTTCCTTCATGGAGACTGTTTGTTTGGCTTTCTTCCATTGTTGTTTTTGTAGCATTTATTTCAGAA
ATGTGTATTTCCATAATCCAGAGGTTGTAAAACCACTAGTGTTTTAGTGGTTACAGCAACATTTGAAATGGAA
ACTAAAAGTTAGGATTTTATGGAGTATGGAGATAGGGTCCAGTATCTATTTACCCTGTAATGTTTAGGATTAA
AATGTTAAAATTTTGTGACCATGAATTTCTTTCTTTTATAAATTTTCTCATTTAAAAATCAAAAATCTTGCAA
AACAAAAACCATGTTTCTTTTTCTTGTATAACTTTTTGTTTTCAGCAACATAAATTGATTTTTAGCTGGCAGA
CAAGAATATCCATATAAGATTTGTTAACCATTTCAGAGAGTTTGGCAATTTTTAAAAGATAATAAGGTATCAT
TTTTAAGTATGAAAATTAACAATATCCCTGTTGCGCACACTAATTTTGCATGAGTAAGTTTACAAATATGTAT
CGTCTGTAAAGCAGCATGTGCAGATTATTCATAATATAGAAGTTAAAATAAGTATTAGTGCAATTTTCAGATA
TTTATTTTTGCACAGAAAACACATTATCTGGAGAGAAAGAAAGGAGAATTTTTGAGACTTGGGTTTTCTTAAT
GCCAGTGTGAATTTGCAGATGTTTTCAGAAAATCAAGTCACAGTAACAATTTGCCACTTTTTTCTATTATAAA
TCTTCTTACTTAAATTTTGAATATTTAGTTTTTCTCAGTTACCCATTTGTGTGTGTGTGATTCCACTTAGAAA
TTCTTAAAACCAGATTTTTCTTTCATTCCGTTTGGATGTCTACATTCCTTATCAAAGGATATAAATACTGTGT
ATGCTTTTGAATTTTATTTTTAGGAAAATTCTGAAGCCAGCTATCACAGGTTTGTTAGCTAATAATAGTATTT
TCTTTTAGTTGAGTTAGGTTTTTCCCCATCTCCTGTAGAGCGAATTTACATATTGTATTGGGTAAGTGTTCAC
TACTTTTCCTGATTAAGGGATCTGTGCTGGGGAACAAAGCTTTTGCAGTACCTTATATTGTAGTTAAAATTTT
ATTTAACATATCCTTCAGTGAGCTCATTTCACACTGTAGCCTCTTCCTTAAAATTTGTGGTGCTCCTGTAACA
GTAAGAACTAATTCTGAAATAAAAGACATCTCCTAATGCTGTGCAAACATAGTTTACATGTATTGAAGGAGGC
AGTTGTTAAATTGAGTGACCAATTTAAGCAATCAGATATTTGAAAACTGCACCCTTTAGTTTTGAAACTGTGA
ATTAGAAACACTTTTCCTGCTGTATTACTACCTGCTTTAACATCCAAATATACAGTGATTTTAAATGATAACA
TACTGTGGTTATTAGATTAACAGCTTGATTTTGAATGTTCAGATGATAATGCAGAAGACATCACTTCTAGTAA
GGATTTTGACTAGTGCATTGATGTTGAAGTTGGTGCCATTTCAAAATGTGGCAGGTGATAATCTTTTACCATA
ATTTGCATAAAACTGTAATAGAAGTTTATTTTGAGATGTTAGTATATTATGTACTATGCATTTCTGTGGTATA
GATGTTGTGGATATATTTAAGTATTTGGTTACATGGTTTTACAATAAATTACAATACTGCAGGCTCTAGGACT
GAACAGGAGACTGACATGCATATGTTGTGTGAATGTCTTAGTTGGGTAAAGTTAAATCCAAATACTTCAACTG
GCTTTTTCTTCAGTTTGTTTGTTTTTAACTTTCAAGATGCATGTTTTGTTTTGTTTTGCTTTGTTTTTGTCTC
TTAAGCCAATTCATTTTTCTAACAGTTGAAATTCTTTTAGTACTAAAGAAATAGATACTGCTCTTTTATGGCT
TTAGTGTTTATATACCTGTTTATATGCATACATTAACAAAATTAGTAACAGCAGTGCTTAGAATTTCAATGCT
GTGTCATACTGATGATTAACTCCATACTCCTAGTGGTGATGTGGTCCAGATACTGGAATGGAAGTGGCAGGAT
GTGGAGTCCAGACATAGTCTACCACTCATGCTACTTACTAGTAATATTAAGGTGTTAAACTTAGGAATTTGTC
ACATGTAGGTTCCTGAGGAGATCTAAAAGTTAATACAAAACCAAGCTGTTTACTGATTTTCATGAGAACATTT
AAGTTAGTTTAGGTCTACCTGATTTATTTTAAAAGAAGGCAGAGGGCTGGACGCAGTGGCTTACGCCTGTAAT
CCCAGCACTTTGGGAGGCCGAGGCGGGCGGATCACGAGGTCAGGAGATCGAGACTATCCTGGCTAACACTGTG
AAAACCCATCTCTACTGAAAATACAAAAAATTAGCTGGGCGTGGTGGCAGACGCCTGTAGTCCTAGCTGCTAC
AGCTGAGGCAGGAGAATGGCGTGAACCCAGGAGGCGGAGCTGGCAGTGAGCCGAGATCACGCCACTGCACTCT
GGCCTGGGCAACAGAGCGAGAGTCTGTCTCAAAAAAAAAAAAAAAAAAAAGGCAGGAAGGAAATTTGGTATGG
CAGGGCCTAACCTAAAAGGTTATTTATAAAATCAATAGCACTGAATGATAATTTTTTTAATGACAGTCATGTA
TTTTATTAATGATATCTGAAAGCTGCTTAACCATTAGGAATTACAATTCTTTATGGTTGGAGTATGATGATTT
CATCAAATGTTTGAATATTTACATAATTTTTATGTAACCATAATTAAACCAAATTAGTCTTTTTTTCTTCATA
CTTTTCTTCGGTAAATAGTGAAGAAATAATGGGCATATCTAATTAACCAGTTCTAGTGGGTGTTGTGATATCT
TTATATCTAGTTCAGTCTCAGATGTATGAATATGATTATATAGTCATGATTGAAGTTCAAAATGAGGAAGTAT
ACAAAATCCTCAGCCACTGAAAAATTCAGTTAACTTGTTTATACTCCTGTAAGCTCTAGTTAGTCACAAGACA
AAGTATATAGTAACACTTTGTAATGAAGTAACATTTTCTGCCCACATTAATTTTACTAAAGACCTAAATTGTT
AAAATTAATTGCCAATCTCTGGATATATTATGCTTAGTAAGTGTTTTGAAATTAAAGACCTTAAACATTTCAG
CAAGTAAATTTTGGATTCTAGCTCTGTTTTAAATTGAATCCTAATTTTCAAACCACTGCTACCCCAGATTCAT
ACCTGTTTTTGAAATCTTGTTTATGGAATGGAAAATTGACAGTATGGTAATAGTTTATTGACTGATTGGACAA
AGGATATTCTGGTTTTAACTGACTTTTCCATTTAAATAAAATTCATTTGATAATTTTGGTTGCAAAAGTGGAA
CACAGTGGAAGTATTTGGCTACAGGCTACCCAAATCTCATCAGATAAGGCTGTTTTCTTAAAGGAAATGCAAA
ATGAAATCATATGTTTTCCTTTTCCAATACAGTAAATTGCTTGTTTTCCTATGAATATCAGAAAAGATTTTGT
AAACACCTTTGCTGTAGTTTCAGGCAAGATCTTTCAGCAACATGTTTTTGCTCTTTGTAACTTAGAAAGTTAG
CTCTGAATCCAAAAACTACTGAAGTGAGTAATGCATATAATTTTACTTTTTTGGACCCTATAATGCCAGTGTG
TCAGGTTTCAGAATGATATTTAGTAGACAGAAATACCTAATTTTGTTTTGGAATCTTAATACCCTGTCCTCTT
CATAACTTTCTAGTGGTGATTTTAGCAAGTTTAATTATTTTGCTGAGGTCTTAGAAGACAGAAAGCTTATGCT
ATGTAAAGTGACATTGTTTCCAGATTAGGAAGTATTGGAATTAATTTGCTATTGCCAGTAAGTAATGGTTGAT
GGATATTAAGTGATTTTAGTCTAGAATGCTCTCAAAAAAATAGTAAAGAATTGTACTTCAGGGCCAGATGGCA
GCTCACGCCTGTAATCTCAGCACTTTGGGAGGCCAAGGTGGGGGATCACTTGAAGACAGGAGTTGGAGACCAG
CTCTAGGCAACATAGTGAGACCCTGTCTCTACCAAAAAGAAGAAAAAAGAAATCAACAACATGCAACCATAAG
AATGGATGAGATGGCCCTAAAAGAATAAGGATTACATATAATGAGAAGACGTGTGGGCTGAAATACCTAGTGA
ACAATATTTAAGGGAAACTGAAGTGCAAATGGCCTCACAGGTTTGTTTTTGTGTTTAAGACAAGGTCTCACTC
TGTTGCCTGGACTGGAGTACATAGCACGAACATGGTTTACTGCACCTCAGCTTCCTGGGCACAAGTGATTCTC
CCACCTCAGCCTCCCGAGTAGCTGGGAGTATAGGTGTGTGCCACCACGTCCAGCTAATTTTTTTATTTCCTTG
TGGAGATGGGGTCTCATGACCTTGTCCAGGATGATCTCAAACTCCTGGGCTCAAGCAATCCTGTGTTGGCCTC
CTAAACTGCTGAGATTATTGCAAGTGCCTCCTTGCCCAGCCACATGTTGTTGATTTCTTTTTTCTTTTTTTTT
CTTTCTTAATTTTTTTTTTTTTTTTTTTTTTTGAGATTGATGGAGTCTTGCTTTGTCTCCCAGGCTGGAGTGC
AGTGGCGCAATATCGGCTCACTGCAACCTCTGCCTCCCGGGTTCTAGCTATTCTCCTGCCTCAGCCTCCCAAG
TAGCTGTGATTACAGGCGTGTGCCACCATGCCCAGCTAATTTTTGTATTTTTAGTAGAGATGGAGTTTTGCCA
TGTTGGCCAGGCTGGTTTCAAACTCCTGATCTTAGGTGATACACCCGCCTCGGCCTCCCAAAGTGCTGGGATT
ACAGGCGTGAGCCACGTCCCCCAGCCAGTTTCTTAATCTATACATCTAGCCCAGTCCATGCCCCTGTTTCAGA
CTATTATATCCAACCACCTGCTATACATCTCATAAAGGTATCTCAAACACAGCATATCTAAAGCTGAAGTCAT
CATCTTGCCCTGGTTTTTCCTGTCTCTTTGCTGATGGCATCCCATTTACCCAGTCATTCAAACTGGAAATCTA
AAAGCCTCACTTAGCAAGAGTTTGGAGGCAGAATATGATAAAGAACAAGGACTCTGGGAGGCCAAGGCAGGTG
GATTGCTTGAGGTCAAGAGTTCGAGACCAGTCTGGCCAACATGGCGAAACCCCGTCTTTGCTAAAAAAACACA
AAGAAATTAGCTAGACGTGGTGGCACATGCCTGTAGTCCCAGCTGTGTGGGAGGCTGAGGCAGGGGAATTGCT
TGAACCTGGGAGGTGGAGGTTGCAGTGACCCAAGATTGCATGCACCACTGCACTCCAGCCTGGGTGACAAAGC
GAGACCCTGTCTCAAAAAAAACAAGGACTCTACAGATAGATAGACATCATATATCATATTAGTTTGAATCCTG
GCTGTTAGCTGTTAGACTTGGGCAAATAATTTAACCTCAACTTTCCCTATCTATAAAATGGGAATTAAAATAT
TTGCACCTTATAGGGTTATTGTGAGGATTAAATAGGAACATTGTATAAAGCACTTAAAACAGTACCTGGGACA
TACTAAGTAATATAATTGTTAACTCTTACTAGCATTCTGTCCCTTTTATTTCCTAAATATCTCTCATGCCCAT
CTCCTAAATATCTCTTTCCTAAATATCTCTCATGCTTTTGGCTTAAGACCTCTTTTCCTCCTTATCTATTGAC
TGGACTGCGGCAAATGCCTGCTAATCGTTATTTTTGTGTTCATTCATTTAGCAAGCCAATTTATTACGCGCTT
AGGGTGCTGCCAGGGCTACAAAAGCTGTTGAGACTGTACTTGATATGAAGAAGCTTGCTGATTATCATGGGAA
GACTGACATAAGGAAGCACCATAAAATTCTGCTGTATGAGAGGTATACACGGGATACTGGGGGTTAATAATTG
AGGGTATGGTCCATTCAACATGAGTGAGACAGAAACAATTCATAAAGGAGATGAAATGTCTTGAGGAATGAGC
TCTTAGAAGAATAGTTTTCAAATGAGTGTGCATCACAGTCACCTGTAAGACTTATTAAAACAGATCGCTGGGC
CCTACACCCAGAGGCTGTGGTTCAGTAGGCTGTAGTAAACCAGTAATTTGTATTTCTATGACGTTCCCAGGTT
CTAATGCTGTTCCCCAAGGCCACACCTTGGAAACCACCACATTAAAATACCCAGAAGGCATTAATTCCCAGTC
CTTCCTCTACACAGCTGCAAAACAATGGTCCTGACCATTTCATCTTTGCACTACATCCTTCACTGTCTCTCTT
TTGCCCATAGGATAAGTACAAACTAGATCTGGTTACTGCCTGCCCCACCAGCCTCAGCATCTCTCACAACTAG
GACTAACTTTTTCTTCTGACAACTATAAAATATTTCCCTTGCCTTCTCAAGTTTGCTCAAGGTCAAGTTATGC
CTTTTGCCTGGAATGACTTGACTTCTCTTTTGTTTTACTTAGCTGGCTGCTTTTCATCTTGTAGGTTAGGTCA
AGGACTCCAGGAAGTCTTCCCTGGACAAGTAATGAAGAGGGCATAATCCAAGGGCCAACTCCCATGTTTGGAA
CCTGACTCCATTTTCAGGCACGTAATATTGTCAAATTCCTTTTAAAAGCACCTGTCTGTCTGTTAACGTTGGT
GCAGATACTGCTATTCCCCTCCTCCATACCATTGCTGATGGTTACTGAGGGTATGGGAAGGGCCGACTAGTCC
AGCTGTTCACAAACAGCCCTTAATGTCAAACTGAATACTGCCAACGTAGTTCCAGTTTCTGTATCTAAAGACT
CAGCTTGGAGTCACTTGTCTGGACTAAAAGTAACCCCTCCTTGTCTGGTTTGTGACTTTCTGTACTCTGATGC
CCCCAGCTTTCTGCCTTCTAGAAATTTGTCAGAATTTCCAAAATTCTTGGGCCTTCCTTCTTGCTCTATATAT
GGTTTTGGATTCATTCCTTTTAAAAAATATTTACTGTCATTTCAGTAGAATTTTGACACAATAAATATAAGCA
CATCAGA
(SEQ ID NO; 131)
MASASYHISNLLEKMTSSDKDFRFMATNDLMTELQKDSIKLDDDSERKVVKMILKLLEDKNGEVQNLAVKCLG
PLVSKVKEYQVETIVDTLCTNMLSDKEQLRDISSIGLKTVIGELPPASSGSALAANVCKKITGRLTSAIAKQE
DVSVQLEALDIMADMLSRQGGLLVNFHPSILTCLLPQLTSPRLAVRKRTIIALGHLVMSCGNIVFVDLIEHLL
SELSKNDSMSTTRTYIQCIAAISRQAGHRIGEYLEKIIPLVVKFCNVDDDELREYCIQAFESFVRRCPKEVYP
HVSTIINICLKYLTYDPNYNYDDEDEDENAMDADGGDDDDQGSDDEYSDDDDMSWKVRRAAAKCLDAVVSTRH
EMLPEFYKTVSPALISRFKEREENVKADVFHAYLSLLKQTRPVQSWLCDPDAMEQGETPLTMLQSQVPNIVKA
LHKQMKEKSVKTRQCCFNMLTELVNVLPGALTQHIPVLVPGIIFSLNDKSSSSNLKIDALSCLYVILCNHSPQ
VFHPHVQALVPPVVACVGDPFYKITSEALLVTQQLVKVIRPLDQPSSFDATPYIKDLFTCTIKRLKAADIDQE
VKERAISCMGQIICNLGDNLGSDLPNTLQIFLERLKNEITRLTTVKALTLIAGSPLKIDLRPVLGEGVPILAS
FLRKNQRALKLGTLSALDILIKNYSDSLTAAMIDAVLDELPPLISESDMHVSQMAISFLTTLAKVYPSSLSKI
SGSILNELIGLVRSPLLQGGALSAMLDFFQALVVTGTNNLGYMDLLRMLTGPVYSQSTALTHKQSYYSIAKCV
AALTRACPKEGPAVVGQFIQDVKNSRSTDSIRLLALLSLGEVGHHIDLSGQLELKSVILEAFSSPSEEVKSAA
SYALGSISVGNLPEYLPFVLQEITSQPKRQYLLLHSLKEIISSASVVGLKPYVENIWALLLKHCECAEEGTRN
VVAECLGKLTLIDPETLLPRLKGYLISGSSYARSSVVTAVKFTISDHPQPIDPLLKNCIGDFLKTLEDPDLNV
RRVALVTFNSAAHNKPSLIRDLLDTVLPHLYNETKVRKELIREVEMGPFKHTVDDGLDIRKAAFECMYTLLDS
CLDRLDIFEFLNHVEDGLKDHYDIKMLTFLMLVRLSTLCPSAVLQRLDRLVEPLRATCTTKVKANSVKQEFEK
QDELKRSAMRAVAALLTIPEAEKSPLMSEFQSQISSNPELAAIFESIQKDSSSTNLESMDTS*

An amino acid sequence for the protein encoded by the CASP3 gene that is a regulator of T cell exhaustion/dysfunction resistance is available from the UniPROT database at accession no. P42574, cDNA NM_001354777, with gene location at Chr 4: 184627696-184650062.

(SEQ ID NO: 132)
AGTGCAGACGCGGCTCCTAGCGGATGGGTGCTATTGTGAGGCGGTTGTAG
AAGAGTTTCGTGAGTGCTCGCAGCTCATACCTGTGGCTGTGTATCCGTGG
CCACAGCTGGTTGGCGTCGCCTTGAAATCCCAGGCCGTGAGGAGTTAGCG
AGCCCTGCTCACACTCGGCGCTCTGGTTTTCGGTGGGTGTGCCCTGCACC
TGCCTCTTCCCCCATTCTCATTAATAAAGGTATCCATGGAGAACACTGAA
AACTCAGTGGATTCAAAATCCATTAAAAATTTGGAACCAAAGATCATACA
TGGAAGCGAATCAATGGACTCTGGAATATCCCTGGACAACAGTTATAAAA
TGGATTATCCTGAGATGGGTTTATGTATAATAATTAATAATAAGAATTTT
CATAAAAGCACTGGAATGACATCTCGGTCTGGTACAGATGTCGATGCAGC
AAACCTCAGGGAAACATTCAGAAACTTGAAATATGAAGTCAGGAATAAAA
ATGATCTTACACGTGAAGAAATTGTGGAATTGATGCGTGATGTTTCTAAA
GAAGATCACAGCAAAAGGAGCAGTTTTGTTTGTGTGCTTCTGAGCCATGG
TGAAGAAGGAATAATTTTTGGAACAAATGGACCTGTTGACCTGAAAAAAA
TAACAAACTTTTTCAGAGGGGATCGTTGTAGAAGTCTAACTGGAAAACCC
AAACTTTTCATTATTCAGGCCTGCCGTGGTACAGAACTGGACTGTGGCAT
TGAGACAGACAGTGGTGTTGATGATGACATGGCGTGTCATAAAATACCAG
TGGAGGCCGACTTCTTGTATGCATACTCCACAGCACCTGGTTATTATTCT
TGGCGAAATTCAAAGGATGGCTCCTGGTTCATCCAGTCGCTTTGTGCCAT
GCTGAAACAGTATGCCGACAAGCTTGAATTTATGCACATTCTTACCCGGG
TTAACCGAAAGGTGGCAACAGAATTTGAGTCCTTTTCCTTTGACGCTACT
TTTCATGCAAAGAAACAGATTCCATGTATTGTTTCCATGCTCACAAAAGA
ACTCTATTTTTATCACTAAAGAAATGGTTGGTTGGTGGTTTTTTTTAGTT
TGTATGCCAAGTGAGAAGATGGTATATTTGGTACTGTATTTCCCTCTCAT
TTTGACCTACTCTCATGCTGCAGAGGGTACTTTAAGACATACTCCTTCCA
TCAAATAGAACCACTATGAAGCTACCTCAAACTTCCAGTCAGGTAGTTGC
AATTGAATTAAATTAGGAATAAATAAAAATGGATACTGGTGCAGTCATTA
TGAGAGGCAATGATTGTTAATTTACAGCTTTCATGATTAGCAAGTTACAG
TGATGCTGTGCTATGAATTTTCAAGTAATTGTGAAAAAGTTAAACATTGA
AGTAATGAATTTTTATGATATTCCCCCCACTTAAGACTGTGTATTCTAGT
TTTGTCAAACTGTAGAAATGATGATGTGGAAGAACTTAGGCATCTGTGGG
CATGGTCAAAGGCTCAAACCTTTATTTTAGAATTGATATACACGGATGAC
TTAACTGCATTTTTAGACCATTTATCTGGGATTATGGTTTTGTGATGTTT
GTCCTGAACACTTTTGTTGTAAAAAAATAATAATAATGTTTAATATTGAG
AAAGAAACTAATATTTTATGTGAGAGAAAGTGTGAGCAAACTAACTTGAC
TTTTAAGGCTAAAACTTAACATTCATAGAGGGGTGGAGTTTTAACTGTAA
GGTGCTACAATGCCCCTGGATCTACCAGCATAAATATCTTCTGATTTGTC
CCTATGCATATCAGTTGAGCTTCATATACCAGCAATATATCTGAAGAGCT
ATTATATAAAAACCCCAAACTGTTGATTATTAGCCAGGTAATGTGAATAA
ATTCTATAGGAACATATGAAAATACAACTTAAATAATAAACAGTGGAATA
TAAGGAAAGCAATAAATGAATGGGCTGAGCTGCCTGTAACTTGAGAGTAG
ATGGTTTGAGCCTGAGCAGAGACATGACTCAGCCTGTTCCATGAAGGCAG
AGCCATGGACCACGCAGGAAGGGCCTACAGCCCATTTCTCCATACGCACT
GGTATGTGTGGATGATGCTGCCAGGGCGCCATCGCCAAGTAAGAAAGTGA
AGCAAATCAGAAACTTGTGAAGTGGAAATGTTCTAAAGGTGGTGAGGCAA
TAAAAATCATAGTACTCTTTGTAGCAAAATTCTTAAGTATGTTATTTTCT
GTTGAAGTTTACAATCAAAGGAAAATAGTAATGTTTTATACTGTTTACTG
AAAGAAAAAGACCTATGAGCACATAGGACTCTAGACGGCATCCAGCCGGA
GGCCAGAGCTGAGCCCTCAGCCCGGGAGGCAGGCTCCAGGCCTCAGCAGG
TGCGGAGCCGTCACTGCACCAAGTCTCACTGGCTGTCAGTATGACATTTC
ACGGGAGATTTCTTGTTGCTCAAAAAATGAGCTCGCATTTGTCAATGACA
GTTTCTTTTTTCTTACTAGACCTGTAACTTTTGTAAATACACATAGCATG
TAATGGTATCTTAAAGTGTGTTTCTATGTGACAATTTTGTACAAATTTGT
TATTTTCCATTTTTATTTCAAAATATACATTCAAACTTAAAATTA
(SEQ ID NO: 133)
MENTENSVDSKSIKNLEPKIIHGSESMDSGISLDNSYKMDYPEMGLCIII
NNKNFHKSTGMTSRSGTDVDAANLRETFRNLKYEVRNKNDLTREEIVELM
RDVSKEDHSKRSSFVCVLLSHGEEGIIFGTNGPVDLKKITNFFRGDRCRS
LTGKPKLFIIQACRGTELDCGIETDSGVDDDMACHKIPVEADFLYAYSTA
PGYYSWRNSKDGSWFIQSLCAMLKQYADKLEFMHILTRVNRKVATEFESF
SFDATFHAKKQIPCIVSMLTKELYFYH*

An amino acid sequence for the protein encoded by the CBFB gene that is a regulator of T cell exhaustion/dysfunction resistance is available from the UniPROT database at accession no. Q13951, cDNA NM_001755, with gene location at Chr 16: 67028984-67101058.

(SEQ ID NO: 134)
GGTGAGCGCTGGGGCTGCGCGGGCGGCAGGCAACGGCTGAGGCGGCGGCG
GCGGCGGCGGCGGCGTGGGTTGGGCTCGAGCGGGCGGCGGCGCCTCAGAC
TCCCCGGAACGGGAGCCCACGCGGGCGGGCGCCTGAAACAAAGGGAAGCG
GGCGTCCGGGCGCCGCGGGTGGGCGGTCAGTCGGTCAGCGCGGAGCCAGC
CAGCGGGTGCCCGCGCAAGCCCCGAGCGCGGCCGGCCGGCGCGGCCTCAG
GGCGGGAAGATGCCGCGCGTCGTGCCCGACCAGAGAAGCAAGTTCGAGAA
CGAGGAGTTTTTTAGGAAGCTGAGCCGCGAGTGTGAGATTAAGTACACGG
GCTTCAGGGACCGGCCCCACGAGGAACGCCAGGCACGCTTCCAGAACGCC
TGCCGCGACGGCCGCTCGGAAATCGCTTTTGTGGCCACAGGAACCAATCT
GTCTCTCCAGTTTTTTCCGGCCAGCTGGCAGGGAGAACAGCGACAAACAC
CTAGCCGAGAGTATGTCGACTTAGAAAGAGAAGCAGGCAAGGTATATTTG
AAGGCTCCCATGATTCTGAATGGAGTCTGTGTTATCTGGAAAGGCTGGAT
TGATCTCCAAAGACTGGATGGTATGGGCTGTCTGGAGTTTGATGAGGAGC
GAGCCCAGCAGGAGGATGCATTAGCACAACAGGCCTTTGAAGAGGCTCGG
AGAAGGACACGCGAATTTGAAGATAGAGACAGGTCTCATCGGGAGGAAAT
GGAGGTGAGAGTTTCACAGCTGCTGGCAGTAACTGGCAAGAAGACAACAA
GACCCTAGTCCTGGTTCCAATTTAGGTGGTGGTGATGACCTCAAACTTCG
TTAATTAATAGCACAGCAGATGTGTGCTGCCCATCTTTACATACACATTG
CTTCTAGTTGGCAGAAATAATTGATTAAAAGACCAGAAACTGTGATAACT
GGAGGTACTACGGTCTATTTCTCAACCTTAGGCAGTAATAGACATCACAA
ACTGCCATGGTTTTGCACTATGATTATAATACCTGCATTTCTAATTTTTT
AAGCATGTAGCCAGTAATAATTTGAAGTTTTTTTTCTATGCAAGCTTACC
TTGTTGGCATTATTTTAGGGAGTTGAAACTATCAACTGTAAAGCTCCTTT
TCTTCCACTTTAATTTAAAAGTTCATGTCATTTAAAAACAAGTCAAGAAA
TTAAAATTGTATCAGAGGGTTTTCTCTAATCATTTTTTCTATTTTTTTTT
TTGTACTTCTAGATGTTTTGGTTATACAGCTTCATTTTAGATGAGCATTC
TTATTTTTTGTTTTGTTTGCCCCATTTCCTTTTGTGTTTTTATAGTCTAT
AGCATTTTAAAACTGCTGATGTTGTTTGCATTATTTACAGGCTAAAAACT
TAGTAGCATAGAGCTGTCTGCCACAGCCTTCTGACAAAGTTTACAGTTAT
TAAAGTTGCAGTATCCTTTTAAATGCTAGTAATCAGCACTCTTTCTTTTT
TTTTTTTTTAATAGAGACAGGGTCTCGCAGTGTTGCCCAGGCTGGTCTCG
AACTCCTGGCATCAAGCGATCCTCCTGCCTTAGCCTCCCAGAGTACTGGG
ATTACAGGCTCTTTCTTTTTAAACATAAAAGTTTTAAATTGGTATTAACT
CTGTACTCTGCCCTAGATTGTTTTAGCTTCTGTTCTGTAATCATGAGTTT
GGTTGGAGATATTCTCCATAGATGATCTTCTACTGAAATGCCTAAAGAAG
TCACAGGCTGGCTTCTGTTTTATTCAGGGATTTTTTTAAAAAGTCAATCA
GAAAAGGGATACTGGAGCTTCTTCATGTATGTAACAGCATATTAAACTGG
AGACAGTGATGAATCAGCTACAAAGGTAATATTGTATTAAAATCATGTTT
AAGATAGCTGCTTTTATGTGTATTTTATATTGCATGCTTTTGTAAAAACA
TGCTGGGTGATGAAAGATTAGTTTTAGAGAGAAAATGTTCATCTGTGCAG
AGGATGCATTTTCTTCCATTAATTCTGGAAAAAACGTTCACAGTTATATA
TATGGTATTTTGCAAAAGGACTATTAATAGAACCTTTTGAGATGAATTAA
TGTAAGAATATTTTTTAAATAGGCTTACTGTCAAATTGCAACTTTTTTTT
TAGATACAGAGTGGAAAACAGTGCTAAGTCATTTGGCACCTCCTTACAAA
TATTTTTCATGGTCACATTTATTAAATGTTACTACATTTCTGAATTTTTG
AAAAATGTATTTTATCATTAAATGGCATTATTTTAAAGGGTGAAAAACTG
ACACAGTCAATTCAGAAAATGGACTGAAGTCTGAATAAGGTCATTGCATT
TAAAAAGCATATAACTGTACTTGACTGATGAGGGAGGTGTTACTTTCATT
GTATATAGGTCTTATTTCATAAACAGATATCCTGTATCAAATAAAAGTAT
TTGTTATATATTTGAAGTTATGCATGGAAAGGAGTGTGTTTAAATTGTTA
CAAACAATAATGCGTCATTAAAGGCCATGCTGATCTTGCATAACTATAAG
TACTATGAATGAATTTGGTTGGTTTTGGTGTTGTACAGCTCACATGTTTA
CACACTCAGTGCCCTAATTTCCCCTGAGGGAATCGCTTTTTAAGTGATCC
TTACAGTGGTGTTTTATGTTACTTTATTACAGAGCTCCTTGGTTTTTTAC
TTCTGCACTTAAATTTTTTTAAATAACATGATGATGGTACATTTTCCTCT
ATTGTCTAGCTAAGGGCTTTCGGTCCACCAGTAAATAAGATCAAATGCTC
TTAAATGTTCCTGTTACCATCCTAATGTAAATACTGGATTTTTCTGTCAT
TTAGCACCATGCTGCTTCTGTCTGTCTTAATGCTGGCATTAAGATCATGA
GCCCTTTTTCTCCAGTAGTACAGGCTTTGAAAACTACTTCTATTAAGTTA
TTGATGCAATTTGATATTTTTTCATAATCTATATTTAAACAAAATTACAT
CATTGCATCATCTTTTCTAAATTCATCTCCATTAAAACTTGCCTTAAGCT
ACCAGATTGCTTTTGCCACCATTGGCCATACTGTGTGTTTGTTTGTTTAA
TTTACTTTCACAATAAACTTCTGTGTAGTAAAAA
(SEQ ID NO: 135)
MPRVVPDQRSKFENEEFFRKLSRECEIKYTGFRDRPHEERQARFQNACRD
GRSEIAFVATGTNLSLQFFPASWQGEQRQTPSREYVDLEREAGKVYLKAP
MILNGVCVIWKGWIDLQRLDGMGCLEFDEERAQQEDALAQQAFEEARRRT
REFEDRDRSHREEMEVRVSQLLAVTGKKTTRP*

An amino acid sequence for the protein encoded by the CDK13 gene that is a regulator of T cell exhaustion/dysfunction resistance is available from the UniPROT database at accession no. Q14004, cDNA NM_003718, with gene location at Chr 7: 39950121-40099580.

(SEQ ID NO: 136)
ACTTCCGCGTTGCGCTGCCCGAGCCGAGAGCGCGGCCAAGGCCGCTCCCCCACCCCCGGGGGCACTTGGAGGA
CTCGGGACTCCCCCGCAGGTCAGCGCCCGGCGCATCTGGTGTTTTCGCTGCCGAGGATAGGACGACGAGCGCA
ATCGGGAGCTCCGCCGCCCGGATTCCTGCTTCCCTGGGGCCCGGAGGCTGCTGCGTACCCCACTGTGACCTGG
AACCCAGGGACCCGAGTCCCGACCCGGATTATCGTGGCGCTTTTCCCGGCCGGCTCTGGTGCTCGGTGTCCCT
CCGCCGCCGCTCCCGTTTCCGGCGGGGGAGATGGCCAGGATCTGACCCGGGAGGAGGCCGCACCCGCGCCGCG
CTCTGCGGCTGGCTCTAGGCGATGCCGAGCAGCTCGGACACGGCGCTGGGGGGAGGCGGGGGCCTGAGCTGGG
CGGAGAAGAAGTTGGAGGAACGCCGCAAGCGGAGGCGATTCCTGTCCCCTCAGCAGCCGCCGCTGCTGTTGCC
GCTCCTGCAGCCGCAGCTCCTGCAACCGCCGCCGCCCCCGCCGCCTCTGCTCTTCCTGGCTGCTCCCGGCACG
GCCGCCGCCGCAGCCGCCGCCGCCGCGGCCTCCTCCTCTTGCTTCAGCCCGGGCCCCCCTCTGGAGGTCAAGC
GGCTGGCGAGAGGCAAGAGGCGCGCAGGAGGGCGGCAGAAGCGGCGTCGCGGGCCCCGCGCCGGGCAGGAGGC
GGAGAAGCGTCGGGTCTTCTCGCTGCCCCAGCCGCAGCAGGACGGCGGTGGCGGTGCTAGTAGCGGCGGGGGT
GTGACCCCGCTGGTGGAATACGAGGATGTGAGCTCCCAGTCCGAGCAGGGGCTGCTGCTGGGGGGGGCCAGCG
CGGCAACGGCGGCGACGGCTGCCGGGGGAACGGGGGGCAGCGGCGGGAGTCCGGCCTCCTCCTCCGGCACCCA
GCGGCGCGGGGAGGGGTCGGAGCGCAGGCCCCGCCGGGACCGCCGCAGCAGCAGTGGCCGCAGCAAGGAGCGC
CACCGCGAGCACCGGCGGCGGGATGGGCAGCGCGGTGGCAGCGAGGCCTCCAAGTCCCGCAGCCGCCACAGCC
ACAGCGGCGAGGAACGGGCCGAGGTCGCCAAGAGCGGCAGCAGCAGCAGCAGCGGCGGCCGCCGGAAAAGCGC
TTCGGCCACATCCAGCAGCAGTAGCAGCCGCAAGGACCGGGACTCGAAGGCCCACCGCAGCCGGACTAAGTCG
TCCAAGGAGCCGCCTTCGGCCTACAAGGAACCGCCCAAGGCCTACCGGGAGGACAAGACCGAGCCTAAGGCCT
ACAGGCGGCGGCGGTCCCTCAGCCCACTGGGAGGCCGGGACGACAGCCCGGTGTCCCACAGGGCCTCTCAGAG
CCTGAGGAGCCGCAAGTCCCCCAGCCCGGCAGGAGGTGGCAGCAGCCCCTATTCTCGGCGGCTGCCGCGCTCC
CCGAGCCCCTACAGTCGCCGCCGCTCCCCCAGCTACAGCCGCCACAGCTCCTACGAGCGGGGCGGCGACGTGT
CCCCTAGTCCCTACAGCAGCAGCAGCTGGCGCCGCTCTCGCAGTCCCTACAGCCCTGTGCTCAGACGGTCTGG
AAAATCCCGAAGCAGAAGCCCGTATTCATCTAGGCATTCAAGATCTCGTAGCAGGCACAGATTGTCTAGATCC
AGAAGTCGTCATTCTAGTATTTCTCCTAGCACACTAACTCTGAAGAGTAGCCTGGCAGCTGAATTGAACAAGA
ATAAAAAAGCACGAGCAGCAGAGGCAGCAAGAGCCGCAGAAGCAGCGAAAGCTGCAGAAGCAACTAAGGCTGC
TGAGGCTGCTGCCAAGGCTGCAAAAGCTTCAAACACTTCTACACCTACCAAGGGGAACACGGAAACTAGTGCC
AGTGCATCACAAACAAACCATGTGAAGGATGTGAAGAAAATTAAAATTGAACATGCACCTTCTCCCTCAAGTG
GTGGAACTTTAAAAAATGACAAAGCAAAAACAAAGCCACCTCTTCAGGTAACGAAGGTGGAAAATAATTTGAT
TGTAGATAAAGCCACCAAGAAAGCAGTCATAGTTGGAAAGGAGAGTAAATCTGCTGCTACAAAGGAGGAATCA
GTATCTCTTAAAGAGAAAACCAAACCACTTACACCAAGCATAGGAGCCAAGGAGAAGGAGCAACATGTAGCTT
TAGTCACCTCTACATTACCACCGTTACCTTTGCCTCCCATGCTGCCTGAAGATAAAGAAGCTGATAGCTTACG
AGGAAATATTTCAGTAAAAGCAGTTAAAAAAGAAGTAGAAAAGAAACTCCGATGTCTTCTTGCTGATTTACCG
CTGCCCCCTGAGCTACCAGGAGGAGATGATCTTTCAAAGAGTCCAGAGGAAAAGAAAACAGCAACACAGTTAC
ATAGTAAAAGGAGGCCTAAAATATGTGGGCCTCGCTATGGTGAAACCAAAGAAAAAGATATTGACTGGGGAAA
ACGCTGCGTGGATAAATTTGATATCATCGGAATTATTGGAGAAGGTACTTACGGACAAGTTTACAAAGCCAGG
GATAAAGACACTGGAGAAATGGTAGCCTTAAAAAAAGTACGTCTGGATAATGAAAAGGAAGGCTTTCCAATTA
CAGCAATTCGAGAAATTAAAATTCTCCGGCAGCTTACCCATCAGAGTATTATCAATATGAAGGAAATAGTGAC
TGATAAAGAAGATGCTTTGGATTTCAAGAAGGACAAAGGTGCATTTTATCTGGTGTTTGAATATATGGACCAT
GATCTGATGGGACTACTGGAATCAGGCTTGGTTCATTTTAATGAAAATCACATAAAGTCATTTATGAGACAGC
TCATGGAGGGTCTGGATTATTGTCATAAGAAGAACTTTTTGCATAGAGATATTAAATGTTCCAATATCCTTCT
AAATAATAGAGGGCAGATAAAACTTGCAGACTTTGGACTTGCTCGATTGTATAGCTCAGAAGAAAGTCGGCCG
TATACTAACAAGGTAATTACTTTATGGTACCGTCCACCTGAACTGCTACTGGGAGAAGAACGATACACACCAG
CCATTGATGTATGGAGCTGTGGCTGTATCCTTGGCGAACTCTTCACTAAAAAACCTATATTTCAAGCAAATCA
GGAACTTGCACAACTAGAATTAATAAGCCGAATATGTGGGAGTCCATGTCCTGCAGTGTGGCCTGATGTAATC
AAACTACCATATTTCAACACCATGAAACCAAAGAAGCAATATCGTCGAAAGTTAAGAGAAGAATTTGTTTTTA
TTCCTGCAGCTGCGCTAGACTTATTTGATTACATGCTTGCCTTGGATCCTAGTAAGCGCTGCACTGCTGAACA
GGCTCTTCAGTGCGAGTTCCTCCGAGATGTGGAACCCTCAAAAATGCCTCCACCAGATCTCCCTTTATGGCAA
GATTGTCATGAGTTATGGAGTAAAAAGCGAAGAAGACAGAAGCAGATGGGCATGACTGATGATGTTTCCACAA
TTAAAGCCCCCAGGAAGGACTTGTCTCTGGGCTTGGATGACAGCAGAACCAACACACCCCAGGGTGTGCTGCC
ATCTTCACAGCTGAAATCTCAGGGCAGCTCAAATGTGGCACCTGTAAAAACAGGCCCTGGACAGCACTTAAAC
CACAGTGAATTGGCAATTCTACTAAACCTACTACAATCTAAAACAAGTGTTAATATGGCTGATTTTGTCCAAG
TGTTGAACATTAAGGTAAACTCTGAGACTCAACAGCAGCTAAATAAAATAAACCTTCCTGCTGGAATTTTGGC
AACAGGTGAAAAACAGACAGATCCATCAACACCACAACAGGAGTCTTCGAAACCGTTGGGAGGAATTCAGCCT
TCTTCTCAGACCATCCAGCCTAAAGTGGAGACTGATGCTGCCCAGGCGGCTGTGCAGAGTGCATTTGCAGTTC
TGTTGACTCAGTTAATAAAGGCTCAGCAGTCAAAGCAGAAAGATGTGCTACTAGAAGAGAGGGAAAATGGATC
GGGACATGAAGCGTCATTACAACTCAGGCCACCTCCAGAACCTAGCACTCCGGTGTCGGGACAAGATGACCTC
ATCCAGCATCAAGATATGAGGATCTTGGAGCTAACGCCAGAACCAGACCGGCCTCGAATTCTGCCTCCTGACC
AACGACCTCCCGAGCCTCCTGAACCACCACCAGTCACTGAGGAAGATCTAGATTATCGGACAGAAAACCAGCA
TGTACCCACCACCAGTTCTTCATTAACTGACCCTCATGCCGGAGTGAAGGCAGCCCTGTTACAGCTGCTTGCT
CAGCATCAGCCCCAGGATGACCCCAAAAGAGAAGGTGGGATTGATTATCAAGCAGGAGACACTTACGTGTCCA
CTTCAGACTACAAGGACAACTTTGGATCCTCTTCTTTCTCTTCTGCTCCTTATGTTAGCAATGATGGTCTAGG
AAGCAGTTCTGCTCCACCACTAGAACGACGTAGTTTCATTGGAAATTCAGATATTCAGTCTTTGGATAACTAC
AGTACTGCTTCATCTCATTCTGGTGGTCCACCTCAGCCTTCTGCCTTTTCTGAGTCATTTCCCAGTTCAGTAG
CTGGATATGGAGACATTTACCTCAATGCTGGTCCCATGTTGTTTAGTGGAGACAAGGACCATAGATTTGAATA
TAGCCATGGTCCTATTGCAGTCCTGGCAAACAGCAGTGACCCTTCCACGGGGCCAGAGAGTACTCATCCTTTG
CCAGCAAAGATGCACAACTATAACTATGGTGGTAACTTACAGGAAAATCCGAGTGGCCCCAGCCTCATGCATG
GACAGACCTGGACTTCTCCTGCCCAAGGACCTGGATATTCACAAGGATACAGGGGACATATTAGCACATCAAC
TGGCAGAGGCAGAGGCAGAGGGTTACCATACTGAGTATCTGTTTTTCCTCAGGCACATCATTTTTATCTGGAA
AGACTTTTCTAGCTGCAATTTAAGGCAGCAATCCAAGAGACTTGAATAATAATAATTCAACAACAGCTTTATT
TTTATGTGGAGAAGGGTCTTGCATACAATAGTTTAAAAAAGACAAAAAAAACCTTTGCTTAAATTCATGCTGT
TCTAAAAACTAGATCGATTGTACATCTTCACAAATTCTAGTTAACAATTTTATTTTGTATTCTTGCAGTTTTA
AGTGGATGCTAATTTTAGGGGCATAAGCCTTTTATGGCCCTCTTGCAGATCTTCTGAACTATGCACATTTGTG
CTTTTTTTGTAAGTTTGGACCAACTTTTATGTAACAAACAGCCCCTCCCCACCTCCAGTTTTACAACAATCAG
AAAGGGCACTGATTTATTTGGTATTTTTCTTTTTACAAAGCTACCTTTAGTCAAAGGTCACTGTCAGTCTTTG
CACCTGCTTTCAGTGTTATTGTGAAAGGTGTACTTTGTGCTCATTTCAGAAAATAAAACACAACCTTTCTCTT
GATGCAACAGTTTTATAAAAAAAAAAATGGTCAACGTTATTTTTGTTTTGTTTTGCAGATTATCAAAGCCTAG
CAAAATGCATTTAAATGAAATAGTGGGTTTTATATGAAAACTATGGGTGGGGTGGGGAGGGAAAGTAAGTGCC
TTAACAGGTAAGCTTAAGGTCTGAAAAAAATAGTTAACTTTTACCCCCATTTGTCTTTTAAAGGGAATCAATG
CATTAAAAAGGCAAGAAACTCTGAAGTTTCAGGGGCTGCCTGTTCAGTTCCTGTTAGGTCCATTCCCTCTGCT
TGTCACACTTTCTCCTTTTCTGCCTGTCTTTGGGCTGTGTTGCCTTTCACTACCACCTTCTCTTGATAGGGGA
GTGAGAGCAAAGAAAGGGAGTAGAACTGCAATCAGTACATATTGTTTGAGAAGTTCTACAAAGTTTTGTCATT
TAAATGGTTGAAAGTAATAGCTCCAGGAGAATTTTAGAGTACCATTTTCTTTCAGGCTGCCATATTATCCCTG
TTTCCATTTGCCATGGATAATTGGAGGGTCATGGTATAGAAGCAAGAGTTAATAACAGAACTTGCCTAAATGT
GTTAAAGTTCATGGGGGAGGGGTGGCAAATACCGTATTTTAAGTTATGCAATTGCCCTTTTAAGAAAGTCAAT
TCTATTCACTTGTTAACTAGAAACATTGCTTAGATTGAATTTATTGAAGCAAAAAGAAAAGGCAATGAGAGAG
AACTTTTATCTTCCTTCCTGTCACATTCTGTAAAGTTGATTATGACTTCTGCCAGACTTAGAGCAACAACTTT
AGTTCATTTTAACATTTTTTTTTAATGAAGAGCATCAAAGAATGGAGTAGTCCTTATTTAAACTGTAATGGTG
TCAAGTCTCACTGTGTGCACACTCTCACACATACACACACAATTATACTAATTCTAGAAAGGAACCAAAGGTA
AATAACCAGTGTTTCCATTATCACTAAATGGAAAAATTGACTGGAAGTTAGAGTTGTGCCAATTAATCATTTT
GTTCATCAACTGTTCTGGTAACTTATTTTTTTTTATTGGAATCTTAAATTGGAACAACTGTTACAGAATGTAT
ATTGCTCCAGTTCTAAAGCCAGTTAACCCTGTGCCCTCTAACTTATGAACCTTCATTTCAAAACAGTATGTGG
GAAGTCCTGTAGTTTGATAAGAGTAAAATTATTTTAAAGAGGATAATTTACCATTTTACTACTATAAAATAAG
TTGATATCAGTTGACCATTTTATTTTTTGATGGACTCTTTTTGTTGGGAGAAAGAAATGAATGGAAGAAAAAA
ATATGTTGCTTTTATTTGAATATTGATTACTAACAACTTATAAACTACATTGCAGGAAATGAAGAAATATTTT
GAAATTAGCAAAATAGCAACAGTATTCAAGTATCTGAAATTTTAATATGGTGAGTCTTAGAGCTTTAGACTTT
CCTATAAGTGACAGTATCTAAGTTATTCTTAATAGTCACACTTGATAATTCTGCTGACCATAAGCAGCTAACC
TACAGTTGAAGATATTTATGTGACATGAATAGCCTGTGTTTTAAAAATTAAATATTTTATATAAAATTGAACT
GTGTTTGGATAGTTTTTCTAATCAAACATTTCAGTTATATATTTATGTTGGGAGAGTGGCTGGGAGGACAGGG
AAATTGTTGCTTTGACATTGAAAATTTGAACTAGAACTTCTAATATTATCCTTACACAGCACTACTGTGAGAC
ATACTAGTCTTTTATATGTCACTTAGAAGAAAAAATGTTGCCAATTAAGCCACAATACAATGCTAAGACGTCA
TCAATTGTAAGCTGCATCCCAGATTCAGACATATTATATGAAAATATGTCTTATAATTGATGAAATATGATAG
TAAAAAATTTTAATGCTTCCCTTCATAGCATTTAATATTTTAATTCAGTGAATTTGGATTAATAGAACAAAGT
TGGGAAATCACTAGTTCTGTAGGTTATAACAGCAGTCATCGCTGAAAATTTGAGTGAAATCTAAAGAACACAA
AATATTGCTTAAATTTTTTGGAAAAAAGCAGGTAAGTCAGACTGCAAGTTAAGAAGACAAAAATATAAAAGAT
TTGACCTGCCAAAAGAAGAAAGTATTTATCTTCTCCCACGTGAAATTCCCTTTTGTATTTACTGACACAGCAG
GTTTTTCTGCATGCAACTCAGTATGTTTCCTACGTCTTCATTATTTGTTGGAGAGTTGCAAGTCATTTGTGAA
ACCTTAAAATGCCAATTTTAAGGAGTTGTCATTTGTGGAATGTGAAATAAGGTAATAATTCTAGCTGTGATTC
AGAGCTTTATTCTGTCAGCAAATACATAGTCAGTGCCTACAACATGCCAGGTAGTAATCTAGTCCTTGGGGTA
CATCAATAACCGTCAACAGTGTTAAATCATACTTTCAGTGTGCTGACAGTGAGCAAGATTTTTTTAAATGTCT
GGAGAAGTTAGTGGTATTTGCTACCCTCAGCTGTATCTAAATTGCACTTAAGTGAATGTAGGGCATGAAAATT
TGAGTATCTTTTTGAAATTTTAAATTGAAATTTGGATAGAGATGGTTATGGAGAGAAATCAAACAACTGGAAT
AGCTGTTTGATATCACTTAAAAGTGATAAAATTTTAAGTTGAATCTGGTCAGTTTGCAATGGCCTATTTGTAA
GAAATATCAAGACTTCTTGAGAAAAATGAAAAGTGAATACATAAATGCTTAAAATCTGGTACTTCTGAGTTAA
GGTTTTGCTCTTTGAGCTTAATCCAATTTGGGATGATTTTTCATCCTAGGGCTTTTTGTTTTCCTTTTTTATT
TTTATTTTTTCTTTTTTTAGGGGAAGGGGACTTGCTTTCTTTTCCAAAAAGGTGAATCCTTCTTGTAGGACAT
AGGTAAAAAAAACAAAGCTGAAATATATGTTTTGAATATAGATAGCTAATTCCCTGGGATATAATATCCTTTC
AATTTTTTTTTTTTTTTTGGCCCAGTCTGCCTTTTGATGTTTTAAAAGTCTGAACGAGATGTCCCAGTAACCT
AAAATTATCCAGTCGGTCTTCTTACTTTACAACTAAGAAAAATAAGGCTTAGAAAGAGGGATTGCCAGAAACT
TTGGCAGCTGGATTGCCTGTGCTTGTTCCTCTAAGCCATACCTAAATTCTGCAGTAAATACTTAACTTTTTAA
TAGGGAAATTGCTTCAAGATAACTTGACCAGTGATACGGTAAAATAATTAGACTATTGGACTAATGGTTTAAC
ACAAGTGGCTTTAAAAAGTCTGCTTAAAAAACAATTTTTATTTAGAAAAAATAGAAAAATAAAAACATCTTCA
AAATTTAGGAGCCTGAAGGGGCTGTTTGTTTCATATATGGATAATCTTTGAAAAGGCAAGTCCTGTATGTATT
TTTCATTTGTTGAAAGAAGATTGGTTATCAGTAGGCTTGCAAACATAATTTGCTTTTAAGTTCTTTCAAGGTT
TTATGCAATAAAACCTATTGATTTGGAACTTTAAAAAAAAAAACAACAAAAAAATACTTTCAGGGTTTTGTAA
TTTCAAGTGGTTTTTTAAGGGGAGCAATAGTTTGCCATTTACCAAAGGCTTCTCCAGATAATTTCTTAAATGT
TTCTACTTAAAAATAAAAGCTATTAATAATAAGCTGTCATGGGATCCATTTGAAGACAGGGAAAATAGAAAAT
TTTTATTGTAAAGGGAAGAACTTATCCTTTTAATTTTATGGACTAACAGAGTCTGCAGGTCTTAACTCATTTC
AGCCTGTCAAATGTGCAATTAAAAATGAATTTTCTAATTGTATTCAAATGAGGCTCTATAGTGAATACAGAAT
CACTCTTCTAAGTTTTTTCCCAGTTAATTTGTTTAAAAGTGTTGTACTCTCTTGCAAGAACGTTTAAAAGTTA
AGTCTTGTAACTGTTAACATCTAATGTATTAATATAAGCCATTTGTTTTTTACCATTTTTTTAAGGCCGTATT
CAATAGTCTGTAAATAAATTGCCGTAACACTCCCA
(SEQ ID NO: 137)
MPSSSDTALGGGGGLSWAEKKLEERRKRRRFLSPQQPPLLLPLLQPQLLQPPPPPPPLLFLAAPGTAAAAAAA
AAASSSCFSPGPPLEVKRLARGKRRAGGRQKRRRGPRAGQEAEKRRVFSLPQPQQDGGGGASSGGGVTPLVEY
EDVSSQSEQGLLLGGASAATAATAAGGTGGSGGSPASSSGTQRRGEGSERRPRRDRRSSSGRSKERHREHRRR
DGQRGGSEASKSRSRHSHSGEERAEVAKSGSSSSSGGRRKSASATSSSSSSRKDRDSKAHRSRTKSSKEPPSA
YKEPPKAYREDKTEPKAYRRRRSLSPLGGRDDSPVSHRASQSLRSRKSPSPAGGGSSPYSRRLPRSPSPYSRR
RSPSYSRHSSYERGGDVSPSPYSSSSWRRSRSPYSPVLRRSGKSRSRSPYSSRHSRSRSRHRLSRSRSRHSSI
SPSTLTLKSSLAAELNKNKKARAAEAARAAEAAKAAEATKAAEAAAKAAKASNTSTPTKGNTETSASASQTNH
VKDVKKIKIEHAPSPSSGGTLKNDKAKTKPPLQVTKVENNLIVDKATKKAVIVGKESKSAATKEESVSLKEKT
KPLTPSIGAKEKEQHVALVTSTLPPLPLPPMLPEDKEADSLRGNISVKAVKKEVEKKLRCLLADLPLPPELPG
GDDLSKSPEEKKTATQLHSKRRPKICGPRYGETKEKDIDWGKRCVDKFDIIGIIGEGTYGQVYKARDKDTGEM
VALKKVRLDNEKEGFPITAIREIKILRQLTHQSIINMKEIVTDKEDALDFKKDKGAFYLVFEYMDHDLMGLLE
SGLVHFNENHIKSFMRQLMEGLDYCHKKNFLHRDIKCSNILLNNRGQIKLADFGLARLYSSEESRPYTNKVIT
LWYRPPELLLGEERYTPAIDVWSCGCILGELFTKKPIFQANQELAQLELISRICGSPCPAVWPDVIKLPYFNT
MKPKKQYRRKLREEFVFIPAAALDLFDYMLALDPSKRCTAEQALQCEFLRDVEPSKMPPPDLPLWQDCHELWS
KKRRRQKQMGMTDDVSTIKAPRKDLSLGLDDSRTNTPQGVLPSSQLKSQGSSNVAPVKTGPGQHLNHSELAIL
LNLLQSKTSVNMADFVQVLNIKVNSETQQQLNKINLPAGILATGEKQTDPSTPQQESSKPLGGIQPSSQTIQP
KVETDAAQAAVQSAFAVLLTQLIKAQQSKQKDVLLEERENGSGHEASLQLRPPPEPSTPVSGQDDLIQHQDMR
ILELTPEPDRPRILPPDQRPPEPPEPPPVTEEDLDYRTENQHVPTTSSSLTDPHAGVKAALLQLLAQHQPQDD
PKREGGIDYQAGDTYVSTSDYKDNFGSSSFSSAPYVSNDGLGSSSAPPLERRSFIGNSDIQSLDNYSTASSHS
GGPPQPSAFSESFPSSVAGYGDIYLNAGPMLFSGDKDHRFEYSHGPIAVLANSSDPSTGPESTHPLPAKMHNY
NYGGNLQENPSGPSLMHGQTWTSPAQGPGYSQGYRGHISTSTGRGRGRGLPY*

An amino acid sequence for the protein encoded by the CDKN2A gene that is a regulator of T cell exhaustion/dysfunction resistance is available from the UniPROT database at accession no. P42771, cDNA NM_000077, with gene location at Chr 9: 21967752-21995301.

(SEQ ID NO: 138)
GAGAGCAGGCAGCGGGCGGCGGGGAGCAGCATGGAGCCGGCGGCGGGGAG
CAGCATGGAGCCTTCGGCTGACTGGCTGGCCACGGCCGCGGCCCGGGGTC
GGGTAGAGGAGGTGCGGGCGCTGCTGGAGGCGGGGGCGCTGCCCAACGCA
CCGAATAGTTACGGTCGGAGGCCGATCCAGGTCATGATGATGGGCAGCGC
CCGAGTGGCGGAGCTGCTGCTGCTCCACGGCGCGGAGCCCAACTGCGCCG
ACCCCGCCACTCTCACCCGACCCGTGCACGACGCTGCCCGGGAGGGCTTC
CTGGACACGCTGGTGGTGCTGCACCGGGCCGGGGCGCGGCTGGACGTGCG
CGATGCCTGGGGCCGTCTGCCCGTGGACCTGGCTGAGGAGCTGGGCCATC
GCGATGTCGCACGGTACCTGCGCGCGGCTGCGGGGGGCACCAGAGGCAGT
AACCATGCCCGCATAGATGCCGCGGAAGGTCCCTCAGACATCCCCGATTG
AAAGAACCAGAGAGGCTCTGAGAAACCTCGGGAAACTTAGATCATCAGTC
ACCGAAGGTCCTACAGGGCCACAACTGCCCCCGCCACAACCCACCCCGCT
TTCGTAGTTTTCATTTAGAAAATAGAGCTTTTAAAAATGTCCTGCCTTTT
AACGTAGATATATGCCTTCCCCCACTACCGTAAATGTCCATTTATATCAT
TTTTTATATATTCTTATAAAAATGTAAAAAAGAAAAACACCGCTTCTGCC
TTTTCACTGTGTTGGAGTTTTCTGGAGTGAGCACTCACGCCCTAAGCGCA
CATTCATGTGGGCATTTCTTGCGAGCCTCGCAGCCTCCGGAAGCTGTCGA
CTTCATGACAAGCATTTTGTGAACTAGGGAAGCTCAGGGGGGTTACTGGC
TTCTCTTGAGTCACACTGCTAGCAAATGGCAGAACCAAAGCTCAAATAAA
AATAAAATAATTTTCATTCATTCACTCA
(SEQ ID NO: 139)
MEPAAGSSMEPSADWLATAAARGRVEEVRALLEAGALPNAPNSYGRRPIQ
VMMMGSARVAELLLLHGAEPNCADPATLTRPVHDAAREGFLDTLVVLHRA
GARLDVRDAWGRLPVDLAEELGHRDVARYLRAAAGGTRGSNHARIDAAEG
PSDIPD*

An amino acid sequence for the protein encoded by the CFAP54 gene that is a regulator of T cell exhaustion/dysfunction resistance is available from the UniPROT database at accession no. Q96N23, cDNA NM_001367885, with gene location at Chr 12: 96489571-96875555.

(SEQ ID NO: 140)
ACACATACTCCAGGCGGGCCGGGGCGCGTCAATATGGCGGCGCAGGGCTCCCCCTCGAGCTCTCCGTCAGACG
ACTCTACCACCTCGGGGTCTCTGCCAGAACTGCCGCCGACCTCCACCGCGACTTCGAGGTCGCCCCCAGAGTC
GAAGGGGAGCTCCCGGAGCTCGCTGCTTCAGTGGACCTGCCCCGAGGACTCATTGCCCCTAGCCGTGTTTTAT
GGGCCGCTGGACGCGAAAAACCCGCTCCTGGCCTCTTGTGAGAAGGAGATCCAGGAGTTGTTAGGCTTTATGA
GGAAAAAGAAGGCTTTAGCCACCACGGAGGAAGAGAAGCACGAATTCCGCCGGCGTTGTGCCACTTCTTTGTT
TAATATCTGGACTAAATACGCCCCCAGGCTGCCAGCAGACTATTACAACGAAAAGCTTCTGAAGGTTGGAGAT
AGCCTTTGTCAAATGAAAGAATACAAACTGGCCCTTTTACAATGCTATGGAAGATATCTTCAGCAGTTCAATA
CCAATTTTGATGAGAATAAAGTGGATGTAACTCAATTCAAAGCTACCTTTTTCCCAAAAGGCTTTAAAGATAA
AACTGCTGGACTTACATTTCATGCTTTGAGTGGCAAAAATATGTGCAACTACCAGCTGGTCTGCGACAGTGAT
GAAAACCTGAAGAATAAAGAATCTGTGGTCCAGTGTCTGCATATCTTGTCCTCCTTAAGGCTCATCATGCAAG
TGGCTCTGCCACAAGAGCATCTTTGCTGGATTATCTTCAATGGTACCATTTATATTTACACCATTTGCAGAAA
ACTGATGGTCATAGGTCAGTCTTCCAAGGCCTTAGAGTATCTCCTGTGGGCCAGCATGTGTATGGAGTCCTTG
GTCCCGCTCCTGTCACTCAGGTACTTGACATGGCGCGCTACTCTCTACACAGCTGTTTGCCAGTGCTGCTATG
ACTGCCATGCCGGGATTCACGGAGAGGCATTTGCTCGGCGTGCTTTGGCTAAAATTGATGAGTTAAGGCAACT
GGAATTAATGAGTTCCTCAAAATCCCAGGAAGAATCGCGAAGATATTTTCGAGAGGCCACAATGAAGATGGCA
GTGATGATCTTCAAAAGAGGAGTCTTTGAATCTAGAAGAAAAAACAAAGCTGTCTTTAGACCTAAGATAAGAA
TTAACCTAAGGGAAGTACAAACTTTATCATGGCCACGAACTGTCACAGAGCGGCTACTGGATGAGATGTTTGA
TAGCACTGCATCCCAGTTTCTGGCTGTCTTGGAAGCTCTTTCTGATTCAAATAGGCGAATACTGCAAACTGGA
CCCATTGTTACAGATGAAGTGGAGATTCATGATGTTGTCTCAGAATTGTTTATGGCAGGAAAAGAACTTTTGA
TAATGTCAAATATTGGTGCAGATGGAATGCTTGATTTTCCAAAAACATCTCTTCTGGAACTTATGATAGGAAG
AAAAGATGTTATTTCTGTGGATGCTGCTGTGAAATTTATAAAATTAGCTTTTACCTATGAGGAGTGGAGTTTA
TTTGAATCTTCTGCTGTACATCTTATTTATTTTCTCCAGAGGCAGGATGATCCAGAGTCAAAGAAAGCAGAGA
AGGATTTAACTCTTCTGATTGCAATGGAACCACTAATCAACGTGAAGAGAAACAAAGGTTTGATCTTTCCTTT
GGAAAACTATAAAGAAGGACAGTCAACTCAAATTTATTTAAAAAAAATTGCTGTTCATGATACTTGTTTGAAG
ACTTGTGGATATTCAGAGGATATTTTCCATTTGGCAGCAACCTTGTATGTCTGTGTCTGCACTGCTCCCCAGG
ATGTTCAACCTGATAAAGAAATTGTTGTGGACACGATAATGTTCCTATGGCAGAAATGCAAATTAGGAATTCA
GCGGCTCAATATATCCAGAAATGACTATGCAAAATTCACCCAGAAAATCAGTACTAACAAATGGATTTATCTT
CTGTGGCAGATAAATGAAGTAATTCACTGCTATAAAATGGAAGACATTGACATTGTGGTAGTGGCAGAAGTCA
CATTACGGTTAAGTGAAATATTGGAATCTTTAGGAAGCCCAGGAAGAAAATTTAAACAATCTCTAGATGTACC
TTTAAGAGAAGGGACTAACAAATTCCCTGGAGCTCCAAAAGGGATCACAGAAATTCTTCCAATATTACAGAAA
AATCCTGTGGAACAGTTACTTTTTGCTTATAAACTTCTTGACAGAGCAATCGGTGGAATAAATTTGAATTGCA
TGTTAACCTCTTTGCCAAATGGATCATCAGTAATTGACCACTGCTATGCCAAGCGTACCCACCATATAGATGG
AGATACTTACAAACCACTTGCCTCAAATAGTTTCATGATGGATTTGCATCTTGAACTAATTCAAGCTCAGCAT
CGAATAGCTGTTGTGCTTCTGGACAAATTGCAAGTTTTGCAGACTCCAACTGTTAGCAAAGATATTTCTACCA
AGGGTCCGGAAAAGTTAAAACAATCTGGAAGCACTGATTGTTTTACAGAATTAAATATAATGAATAAAATAAA
GAAGAATACATTATCCAAAGCAATTTACTTAATGCAGAAAGCTCTTTTAATATTCGAGAAAGATGCAACTTCT
ACATCTTCATGGGAACTTTTGATGGAAGCATATTCCTTAATTCAGAGGATTGAAGCTGAACAAAATGCCCTAT
ATTCCTATCAGAAATATTTGGAAAGTTCAAAAAGAAAGAAAAGCAGAGTCCCCCCTCCACCTATCCTGCTGTC
TCGAACTCATTGTTCTGTGACACTCAAACCTGCTCCATTTACTTCAGAGGTTAAGGTCTCCTGGTACTGCATT
TTGGGTTGCAAAGCAGAAGGAAGTTATGGAAAAGTACGGCTAAACAATAATCATCTCCCAAATTCAGGAGAAG
CGATACCAGCTGACGGTAAAAGTGTTTTTGAAGTGAAAGGTTTAGAAACCAATGAAAAGTATGTATTTGCAGT
TGCTGCCTATTCTAACAACGGAAAGCTTGTCGGTGGTGCTATTGGGGAGACAACTAAACCAATTCTGGTTTAT
CCCCCTCTTTCTACTATTACTGCTCGGATGTTCCTGACACAGGTTGCCTATCAAGTTGGTAACTATGAATTGG
CTAAGAAAGTTTTCTCACCAGTTTGGGATTATTTTGTTGCTTCGCCACTTCAGGATGAACAATCTGTTATTTG
TTTAAGCAATATAATTACTATTACACAAAGAAGATTACATTCAGATATTTTGGCAGAGACTTCTTCAATCCTC
TTGTACCTTTTTCTTAGAAATATTTTTGTAACAAGTGACATCAAAATTAAAGAAGAAAATCTTTTCTGTGATA
ATATTAAAGGCAATGAGATTTTCCCATCTCAACAAATTGCCAGACTGATTGAATGTGAGAGAGTATTAGTGGC
ATTGGAACTTAGCAACTTCCTAAATGATTCCAGCTATGCCCTTCAAGCTGTGACTCAATGTTATGGACTTCTT
GCTCCCATAATTTATCACAATATTGTTTTGGTACCTGTTGTACAGATCCTGATAAAGTGTATAGTGGTTTTGC
AAGGACTACCAAGTATTGTCTGCTCGAAGAAACATACTGCGAGCTTTGAAAGTATACAACACATGATAGCTTG
TTGTATTTTCTACATAACAAAGATTCTTCGTTCATGGAGGGAATATGACCTGGCAGTAATGATTATAAATTAT
GGGAAAAAAATGTTGGACATCACACCAGGATGCAAGTCTCTGTTTGATGGTAGTAATGAACAAGAAGAAATGC
CAGAGGAGGATTCTTCTAAGAAGTCTTTAAAGACTAAGAAGCCACAGCAGATACTACTGCCTGAAAAGATAAA
TGAACAGCTGGCCTTGTTGGAGACACACCTACTCAAACTGACAAAGCAATATGTTACATCTGAACTCTCAGGA
GGAGAGGACCCTATATTTCTTTATCCTGTAGTTTTGAATTGGTCGGTCAAAGGTGCCGTGAAAGAAGTTATGA
AATTTAAGCAAAAACCAAGATTTCTGGAATTCTTTACACAAGTTATGCTAAAATGCATGAATGAGGAAAAATT
TCATCTTATGGTAGAGGTAACAACTCCTGTCCATGACTTCTTGAAAAGGAGAAATGAGTCTCTACTTGGACTA
ATAAAAGTGAAATATAAGGATAGTGCTTTGAATAAAAAAGCAAACAAATCTTTAAAGTTCAAAGCTGCAGTAA
TGGAAATTGGAAGAAGTGCAGAAATGCAACAAAGAATAAGAAGTAAAAAAAAGGAAACTCTAAGAGATTTCAT
TTTTAAAAATCCGGCTATTTCTGAAATGGTGGCACATGAAAGAAATAGGAGAACCAGTGTTAGGAAAGCAGCA
CAACGATACCTGATGGATTACTTGAATCCTCTAATATTAAGTTATGTTAAAAGAAAGAGGTTCCATCGTCTAT
CACTTGAAGAGATGCCCTGGAGAGCTCAGATGAACCTGTATCTAGCAGGTGCACACTTTAACCTGGTTTTACA
AAAGCTATGGGAGTGTACGAAGATGAAATTTGGCACATCACATATGATGGTCAGTTTCCGATCATGTGATCCT
AACATGTTTTCACTGTATAATTCAGGAACAGTATTACCAACAAGAAAATTGACTGTAGAAAATTATAAAGCAA
TGCTTGATTTCCTTCTTACAGCCAAAAAAAGAAAGGCCAACTTACCATCAGATGCTGAAGAATTTTCTACATT
TATTAATTCCATAATGAGTGATGAAAATATGTCCAAGACACAAACAGTTTATGACTCAGACTCTCAATCAGGT
TCTAGTGCTAAAGAAAAGGACCGAGGAGCAAATTTGTGTGTAATGGATCATTTTATGAAAATCTTTTTATACT
GCAGGAGAGCAATGGTTCTTGCTCATCGTGGTGGCTATTGGACTCTGCTTCAGAACTGCTGTCGGGCCTTATG
GAACTTTACTCAGGAACTACAAATACTTCTTAAACAGGCAGTGGATCTTGATAAAACATTTCCTATTAGCCAA
GATGGTTTCCTCTGCACCTCTGTTTTACCATTCTATTTGGGAGCAGAATTACTTATTGACATGTTAATACAAC
TACAAAATACCAGTTCTATTAAGCCTATTGAAGACAAAGGAGAATTCAGTGTTCCAAGCTGTTATGGGAATAT
TAAAAATGACAACGGTGGTTCTAGTCTTACCTTTGAGCATCCTTTGGATGATGTAAATGTGGTTGATTTGAAA
TGGATCCACGACTTTGTATTAAAATCTCTGGAAGTTTTATATCAAGTGGAAAAATGGGAAACACTAGTATCTC
TTGCCATTCAGTTCAATACAGTTTCACATGAGAGGTATACAGAACAAGTGACACCACTTCTGGTGTATGCACA
GCGCCAGCTTCTGCTGAGAATACAGAAGTTCAAGGGCCCAGATATTACCCAACAACCTTGTGCAAGGTATGAG
GCTGAATATGGAGAGAAGATAACTTGCCGAAATTTCATTGGGAAGCAGCTTAAGATTAATTCTTCAACCATTG
AAGCAACAAGCAACTGCACAGATTTGCTAAAAATGCTTATCTCTTCAGAATACAGCCGAGCCAAAGCGCTTGT
CTGCGTGCCCGTGGACGTGACAGACACCTTGAGGTGTTTTAGAGAGACACTGGAAAAATCCAAATACCATAAC
AGATCAATCCGACACAGCAGAAAGTTGCTTTCATTATTTCTTGCACAGACACAAGATGTTCTCCAAGCCAGCA
ATCAAAGAAGTCTTAAAGTTCAGGCGTTGCATTCACTTGGAAGTCTTCTCATCTTCGCAGAAAAGAAAAGGGC
TGCTTTTAAGTGTTGGTGTCAAGCTCTTGATGACATATTCAGAAAACCAGACGTGCTACACACGTGGAAAGAA
TTTGGCCCCTCACTCACCAATGTCACCAACAGTCATTCACCTCCGGGTTTCAAAGACTACAGTGAGGAGTTTC
TGTCAAGAGTTGGCATCTGGGGGTGTTTGCAAGGAGCAGTCATATCAGCAAAGATAGCACAATTTATTAAGTC
ATTGAATGTTGAAAAGAAAACTGACTGTTGCATTTTGTCTGCGTTACTCTTTCAGGGTTTGCTTAGAACAACA
CTTCCACATCCCAAAGCTGAACGTTGCTATGCTCAATATGAAATCACTCAGCTTCTCCCAGGCATTGAACTCT
TCTCAGATAGATACAGGGCTGACATTTGCTCTGTAATTGCAAGTCTGTATTACATTATACGTGAACTGCACTT
TGTTAGGCAAAACCTAATAGTTCTGCCTCTCCTTGCATTGTATCAATATTTTGTTTCTGGAATTTGTCAAGAC
ATAACAAGAAATCTAGAAGCAAGAATCCTCAAGATAGAAGTCCTTATAGATTTGAGATTCTTTTCTGAAGCCT
TTTATGAGATATCCCAAATTTTCTATGGAAAAAACATGCCTTGTCCAATACCTGCAGGCTATAAAGCCACTGG
AAAAATGAAGATCTTTCAATCATTTGACTCAGGAAAACTTCTTACCAGTAAAGAAAATATACAGGCAATTGAT
GAATTAAGAAATAAAGGCTTGCCTGCAGTTCTGGTTACAATTGGCCAACCACATCTCTTAAATAAGTTTAATT
TTGTTAAAGCATACTTTTTCCTAAGTGTGGCTGCGACAATAAATTGTGTCCCAGAAAATAAATTTAAGACAGT
AATTACCAACAAGAGCAAACCAAACCTACCAAACTTGGAAGAGATATATTCAAAGGATGATGGAAGTTCATTT
TATAATCTTACAAAACTTAAAGATGAGATCACTCTTAGCATGCTAAAGTCGATGTTACTGATGGAAGCTGAGG
ACAGGCTAAACTTCCTTCTGTCCGAGGTGGAACAGAAGACCCTGTCTCAGTGCTCCGCTGGCGAGCTGGAGAT
TGTGGTGGAGGCCCGGCTTCAGCTGGCTGCAGTTGCTCTGCAGAGGCACCGGGCGGCATACAGTGCTGCAATA
GTATTTTCTACACTTACACTTCTCCAGGATTCAAAACTTTTTGAAAAGAAGGTAGTACAGGATGACACAGAGA
ATCCTGTCTCTCCAGGAACTTCTGTCACTGAAAATAAAGATGACAGTGAGTTTTTAGATCCTATTTCCCTAAA
TGCCCGAGAATATTTCAACATTCATCTGTGGTTGAGGTGCCGCTTAGCATTGGTGACTGCATTTGTTGCACAG
ATTCATGGCATTGGAATTGTGAAAGAGGATGATATGACAGATTGCCTGAGCCTCATCAATGAAGTGTGTATGG
AGGCAAAAAGCGCAGGGGACACGGAACTGCAGGCTGAATTCTTGACGCAAGCTGTAATTCTTGGCCTACAAGA
AAAGCATTTAAAGGCAGACATCATGACAAACCTTCAGGATATAATACATTTGCTGGAAGGAAATGAATTTATT
TCTCCTCAATCACGGCTAACCCTGGCAAGAAGCCTAGTTTTGCTGGATGACTTAACCAAAGCTGAGAAATTCA
AGGAATCTCCCTCTTCAAAAACAGGAAAATTAAATTTGTTAACTCGGGCTCATAGCATTCTAACTGAACAGAT
GCTAGCTTTTGGAGAAACAATTGAATTTCGTTCATCAAACACTAAATATGCAAATCCATTACAGCCTTTGAAA
AATATCTATCTTCCCCATGTCATGTTATTGGCCAAAATAAAAATGAGAATTGGACATACAGTGGCCAAGCAAG
TATATTACAAGAATAAAAGGAAGGACCCCTCGAAGTGGTTACCTGCTCTTCATCTGTTTGATGTGGCACTGAA
GCTCTGTAGAACAACAGCAGTGGAGGAACATGAGGTGGAAGCTGAAATCCTTTTTCAGAAAGGCAAAATAGAA
CGTCAAATACTAATGGAAGAGAAATCTCCAAGTTTTCAACTTGAGAGTTTATATGAAGCTATACAACTAAGCC
TGAAAAATGATCAAAACTCAGGATTGATAAGAGACTCCTACCTAGAAATGGCTCTATTGTATTTTCATCTGAA
GAAGCCAAAGATAAAAATTTCAGGATCACCATTAACACTTAAGCCTCCTCTCAGAAGAAGTAGTTCTGTTAAA
GAAACATCAGCAAATAAATTTGAAATGTACAGTTCATTAGCCTGGATTGCAATAAGAGCTGCTGCACAGGTCA
GTGAAGCTGTGCTGGCAATTAACTTACTTATTGGAAAGAAGAATACTAGAATGCATAAAGTTAACCAAGTGGC
ATTACCAAATATCCCAGAATTTGCTGCTCTGGATCTTTTGTCTTCGTATACAGATTATTTGCTTGACAACTAC
CAAGTCCTCTTCCAGACTTCCTGTACATTTTTGTACCAAAATGATGATGTGTGTGACAGCGCAGATGGTAGAA
AAAAGACTCAGACCAAAGTGGATATTACATGGATCCTTCTACTGCGCTACTATATTCACCTTCAGAGGATTAA
TAATCTGAGCAAACTGCTAGCATCTGCAACACCAGTATCTGGAATTTCTTTGCCAGATGATACACTTCTCACA
TCCCTTTACAACTCTGAGTTGATTTTGCGCCAGAAAGAAGTGCATTTTTTCCTTAAAAAATTCTTACAGCTGT
ATTCTTCTTCTTGTATTGATGAATTTCCAAAAGAACTTCTTTGTCAACTGGAAAATCCCCCTCTTTCAGAAAA
AGACTTACGTGAATCATCTGCCAAGTTGTATCGCGATAGTTCTGTACAATCCATTTTATCTTTTAAGCCTGTT
TCAGGCTCATCTTGTGTGGACATAACGCCAATAGAAATGGTAACGCAAGCTTCAAACAAAGAACTTTGCTTTC
AATGGTACATTCCTCCCCTGGATAGACCTCCCAAGGAGACAGAACCTATGGTTTTATTGTTGTATGCATATAA
TTTGAAGCCTCTGAAGATTTCAGATGTTAGACATTCCACTTATAACAGTACATGTGTTGGCTCTTTATGGATT
CCACTGAATAGGGTTATTGCAATTCATGAGAAATTATCTAATCTTGCTCAAATAGCTGAACTATCATTGCCAG
CAGCTCCTGAAATTACCTCAAATGAAAACATATATGAAGTAGAAGTGGAAGAGGAATCAGTTGATAATGAAAT
GGAAGACATGATTATTCAATGTTGCTCTGAAATAGCATCTCTGTTTTTGAATGATAAAGAGCCAACACCACTA
TCTGAGGTCCCATTTGATATCTCACTGCCGTCTATATTCAATCTTGAGAGACTTTTTGATCTGGCTAATGGTT
GCATTTTATCAGGAGGAAGCCTTTTCAACTGGATAGTGTCAATTATTCCCTGAATATCCTATACACGGGATGA
TAAAACTGAGAAATAAATCAAGAGTATGACAACAGACTGAGGAATTTTTCAGCAATATCTGTCAGCCTTTGGA
AATTTGGTGAGAAGAAGTAGTCATGATTCAATGTCTTGCTTGCACAGAAGCCTCTAGTTCAAACTTGGATCTT
GCCCCATCTGATTTCTTATGATATATACAAATACTCTAGATATTGCTTTGAGATTTCGCTGTTGATCAACTGG
CCTGCTGCAGCTTCATAGGTGTTTCAGCTCCCAGTAGGGCAGTTTAACTCCTATCTTCTCTGTGAGTTCCAGG
TCTGTATATCTCCTCAGCTCGCTCTCATTTGTCCAGCTTATCTATTTCTTTATCAAGTTGCTTTGGCTTCCCC
ACTCTCTCCTGCGTTTTGAAAAGAATTAATTCTCAAGTAGATTAAAACTACATTCTGGTAAAAATGA
(SEQ ID NO: 141)
MAAQGSPSSSPSDDSTTSGSLPELPPTSTATSRSPPESKGSSRSSLLQWTCPEDSLPLAVFYGPLDAKNPLLA
SCEKEIQELLGFMRKKKALATTEEEKHEFRRRCATSLFNIWTKYAPRLPADYYNEKLLKVGDSLCQMKEYKLA
LLQCYGRYLQQFNTNFDENKVDVTQFKATFFPKGFKDKTAGLTFHALSGKNMCNYQLVCDSDENLKNKESVVQ
CLHILSSLRLIMQVALPQEHLCWIIFNGTIYIYTICRKLMVIGQSSKALEYLLWASMCMESLVPLLSLRYLTW
RATLYTAVCQCCYDCHAGIHGEAFARRALAKIDELRQLELMSSSKSQEESRRYFREATMKMAVMIFKRGVFES
RRKNKAVFRPKIRINLREVQTLSWPRTVTERLLDEMFDSTASQFLAVLEALSDSNRRILQTGPIVTDEVEIHD
VVSELFMAGKELLIMSNIGADGMLDFPKTSLLELMIGRKDVISVDAAVKFIKLAFTYEEWSLFESSAVHLIYF
LQRQDDPESKKAEKDLTLLIAMEPLINVKRNKGLIFPLENYKEGQSTQIYLKKIAVHDTCLKTCGYSEDIFHL
AATLYVCVCTAPQDVQPDKEIVVDTIMFLWQKCKLGIQRLNISRNDYAKFTQKISTNKWIYLLWQINEVIHCY
KMEDIDIVVVAEVTLRLSEILESLGSPGRKFKQSLDVPLREGTNKFPGAPKGITEILPILQKNPVEQLLFAYK
LLDRAIGGINLNCMLTSLPNGSSVIDHCYAKRTHHIDGDTYKPLASNSFMMDLHLELIQAQHRIAVVLLDKLQ
VLQTPTVSKDISTKGPEKLKQSGSTDCFTELNIMNKIKKNTLSKAIYLMQKALLIFEKDATSTSSWELLMEAY
SLIQRIEAEQNALYSYQKYLESSKRKKSRVPPPPILLSRTHCSVTLKPAPFTSEVKVSWYCILGCKAEGSYGK
VRLNNNHLPNSGEAIPADGKSVFEVKGLETNEKYVFAVAAYSNNGKLVGGAIGETTKPILVYPPLSTITARMF
LTQVAYQVGNYELAKKVFSPVWDYFVASPLQDEQSVICLSNIITITQRRLHSDILAETSSILLYLFLRNIFVT
SDIKIKEENLFCDNIKGNEIFPSQQIARLIECERVLVALELSNFLNDSSYALQAVTQCYGLLAPIIYHNIVLV
PVVQILIKCIVVLQGLPSIVCSKKHTASFESIQHMIACCIFYITKILRSWREYDLAVMIINYGKKMLDITPGC
KSLFDGSNEQEEMPEEDSSKKSLKTKKPQQILLPEKINEQLALLETHLLKLTKQYVTSELSGGEDPIFLYPVV
LNWSVKGAVKEVMKFKQKPRFLEFFTQVMLKCMNEEKFHLMVEVTTPVHDFLKRRNESLLGLIKVKYKDSALN
KKANKSLKFKAAVMEIGRSAEMQQRIRSKKKETLRDFIFKNPAISEMVAHERNRRTSVRKAAQRYLMDYLNPL
ILSYVKRKRFHRLSLEEMPWRAQMNLYLAGAHFNLVLQKLWECTKMKFGTSHMMVSFRSCDPNMFSLYNSGTV
LPTRKLTVENYKAMLDFLLTAKKRKANLPSDAEEFSTFINSIMSDENMSKTQTVYDSDSQSGSSAKEKDRGAN
LCVMDHFMKIFLYCRRAMVLAHRGGYWTLLQNCCRALWNFTQELQILLKQAVDLDKTFPISQDGFLCTSVLPF
YLGAELLIDMLIQLQNTSSIKPIEDKGEFSVPSCYGNIKNDNGGSSLTFEHPLDDVNVVDLKWIHDFVLKSLE
VLYQVEKWETLVSLAIQFNTVSHERYTEQVTPLLVYAQRQLLLRIQKFKGPDITQQPCARYEAEYGEKITCRN
FIGKQLKINSSTIEATSNCTDLLKMLISSEYSRAKALVCVPVDVTDTLRCFRETLEKSKYHNRSIRHSRKLLS
LFLAQTQDVLQASNQRSLKVQALHSLGSLLIFAEKKRAAFKCWCQALDDIFRKPDVLHTWKEFGPSLTNVTNS
HSPPGFKDYSEEFLSRVGIWGCLQGAVISAKIAQFIKSLNVEKKTDCCILSALLFQGLLRTTLPHPKAERCYA
QYEITQLLPGIELFSDRYRADICSVIASLYYIIRELHFVRQNLIVLPLLALYQYFVSGICQDITRNLEARILK
IEVLIDLRFFSEAFYEISQIFYGKNMPCPIPAGYKATGKMKIFQSFDSGKLLTSKENIQAIDELRNKGLPAVL
VTIGQPHLLNKFNFVKAYFFLSVAATINCVPENKFKTVITNKSKPNLPNLEEIYSKDDGSSFYNLTKLKDEIT
LSMLKSMLLMEAEDRLNFLLSEVEQKTLSQCSAGELEIVVEARLQLAAVALQRHRAAYSAAIVFSTLTLLQDS
KLFEKKVVQDDTENPVSPGTSVTENKDDSEFLDPISLNAREYFNIHLWLRCRLALVTAFVAQIHGIGIVKEDD
MTDCLSLINEVCMEAKSAGDTELQAEFLTQAVILGLQEKHLKADIMTNLQDIIHLLEGNEFISPQSRLTLARS
LVLLDDLTKAEKFKESPSSKTGKLNLLTRAHSILTEQMLAFGETIEFRSSNTKYANPLQPLKNIYLPHVMLLA
KIKMRIGHTVAKQVYYKNKRKDPSKWLPALHLFDVALKLCRTTAVEEHEVEAEILFQKGKIERQILMEEKSPS
FQLESLYEAIQLSLKNDQNSGLIRDSYLEMALLYFHLKKPKIKISGSPLTLKPPLRRSSSVKETSANKFEMYS
SLAWIAIRAAAQVSEAVLAINLLIGKKNTRMHKVNQVALPNIPEFAALDLLSSYTDYLLDNYQVLFQTSCTFL
YQNDDVCDSADGRKKTQTKVDITWILLLRYYIHLQRINNLSKLLASATPVSGISLPDDTLLTSLYNSELILRQ
KEVHFFLKKFLQLYSSSCIDEFPKELLCQLENPPLSEKDLRESSAKLYRDSSVQSILSFKPVSGSSCVDITPI
EMVTQASNKELCFQWYIPPLDRPPKETEPMVLLLYAYNLKPLKISDVRHSTYNSTCVGSLWIPLNRVIAIHEK
LSNLAQIAELSLPAAPEITSNENIYEVEVEEESVDNEMEDMIIQCCSEIASLFLNDKEPTPLSEVPFDISLPS
IFNLERLFDLANGCILSGGSLFNWIVSIIP*

An amino acid sequence for the protein encoded by the CISH gene that is a regulator of T cell exhaustion/dysfunction resistance is available from the UniPROT database at accession no. Q9NSE2, cDNA NM_013324, with gene location at Chr 3: 50606489-50611774.

(SEQ ID NO: 142)
TCCCTGTCCTGCGCCCGCGCGCCCCGGGAGCCTACCCAGCACGCGCTCCG
CGCCCACTGGTTCCCTCCAGCCGCCGCCGTCCAGCCGAGTCCCCACTCCG
GAGTCGCCGCTGCCGCGGGGACATGGTCCTCTGCGTTCAGGGGTGAGCAC
CCCCTTGTAAGCTCAGGGCTACTGTTGGGTGTCAGGGAACAAAGTTTTAG
ACTGCTGCGCTCCAAAGCGGGCACACACATGTACCTAGAACACACCAGCC
ACTGTCCCCACCATGATGATGACACAGCCATGGACACACCCCTGCCCAGA
CCTCGTCCTTTGCTGGCTGTGGAGCGGACTGGGCAGCGGCCCCTGTGGGC
CCCGTCCCTGGAACTGCCCAAGCCAGTCATGCAGCCCTTGCCTGCTGGGG
CCTTCCTCGAGGAGGTGGCAGAGGGTACCCCAGCCCAGACAGAGAGTGAG
CCAAAGGTGCTGGACCCAGAGGAGGATCTGCTGTGCATAGCCAAGACCTT
CTCCTACCTTCGGGAATCTGGCTGGTATTGGGGTTCCATTACGGCCAGCG
AGGCCCGACAACACCTGCAGAAGATGCCAGAAGGCACGTTCTTAGTACGT
GACAGCACGCACCCCAGCTACCTGTTCACGCTGTCAGTGAAAACCACTCG
TGGCCCCACCAATGTACGCATTGAGTATGCCGACTCCAGCTTCCGTCTGG
ACTCCAACTGCTTGTCCAGGCCACGCATCCTGGCCTTTCCGGATGTGGTC
AGCCTTGTGCAGCACTATGTGGCCTCCTGCACTGCTGATACCCGAAGCGA
CAGCCCCGATCCTGCTCCCACCCCGGCCCTGCCTATGCCTAAGGAGGATG
CGCCTAGTGACCCAGCACTGCCTGCTCCTCCACCAGCCACTGCTGTACAC
CTAAAACTGGTGCAGCCCTTTGTACGCAGAAGCAGTGCCCGCAGCCTGCA
ACACCTGTGCCGCCTTGTCATCAACCGTCTGGTGGCCGACGTGGACTGCC
TGCCACTGCCCCGGCGCATGGCCGACTACCTCCGACAGTACCCCTTCCAG
CTCTGACTGTACGGGGCAATCTGCCCACCCTCACCCAGTCGCACCCTGGA
GGGGACATCAGCCCCAGCTGGACTTGGGCCCCCACTGTCCCTCCTCCAGG
CATCCTGGTGCCTGCATACCTCTGGCAGCTGGCCCAGGAAGAGCCAGCAA
GAGCAAGGCATGGGAGAGGGGAGGTGTCACACAACTTGGAGGTAAATGCC
CCCAGGCCGCATGTGGCTTCATTATACTGAGCCATGTGTCAGAGGATGGG
GAGACAGGCAGGACCTTGTCTCACCTGTGGGCTGGGCCCAGACCTCCACT
CGCTTGCCTGCCCTGGCCACCTGAACTGTATGGGCACTCTCAGCCCTGGT
TTTTCAATCCCCAGGGTCGGGTAGGACCCCTACTGGCAGCCAGCCTCTGT
TTCTGGGAGGATGACATGCAGAGGAACTGAGATCGACAGTGACTAGTGAC
CCCTTGTTGAGGGGTAAGCCAGGCTAGGGGACTGCACAATTATACACTAT
TTATTTATTTATTCTCCTTGGGGTTGGTGTCAGGGGCGAGCCAACCCCAC
CTCTATGCCCTGAGCCCTGGTAGTCCAGAGACCCCAACTCTGCCCTGGCT
TCTCTGGTTCTTCCCTGTGGAAAGCCCATCCTGAGACATCTTGCTGGAAC
CAAGGCAATCCTGGATGTCCTGGTACTGACCCACCCGTCTGTGAATGTGT
CCACTCTCTTCTGCCCCCAGCCATATTTGGGGAGGATGGACAACTACAAT
AGGTAAGAAAATGCAGCCGGAGCCTCAGTCCCCAGCAGAGCCTGTGTCTC
ACCCCCTCACAGGACAGAGCTGTATCTGCATAGAGCTGGTCTCACTGTGG
CGCAGGCCCCGGGGGGAGTGCCTGTGCTGTCAGGAAGAGGGGGTGCTGGT
TTGAGGGCCGCCACTGCAGTTCTGCTAGGTCTGCTTCCTGCCCAGGAAGG
TGCCTGCACATGAGAGGAGAGAAATACACGTCTGATAAGACTTCATGAAA
TAATAATTATAGCAAAGAACAGTTTGGTGGTCTTTTCTCTTCCACTGATT
TTTCTGTAATGACATTATACCTTTATTACCTCTTTATTTTATTACCTCTA
TAATAAAATGATACCTT
(SEQ ID NO: 143)
MYLEHTSHCPHHDDDTAMDTPLPRPRPLLAVERTGQRPLWAPSLELPKPV
MQPLPAGAFLEEVAEGTPAQTESEPKVLDPEEDLLCIAKTFSYLRESGWY
WGSITASEARQHLQKMPEGTFLVRDSTHPSYLFTLSVKTTRGPTNVRIEY
ADSSFRLDSNCLSRPRILAFPDVVSLVQHYVASCTADTRSDSPDPAPTPA
LPMPKEDAPSDPALPAPPPATAVHLKLVQPFVRRSSARSLQHLCRLVINR
LVADVDCLPLPRRMADYLRQYPFQL*

An amino acid sequence for the protein encoded by the COPS7B gene that is a regulator of T cell exhaustion/dysfunction resistance is available from the UniPROT database at accession no. Q9H9Q2, cDNA NM_001282949, with gene location at Chr 2: 231781671-231809254.

(SEQ ID NO: 144)
ATCCCCTGCACTCGGTTCCTCTGTAACTTAGACTCCAGTGTGCCGCGCAG
TGTGCGCAAATTCACAAACCCGCCCGCTGAGTTGGTCGTTTCGGAATCCC
GAGACTTGAAACCCAACGGGAAGACCCTTCTCACCCTCAAAAGAAGATGA
CAGCCCGCAGAGAGTTAACTTCTGTTTACATGGAACTCAGACATCTGCAA
ATCATGCTTGCGGAAGGAGCTAATGCTGCTTATTTGCAGTTGTTGAACCT
GTTTGCCTATGGGACATACCCAGATTACATAGCCAACAAGGAGAGCCTGC
CAGAACTGAGCACAGCTCAGCAGAACAAGCTGAAGCATCTTACCATCGTG
AGCTTGGCATCAAGAATGAAGTGTATCCCCTACTCCGTGTTGCTGAAAGA
CCTGGAGATGCGGAATCTCCGGGAACTAGAAGACCTTATCATTGAGGCTG
TCTACACTGACATCATCCAGGGCAAGCTGGACCAGCGAAACCAGCTGCTG
GAAGTGGATTTCTGCATTGGCCGTGACATCCGAAAGAAGGATATCAATAA
TATTGTCAAGACCCTGCATGAATGGTGTGATGGCTGTGAAGCAGTTCTAC
TGGGCATCGAGCAGCAAGTTCTGAGAGCCAACCAGTACAAAGAGAACCAC
AACCGAACTCAGCAGCAGGTAGAAGCAGAGGTTACCAACATCAAGAAGAC
ACTCAAAGCCACCGCATCCTCCTCGGCTCAGGAGATGGAGCAGCAGCTGG
CTGAACGGGAGTGTCCCCCTCACGCTGAGCAGAGGCAGCCCACCAAGAAG
ATGTCCAAAGTGAAAGGTCTGGTCTCCAGCCGCCACTAGGGCCGGCTGGG
GCAGCTGGCACTCACCAGGCCTGGGTCAGGTGGGGAGGGGACACCAAGGG
CCCATTTCCTCCCCTCTCTACCTGCAGTGAGTTCCAGACCTGCCCGTCCC
CTCACCAGCGCCTCCCCACCCTGTTGGTACTGTTCCAGAAAAACTGTTAC
TCCCCCTCA
(SEQ ID NO: 145)
MAGEQKPSSNLLEQFILLAKGTSGSALTALISQVLEAPGVYVFGELLELA
NVQELAEGANAAYLQLLNLFAYGTYPDYIANKESLPELSTAQQNKLKHLT
IVSLASRMKCIPYSVLLKDLEMRNLRELEDLIIEAVYTDIIQGKLDQRNQ
LLEVDFCIGRDIRKKDINNIVKTLHEWCDGCEAVLLGIEQQVLRANQYKE
NHNRTQQQVEAEVTNIKKTLKATASSSAQEMEQQLAERECPPHAEQRQPT
KKMSKVKGLVSSRH*

An amino acid sequence for the protein encoded by the CPSF4L gene that is a regulator of T cell exhaustion/dysfunction resistance is available from the UniPROT database at accession no. A6NMK7, cDNA NM_001129885, with gene location at Chr 17: 73248449-73262352.

(SEQ ID NO: 146)
GCTATATTGGGCCCACCTGCCTGGGTTCCAGCAGCTTCCGCAGCCCCAGC
AGGAGACGGAAGATGCAAGAGGTCATTGCGGGGCTAGAGCGGTTCACCTT
TGCCTTCGAGAAGGATGTCGAGATGCAGAAGGGCACTGGGCTCCTGCCTT
TCCAGGGCATGGACAAGTCGGCCTCAGCTGTGTGCAACTTCTTCACTAAA
GGGCTCTGTGAGAAAGGGAAACTCTGCCCCTTCCGACATGACCGAGGGGA
GAAGATGGTGGTATGCAAGCACTGGCTCCGGGGGCTCTGCAAGAAGGGTG
ATCACTGCAAGTTCCTGCACCAGTATGACCTCACCAGGATGCCTGAGTGC
TACTTCTACTCCAAGTTTGGTGACTGCAGCAACAAGGAGTGTTCCTTCCT
CCATGTGAAGCCAGCTTTCAAGTCCCAGGACTGTCCTTGGTATGACCAAG
GTTTCTGCAAGGACGGTCCTCTGTGTAAATACCGCCATGTCCCCAGAATA
ATGTGTCTCAACTATTTAGTTGGCTTCTGCCCCGAGGGACCCAAGTGCCA
ATTTGCTCAGAAGATTCGGGAATTCAAGCTGCTGCCTGGAAGCAAGATCT
AAGCGTTGCTTCTTACCTAGCGGGGCACTCCAATGCCACAGACAGGG
(SEQ ID NO: 147)
MQEVIAGLERFTFAFEKDVEMQKGTGLLPFQGMDKSASAVCNFFTKGLCE
KGKLCPFRHDRGEKMVVCKHWLRGLCKKGDHCKFLHQYDLTRMPECYFYS
KFGDCSNKECSFLHVKPAFKSQDCPWYDQGFCKDGPLCKYRHVPRIMCLN
YLVGFCPEGPKCQFAQKIREFKLLPGSKI*

An amino acid sequence for the protein encoded by the CUL5 gene that is a regulator of 55 T cell exhaustion/dysfunction resistance is available from the UniPROT database at accession no. Q93034, cDNA NM_003478, with gene location at Chr 11: 108008898-108107761.

(SEQ ID NO: 148)
GCGCGTCCCCGCCCTCGCGTCACGTGACGTGGCCGCGGAACCTGAGCTGCGGGGCCTAAGCCGAGCTAAATTC
GTTGCAGGTGGCCGCGGCGGGTGCAACCACAAAGGCTAATCCGAAGGAGTCGGGGAGGCTCGTGGAGTCGATG
CTTCCTCTTCCAAGTCAGGTCGGCTCCCGTTACCTTCTCAGCATTCGCCGTTCCGGTCTTCCTGAGCGCGTGC
ATGAGGTCTTTCGCGTGGGGAAGCTCCGGTGACCATGTAGGGGAGAAGAGTGAGGAAGCTCCTGGTGCTTGGG
ACGAGGTCAGCGCTGTCGGCGCGCTGCTCCAGCGCCCACCACACCCTGGTGCGGGCCGACGGGCCCTGGGCCC
TGGTGGGAGCTCCGGCCTCCGGTCAAGGCCTGGCCGGGAGCGCCACGAATTCTCGCGTCGTCTCGCGAGAGTC
CAAGTTAAAGAACATGGCGACGTCTAATCTGTTAAAGAATAAAGGTTCTCTTCAGTTTGAAGACAAATGGGAT
TTTATGCGCCCGATTGTTTTGAAGCTTTTACGCCAGGAATCTGTTACAAAACAGCAGTGGTTTGATCTGTTTT
CGGATGTGCATGCAGTCTGTCTTTGGGATGATAAAGGCCCAGCAAAAATTCATCAGGCTTTAAAAGAAGATAT
TCTTGAGTTTATTAAGCAAGCACAGGCACGAGTACTGAGCCATCAAGATGATACGGCTTTGCTAAAAGCATAT
ATTGTTGAATGGCGAAAGTTCTTTACACAATGTGATATTTTACCAAAACCTTTTTGTCAACTAGAGATTACTT
TAATGGGTAAACAGGGCAGCAATAAAAAATCAAATGTGGAAGACAGTATTGTTCGAAAGCTTATGCTTGATAC
ATGGAATGAGTCAATCTTTTCAAACATAAAAAACAGACTCCAAGATAGTGCAATGAAGCTGGTACATGCTGAG
AGATTGGGAGAAGCTTTTGATTCTCAGCTGGTTATTGGAGTAAGAGAATCCTATGTTAACCTTTGTTCTAATC
CTGAGGATAAACTTCAAATTTATAGGGACAATTTTGAGAAGGCATACTTGGATTCAACAGAGAGATTTTATAG
AACACAAGCACCCTCGTATTTACAACAAAATGGTGTACAGAATTATATGAAATATGCAGATGCTAAATTAAAA
GAAGAAGAAAAACGAGCACTACGTTATTTAGAAACAAGACGAGAATGTAACTCCGTTGAAGCACTCATGGAAT
GCTGTGTAAATGCCCTGGTGACATCATTTAAAGAGACTATCTTAGCTGAGTGCCAAGGCATGATCAAGAGAAA
TGAAACTGAAAAATTACATTTAATGTTTTCATTGATGGACAAAGTTCCTAATGGTATAGAGCCAATGTTGAAA
GACTTGGAGGAACATATCATTAGTGCTGGCCTGGCAGATATGGTAGCAGCTGCTGAAACTATTACTACTGACT
CTGAGAAATACGTTGAGCAGTTACTTACACTATTTAATAGATTTAGTAAACTCGTCAAAGAAGCTTTTCAAGA
TGATCCACGATTTCTTACTGCAAGAGATAAGGCGTATAAAGCAGTTGTTAATGATGCTACCATATTTAAACTT
GAATTACCTTTGAAGCAGAAGGGGGTGGGATTAAAAACTCAGCCTGAATCAAAATGCCCTGAGCTGCTTGCCA
ATTACTGTGACATGTTGCTAAGAAAAACACCATTAAGCAAAAAACTAACCTCTGAAGAGATTGAAGCAAAGCT
TAAAGAAGTGCTCTTGGTACTTAAGTATGTACAGAACAAAGATGTTTTTATGAGGTATCATAAAGCTCATTTG
ACACGACGTCTTATATTAGACATCTCTGCCGATAGTGAAATTGAAGAAAACATGGTAGAGTGGCTAAGAGAAG
TTGGTATGCCAGCGGATTATGTAAACAAGCTTGCTAGAATGTTTCAGGACATAAAAGTATCTGAAGATTTGAA
CCAAGCTTTTAAGGAAATGCACAAAAATAATAAATTGGCATTACCAGCTGATTCAGTTAATATAAAAATTCTG
AATGCTGGCGCCTGGTCAAGAAGTTCTGAGAAAGTCTTTGTCTCACTTCCTACTGAACTGGAGGACTTGATAC
CGGAAGTAGAAGAATTCTACAAAAAAAATCATAGTGGTAGAAAATTACATTGGCATCATCTCATGTCAAATGG
AATTATAACATTTAAGAATGAAGTTGGTCAATATGATTTGGAGGTAACCACGTTTCAGCTCGCTGTATTGTTT
GCATGGAACCAAAGACCCAGAGAGAAAATCAGCTTTGAAAATCTTAAGCTTGCAACTGAACTCCCTGATGCTG
AACTTAGGAGGACTTTATGGTCTTTAGTAGCTTTCCCAAAACTCAAACGGCAAGTTTTGTTGTATGAACCTCA
AGTCAACTCACCCAAAGACTTTACAGAAGGTACCCTCTTCTCAGTGAACCAGGAGTTCAGTTTAATAAAAAAT
GCAAAGGTTCAGAAAAGGGGTAAAATCAACTTGATTGGACGTTTGCAGCTCACTACAGAAAGGATGAGAGAAG
AAGAGAATGAAGGAATAGTTCAACTACGAATACTAAGAACCCAGGAAGCTATCATACAAATAATGAAAATGAG
AAAGAAAATTAGTAATGCTCAGCTGCAGACTGAATTAGTAGAAATTTTGAAAAACATGTTCTTGCCACAAAAG
AAAATGATAAAAGAGCAAATAGAGTGGCTAATAGAGCACAAATACATCAGAAGAGATGAATCTGATATCAACA
CTTTCATATATATGGCATAATTTTGAATATCATGGACAATATTTAGAACCCAAATTTTGGAGTGCTTGGGCAG
AAAGTTGTAAAGTTTGTGCTGGAGAAAGGTTTATTTGGACTTTGATTACATAAATATTAAAATCTCTGCCTTA
CCTTACAAAAACAACTATATTTTGCCAATCACATTAGTTAGCATGATGGCATTCCCTTCATGTTGCACACTCT
TTGACAGCATGCTGTTTTGTGGAGAAACTTGCATTCATGAAGAGCCCATTATCAACTTTTAAAAGTGAATTTG
ATTTGTACCCACCAGGAGAAATACAGTTGGGAAGGGTGAGGGTGGGGTTCTTGTTGCTTTTTTCTCTTTTTTC
CTCTCTTAAAAAATGTACTTGCACAAGGAAGGATCTTCAGATATTCATGCACTGAAAAATGCTGTTATCTTTT
GTTTTTAAAAAATGCAATTAAAACTAGAAATAGCACTCTTGGTTTCTTTTGATGAAAAGAGTGAGCTGGTCAA
TGCAACATTGGGAAACGGCAGATGTAGGTGAGAAGTTAAGTATTTAATAGTATCAAATTGTTCAAACATTGCT
CAGTATAATAACTGGATTTTAATGGTGACCTAAAAATGGTATTAAATATTTTCAACTTTCAAATGTTGGTTTT
CTTTAAGCAGGAGACTGCTATTTTAGTTGTTGTTAAATGATGAGGAGTTTTTTTTTTTTTAAATATGGCTGGA
AAGCTTGTTTTGAAAAATGAAGCTTATATTAATTGTTGCTTCAATAGTTTAAAAAATTTATGGAGATAGGTAG
GTCATTATGATTGCAGAGCCAAAATAGCAAAACAAACATATTAAGTGTGTTTACTAAACATATTGTGTGTTTA
CTAACACACTTAATGTTTATATACCTAATATTTATTATGCCATATCTGAATTTATTAAAAACAACACAGAAAT
CTCCTGCTCTGATATAGTTATGTAACTGATCTCTTAATCTGTAGCATAGAAGTATTTTGGTATTTTAAGGTCT
CATGATTAGGGATTTGTAGCCTCAGGTTTGAATCAGTCACCTAGGGGTAAGAGAAAAACCACAACATAAGACC
TGAAAAACAGAAGCAAAATTGTTGCACTCTTAATACGAGCAGTAAACTTGCCAAATATATGTACATATATATT
TATATATTTTAAAAATAAACATTTTTTAAATCGTCAGAAGGCAACATTTACCATGTTCCTTTCAGTCTATCCA
AAGTAGCAACTTAACTAGTTGCTGGCAACTGAATACCCAGAGTAGTGGAAATTTTAGGACTTGAGGACGTGAA
CAACTTGAAAGAGAAAATTGATTCCCCTTGAGGAAAGAATGGCTCAACGTGGATTTTTATTCATTGTGGTGCA
CGTTTCAAATTTTCTTGCAAATTATTTTATATCTTATTTGATGTTAAGTAATATTTTTAAAGTATGGTTTTTG
AAGATAGTAGGAAATGGAGTACAGAAAGGCATACAGTATTATGTTACAGTGGAGTGCTTTCAAGAGCATTTCG
TAGGATCTGTCCCCATCCGAAGAGGCTTTGTGAACTGCCTGCTGAATGGAAGGAAAGCAACTTTAAAACATTT
AGAGTTTTGCTGAGTATAGCCAAATGCCTGTTCTGAAACTTATAGGAAATTGGAATTTTGTTTAAAATTTTAC
ACTTTAAGGTAGCAAATGTCAATTATTAGACCGAGCATATTGTGAAGAATTAAACTAAAATTAATACGTACAG
TTTTCTCTAATTAGGTGACTGTTCATAATGGGTATATTTGGAATTTAGAAGAAATAATTTTGTAGTAATTGTG
GCCCTTATGTTTAACAGATAATTCAGCATTGGCGTATTTGCTTGTCCCAATACAAGAATGCCAAAGGAGGAAA
CAGGAAAAATTGCCGTCCATTTTAAAAATTTAGGTTTGTTGTTTTAAACCATAATTGTTCTCAGAACTTTTTT
GGAACTTCTAATTTTGGGTCATTTAATTTAAGAATGAGAGTCATGATGTTTTGATTTATGAAGGTGAATTTAA
TGCAAAAGTGGGTAACACTACATTCATAGCAAAGTTTTTTATTAAATTTTATAAATTTTTAATAGCCAATTAT
GTCCTAATTGTATTTTGGGGAATGAACAGCAGCAAATACTGTAAATGTACAGTTTTCTTTTTTTTTTTTTTTT
GGTTATGTATACAAAAGTTTTAACTACTTTTTGGTGTTTATGAAAATCATTTAGTTGACAAGGTGCCTATGCC
ATTGTTAAATTTTCTTTAAGTAATTTAACAACAGATTTGCTAATTTAAAAAAAAATGAAAAAAAAAATCTTAC
CAGCAGTTTGTAAAGGTCTGGAATCACAGTTGGCATCCTTTATTCAGGTCTCTTCTCTCCAGCGTACACTTAA
AATTGGTGCCGAGCAGGGATATAACCTGCAGTTAAGTGAAAAGAAAATCCAGCCTCCCCCTCCAAAAAAAAAA
AAAAAATTTAATTTTTAAAAATTAGTGGTATGGCAATAAGACACTTCAGAGGCTATCTTAACCTCTGAATACC
CATCTTCTAGTTTAAAGACAGAGACATCCCATCTGGAAAATGTTAACTTGTGTTGCCATCTCGTTGCCGGAGT
AAGTAGACATAAGACAGAGTTTAAGAAGTAAAAATATAGAAAAATTTTGATGGTCACAATGAGATAAATATTA
GAATATTACTATTCCAATGATTAAATGAGGATCTTGAAATAAATTCTGAAGTCTTCCAATTTTTACATTTATT
GGAGGGGTCCCTGAGTTCTGTCAACTTTTTTATTTAAGTCTCTTGCTCTTATTTTGTGCATAAATGTTAAACC
TTCCAAAAATGAAATGTTAGCTTTCTTTCTTTTACTTTTTATTAAATTTAATAGAAAATATGACCTGAGTAGT
TAAAAAGTATTTTGCATTATTTGCAGTAAGATGTCTCTAGCACTGCTCAAAGGGCAAATTTTAAAACTTCAGT
CTGGGTGAAAGATTTGCTAGTTTTACAGAAAGATTTGCTATCTTAAACTCAAGCTGGTTTTTCTGTTCTCATG
TAAGTGACTGGGATGCTGTCTTATGAATTCTTCCAAGGTCATGTTTGTGAAATAAACATTACATGAGAGCTTT
CCTGTCATCTACACTATATGTTGTCTGGAGTGTTGAACAAATTTATTTTAGTTTCTAAGTTGTAATCTATCCT
CATATGGTCTATACGATTTTGAATGTGTGCCACTACATACTGAGATGATAATGCTGTACAATTTTAAGTGGTA
GCAGTTTCTGTATGCAGTAGGCTGAAATATTTTGATGAACTGCTTAATTTTTGGATTTTATTTTTTAAGTTGT
ATAATTTATTTTCTTGCAAAATAAAAATGTAATATAAAA
(SEQ ID NO: 149)
MATSNLLKNKGSLQFEDKWDFMRPIVLKLLRQESVTKQQWFDLFSDVHAVCLWDDKGPAKIHQALKEDILEFI
KQAQARVLSHQDDTALLKAYIVEWRKFFTQCDILPKPFCQLEITLMGKQGSNKKSNVEDSIVRKLMLDTWNES
IFSNIKNRLQDSAMKLVHAERLGEAFDSQLVIGVRESYVNLCSNPEDKLQIYRDNFEKAYLDSTERFYRTQAP
SYLQQNGVQNYMKYADAKLKEEEKRALRYLETRRECNSVEALMECCVNALVTSFKETILAECQGMIKRNETEK
LHLMFSLMDKVPNGIEPMLKDLEEHIISAGLADMVAAAETITTDSEKYVEQLLTLFNRFSKLVKEAFQDDPRF
LTARDKAYKAVVNDATIFKLELPLKQKGVGLKTQPESKCPELLANYCDMLLRKTPLSKKLTSEEIEAKLKEVL
LVLKYVQNKDVFMRYHKAHLTRRLILDISADSEIEENMVEWLREVGMPADYVNKLARMFQDIKVSEDLNQAFK
EMHKNNKLALPADSVNIKILNAGAWSRSSEKVFVSLPTELEDLIPEVEEFYKKNHSGRKLHWHHLMSNGIITF
KNEVGQYDLEVTTFQLAVLFAWNQRPREKISFENLKLATELPDAELRRTLWSLVAFPKLKRQVLLYEPQVNSP
KDFTEGTLFSVNQEFSLIKNAKVQKRGKINLIGRLQLTTERMREEENEGIVQLRILRTQEAIIQIMKMRKKIS
NAQLQTELVEILKNMFLPQKKMIKEQIEWLIEHKYIRRDESDINTFIYMA*

An amino acid sequence for the protein encoded by the CUTA gene that is a regulator of T cell exhaustion/dysfunction resistance is available from the UniPROT database at accession no. O60888, cDNA NM_001014433, with gene location at Chr 6: 33416442-33418317.

(SEQ ID NO: 150)
CAGCCAGCAAGTGGCAGTTTCGGGTTCACACGGAAACGGGCGTGCCATTT
CCGCGCACGTCTGCAGATGCGGTAGTCGATTGGTCAAGTCTCCCATGGCT
CCTCCTTCATCAGGAGGTGGGCAAACCGCGCCATGATAGGGTCGGGATTG
GCTGGCTCTGGAGGCGCAGGTGGTCCTTCTTCTACTGTCACATGGTGCGC
GCTGTTTTCTAATCACGTGGCTGCCACCCAGGCCTCTCTGCTCCTGTCTT
TTGTTTGGATGCCGGCGCTGCTGCCTGTGGCCTCCCGCCTTTTGTTGCTA
CCCCGAGTCTTGCTGACCATGGCCTCTGGAAGCCCTCCGACCCAGCCCTC
GCCGGCCTCGGATTCCGGCTCTGGCTACGTTCCGGGCTCGGTCTCTGCAG
CCTTTGTTACTTGCCCCAACGAGAAGGTCGCCAAGGAGATCGCCAGGGCC
GTGGTGGAGAAGCGCCTAGCAGCCTGCGTCAACCTCATCCCTCAGATTAC
ATCCATCTATGAGTGGAAAGGGAAGATCGAGGAAGACAGTGAGGTGCTGA
TGATGATTAAAACCCAAAGTTCCTTGGTCCCAGCTTTGACAGATTTTGTT
CGTTCTGTGCACCCTTACGAAGTGGCCGAGGTAATTGCATTGCCTGTGGA
ACAGGGGAACTTTCCGTACCTGCAGTGGGTGCGCCAGGTCACAGAGTCAG
TTTCTGACTCTATCACAGTCCTGCCATGATGAGCCCTGTTCCTGCTCATC
ATGAAGATCCCCGCGATACTTCAACGCCTTCTGACTTCCAGGTGATGACT
GGGCCCCCAATAAATCCCGTCTTTGGGTCTCTCTGCCTTTTGCCTGTTTT
TTGTCATGTATGCAAATTTGTTGGGAGAATATAAGTGCTTCTAGTCCCTG
TATTCTCTACATCTTTCAGAATGACTCAGAGCTGCTA
(SEQ ID NO: 151)
MPALLPVASRLLLLPRVLLTMASGSPPTQPSPASDSGSGYVPGSVSAAFV
TCPNEKVAKEIARAVVEKRLAACVNLIPQITSIYEWKGKIEEDSEVLMMI
KTQSSLVPALTDFVRSVHPYEVAEVIALPVEQGNFPYLQWVRQVTESVSD
SITVLP*

An amino acid sequence for the protein encoded by the DCUN1D3 gene that is a regulator of T cell exhaustion/dysfunction resistance is available from the UniPROT database at accession no. Q8IWE4, cDNA NM_173475, with gene location at Chr 16: 20854925-20900358.

(SEQ ID NO: 152)
AGATGAAGAGGAGGCGGTGGCAGTGGTGGAAGAAGAGGCGGCGGCGGCGGGGGTAGGGAGCCTGGAAACGCGA
GCGGGGATGGTAGGTGGTTTGGACCCGCCGGGCCGCCGTCGTTTCCAGAAAGGGTTTGACTGGAGGAACCTCT
GGAGCAGCTGTTGGCTGGCTCCTCTGGCTGATGGCATGTTGAGGTACATGGGCCAGCGGCAGCGAGGGCATCC
AATCCAGAGGGGTCCACTCTAGAGGCCAGGCCACCAGCACCATGGGCCAGTGTGTCACCAAGTGTAAGAATCC
CTCATCGACCCTGGGCAGCAAAAATGGAGACCGTGAGCCCAGCAACAAGTCACATAGCAGGAGGGGTGCAGGC
CACCGTGAGGAGCAGGTACCACCCTGTGGCAAGCCAGGTGGAGATATCCTCGTCAACGGGACCAAGAAGGCCG
AGGCTGCCACTGAGGCCTGCCAGCTGCCAACGTCCTCGGGAGATGCTGGGAGGGAGTCCAAGTCCAATGCCGA
GGAGTCTTCCTTGCAAAGATTGGAAGAACTGTTCAGGCGCTACAAGGATGAGCGGGAAGATGCAATTTTGGAG
GAAGGCATGGAGCGCTTTTGCAATGACCTGTGTGTTGACCCCACAGAATTTCGAGTGCTGCTCTTGGCTTGGA
AGTTCCAGGCTGCAACCATGTGCAAATTCACCAGGAAGGAGTTTTTTGATGGCTGCAAAGCAATAAGTGCAGA
CAGCATTGACGGAATCTGTGCACGGTTCCCTAGCCTCTTAACAGAAGCCAAACAAGAGGATAAATTCAAGGAT
CTCTACCGGTTTACATTTCAGTTTGGCCTGGACTCTGAAGAAGGGCAGCGGTCACTGCATCGGGAAATAGCCA
TTGCCCTGTGGAAACTAGTCTTTACCCAGAACAATCCTCCGGTATTGGACCAATGGCTAAACTTCCTAACAGA
GAACCCCTCGGGGATCAAGGGCATCTCCCGGGACACTTGGAACATGTTCCTTAACTTCACTCAGGTGATTGGC
CCTGACCTCAGCAACTACAGTGAAGATGAGGCCTGGCCAAGTCTCTTTGACACCTTTGTGGAGTGGGAAATGG
AGCGAAGGAAAAGAGAAGGGGAAGGGAGAGGTGCACTCAGCTCAGGGCCTGAGGGCTTGTGTCCCGAGGAGCA
GACTTAGTGGCTCTGTCCCAGGAGCAGCAGCAAGGATCTGCCAGCTGCCCTGCAGCCAACTGAGGAATTGGAC
CATTTTGGAAATTACTGAAGATCCGGATATTTTCTACTTTACACCTTTCTCTGCCTTGTATCTGAAAGGGCTC
TAAAATGCTGTATCATGTTTTAGGCACTTTCTTCATTTTTTTGGTTATTTTGGTTATTTCCTTTTTGGGGGGA
TCTCCCAGAATATTTGAACCTGGTTACATGTTGTGTATCTTTTTTTGAAGCCTTCAGATAGAATAAGCCTGCC
ATTTCTTGCACAAATTTAGGTTTTTTTTTTGTTTTTTTTTGTTTTTTTTTTTTTTTTTTTGGTAGGGGAGGGC
ATAGAGCAGGGCGGGGGGATGGGACTGTTAGGTTGAATTAACATTACAAAATGATACAGTGCCAGATCTCAGT
TTCGCATATTGTTTTTCAGGGCAGGTCTGTACTGTGTGTAGTGCTGTTTACATAGATGAATTTAGGTTGTAAT
AATTATTTTTAAAGATTTACACAGATTTGAATAGCAGTGTTAACTGTTAACCACATTGCATTAATTCCCAGGC
GATTTAGAGCTCTTGGAGAGCCAAGGCCAGCCAAGAGCATTTGTAGTCTGGTGACAACCCCCTTTTAAGCTAA
TTTATCCAGAACCCTGATTTCCCTCACTTCTTGCTCATTCCTTCTTTGACCTATTGCATTTCATGTTGAGTTT
TTCCATCAACATGCTGCACCTGTCAGTCAAGTGAGCATTTTTTAAGAACACATTGTACTGAGAACCACTTAAG
CATTGAATGCGGAGAAAGCAGTGCTACCTCAGTTTTGCTGGAAGTAGACTTCTTTGATAGTTTTCTTTCTTTG
ATGAAGTTTCTGTATTTTCATGTTGTAAGTGGAAATACTTTTTTTTGTTTGTTTGTTTCATTTGCCTTGGAGC
CAAAGTTTCTGTTCCTGGTGGTCGGGAAACTGCCTGCCGGCCAACTGACTTGAAGGAAAACTGTGGTATGGAG
CTCTGCTTGAATTTTTTTTTTTTTAATATTTTTATTTTTTTCTTTGAATATCATCAGCTTACTTGTCTGGCAA
GGGCAGAAGCCTGGGGTTGGCCTGAACTCTGCCAAACAAATATCAAAGTGTATTTAATAGTTAAATTTGTGCC
CTTTCCCTTCTTGCTGCACCCATGTTGTCACTTAACCCCCAGGAGTTATTTATTATCTTTTTGTTAAAGTCAG
GCTCATTTGGGGTAATGTGATGACTGTTTAGGTTTACATGACCCTCCTCTCCTTTCCCTACCCCCAAATATGT
ATATATACATATATAAAATATGTATATATTTTACCTATATAAAATATATATATATACACATATATGTATCTAT
ATTCCTTTGTTTCTTTGCCTGCTTATACTGGCCATAAAAGAGGGAGCTGCCTTCAATGTATAAAGTATAAGAA
GAGTGCCAGGGAATGCCATAATGGAGGCTTTTGGATCTGAATTTGGACCATTTCACTAAAGAGAACATGAGTT
TGCTCAGCCCTTTCCTCACAAGAGGGAGGGCCCCGGTTCCCCAGACTTCTCCACGCGCTGGCTCCATAAAGGC
CAGCTTTGGCCAGGCTGCCACAGGGGCCTGAGGAGCTCACTCTGGGCCTACCTGGTTTCAGTTAGAGGGTCCT
CCTGTTATTTTTCCATTTAAAAAGTATGTCCTCAGAAAACTGTACTGGAAGGATGGGTGGCAGGAACTTGTAT
AGTTCAGCTTCCAACACTTTGGAACAGATTAAAAAGGGAATCTTTTAAATAAAAACGTATAAAAATATTTATA
CTCTTGAGGTAATGAGACTTTGTCTGTTAAACATTTGGAGGTTTCTCCCTTAACCTAATACCCCGCTGCGTCC
CAGGTATGTTTCTCAGCCTGGGCAGGAATTGCATGTTTTGAGTTCCATTGTGAGCAGCCTTACAGCTCTGGGC
TCCCATCTTAAGGATTATTAAAGAGTTTTGGCAGTTGGTATTTTTCTAAAACTGATATTGACCATAGGGAACA
TACTTGGAATTTGTTTTTTTTTTTAAATCCTAAACCTGACAGTTCCTCCTTCAGAGGGGCAAATGGGAAACTT
TAAGATCACCCACGGTATAGTACTGCTAGAAAGCTGAAGGGGGTCTTGGGCCAGATGCCTGGCTTCTGTTCTA
GCTAAATAAGACTCTTGAATAAGTCTCCAGGGGTCCCAGCAGGTTCTGTGGGATCAGTTGTAGCTGGGTACCC
AGACTGAGGTTTTAAAAAGACTGCTTTTTCATTAAGCCAGTTTGCAACTCATTTTCCCCGGTTCTGGATTGGG
TAGTGGTTGACTGAGGGAAAAGCCAGGCTGCTCTTGGCCAGATAAGGCCCTTTCCATCCCAGATCAGAGTGTA
TCAGAGAAAGCTGCACAAAAGAGGCAGGCAGCCCAGGACCCTGAGTACTCTGGAGAAACTAGGTTCCTTCCCC
ACCCTTTAAGAAGACATTCCGTGCATTAGATGTACTAGAGTGGATGTATTTTGTTGTTTTTTAAATTAACTAT
TTAGCCTCCTCATCCCCCACCAAAAAAGCCATTTAGTTATTTTTTGGTTATATTGATCCATTTGCAAATGAGA
AGCCAGAAAAGGGAGCAGTCAGGGAGGGACTTACAAGTTCCTTTCAAGTTTGAGTACTTGATGCTCAGCAAAG
ATTTCAAGCTTCTGCAGTAGCTCTGGGCCAATGCTTGACTCTTTCATGACCACAAGAAATGCAGTTTTTCTGC
AAAGGGATCCAAGGTGAGGTGTGTGTAGGGTTTGAAGTTATACTTCTGGGAAGTGAAAAGTCTTGTTCCTTTC
AACCTAGAAATAGGTTTGCCACTTAATGAGTGAGCAGTAAGTCTGTGTAAGAGGCTGAATGCATGCCCCTCAG
ATAAGCCAGTACACTCCTTGCTTAGCAACAGAACATCAGGGTGATGTGGAGAGGGGCAGGATGTGGACGCCAC
TTTGGAAATCGGCAACATCTGAGGGCAACAACAAACAAGTGTGTTGGGAAATAAGAAATAACTCAGTTTTGAC
AACTGGCTTTGTCAGCTTTTGTGATGTTTCTTTAGCAGTTTATTGGAAAGATGGTATGAGATGACGTGCTGCT
TCATTGAATTGCTCTTTCCCCCATCTTTGCCAAATCTCAATGTATCGTTCTTAACCCCACCTCCTGTAAGGGG
CTTTGCTATGCTTCAGCTGGTTGTCTCAGCAGCTGAAGTGCTGCCCACCTGTGTGAGTTGGGTCCAGGAAACC
ATGTCTGCCCTTCTGATAAGGGAAGATGAATCTAGAGCTGGGTGAAGATCTAAATTTTAACCAAACCCCTGGG
CCCAGGAAAATAACAATTGAAAATGTACAAGGCAGTGTTTTCAATATTAAACTTCCCCAAGGAAAGCACAAAC
TAGTCTTTTTGGAAAGGGAGAAAGGATTAAGCCACACAGTATTAGTCTTTGAAGCAGTACTGGTCTCTAGGGG
CTGGTGCCAAAATGGAGTCCCATAGTAGTTACACTCGATGGCCTCATGTACTATATACTGTGCCAAATTGTAT
TAAACAGTGGTGGGGAGTTACTGGGATAAGAACTTGTCTAAAAGTTTACAAACCAAAACAGATCTGTTAGGTT
GGTGCAAAAGTAAGTTTTTGCCATACTTAATGTATTGCCATTAAGTATGGCAAAAACCACAATTACTTTTACA
CCAACCTATGTATTTAAGAATGTTTGGTTTGCCAGATTCCAAATGAGGTCTTCAGTGCAGCAAAGCCCAAAAG
GTGTAGACTCAGTTATGCAATTATAAGGTTAAGGCGTAGAAGAAAGCTGCTGCTAGGTTTTTGTTGCATTTTA
CTTGACTGCTCTGCTGTTTTTCTTGTCTCTCATGTTTGGTTAGCTATGACTTGAGCATCTTGGTAACTGACAA
AGGTCTTCCTTGGGGGACTTGAACATCTTGGTAAATGACAGGTCTTCTTGGAGGACTCCAGCAGTATCTTGTT
TAAACGACTGAAAGGACTATTAAGGTTGTTGAATTGTGTTAATTGGGACTCATTGAGGAAATGCGACATTGAT
CCTCCTCTTATTCCACAGTGTGTTTTCTGATCATATAAAGAAGGTTCCGAACCATCCATCCCCCTCAGAGTTT
ATTCCCCTGGTAAGCTGTAATTGCATATCCAGTTTAAACTGGACTGGGACTGCATGTTGGTGAGGATCGGCAG
GGGTTTTCCCCCTTTTCGAAAGATGAAATAGATTCTTGAGCACTGGTTGCAGAAGCCAAAATAGTTCAAATAG
CTTTGCATAACCATTGGGTTCTGCTTCTGATTCAGGTGCTGGGCATCATGTCCTCCCTATTCTTCTCTTCTTG
GAAACCCAGCCTATCTCATAAATACCTACTTCCGCCACCCATCCAACCTCCCTGCTCCTTTCAACACAAATCT
TGGATATTGCCAAAGGAAGCCATTCAGCAGCTGCTGGGGTTTTTCATCCCCCTGACATGCATACATTTGCTCT
GGGAGAAGTGTCTTCCCTCTGACCCTGGTCCCCAGCTCCGTCTGTGCTTAATTGCTCTTACCTGTTGCACTAA
TGAATATGACTAGGTTTTAAAGGGGGAATGTGAAGCAATAGGCACATGGGGCTTGGATGAATTGGTCCCACAG
ATATACCCTGCCTTAAGCCGCTGAGGTGATGAGTCCACTGCTCATGTGACCCTCCACCTTTGTGGATCCCTCT
TGGTTTGTGACCAGTGTGTCTGTTTGTTGAGGTTGTACAAACTTGACAAAAGTTAATACTTTTGTTTGTATTT
TCTGCACTGTTGCACTCTCCAAATGGCCCCTTGAGTATTTTTATTGACTTGTTACACACATTTTTGTCTTTGA
TGTCTACATTTTTTCCTTTAATGTTTTTTATTTGGAAGGTTACCTGCTGTTGGATTTAATAAATTTGTTTACT
TGAA
(SEQ ID NO: 153)
MGQCVTKCKNPSSTLGSKNGDREPSNKSHSRRGAGHREEQVPPCGKPGGDILVNGTKKAEAATEACQLPTSSG
DAGRESKSNAEESSLQRLEELFRRYKDEREDAILEEGMERFCNDLCVDPTEFRVLLLAWKFQAATMCKFTRKE
FFDGCKAISADSIDGICARFPSLLTEAKQEDKFKDLYRFTFQFGLDSEEGQRSLHREIAIALWKLVFTQNNPP
VLDQWLNFLTENPSGIKGISRDTWNMFLNFTQVIGPDLSNYSEDEAWPSLFDTFVEWEMERRKREGEGRGALS
SGPEGLCPEEQT*

An amino acid sequence for the protein encoded by the DDA1 gene that is a regulator of T cell exhaustion/dysfunction resistance is available from the UniPROT database at accession no. Q9BW61, cDNA NM_024050, with gene location at Chr 19: 17309518-17323298.

(SEQ ID NO: 154)
AAGTTACTGTGAGGCGGCGGCTAAGAAGGCGGCTCTGGTGGCGGCGGTGGAGGCTGAGGCGGCGGCCGAGGCG
GCGACGGAGGAAACAGAAGATGGCAGATTTTTTGAAAGGACTGCCTGTCTACAACAAAAGCAATTTTAGTCGA
TTTCACGCGGACTCCGTGTGCAAAGCCTCGAACCGACGGCCCTCAGTCTACCTGCCTACCCGCGAGTACCCGT
CTGAACAGATCATCGTGACAGAAAAGACAAACATCCTCCTGCGCTACCTGCATCAGCAATGGGACAAAAAGAA
CGCTGCCAAGAAGAGAGACCAGGAGCAAGTGGAGCTGGAAGGCGAGAGCTCCGCACCTCCCCGCAAGGTGGCG
CGGACCGACAGCCCAGACATGCACGAGGACACTTAAGACTCTCAACTCCACAGGCGCCTCCTGCCAGGTCTGC
TCCTCGGTCGCCCACCCGCCTGCCCGCCATGTGTAAGCACCCCGCCCGCCCGCCTCCCTGCCGGCCCATCCAC
ACCCTGCGTCCACACCACTTCCAACCTCATAGGAGCCGATGTATTTATTTTCCTTGAGTTTTTATTTATGCTG
TAACCTGTATCAAGCGTTGGTTAAAGGGGACATCAGACCCAGTAGTGTGATGTTGGTAGATGCTTTTTAAAAA
AAACAACATTGTCCCCCCGACCCCCGCCTTCCATCGGGCCAGTTCCCCGATTCCTGCCCCCAGTTCTCCAGAG
AACCAGAGTGTGTCTGTGAGAGTCTCTAGCGGGGGCTTTACTGTGGCCGGGCGACAGGGGCGGGCCCGGGGTG
GCCTGACCTACCAGGACAGCCGAGTGGCCTTCTCCCCCCCAACACCGGTCCAGGCCATTGAGACTCGGTCTTG
TCCCACGCTTCGCCCGGAACTTTCCCATGCCCAGGCCTCACTCAGCGTGCACGCACGTTGGGGAGAAGTCGGC
CCTTGGGATCTTTCTCTTGAGTCATTTTATTTTTATCATGGACTAGTGCGTGCTCCGTGTCCACCCCAATAAA
AGGGTCTTTCCTACTTGACCTCGCGTCTTTGCTCCACACACCTTGGCTGCAGAGGACGGAGCCCCTGGGAGTC
ATCCCTGTTGTCACCAAGAGGAGTGAGAAGGGCCCCCCACGCCAGGGGCCCCAGGACCGGCTGGATTCTCTCG
TGGGCTTGCTGAGGCCACCATGTTCAAGGCCAAGACCTGCCTGGACCATCCCCTCTGCCTCTCCTGTCAAGGT
GACCCCAAGGACAGGTCATTCCTGAGAGACGGTCCATGGTGCCAAGGATGGAGCGGGGTCAGCCCCCTGCCTG
GGATACCGCCAAGCAGGGCAGCGCACCTGTCAGACACCGCAGAGATGGCCGGCCTCATCCGGGGGCCTGGGGT
CTCTGGCCTCTGCTGCTCCCCTGAGGCATGGTGACAGTACAGACGGCATCTGGCCGGTCTTGCTGTCCTAGGT
GGTGGCCTCACCCTTGCTGGGTCATAGAGGAGCTGTACAGAAGGTTGGCACCTGGCAGTTGGCATGTGCCATG
GCCGCTCATGGGGACCCCTCTTCCGCCAGCTGGGCTGGGTTTGGGGCTGAGCTTGGGTATGTAGGGGTGTTCG
GGCTCTGGGCAGAAGGTTTGTGGCCCTGACCACTGAGCCTGGGGCTGGGGGAACACCGCTTCTCCCCTTATTG
CCCCCACTGGTGGTGGTGGGGATCTCAGTTTTCTTTTGTTTTGTTTTGTTTGTTTGTTTGAGTTGGGGTCTTG
TACTCTTTCCCAGGCTGGAGTGCAGTGGCGTAATCACAGCTCACTGCAGCCTCGACCTCAAGCCTTCCTCCCA
CCTCAGCCTTCCAAGCCGCTGGGACCACAGGCACCCACCACCATGCCTGGCTAATTTTTTTTTTTTTTTGAGA
TGGAGTCTCGCTGTGTCTCCCAGGCTGGAGTGCAGTGGCAAGATCTCAGCTCACTACAACCTCTGCCTCCCAG
GTTCAAGTGATTCTCCTACCTCAGCCTCCTGAGTAGCTGGGATTACACGCGTGCGCCCCTACACCCAGCTAAT
TTTTCTATTTTTAGTGGAGACGGCGTTTTGTCATGTTGGCCAGGCTGGTCTCGAACTCCTGACCTCAGGTGCT
TCGCCTGCCTCGGCCTCCCAAAGTGCTGTGACCGCACCTGGCTGCCTGGCTAATATTTTAAAAATTTTTTGTA
GAGACAGGGTTTCACCATGTTGCCCAGGCTGGTCTCAAACTCCTGGGCTCAAGTCATCCACCTTCCTCAGCCT
CCCAAAGTGCTGGGGTTACAGACGTGAACCACCGTGCCTGGCAGAGGTCTCTGTTTTGAGAGCTGAGTCTGAG
GGCGTTGGGATGGTGTAGGGTTGGGCCACAGAGCTAGCAGACACTCCGGGCTATGGAGGGTCCGCCTGCCCCA
GATTCCACTGTGGAGGGCCCTGCCTGTGTCAGACCCTCGGGCCTAGGCGGCCCATCTCTCCCAAGGACGGAGA
GTGCCAGGGTGGGCTCCCGTCCTACACAAGGAGTGGCTGACCAGGCCCAGGCACAGCACAGCCACCCTGCTGT
ACCTCCCAGCTCCCGGCAGTGTCTACACCACAGTGATGGTGCTACAGGGGCCATGTGGTCACAGGACAGGTGG
TCAGGAAATGCACACTTGACTGCGGTCGATGGTGCCGGGTCCGGCTGAATGCCCTGGAGGCTCCCAGGCCAGC
TGGGGAGTCCCAGTCCCCAGGGTTGGTGTGACCTGGGCAGGCCTGCGGTGTCCATCTGTGAATGACTGAGATG
GTGGGACTTCAGCTTCCAAGGCCAGGGGCCAGAGTGGGAGCAGGGGGCACTCCCGCCTTTCCCCTCGGCCTGT
CTAGACCTCTCTGGCTCCCAGTTTCTGCTTTGAGTGGCTGGGGGCTGTCTCCTGCCTGTGCTGTCTGCCCGTC
GCTCTGCAGGGCTGCTGGGCGGGCACCTCCTTCCCCTCCCCTACCTCAGCTCCCTCCTTTGGGGTCTGAGGGC
TGTGCCCCAGCTTGCCTCCCTGTGGCCTCCCCCTAGAACCCCTGGGTCTGTCGGGACATGCCCCACCTGGCAC
ACACACCCTGTGTAGCTCCCACTGCCTGTGGGAGGACGCTGGGCCCCTGAGCCGGCCTCTGTCTACCCCTCCT
GCCTTTGGGGTCCCCTTTACCCCACTGTTCCCCCAAACTGCCTTCCACCTTCTCCGCCTTTGCACAGGCTGTT
CTCCTGCCTCCAGTGAACTCTGACTTCTCCACAGCCTCCCAAAGGGCCCCTTGCTCACACTGGCCCAGCCCTG
CAGCATGGCCATGTTGGTCTCTGAGCCTTAAGCCTTTGGATGTGGACACATTTGTCCTCTTCCTGAAAAATGT
GAGGGTCTGAGAGGCCACGTTCCTGTTCCTGAGTCATACGACAGAGTCCCAGTGACTTGGGTCTGATGTCTGT
CCCACAGTGGGACATCCCTGCTGCATCCCAATGCCACCCCGTGTCCTGGGGACACCCAAATGTGCCATTCTCT
CAGTATCACAAGTCGGGGACTGCAGGAGGCTCAGGTGCCAGGTGCCTTCTGATGTTCATGCCACAAGCTCTTC
CCACTGTCTCTGGCTCCCCAGACGATGTTCCAGGAGGCGGGTGTGCCAGAAGGGGCCACGTCTTGCAAACCAC
CGCGCTGTCCTCTTTGAGAAGGAGTCTTACTCAGGACTGGGGCCTGTGCACACATTGTCGCCTCTTTTCAGCA
CTTAGAGATTCCTTCCTTTGTCTAGTGGCTGAAGCCAGGGCTGAAGTTGGCCTCCAAATCTGGGCCGTCTCAG
AGGCGGCGCAGCCTGGAGTTTTCCATCTGTGGCCAAGACCCAGTTTTTGGGAGGAGGCCCTCATGGGTCAAGC
CAGCCTGTACCAGGATGGGGGGAGGGGGTTCCCAACCTAGGCCCCAGCCACCCAGACTCCCCCACCCCCACTC
CCTTTTCCACTGCTCTGACCTCGGGCACTGTTGAAATATAGTTTTTATTGCATTTCTGCCGTTTTACAAAAAT
ATGACAAAATAAATTAAAAACAAATAAA
(SEQ ID NO: 155)
MADFLKGLPVYNKSNFSRFHADSVCKASNRRPSVYLPTREYPSEQIIVTEKTNILLRYLHQQWDKKNAAKKRD
QEQVELEGESSAPPRKVARTDSPDMHEDT*

An amino acid sequence for the protein encoded by the GABARAPL1 gene that is a regulator of T cell exhaustion/dysfunction resistance is available from the UniPROT database at accession no. Q9H0R8, cDNA NM_001363598, with gene location at Chr 12: 10212458-10223128.

(SEQ ID NO: 156)
GGCTCGCGGGGTGACGTCACAGCCCGACGCGCCACCCAGCTGTTTTTGTG
CTCCCAGCTCTAGCGAAAAGCCGCCGGTATTTCTCCATCTGGCTCTCCTC
TACCTCCAGGCAGGCTCACCCGAGATCCCCGCCCCGAACCCCCCCTGCAC
ACTCGGCCCAGCGCTGTTGCCCCCGGAGCGGACGTTTCTGCAGCTATTCT
GAGCACACCTTGACGTCGGCTGAGGGAGCGGGACAGGGTCAGCGGCGAAG
GAGGCAGGCCCCGCGCGGGGATCTCGGAAGCCCTGCGGTGCATCATGAAG
TTCCAGTACAAGGAGGACCATCCCTTTGAGTATCGGAAAAAGGAAGGAGA
AAAGATCCGGAAGAAATATCCGGACAGGGTCCCCGTGATTGTAGAGAAGG
CTCCAAAAGCCAGGGTGCCTGATCTGGACAAGAGGAAGTACCTAGTGCCC
TCTGACCTTACTGTTGGCCAGTTCTACTTCTTAATCCGGAAGAGAATCCA
CCTGAGACCTGAGGACGCCTTATTCTTCTTTGTCAACAACACCATCCCTC
CCACCAGTGCTACCATGGGCCAACTGTATGAGGTAATGGTTCTGGTTGCA
CAATACTGGATGCCGTCCAGTGCAGTCTGGCATCCTCTAGCCCTTGTTCT
AGATGCGTTGATAACACATCTGAGAAGTGGGGCAGAAGGTGTTATTTATC
CGGATCCTCTTACATATGGCAGTGTAAGGCTCTGAGAGATTAGATACCTC
TTGTTTTTTAGGGATTAGCCACTCTACTAGATTTAATCCTTCTGAAATCT
TGTCTGACTATAGTCTTGTCTGACTATAGTGTTTTAATGCTGGACTGTTT
ATTGA
(SEQ ID NO: 157)
MKFQYKEDHPFEYRKKEGEKIRKKYPDRVPVIVEKAPKARVPDLDKRKYL
VPSDLTVGQFYFLIRKRIHLRPEDALFFFVNNTIPPTSATMGQLYEVMVL
VAQYWMPSSAVWHPLALVLDALITHLRSGAEGVIYPDPLTYGSVRL*

An amino acid sequence for the protein encoded by the KDM5C gene that is a regulator of T cell exhaustion/dysfunction resistance is available from the UniPROT database at accession no. P41229, cDNA NM_001146702, with gene location at Chr X: 53176283-53225422.

```
  (SEQ ID NO: 158)
CTTGTTCCTCCGCCGTTGCAATGAACTATTTTCTCTCAGTCCGGGGTGGTACTTTTGAGTAACCCCTTCCAAA
TAGAAACCCATACCAGCCTATTTAGCTCGGTCTCCACTATATGAAGGTTTCCTAGGGCTTGGTGTGACGCAAC
GTATACGAGGCTCGGAAGGACACCCCGCGGAAGGATCCGGTTTGTTGTGGTGTGGGGAGGGGAGACGCTGACA
AACCAAGATGGCGGCGGCGGCGCTGAAGGCCGCGGCGTTTGGGAGGTAACTGTGGTGGCGAAGGCTGCGGTAG
TGGGGAGGCAACCACACAGTTTGGAAGAAACGGAGCGGGACGAAGAGGCGGTAGCAGTAGAGTCAGCCTGAGA
CTCTCAGAGCACGACGGCCACACGCCCCCTTAGGCCCTCGGCGGGCGGCGGCTGCCCCGCTTAGGGCCTAGCC
TCCGAGCATTGCCTCGGCTTCAAACAGCGGCGGCGCCATGAGTCCTTAAGGGCGGTCCAAGCCTCCCGATCCC
TGGCCCAGACCTCGGGCCCACCATGGAGCCGGGGTCCGACGATTTCCTACCGCCACCGGAGTGCCCGGTGTTC
GAGCCTAGCTGGGCCGAGTTCCGAGACCCTCTTGGCTACATCGCGAAAATCAGGCCCATCGCAGAGAAATCGG
GCATTTGCAAGATCCGCCCACCCGCGATTGTGGTGGAGGAAGGTGGTTATGAAGCTATCTGCAAGGACCGTCG
GTGGGCTCGGGTAGCCCAGCGCCTCAACTATCCACCAGGCAAAAATATTGGCTCCTTGCTACGCTCCCACTAC
GAACGCATTGTTTATCCCTATGAAATGTACCAGTCTGGAGCCAACCTTGTGCAGTGTAACACACGTCCATTTG
ATAATGAGGAGAAGGACAAGGAATACAAACCCCACAGCATCCCCCTACGACAGTCTGTGCAGCCTTCCAAGTT
CAACAGCTATGGCCGGCGGGCCAAGAGACTGCAGCCTGATCCGGAACCCACAGAGGAAGACATTGAGAAGAAT
CCAGAGCTGAAAAAGCTACAGATCTATGGGGCAGGCCCCAAGATGATGGGCCTGGGCCTCATGGCCAAAGACA
AGACTCTGCGGAAGAAAGATAAGGAGGGGCCTGAGTGTCCCCCCACAGTAGTGGTGAAGGAGGAGTTAGGTGG
GGATGTGAAGGTGGAGTCAACATCGCCTAAGACCTTCCTGGAGAGCAAGGAGGAGCTGAGTCACAGCCCAGAA
CCCTGCACCAAGATGACCATGAGGCTACGGAGGAACCACAGCAATGCCCAGTTTATTGAGTCATATGTCTGCC
GGATGTGTTCTCGAGGGGATGAGGATGACAAGCTCCTGCTGTGTGATGGCTGTGATGACAACTACCACATCTT
CTGCCTGCTGCCTCCTCTGCCTGAGATCCCCAAGGGTGTCTGGCGGTGCCCAAAGTGTGTCATGGCGGAGTGT
AAGCGGCCCCCAGAAGCCTTTGGCTTTGAGCAGGCTACCCGGGAATACACTCTGCAGAGCTTTGGCGAGATGG
CCGACTCCTTTAAAGCTGACTACTTCAACATGCCCGTGCATATGGTGCCCACAGAACTTGTGGAGAAGGAGTT
CTGGAGGCTGGTAAATAGCATTGAGGAAGATGTGACTGTTGAGTATGGAGCTGACATCCATTCCAAAGAATTT
GGCAGCGGTTTCCCTGTCAGTGACAGTAAACGGCACCTAACCCCCGAAGAGGAGGAGTATGCTACCAGTGGTT
GGAACCTAAATGTGATGCCGGTGTTGGAACAGTCTGTACTGTGCCACATCAATGCAGATATCTCTGGCATGAA
GGTGCCCTGGCTCTACGTGGGCATGGTCTTCTCAGCCTTTTGCTGGCATATTGAGGATCACTGGAGTTACTCC
ATTAACTACCTCCACTGGGGTGAGCCGAAGACCTGGTATGGGGTGCCCTCACTTGCAGCAGAACATTTGGAAG
AAGTGATGAAGAAGCTGACACCTGAACTATTTGATAGCCAGCCTGACCTCCTGCACCAACTTGTCACCCTCAT
GAATCCCAACACCCTCATGTCCCATGGTGTGCCAGTTGTCCGCACAAACCAGTGTGCAGGAGAGTTTGTCATC
ACCTTCCCCCGTGCTTACCACAGCGGCTTCAACCAAGGCTACAACTTTGCCGAGGCTGTCAACTTTTGCACTG
CTGACTGGTTGCCTGCTGGGCGCCAGTGCATTGAGCACTACCGCCGGCTCCGGAGATACTGCGTCTTCTCCCA
TGAGGAGCTTATCTGCAAGATGGCTGCCTGCCCAGAGAAGCTAGACCTGAACCTGGCGGCAGCTGTGCATAAG
GAGATGTTCATCATGGTGCAAGAAGAGCGGCGTCTACGAAAGGCCCTGCTGGAGAAGGGTATCACAGAGGCTG
AGCGAGAGGCTTTCGAGCTGCTCCCAGATGATGAGCGCCAGTGTATCAAGTGCAAGACTACGTGTTTCCTGTC
AGCCCTGGCCTGCTACGACTGCCCAGACGGCCTTGTCTGCCTTTCCCACATCAATGATCTCTGCAAGTGCTCC
AGTAGCCGGCAGTACCTGCGGTATCGGTATACCTTGGATGAGCTTCCTGCCATGCTGCATAAGCTGAAGGTTC
GGGCTGAGTCCTTTGACACCTGGGCCAACAAAGTGCGAGTGGCCCTGGAGGTGGAGGATGGGCGGAAGCGCAG
CCTTGAAGAACTGAGGGCACTAGAGTCTGAAGCCCGTGAGCGGAGGTTTCCTAATAGTGAGCTGCTGCAGCAA
CTAAAGAACTGCCTGAGTGAGGCAGAGGCTTGCGTGTCCCGAGCTCTGGGACTGGTCAGCGGCCAGGAAGCTG
GCCCCCACAGGGTGGCTGGTCTACAGATGACCCTGACTGAGCTCCGGGCCTTTCTGGACCAGATGAACAACCT
GCCTTGCGCCATGCACCAGATTGGGGATGTCAAGGGTGTTCTGGAACAGGTGGAGGCCTACCAGGCTGAGGCT
CGTGAGGCCCTGGCCTCACTGCCCTCCAGTCCAGGGCTACTGCAGTCCCTGTTGGAGAGGGGGGGGCAGCTGG
GGGTGGAGGTGCCTGAGGCCCAGCAGCTCCAGCGGCAGGTGGAACAGGCGCGATGGCTGGATGAGGTGAAACG
CACACTGGCCCCCTCAGCCCGAAGGGGCACCTTGGCTGTCATGCGAGGACTGTTGGTCGCGGGTGCCAGTGTA
GCCCCTAGCCCTGCTGTGGATAAAGCCCAGGCCGAGCTGCAGGAACTGCTGACCATTGCTGAACGCTGGGAGG
AGAAAGCCCACCTCTGCCTGGAGGCCAGGCAGAAGCATCCACCAGCCACACTTGAGGCCATAATCCGTGAAGC
GGAAAACATCCCTGTTCACCTGCCCAACATCCAGGCTCTCAAGGAGGCTCTTGCTAAGGCCCGGGCCTGGATT
GCTGATGTTGATGAGATCCAAAATGGTGACCACTACCCCTGCCTGGATGACTTGGAGGGCCTAGTAGCTGTGG
GCCGGGACCTACCTGTGGGGCTGGAGGAGCTGAGACAGCTAGAGCTACAGGTACTGACAGCGCACTCCTGGAG
GGAGAAGGCCTCCAAGACCTTCCTCAAGAAAAATTCTTGCTACACGCTGCTGGAGGTTCTCTGCCCATGTGCA
GATGCCGGCTCAGACAGCACCAAGCGCAGCCGGTGGATGGAGAAGGAGCTGGGGTTGTACAAATCTGACACAG
AGCTGCTGGGGCTGTCTGCGCAGGACCTCAGGGACCCAGGCTCTGTGATCGTGGCCTTCAAGGAGGGGGAACA
GAAGGAGAAGGAGGGTATCCTGCAGCTGCGTCGCACCAATTCGGCCAAGCCCAGTCCACTGGCATCATCGAGC
ACGGCCTCCTCTACAACCTCTATCTGTGTGTGTGGGCAGGTGCTGGCTGGGGGGGGAGCTCTGCAGTGTGACC
TGTGTCAGGACTGGTTCCATGGGCGGTGTGTGTCAGTGCCTCGCCTCCTCAGCTCTCCGAGGCCCAATCCCAC
CTCATCCCCACTGCTGGCCTGGTGGGAATGGGACACCAAATTCCTGTGTCCACTGTGTATGCGCTCAAGGCGC
CCGCGCCTGGAGACCATCCTGGCACTGCTGGTAGCCCTGCAGAGACTGCCTGTGCGGCTGCCCGAGGGCGAGG
CCCTGCAGTGCCTCACAGAGAGGGCCATCAGCTGGCAAGGCCGCGCCAGGCAGGCTCTGGCCTCTGAAGATGT
GACTGCTCTTTTGGGACGGCTGGCTGAGCTCCGCCAACGGCTACAGGCTGAACCTAGACCTGAGGAGCCTCCT
AACTACCCTGCAGCCCCTGCTTCTGACCCCCTCAGAGAGGGCAGTGGCAAGGATATGCCTAAGGTCCAGGGCT
TACTGGAGAATGGAGACAGTGTGACCAGTCCTGAGAAGGTAGCCCCGGAGGAGGGCTCAGATCTGGAGCTGCT
GTCCTCGCTGTTGCCACAGTTGACTGGCCCTGTGTTGGAACTGCCTGAGGCAACCCGGGCCCCCTTGGAGGAG
CTCATGATGGAGGGGGACCTGCTCGAGGTGACCCTGGATGAGAACCACAGCATCCCTGAGTCCCTTGACTTTT
GTATTCTGACTCCAAGGTATTGTTCAGACCTCAGCTCCTGGGGGCCGGCCCCTGGAGTCTTCCCTCCCTGGTA
GCCTCTAACCAGCATTCCCAGACACCTGAGGCAGATAGATGGATGGGCTGGTGGGCAGGGGGGTGGCTGGGGC
TGGGCCATCACCATTCCAGAGACAAGGCCAGTGTATATGCAAACTGGGGGACTCTCCTCCCTTCTCTCCCCAG
TTCTGGTCCTGGCCAGGCCATGCTACACTAACCCCTGCCCCCACTCTCCTCCCCTCTTTTCCTTCCTTCCTAC
CCCCTTCTCCCTCTCCCTTCCCCTGACTGTTCCACCCAGGAGGAGGAAACTTCACATAGCCGTGCTCACAGTT
TTTTATTTTAAAGGAATTTGGCTGGGGAGCTGAACAGGGCTCCCTGTGATCTGAAGAAAGCTTTTGGTGCTTG
TCCTCACAACCACCTCAGTCCTCCCTCCCTGTCCTCCCCTGTCTCCTTTCCTCCTCCTGGGTTCATGTTGTAA
TAAAAGAAGATTGTTGGTGTGTAATTAATTTGTTCAAAAGAAAAAAAAAAGCAAAACAAGAAACTTGGTTCCA
ACTGAAGCCTATTTTAATTTTATTTTATTATTTTCCTCTTGTTAGAAATAAAACCCTTAGAAACATTTTTTGG
AAACATGTTTCTGAAGTGCATTTCTCTTAGACGGGGAAGACAGTGCTTCCATCACCAGTCAGTGGAGCAGCTG
TAGTGCTGGCAGGAGCACTAGGGCTGAAGCCAGGGAGCTCTGGGTTCTGAAATCAAGTTCCACCAGCTTCTTA
ACCTTGGGCAAGACATTCTGATCCTCCCCCGACTCTAACCCCAAGCCACTTTCCCTCAAGTCCAAACAGATGG
GCTCGGGCCCCCTCACCCAAACTCCTACACTACAACCTCCCCCAGTTGATGCCCTCCAGTTTATCCTCCTCAA
GCTCCCAAAGGGATTTTTTTGTTAATGGCTTTATTGAGATATAATTTACATACTATAAAATTCACTTGTTTGA
AATGTACAATTCAGTGATTTGGGGGGTTTCTAAAGTGTAGATCTGATTATATACTGCTTAAAACCTTCCATGA
TGCTTTATTGCCCTTAAGATTATACATAGAATCTCGTTCTTCCTAGAATGTTTTCCTGTCTAACCCATCTACT
GCATAATTCCCTGATTTTTCTCCACAGGTTATGCCCCTGATGAATGGGGACTAAAGCTCAGATGTGTGGTCAT
TTGCAACCCCTGAGTTCACTTTTTTAAGACAGCTTATTGATATATATAATGTACATACCATACAGTCACCCAC
TTAAAAGTATACAATTAAATAGCTTTTAGTATATTCAGAGTTGTGCAGCCATCATTCGAAAAAAAATTTCAGA
ACATTTTCATCACTGCAGAAAGAAACTCCATATCCCTTAGGCGTCCTTTACCCCTCATCCCATTTGCCTATTC
TGGACATTTTACATAAATGGAATCGTATAATATGTGA
  (SEQ ID NO: 159)
MEPGSDDFLPPPECPVFEPSWAEFRDPLGYIAKIRPIAEKSGICKIRPPAIVVEEGGYEAICKDRRWARVAQR
LNYPPGKNIGSLLRSHYERIVYPYEMYQSGANLVQCNTRPFDNEEKDKEYKPHSIPLRQSVQPSKENSYGRRA
KRLQPDPEPTEEDIEKNPELKKLQIYGAGPKMMGLGLMAKDKTLRKKDKEGPECPPTVVVKEELGGDVKVEST
SPKTFLESKEELSHSPEPCTKMTMRLRRNHSNAQFIESYVCRMCSRGDEDDKLLLCDGCDDNYHIFCLLPPLP
EIPKGVWRCPKCVMAECKRPPEAFGFEQATREYTLQSFGEMADSFKADYFNMPVHMVPTELVEKEFWRLVNSI
EEDVTVEYGADIHSKEFGSGFPVSDSKRHLTPEEEEYATSGWNLNVMPVLEQSVLCHINADISGMKVPWLYVG
MVFSAFCWHIEDHWSYSINYLHWGEPKTWYGVPSLAAEHLEEVMKKLTPELFDSQPDLLHQLVTLMNPNTLMS
HGVPVVRTNQCAGEFVITFPRAYHSGENQGYNFAEAVNFCTADWLPAGRQCIEHYRRLRRYCVFSHEELICKM
AACPEKLDLNLAAAVHKEMFIMVQEERRLRKALLEKGITEAEREAFELLPDDERQCIKCKTTCFLSALACYDC
PDGLVCLSHINDLCKCSSSRQYLRYRYTLDELPAMLHKLKVRAESEDTWANKVRVALEVEDGRKRSLEELRAL
ESEARERRFPNSELLQQLKNCLSEAEACVSRALGLVSGQEAGPHRVAGLQMTLTELRAFLDQMNNLPCAMHQI
GDVKGVLEQVEAYQAEAREALASLPSSPGLLQSLLERGRQLGVEVPEAQQLQRQVEQARWLDEVKRTLAPSAR
RGTLAVMRGLLVAGASVAPSPAVDKAQAELQELLTIAERWEEKAHLCLEARQKHPPATLEAIIREAENIPVHL
PNIQALKEALAKARAWIADVDEIQNGDHYPCLDDLEGLVAVGRDLPVGLEELRQLELQVLTAHSWREKASKTF
LKKNSCYTLLEVLCPCADAGSDSTKRSRWMEKELGLYKSDTELLGLSAQDLRDPGSVIVAFKEGEQKEKEGIL
QLRRTNSAKPSPLASSSTASSTTSICVCGQVLAGAGALQCDLCQDWFHGRCVSVPRLLSSPRPNPTSSPLLAW
WEWDTKFLCPLCMRSRRPRLETILALLVALQRLPVRLPEGEALQCLTERAISWQGRARQALASEDVTALLGRL
AELRQRLQAEPRPEEPPNYPAAPASDPLREGSGKDMPKVQGLLENGDSVTSPEKVAPEEGSDLELLSSLLPQL
TGPVLELPEATRAPLEELMMEGDLLEVTLDENHSIPESLDFCILTPRYCSDLSSWGPAPGVFPPW*
```

An amino acid sequence for the protein encoded by the KRTAP10-9 gene that is a regulator of T cell exhaustion/dysfunction resistance is available from the UniPROT database at accession no. P60411, cDNA NM_198690, with gene location at Chr 21: 44627093-44628378.

(SEQ ID NO: 160)
ACACACTCACAAACTCACTCACTGACACACTCACACACTCACTTACACCTCCCCCAGCTCACCTCCTCCCCAC
CCCAGCATGGCCGCGTCCACCATGTCCATCCGCTCCAGCGCTTACTCCGACTCCTGGCAGGTGGACGACTGCC
CAGAGAGCTGCTGTGAGCCCCCCTGCTGCGCCACCAGCTGCTGCGCCCCGGCCCCCTGCCTGACCCTGGTCTG
CACCCCAGTGAGCCGTGTATCCAGCCCCTGCTGCCAGGTGACCTGTGAGCCCAGCCCCTGCCAATCAGGCTGC
ACCAGCTCCTGCACGCCCTCGTGCTGCCAGCAGTCTAGCTGCCAGCCGGCTTACTGCACCTCCTCCCCCTGCC
AGCAGGCCTGCTGCGTGCCCGTCTGCTGCAAGCCTGTGTGCTGCGTGCCCGTCTGCTGTGGGGCTTCTTCGTG
CTGCCAACAGTCTAGCTACCAGCCAGCTTGCTGTGCCTCTTCCTCCTGCCAGCCGGCCTGCTGTGTGCCCGTC
TGCTGCAAACCTGTGTGCTGTGCGCCCACCTGCTCTGAGGATTCCTATTCATGCTGCCAACAGTCTAGCTGCC
AGCCAGCTTGCTGCACTTCCTCCCCCTGCCAGCAGTCCTACTGTGTGCCTGTCTGCTGTAAGCCTGTCTGCTG
CAAACCCATCTGCTGTGTGCCTGTCTGCTCTGGGGCTTCCTCTTTGTGCTGCCAGCAGTCTGGCTGCCAGCCG
GCTTGCTGCACCACCTCCTGCTGCAGACCCTCCTCCTCTGTGTCCCTCCTCTGCCGCCCTGTGTGCAGGCCCG
CCTGCTGCGTGCCCGTCTCCTCCTGCTGTGCCCCCACCTCCTCCCGCCAGCCCAGCTATTGCCGCCAGGCCTC
CTGTGTGTCCCTTCTCTGCCGCCCTGTGTGCTCCCGCCCGGCCTGCTACAGCTTCTCCTCAGGCCAGAAGTCC
AGCTGCTGACGGTCATGTCCCCCAGGGCCAGCCGGGCTCAGGCCCCACCTCCCTGCCAGTCCTTGGACCTCCC
AGCCCACCCAGCCTCAGCACAGCTCAACACAGAAGGAGCAGCCCCAGCCACAGCCGCCCAGCCCCGGGGTCTC
AGATGCTCACTGGCTCCTCCCTGACCTCCCCCCCGGGCAGGCGAGCCCTGGCTTCTCCTCACGGTGCTTCCTG
GCTGCAGACCACAACCCTCCGCTGGTCGCTGGTTGGGGACGGGCCCTCCTGACCCGGGTCTCACCCCCAGCAG
CGTCACCCTCTGCCCCTGAGCACCAATAAAGCGTCTGCGGCCCCA
(SEQ ID NO: 161)
MAASTMSIRSSAYSDSWQVDDCPESCCEPPCCATSCCAPAPCLTLVCTPVSRVSSPCCQVTCEPSPCQSGCTS
SCTPSCCQQSSCQPAYCTSSPCQQACCVPVCCKPVCCVPVCCGASSCCQQSSYQPACCASSSCQPACCVPVCC
KPVCCAPTCSEDSYSCCQQSSCQPACCTSSPCQQSYCVPVCCKPVCCKPICCVPVCSGASSLCCQQSGCQPAC
CTTSCCRPSSSVSLLCRPVCRPACCVPVSSCCAPTSSRQPSYCRQASCVSLLCRPVCSRPACYSFSSGQKSSC*

An amino acid sequence for the protein encoded by the MADD gene that is a regulator of T cell exhaustion/dysfunction resistance is available from the UniPROT database at accession no. Q8WXG6, cDNA NM_001376574, with gene location at Chr 11: 47269161-47330031.

```
  (SEQ ID NO: 162)
AGTGCAGACGCGGAACTGGCGCCCGCTCGGAGCGGCGCCGCGCTGGGGAGCGACTGACGCCCCGCTGCCGGGG
GACGTCGGGCTGGGCCTGGCCAGTCCCCCAGAGCTTGGGAGACTTCGATTTTCAGAATTCCTCCTGGGAATGC
TGACTCCTTGCTTGGTGCCCTGATGCTTCTCTGAGATAAACTGATGAATTGGAACCATGGTGCAAAAGAAGAA
GTTCTGTCCTCGGTTACTTGACTATCTAGTGATCGTAGGGGCCAGGCACCCGAGCAGTGATAGCGTGGCCCAG
ACTCCTGAATTGCTACGGCGATACCCCTTGGAGGATCACACTGAGTTTCCCCTGCCCCCAGATGTAGTGTTCT
TCTGCCAGCCCGAGGGCTGCCTGAGCGTGCGGCAGCGGCGCATGAGCCTTCGGGATGATACCTCTTTTGTCTT
CACCCTCACTGACAAGGACACTGGAGTCACGCGATATGGCATCTGTGTTAACTTCTACCGCTCCTTCCAAAAG
CGAATCTCTAAGGAGAAGGGGGAAGGTGGGGCAGGGTCCCGTGGGAAGGAAGGAACCCATGCCACCTGTGCCT
CAGAAGAGGGTGGCACTGAGAGCTCAGAGAGTGGCTCATCCCTGCAGCCTCTCAGTGCTGACTCTACCCCTGA
TGTGAACCAGTCTCCTCGGGGCAAACGCCGGGCCAAGGCGGGGAGCCGCTCCCGCAACAGTACTCTCACGTCC
CTGTGCGTGCTCAGCCACTACCCTTTCTTCTCCACCTTCCGAGAGTGTTTGTATACTCTCAAGCGCCTGGTGG
ACTGCTGTAGTGAGCGCCTTCTGGGCAAGAAACTGGGCATCCCTCGAGGCGTACAAAGGGACACCATGTGGCG
GATCTTTACTGGATCGCTGCTGGTAGAGGAGAAGTCAAGTGCCCTTCTGCATGACCTTCGAGAGATTGAGGCC
TGGATCTATCGATTGCTGCGCTCCCCAGTACCCGTCTCTGGGCAGAAGCGAGTAGACATCGAGGTCCTACCCC
AAGAGCTCCAGCCAGCTCTGACCTTTGCTCTTCCAGACCCATCTCGATTCACCCTAGTGGATTTCCCACTGCA
CCTTCCCTTGGAACTTCTAGGTGTGGACGCCTGTCTCCAGGTGCTAACCTGCATTCTGTTAGAGCACAAGGTG
GTGCTACAGTCCCGAGACTACAATGCACTCTCCATGTCTGTGATGGCATTCGTGGCAATGATCTACCCACTGG
AGTATATGTTTCCTGTCATCCCGCTGCTACCCACCTGCATGGCATCAGCAGAGCAGCTGCTGTTGGCTCCAAC
CCCGTACATCATTGGGGTTCCTGCCAGCTTCTTCCTCTACAAACTGGACTTCAAAATGCCTGATGATGTATGG
CTAGTGGATCTGGACAGCAATAGGGTGATTGCCCCCACCAATGCAGAAGTGCTGCCTATCCTGCCAGAACCAG
AATCACTAGAGCTGAAAAAGCATTTAAAGCAGGCCTTGGCCAGCATGAGTCTCAACACCCAGCCCATCCTCAA
TCTGGAGAAATTTCATGAGGGCCAGGAGATCCCCCTTCTCTTGGGAAGGCCTTCTAATGACCTGCAGTCCACA
CCGTCCACTGAATTCAACCCACTCATCTATGGCAATGATGTGGATTCTGTGGATGTTGCAACCAGGGTTGCCA
TGGTACGGTTCTTCAATTCCGCCAACGTGCTGCAGGGATTTCAGATGCACACGCGTACCCTGCGCCTCTTTCC
TCGGCCTGTGGTAGCTTTTCAAGCTGGCTCCTTTCTAGCCTCACGTCCCCGGCAGACTCCTTTTGCCGAGAAA
TTGGCCAGGACTCAGGCTGTGGAGTACTTTGGGGAATGGATCCTTAACCCCACCAACTATGCCTTTCAGCGAA
TTCACAACAATATGTTTGATCCAGCCCTGATTGGTGACAAGCCAAAGTGGTATGCTCATCAGCTGCAGCCTAT
CCACTATCGCGTCTATGACAGCAATTCCCAGCTGGCTGAGGCCCTGAGTGTACCACCAGAGCGGGACTCTGAC
TCCGAACCTACTGATGATAGTGGCAGTGATAGTATGGATTATGACGATTCAAGCTCTTCTTACTCCTCCCTTG
GTGACTTTGTCAGTGAAATGATGAAATGTGACATTAATGGTGATACTCCCAATGTGGACCCTCTGACACATGC
AGCACTGGGGGATGCCAGCGAGGTGGAGATTGACGAGCTGCAGAATCAGAAGGAAGCAGAAGAGCCTGGCCCA
GACAGTGAGAACTCTCAGGAAAACCCCCCACTGCGCTCCAGCTCTAGCACCACAGCCAGCAGCAGCCCCAGCA
CTGTCATCCACGGAGCCAACTCTGAACCTGCTGACTCTACGGAGATGGATGATAAGGCAGCAGTAGGCGTCTC
CAAGCCCCTCCCTTCCGTGCCTCCCAGCATTGGCAAATCGAACGTGGACAGACGTCAGGCAGAAATTGGAGAG
GGGTCAGTGCGCCGGCGAATCTATGACAATCCATACTTCGAGCCCCAATATGGCTTTCCCCCTGAGGAAGATG
AGGATGAGCAGGGGGAAAGTTACACTCCCCGATTCAGCCAACATGTCAGTGGCAATCGGGCTCAAAAGCTGCT
GCGGCCCAACAGCTTGAGACTGGCAAGTGACTCAGATGCAGAGTCAGACTCTCGGGCAAGCTCTCCCAACTCC
ACCGTCTCCAACACCAGCACCGAGGGCTTCGGGGGCATCATGTCTTTTGCCAGCAGCCTCTATCGGAACCACA
GTACCAGCTTCAGTCTTTCAAACCTCACACTGCCCACCAAAGGTGCCCGAGAGAAGGCCACGCCCTTCCCCAG
TCTGAAAGTATTTGGGCTAAATACTCTAATGGAGATTGTTACTGAAGCCGGCCCCGGGAGTGGTGAAGGAAAC
AGGAGGGCGTTAGTGGATCAGAAGTCATCTGTCATTAAACACAGCCCAACAGTGAAAAGAGAACCTCCATCAC
CCCAGGGTCGATCCAGCAATTCTAGTGAGAACCAGCAGTTCCTGAAGGAGGTGGTGCACAGCGTGCTGGACGG
CCAGGGAGTTGGCTGGCTCAACATGAAAAAGGTGCGCCGGCTGCTGGAGAGCGAGCAGCTGCGAGTCTTTGTC
CTGAGCAAGCTGAACCGCATGGTGCAGTCAGAGGACGATGCCCGGCAGGACATCATCCCGGATGTGGAGATCA
GTCGGAAGGTGTACAAGGGAATGTTAGACCTCCTCAAGTGTACAGTCCTCAGCTTGGAGCAGTCCTATGCCCA
CGCGGGTCTGGGTGGCATGGCCAGCATCTTTGGGCTTTTGGAGATTGCCCAGACCCACTACTATAGTAAAGAA
CCAGACAAGCGGAAGAGAAGTCCAACAGAAAGTGTAAATACCCCAGTTGGCAAGGATCCTGGCCTAGCTGGGC
GGGGGGACCCAAAGGCTATGGCACAACTGAGAGTTCCACAACTGGGACCTCGGGCACCAAGTGCCACAGGAAA
GGGTCCTAAGGAACTGGACACCAGAAGTTTAAAGGAAGAAAATTTTATAGCATCTATTGAATTGTGGAACAAG
CACCAGGAAGTGAAAAAGCAAAAAGCTTTGGAAAAACAGAGGCCTGAAGTAATCAAACCTGTCTTTGACCTTG
GTGAGACAGAGGAGAAAAAGTCCCAGATCAGCGCAGACAGTGGTGTGAGCCTGACGTCTAGTTCCCAGAGGAC
TGATCAAGACTCTGTCATCGGCGTGAGTCCAGCTGTTATGATCCGCAGCTCAAGTCAGGATTCTGAAGTTAGC
ACCGTGGTGAGTAATAGCTCTGGAGAGACCCTTGGAGCTGACAGTGACTTGAGCAGCAATGCAGGTGATGGAC
CAGGTGGCGAGGGCAGTGTTCACCTGGCAAGCTCTCGGGGCACTTTGTCTGATAGTGAAATTGAGACCAACTC
TGCCACAAGCACCATCTTTGGTAAAGCCCACAGCTTGAAGCCAAGCATAAAGGAGAAGCTGGCAGGCAGCCCC
ATTCGTACTTCTGAAGATGTGAGCCAGCGAGTCTATCTCTATGAGGGACTCCTAGGAAGGGACAAAGGATCCA
TGTGGGACCAGTTAGAGGATGCAGCTATGGAGACCTTTTCTATAAGCAAAGAGCGTTCTACTTTATGGGACCA
AATGCAATTCTGGGAAGATGCCTTCTTAGATGCTGTGATGTTGGAGAGAGAAGGGATGGGTATGGACCAGGGT
CCCCAGGAAATGATCGACAGGTACCTGTCCCTTGGAGAACATGACCGGAAGCGCCTGGAAGATGATGAAGATC
GCTTGTTGGCCACACTTCTGCACAACCTCATCTCCTACATGCTGCTGATGAAGGTAAATAAGAATGACATCCG
CAAGAAGGTGAGGCGCCTAATGGGAAAGTCGCACATTGGGCTTGTGTACAGCCAGCAAATCAATGAGGTGCTT
GATCAGCTGGCGAACCTGAATGGACGCGATCTCTCTATCTGGTCCAGTGGCAGCCGGCACATGAAGAAGCAGA
CATTTGTGGTACATGCAGGGACAGATACAAACGGAGATATCTTTTTCATGGAGGTGTGCGATGACTGTGTGGT
GTTGCGTAGTAACATCGGAACAGTGTATGAGCGCTGGTGGTACGAGAAGCTCATCAACATGACCTACTGTCCC
AAGACGAAGGTGTTGTGCTTGTGGCGTAGAAATGGCTCTGAGACCCAGCTCAACAAGTTCTATACTAAAAAGT
GTCGGGAGCTGTACTACTGTGTGAAGGACAGCATGGAGCGCGCTGCCGCCCGACAGCAAAGCATCAAACCCGG
ACCTGAATTGGGTGGCGAGTTCCCTGTGCAGGACCTGAAGACTGGTGAGGGTGGCCTGCTGCAGGTGACCCTG
GAAGGGATCAACCTCAAATTCATGCACAATCAGGAGCGGAAGGTTTTCATAGAGCTGAATCACATTAAAAAGT
GCAATACAGTTCGAGGCGTCTTTGTCCTGGAGGAATTTGTTCCTGAAATTAAAGAAGTGGTGAGCCACAAGTA
CAAGACACCAATGGCCCACGAAATCTGCTACTCCGTATTATGTCTCTTCTCGTACGTGGCTGCAGTTCATAGC
AGTGAGGAAGATCTCAGAACCCCGCCCCGGCCTGTCTCTAGCTGATGGAGAGGGGCTACGCAGCTGCCCCAGC
CCAGGGCACGCCCCTGGCCCCTTGCTGTTCCCAAGTGCACGATGCTGCTGTGACTGAGGAGTGGATGATGCTC
GTGTGTCCTCTGCAAGCCCCCTGCTGTGGCTTGGTTGGTTACCGGTTATGTGTCCCTCTGAGTGTGTCTTGAG
CGTGTCCACCTTCTCCCTCTCCACTCCCAGAAGACCAAACTGCCTTCCCCTCAGGGCTCAAGAATGTGTACAG
TCTGTGGGGCCGGTGTGAACCCACTATTTTGTGTCCTTGAGACATTTGTGTTGTGGTTCCTTGTCCTTGTCCC
TGGCGTTATAACTGTCCACTGCAAGAGTCTGGCTCTCCCTTCTCTGTGACCCGGCATGACTGGGCGCCTGGAG
CAGTTTCACTCTGTGAGGAGTGAGGGAACCCTGGGGCTCACCCTCTCAGAGGAAGGGCACAGAGAGGAAGGGA
AGAATTGGGGGGCAGCCGGAGTGAGTGGCAGCCTCCCTGCTTCCTTCTGCATTCCCAAGCCGGCAGCTACTGC
CCAGGGCCCGCAGTGTTGGCTGCTGCCTGCCACAGCCTCTGTGACTGCAGTGGAGCGGCGAATTCCCTGTGGC
CTGCCATGCCTTCGGCATCAGAGGATGGAGTGGTCGAGGCTAGTGGAGTCCCAGGGACCGCTGGCTGCTCTGC
CTGAGCATCAGGGAGGGGGCAGGAAAGACCAAGCTGGGTTTGCACATCTGTCTGCAGGCTGTCTCTCCAGGCA
CGGGGTGTCAGGAGGGAGAGACAGCCTGGGTATGGGCAAGAAATGACTGTAAATATTTCAGCCCCACATTATT
TATAGAAAATGTACAGTTGTGTGAATGTGAAATAAATGTCCTCAACTCCC
  (SEQ ID NO: 163)
MVQKKKFCPRLLDYLVIVGARHPSSDSVAQTPELLRRYPLEDHTEFPLPPDVVFFCQPEGCLSVRQRRMSLRD
DTSFVFTLTDKDTGVTRYGICVNFYRSFQKRISKEKGEGGAGSRGKEGTHATCASEEGGTESSESGSSLQPLS
ADSTPDVNQSPRGKRRAKAGSRSRNSTLTSLCVLSHYPFFSTFRECLYTLKRLVDCCSERLLGKKLGIPRGVQ
RDTMWRIFTGSLLVEEKSSALLHDLREIEAWIYRLLRSPVPVSGQKRVDIEVLPQELQPALTFALPDPSRFTL
VDFPLHLPLELLGVDACLQVLTCILLEHKVVLQSRDYNALSMSVMAFVAMIYPLEYMFPVIPLLPTCMASAEQ
LLLAPTPYIIGVPASFFLYKLDFKMPDDVWLVDLDSNRVIAPTNAEVLPILPEPESLELKKHLKQALASMSLN
TQPILNLEKFHEGQEIPLLLGRPSNDLQSTPSTEFNPLIYGNDVDSVDVATRVAMVRFFNSANVLQGFQMHTR
TLRLFPRPVVAFQAGSFLASRPRQTPFAEKLARTQAVEYFGEWILNPTNYAFQRIHNNMFDPALIGDKPKWYA
HQLQPIHYRVYDSNSQLAEALSVPPERDSDSEPTDDSGSDSMDYDDSSSSYSSLGDFVSEMMKCDINGDTPNV
DPLTHAALGDASEVEIDELQNQKEAEEPGPDSENSQENPPLRSSSSTTASSSPSTVIHGANSEPADSTEMDDK
AAVGVSKPLPSVPPSIGKSNVDRRQAEIGEGSVRRRIYDNPYFEPQYGFPPEEDEDEQGESYTPRFSQHVSGN
RAQKLLRPNSLRLASDSDAESDSRASSPNSTVSNTSTEGFGGIMSFASSLYRNHSTSFSLSNLTLPTKGAREK
ATPFPSLKVFGLNTLMEIVTEAGPGSGEGNRRALVDQKSSVIKHSPTVKREPPSPQGRSSNSSENQQFLKEVV
HSVLDGQGVGWLNMKKVRRLLESEQLRVFVLSKLNRMVQSEDDARQDIIPDVEISRKVYKGMLDLLKCTVLSL
EQSYAHAGLGGMASIFGLLEIAQTHYYSKEPDKRKRSPTESVNTPVGKDPGLAGRGDPKAMAQLRVPQLGPRA
PSATGKGPKELDTRSLKEENFIASIELWNKHQEVKKQKALEKQRPEVIKPVFDLGETEEKKSQISADSGVSLT
SSSQRTDQDSVIGVSPAVMIRSSSQDSEVSTVVSNSSGETLGADSDLSSNAGDGPGGEGSVHLASSRGTLSDS
EIETNSATSTIFGKAHSLKPSIKEKLAGSPIRTSEDVSQRVYLYEGLLGRDKGSMWDQLEDAAMETFSISKER
STLWDQMQFWEDAFLDAVMLEREGMGMDQGPQEMIDRYLSLGEHDRKRLEDDEDRLLATLLHNLISYMLLMKV
NKNDIRKKVRRLMGKSHIGLVYSQQINEVLDQLANLNGRDLSIWSSGSRHMKKQTFVVHAGTDTNGDIFFMEV
CDDCVVLRSNIGTVYERWWYEKLINMTYCPKTKVLCLWRRNGSETQLNKFYTKKCRELYYCVKDSMERAAARQ
QSIKPGPELGGEFPVQDLKTGEGGLLQVTLEGINLKFMHNQVFIELNHIKKCNTVRGVFVLEEFVPEIKEVVS
HKYKTPMAHEICYSVLCLFSYVAAVHSSEEDLRTPPRPVSS*
```

An amino acid sequence for the protein encoded by the MAPKAP1 gene that is a regulator of T cell exhaustion/dysfunction resistance is available from the UniPROT database at accession no. Q9BPZ7, cDNA NM 001006617, with gene location at Chr 9: 125437393-125707234.

```
  (SEQ ID NO: 164)
GTCGTGTGCGGCTCGGGGTAATAGGGCTGCTGCTCGGCCGGCCGGCGGCGGCGAGCAGCAGGGGCATGAGGGC
TAACCCGGGAAGCGGCAGCTGAGCGGGCCGGGAGGAGCGCCGGTCCCCGTGGATCCCGAGAGTGCAGAGCTCG
GGGCAGGGGCCGGGAGGCGTGGGGGAGCCGGGCCCTCCCCTCAGGAACGTGTCCCGGGGCCGACCCGGCCCGT
AGTGTGGAAGCAGCTTCAGGTAGGTGAGCTCGTGAAACAATATGAAGAGGAGAAAATAGCCTTTTAAGGAAAT
TGGCCCACAGAAAGGATGGCCTTCTTGGACAATCCAACTATCATTCTAGCTCATATTCGACAGTCACATGTGA
CCAGTGATGACACGGGAATGTGTGAGATGGTTCTCATTGATCATGATGTTGACCTAGAGAAGATTCATCCTCC
TTCAATGCCTGGAGACAGTGGGTCAGAAATTCAGGGAAGCAATGGTGAGACTCAGGGCTATGTATATGCCCAG
TCAGTCGATATTACCTCAAGTTGGGACTTTGGTATTAGAAGACGCTCAAACACAGCTCAAAGATTAGAACGAC
TCCGAAAAGAGAGACAAAACCAGATCAAATGCAAAAATATTCAGTGGAAAGAAAGAAATTCTAAGCAATCAGC
CCAGGAGTTAAAGTCACTGTTTGAAAAAAAATCTCTCAAAGAGAAGCCTCCAATTTCTGGGAAGCAGTCGATA
TTATCTGTACGCCTAGAACAGTGCCCTCTGCAGCTGAATAACCCTTTTAACGAGTATTCCAAATTTGATGGCA
AGGGTCATGTAGGTACAACAGCAACCAAGAAGATCGATGTCTACCTCCCTCTGCACTCGAGCCAGGACAGACT
GCTGCCAATGACCGTGGTGACAATGGCCAGCGCCAGGGTGCAGGACCTGATCGGGCTCATCTGCTGGCAGTAT
ACAAGCGAAGGACGGGAGCCGAAGCTCAATGACAATGTCAGTGCCTACTGCCTGCATATTGCTGAGGATGATG
GGGAGGTGGACACCGATTTCCCCCCGCTGGATTCCAATGAGCCCATTCATAAGTTTGGCTTCAGTACTTTGGC
CCTGGTTGAAAAGTACTCATCTCCTGGTCTGACATCCAAAGAGTCACTCTTTGTTCGAATAAATGCTGCTCAT
GGATTCTCCCTTATTCAGGTGGACAACACAAAGGTTACCATGAAGGAAATCTTACTGAAGGCAGTGAAGCGAA
GAAAAGGATCCCAGAAAGTTTCAGGCCCTCAGTACCGCCTGGAGAAGCAGAGCGAGCCCAATGTCGCCGTTGA
CCTGGACAGCACTTTGGAGAGCCAGAGCGCATGGGAGTTCTGCCTGGTCCGCGAGAACAGTTCAAGGGCAGAC
GGGGTTTTTGAGGAGGATTCGCAAATTGACATAGCCACAGTACAGGATATGCTTAGCAGCCACCATTACAAGT
CATTCAAAGTCAGCATGATCCACAGACTGCGATTCACAACCGACGTACAGCTAGGTATCTCTGGAGACAAAGT
AGAGATAGACCCTGTTACGAATCAGAAAGCCAGCACTAAGTTTTGGATTAAGCAGAAACCCATCTCAATCGAT
TCCGACCTGCTCTGTGCCTGTGACCTTGCTGAAGAGAAAAGCCCCAGTCACGCAATATTTAAACTCACGTATC
TAAGCAATCACGACTATAAACACCTCTACTTTGAATCGGACGCTGCTACCGTCAATGAAATTGTGCTCAAGGT
TAACTACATCCTGGAATCGCGAGCTAGCACTGCCCGGGCTGACTACTTTGCTCAAAAACAAAGAAAACTGAAC
AGACGTACGAGCTTCAGCTTCCAGAAGGAGAAGAAATCCGGGCAGCAGTGACACTGGCCTCCAGCCTCAATCT
GTTCCGTAGCTCAGAGCCTGCCTGCCAGGGCCAAGTGCCCTAGAGTCCACCCGGTGTCCTGAAGTCCTCGGGG
GGAGGCCAGCCCCTGGCTCACTGGCACAGGGCAGGTGGGCTCTCGGGGAAGGTGTCGGGGGCCCCCTAGGAGG
GAGCGCTGGGGACATTGCCATGGGACGGAAGTCTGCTTGGCAGTGGCTTTGATAAGCGATGCTTGGGGGTCAG
ACCACCCCCTAGAGGAGCCACGTGCCGCCCAGCCACCTTCAATGCCTGCCACCCTGCCCGAGGATGTACAGAG
CCGTGCCCACACATTTCCTTGCAACTTGATCAAATTTCTTAAAGCAAACAACAAAAATGTACATTTCTGTTTT
TCCTTTTAATAAACAGGTGTACTCTTTATCATGGTTGGTATGATGGACCATTCTTTGGGGCGGAGGATTGATT
ATGTTACTCTCTTTAAAATCTGTTCCCATATTGAACAGGCAGATTGGAAAAGCTATGGTTCGATTTCTCAGAA
GAAATGTTTAGGTCTTAGTCAATAGTTTTAACTATGCCATTTGTTTAAATGAGTGCATTTGCTTCGAGGGTAG
TGTCTTACTAAAAGTTAGGAACAGAGACCTAGTGGTGTGTCCAAGGCCGTGTCACTTTCCCCTTCAGCACACC
CCAGCTTCTGACCTCAGAGCCCAGGAGCTGCGTGGACAGTGTGGGGTGCCAGGAGGAGGGGCGGTGGCTGGTC
CTCAGGCACGCTGCACTCCCAGCCAGACATGGTCTTTCCGTTTCTTAAGTAGCAAGTGTAGGTTTCAGCTGGC
AGTTCCACCTGCATGTTCTCTGCTTCGCTGCCTTGGAAGGGGCCACATTCCCCATTCCTCTTCTCCTTACAGC
GCCTGCCTCCTTTTTCAAGCAGGCGGAAAGCTGCTGTTTCTCACGTTTCAGGGAGAGGGGTGAGCGGAGGGAG
ACCTGTGTCCGTGCCGTCCGGCTCCCTGGGTGGGAACAGGCAAGGGATCAGATGCCCCTGACACCACGCCTCT
GGCCACACCAGATGCCTCTGCAGTCCTCGACAGCCTCTTCAGTGTCCCTCCTGCGGTGATGTCCTTACTGTCC
CCAGCCAGGGCCGGGGACCGGTGTTTCACTGAGGACCTGCATTAGAAACATTTTTTAAATTGTTGTACAGGAA
GAGATGTGTCTAAAACAGCATCTTAAAGCTGAGTGTATTTCTTTGCACAAGGGGTCATGCTGATGAATTCTTC
TTTCATTCTGATCTTTGTTCAGCCAACAGGAGCGTCCTTTTCTAATGTCTTCCATTCCTACCCCCCACCCAAA
AACAAAAGAAATATTTGTAGCTTGCTATCTGTATTTGAATTTTTAGCAATTTTATATTTAGATACTTTGAAAA
ATGTAAATGACTAATTTGGTCATTAAATCTTGTGACATATTCGATATTAAAATGATATTAAAATAAAAGTCAT
ATAAATACACA
  (SEQ ID NO: 165)
MAFLDNPTIILAHIRQSHVTSDDTGMCEMVLIDHDVDLEKIHPPSMPGDSGSEIQGSNGETQGYVYAQSVDIT
SSWDFGIRRRSNTAQRLERLRKERQNQIKCKNIQWKERNSKQSAQELKSLFEKKSLKEKPPISGKQSILSVRL
EQCPLQLNNPFNEYSKFDGKGHVGTTATKKIDVYLPLHSSQDRLLPMTVVTMASARVQDLIGLICWQYTSEGR
EPKLNDNVSAYCLHIAEDDGEVDTDFPPLDSNEPIHKFGFSTLALVEKYSSPGLTSKESLFVRINAAHGFSLI
QVDNTKVTMKEILLKAVKRRKGSQKVSGPQYRLEKQSEPNVAVDLDSTLESQSAWEFCLVRENSSRADGVFEE
DSQIDIATVQDMLSSHHYKSFKVSMIHRLRFTTDVQLGISGDKVEIDPVTNQKASTKFWIKQKPISIDSDLLC
ACDLAEEKSPSHAIFKLTYLSNHDYKHLYFESDAATVNEIVLKVNYILESRASTARADYFAQKQRKLNRRTSF
SFQKEKKSGQQ*
```

An amino acid sequence for the protein encoded by the MED23 gene that is a regulator of T cell exhaustion/dysfunction resistance is available from the UniPROT database at accession no. Q9ULK4, cDNA NM_001270521, with gene location at Chr 6: 131573966-131628242.

(SEQ ID NO: 166)
GAGGGAACGCTCCAGGTAAAGCGCTGGGGGAGGAAGCGACGCCGAGGAGCTACGGTTTCCTCCAGAGGTCTCC
GCCCCTCTGCCCCTATATTCCCAGAGCTCGAGTCTGATCCGGGCCTTGCCGGGCACCCTGGAAAGGCGGGGGT
GATAGTACAGATGGAGACGCAACTGCAGAGCATTTTCGAAGAGGTGGTGAAAACGGAAGTTATAGAAGAGGCT
TTTCCTGGCATGTTTATGGATACTCCTGAAGATGAGAAAACAAAACTAATTAGCTGTTTGGGGGCCTTCAGAC
AGTTTTGGGGTGGTCTTTCTCAGGAGTCTCATGAACAGTGTATCCAGTGGATTGTTAAGTTTATTCATGGTCA
GCATAGTCCTAAAAGAATTTCTTTTCTTTATGACTGCTTAGCAATGGCAGTTGAGACTGGTCTCCTTCCACCC
AGGCTGGTTTGTGAATCCCTGATAAACTCTGACACTCTTGAGTGGGAAAGAACACAGCTTTGGGCCTTAACAT
TTAAACTGGTTCGGAAAATAATTGGGGGAGTGGATTACAAGGGTGTTCGAGATCTCTTAAAAGTGATTTTGGA
GAAGATTTTGACAATTCCTAATACAGTGAGCTCTGCTGTTGTACAGCAGCTTCTGGCAGCAAGAGAGGTTATA
GCATATATCTTGGAAAGAAATGCCTGCTTATTACCAGCCTATTTTGCAGTCACTGAGATCAGGAAACTGTATC
CTGAAGGCAAACTTCCACACTGGTTACTTGGAAACCTAGTATCAGACTTTGTGGATACCTTCAGGCCCACAGC
AAGGATAAACTCCATTTGTGGTCGCTGTAGTCTTCTGCCAGTTGTAAATAATTCGGGTGCCATTTGTAATTCA
TGGAAACTGGATCCTGCTACTCTTCGTTTTCCTTTGAAAGGCCTTTTGCCATATGATAAGGATCTGTTTGAAC
CACAGACTGCTTTGTTGAGATATGTATTGGAGCAGCCTTATTCCAGGGATATGGTCTGCAATATGCTAGGTTT
AAATAAGCAGACCTTGAACATTGCTCAGCACAAGCAGCGCTGCCCTGTGCTGGAGGACCAGTTGGTGGATCTG
GTTGTTTATGCCATGGAGCGATCTGAGACCGAGGAGAAGTTTGACGATGGGGGAACAAGCCAACTCCTGTGGC
AGCATCTCTCAAGTCAGCTCATTTTCTTTGTGCTTTTCCAGTTTGCAAGTTTTCCACATATGGTGCTTTCTCT
TCATCAGAAGTTAGCAGGGCGAGGACTGATTAAAGGCAGAGATCATCTTATGTGGGTTCTCCTGCAATTCATT
TCTGGAAGTATTCAGAAAAATGCACTAGCTGATTTTCTCCCTGTGATGAAGCTCTTCGACTTGCTATACCCAG
AAAAAGAATATATCCCAGTTCCTGATATTAACAAACCCCAGTCAACCCATGCCTTTGCAATGACCTGTATTTG
GATTCATCTCAATAGAAAAGCTCAAAATGACAACTCCAAGCTACAGATTCCAATACCTCATTCCCTAAGACTT
CACCATGAGTTCCTGCAGCAGAGTCTAAGAAATAAAAGTTTACAGATGAATGACTATAAGATTGCTCTATTGT
GTAATGCATACTCTACAAATTCAGAATGTTTTACATTACCCATGGGAGCTCTGGTAGAAACTATTTATGGAAA
TGGAATTATGAGGATACCTCTCCCTGGAACAAACTGTATGGCTTCAGGATCTATTACCCCCTTACCTATGAAC
CTCCTGGATTCACTGACAGTTCATGCCAAAATGAGCCTTATTCACAGCATTGCAACCAGGGTGATAAAACTTG
CTCATGCAAAGTCCAGTGTGGCCTTGGCTCCAGCCCTAGTGGAAACTTACAGTCGTTTATTGGTCTATATGGA
AATAGAGTCTTTGGGCATCAAAGGATTTATCAGTCAGCTTTTGCCAACTGTTTTCAAATCACATGCATGGGGG
ATCTTACACACACTCCTTGAGATGTTTAGCTACCGGATGCATCATATTCAGCCTCATTACAGAGTTCAGCTCC
TGAGTCATCTTCATACTTTGGCTGCAGTTGCACAAACAAACCAGAACCAGCTCCATCTTTGTGTCGAGAGCAC
TGCTCTCAGGCTTATAACAGCATTAGGTAGCTCAGAGGTACAACCGCAGTTTACACGCTTCCTTAGTGATCCC
AAAACAGTGCTCTCAGCAGAATCTGAAGAACTGAACCGAGCCTTGATATTGACCTTGGCTAGAGCAACTCATG
TAACAGATTTTTTTACAGGCTCTGATTCAATTCAGGGAACTTGGTGTAAAGACATACTTCAGACCATCATGAG
TTTCACTCCTCATAATTGGGCTTCACACACCCTGAGCTGTTTTCCAGGCCCACTACAGGCATTCTTCAAACAA
AATAATGTGCCTCAGGAAAGCCGTTTTAATCTGAAAAAAAATGTGGAGGAGGAGTATAGGAAGTGGAAGTCAA
TGAGCAACGAAAACGACATTATTACCCACTTCTCTATGCAGGGCTCCCCTCCTCTCTTTCTTTGTCTTCTCTG
GAAAATGCTCTTGGAAACAGATCATATTAATCAGATTGGCTATAGAGTATTAGAGAGAATTGGAGCCAGGGCC
TTGGTAGCCCATGTGAGGACATTTGCAGATTTCCTGGTATATGAGTTTTCTACATCAGCAGGGGGTCAGCAAC
TCAATAAATGCATTGAAATTCTTAATGACATGGTATGGAAGTATAACATTGTTACACTGGACAGATTAATTCT
CTGCCTGGCCATGCGTAGTCACGAAGGAAATGAAGCCCAGGTTTGTTATTTCATAATTCAGTTGCTGTTACTC
AAACCAAACGATTTTAGAAATCGAGTAAGTGACTTTGTGAAGGAAAATTCCCCAGAGCACTGGTTACAGAATG
ACTGGCACACCAAGCACATGAATTATCACAAGAAATATCCAGAGAAGTTGTATTTTGAGGGCCTCGCGGAACA
GGTGGATCCTCCTGTACAGATCCAGTCTCCCTATCTGCCCATCTATTTTGGGAATGTGTGTCTTCGATTCCTT
CCAGTATTTGATATAGTAATCCACAGATTTTTAGAGTTGCTTCCGGTATCCAAATCACTGGAGACTCTACTGG
ATCATCTAGGAGGCTTATATAAATTTCATGATCGTCCAGTGACTTATCTGTATAACACTCTGCACTATTATGA
AATGCACCTGAGAGACCGCGCATTTCTCAAACGAAAACTCGTCCATGCGATCATTGGCTCTCTGAAGGATAAT
CGACCGCAGGGCTGGTGTCTAAGTGACACTTACCTGAAATGCGCTATGAATGCACGAGAGGAAAATCCTTGGG
TTCCAGATGACACCTACTATTGCAGATTGATTGGCAGACTAGTCGATACGATGGCTGGCAAATCTCCTGGTCC
CTTTCCAAACTGTGACTGGAGATTCAATGAGTTTCCCAACCCAGCTGCCCATGCTCTCCATGTTACTTGTGTG
GAGCTCATGGCCTTGGCAGTTTCAGGCAAAGAAGTTGGGAATGCCCTTCTAAATGTTGTCCTAAAAAGTCAGC
CTTTAGTGCCAAGAGAGAACATTACAGCATGGATGAATGCAATTGGTTTGATCATCACTGCCCTACCAGAGCC
ATATTGGATTGTTCTTCATGATCGAATTGTGAGTGTCATCAGCAGCCCCAGCTTGACGTCTGAAACAGAGTGG
GTTGGCTATCCATTCCGCCTCTTTGATTTCACTGCCTGTCATCAGTCCTACTCTGAGATGAGTTGTAGCTATA
CGTTAGCTCTTGCACATGCTGTGTGGCACCATTCTAGCATCGGACAACTTTCTCTCATTCCAAAGTTTCTTAC
TGAAGTACTTCTTCCTATAGTGAAGACCGAATTCCAGTTGCTTTATGTATACCATCTTGTTGGACCATTTTTA
CAAAGATTTCAGCAAGAGAGAACTCGTTGTATGATAGAGATTGGTGTGGCGTTTTATGACATGCTGCTGAATG
TTGACCAGTGTAGCACCCATTTAAATTACATGGATCCCATCTGTGACTTCCTCTATCACATGAAGTATATGTT
TACTGGTGACAGCGTGAAAGAGCAAGTAGAGAAGATTATCTGTAACTTAAAACCAGCTTTAAAACTTCGTCTT
CGATTCATCACACACATTAGCAAGATGGAGCCAGCTGCAGTGCCTCCACAAGCCATGAACAGTGGGTCTCCAG
CACCTCAGTCTAATCAGTAAAAAGTCAGAGTACACAGCAAGGGTTCCAAATTAAGAGGAAGCAAAACGCTGCC
CAGATCCTGAGAAACCTGGAGAAAATCAACCTTAGTAGTTTCAACTCATACATTGTCTTGTTTGATTTTGTTC
TTTGTTTTTTATCCTCTTTAAGCACAGATGCGTGTTGAAGTCTAAGGTGTTCTGTCATCACACTCTCTTTACT
AGAATAAGTGTGCCCAGGCAGAAAAAAAAGTGTATTCAATGTTGCTGGCATGTGTGGATTCACAGCTCGTGGC
TGTAAGGAAATCTGGGGGTTTCATAAATCCATTAAAATCCTTTTAGAGTTGCCAA
(SEQ ID NO: 167)
METQLQSIFEEVVKTEVIEEAFPGMFMDTPEDEKTKLISCLGAFRQFWGGLSQESHEQCIQWIVKFIHGQHSP
KRISFLYDCLAMAVETGLLPPRLVCESLINSDTLEWERTQLWALTFKLVRKIIGGVDYKGVRDLLKVILEKIL
TIPNTVSSAVVQQLLAAREVIAYILERNACLLPAYFAVTEIRKLYPEGKLPHWLLGNLVSDFVDTFRPTARIN
SICGRCSLLPVVNNSGAICNSWKLDPATLRFPLKGLLPYDKDLFEPQTALLRYVLEQPYSRDMVCNMLGLNKQ
TLNIAQHKQRCPVLEDQLVDLVVYAMERSETEEKFDDGGTSQLLWQHLSSQLIFFVLFQFASFPHMVLSLHQK
LAGRGLIKGRDHLMWVLLQFISGSIQKNALADFLPVMKLEDLLYPEKEYIPVPDINKPQSTHAFAMTCIWIHL
NRKAQNDNSKLQIPIPHSLRLHHEFLQQSLRNKSLQMNDYKIALLCNAYSTNSECFTLPMGALVETIYGNGIM
RIPLPGTNCMASGSITPLPMNLLDSLTVHAKMSLIHSIATRVIKLAHAKSSVALAPALVETYSRLLVYMEIES
LGIKGFISQLLPTVFKSHAWGILHTLLEMFSYRMHHIQPHYRVQLLSHLHTLAAVAQTNQNQLHLCVESTALR
LITALGSSEVQPQFTRFLSDPKTVLSAESEELNRALILTLARATHVTDFFTGSDSIQGTWCKDILQTIMSFTP
HNWASHTLSCFPGPLQAFFKQNNVPQESRFNLKKNVEEEYRKWKSMSNENDIITHFSMQGSPPLFLCLLWKML
LETDHINQIGYRVLERIGARALVAHVRTFADFLVYEFSTSAGGQQLNKCIEILNDMVWKYNIVTLDRLILCLA
MRSHEGNEAQVCYFIIQLLLLKPNDERNRVSDFVKENSPEHWLQNDWHTKHMNYHKKYPEKLYFEGLAEQVDP
PVQIQSPYLPIYFGNVCLRFLPVFDIVIHRELELLPVSKSLETLLDHLGGLYKFHDRPVTYLYNTLHYYEMHL
RDRAFLKRKLVHAIIGSLKDNRPQGWCLSDTYLKCAMNAREENPWVPDDTYYCRLIGRLVDTMAGKSPGPFPN
CDWRENEFPNPAAHALHVTCVELMALAVSGKEVGNALLNVVLKSQPLVPRENITAWMNAIGLIITALPEPYWI
VLHDRIVSVISSPSLTSETEWVGYPFRLFDFTACHQSYSEMSCSYTLALAHAVWHHSSIGQLSLIPKELTEVL
LPIVKTEFQLLYVYHLVGPFLQRFQQERTRCMIEIGVAFYDMLLNVDQCSTHLNYMDPICDFLYHMKYMFTGD
SVKEQVEKIICNLKPALKLRLRFITHISKMEPAAVPPQAMNSGSPAPQSNQ*

An amino acid sequence for the protein encoded by the MED24 gene that is a regulator of T cell exhaustion/dysfunction resistance is available from the UniPROT database at accession no. 075448, cDNA NM_001079518, with gene location at Chr 17: 40019097-40061215.

```
  (SEQ ID NO: 168)
AAGTGCCAAAATGGCGATGCCTACCACCTAGAACTGGATTGTGCGCTCCATTTCTTTCACCTCAAAGCTGGCC
GCCACCGCTGCCACCTGCTCAGAGTGAAATAATGAAGGTGGTCAACCTGAAGCAAGCCATTTTGCAAGCCTGG
AAGGAGCGCTGGAGTGACTACCAATGGGCAATCAACATGAAGAAATTCTTTCCTAAAGGAGCCACCTGGGATA
TTCTCAACCTGGCAGATGCGTTACTAGAGCAGGCCATGATTGGACCATCCCCCAATCCTCTCATCTTGTCCTA
CCTGAAGTATGCCATTAGTTCCCAGTTTGATGACTTTTCTCGGGACCTGTGTGTCCAGGCATTGCTGGACATC
ATGGACATGTTTTGTGACCGTCTGAGCTGTCACGGCAAAGCAGAGGAATGCATCGGACTGTGCCGAGCCCTTC
TTAGCGCCCTCCACTGGCTGCTGCGCTGCACGGCAGCCTCTGCAGAGCGGCTGCGGGAGGGGCTGGAGGCCGG
CACTCCAGCCGCTGGGGAGAAGCAGCTTGCCATGTGCCTTCAGCGCCTGGAGAAAACCCTCAGCAGCACCAAG
AACCGGGCCCTGCTGCACATCGCCAAACTAGAGGAGGCCTCTTCTTGGACTGCCATCGAGCATTCTCTCTTGA
AACTTGGAGAGATCCTGGCCAATCTCAGCAACCCGCAGCTCCGGAGTCAGGCCGAGCAGTGTGGCACCCTCAT
TAGGAGCATCCCCACGATGCTGTCTGTGCATGCGGAGCAGATGCACAAGACCGGCTTCCCCACTGTCCACGCC
GTGATCCTGCTCGAGGGCACCATGAACCTGACAGGCGAGACGCAGTCCCTGGTGGAGCAGCTGACGATGGTGA
AGCGCATGCAGCATATCCCCACCCCACTTTTTGTCCTGGAGATCTGGAAAGCTTGCTTCGTGGGGCTCATTGA
GTCTCCCGAGGGTACGGAGGAGCTCAAGTGGACAGCTTTCACTTTCCTCAAGATTCCACAGGTTTTGGTGAAG
TTGAAGAAGTACTCTCATGGAGACAAGGACTTCACTGAGGATGTCAACTGTGCTTTTGAGTTCCTGCTGAAGC
TCACCCCCTTGTTGGACAAAGCTGACCAGCGCTGCAACTGTGACTGTACAAACTTCCTGCTCCAAGAATGTGG
CAAGCAGGGGCTTCTGTCTGAGGCCAGCGTCAACAACCTTATGGCTAAGCGCAAAGCGGACCGAGAGCACGCA
CCCCAGCAGAAATCGGGAGAGAATGCCAACATCCAGCCCAACATCCAGCTGATCCTCCGGGCGGAGCCCACTG
TCACAAACATCCTCAAGACGATGGATGCAGACCACTCTAAGTCACCGGAGGGACTGCTGGGAGTCCTGGGCCA
CATGCTGTCCGGGAAGAGTCTGGACTTGCTGCTGGCTGCCGCCGCCGCCACTGGAAAGCTGAAATCCTTCGCC
CGGAAATTCATCAATTTGAATGAATTCACAACCTATGGCAGCGAAGAAAGCACCAAACCGGCCTCCGTCCGGG
CCCTGCTGTTTGACATCTCCTTCCTCATGCTGTGCCATGTGGCCCAGACCTATGGTTCAGAGGTGATTCTGTC
CGAGTCGCGCACAGGAGCTGAGGTGCCCTTCTTCGAGACCTGGATGCAGACCTGCATGCCTGAGGAGGGCAAG
ATCCTGAACCCTGACCACCCCTGCTTCCGCCCCGACTCCACCAAAGTGGAGTCCCTGGTGGCCCTGCTCAACA
ACTCCTCGGAGATGAAGCTAGTGCAGATGAAGTGGCATGAGGCCTGTCTCAGCATCTCAGCCGCCATCTTGGA
AATCCTCAATGCCTGGGAGAATGGGGTCCTGGCCTTCGAGTCCATCCAGAAAATCACTGATAACATCAAAGGG
AAGGTATGCAGTCTGGCGGTGTGTGCTGTGGCTTGGCTTGTGGCCCACGTCCGGATGCTGGGGCTGGATGAGC
GTGAGAAGTCGCTGCAGATGATCCGCCAGCTGGCAGGGCCACTGTTTAGTGAGAACACCCTGCAGTTCTACAA
TGAGAGGGTGGTGATCATGAACTCGATCCTGGAGCGCATGTGTGCCGACGTGCTGCAGCAGACAGCCACGCAG
ATCAAGTTTCCCTCCACCGGGGTGGACACAATGCCCTACTGGAACCTGCTGCCCCCCAAGCGGCCCATCAAAG
AGGTGCTGACGGACATTTTTGCCAAGGTGCTGGAGAAGGGCTGGGTGGACAGCCGCTCCATCCACATCTTTGA
CACCCTGCTGCACATGGGCGGCGTCTACTGGTTCTGCAACAACCTGATTAAGGAGCTGCTGAAGGAGACGCGG
AAGGAGCACACGCTGCGGGCAGTGGAGCTGCTCTACTCCATCTTCTGCCTGGACATGCAGCAAGTGACCCTGG
TCCTGCTGGGCCACATCCTACCTGGCCTGCTCACTGACTCCTCCAAGTGGCACAGCCTCATGGACCCCCCGGG
CACTGCTCTTGCCAAGCTGGCCGTGTGGTGTGCCCTCAGTTCCTACTCCTCCCACAAGGGACAGGCGTCCACC
CGCCAGAAGAAGAGACACCGCGAAGACATTGAGGATTATATCAGCCTCTTCCCCCTGGACGATGTGCAGCCTT
CGAAGTTGATGCGACTGCTGAGCTCTAATGAGGACGATGCCAACATCCTTTCGAGCCCCACAGACCGATCCAT
GAGCAGCTCCCTCTCAGCCTCTCAGCTCCACACGGTCAACATGCGGGACCCTCTGAACCGAGTCCTGGCCAAC
CTGTTCCTGCTCATCTCCTCCATCCTGGGGTCTCGCACCGCTGGCCCCCACACCCAGTTCGTGCAGTGGTTCA
TGGAGGAGTGTGTGGACTGCCTGGAGCAGGGTGGCCGTGGCAGCGTCCTGCAGTTCATGCCCTTCACCACCGT
GTCGGAACTGGTGAAGGTGTCAGCCATGTCCAGCCCCAAGGTGGTTCTGGCCATCACGGACCTCAGCCTGCCC
CTGGGCCGCCAGGTGGCTGCTAAAGCCATTGCTGCACTCTGAGGGGCTTGGCATGGCCGCAGTGGGGGCTGGG
GACTGGCGCAGCCCCAGGCGCCTCCAAGGGAAGCAGTGAGGAAAGATGAGGCATCGTGCCTCACATCCGCTCC
ACATGGTGCAAGAGCCTCTAGCGGCTTCCAGTTCCCCGCTCCTGACTCCTGACCTCCAGGATGTCTCCCGGTT
TCTTCTTTCAAAATTTCCTCTCCATCTGCTGGCACCTGAGGAGAGTGAGCAGCCTGGACCACAAGCCCAGTGG
TCACCCCTGTGTGCGCCCGCCCCAGCCCAGGAGTAGTCTTACCTCTGAGGAACTTTCTAGATGCAAAGTGTGT
ATGTGTGTGTGTGTGTGTGTGTGTGTGTGTTTGTGTGTATTTTGTAATATGTGAGGGAAATCTACCTTCGTTC
ATGTATAAATAAAGCTCCTCGTGGC
  (SEQ ID NO: 169)
MKVVNLKQAILQAWKERWSDYQWAINMKKFFPKGATWDILNLADALLEQAMIGPSPNPLILSYLKYAISSQFD
DFSRDLCVQALLDIMDMFCDRLSCHGKAEECIGLCRALLSALHWLLRCTAASAERLREGLEAGTPAAGEKOLA
MCLQRLEKTLSSTKNRALLHIAKLEEASSWTAIEHSLLKLGEILANLSNPQLRSQAEQCGTLIRSIPTMLSVH
AEQMHKTGFPTVHAVILLEGTMNLTGETQSLVEQLTMVKRMQHIPTPLFVLEIWKACFVGLIESPEGTEELKW
TAFTFLKIPQVLVKLKKYSHGDKDFTEDVNCAFEFLLKLTPLLDKADQRCNCDCTNELLQECGKQGLLSEASV
NNLMAKRKADREHAPQQKSGENANIQPNIQLILRAEPTVTNILKTMDADHSKSPEGLLGVLGHMLSGKSLDLL
LAAAAATGKLKSFARKFINLNEFTTYGSEESTKPASVRALLFDISFLMLCHVAQTYGSEVILSESRTGAEVPF
FETWMQTCMPEEGKILNPDHPCFRPDSTKVESLVALLNNSSEMKLVQMKWHEACLSISAAILEILNAWENGVL
AFESIQKITDNIKGKVCSLAVCAVAWLVAHVRMLGLDEREKSLQMIRQLAGPLFSENTLQFYNERVVIMNSIL
ERMCADVLQQTATQIKFPSTGVDTMPYWNLLPPKRPIKEVLTDIFAKVLEKGWVDSRSIHIFDTLLHMGGVYW
FCNNLIKELLKETRKEHTLRAVELLYSIFCLDMQQVTLVLLGHILPGLLTDSSKWHSLMDPPGTALAKLAVWC
ALSSYSSHKGQASTRQKKRHREDIEDYISLFPLDDVQPSKLMRLLSSNEDDANILSSPTDRSMSSSLSASQLH
TVNMRDPLNRVLANLFLLISSILGSRTAGPHTQFVQWEMEECVDCLEQGGRGSVLQFMPFTTVSELVKVSAMS
SPKVVLAITDLSLPLGRQVAAKAIAAL*
```

An amino acid sequence for the protein encoded by the MYB gene that is a regulator of T cell exhaustion/dysfunction resistance is available from the UniPROT database at accession no. P10242, cDNA NM_001130172, with gene location at Chr 6: 135181308-135219173.

```
  (SEQ ID NO: 170)
ACACTTTAATATCAACCTGTTTCCTCCTCCTCCTTCTCCTCCTCCTCCGTGACCTCCTCCTCCTCTTTCTCCT
GAGAAACTTCGCCCCAGCGGTGCGGAGCGCCGCTGCGCAGCCGGGGAGGGACGCAGGCAGGCGGCGGGCAGCG
GGAGGCGGCAGCCCGGTGCGGTCCCCGCGGCTCTCGGCGGAGCCCCGCGCCCGCCGCGCCATGGCCCGAAGAC
CCCGGCACAGCATATATAGCAGTGACGAGGATGATGAGGACTTTGAGATGTGTGACCATGACTATGATGGGCT
GCTTCCCAAGTCTGGAAAGCGTCACTTGGGGAAAACAAGGTGGACCCGGGAAGAGGATGAAAAACTGAAGAAG
CTGGTGGAACAGAATGGAACAGATGACTGGAAAGTTATTGCCAATTATCTCCCGAATCGAACAGATGTGCAGT
GCCAGCACCGATGGCAGAAAGTACTAAACCCTGAGCTCATCAAGGGTCCTTGGACCAAAGAAGAAGATCAGAG
AGTGATAGAGCTTGTACAGAAATACGGTCCGAAACGTTGGTCTGTTATTGCCAAGCACTTAAAGGGGAGAATT
GGAAAACAATGTAGGGAGAGGTGGCATAACCACTTGAATCCAGAAGTTAAGAAAACCTCCTGGACAGAAGAGG
AAGACAGAATTATTTACCAGGCACACAAGAGACTGGGGAACAGATGGGCAGAAATCGCAAAGCTACTGCCTGG
ACGAACTGATAATGCTATCAAGAACCACTGGAATTCTACAATGCGTCGGAAGGTCGAACAGGAAGGTTATCTG
CAGGAGTCTTCAAAAGCCAGCCAGCCAGCAGTGGCCACAAGCTTCCAGAAGAACAGTCATTTGATGGGTTTTG
CTCAGGCTCCGCCTACAGCTCAACTCCCTGCCACTGGCCAGCCCACTGTTAACAACGACTATTCCTATTACCA
CATTTCTGAAGCACAAAATGTCTCCAGTCATGTTCCATACCCTGTAGCGTTACATGTAAATATAGTCAATGTC
CCTCAGCCAGCTGCCGCAGCCATTCAGAGACACTATAATGATGAAGACCCTGAGAAGGAAAAGCGAATAAAGG
AATTAGAATTGCTCCTAATGTCAACCGAGAATGAGCTAAAAGGACAGCAGACACAGAACCACACATGCAGCTA
CCCCGGGTGGCACAGCACCACCATTGCCGACCACACCAGACCTCATGGAGACAGTGCACCTGTTTCCTGTTTG
GGAGAACACCACTCCACTCCATCTCTGCCAGCGGATCCTGGCTCCCTACCTGAAGAAAGCGCCTCGCCAGCAA
GGTGCATGATCGTCCACCAGGGCACCATTCTGGATAATGTTAAGAACCTCTTAGAATTTGCAGAAACACTCCA
ATTTATAGATTCTTTCTTAAACACTTCCAGTAACCATGAAAACTCAGACTTGGAAATGCCTTCTTTAACTTCC
ACCCCCCTCATTGGTCACAAATTGACTGTTACAACACCATTTCATAGAGACCAGACTGTGAAAACTCAAAAGG
AAAATACTGTTTTTAGAACCCCAGCTATCAAAAGGTCAATCTTAGAAAGCTCTCCAAGAACTCCTACACCATT
CAAACATGCACTTGCAGCTCAAGAAATTAAATACGGTCCCCTGAAGATGCTACCTCAGACACCCTCTCATCTA
GTAGAAGATCTGCAGGATGTGATCAAACAGGAATCTGATGAATCTGGAATTGTTGCTGAGTTTCAAGAAAATG
GACCACCCTTACTGAAGAAAATCAAACAAGAGGTGGAATCTCCAACTGATAAATCAGGAAACTTCTTCTGCTC
ACACCACTGGGAAGGGGACAGTCTGAATACCCAACTGTTCACGCAGACCTCGCCTGTGGCAGATGCACCGAAT
ATTCTTACAAGCTCCGTTTTAATGGCACCAGCATCAGAAGATGAAGACAATGTTCTCAAAGCATTTACAGTAC
CTAAAAACAGGTCCCTGGCGAGCCCCTTGCAGCCTTGTAGCAGTACCTGGGAACCTGCATCCTGTGGAAAGAT
GGAGGAGCAGATGACATCTTCCAGTCAAGCTCGTAAATACGTGAATGCATTCTCAGCCCGGACGCTGGTCATG
TGAGACATTTCCAGAAAAGCATTATGGTTTTCAGAACACTTCAAGTTGACTTGGGATATATCATTCCTCAACA
TGAAACTTTTCATGAATGGGAGAAGAACCTATTTTTGTTGTGGTACAACAGTTGAGAGCAGCACCAAGTGCAT
TTAGTTGAATGAAGTCTTCTTGGATTTCACCCAACTAAAAGGATTTTTAAAAATAAATAACAGTCTTACCTAA
ATTATTAGGTAATGAATTGTAGCCAGTTGTTAATATCTTAATGCAGATTTTTTTAAAAAAAACATAAAATGAT
TTATCTGTATTTTAAAGGATCCAACAGATCAGTATTTTTTCCTGTGATGGGTTTTTTGAAATTTGACACATTA
AAAGGTACTCCAGTATTTCACTTTTCTCGATCACTAAACATATGCATATATTTTTAAAAATCAGTAAAAGCAT
TACTCTAAGTGTAGACTTAATACCATGTGACATTTAATCCAGATTGTAAATGCTCATTTATGGTTAATGACAT
TGAAGGTACATTTATTGTACCAAACCATTTTATGAGTTTTCTGTTAGCTTGCTTTAAAAATTATTACTGTAAG
AAATAGTTTTATAAAAAATTATATTTTTATTCAGTAATTTAATTTTGTAAATGCCAAATGAAAAACGTTTTTT
GCTGCTATGGTCTTAGCCTGTAGACATGCTGCTAGTATCAGAGGGGCAGTAGAGCTTGGACAGAAAGAAAAGA
AACTTGGTGTTAGGTAATTGACTATGCACTAGTATTTCAGACTTTTTAATTTTATATATATATACATTTTTTT
TCCTTCTGCAATACATTTGAAAACTTGTTTGGGAGACTCTGCATTTTTTATTGTGGTTTTTTTGTTATTGTTG
GTTTATACAAGCATGCGTTGCACTTCTTTTTTGGGAGATGTGTGTTGTTGATGTTCTATGTTTTGTTTTGAGT
GTAGCCTGACTGTTTTATAATTTGGGAGTTCTGCATTTGATCCGCATCCCCTGTGGTTTCTAAGTGTATGGTC
TCAGAACTGTTGCATGGATCCTGTGTTTGCAACTGGGGAGACAGAAACTGTGGTTGATAGCCAGTCACTGCCT
TAAGAACATTTGATGCAAGATGGCCAGCACTGAACTTTTGAGATATGACGGTGTACTTACTGCCTTGTAGCAA
AATAAAGATGTGCCCTTATTTTACCTA
  (SEQ ID NO: 171)
MARRPRHSIYSSDEDDEDFEMCDHDYDGLLPKSGKRHLGKTRWTREEDEKLKKLVEQNGTDDWKVIANYLPNR
TDVQCQHRWQKVLNPELIKGPWTKEEDQRVIELVQKYGPKRWSVIAKHLKGRIGKQCRERWHNHLNPEVKKTS
WTEEEDRIIYQAHKRLGNRWAEIAKLLPGRTDNAIKNHWNSTMRRKVEQEGYLQESSKASQPAVATSFQKNSH
LMGFAQAPPTAQLPATGQPTVNNDYSYYHISEAQNVSSHVPYPVALHVNIVNVPQPAAAAIQRHYNDEDPEKE
KRIKELELLLMSTENELKGQQTQNHTCSYPGWHSTTIADHTRPHGDSAPVSCLGEHHSTPSLPADPGSLPEES
ASPARCMIVHQGTILDNVKNLLEFAETLQFIDSFLNTSSNHENSDLEMPSLTSTPLIGHKLTVTTPFHRDQTV
KTQKENTVERTPAIKRSILESSPRTPTPFKHALAAQEIKYGPLKMLPQTPSHLVEDLQDVIKQESDESGIVAE
FQENGPPLLKKIKQEVESPTDKSGNFFCSHHWEGDSLNTQLFTQTSPVADAPNILTSSVLMAPASEDEDNVLK
AFTVPKNRSLASPLQPCSSTWEPASCGKMEEQMTSSSQARKYVNAFSARTLVM*
```

An amino acid sequence for the protein encoded by the YO5A gene that is a regulator of T cell exhaustion/dysfunction resistance is available from the UniPROT database at accession no. Q9Y4I1, cDNA NM_001142495, with gene location at Chr 15: 52307281-52529132.

(SEQ ID NO: 172)
GGCGGCGGCGGTGGCGGCGGCGGCAGCGGAGGCAGCATCGCGGGCCGCCGAAGTGGCTGCGGCGGCGGCGGCG
ACCCCTAGGACCCCGGGCAGGTGGAGCGCTCGCGGCTGCTCGCGGCGAGCCCGGGAGTGATGGCGAGGCCCCT
GCGGGCGGCCAGCGCCTGAGGCGCCCCGCCCCGCCCCGCCCCGCGCGGCCCTCCCCGCCCGGCCCTGCCCTGC
CCTGCTCCCTGCCGGCGGCTGCGGGCGCTTCCTAGTCCGCTCGGGCGGCCGCCCAGGCGAGGTGCGGCCTCCG
CACAGGCGGGGGGCGTAGGCGCGCGGGGCCCGCCATGGCTGCGTCGGAGCTCTACACAAAGTTTGCCAGGGTT
TGGATACCTGATCCAGAGGAAGTCTGGAAGTCAGCAGAGCTGCTCAAAGATTATAAGCCAGGAGATAAAGTCC
TCCTGCTTCACCTCGAGGAAGGAAAGGATTTGGAATACCATCTAGATCCAAAGACCAAGGAGCTGCCTCACTT
ACGAAATCCTGACATACTTGTTGGTGAAAATGACCTCACAGCCCTCAGCTATCTTCATGAGCCTGCTGTGCTC
CATAATCTCAGAGTCCGCTTTATTGATTCCAAACTTATTTATACGTATTGTGGTATAGTCCTAGTAGCTATAA
ATCCCTATGAACAGCTGCCTATTTATGGAGAAGATATTATTAATGCATACAGTGGTCAGAACATGGGTGATAT
GGATCCACATATCTTTGCAGTAGCTGAAGAAGCTTACAAGCAAATGGCCAGAGATGAACGAAATCAGTCCATC
ATCGTAAGTGGAGAGTCTGGGGCAGGAAAAACAGTCTCAGCTAAGTATGCCATGCGATACTTTGCAACTGTGA
GTGGTTCTGCCAGTGAGGCCAATGTGGAGGAAAAGGTCTTGGCCTCCAACCCCATCATGGAGTCCATTGGAAA
TGCTAAAACAACCAGGAATGATAATAGCAGCCGTTTTGGGAAGTATATTGAGATTGGTTTTGATAAGAGATAT
CGAATCATTGGTGCCAATATGAGAACTTATCTTTTAGAGAAATCCAGAGTGGTATTCCAGGCAGAAGAGGAGA
GAAACTATCATATCTTCTATCAGCTTTGTGCCTCAGCAAAGTTACCTGAATTTAAAATGCTACGATTAGGAAA
TGCAGATAACTTTAATTACACAAAACAAGGAGGCAGTCCTGTGATTGAAGGAGTGGATGATGCAAAGGAGATG
GCACATACTAGGCAGGCCTGCACTTTGCTAGGAATTAGTGAATCTCATCAAATGGGAATTTTCCGAATACTTG
CTGGCATCCTTCACTTAGGCAATGTTGGATTTACATCCCGAGATGCAGACAGCTGCACAATACCTCCCAAGCA
TGAACCTCTCTGCATCTTCTGTGAACTCATGGGTGTGGACTATGAGGAGATGTGTCACTGGCTCTGCCATCGG
AAACTGGCTACTGCCACAGAGACATACATCAAGCCCATCTCCAAGCTGCAGGCCACGAATGCCCGCGATGCTT
TGGCCAAGCACATCTATGCCAAGCTCTTTAACTGGATTGTAGATAATGTCAATCAGGCTCTCCATTCTGCTGT
CAAACAGCACTCTTTTATTGGTGTGCTAGACATTTACGGATTTGAAACATTTGAGATAAATAGTTTTGAACAG
TTTTGCATAAATTATGCAAATGAAAAACTACAGCAACAATTCAATATGCATGTCTTCAAATTGGAGCAAGAAG
AATATATGAAGGAACAAATTCCATGGACACTCATAGATTTTTATGATAATCAGCCTTGTATTAATCTTATAGA
ATCAAAACTAGGCATTCTAGATTTACTGGATGAGGAATGCAAGATGCCTAAAGGCACAGATGACACCTGGGCC
CAAAAATTGTACAACACACATTTGAACAAATGTGCACTCTTTGAAAAGCCTCGTCTATCAAACAAAGCTTTCA
TCATCCAACATTTTGCTGACAAAGTGGAATACCAGTGTGAAGGATTTCTCGAAAAGAATAAAGACACCGTTTT
TGAAGAACAAATTAAAGTTCTTAAATCAAGCAAGTTTAAGATGCTACCAGAACTATTTCAAGATGATGAGAAG
GCCATCAGTCCAACTTCAGCCACCTCCTCAGGGCGCACACCCCTCACACGAACTCCTGCAAAGCCCACCAAAG
GCAGACCAGGCCAAATGGCCAAAGAGCACAAGAAAACAGTGGGGCATCAGTTCAGAAACTCCCTGCACCTGCT
TATGGAGACACTCAATGCCACTACCCCTCACTATGTGCGCTGTATCAAGCCTAATGACTTCAAGTTCCCATTC
ACGTTTGATGAGAAGAGGGCAGTGCAGCAGCTGAGAGCATGTGGTGTCCTGGAAACCATCCGAATCAGTGCGG
CCGGTTTCCCCTCACGGTGGACTTACCAAGAATTTTTCAGCCGCTACCGTGTCCTAATGAAGCAGAAAGATGT
GCTGAGTGACAGAAAGCAAACATGCAAGAATGTGTTAGAGAAACTGATACTGGACAAGGACAAATACCAGTTT
GGTAAGACAAAGATCTTTTTCCGTGCCGGTCAAGTGGCCTATCTAGAAAAATTGAGAGCTGACAAACTGAGAG
CTGCCTGCATCCGGATCCAGAAGACCATCCGAGGGTGGCTGCTGAGAAAGAAGTACCTACGCATGCGGAAGGC
AGCCATCACCATGCAGAGATACGTGCGGGGCTACCAGGCCCGATGCTATGCTAAGTTTCTGCGCAGAACCAAG
GCAGCAACCATCATTCAAAAGTACTGGCGCATGTATGTGGTCCGCAGGAGGTACAAGATTAGACGAGCTGCCA
CTATCGTTCTTCAGTCTTACTTGCGAGGCTTCTTGGCCAGAAATAGGTATCGCAAGATACTCCGTGAGCACAA
AGCAGTCATCATTCAGAAGCGAGTCCGGGGCTGGCTGGCCCGCACACACTACAAGAGGAGCATGCATGCCATC
ATCTACCTTCAGTGCTGCTTCAGGCGGATGATGGCCAAGCGTGAGCTAAAGAAGCTCAAAATCGAGGCTCGCT
CAGTGGAGCGCTATAAGAAGCTGCACATCGGCATGGAGAACAAGATCATGCAGCTGCAGCGCAAAGTTGATGA
GCAGAACAAAGACTACAAATGCCTTGTGGAGAAACTAACCAATCTGGAAGGAATATACAACTCTGAGACTGAG
AAACTACGAAGTGACTTAGAACGTCTTCAACTAAGTGAAGAGGAAGCGAAAGTTGCCACTGGGCGGGTCCTTA
GTCTGCAGGAAGAAATTGCCAAGCTCCGGAAAGACCTGGAGCAAACTCGTTCAGAGAAAAAATGCATTGAGGA
ACATGCAGATCGATACAAACAAGAAACAGAGCAGCTGGTATCAAATCTGAAGGAAGAAAATACTTTGCTGAAG
CAAGAAAAAGAAGCCCTCAATCACCGCATCGTGCAGCAGGCTAAGGAGATGACAGAAACTATGGAGAAGAAGT
TAGTAGAAGAAACGAAACAACTGGAACTCGACCTTAATGATGAAAGGCTGAGATATCAGAACCTTCTGAATGA
GTTCAGTCGCCTGGAAGAAAGATATGATGACCTCAAGGAAGAGATGACCCTTATGGTGCATGTGCCTAAGCCT
GGACACAAGAGAACAGACTCCACCCACAGCAGCAACGAGTCTGAATATATCTTTAGCTCTGAAATTGCAGAAA
TGGAAGACATTCCATCAAGGACAGAGGAACCAAGTGAGAAGAAGGTACCTCTGGACATGTCATTGTTCCTTAA
GCTCCAGAAGCGGGTCACAGAGCTGGAGCAGGAGAAGCAGGTGATGCAGGATGAGCTGGACCGCAAGGAGGAG
CAGGTGCTCCGCAGCAAGGCCAAGGAAGAAGAAAGACCACAAATTAGAGGTGCAGAACTGGAATATGAGTCAC
TCAAGCGTCAAGAACTAGAATCAGAAAACAAAAAACTGAAGAATGAGCTAAATGAGTTGCGCAAGGCCCTCAG
TGAGAAAAGTGCCCCAGAGGTGACCGCCCCAGGTGCACCTGCCTACCGTGTCCTCATGGAGCAGCTGACCTCT
GTGAGCGAGGAGCTTGATGTCCGCAAGGAGGAAGTCCTCATCTTAAGGTCTCAACTGGTGAGCCAGAAAGAGG
CCATCCAACCCAAGGATGACAAGAATACAATGACAGATTCCACAATACTTTTGGAAGATGTACAAAAAATGAA
AGATAAAGGTGAAATAGCACAAGCATACATTGGTTTGAAAGAAACAAATAGGCTCCTGGAATCCCAGCTGCAG
TCACAGAAGAGGAGCCATGAGAATGAGGCCGAGGCCCTCCGTGGGGAGATCCAGAGCCTGAAGGAGGAGAACA
ACCGACAGCAGCAGCTGCTGGCCCAGAACCTGCAGCTGCCCCCAGAGGCCCGCATTGAGGCCAGCCTGCAGCA
CGAGATCACCCGGCTGACCAACGAAAACTTGGATTTGATGGAACAACTTGAAAAACAGGATAAGACGGTCCGT
AAACTGAAAAAACAACTGAAAGTATTTGCCAAAAAAATTGGCGAACTAGAAGTGGGCCAGATGGAGAACATAT
CCCCAGGACAGATCATTGATGAACCCATCCGACCAGTCAACATTCCCAGGAAAGAAAAGGATTTCCAAGGGAT
GCTGGAATACAAGAAGGAGGATGAGCAAAAACTTGTTAAGAACCTGATTCTGGAACTGAAGCCACGTGGTGTA
GCAGTCAATTTGATTCCAGGATTACCGGCATATATCCTGTTCATGTGTGTTCGACATGCTGACTACCTGAATG
ATGATCAGAAAGTAAGGTCGTTGCTAACATCAACAATTAACAGCATCAAAAAAGTATTGAAGAAAAGAGGTGA
TGATTTTGAAACCGTCTCCTTCTGGCTCTCTAACACATGCCGATTTTTGCACTGCTTGAAACAGTACAGTGGA
GAAGAGGGCTTTATGAAGCACAACACATCTCGCCAGAATGAACACTGCCTCACCAATTTTGACCTGGCTGAGT
ATCGGCAGGTGCTGAGTGACTTGGCCATTCAGATCTACCAGCAGCTCGTGCGGGTGTTAGAGAACATCCTTCA
GCCAATGATTGTCTCAGGCATGCTGGAACATGAAACGATTCAGGGCGTGTCTGGGGTGAAGCCCACAGGGTTG
AGAAAGCGAACCTCCAGTATCGCCGATGAGGGCACCTACACACTGGACTCCATCCTCCGGCAGCTCAACTCCT
TCCACTCGGTCATGTGTCAGCATGGCATGGACCCTGAACTGATCAAGCAGGTGGTCAAGCAGATGTTCTACAT
CATAGGGGCCATCACCCTGAACAACCTTCTCCTGCGGAAGGACATGTGCTCCTGGAGTAAAGGCATGCAGATC
AGGTACAATGTCAGTCAACTGGAAGAATGGCTGCGTGACAAGAATCTGATGAATAGTGGGGCTAAAGAAACCC
TGGAACCTCTCATTCAGGCTGCTCAACTTTTGCAAGTGAAAAAGAAAACAGATGATGATGCAGAAGCCATTTG
TTCTATGTGCAATGCTTTAACTACTGCCCAGATTGTGAAAGTGTTGAATTTGTATACTCCAGTTAATGAGTTT
GAAGAAAGAGTCTCTGTGTCGTTCATTCGTACTATACAGATGCGTTTACGAGACAGGAAAGACTCTCCCCAGC
TGCTCATGGATGCTAAACACATCTTTCCTGTCACCTTTCCTTTCAACCCATCTTCCCTCGCACTAGAAACCAT
CCAGATTCCAGCCAGCCTCGGCCTGGGCTTCATTTCACGGGTCTGAAAGTGATGTCCAGGCAAAAATTGACAA
TACATTTCTTGCCCGAAATAAGAACCCATTATTTCCAGTGAGTTACTGAAAATACATTTTTAAAGAGAAAGTA
CTGATTATCTCCCAAATGAGAAGTCATTAACTGGAAATCTCCCTAGAATACTTTCATCACTTTGGAAACAAAG
ATAGGCTCTTTCGTGCTGTGTTATCTTTATAGCAACACTCATCCTTAACCAACTAGGTACCGTGAGTTTACAT
ACAGGAGAATGATGGAAGGAAGGGAGGAAGGAAAGGAGGAGAAAAATGTGTCTTCAGCTGGCAGCATTTATTT
TAAATCCTTAGCACTGAGTTTGAATGGTATAAAAAGTATAACTTCCATAGATGAGCTGTTGTTAGGAAGGCAC
CAAAGAACCTCCTCTGCACTAAACAGGAGAATGGAAAGAAAAGTCTCCATTGAGTACATATCATGTCAGTTTA
GTAATCAATTATGTTGATATTGTTAAACTGGTTCAAAGAAATAAACTGGCAATATGTAAAGTAATTCCTCATT
TGTGTCACTATGATATAGAGATATTAAAGGAATGTTGGTTTGCTAAATAGTATAGATGTCCATTTGTACTATA
GTTTACTGAGCATTTTAAATTGCTGCTACATACTGTCTTCTTAAAATGTAAGTGATATTAGGCACTACAATAA
GTTTCTCTTGTCAATTCTGTTTACAATTCAATCAGATCACAGTTTTAACTGGATTATATGCAAATACCTACAG
ATTCACCTGCACAAGTAGCAGACACTGGAAAGTCATGTAGTAATATGACAAAATGCTTGACATTTAGGGGTAG
GATTAGACAAAGTGGCTATTGTTGATGTCATTATTTATTCAGGATGTATTACATTGATGTGCTCATTAATTTT
CCCTGGGTGGATATTGCGTCAGGGTACAGTGTTCTGTGAAGTGACTTATTTTTAACTACCAGATCTGATTCCT
TCAGTGCATATTTTCAACCTTGACAGGTTTTCTCTCTTCTTAATTTATTAAGAATTAATCTCGGCTGGGCGCG
GTGGCTCACGCCTGTAATCCCAGCACTTTGGGAAGCCAAGGTGGGCGGATCACTTCAGGTTAGGAGTTGGAGA
CCAGCCTGGCCAACATGGCGAAACCCTGTCTCTACTAAAAATACAAAAATTAGCCGGGCGTGGTGGCACTTGC
CTGTAATCCCAGCTACTCGGGAGGCTGAGGCACGAGAATCGATTAAACCTGGGAGGCGGAGATTGCAATGAGA
TCGAACCACTGCACTCCAGCCTGGGTGACAGAGAGAGACACTGCCTTGGAAAAAAAAAGAATCTCACTCACTA
TCTAGAGAGGATTGTCAGAATATTCACGATTCAGGTCTTGAAACTTTGATTATGCAAAAGAAGGTATATAATA
AATATTTCATTATGATTCAGTTTTTAAGGCTTTGCAGCTTCTATAAGTGTTCTCAGATGCCACTAGATAATTT
TAAAAGCATCATATTAGAAATACTTTAAGAAGACTTATATAAGAAATAGAAGATTGTTGAATTTTACAGAGGA
TTTGGTTCATTAAGACCCAGATTCTGTAAGTTTTCATTCTGAAATTCTAGTTAAACATATTCACCATTTTTCT
TAGGAATCTTATACAATAAATCCTTCAGGTTGCACAAAAGCAAATTATTAGTTTTCATTAGAAACTCTGGTTC
TGAATTACAATCATAGGTTATATAAATTTAACTGTTAGATGGTCTATAAATCTTATTAAAATATGTGCAATAT
TTATGGAAGTCAAACAGCTTCATATCAGTGATAAAGATTGTTATTAAAAGATAAATACTGTCTGTTAATTTAC
ATGGGCCTCAAGTTCCTCGTTTATAAAATAAGAGAGTTGGACACTGATTCTTAACATCTCCTCCACATTTAAA
ATTCTCTCTTCTCAGCCCTTAGATTCTAGAGAGAAAAAGCTGCAGTTACTCAGTAAGTCCATTCTCTGATGGA
AAGACCAGTGTGTAGTGCCTGTCAATTCCTTAGGATTAATCAAATGTAAAATCACAAGTTTGTGTAGCTGTAA
CCTTTCTTAAATGTACATGATTTATGTACATGCTTTTAGAAGGTCCTACTATATTTGTATTATAATTAGTTTA
AGTAATTTTTATTACATCATGTATTGCTTTATTCAGTTTGAATACATTTATTTATTTATTTGCAGTATCAACC
AGAAACACTACCAATTGCATCAAATTCTCCCAGTTTTTCCTGGTTGTCAATGCGGTTTTCAATGCACAATTAA
GTCATAGCCATTTGGTTCGTACCAAATGTGTCAGAATCTAACAGCATCCGATAGGCTGTAAGTTGGGGAGTTG
CTAAGAAAATGCAACGTGGTACAGGCTGTCCGCCTCAGCCCTGGAAATCTCCCAGACCTCCCCCAGCTTCATC
CTGTGTAGCACGACTCAACGTGCACCCTGAATCTTCTCAGGTCTTCCAGGTCATGCTGTAGCTGTCACTGCCA
TGCAGCCCTTTTTTTTACTCCGGACAGCTCATGTACTGAAGCGTCATGAAAGAAAGGCTGTGGTCTGAGCCCT
TCTCTCCCATCTCCTGTCTTTGTCCTGTCAAGTGCTGGAGCCAGAGCTCCTACAGCTGCCCTTGGTGGTTTCT
CCTGTTCAGCGATGGTGGCACAAAGGTTCTGCTATTCCAGGGCTCCAGCTTCCTCCCAGGTCTACCCAGAGCT
CCAGATGGGGGTCTGAATTAACCTCTCTTGGTGGCCTGGAGATTTTTAGTCATTGACAAGAATACCTTGTAAC
CAGGGAACCCCAAGGCCCAGTAAATGATTCTGTATACCATTTTCTTGAAGGTACAAGAAGATTCTGCCGACTA
TGGGGATCTTTGGGCCAGTTTGAGGATTGCTTTCCCTCTGAGGTTCTTTCTCTCTGTCAGCCACACTTTCTCA
CCCAACTTCAGACACACCCTGCCAGCCTTTCCCCTACTCATTCACTCTTCCCCTTCCCTCAACTTAATCGTCT
ATCCCGTTGCCTGCTGTTTGACTGTGCACTGAAGGCAGGTGGATGGAGTCAGTCCTCAGTTGCCCCTGCTGGC
CTTCCTGGTGCTTACCATCAGCCCAATCTTTGCACAGTCCTTGTTGTTCTTACTTCTCTGCATGCATTCCTTC
AGAAGATCAGTCATCAACTTTTTCTTAATTCCTCTGTGACACACAATGGGAATTCAAAGGAAGAGATCTTAAA
AGTCACAACAGTTCTTTATCTTAATAATCCCCTCCCCATTCACCTTACTACATGCAGACTCACCTCACACCCT
TACAACTTGAAGCTGAAAATTTAAAAGTAATTTCCCTTTTTGCAGCTTTTCCTCAGGTTAAGGCTTTGATCTG
CCTGAGAGTAACTCTAAAAGGAGGGAAGATAAATATGGGATAAAATCCACAAAGTGTAGCTTCTAATTCCTTT
GGAAGTTTAAAAAATTTCCACATATCTGATGCTTCTTTTGTCAGGTGCAGAAGCACAAAAACATATTCCGAAG
CCAACTGATAGGGAATTTGGGGATTATTGTCAGTTTGGAGAATTTGCTGTGTTATTTCTTCATTTCCATGGAT
AGCTCATAGTTGGCTCTTTCTGGGTGAGTAATTATGTGTAATATAGATCAAATCTTTTACTAAGGTTACAGCT
ACATGTTAGGGGAGGCTATGAAAATACTATATTATTATAATTTCAGTGCAGTGATTGTTGTGAGAAATAACTT
TCATGGTAACCCTAGGAAAATGGGCACCTGCCACCATCCTGAGAAGTCCTCACACAATGCCCTTTCTCTCTTA
CACACACACACACACACATACACACACACACACCCCCGTCACTAATTCATAGAGTTCCTTAGCAGGCATAGTC
AAGGATCCTCTGGGTAATGTCAGCTGCTTAGTGATAAAACAGAGCCAAAACTAGTGCATCCTGTTGAAAGTAA
TGCAGAAACAGTACCTGGGTCCAGATATGCTTTCCTGCGGCGCTTTCCTCTGTTACCTCGTTTCATCCTCACA
GCAGCATGGACGGTAGGTGGGGTCGCTTCTACAATCATTTCTGATGATAGCTTGGGAATAGAGATAGGGGCAG
TGACTTGCCTGATGTCGCACAGCCCTCTGGCTGTCCTGCTTTCCCATATGGAGCAGTGGTGGTGTGGGCACCT
GTGATGCAGGAGACTTTAAAAATGTCGTGAGGTCACGTGCTGCCCCTCCTGGTACGTGTGGAATGCCCCTGGC
CAGCAAGGGGTGCTTTTTTATCAGAGTTGGCAGCTGGCATGTGGGAACCGAGCAAGTGCTGCGTACCAAGTTA
CTTGTTTTAAGGAGACCAAGTGCTCAGCGCCAGGTGGTTTTCTTTTTTGTCATAGTTACTTGCTATAACTCAG
CTTGACTTCTGTCATGAATCAGTGCTCTCTGGGAGGATGCAATACTCTGTTTGGGCATTAATTGGTAGCAGGT
TGTCTCAACCAAAAAGACAGGAAACAGCAAAAGCCTCTCTGAAATTAAGAGGAAAGTTACTCTCCCCACACCC
ATCAGAGTCTTTATTGGAGCCACCAGGTGAGCTGTGCAGCCTGGACAGGCCTGCAGCTATAGGCCACCTTCCC
AGTTTAGGTCCTCAGCACAGGGGAGCCCAAGTCACTGGGTGCCTTCCGAGGGCTGTCACTGGGCAGGCCATAT
ACAAGTCAGTGTGTGCGTGGGCACTGCAGTGTGTGCATGCCGTAGGTGTTGATGGGTGCTAGGAGGGGTGTCG
TGTGCATGCGCGTTGAAGAGGATCTGTATTGCCGTGACCTCTGTTCATGGATGAGTGCATTGTAATTTGTTCT
CAGGCTGTGCTGTGAGGGCCGCCTTAACCCTTGCTCCCTTCCCTTCTAGAGCTGCCTTAAGTTCTCCAGAACT
TTTCTTCTGTAAAGGATATCTTGCCTGGAAGGGATATCTTGCCCTGTTTCTCAAGGTTTTGTGAGAGTTTTGA
CTGGATGTGGCCCTGCATGACCCTCCTTCTCCTGTACTTCCTCTTTCCTTTCCAAATGGGAATTAGAACTGTG
GGGCAGCAACAGTCTCAGAGCCAGTGAGAGGCCAGCTTAGAGAATGCTTCTGAGTTAGTGGGACTCTGTGTCA
CAAGTAAGCAAATGAATATATGAAAGAAATTATGGAGATAAGTTAGATTCTTGGTAATACTTAAATGTCTTGC
TTTCTACTAACCTTTTGTTACTAAAGGTAAAGGGTATAACTCAAACTTTTTGTGGACATTCTTTTCAAAATTT
TTTAAGAACCCTGTACTATAAAAGGTTGAGTAAAAACAGGAAAGCGTGCTATAAGTTCAAATCTGTTGTATTA
CCCTAAATTAGATAAACCAACCTGAATTATAGTAGATTTCTCAATAGATGAGGAACTGAAAAATACTATGTAA
AATATCTTCCAAAATGCTTTTTATACTTTTTTTATTTGTAATTTGGTCTATCTAAAATGTTCGTTAGCTTAAC
TTAATGGGCGTTATTGGATTCATATGACTAACGTTTCCTCAGTATTGTAATGCTTGAAATATTTGAAAGAAAA
AATGTTGTTTTTTAGTTGAAACTGGTATATATAATTCAGTGCTTGGCAGGTTAGTATATTTTTATGCATTTTT
CAGAGTCAGCAGTTTCAAATCTTATTGTTATCATGTTATAAAATTTTAGCCCACATTTCAGGCTCCGTAAATC
ATTTGAGCCATTATTTTTTCCCAACAAATGGTGAATTTTTTCTTTAAATGTGGATATATATGTTGTAATTTAT
GATTCCTGGTTATGTATTTTTGTGGGATCCTGCAGTAAAATTGACTTTTTTGTGTCTTTGGGAGATTTAAATT
GCGCTAACAGTGTTGCGCAAAAATGAGTTCATGCCATTTAACATATTGTATTTTAATTATTAACTGTATTAAT
TTACTATGAAATGGACATCCTTTTAACTAAAATGGAATTGAACATTGCAGTTTTCAAATATTTTTCCTTGTTG
GGTCTGGAAAAGGAATTCTACTTTGATCTGCATAGAAAATTTTGATACAATTTTTTGAAAGTTCTTAGGTGAA
ACATTTACCCATTAAAAAGGAAGCAGAAATACTGAGACATGAAAGGCATTATCAACTAACTCTAGACTCTAGA
ACCCATTCTAGCATATCTCACGTGCAATTTTTAAAAATAAGTTAATAATTCATCTCATATCAACAAAAGCCTT
TGAAACATGGGTTTTCACTAGATATCACCTAGTGCTAAGATAAAAACCAAAACAATATCAGAATTACATTTAT
GCTCTAAATTTGTAGTTGTCCATTGTTGTGCTTAGTAAATGTGTGTCATTAATGCTGTATTCTCCTAGCTATT
ATGGAAACTTGTTTAAATAAAGATATGGATAT
(SEQ ID NO: 173)
MAASELYTKFARVWIPDPEEVWKSAELLKDYKPGDKVLLLHLEEGKDLEYHLDPKTKELPHLRNPDILVGEND
LTALSYLHEPAVLHNLRVRFIDSKLIYTYCGIVLVAINPYEQLPIYGEDIINAYSGQNMGDMDPHIFAVAEEA
YKQMARDERNQSIIVSGESGAGKTVSAKYAMRYFATVSGSASEANVEEKVLASNPIMESIGNAKTTRNDNSSR
FGKYIEIGFDKRYRIIGANMRTYLLEKSRVVFQAEEERNYHIFYQLCASAKLPEFKMLRLGNADNFNYTKQGG
SPVIEGVDDAKEMAHTRQACTLLGISESHQMGIFRILAGILHLGNVGFTSRDADSCTIPPKHEPLCIFCELMG
VDYEEMCHWLCHRKLATATETYIKPISKLQATNARDALAKHIYAKLFNWIVDNVNQALHSAVKQHSFIGVLDI
YGFETFEINSFEQFCINYANEKLQQQFNMHVFKLEQEEYMKEQIPWTLIDFYDNQPCINLIESKLGILDLLDE
ECKMPKGTDDTWAQKLYNTHLNKCALFEKPRLSNKAFIIQHFADKVEYQCEGFLEKNKDTVFEEQIKVLKSSK
FKMLPELFQDDEKAISPTSATSSGRTPLTRTPAKPTKGRPGQMAKEHKKTVGHQFRNSLHLLMETLNATTPHY
VRCIKPNDFKFPFTFDEKRAVQQLRACGVLETIRISAAGFPSRWTYQEFFSRYRVLMKQKDVLSDRKQTCKNV
LEKLILDKDKYQFGKTKIFFRAGQVAYLEKLRADKLRAACIRIQKTIRGWLLRKKYLRMRKAAITMQRYVRGY
QARCYAKFLRRTKAATIIQKYWRMYVVRRRYKIRRAATIVLQSYLRGFLARNRYRKILREHKAVIIQKRVRGW
LARTHYKRSMHAIIYLQCCFRRMMAKRELKKLKIEARSVERYKKLHIGMENKIMQLQRKVDEQNKDYKCLVEK
LTNLEGIYNSETEKLRSDLERLQLSEEEAKVATGRVLSLQEEIAKLRKDLEQTRSEKKCIEEHADRYKQETEQ
LVSNLKEENTLLKQEKEALNHRIVQQAKEMTETMEKKLVEETKQLELDLNDERLRYQNLINEFSRLEERYDDL
KEEMTLMVHVPKPGHKRTDSTHSSNESEYIFSSEIAEMEDIPSRTEEPSEKKVPLDMSLFLKLQKRVTELEQE
KQVMQDELDRKEEQVLRSKAKEEERPQIRGAELEYESLKRQELESENKKLKNELNELRKALSEKSAPEVTAPG
APAYRVLMEQLTSVSEELDVRKEEVLILRSQLVSQKEAIQPKDDKNTMTDSTILLEDVQKMKDKGEIAQAYIG
LKETNRLLESQLQSQKRSHENEAEALRGEIQSLKEENNRQQQLLAQNLQLPPEARIEASLQHEITRLTNENLD
LMEQLEKQDKTVRKLKKQLKVFAKKIGELEVGQMENISPGQIIDEPIRPVNIPRKEKDFQGMLEYKKEDEQKL
VKNLILELKPRGVAVNLIPGLPAYILFMCVRHADYLNDDQKVRSLLTSTINSIKKVLKKRGDDFETVSFWLSN
TCRFLHCLKQYSGEEGFMKHNTSRQNEHCLTNEDLAEYRQVLSDLAIQIYQQLVRVLENILQPMIVSGMLEHE
TIQGVSGVKPTGLRKRTSSIADEGTYTLDSILRQLNSFHSVMCQHGMDPELIKQVVKQMFYIIGAITLNNLLL
RKDMCSWSKGMQIRYNVSQLEEWLRDKNLMNSGAKETLEPLIQAAQLLQVKKKTDDDAEAICSMCNALTTAQI
VKVLNLYTPVNEFEERVSVSFIRTIQMRLRDRKDSPQLLMDAKHIFPVTFPFNPSSLALETIQIPASLGLGFI
SRV*

An amino acid sequence for the protein encoded by the NFKBIA gene that is a regulator 60 of T cell exhaustion/dysfunction resistance is available from the UniPROT database at accession no. P25963, cDNA NM_020529, with gene location at Chr 14: 35401079-35404749.

(SEQ ID NO: 174)
ACAGCAGTCCGTGCCGCCGTCCCGCCCGCCAGCGCCCCAGCGAGGAAGCAGCGCGCAGCCCGCGGCCCAGCGC
ACCCGCAGCAGCGCCCGCAGCTCGTCCGCGCCATGTTCCAGGCGGCCGAGCGCCCCCAGGAGTGGGCCATGGA
GGGCCCCCGCGACGGGCTGAAGAAGGAGCGGCTACTGGACGACCGCCACGACAGCGGCCTGGACTCCATGAAA
GACGAGGAGTACGAGCAGATGGTCAAGGAGCTGCAGGAGATCCGCCTCGAGCCGCAGGAGGTGCCGCGCGGCT
CGGAGCCCTGGAAGCAGCAGCTCACCGAGGACGGGGACTCGTTCCTGCACTTGGCCATCATCCATGAAGAAAA
GGCACTGACCATGGAAGTGATCCGCCAGGTGAAGGGAGACCTGGCCTTCCTCAACTTCCAGAACAACCTGCAG
CAGACTCCACTCCACTTGGCTGTGATCACCAACCAGCCAGAAATTGCTGAGGCACTTCTGGGAGCTGGCTGTG
ATCCTGAGCTCCGAGACTTTCGAGGAAATACCCCCCTACACCTTGCCTGTGAGCAGGGCTGCCTGGCCAGCGT
GGGAGTCCTGACTCAGTCCTGCACCACCCCGCACCTCCACTCCATCCTGAAGGCTACCAACTACAATGGCCAC
ACGTGTCTACACTTAGCCTCTATCCATGGCTACCTGGGCATCGTGGAGCTTTTGGTGTCCTTGGGTGCTGATG
TCAATGCTCAGGAGCCCTGTAATGGCCGGACTGCCCTTCACCTCGCAGTGGACCTGCAAAATCCTGACCTGGT
GTCACTCCTGTTGAAGTGTGGGGCTGATGTCAACAGAGTTACCTACCAGGGCTATTCTCCCTACCAGCTCACC
TGGGGCCGCCCAAGCACCCGGATACAGCAGCAGCTGGGCCAGCTGACACTAGAAAACCTTCAGATGCTGCCAG
AGAGTGAGGATGAGGAGAGCTATGACACAGAGTCAGAGTTCACGGAGTTCACAGAGGACGAGCTGCCCTATGA
TGACTGTGTGTTTGGAGGCCAGCGTCTGACGTTATGAGCGCAAAGGGGCTGAAAGAACATGGACTTGTATATT
TGTACAAAAAAAAAGTTTTATTTTTCTAAAAAAAGAAAAAAGAAGAAAAAATTTAAAGGGTGTACTTATATCC
ACACTGCACACTGCCTGGCCCAAAACGTCTTATTGTGGTAGGATCAGCCCTCATTTTGTTGCTTTTGTGAACT
TTTTGTAGGGGACGAGAAAGATCATTGAAATTCTGAGAAAACTTCTTTTAAACCTCACCTTTGTGGGGTTTTT
GGAGAAGGTTATCAAAAATTTCATGGAAGGACCACATTTTATATTTATTGTGCTTCGAGTGACTGACCCCAGT
GGTATCCTGTGACATGTAACAGCCAGGAGTGTTAAGCGTTCAGTGATGTGGGGTGAAAAGTTACTACCTGTCA
AGGTTTGTGTTACCCTCCTGTAAATGGTGTACATAATGTATTGTTGGTAATTATTTTGGTACTTTTATGATGT
ATATTTATTAAACAGATTTTTACAAA
(SEQ ID NO: 175)
MFQAAERPQEWAMEGPRDGLKKERLLDDRHDSGLDSMKDEEYEQMVKELQEIRLEPQEVPRGSEPWKQQLTED
GDSFLHLAIIHEEKALTMEVIRQVKGDLAFLNFQNNLQQTPLHLAVITNQPEIAEALLGAGCDPELRDERGNT
PLHLACEQGCLASVGVLTQSCTTPHLHSILKATNYNGHTCLHLASIHGYLGIVELLVSLGADVNAQEPCNGRT
ALHLAVDLQNPDLVSLLLKCGADVNRVTYQGYSPYQLTWGRPSTRIQQQLGQLTLENLQMLPESEDEESYDTE
SEFTEFTEDELPYDDCVFGGQRLTL*

An amino acid sequence for the protein encoded by the PAK2 gene that is a regulator of T cell exhaustion/dysfunction resistance is available from the UniPROT database at accession no. Q13177, cDNA NM_002577, with gene location at Chr 3: 196739857-196832647.

(SEQ ID NO: 176)
GTCTTCCTCCCCCAGGGTTGTGGCCACGCGCAGCGGCGGCGGTTGTTCCGCTTCCCCTCCGGCCCGGGCCGTC
GCCATTGCCGAAGGCTCCCTCCCCTCCCCTCCCTGGCGTGCGCAGGACTCCGCCGCCGCTGGGCCTAGCGGTA
GCAGCGGCTGCTCCAGCGCGGCGTCTCTTCCCGCCCCGCTTCCCCTTCCCTCCCCTCCCCTCCCCGCACCGCG
CGCTAGCCCGGGGCGGCTCCGCAGCCCGCCGGGAGCTCTGACCGAGGCGCCTCGCTGGGGCGGGGACCTTGCC
TTGCCCGGGGCCATTTCATAATTCTGAATCATGTCTGATAACGGAGAACTGGAAGATAAGCCTCCAGCACCTC
CTGTGCGAATGAGCAGCACCATCTTTAGCACTGGAGGCAAAGACCCTTTGTCAGCCAATCACAGTTTGAAACC
TTTGCCCTCTGTTCCAGAAGAGAAAAAGCCCAGGCATAAAATCATCTCCATATTCTCAGGCACAGAGAAAGGA
AGTAAAAAGAAAGAAAAGGAACGGCCAGAAATTTCTCCTCCATCTGATTTTGAGCACACCATCCATGTTGGCT
TTGATGCTGTTACTGGAGAATTCACTGGCATGCCAGAACAGTGGGCTCGATTACTACAGACCTCCAATATCAC
CAAACTAGAGCAAAAGAAGAATCCTCAGGCTGTGCTGGATGTCCTAAAGTTCTACGACTCCAACACAGTGAAG
CAGAAATATCTGAGCTTTACTCCTCCTGAGAAAGATGGCTTTCCTTCTGGAACACCAGCACTGAATGCCAAGG
GAACAGAAGCACCCGCAGTAGTGACAGAGGAGGAGGATGATGATGAAGAGACTGCTCCTCCCGTTATTGCCCC
GCGACCGGATCATACGAAATCAATTTACACACGGTCTGTAATTGACCCTGTTCCTGCACCAGTTGGTGATTCA
CATGTTGATGGTGCTGCCAAGTCTTTAGACAAACAGAAAAAGAAGACTAAGATGACAGATGAAGAGATTATGG
AGAAATTAAGAACTATCGTGAGCATAGGTGACCCTAAGAAAAAATATACAAGATATGAAAAAATTGGACAAGG
GGCTTCTGGTACAGTTTTCACTGCTACTGACGTTGCACTGGGACAGGAGGTTGCTATCAAACAAATTAATTTA
CAGAAACAGCCAAAGAAGGAACTGATCATTAACGAGATTCTGGTGATGAAAGAATTGAAAAATCCCAACATCG
TTAACTTTTTGGACAGTTACCTGGTAGGAGATGAATTGTTTGTGGTCATGGAATACCTTGCTGGGGGGTCACT
CACTGATGTGGTAACAGAAACGTGCATGGATGAAGCACAGATTGCTGCTGTATGCAGAGAGTGTTTACAGGCA
TTGGAGTTTTTACATGCTAATCAAGTGATCCACAGAGACATCAAAAGTGACAATGTACTTTTGGGAATGGAAG
GATCTGTTAAGCTCACTGACTTTGGTTTCTGTGCCCAGATCACCCCTGAGCAGAGCAAACGCAGTACCATGGT
CGGAACGCCATACTGGATGGCACCAGAGGTGGTTACACGGAAAGCTTATGGCCCTAAAGTCGACATATGGTCT
CTGGGTATCATGGCTATTGAGATGGTAGAAGGAGAGCCTCCATACCTCAATGAAAATCCCTTGAGGGCCTTGT
ACCTAATAGCAACTAATGGAACCCCAGAACTTCAGAATCCAGAGAAACTTTCCCCAATATTTCGGGATTTCTT
AAATCGATGTTTGGAAATGGATGTGGAAAAAAGGGGTTCAGCCAAAGAATTATTACAGCATCCTTTCCTGAAA
CTGGCCAAACCGTTATCTAGCTTGACACCACTGATCATGGCAGCTAAAGAAGCAATGAAGAGTAACCGTTAAC
ATCACTGCTGTGGCCTCATACTCTTTTTTCCATTTTCTACAAGAAGCCTTTTAGTATATGAAAATTATTACTC
TTTTTGGGGTTTAAAGAAATGGTCTGCATAACCTGAATGAAAGAAGCAAATGACTATTCTCTGAAGACAACCA
AGAGAAAATTGCAAAAAGACAAGTATGACTTTTATATGAACCCCTTCTTTAGGGTCCAGAAGGAATTGTGGAC
TGAATCACTAGCCTTAGGTCTTTCAGCAAACAGCCTATCAGGGCCATTTATCATGTGTGAGATTTGCATTTTA
CTTTGCTGACTTTGTTGTAATAGATCCCATTCATTGTCCCCTTTGGGGTATTTCCAATACTTGAATGGCAGAT
TGGAGTTTTTCAGAGTATGTGTTTCATCTGCTAGTCTTTCTCTCCTTCATAGCTTTTCTTTTCCTGGACTTGC
TCCTTTTGAGTTGCTTTTGCGTTTCTCATGCCTAGGCAAGTGTAATAGAAATTATGTAGCTCCTTATGTTGGC
AAAGGAGCTCTATATAGTTTCACTTTGTATAAAAGTTAGGACCAGCTGTTGTTACATGTAATATTTTAGTTCA
GAACTTGACCTGAAGGAAGGGAAGAAAAGTATGTGATTTTTACCTTTTTTAACAAATGTGAAAAAGTCAGTTT
TAGAAATTTCGTGGTAGTAAGTTCGGCATTTGTTACATGTATAGAGAGAAGACTAATAATCTCTATTTATAAC
TAAATCATTGAGATAGAAAAAGATTCCCATTGACTGTAGACTTCTTCCCATTTTGTCTTCCCTTCTGCCTGTT
TCCCCTTCAGGCTTGGCTCTAGGAACCAAAGTGATTTGTTGTTGTTCCAACCTGGGCTTTGTGACTTTGGTTA
GTGCCACTACCTTCTTCCCTCCTTTCCCCCTTCAATTTGGAAATAAATTTCTGTATATGTTGCAATTTTAGGT
TTAGGTTTGTTCTTTTTCTTTTTCATTAATCCTCTCTCACCTCACAGATACCCCCTCCCATGGCAAATAATAT
AATAACCAGTGAATTTTCAGGAATTTAAAAATTAGCTTTTTTCCACTTAAAGGAGAAAAATATTTGGGACTAG
CAGCAGAGGCAGTAAGAGATGTGAACCTTGGTGAGCTCTGATACAGTGAGAAGAGATTATACTCATGAAAGAG
AATGTTAGTGTTACAGAGAAGCAGCCGATAGCAAATCGACTGTAGAGACTTGGCGGCGGTGGCATTGCCCCAG
GTCGTCAGCAGTGTGGTATTATCTATGAGAACTTGAGCGACAGAGTATTTCTTGATGAATTTATAGATCATTT
GAGATGTTGAGTTACTTTAGTTTAGTTTTGTTTTGTTTTTTCAAATAAGTAGAGACTATTGTAAAAAACGAGA
AAGGAAAATGAAATGTGCGTGTTGATAGCAATAATTTGTTTCTTTTAAAGATTCTAAAAGGTCTGAGACCTGT
AGCATTAATTATTTGAGTGCCCTCCCTTCTCCCCTCCCCTCCCTTTTCTCTTCTCTTTTTTCCTCTCCTCTTC
TTCTCCTTTATTCATTGTTTTGCTTTTGGAGTGGGTGTTGTTCAAGTATCTGTGGTTTGGTTCTGGCATTTTG
TTCCCACCATCCCCTTCCCCCATTAACTTCCCCCCTGCTTGCCATCCTGCAGTAGTATAAATCATGAATAAAA
AATAATTTTGCTGTTGTAGTATACATTGGAGAAACTGGCAGGTTTTATTTCCATTATTTTATTTCCACTATAT
CTATGATAAGATGCAATTATAAGGAGAGAAGTGACTGTTTTTTATTGATAAGGCAAGATTTTCAGAAAAATGA
GTAAAATAATTAATGAAACATATTTAGAGCACTTAATGGTCTCTGTTTTCAATATAATTCTTGATTTCATTTT
TCTCTGGAATATATTGGCCTTCTACAGCTATTACTGAATTATAGAAACTGGTTTATTTCTGGCAGAAAGCTGC
AGTGCCACCTGAGTTCCAAATTTTACCATTCTTTGTAAACAGTTGGATGGATTATGATAAAGAAGATGCTACC
AATGAAATAGAAAACCAACGAGATGAGAAGACTGTGATCCTCATGTACTCAGAGGCACTTCCCTCCTAAGTCA
AAGACCATCCTCACTGACTATGTGCCAACGCCTCGTTTCAGGCTTGTGACTCAACAAAGGGCTTTTCCATTGA
TAGAAGCAGTTTGGGATTTGTAGTTGCGACTTCTTCGATAGTTACCTGCACGTCCATTGCTGGCAACTGACTT
GTCATTAAAACCTGGCTCTTTGGTTAAGGGAGCTACGCTGTGGTTTATTCTTAAGTTACGTGGATAAACTAAC
CTCTAACAGAAATATACTTTGGTTAATTTTGAAATGTGTCATTTTTAAACAATCTTAAAAGTAATACAGAATT
GTGATTTATTAATTTTAAAACATTCAGAACTTGTTGAAAGAAAAATTATATCTGAATCAAGATTCATGTTTTT
TATTTTTATTTTTTTTGATACAGAGTCTCACTCTGTCACTCAGGCTGGAGTGCAGTGACATGATCTCAGCTCA
CTGCAACCTCCGCTTCCTGGGTTCAAGCAATTCTCATGCCTCAGCCTCCTGAGTAGCTGAGACCACAGGCACC
CGCCACCACACCCAGCTATTTTTTTGTATTTTTAGTAGAGACAGAGTCTCACCACATTGCCCAGGCTGGTCTC
CAACTCCTGAGCTCAGGCAGTCTGCCCACCTTGGCCTCCCAAAGTGCTGCAGTTACAGGCGTGAGCCACTGCA
CCTGGCCTCATGTTTTTTAAATAATTGCCTTTTATATTTACCCTTTTTGTCATCACTTTAGAATGAAAATTCC
CATTTAAATCTGAAAGTTACCTTAATAGTCCTCTTGTGTTATTAGGACAGTATTATTATAGTACTTATTTATT
TTATTTTAGATTTAAAGTTATCTTCTCTTTTTCTTTTTTCTTCTGCTGCTTTTAGGGACAATTAAAACTGGGA
AACTATGAAACATGGAACATTTTATCCTACCTGAAAGTAAACGAGTAATTGTGAAGCATAAGACACTGAGGCT
AATACAACTCTGTCTTCATGTGTTGACTGCCTGGCACATAGTATTCATTCTCTTCCCTTTAACATAGAAGTGT
CCAGCTGCGTACAGTCTAGTAACCAGCAACTGTAAACGAACCTGTGCCTCTAACAAGCGATTCTAAACCACCT
ATGAGTATTTCTTTTAGGGCTCACTTAAATACATGTTTGTATATACTGTATTCTAGCCAGAATAATTTTAGAT
CTGATCAGGTAGTAGCTAAAATTAGAAAAAAACAAAATAGATGCTTAAAGAATTTGCATCCATTTTTGAGTCT
AAATCTTTTAAAATATACTGAGATCCACATCTAGTGAAATGTCAGTGTCAAAATATTATAGATTATAGCTAAA
ATCCAGATTAATACTCATTTGGGGTTTTTTATAGTGGAACTTCATAGTAATACAAAAAGCAGATTGTCTTCCT
GTCTCCGCTGCTCCCACAGTAGGTATTGAAACTGGTAAAATCAGTTTTTTGATAGTGTGTGTATATAAGAAAA
AATAGATACACACATTCTTTTTTCTCAGTCAACACATTGATTGAACACTCTGGCAAAGATGCTGTGGTGGATG
AGGTTGGAGTTCGAAAGAAGAAGCAAGCGCTGGCCTGGCCTTGAAAGAACCGAAGTCTTTCCCATTCACTTCT
CTAGAAAGCTGCCAAGACAGAGGCAGAAAGAAATGGATGATAGTTCTGTCAAGCACACTTCTGTTCTCTTAGA
ACTTAGAAGTGTTTCTAAGAGAACAGAAGTAATAAGAGAAACAGTTACGTGTGGAATTCAACATCTTTGGTTG
GAACGCATTGGCTTTTTTTTTCTTGTTTTGATAGAAATGGAATTAAGCAAAAGTAGTTTTTGTCTTTTCTGTT
GTCTTCAAATTTTATGCCTTTTATTTTTAATTTAATCCCGTTCAATTATTTAATTGTTATACATTGACATTAA
CTGCTGTATTTTGACTTTGTTCAATAATTTTGTTCTTTCAGGGCTAGAAATAAACTTTTTAAAAAAAGTGTGC
ATTTTTCCCTTTCCTAAACTTTTATTCTTTCTTTTGATCAGCGTAAAAGAATATTTTAATGTCTTTTGATAGC
ATAAAAGAATATTTAAATGTCTTAATAGGTTTTCAAAGAACATTTAGTATTTTTAGTGATAAATGTTTTAAAC
CTTTTAA
(SEQ ID NO: 177)
MSDNGELEDKPPAPPVRMSSTIFSTGGKDPLSANHSLKPLPSVPEEKKPRHKIISIFSGTEKGSKKKEKERPE
ISPPSDFEHTIHVGFDAVTGEFTGMPEQWARLLQTSNITKLEQKKNPQAVLDVLKFYDSNTVKQKYLSFTPPE
KDGFPSGTPALNAKGTEAPAVVTEEEDDDEETAPPVIAPRPDHTKSIYTRSVIDPVPAPVGDSHVDGAAKSLD
KQKKKTKMTDEEIMEKLRTIVSIGDPKKKYTRYEKIGQGASGTVFTATDVALGQEVAIKQINLQKQPKKELII
NEILVMKELKNPNIVNFLDSYLVGDELFVVMEYLAGGSLTDVVTETCMDEAQIAAVCRECLQALEFLHANQVI
HRDIKSDNVLLGMEGSVKLTDFGFCAQITPEQSKRSTMVGTPYWMAPEVVTRKAYGPKVDIWSLGIMAIEMVE
GEPPYLNENPLRALYLIATNGTPELQNPEKLSPIFRDELNRCLEMDVEKRGSAKELLQHPFLKLAKPLSSLTP
LIMAAKEAMKSNR*

An amino acid sequence for the protein encoded by the PDCL gene that is a regulator of T cell exhaustion/dysfunction resistance is available from the UniPROT database at accession no. Q13371. cDNA NM_005388, with gene location at Chr 9: 122798389-122828588.

```
  (SEQ ID NO: 178)
GGCTTTTCTCTTTAGCGCTCAGGGCGCTGACCCACTGCTCTTCCTCTTAAGAAAGTGCTCCATTCCTTCCGGC
GCCCGGAGCTGCTGGCCCAAAGGGATCCGGAGCGAGCTAGGGCAGACACCATGACCACCCTTGATGATAAGTT
GCTGGGGGAGAAACTGCAGTACTACTATAGCAGCAGTGAGGATGAGGACAGTGACCACGAGGACAAGGACCGA
GGCAGATGTGCCCCAGCCAGCAGTTCTGTGCCTGCAGAGGCTGAGCTGGCAGGCGAAGGCATCTCAGTTAACA
CAGGCCCAAAAGGTGTGATCAATGACTGGCGCCGCTTCAAGCAGTTGGAGACAGAGCAGAGGGAGGAGCAGTG
CCGGGAGATGGAAAGGCTGATCAAGAAGCTGTCAATGACTTGCAGGTCCCATCTGGATGAAGAGGAGGAGCAA
CAGAAACAGAAAGACCTCCAGGAGAAGATCAGTGGGAAGATGACTCTGAAGGAGTTTGCCATAATGAATGAGG
ACCAAGATGATGAAGAGTTTCTGCAGCAGTACCGGAAGCAGCGAATGGAAGAGATGCGGCAGCAGCTTCACAA
GGGGCCCCAATTCAAGCAGGTTTTTGAGATCTCCAGTGGAGAAGGGTTTTTAGACATGATTGATAAAGAACAG
AAAAGCATTGTCATCATGGTTCATATTTATGAGGATGGCATTCCAGGGACCGAAGCCATGAATGGTTGCATGA
TCTGCCTTGCCGCAGAGTACCCAGCTGTCAAGTTCTGCAAGGTGAAGAGCTCAGTTATTGGCGCCAGCAGTCA
GTTCACCAGGAATGCCCTTCCTGCCCTGCTGATCTATAAGGGGGGTGAATTGATCGGCAATTTTGTTCGTGTT
ACTGACCAGCTGGGGGATGATTTCTTTGCTGTGGACCTTGAAGCTTTTCTCCAGGAATTTGGATTACTCCCAG
AAAAGGAAGTCTTGGTGCTGACATCTGTGCGTAACTCTGCCACGTGTCACAGTGAGGATAGCGACCTGGAAAT
AGATTGAACTGATAGTCTAGTTGCATAGATTTCTCATTGTTTGGGTTGGAATACACGTCATTGTTTATTTTTG
TTCCTTTGTCTTCTGGCTTTTCAGCTGTTCTTTGTAGTCCCTTTTATTATGCATAAAATAAAGAAATTCTTAG
ATTAAATCAGAATGCTGAATAACCTTGTAGCTAGCAATAAGGTGACTTACAGTTGTATAAACAGGAAGCCAGG
CTTTTGAACTGTTTACTTAAGATTCTGTGGTGTGACATCTCTGTTATTGTTTCCAGTCAATATTTACAAAGCA
TCCTAAAGACAGGGTCTTGGAAATTGTCTTCAGATGATCTTAGAGGTCTCTGCCAAGTCTGAGAGTATAATTC
TGTAGGTATTGTGTTATTTGCAACGTAAATAGTGCATTTTCTTAATCAAATGATTGTAAATTATATTTACTTG
TAATCAGTTCCATAGCTTTAGACGGTGGTTAGATTTTTTTTTTCCCCACCAGGGTCTTGTTTAAAGGGGTGAG
CCACCGCACCCAGTCCTGAGGGGTGGCCTCTGCTGCTGGATTTCATGTCTTCCTCCAGCATGACTAAGTCTGG
AACAGCAGGAAGGGTTGATGCTTACTGACCTGGTGATGTTAGAAGACAAGTAGTTTATGGATTTAAACATTAG
AGCTGGAGTGGGGCTGGAAATCTTTGTAAAGGAAGTTCTTTCAGTAAGATGCCCCTGCTTGTCTTTGTCTCTT
TTTTGTTTAACAAGGTAACTTTTTGTTTAACAAGGTAACTTTTTGTTTAACCTAGATTTTTTTTAAAACTTTT
TTTTTTTTTCATATTGGAAAAGTAATTCATATTCAGTAGAGGAAAACTGACCAAAACAGAAGCAAAAATAAGA
AAATTAAAATAATCTCTAATCCTACTACCTAGAATAAAACACTATTAATATTTTGGTCTGTTTCCTGCCAAGG
TGTTTTCTGTGTATACATGGATATTTTGTTTGTTTTTAAACAAAACGATGGGATCATTCTGAACATACTGTTC
TATAGTATGGTCAGCTAATAATATATCAGACCTTTTTTTATATTATTAAATATTCTACAACTTTTTAAAAATG
TCTATTAATATTCCATCGTATAGATGTGATATAATTTGCTTGATGGTTGTCTCTTAAAAAGAAAGATAGCAAA
TACTTTTTTTAAATTACAAAAGTGATAGATGTTCATTGTAGAAAATGTAATAAACACTGTTAAGACTTAAAAG
CCATATAATTCCACCAACCAAAATTAATCCCTTTTGTCATATTTCTAGTCATTTTTATAGCCTTTTTTTCTAT
GTATTTATAATAATTATCATTTGCGTTTTTTTCCTTTTTTTAACTTTAAAAATGTATATTCTAGGGTCAGGGG
AAATGTAATCTGGAATTAAATATTAGCCTTAAAATTCACAATTTTGATTTTCCTGGCTTTTCAGGAATTGACT
AACTGTAAAAGAGTCTTGAAAGTATTTAGTCAACAAACAGAGTGCATTTTTTTTTTTTTTGACTAAGAAAGCT
CGTTGTAGTAGAAAGGGTGGAATGTATTGAAAATTATTAGAAGCAGGGAAGTATTGTTAGTCTAGCTTATTTC
CTTTCAGTCTTTTTTCAATATTTTTATAAACATTGAGTACTTACTGAATTTAGTTCTGTGCTCTTCCTTATTT
AGTGTTGTATCATAAATACTTTGATGTTTCAAACATTCTAAATAAATAATTTTCAGTGGCTTCATAATATTTC
ATCATTTGGATATATAATAATTTATTTAACCAGTCTCTTTATTTTTATTTATTTATTTTGTTTGTTTTCTTTG
AGATGGAGTCTCGCTCTGTCACCCAGGCTGGAGTACAATAGCGTGATCTCGGCTCACTGCAACCTCCACCTCC
TGGGTTCAAGCAGTTCTCCTGCCTCAGCCTCCTGAGTAGCTGGGACTACAGGCACATGCCATCACGCCTGGCT
AATTTTTGTATTTTTACTAGAGAC
  (SEQ ID NO: 179)
MTTLDDKLLGEKLQYYYSSSEDEDSDHEDKDRGRCAPASSSVPAEAELAGEGISVNTGPKGVINDWRRFKOLE
TEQREEQCREMERLIKKLSMTCRSHLDEEEEQQKQKDLQEKISGKMTLKEFAIMNEDQDDEEFLQQYRKORME
EMRQQLHKGPQFKQVFEISSGEGFLDMIDKEQKSIVIMVHIYEDGIPGTEAMNGCMICLAAEYPAVKFCKVKS
SVIGASSQFTRNALPALLIYKGGELIGNFVRVTDQLGDDFFAVDLEAFLQEFGLLPEKEVLVLTSVRNSATCH
SEDSDLEID*
```

An amino acid sequence for the protein encoded by the PIK3R5 gene that is a regulator of T cell exhaustion/dysfunction resistance is available from the UniPROT database at accession no. Q8WYR1, cDNA NM_001142633, with gene location at Chr 17: 8878911-8965712.

```
  (SEQ ID NO: 180)
AGATGCTGCGGCTCGCCGCGCGGCTCCAGCTGGGGCGCGGTGGTCCCGACGGTCACCCGAATCGCAGGGAGTC
GCCGCTGGAGAGGGGCTCGGCGCCCGGCGGGGACTGCAGGCGATGACCCAGGATGCAGCCAGGGGCCACGACA
TGCACGGAGGACCGCATCCAGCATGCCCTGGAACGCTGCCTGCATGGACTCAGCCTCAGCCGCCGCTCCACCT
CCTGGTCAGCTGGGCTGTGTCTGAACTGCTGGAGCCTGCAGGAGCTGGTCAGCAGGGACCCGGGCCACTTCCT
TATCCTCCTTGAGCAGATCCTGCAGAAGACCCGAGAGGTCCAGGAGAAGGGCACCTACGACCTGCTCACCCCG
CTGGCCCTGCTCTTCTATTCCACTGTTCTTTGTACACCACACTTCCCACCAGACTCGGATCTCCTTCTGAAGG
CAGCCAGCACCTACCACCGGTTCCTGACCTGGCCTGTTCCTTACTGCAGCATCTGCCAGGAGCTGCTCACCTT
CATTGATGCTGAACTCAAGGCCCCAGGGATCTCCTACCAGAGACTGGTGAGGGCTGAGCAGGGCCTGCCCATC
AGGAGTCACCGCAGCTCCACCGTCACCGTGCTGCTGCTGAACCCAGTGGAAGTGCAGGCCGAGTTCCTTGCTG
TAGCCAATAAGCTGAGTACGCCCGGACACTCGCCTCACAGTGCCTACACCACCCTGCTCCTGCACGCCTTCCA
GGCCACCTTTGGGGCCCACTGTGACGTCCCGGGCCTGCACTGCAGGCTACAGGCCAAGACCCTGGCAGAGCTT
GAGGACATCTTCACGGAGACCGCAGAGGCACAGGAGCTGGCATCTGGCATCGGGGATGCTGCAGAGGCCCGGC
GGTGGCTCAGGACCAAGCTGCAGGCGGTGGGAGAAAAAGCTGGCTTCCCTGGGGTGTTAGACACTGCAAAACC
AGGGAAGCTCCACACCATCCCCATCCCTGTCGCCAGGTGCTACACCTACAGCTGGAGCCAGGACAGCTTTGAC
ATCCTGCAGGAAATCCTGCTCAAGGAACAGGAGCTACTCCAGCCAGGGATCCTGGGAGATGATGAAGAGGAGG
AAGAGGAGGAGGAGGAGGTGGAGGAGGACTTGGAAACTGACGGGCACTGTGCCGAGAGAGATTCCCTGCTCTC
CACCAGCTCTTTGGCGTCCCATGACTCCACCTTGTCCCTTGCATCCTCCCAGGCCTCGGGGCCGGCCCTCTCG
CGCCATCTGCTGACTTCCTTTGTCTCAGGCCTCTCTGATGGCATGGACAGCGGCTACGTGGAGGACAGCGAGG
AGAGCTCCTCCGAGTGGCCTTGGAGGCGTGGCAGCCAGGAACGCCGAGGCCACCGCAGGCCTGGGCAGAAGTT
CATCAGGATCTATAAACTCTTCAAGAGCACCAGCCAGCTGGTACTGCGGAGGGACTCTCGGAGCCTGGAGGGC
AGCTCGGACACGGCCCTGCCCCTGAGGCGGGCAGGGAGCCTCTGCAGCCCCCTGGACGAACCAGTATCACCCC
CTTCCCGGGCCCAGCGCTCCCGCTCCCTGCCCCAGCCCAAACTCGGTACCCAGCTGCCCAGCTGGCTTCTGGC
CCCTGCTTCACGCCCCCAGCGCCGCCGCCCCTTCCTGAGTGGAGATGAGGATCCCAAGGCTTCCACGCTACGT
GTTGTGGTCTTTGGCTCCGATCGGATTTCAGGGAAGGTGGCTCGGGCGTACAGCAACCTTCGGCGGCTGGAGA
ACAATCGCCCACTCCTCACACGGTTCTTCAAACTTCAGTTCTTCTACGTGCCTGTGAAGCGAAGTCATGGGAC
CAGCCCTGGTGCCTGTCCACCCCCTCGGAGCCAGACGCCCTCACCCCCGACAGACTCCCCTAGGCACGCCAGC
CCTGGAGAGCTGGGCACCACCCCATGGGAGGAGAGCACCAATGACATCTCCCACTACCTCGGCATGCTGGACC
CCTGGTATGAGCGCAATGTACTGGGCCTCATGCACCTGCCCCCTGAAGTCCTGTGCCAGCAGTCCCTGAAGGC
TGAAGCCCAGGCCCTGGAGGGCTCCCCAACCCAGCTGCCCATCCTGGCTGACATGCTACTCTACTACTGCCGC
TTTGCCGCCAGACCGGTGCTGCTGCAAGTCTATCAGACCGAGCTGACCTTCATCACTGGGGAGAAGACGACAG
AGATCTTCATCCACTCCTTGGAGCTGGGTCACTCCGCTGCCACACGTGCCATCAAGGCGTCAGGTCCTGGCAG
CAAGCGGCTGGGCATCGATGGCGACCGGGAGGCTGTTCCTCTAACACTACAGATTATTTACAGCAAGGGGGCC
ATCAGTGGACGAAGTCGCTGGAGCAACCTGGAGAAGGTCTGTACCTCCGTGAACCTCAACAAGGCCTGCCGGA
AGCAGGAGGAGCTGGATTCCAGCATGGAGGCCCTGACGCTAAACCTGACAGAAGTGGTGAAAAGGCAGAACTC
CAAATCCAAGAAGGGCTTTAACCAGATTAGCACATCGCAGATCAAAGTGGACAAGGTGCAGATCATCGGCTCC
AACAGCTGCCCCTTTGCTGTGTGCCTGGACCAGGATGAGAGAAAGATCCTGCAGAGTGTAGTCAGATGTGAGG
TCTCACCGTGCTACAAGCCAGAGAAGAGCGACCTCTCCTCACCACCCCAGACGCCTCCTGACCTGCCGGCCCA
GGCCGCACCTGATCTCTGCTCCCTTCTCTGCCTGCCCATCATGACTTTCAGTGGAGCTCTGCCCTAGTGTGGG
CCCAGCGCCAGACTGGACAGAAGCCCTGGGGCAACCTCCTCGGCCACCCCTCCAGGACAGTCCCTCTCTGTGG
AGAACTGAATGGCCCTGTGCAGAGCCATAGTCCCACTGTGGGTCCTGCAATGAGCAGGGGCTGGGAGTAGAGG
GTTTCTGGGGCCTCAGGGTTCTGGGAAAGCAACAGCTATCAGAGAGAGAAGGGCCAGACCCCATAGCCTCTTA
GATTCCTGGCAGTAGAAGGAGAAGGATGGGTAAATTGACCTCTGAAGTCCCTGACCATTAGCATGGTCTAGGA
TCCTTTCTAGAAGGAAGATCTGAGGCTCTGGTGCTCAGGGGGATGGCTTGGGCCTTTTCTCTCAACCTTGGCT
GAGCCTACCCCTTACTTTGCCAAAGACTTGAGGACCCTGTATGTCTGGAGTTCAGTCCCCTCCTCTGTGGGGC
TCAGGTGATTGAAATGTGGATGAAACATTTCTCTACTTCAAGACCACCTCTCCCTGCAAACACCACACACACA
TGGCATGCATGTACGCACATGCGCACACACACACGCACACACCTCAATAATTTCTCTCAAGTTTCCTGAGTCT
CCAGAAAAACAGCACTAACGCTGGACCTGTCTACTCTCAGAACCCGGCACAGATTCTCTCTTGATCTCCTTTT
GGAATCTGAGATTCTTAGAAGACAGGATAGGGTTAAATTTAGTAGCAGCTCAGTTCTAGCTAAATCACTAGAG
GAAGTTAATTAACTTTAAGCCTTCATTTCTCCAGCACTAAAATGGAGTGGAGAGTTGGGGTGGAAATAAGACA
TCCTTAAAAGGTTAAATTGTCTGCAAAGCACCTAGCCCAGTGCCGAGCTCCCAGTAGGTGTTCAGTAAAGCTT
AGTGCCTGACTTTCTGAACACTGATTCCTCCTGTTTGGAGTCACTGGGATACTCTCATTGCCGTTGGGATGTT
CCTCACTCCTTCCCAGTTCGTGGCTGAGGCAGAACCCAGACTGAAGAGGGAAGAGACATTCCAGAGGAGGATT
GCCTTCGTCAGGGTAAGGGGTGGGCTGCTCAGGGGCCCTACCCTTCACCCCCTTCTGTATCAGATTGGCCCTC
CCACTCCCATCTCACTCTGCGTGTACAATCTTCCATATCCGCAAGTTCACTGGCACTCTTCTGGCACCTGGGC
AAGATCCCAGAACAGAGGATGGAGTGACTGGCCTCACAGAGCTTAGTGCCCGACTCAGGGGAAATGGGACTGG
TGCATGGGAAATGGTCAGCCTAGGATAGGACACGAGAGTCTGAAATTCAAAGCAACCAGCTTGAAGTGGTTTG
AGAAGCTGGAAGCAAACATGGGCTAGAGAGATAGGGCAGAAGTCAAGACGAGGATCTGGACTGATGTGGAGAA
AGTAGCCACGGAAGCATGAACTGTATCCTGCACAAAGTCCCTCTTCCCCGCCTCCTAATTCATTATGCCCAAA
AGGCCTTACGTGAAATTCCAGCCCAGAGTACTCATGACTTGAGAGACGTGGACAGAGCCAGCTTCTACCTTGC
CTGGCCGTCTCTCCCCTGTCTTAATGTCTGCTCTTGCTCTAAGCTCCAGAAGAGTGGCGGGCCATGTATCTTC
AATATGTTTTTGCTGTATGGGCAGGTTGTCTTATTATGTGATCAACAGATGTCCAGGAACTAATGAGTGGAAT
TTAATATTATTGTCAAATAAAACTTGATTTGTCCTATA
  (SEQ ID NO: 181)
MQPGATTCTEDRIQHALERCLHGLSLSRRSTSWSAGLCLNCWSLQELVSRDPGHFLILLEQILQKTREVQEKG
TYDLLTPLALLFYSTVLCTPHFPPDSDLLLKAASTYHRFLTWPVPYCSICQELLTFIDAELKAPGISYQRLVR
AEQGLPIRSHRSSTVTVLLLNPVEVQAEFLAVANKLSTPGHSPHSAYTTLLLHAFQATFGAHCDVPGLHCRLQ
AKTLAELEDIFTETAEAQELASGIGDAAEARRWLRTKLQAVGEKAGFPGVLDTAKPGKLHTIPIPVARCYTYS
WSQDSFDILQEILLKEQELLQPGILGDDEEEEEEEEEVEEDLETDGHCAERDSLLSTSSLASHDSTLSLASSQ
ASGPALSRHLLTSFVSGLSDGMDSGYVEDSEESSSEWPWRRGSQERRGHRRPGQKFIRIYKLFKSTSQLVLRR
DSRSLEGSSDTALPLRRAGSLCSPLDEPVSPPSRAQRSRSLPQPKLGTQLPSWLLAPASRPQRRRPFLSGDED
PKASTLRVVVEGSDRISGKVARAYSNLRRLENNRPLLTRFFKLQFFYVPVKRSHGTSPGACPPPRSQTPSPPT
DSPRHASPGELGTTPWEESTNDISHYLGMLDPWYERNVLGLMHLPPEVLCQQSLKAEAQALEGSPTQLPILAD
MLLYYCRFAARPVLLQVYQTELTFITGEKTTEIFIHSLELGHSAATRAIKASGPGSKRLGIDGDREAVPLTLQ
IIYSKGAISGRSRWSNLEKVCTSVNLNKACRKQEELDSSMEALTLNLTEVVKRQNSKSKKGFNQISTSQIKVD
KVQIIGSNSCPFAVCLDQDERKILQSVVRCEVSPCYKPEKSDLSSPPQTPPDLPAQAAPDLCSLLCLPIMTES
GALP*
```

An amino acid sequence for the protein encoded by the PTEN gene that is a regulator of T cell exhaustion/dysfunction resistance is available from the UniPROT database at accession no. P60484, cDNA NM_000314, with gene location at Chr 10: 87862638-87971930.

```
  (SEQ ID NO: 182)
GTTCTCTCCTCTCGGAAGCTGCAGCCATGATGGAAGTTTGAGAGTTGAGCCGCTGTGAGGCGAGGCCGGGCTC
AGGCGAGGGAGATGAGAGACGGCGGCGGCCGCGGCCCGGAGCCCCTCTCAGCGCCTGTGAGCAGCCGCGGGGG
CAGCGCCCTCGGGGAGCCGGCCGGCCTGCGGCGGCGGCAGCGGCGGCGTTTCTCGCCTCCTCTTCGTCTTTTC
TAACCGTGCAGCCTCTTCCTCGGCTTCTCCTGAAAGGGAAGGTGGAAGCCGTGGGCTCGGGCGGGAGCCGGCT
GAGGCGCGGCGGCGGCGGCGGCACCTCCCGCTCCTGGAGCGGGGGGGAGAAGCGGCGGCGGCGGCGGCCGCGG
CGGCTGCAGCTCCAGGGAGGGGGTCTGAGTCGCCTGTCACCATTTCCAGGGCTGGGAACGCCGGAGAGTTGGT
CTCTCCCCTTCTACTGCCTCCAACACGGCGGCGGCGGCGGCTGGCACATCCAGGGACCCGGGCCGGTTTTAAA
CCTCCCGTGCGCCGCCGCCGCACCCCCCGTGGCCCGGGCTCCGGAGGCCGCCGGCGGAGGCAGCCGTTCGGAG
GATTATTCGTCTTCTCCCCATTCCGCTGCCGCCGCTGCCAGGCCTCTGGCTGCTGAGGAGAAGCAGGCCCAGT
CGCTGCAACCATCCAGCAGCCGCCGCAGCAGCCATTACCCGGCTGCGGTCCAGAGCCAAGCGGCGGCAGAGCG
AGGGGCATCAGCTACCGCCAAGTCCAGAGCCATTTCCATCCTGCAGAAGAAGCCCCGCCACCAGCAGCTTCTG
CCATCTCTCTCCTCCTTTTTCTTCAGCCACAGGCTCCCAGACATGACAGCCATCATCAAAGAGATCGTTAGCA
GAAACAAAAGGAGATATCAAGAGGATGGATTCGACTTAGACTTGACCTATATTTATCCAAACATTATTGCTAT
GGGATTTCCTGCAGAAAGACTTGAAGGCGTATACAGGAACAATATTGATGATGTAGTAAGGTTTTTGGATTCA
AAGCATAAAAACCATTACAAGATATACAATCTTTGTGCTGAAAGACATTATGACACCGCCAAATTTAATTGCA
GAGTTGCACAATATCCTTTTGAAGACCATAACCCACCACAGCTAGAACTTATCAAACCCTTTTGTGAAGATCT
TGACCAATGGCTAAGTGAAGATGACAATCATGTTGCAGCAATTCACTGTAAAGCTGGAAAGGGACGAACTGGT
GTAATGATATGTGCATATTTATTACATCGGGGCAAATTTTTAAAGGCACAAGAGGCCCTAGATTTCTATGGGG
AAGTAAGGACCAGAGACAAAAAGGGAGTAACTATTCCCAGTCAGAGGCGCTATGTGTATTATTATAGCTACCT
GTTAAAGAATCATCTGGATTATAGACCAGTGGCACTGTTGTTTCACAAGATGATGTTTGAAACTATTCCAATG
TTCAGTGGCGGAACTTGCAATCCTCAGTTTGTGGTCTGCCAGCTAAAGGTGAAGATATATTCCTCCAATTCAG
GACCCACACGACGGGAAGACAAGTTCATGTACTTTGAGTTCCCTCAGCCGTTACCTGTGTGTGGTGATATCAA
AGTAGAGTTCTTCCACAAACAGAACAAGATGCTAAAAAAGGACAAAATGTTTCACTTTTGGGTAAATACATTC
TTCATACCAGGACCAGAGGAAACCTCAGAAAAAGTAGAAAATGGAAGTCTATGTGATCAAGAAATCGATAGCA
TTTGCAGTATAGAGCGTGCAGATAATGACAAGGAATATCTAGTACTTACTTTAACAAAAAATGATCTTGACAA
AGCAAATAAAGACAAAGCCAACCGATACTTTTCTCCAAATTTTAAGGTGAAGCTGTACTTCACAAAAACAGTA
GAGGAGCCGTCAAATCCAGAGGCTAGCAGTTCAACTTCTGTAACACCAGATGTTAGTGACAATGAACCTGATC
ATTATAGATATTCTGACACCACTGACTCTGATCCAGAGAATGAACCTTTTGATGAAGATCAGCATACACAAAT
TACAAAAGTCTGAATTTTTTTTTATCAAGAGGGATAAAACACCATGAAAATAAACTTGAATAAACTGAAAATG
GACCTTTTTTTTTTTAATGGCAATAGGACATTGTGTCAGATTACCAGTTATAGGAACAATTCTCTTTTCCTGA
CCAATCTTGTTTTACCCTATACATCCACAGGGTTTTGACACTTGTTGTCCAGTTGAAAAAAGGTTGTGTAGCT
GTGTCATGTATATACCTTTTTGTGTCAAAAGGACATTTAAAATTCAATTAGGATTAATAAAGATGGCACTTTC
CCGTTTTATTCCAGTTTTATAAAAAGTGGAGACAGACTGATGTGTATACGTAGGAATTTTTTCCTTTTGTGTT
CTGTCACCAACTGAAGTGGCTAAAGAGCTTTGTGATATACTGGTTCACATCCTACCCCTTTGCACTTGTGGCA
ACAGATAAGTTTGCAGTTGGCTAAGAGAGGTTTCCGAAGGGTTTTGCTACATTCTAATGCATGTATTCGGGTT
AGGGGAATGGAGGGAATGCTCAGAAAGGAAATAATTTTATGCTGGACTCTGGACCATATACCATCTCCAGCTA
TTTACACACACCTTTCTTTAGCATGCTACAGTTATTAATCTGGACATTCGAGGAATTGGCCGCTGTCACTGCT
TGTTGTTTGCGCATTTTTTTTTAAAGCATATTGGTGCTAGAAAAGGCAGCTAAAGGAAGTGAATCTGTATTGG
GGTACAGGAATGAACCTTCTGCAACATCTTAAGATCCACAAATGAAGGGATATAAAAATAATGTCATAGGTAA
GAAACACAGCAACAATGACTTAACCATATAAATGTGGAGGCTATCAACAAAGAATGGGCTTGAAACATTATAA
AAATTGACAATGATTTATTAAATATGTTTTCTCAATTGTAACGACTTCTCCATCTCCTGTGTAATCAAGGCCA
GTGCTAAAATTCAGATGCTGTTAGTACCTACATCAGTCAACAACTTACACTTATTTTACTAGTTTTCAATCAT
AATACCTGCTGTGGATGCTTCATGTGCTGCCTGCAAGCTTCTTTTTTCTCATTAAATATAAAATATTTTGTAA
TGCTGCACAGAAATTTTCAATTTGAGATTCTACAGTAAGCGTTTTTTTTCTTTGAAGATTTATGATGCACTTA
TTCAATAGCTGTCAGCCGTTCCACCCTTTTGACCTTACACATTCTATTACAATGAATTTTGCAGTTTTGCACA
TTTTTTAAATGTCATTAACTGTTAGGGAATTTTACTTGAATACTGAATACATATAATGTTTATATTAAAAAGG
ACATTTGTGTTAAAAAGGAAATTAGAGTTGCAGTAAACTTTCAATGCTGCACACAAAAAAAAGACATTTGATT
TTTCAGTAGAAATTGTCCTACATGTGCTTTATTGATTTGCTATTGAAAGAATAGGGTTTTTTTTTTTTTTTTT
TTTTTTTTTTTTAAATGTGCAGTGTTGAATCATTTCTTCATAGTGCTCCCCCGAGTTGGGACTAGGGCTTCAA
TTTCACTTCTTAAAAAAAATCATCATATATTTGATATGCCCAGACTGCATACGATTTTAAGCGGAGTACAACT
ACTATTGTAAAGCTAATGTGAAGATATTATTAAAAAGGTTTTTTTTTCCAGAAATTTGGTGTCTTCAAATTAT
ACCTTCACCTTGACATTTGAATATCCAGCCATTTTGTTTCTTAATGGTATAAAATTCCATTTTCAATAACTTA
TTGGTGCTGAAATTGTTCACTAGCTGTGGTCTGACCTAGTTAATTTACAAATACAGATTGAATAGGACCTACT
AGAGCAGCATTTATAGAGTTTGATGGCAAATAGATTAGGCAGAACTTCATCTAAAATATTCTTAGTAAATAAT
GTTGACACGTTTTCCATACCTTGTCAGTTTCATTCAACAATTTTTAAATTTTTAACAAAGCTCTTAGGATTTA
CACATTTATATTTAAACATTGATATATAGAGTATTGATTGATTGCTCATAAGTTAAATTGGTAAAGTTAGAGA
CAACTATTCTAACACCTCACCATTGAAATTTATATGCCACCTTGTCTTTCATAAAAGCTGAAAATTGTTACCT
AAAATGAAAATCAACTTCATGTTTTGAAGATAGTTATAAATATTGTTCTTTGTTACAATTTCGGGCACCGCAT
ATTAAAACGTAACTTTATTGTTCCAATATGTAACATGGAGGGCCAGGTCATAAATAATGACATTATAATGGGC
TTTTGCACTGTTATTATTTTTCCTTTGGAATGTGAAGGTCTGAATGAGGGTTTTGATTTTGAATGTTTCAATG
TTTTTGAGAAGCCTTGCTTACATTTTATGGTGTAGTCATTGGAAATGGAAAAATGGCATTATATATATTATAT
ATATAAATATATATTATACATACTCTCCTTACTTTATTTCAGTTACCATCCCCATAGAATTTGACAAGAATTG
CTATGACTGAAAGGTTTTCGAGTCCTAATTAAAACTTTATTTATGGCAGTATTCATAATTAGCCTGAAATGCA
TTCTGTAGGTAATCTCTGAGTTTCTGGAATATTTTCTTAGACTTTTTGGATGTGCAGCAGCTTACATGTCTGA
AGTTACTTGAAGGCATCACTTTTAAGAAAGCTTACAGTTGGGCCCTGTACCATCCCAAGTCCTTTGTAGCTCC
TCTTGAACATGTTTGCCATACTTTTAAAAGGGTAGTTGAATAAATAGCATCACCATTCTTTGCTGTGGCACAG
GTTATAAACTTAAGTGGAGTTTACCGGCAGCATCAAATGTTTCAGCTTTAAAAAATAAAAGTAGGGTACAAGT
TTAATGTTTAGTTCTAGAAATTTTGTGCAATATGTTCATAACGATGGCTGTGGTTGCCACAAAGTGCCTCGTT
TACCTTTAAATACTGTTAATGTGTCATGCATGCAGATGGAAGGGGTGGAACTGTGCACTAAAGTGGGGGCTTT
AACTGTAGTATTTGGCAGAGTTGCCTTCTACCTGCCAGTTCAAAAGTTCAACCTGTTTTCATATAGAATATAT
ATACTAAAAAATTTCAGTCTGTTAAACAGCCTTACTCTGATTCAGCCTCTTCAGATACTCTTGTGCTGTGCAG
CAGTGGCTCTGTGTGTAAATGCTATGCACTGAGGATACACAAAAATACCAATATGATGTGTACAGGATAATGC
CTCATCCCAATCAGATGTCCATTTGTTATTGTGTTTGTTAACAACCCTTTATCTCTTAGTGTTATAAACTCCA
CTTAAAACTGATTAAAGTCTCATTCTTGTCATTGTGTGGGTGTTTTATTAAATGAGAGTTTATAATTCAAATT
GCTTAAGTCCATTGAAGTTTTAATTAATGGGCAGCCAAATGTGAATACAAAGTTTTCAGTTTTTTTTTTTCCT
GCTGTCCTTCAAAGCCTACTGTTTAAAAAAAAAAAAAAAAAAAAACATGGCCTGAGAGTAGAGTATCTGTCTA
CTCATGTTTAATTAAGGAAAAACACTTATTTTTAGGGCTTTAGTCATCACTTCATAAATTGTATAAGCACATT
AAATAGCGTTCTAGTCCTGAAAAAGTCCAAGATTCTTAGAAAATTGTGCATATTTTTATTATGACAGATGTTT
GAAGATAATTCCCCAGAATGGATTTGATACTTTAGATTTCAATTTTGTGGCTTTTGTCTATTATTCTGTACTC
TGCCATCAGCATATGGAAAGCTTCATTTACTCATCATGACTTGTGCCATATAAAAATTGATATTTCGGAATAG
TCTAAAGGACTTTTTGTACTTGAATTTAATCATGTTGTTTCTAATATTCTTAAAAGCTTGAAGACTAAAGCAT
ATCCTTTCAACAAAGCATAGTAAGGTAATAAGAAAGTGTAGTTTGTACAAGTGTTAAAAAAATAAAGTAGACA
ATGTTACAGTGGGACTTATTATTTCAAGTTTACATTTTCTCCATGTAATTTTTTAAAAAGTAAATGAAAAAAT
GTGCAATAATGTAAAATATGAAGTGTATGTGTACACACATTTTATTTTTCGGTATCTTGGGTATACGTATGGT
TGAAAACTATACTGGAGTCTAAAAGTATTCTAATTTATAAGAAGACATTTTGGTGATGTTTGAAAAATAGAAA
TGTGCTAGTTTTGTTTTTATATCATGTCCTTTGTACGTTGTAATATGAGCTGGCTTGGTTCAGTAAATGCCAT
CACCATTTCCATTGAGAATTTAAAACTCACCAGTGTTTAATATGCAGGCTTCCAAAGGCTTATGAAAAAAATC
AAGACCCTTAAATCTAGTTAATTTGCTGCTAACATGAAACTCTTTGGTTCTTTTATTTTTGCCAGATAATTAG
ACACACATCTAAAGCTTAGTCTTAAATGGCTTAAGTGTAGCTATTGATTAGTGCTGTTGCTAGTTCAGAAAGA
AATGTTTGTGAATGGAAACAAGAATATTCAGTCCAAACTGTTGTAAGGACAGTACCTGAAAACCAGGAAACAG
GATAATGGAAAAAGTCTTTTAAAGATGAAATGTTGGAGCCAACTTTCTTATAGAATTAATTGTATGTGGCTAT
AGAAAGCCTAATGATTGTTGCTTATTTTTGAGAGCATATTATTCTTTTATGACCATAATCTTGCTGTTTTTCC
ATCTTCCAAAAGATCTTCCTTCTAATATGTATATCAGAATGTGGGTAGCCAGTCAGACAAATTCATATTGGTT
GGTAGCTTTAAAAAGTTTGTAATGTGAAGACAGGAAAGGACAAAATAGTTTGCTTTGGTGGTAGTACTCTGGT
TGTTAAGCTAGGTATTTTGAGACTACTTCCCCATCACAACAACAATAAAATAATCACTCATAATCCTATCACC
TGGAGACATAGCCATCGTTAATATGTTAGTGACTATACAATCATGTTTTCTTCTGTATATCCATGTATATTCT
TTAAAAATGAAATTTATACTGTACCTGATCTCAAAGCTTTTTAGCTTAGTATATCTGTCATGAATTTGTAGGA
TGTTCCATTGCATCAGAAAACGGACAGTGATTTGATTACTTTCTAATGCCACAGATGCAGATTACATGTAGTT
ATTGAGAATCCTTTCGAATTCAGTGGCTTAATCATGAATGTCTAAATATTGTTGACATTAGGATGATACATGT
AAATTAAAGTTACATTTGTTTAGCATAGACAAGCTTAACATTGTAGATGTTTCTCTTCAAAAATCATCTTAAA
CATTTGCATTTGGAATTGTGTTAAATAGAATGTGTGAAACACTGTATTAGTAAACTTCATCACCTTTCTACTT
CCTTATAGTTTGAACTTTTCAGTTTTTGTAGTTCCCAAACAGTTGCTCAATTTAGAGCAAATTAATTTAACAC
CTGCCAAAAAAAGGCTGCTGTTGGCTTATCAGTTGTCTTTAAATTCAAATGCTCATGTGACTTTTATCACATC
AAAAAATATTTCATTAATGATTCACCTTTAGCTCTGAAAATTACCGCGTTTAGTAATTATAGTGGGCTTATAA
AAACATGCAACTCTTTTTGATAGTTATTTGAGAATTTTGGTGAAAAATATTTAGCTGAGGGCAGTATAGAACT
TATAAACCAATATATTGATATTTTTAAAACATTTTTACATATAAGTAAACTGCCATCTTTGAGCATAACTACA
TTTAAAAATAAAGCTGCATATTTTTAAATCAAGTGTTTAACAAGAATTTATATTTTTTATTTTTTAAAATTAA
AAATAATTTATATTTCCTCTGTTGCATGAGGATTCTCATCTGTGCTTATAATGGTTAGAGATTTTATTTGTGT
GGAATGAAGTGAGGCTTGTAGTCATGGTTCTAGTGTTTCAGTTTGCCAAGTCTGTTTACTGCAGTGAAATTCA
TCAAATGTTTCAGTGTGGTTTTCTGTAGCCTATCATTTACTGGCTATTTTTTTATGTACACCTTTAGGATTTT
CTGCCTACTCTATCCAGTTGTCCAAATGATATCCTACATTTTACAAATGCCCTTTCAGTTTCTATTTTCTTTT
TCCATTAAATTGCCCTCATGTCCTAATGTGCAGTTTGTAAGTGTGTGTGTGTGTGTCTGTGTGTGTGTGAATT
TGATTTTCAAGAGTGCTAGACTTCCAATTTGAGAGATTAAATAATTTAATTCAGGCAAACATTTTTCATTGGA
ATTTCACAGTTCATTGTAATGAAAATGTTAATCCTGGATGACCTTTGACATACAGTAATGAATCTTGGATATT
AATGAATTTGTTAGTAGCATCTTGATGTGTGTTTTAATGAGTTATTTTCAAAGTTGTGCATTAAACCAAAGTT
GGCATACTGGAAGTGTTTATATCAAGTTCCATTTGGCTACTGATGGACAAAAAATAGAAATGCCTTCCTATGG
AGAGTATTTTTCCTTTAAAAAATTAAAAAGGTTAATTATTTTGACTA
  (SEQ ID NO: 183)
MTAIIKEIVSRNKRRYQEDGEDLDLTYIYPNIIAMGFPAERLEGVYRNNIDDVVRFLDSKHKNHYKIYNLCAE
RHYDTAKFNCRVAQYPFEDHNPPQLELIKPFCEDLDQWLSEDDNHVAAIHCKAGKGRTGVMICAYLLHRGKEL
KAQEALDFYGEVRTRDKKGVTIPSQRRYVYYYSYLLKNHLDYRPVALLFHKMMFETIPMFSGGTCNPQFVVCQ
LKVKIYSSNSGPTRREDKFMYFEFPQPLPVCGDIKVEFFHKQNKMLKKDKMFHFWVNTFFIPGPEETSEKVEN
GSLCDQEIDSICSIERADNDKEYLVLTLTKNDLDKANKDKANRYFSPNFKVKLYFTKTVEEPSNPEASSSTSV
TPDVSDNEPDHYRYSDTTDSDPENEPFDEDQHTQITKV*
```

An amino acid sequence for the protein encoded by the RASA2 gene that is a regulator of T cell exhaustion/dysfunction resistance is available from the UniPROT database at accession no. Q15283, cDNA NM_001303245, with gene location at Chr 3: 141487027-141615344.

(SEQ ID NO: 184)
GCTCCGCCTCGCCCGGCTACGCAGGCGGCAGGGCTGCGGCACGGGCCGGGCGGCACCATGGCGGCGGCGGCGC
CTGCTGCTGCGGCGGCTTCTTCCGAGGCGCCAGCGGCGAGTGCGACTGCAGAGCCCGAGGCCGGGGACCAGGA
CAGTCGCGAGGTTCGAGTGTTGCAGAGCCTGCGGGGCAAGATCTGTGAAGCAAAAAATTTATTGCCATATCTT
GGACCCCACAAAATGAGAGATTGTTTCTGTACCATAAATTTGGACCAGGAAGAAGTTTATCGTACCCAAGTTG
TGGAAAAATCTTTAAGCCCATTTTTCAGTGAAGAATTTTACTTTGAGATTCCAAGAACTTTCCAGTATTTGTC
TTTCTATGTTTATGATAAGAATGTTTTACAAAGAGATCTCCGTATAGGAAAAGTAGCCATCAAAAAAGAAGAC
TTGTGTAATCACAGTGGCAAAGAAACTTGGTTTTCATTACAGCCTGTTGACTCCAATTCAGAGGTTCAGGGTA
AAGTTCACCTTGAATTAAAACTGAATGAACTGATAACGGAGAATGGAACTGTATGCCAGCAGCTTGTTGTACA
CATCAAGGCATGCCATGGGTTGCCTCTCATAAATGGCCAAAGCTGTGACCCTTATGCAACAGTTTCTCTAGTG
GGCCCTTCTAGGAATGACCAAAAGAAGACAAAAGTAAAGAAGAAAACAAGCAATCCGCAGTTTAATGAAATCT
TTTATTTTGAGGTAACCAGATCCAGTAGTTACACCAGAAAGTCCCAGTTCCAGGTAGAAGAGGAGGACATTGA
AAAGCTAGAAATCAGGATCGACTTGTGGAACAATGGAAACCTAGTCCAAGATGTTTTCCTAGGTGAGATTAAG
GTTCCTGTGAACGTATTAAGAACTGATTCCTCTCATCAAGCCTGGTACTTGCTACAGCCAAGAGACAATGGAA
ACAAGTCATCCAAAACTGATGACCTGGGGTCTCTTCGATTAAATATATGTTATACAGAAGACTACGTGCTTCC
TTCAGAGTACTATGGTCCTTTGAAAACTTTGCTGCTAAAATCACCAGATGTTCAACCAATATCTGCCTCAGCT
GCTTACATTTTGAGTGAAATATGTCGAGATAAAAATGATGCTGTTTTGCCCCTTGTACGACTGCTGCTGCACC
ATGATAAACTTGTTCCTTTTGCCACTGCTGTGGCTGAATTAGACTTGAAGGATACACAAGATGCAAACACAAT
TTTTAGAGGAAATTCCCTGGCTACCCGATGTCTGGATGAGATGATGAAAATAGTGGGAGGGCACTACCTGAAA
GTAACATTAAAACCTATTCTTGATGAGATATGTGACTCCTCAAAATCCTGTGAAATCGATCCTATTAAATTGA
AAGAGGGAGATAATGTAGAAAATAATAAGGAGAATCTGCGCTACTATGTAGACAAGTTATTCAATACAATTGT
AAAATCAAGTATGAGCTGCCCCACTGTAATGTGTGATATCTTTTATTCTCTAAGGCAGATGGCTACTCAGAGA
TTTCCTAATGACCCTCATGTTCAGTATTCTGCAGTGAGCAGCTTTGTATTTCTTCGTTTCTTTGCTGTAGCCG
TAGTATCACCTCATACTTTTCATTTGCGACCTCATCATCCAGATGCACAGACAATTAGAACATTAACTCTCAT
CTCAAAAACTATACAAACTTTGGGAAGCTGGGGGAGTCTGTCCAAAAGCAAGTCAAGTTTCAAAGAGACATTC
ATGTGTGAATTTTTCAAAATGTTTCAAGAAGAAGGATATATTATAGCAGTTAAAAAGTTCTTGGATGAAATTT
CATCTACTGAAACTAAAGAGTCCAGTGGTACGAGTGAGCCTGTGCACCTGAAAGAAGGTGAGATGTATAAAAG
AGCTCAAGGAAGAACTCGGATTGGAAAAAAGAATTTTAAGAAACGATGGTTCTGCTTAACAAGCAGAGAGCTC
ACCTACCACAAACAGCCAGGCAAAGATGCAATCTACACAATCCCAGTAAAAAACATTCTTGCTGTGGAAAAAC
TGGAAGAGAGCTCTTTCAACAAGAAAAATATGTTCCAAGTAATACATACGGAGAAACCACTCTATGTCCAGGC
AAATAACTGTGTAGAAGCTAATGAATGGATAGACGTACTCTGCAGGGTGAGCCGATGCAATCAAAACAGGCTC
AGTTTTTATCATCCCTCTGTGTATCTGAACGGAAATTGGCTCTGCTGTCAGGAGACTGGTGAAAACACTCTCG
GCTGCAAGCCATGTACTGCAGGTGTCCCTGCAGACATCCAAATAGATATTGATGAAGACAGAGAAACAGAAAG
AATTTATTCCCTTTTTACCCTCAGTTTACTTAAGCTGCAGAAGATGGAAGAGGCTTGTGGAACTATTGCAGTC
TATCAAGGACCACAGAAAGAGCCTGATGATTATTCTAACTTTGTAATCGAGGATTCTGTAACAACCTTTAAGA
CAATTCAGCAAATAAAAAGCATAATTGAGAAGCTGGATGAACCTCATGAAAAATATAGGAAGAAAAGATCCAG
TAGTGCAAAATATGGGAGCAAGGAAAATCCAATTGTTGGGAAAGCATCTTAGAGTTTAACAGATTGGTTCAGA
AGAACTGGAAAATATTATTTTTCTTGGAGCTTTTCAATTCATCATGTATTTTGTTCATGGTATTTAAGAATGA
GCATCCGCTTCAATGTCATCTGCCTCCACATTGTATTTAATATTTAATAATTGAAATTAATTGTTTGGGAATC
CTGGTATTGATGTATTACTAGAGAATTTAAAGCCCAAGATTCCCTTCATACCTGTGTGACCAAATCCATGTTT
CTGCAACTTCTTTTTAATCGAAAGAATCTTCCTAGAAAAGCTTTGTAACAAAGGGAGAACTCCTCCGTAGCAA
GAAACCATCTCTTCTTGTAACACTCTGTTCTGTGGACTTGTTTTCACTACCATCAGTGCCTGCTCTATACTGC
CAACATTGTGTTTTACTAGAAAATTCCAATTCATGACAGTTCAGAGCTTTTCATTTAGTTCATGTAGACCCTC
CCACTTTAAAAAAGAAAAAAAATAAGAAGTGTTTTTCCTTTGGTCCATCAGTCATTACAGGTTCCATTCAAGA
CAGTGATAGAAACTTTAGAAAACTGCCAGAGCATACTTGAAAGTTGGTAGATCCTTTTGCCTAAAGATGTAAA
CAAAACTCAAGACAGAAGGAATCAGGGAATATGTGCTATTGTGTGCATCTTGTTTACATTTGGGATCAGTGAT
GGCAAAAGAAGTAATGAGACCACTGAAATTGTTTTCATTGTTTTAAATACCAGGTACTCATTTTCTTGATTTG
AAAGTTTAACATGACTTCTAAGGACATCTCTTCAAAAAGAAAGTAAACAGGGAATGAAGGTGGTGGGAAAATC
ACTTCACTTCACCCACTATTTCATGTTGTAATAAGGGCCACCATAAGGATGCCCTCCTCATAGTGTTCGGTTC
TGATCTTCCTCGAGAACAGTCACTTGGCGCACTTTAATAATCTGGACTGCTCCAGATTTGACTTTAGATACTC
CATGGTATCTAGTCTTTATGATCAGTTGAATGATCAGTGTTTAAGTCTAAAAATAATGCTTTCCTGAAAATGT
TTATATTTCATTGCTGTTTCCTTATTGCCTGCTAGCCAAATGGAATTTGGGCAAGCAACTCTTTGAAGACTTT
TTTACCCTAGAAATTTACTTTTGTCCTAAAAACCCTAACTGTAAATAAGCAGTCGAAGCAAGTTGCCACCTTG
AAGTTGAGGGATACACTTTGCTTCATCAGTATTAAAAACCATGGTACTCTTATTTTGTCTTTTTTAATTCAAA
ACATTTTCTAAATGTGGTACTTTGAACCTTGGAGTATTTACATGTTTCTGTTCAAAACTGTGACTTATTAATG
TAAGTCTGCTAGTAGTACTTTTTTTGTCTGAGCATAAGCTATATTTCAAGATATACTTTGCCAATTTTGTTAT
TGGTGGGTAATCATTTTTAAATTATATTGTTATTAGGTAATTATTTTTAAAGACTACTGGCATACAATCAAAG
TTGTTCCATAAAAGAATATAACTGAGCAATGTATTTCTCTAAATGACTTGTAAAAATCAATGAATTATGTTTA
AGTCTGCATTATGGTAGGTTATTACTCCCAGCTCATTTTAAAGACAAGTTTGAAAGAGTCCTTTAAATTTTAT
AAGCTTGTAGATTCCATAAACTACACTGATTTATACATTTGAAAGAATTTAGCCATTTAATAAATTTCTTATT
CTCAAATTGCATTTAGATTAAAATTCCTTTTAAGAGTGTCTGAAATGTCTGCCTGCGAAGAACATGTAAACAT
TGCCAGATACCAGTCATTATTCTTTAGTGGCTTTTTAGTCCTCTGCTTTTTTTTTTAAGGCAAAAGAAATATG
CATCTAGCATTTCTAATACCAAATCCCCAATTGTGATTGTTTTAAAGATCATGGTATGATCACAGGGACCAAG
GCTGTGTGCATATATAACTCTGGATTTGAAAGGAGGAGAGTTCAGCCATCTAGTTCTTTTCTTCACATGACAC
TTTTAAACAGCCAGTTTGACTGGAATTAACCAACTTGGCACAGCCTAAAAAAGGAGTAATGTGAAATTAAATT
ATTTTTCTTTGAAAGTCTTTTGAAATAAGAGACCATGTAAGTAGGACCCTAAGACAACCAAGGAATTTGCTTC
CAGGGCCCTCATTCCTCTGCTGTTACTTACAAATGTTTTAATTTTTACTACCTGATATTCTTTCTCTACCTTT
TCCACATTTGAAAATATACAAGGCAGCTCACTTCATCCTAAAAACCTAAAAACTCCAGGAAGGAAAGAAAAGG
ATGATATGGGGAAATTTGCCATAAATGCCGGTTCTGGTTCTTAGTTTAGCCATTTCCATCAGTTGAAATTGTG
GTGTGATCTGTTGCTTGTTTCGTTGTTGTTTTGTTTTTGTGTAGCTTTGTTTTTTAATATTCATTTAATAAGA
TGATGGGCAATAGCAAACATTTTTACTCCCAGGAATATAATTTATCAAATTGAACTATTTTATTTGCCAAAGA
AACTTGTTTTTAATATCACGGGTAATGGGTAAGTGTGAAAACTAGGCTTTTTTTTATGAAACAAAAAAAGGTG
ACGAATAATATGGCTTTTGTTTCTGTTTCTGAAGCCTGATTTCATTTATGCCAGGTTTTGAAAAACAGATCTT
TTTCTTAAGTGAAAGCACCTTTAATTTGCTCTAACGGTACATCCTGTAAAGACAGAATTCTGCATAGCCTTTT
AGGTGTTTTTACAGTATGTGAAAAATACAAGACTCATTCTAGAGTTAAATTACCAATCTTGGTTGATATTATC
TGCTTCCATTTTTATTAATTTGGAAATTAATATGTTGCTAGTAAATATTGGTTTTTACAGTACCCAGTTTGAA
AATGTAAGTTATCAAAACTTAATGTAGTGAATTTGTGTAACCATGTTGTATTGTTGAGAATGTATTTTTATTT
AAATTTTATTTTCTTATCAAGAGTATTATAAAAATTAACTTTTGTAACTGTCGCTTGGAAATAACTCAAATAA
ATTACAAGAAGCCTGTC
(SEQ ID NO: 185)
MAAAAPAAAAASSEAPAASATAEPEAGDQDSREVRVLQSLRGKICEAKNLLPYLGPHKMRDCFCTINLDQEEV
YRTQVVEKSLSPFFSEEFYFEIPRTFQYLSFYVYDKNVLQRDLRIGKVAIKKEDLCNHSGKETWFSLQPVDSN
SEVQGKVHLELKLNELITENGTVCQQLVVHIKACHGLPLINGQSCDPYATVSLVGPSRNDQKKTKVKKKTSNP
QFNEIFYFEVTRSSSYTRKSQFQVEEEDIEKLEIRIDLWNNGNLVQDVELGEIKVPVNVLRTDSSHQAWYLLQ
PRDNGNKSSKTDDLGSLRLNICYTEDYVLPSEYYGPLKTLLLKSPDVQPISASAAYILSEICRDKNDAVLPLV
RLLLHHDKLVPFATAVAELDLKDTQDANTIFRGNSLATRCLDEMMKIVGGHYLKVTLKPILDEICDSSKSCEI
DPIKLKEGDNVENNKENLRYYVDKLENTIVKSSMSCPTVMCDIFYSLRQMATQRFPNDPHVQYSAVSSFVELR
FFAVAVVSPHTFHLRPHHPDAQTIRTLTLISKTIQTLGSWGSLSKSKSSFKETFMCEFFKMFQEEGYIIAVKK
FLDEISSTETKESSGTSEPVHLKEGEMYKRAQGRTRIGKKNFKKRWFCLTSRELTYHKQPGKDAIYTIPVKNI
LAVEKLEESSFNKKNMFQVIHTEKPLYVQANNCVEANEWIDVLCRVSRCNQNRLSFYHPSVYLNGNWLCCQET
GENTLGCKPCTAGVPADIQIDIDEDRETERIYSLFTLSLLKLQKMEEACGTIAVYQGPQKEPDDYSNEVIEDS
VTTFKTIQQIKSIIEKLDEPHEKYRKKRSSSAKYGSKENPIVGKAS*

An amino acid sequence for the protein encoded by the RNASE9 gene that is a regulator of T cell exhaustion/dysfunction resistance is available from the UniPROT database at accession no. P60153, cDNA NM_001001673, with gene location at Chr 14: 20556093-20560931.

```
  (SEQ ID NO: 186)
GCTCTACCTCTTGGACTTCCAACTTCTGTGAGTCAACATAGTGCCTCTTGCATTTAAGTGTCTGCAGGAAAAA
TGATGAGAACTCTCATCACCACACACCCACTGCCCCTGCTTCTATTGCCGCAGCAGCTGCTGCAGCTGGTGCA
GTTTCAAGAGGTGGATACAGATTTTGATTTCCCAGAAGAAGATAAAAAAGAAGAATTTGAAGAGTGTTTGGAA
AAATTTTTTAGTACAGGGCCCGCCAGACCACCTACCAAAGAAAAAGTCAAAAGACGTGTCCTTATTGAACCTG
GAATGCCACTAAATCATATAGAGTACTGTAACCATGAAATCATGGGAAAAAATGTTTACTACAAACACCGTTG
GGTGGCAGAACATTACTTCCTTCTTATGCAATATGACGAGCTCCAAAAAATCTGTTACAACAGATTTGTGCCA
TGTAAGAATGGAATTAGGAAATGTAACAGGAGCAAAGGTCTTGTAGAAGGAGTGTATTGTAATTTAACAGAAG
CATTTGAAATACCAGCGTGTAAATACGAATCACTTTATAGGAAGGGCTACGTCCTTATCACTTGTTCATGGCA
AAATGAAATGCAAAAACGTATTCCTCATACTATAAATGATCTCGTGGAGCCACCTGAACACAGAAGTTTCCTC
AGTGAGGATGGTGTCTTTGTCATATCGCCCTAGCAGAGCGTTCTCTGAGAGCTTAGGGTGGGAGGAATATTTC
TCTCCATACTGATCCTTATAGATCAAAGTTGAGAATGGTAATTTTGCTTCCATTCACTTTAATCACCCACCTT
CCTTTCTTCCTTTCCCAACACCTCACAAAAGCACCTTAGGCTAGGTTCACAGACGGTGCTCTGAGATGTGGTC
TCATGCAGGTCCCCTAAGTTAGCATGCCTTGAATGTGTTACGGATACAGATGCCTGCAGTGGAAGGATGACCA
GCACCAAGAGGAAAAGGAAATGGTTGCCTGAAGTGTGCTCCAAAGACACATCCATGTGATTCTCTAGTTCAGC
AGAAAGAATAAAAATTGCTTCAATCTA
  (SEQ ID NO: 187)
MMRTLITTHPLPLLLLPQQLLQLVQFQEVDTDFDFPEEDKKEEFEECLEKFFSTGPARPPTKEKVKRRVLIEP
GMPLNHIEYCNHEIMGKNVYYKHRWVAEHYFLLMQYDELQKICYNRFVPCKNGIRKCNRSKGLVEGVYCNLTE
AFETPACKYESLYRKGYVLITCSWQNEMQKRIPHTINDLVEPPEHRSFLSEDGVFVISP*
```

An amino acid sequence for the protein encoded by the RNF7 gene that is a regulator of T cell exhaustion/dysfunction resistance is available from the UniPROT database at accession no. Q9UBF6, cDNA NM_001201370, with gene location at Chr 3: 141738249-141747560.

(SEQ ID NO: 188)
AGAGCCGCCCTAGCCTTCCGCCTTCCCCAAGCCAACGTCTCCGCCGTCGG
CTCCGCGGCGCCGCCATGGCCGACGTGGAAGACGGAGAGGAAACCTGCGC
CCTGGCCTCTCACTCCGGGAGCTCAGGCTCCAAGTCGGGAGGCGACAAGA
TGTTCTCCCTCAAGAAGTGGAACGCGGTGGCCATGTGGAGCTGGGACGTG
GAGTGCGATACGTGCGCCATCTGCAGGGTCCAGGTGATGGTGGTCTGGGG
AGAATGTAATCATTCCTTCCACAACTGCTGCATGTCCCTGTGGGTGAAAC
AGAACAATCGCTGCCCTCTCTGCCAGCAGGACTGGGTGGTCCAAAGAATC
GGCAAATGAGAGTGGTTAGAAGGCTTCTTAGCGCAGTTGTTCAGAGCCCT
GGTGGATCTTGTAATCCAGTGCCCTACAAAGGCTAGAACACTACAGGGGA
TGAATTCTTCAAATAGGAGCCGATGGATCTGTGGTCCTTTGGGACTCATC
AAAGCCTTGGTTTAGCATTTTGTCAGTTTTATCTTCAGAAATTCTCTGCG
ATTAAGAAGATAATTTATTAAAGGTGGTCCTTCCTACCTCTGTGGTGTGT
GTCGCGCACACAGCTTAGAAGTGCTATAAAAAAGGAAAGAGCTCCAAATT
GAATCACCTTTATAATTTACCCATTTCTATACAACAGGCAGTGGAAGCAG
TTTCAGAGAACTTTTTGCATGCTTATGGTTGATCAGTTAAAAAAGAATGT
TACAGTAACAAATAAAGTGCAGTTTAAAACCC
(SEQ ID NO: 189)
MADVEDGEETCALASHSGSSGSKSGGDKMFSLKKWNAVAMWSWDVECDTC
AICRVQVMVVWGECNHSFHNCCMSLWVKQNNRCPLCQQDWVVQRIGK*

An amino acid sequence for the protein encoded by the RPS6KA1 gene that is a regulator of T cell exhaustion/dysfunction resistance is available from the UniPROT database at accession no. Q15418, cDNA NM_001006665, with gene location at Chr 1: 26529761-26575030.

```
  (SEQ ID NO: 190)
CCCCGGACTCTGCCTGCTCCTGCCATGGTGCCGTGGCCCTGCTGGGCGGATGGAGCAGGATCCCAAGCCGCCC
CGTCTGCGGCTCTGGGCCCTGATCCCCTGGCTTCCCAGGAAGCAGCGGCCCAGGATCAGCCAGACCTCTCTGC
CTGTCCCTGGCCCTGGCTCTGGCCCCCAGCGGGACTCGGATGAGGGCGTCCTCAAGGAGATCTCCATCACGCA
CCACGTCAAGGCTGGCTCTGAGAAGGCTGATCCATCCCATTTCGAGCTCCTCAAGGTTCTGGGCCAGGGATCC
TTTGGCAAAGTCTTCCTGGTGCGGAAAGTCACCCGGCCTGACAGTGGGCACCTGTATGCTATGAAGGTGCTGA
AGAAGGCAACGCTGAAAGTACGTGACCGCGTCCGGACCAAGATGGAGAGAGACATCCTGGCTGATGTAAATCA
CCCATTCGTGGTGAAGCTGCACTATGCCTTCCAGACCGAGGGCAAGCTCTATCTCATTCTGGACTTCCTGCGT
GGTGGGGACCTCTTCACCCGGCTCTCAAAAGAGGTGATGTTCACGGAGGAGGATGTGAAGTTTTACCTGGCTG
AGCTGGCTCTGGGCCTGGATCACCTGCACAGCCTGGGTATCATTTACAGAGACCTCAAGCCTGAGAACATCCT
TCTGGATGAGGAGGGCCACATCAAACTCACTGACTTTGGCCTGAGCAAAGAGGCCATTGACCACGAGAAGAAG
GCCTATTCTTTCTGCGGGACAGTGGAGTACATGGCCCCTGAGGTCGTCAACCGCCAGGGCCACTCCCATAGTG
CGGACTGGTGGTCCTATGGGGTGTTGATGTTTGAGATGCTGACGGGCTCCCTGCCCTTCCAGGGGAAGGACCG
GAAGGAGACCATGACACTGATTCTGAAGGCGAAGCTAGGCATGCCCCAGTTTCTGAGCACTGAAGCCCAGAGC
CTCTTGCGGGCCCTGTTCAAGCGGAATCCTGCCAACCGGCTCGGCTCCGGCCCTGATGGGGCAGAGGAAATCA
AGCGGCATGTCTTCTACTCCACCATTGACTGGAATAAGCTATACCGTCGTGAGATCAAGCCACCCTTCAAGCC
AGCAGTGGCTCAGCCTGATGACACCTTCTACTTTGACACCGAGTTCACGTCCCGCACACCCAAGGATTCCCCA
GGCATCCCCCCCAGCGCTGGGGCCCATCAGCTGTTCCGGGGCTTCAGCTTCGTGGCCACCGGCCTGATGGAAG
ACGACGGCAAGCCTCGTGCCCCGCAGGCACCCCTGCACTCGGTGGTACAGCAACTCCATGGGAAGAACCTGGT
TTTTAGTGACGGCTACGTGGTAAAGGAGACAATTGGTGTGGGCTCCTACTCTGAGTGCAAGCGCTGTGTCCAC
AAGGCCACCAACATGGAGTATGCTGTCAAGGTCATTGATAAGAGCAAGCGGGATCCTTCAGAAGAGATTGAGA
TTCTTCTGCGGTATGGCCAGCACCCCAACATCATCACTCTGAAAGATGTGTATGATGATGGCAAACACGTGTA
CCTGGTGACAGAGCTGATGCGGGGTGGGGAGCTGCTGGACAAGATCCTGCGGCAGAAGTTCTTCTCAGAGCGG
GAGGCCAGCTTTGTCCTGCACACCATTGGCAAAACTGTGGAGTATCTGCACTCACAGGGGGTTGTGCACAGGG
ACCTGAAGCCCAGCAACATCCTGTATGTGGACGAGTCCGGGAATCCCGAGTGCCTGCGCATCTGTGACTTTGG
TTTTGCCAAACAGCTGCGGGCTGAGAATGGGCTCCTCATGACACCTTGCTACACAGCCAACTTTGTGGCGCCT
GAGGTGCTGAAGCGCCAGGGCTACGATGAAGGCTGCGACATCTGGAGCCTGGGCATTCTGCTGTACACCATGC
TGGCAGGATATACTCCATTTGCCAACGGTCCCAGTGACACACCAGAGGAAATCCTAACCCGGATCGGCAGTGG
GAAGTTTACCCTCAGTGGGGGAAATTGGAACACAGTTTCAGAGACAGCCAAGGACCTGGTGTCCAAGATGCTA
CACGTGGATCCCCACCAGCGCCTCACAGCTAAGCAGGTTCTGCAGCATCCATGGGTCACCCAGAAAGACAAGC
TTCCCCAAAGCCAGCTGTCCCACCAGGACCTACAGCTTGTGAAGGGAGCCATGGCTGCCACGTACTCCGCACT
CAACAGCTCCAAGCCCACCCCCCAGCTGAAGCCCATCGAGTCATCCATCCTGGCCCAGCGGCGAGTGAGGAAG
TTGCCATCCACCACCCTGTGAGGCACCAGGGCATTCGGGCCACAGGGCGGTGCTAGCTTGACACAGTCAGCAT
GCTTCCCAGAGGGAGCAGGCCGG
  (SEQ ID NO: 191)
MEQDPKPPRLRLWALIPWLPRKQRPRISQTSLPVPGPGSGPQRDSDEGVLKEISITHHVKAGSEKADPSHFEL
LKVLGQGSFGKVFLVRKVTRPDSGHLYAMKVLKKATLKVRDRVRTKMERDILADVNHPFVVKLHYAFQTEGKL
YLILDFLRGGDLFTRLSKEVMFTEEDVKFYLAELALGLDHLHSLGIIYRDLKPENILLDEEGHIKLTDFGLSK
EAIDHEKKAYSFCGTVEYMAPEVVNRQGHSHSADWWSYGVLMFEMLTGSLPFQGKDRKETMTLILKAKLGMPQ
FLSTEAQSLLRALFKRNPANRLGSGPDGAEEIKRHVFYSTIDWNKLYRREIKPPFKPAVAQPDDTFYFDTEFT
SRTPKDSPGIPPSAGAHQLFRGFSFVATGLMEDDGKPRAPQAPLHSVVQQLHGKNLVFSDGYVVKETIGVGSY
SECKRCVHKATNMEYAVKVIDKSKRDPSEEIEILLRYGQHPNIITLKDVYDDGKHVYLVTELMRGGELLDKIL
RQKFFSEREASFVLHTIGKTVEYLHSQGVVHRDLKPSNILYVDESGNPECLRICDFGFAKQLRAENGLLMTPC
YTANFVAPEVLKRQGYDEGCDIWSLGILLYTMLAGYTPFANGPSDTPEEILTRIGSGKFTLSGGNWNTVSETA
KDLVSKMLHVDPHQRLTAKQVLQHPWVTQKDKLPQSQLSHQDLQLVKGAMAATYSALNSSKPTPQLKPIESSI
LAQRRVRKLPSTTL*
```

An amino acid sequence for the protein encoded by the SBNO2 gene that is a regulator of T cell exhaustion/dysfunction resistance is available from the UniPROT database at accession no. Q9Y2G9, cDNA NM_001100122, with gene location at Chr 19: 1107637-1174268.

(SEQ ID NO: 192)
AATGCTGGGGCTGCTGCCGCGGTCATGAGGGAGCCGCTCCCGGGCAGCGCTTCCTGGGGGACCCCTGGCCCCC
CGAGCGCCGGGACCATGTCCCAGCTGCAGCTGTGGCTGCAGTTTGAGGCTCTGAACAAGGACTCCTCCTATTT
TGAGGACTTCTCCAACATCTCCATCTTCTCCTCGTCCGTGGACTCCCTGTCGGACATCGTGGACACGCCCGAC
TTCCTGCCGGCTGACAGCCTCAACCAGGTGTCCACCATCTGGGACGATAACCCTGCCCCCTCCACCCACGATA
AGCTGTTCCAGCTCAGCAGGCCGTTTGCAGGCTTCGAGGACTTTCTGCCCTCCCACAGCACCCCGCTTCTCGT
CAGCTACCAGGAGCAGAGTGTGCAGAGCCAGCCAGAGGAGGAGGACGAGGCTGAGGAGGAGGAGGCGGAGGAG
CTGGGGCACACAGAGACCTACGCCGACTACGTGCCGTCCAAGTCCAAGATCGGGAAGCAGCACCCAGACCGCG
TGGTGGAGACCAGCACACTGTCCAGCGTCCCACCCCCAGACATCACCTACACCCTGGCCCTGCCCTCGGACAG
CGGGGCCCTGTCTGCCCTGCAGCTAGAGGCCATCACCTACGCCTGCCAGCAACACGAGGTCCTGCTCCCCAGC
GGGCAGCGCGCGGGCTTTCTCATCGGCGATGGGGCCGGCGTGGGCAAAGGCCGGACGGTGGCCGGAGTCATCC
TGGAGAACCACCTGCGCGGCCGGAAGAAAGCATTGTGGTTCAGCGTCTCCAACGACCTCAAGTACGATGCGGA
GCGCGACCTGCGGGACATCGAAGCCACGGGCATCGCGGTGCACGCGCTCAGCAAGATCAAGTACGGTGACACC
ACTACCTCAGAGGGCGTCCTCTTCGCCACCTACTCCGCCCTGATTGGGGAGAGCCAGGCCGGCGGCCAGCACC
GCACTCGCCTCCGGCAGATCCTGGACTGGTGTGGGGAGGCCTTCGAGGGCGTCATCGTGTTCGACGAGTGTCA
CAAAGCCAAGAATGCCGGCTCCACCAAGATGGGCAAGGCTGTGCTAGACCTGCAGAACAAGCTGCCCCTGGCC
CGCGTGGTCTACGCCAGCGCCACAGGTGCCTCTGAGCCTCGGAACATGATCTACATGAGCCGCTTGGGTATCT
GGGGCGAGGGCACACCCTTCCGGAACTTTGAGGAGTTCCTGCACGCCATCGAGAAGAGGGGCGTTGGCGCCAT
GGAGATCGTGGCCATGGACATGAAGGTCAGCGGCATGTACATCGCACGCCAGCTCAGCTTCTCCGGCGTCACC
TTCCGCATCGAGGAGATCCCGCTGGCCCCAGCCTTCGAGTGCGTCTACAACCGCGCGGCCCTGCTGTGGGCCG
AGGCCCTGAACGTGTTCCAGCAGGCGGCCGACTGGATCGGCCTGGAGTCGCGCAAGTCCCTGTGGGGCCAGTT
CTGGTCGGCACACCAGCGCTTCTTCAAGTATCTGTGCATCGCAGCCAAGGTGCGCCGGCTGGTGGAGCTGGCC
CGAGAGGAGCTGGCGCGAGACAAGTGCGTGGTCATCGGGCTGCAGTCCACGGGCGAGGCGCGCACGCGGGAGG
TGCTGGGGGAGAACGATGGGCACCTCAACTGCTTCGTCTCGGCCGCTGAAGGCGTGTTCCTGTCGCTAATTCA
GAAGCACTTTCCGTCCACCAAGAGAAAGCGGGACAGAGGAGCGGGCAGCAAGCGGAAACGGCGACCTCGGGGA
CGCGGGGCCAAAGCCCCCCGGCTGGCGTGCGAGACAGCGGGCGTCATCCGCATCAGTGACGACAGCAGCACGG
AGTCGGACCCTGGCCTGGACAGCGACTTCAACTCCTCCCCCGAGTCCCTGGTGGATGACGACGTTGTCATCGT
TGATGCAGTCGGGCTCCCCAGTGACGACCGGGGACCCCTGTGCCTCCTGCAGAGAGACCCGCATGGCCCCGGG
GTCCTGGAGCGGGTGGAGCGGCTGAAGCAGGATCTGCTGGACAAAGTGCGGCGGCTGGGCCGGGAACTGCCAG
TCAACACCCTGGACGAGCTCATCGACCAGCTGGGCGGCCCCCAGCGGGTGGCGGAGATGACCGGCAGGAAAGG
CCGCGTGGTGTCCAGGCCCGACGGGACGGTGGCCTTCGAGTCGCGGGCAGAGCAGGGTCTGTCCATCGACCAC
GTGAACCTCAGGGAGAAGCAGCGCTTCATGAGCGGCGAGAAGCTCGTGGCCATCATCTCGGAGGCCTCCAGCT
CGGGTGTCTCCCTCCAAGCCGACCGCCGTGTCCAGAACCAGCGGCGCCGCGTGCACATGACCTTGGAGCTGCC
GTGGAGCGCCGACCGCGCCATCCAGCAGTTCGGCCGCACCCACCGGTCCAACCAGGTCTCCGCGCCAGAGTAT
GTCTTCCTCATCTCGGAGCTGGCCGGGGAGCGCCGGTTCGCCTCCATCGTGGCCAAGCGCCTGGAGAGTCTGG
GGGCCCTGACCCACGGAGACCGCCGCGCCACGGAGTCCCGTGACCTCAGCAAGTACAACTTTGAGAACAAGTA
TGGCACCCGGGCCCTGCACTGTGTCCTCACCACCATCCTGAGCCAGACTGAGAACAAAGTGCCTGTGCCCCAG
GGATACCCTGGAGGGGTCCCCACCTTCTTCCGGGACATGAAGCAGGGCCTGCTGTCTGTGGGCATTGGTGGCC
GGGAGTCCCGGAATGGCTGCCTGGACGTGGAGAAGGACTGTTCCATCACCAAGTTCCTGAACCGCATCCTGGG
GCTGGAGGTGCACAAGCAGAACGCCCTGTTCCAGTACTTCTCAGACACCTTCGACCACCTCATCGAGATGGAC
AAGCGGGAGGGCAAATACGACATGGGCATCCTGGACCTTGCTCCCGGTATCGAGGAGATCTACGAGGAGAGCC
AGCAGGTGTTCCTGGCTCCCGGGCACCCGCAGGACGGGCAGGTGGTCTTCTACAAGATCAGCGTGGACCGCGG
CCTGAAGTGGGAGGACGCCTTTGCCAAGTCGCTGGCGCTGACGGGCCCCTATGACGGCTTCTACCTCTCCTAC
AAGGTCCGCGGTAACAAGCCCAGCTGCCTGCTGGCGGAGCAGAACCGCGGCCAGTTCTTCACGGTGTACAAGC
CCAACATCGGCCGGCAGAGCCAGCTGGAGGCCCTGGACAGCCTCCGCCGCAAGTTCCACCGGGTCACCGCGGA
GGAGGCCAAGGAGCCCTGGGAGAGTGGCTACGCTTTGTCGCTGACGCACTGCAGCCACAGCGCCTGGAACCGG
CACTGCCGGCTGGCGCAGGAGGGTAAGGACTGCCTGCAGGGGCTGCGGCTGCGGCACCACTACATGCTGTGCG
GCGCGCTGCTGCGCGTGTGGGGCCGCATCGCCGCCGTCATGGCCGACGTCAGCAGCAGCAGCTACCTGCAGAT
CGTGCGGCTGAAGACCAAGGACAGGAAGAAGCAAGTGGGCATCAAGATCCCCGAGGGCTGCGTGCGCCGGGTG
CTGCAGGAGCTGCGGCTGATGGATGCGGACGTGAAGCGCAGGCAGGCGCCCGCCCTGGGCTGCCCCGCCCCGC
CCGCCCCGCGCCCGCTGGCGCTGCCTTGCGGCCCCGGAGAGGTGCTGGACCTCACCTACAGCCCCCCGGCCGA
GGCCTTCCCGCCGCCCCCGCACTTCTCTTTCCCGGCGCCGCTGTCCCTGGACGCCGGCCCCGGCGTCGTGCCG
CTGGGCACCCCCGACGCCCAGGCCGACCCTGCGGCCCTCGCGCACCAGGGCTGCGACATCAACTTCAAGGAGG
TGCTGGAGGACATGCTGCGCTCGCTGCACGCGGGGCCGCCCTCCGAGGGCGCGCTGGGGGAGGGCGCGGGGGC
GGGGGGCGCGGCGGGCGGTGGTCCCGAGCGGCAGAGCGTGATCCAGTTCAGCCCACCCTTCCCCGGCGCCCAG
GCTCCTCTCTGACACGCCTTTAGGCGAAACATGCCCCAAGACACAGGGACCGTTTCTCCCCTAGGAGCAGCGG
TGGGGAGCAGGGCCAAGGTCCCCTGACCACTGCTCAGAGGAGCCCTAGGCCCTGGCCGCAGTGCCTTCAGCGC
CCGACCCGGGCCCCCACCTGGTCAGCCCTGGCGGGGCCCACTCAGGACAGCTGGGGGCCGGGGCGTGGCAGGG
CCCTCTCTGTGCCTCTCCTCCCAAGTAGGAAGGGGCTCCGGGTGGCTGCTCTGGGACTGGGCACCCACAAGGG
CTCAGTGGGCCCAAACCCTTGAAATCCGTGAAACCGGGTGGTCCCAAGAGCTAGAAACTCAGGAAACCCCAGG
TGCTCAGGGCCCCGCGTCTCGGGGGCTCCGTGGGGCAGACCCCTGCTAATATATGCAATTCTCCCTCCCCCAG
CCCTTCCCTGACCCCTAAGTTATTGCCCGCTCACCTCTCCCAGGCCCCAGGCCGCGGAGCTGGCAGGGTGGCG
CCTGCGGTTTCTATGTATTTATAGCAAGTTCTGATGTACATATGTAAAGGACTTTTTTAAATATATGTGCCTT
TTGCCTACTTC
(SEQ ID NO: 193)
MREPLPGSASWGTPGPPSAGTMSQLQLWLQFEALNKDSSYFEDFSNISIFSSSVDSLSDIVDTPDFLPADSLN
QVSTIWDDNPAPSTHDKLFQLSRPFAGFEDFLPSHSTPLLVSYQEQSVQSQPEEEDEAEEEEAEELGHTETYA
DYVPSKSKIGKQHPDRVVETSTLSSVPPPDITYTLALPSDSGALSALQLEAITYACQQHEVLLPSGQRAGFLI
GDGAGVGKGRTVAGVILENHLRGRKKALWFSVSNDLKYDAERDLRDIEATGIAVHALSKIKYGDTTTSEGVLF
ATYSALIGESQAGGQHRTRLRQILDWCGEAFEGVIVEDECHKAKNAGSTKMGKAVLDLQNKLPLARVVYASAT
GASEPRNMIYMSRLGIWGEGTPFRNFEEFLHAIEKRGVGAMEIVAMDMKVSGMYIARQLSESGVTFRIEEIPL
APAFECVYNRAALLWAEALNVFQQAADWIGLESRKSLWGQFWSAHQRFFKYLCIAAKVRRLVELAREELARDK
CVVIGLQSTGEARTREVLGENDGHLNCFVSAAEGVFLSLIQKHFPSTKRKRDRGAGSKRKRRPRGRGAKAPRL
ACETAGVIRISDDSSTESDPGLDSDENSSPESLVDDDVVIVDAVGLPSDDRGPLCLLQRDPHGPGVLERVERL
KQDLLDKVRRLGRELPVNTLDELIDQLGGPQRVAEMTGRKGRVVSRPDGTVAFESRAEQGLSIDHVNLREKQR
FMSGEKLVAIISEASSSGVSLQADRRVQNQRRRVHMTLELPWSADRAIQQFGRTHRSNQVSAPEYVFLISELA
GERRFASIVAKRLESLGALTHGDRRATESRDLSKYNFENKYGTRALHCVLTTILSQTENKVPVPQGYPGGVPT
FFRDMKQGLLSVGIGGRESRNGCLDVEKDCSITKFLNRILGLEVHKQNALFQYFSDTFDHLIEMDKREGKYDM
GILDLAPGIEEIYEESQQVFLAPGHPQDGQVVFYKISVDRGLKWEDAFAKSLALTGPYDGFYLSYKVRGNKPS
CLLAEQNRGQFFTVYKPNIGRQSQLEALDSLRRKFHRVTAEEAKEPWESGYALSLTHCSHSAWNRHCRLAQEG
KDCLQGLRLRHHYMLCGALLRVWGRIAAVMADVSSSSYLQIVRLKTKDRKKQVGIKIPEGCVRRVLQELRLMD
ADVKRRQAPALGCPAPPAPRPLALPCGPGEVLDLTYSPPAEAFPPPPHFSFPAPLSLDAGPGVVPLGTPDAQA
DPAALAHQGCDINFKEVLEDMLRSLHAGPPSEGALGEGAGAGGAAGGGPERQSVIQFSPPFPGAQAPL*

An amino acid sequence for the protein encoded by the SND1 gene that is a regulator of T cell exhaustion/dysfunction resistance is available from the UniPROT database at accession no. Q7KZF4, cDNA NM_014390, with gene location at Chr 7: 127652194-128092609.

```
  (SEQ ID NO: 194)
CTCTTTCGCTCCGTGTCCCGCTGCTGCTCCTGTGAGCGCCCGGCGAGTCCGTCCCGTCCACCGTCCGCAGCTG
GTAGCCAGCCTGCCCCTCGCCTCGACTCCCTTTCACCAACACCGACACCCACATTGACACCTCCAGTCCGGCC
AGCCGCTCCACTCGTTGCCTTTGCATCTCCACACATGGCGTCCTCCGCGCAGAGCGGCGGCTCCTCCGGGGGA
CCCGCGGTCCCCACCGTGCAGCGGGGCATCATCAAGATGGTCCTCTCAGGGTGCGCCATCATTGTCCGAGGTC
AGCCTCGTGGTGGGCCTCCTCCTGAGCGGCAGATCAACCTCAGCAACATTCGTGCTGGAAATCTTGCTCGCCG
GGCAGCCGCCACACAACCTGATGCAAAGGATACCCCTGATGAGCCCTGGGCATTTCCAGCTCGAGAGTTCCTT
CGAAAGAAGCTGATTGGGAAGGAAGTCTGTTTCACGATAGAAAACAAGACTCCCCAGGGGCGAGAGTATGGCA
TGATCTACCTTGGAAAAGATACCAATGGGGAAAACATTGCAGAATCACTGGTTGCAGAGGGCTTAGCCACCCG
GAGAGAAGGCATGAGAGCTAATAATCCTGAGCAGAACCGGCTTTCAGAATGTGAAGAACAAGCAAAGGCAGCC
AAGAAAGGGATGTGGAGTGAGGGGAACGGTTCACATACTATCCGGGATCTCAAGTATACCATTGAAAACCCAA
GGCACTTTGTGGACTCACACCACCAGAAGCCTGTTAATGCTATCATCGAGCATGTGCGGGACGGCAGTGTGGT
CAGGGCCCTGCTCCTCCCAGATTACTACCTGGTTACAGTCATGCTGTCAGGCATCAAGTGCCCAACTTTTCGA
CGGGAAGCAGATGGCAGTGAAACTCCAGAGCCTTTTGCTGCAGAAGCCAAATTTTTCACTGAGTCGCGACTGC
TTCAGAGAGATGTTCAGATCATTCTGGAGAGCTGCCACAACCAGAACATTCTGGGTACCATCCTTCATCCAAA
TGGCAACATCACAGAGCTCCTCCTGAAGGAAGGTTTCGCACGCTGTGTGGACTGGTCGATTGCAGTTTACACC
CGGGGCGCAGAAAAGCTGAGGGCGGCAGAGAGGTTTGCCAAAGAGCGCAGGCTGAGAATATGGAGAGACTATG
TGGCTCCCACAGCTAATTTGGACCAAAAGGACAAGCAGTTTGTTGCCAAGGTGATGCAGGTTCTGAATGCTGA
TGCCATTGTTGTGAAGCTGAACTCAGGCGATTACAAGACGATTCACCTGTCCAGCATCCGACCACCGAGGCTG
GAGGGGGAGAACACCCAGGATAAGAACAAGAAACTGCGTCCCCTGTATGACATTCCTTACATGTTTGAGGCCC
GGGAATTTCTTCGAAAAAAGCTTATTGGGAAGAAGGTCAATGTGACGGTGGACTACATTAGACCAGCCAGCCC
AGCCACAGAGACAGTGCCTGCCTTTTCAGAGCGTACCTGTGCCACTGTCACCATTGGAGGAATAAACATTGCT
GAGGCTCTTGTCAGCAAAGGTCTAGCCACAGTGATCAGATACCGGCAGGATGATGACCAGAGATCATCACACT
ACGATGAACTGCTTGCTGCAGAGGCCAGAGCTATTAAGAATGGCAAAGGATTGCATAGCAAGAAGGAAGTGCC
TATCCACCGTGTTGCAGATATATCTGGGGATACCCAAAAAGCAAAGCAGTTCCTGCCTTTTCTTCAGCGGGCA
GGTCGTTCTGAAGCTGTGGTGGAATACGTCTTCAGTGGTTCTCGTCTCAAACTCTATTTGCCAAAGGAAACTT
GCCTTATCACCTTCTTGCTTGCAGGCATTGAATGCCCCAGAGGAGCCCGAAACCTCCCAGGCTTGGTGCAGGA
AGGAGAGCCCTTCAGCGAGGAAGCTACACTTTTCACCAAGGAACTGGTGCTGCAGCGAGAGGTGGAGGTGGAG
GTGGAGAGCATGGACAAGGCCGGCAACTTTATCGGCTGGCTGCACATCGACGGTGCCAACCTGTCCGTCCTGC
TGGTGGAGCACGCGCTCTCCAAGGTCCACTTCACCGCCGAACGCAGCTCCTACTACAAGTCCCTGCTGTCTGC
CGAGGAGGCCGCAAAGCAGAAGAAAGAGAAGGTCTGGGCCCACTATGAGGAGCAGCCCGTGGAGGAGGTGATG
CCAGTGCTGGAGGAGAAGGAGCGATCTGCTAGCTACAAGCCCGTGTTTGTGACTGAGATCACTGATGACCTGC
ACTTCTACGTGCAGGATGTGGAGACCGGCACCCAGTTGGAGAAGCTGATGGAGAACATGCGCAATGACATTGC
CAGTCACCCCCCTGTAGAGGGCTCCTATGCCCCCCGCAGGGGAGAGTTCTGCATTGCCAAATTTGTAGATGGA
GAATGGTACCGTGCCCGAGTAGAGAAAGTCGAGTCTCCTGCCAAAATACATGTCTTCTACATTGACTACGGCA
ACAGAGAGGTCCTGCCATCCACCCGCCTGGGTACCCTATCACCTGCCTTCAGCACTCGGGTGCTGCCAGCTCA
AGCCACGGAGTATGCCTTCGCCTTCATCCAGGTGCCCCAAGATGATGATGCCCGCACGGACGCCGTGGACAGC
GTAGTTCGGGATATCCAGAACACTCAGTGCCTGCTCAACGTGGAACACCTGAGTGCCGGCTGCCCCCATGTCA
CCCTGCAGTTTGCAGATTCCAAGGGCGATGTGGGGCTGGGCTTGGTGAAGGAAGGGCTGGTCATGGTGGAGGT
GCGCAAGGAGAAACAGTTCCAGAAAGTGATCACAGAATACCTGAATGCCCAAGAGTCAGCCAAGAGCGCCAGG
CTGAACCTGTGGCGCTATGGAGACTTTCGAGCTGATGATGCAGACGAATTTGGCTACAGCCGCTAAGGAGGGG
ATCGGGTTTGGCCCCCAGCCCCGCTCACGCCAGTCCCTCTTCCTCTGCCGGGAGGGTGTTTTCAACTCCAAAC
CCCAGAGAGGGGTTGTAGATTGGGTCCAGCTTTGCTTCAGTGTGTGGAAATGTCTCGTGGGGTGGCATCGGGG
CTGCGGGGTGGGGACCCCAAGGCTTTCTGGGGCAGACCCTTGTCCTCTGGGATGATGGGCACTGCTATCCACA
GTCTCTGCCAGTTGGTTTTATTTGGAGGTTTGTGGGCTTTTTTTAAAAAAAAAAAGTCCTCAAATCAGGAAGA
AACATCAAAGACTATGTCCTAGTGGAGGGAGTAATCCTAACACCCAGGCTGGCCGCCAGCTGGCACCTGCCTC
TATCCCAGACTGCCCTCGTCCCAGCTCTCTGTCCAACTGTTGATTATGTGATTTTTCTGATACGTCCATTCTC
AAATGCCAGTGTGTTCACATCTTCGCTCTGGCCAGCCCATTCTGTATTTAAAGCTTTTGAGGCCCAATAAAAT
AGTACGTGCTGTCTGCA
  (SEQ ID NO: 195)
MASSAQSGGSSGGPAVPTVQRGIIKMVLSGCAIIVRGQPRGGPPPERQINLSNIRAGNLARRAAATQPDAKDT
PDEPWAFPAREFLRKKLIGKEVCFTIENKTPQGREYGMIYLGKDTNGENIAESLVAEGLATRREGMRANNPEQ
NRLSECEEQAKAAKKGMWSEGNGSHTIRDLKYTIENPRHFVDSHHQKPVNAIIEHVRDGSVVRALLLPDYYLV
TVMLSGIKCPTFRREADGSETPEPFAAEAKFFTESRLLQRDVQIILESCHNQNILGTILHPNGNITELLLKEG
FARCVDWSIAVYTRGAEKLRAAERFAKERRLRIWRDYVAPTANLDQKDKQFVAKVMQVLNADAIVVKLNSGDY
KTIHLSSIRPPRLEGENTQDKNKKLRPLYDIPYMFEAREFLRKKLIGKKVNVTVDYIRPASPATETVPAFSER
TCATVTIGGINIAEALVSKGLATVIRYRQDDDQRSSHYDELLAAEARAIKNGKGLHSKKEVPIHRVADISGDT
QKAKQFLPFLQRAGRSEAVVEYVESGSRLKLYLPKETCLITFLLAGIECPRGARNLPGLVQEGEPFSEEATLE
TKELVLQREVEVEVESMDKAGNFIGWLHIDGANLSVLLVEHALSKVHFTAERSSYYKSLLSAEEAAKQKKEKV
WAHYEEQPVEEVMPVLEEKERSASYKPVFVTEITDDLHFYVQDVETGTQLEKLMENMRNDIASHPPVEGSYAP
RRGEFCIAKFVDGEWYRARVEKVESPAKIHVFYIDYGNREVLPSTRLGTLSPAFSTRVLPAQATEYAFAFIQV
PQDDDARTDAVDSVVRDIQNTQCLLNVEHLSAGCPHVTLQFADSKGDVGLGLVKEGLVMVEVRKEKQFQKVIT
EYLNAQESAKSARLNLWRYGDFRADDADEFGYSR*
```

An amino acid sequence for the protein encoded by the ST8SIA5 gene that is a regulator 55 of T cell exhaustion/dysfunction resistance is available from the UniPROT database at accession no. 015466. cDNA NM_001307986, with gene location at Chr 18: 46667821-46759257.

```
  (SEQ ID NO: 196)
AAATCGGCGGCTACCCGGCACCGGCGCCTGGAGGTAAAGGTACCCGGGGGTGGGGAAGGCCCCTGGCCGCGGC
AGAGCTCCCTGCCACCGGCCCTGTCCTGCTTGCCGCCCCAGCGCCTGCGACGCTTGGATGCCCCCAGCTCCGG
CCGCGCCAACCAGAAACTTTGCCCCTCGAAGTCCCCCGCCTGCCTGACCGCACCGTGAGGCGCCAAGTCGGCC
GCGCGCCCCCCGGGGCCCCGGAACCCCGCGAGTCATTGCCTGGCCGCCCCGTACCCCGCGCCCGCGGCGCCCG
GTAGCCAGGATGCGCTACGCGGACCCCTCGGCCAACCGGGATTTGTTGGGGAGCCGAACTTTGCTCTTCATCT
TCATCTGCGCCTTTGCCTTGGTGACCTTGCTGCAACAGATCCTGTATGGCAGGAACTACATTAAGAGAGGCCT
CCAGTTTGGTTGGCAGAGTGGTGACCAGCTGACCAATTGGACGGGGCTCTTTAATGTCACGGATGATCCAGCA
GTGCAGAGCGGCAGTGACTCCAGCTCCAGGTACTTTGAATTTTATGAGGGGCCTTTTGAATATAACTCCACAA
GATGCCTGGAGCTGAGGCACGAAATATTGGAAGTGAAGGTGCTGTCCATGGTGAAGCAGTCAGAGCTGTTCGA
CAGGTGGAAGAGCCTCCAGATGTGCAAATGGGCGATGAACATCTCTGAGGCCAACCAGTTCAAGTCTACTCTG
TCCAGGTGCTGCAACGCCCCTGCCTTTCTCTTCACCACCCAGAAGAACACTCCCCTGGGGACAAAGCTCAAGT
ATGAGGTGGACACCAGTGGCATCTACCACATCAACCAGGAGATCTTCCGCATGTTTCCCAAGGACATGCCCTA
CTACCGGTCCCAGTTTAAGAAGTGTGCTGTAGTGGGCAACGGAGGCATCTTGAAGAACAGCCGCTGCGGGAGG
GAGATCAACAGCGCCGACTTCGTCTTCCGGTGCAACCTGCCCCCCATCTCAGAGAAGTACACCATGGATGTGG
GGGTGAAGACGGATGTGGTCACTGTGAACCCCAGCATCATCACAGAGAGGTTCCACAAGCTGGAGAAGTGGCG
GCGGCCGTTCTATCGCGTGCTGCAGGTGTACGAGAACGCGTCGGTGCTGCTGCCTGCCTTCTACAACACGCGC
AACACCGACGTGTCCATCCGCGTCAAGTACGTGCTGGACGACTTCGAATCGCCGCAAGCTGTCTACTACTTCC
ATCCGCAGTACCTGGTCAACGTGTCGCGCTACTGGCTCAGCCTGGGGGTGCGCGCCAAGCGCATCAGCACCGG
CCTCATTCTGGTCACTGCGGCGCTGGAGCTCTGTGAGGAGGTGCACCTCTTTGGCTTCTGGGCCTTCCCCATG
AACCCCTCGGGCCTCTACATCACTCACCACTACTATGACAACGTCAAGCCGCGTCCCGGCTTCCACGCCATGC
CCTCTGAGATCTTCAACTTCCTGCACTTGCACAGCCGAGGCATCCTCCGCGTGCACACGGGCACCTGCAGCTG
CTGCTGATGGCTGCCAGCCAGGCTGCCCGGCAAGCGGCGCCCCCTCTCCTGCTGTCCCTCCTGGTGGAACTGG
GAGCCCCGAACCGGGCAGCGTGGGGTCCTGGCGTTCAGGCTGTCTCTCCCTCCATTGTTCAGCTCTGTACTTG
GATCCTGGGGTAGGGAGGCAGGGTTAGGAACCGGGGCAGAGTCAGCTCTGTGCTGAGCCTCCTGGCCTGGCCC
CCACCGGTGCATCCGCCCAGTGCGTCCACCTGCCCTGGCTAGCATGCTGCTGGGCCACACCCCAAGATCAGGG
GCCCTGGGGACGGCAAGTGAATAAAGCACATTTCCACCCAATTTTGTCATCCGAGAGAGAGCACAAACTGCAG
GCCCTTGTTGCAGCTGAAGGAAGACCCTACAGAGAATGGAATTGAGAGTGGAGACAGGACACTTCACAGGACA
CTTGAGCACAGTCAAGATTTTATTCACACTTTTGGTTCCTGTGTTTTATATCGAAGACTTAGCTATGAATTGC
TATCTAAATCTCAGAGCTTAGAAGCCAACCCAGTGACTAGACCTTCCAGTGAAGAAAGTGATCTCAAGAGGTC
CAGGGACTTAACAGCCAAGCCACACCACCCGACAGGTTCTTCTGTGACACCGAGAGATCTAACCCAAGGCCTG
GGTTATGTCTTAGTCGAGACATCATCATCTGAGAAGCACCACATCCTTTCAGACACATCCTCGGACACACATG
CGGTCTTGGTGCCCCCAGGTTCAATCCTGCGTTAGCTCTTTTGATGAGAAGGAAATAAACCAAACCAGCCACT
TGCACTATGGCTTCCAAGCCAATGTTATTGACTCAATAAGTGCTTAGCAATTGGCTTCCTCTTTTGTCTTCCT
ATTTTCAGCTCCATTTAAACCAGGAGTTATTTATAAAGACCTCTTCCTCTCATGTCTCTGAATCCTGCAGAAT
CCTGCTGTATCGTGTTCCTCAGTCGGAACCATAGGCTCTGGGCTGGAAAGCACTGACATGTCAGGTGGTGAGC
TTAGCAGAGAGAAACGAGTTGTGGCCTCCGACACCAGCATCACTGAGGCTTCCGCTGTGAAGGCGGGACTTCT
CACAGCAGACCCCAGGGCCTGGGCTTGGGGCAGTCAGCTGGGCTTAGGCAGAGAGGGGTTCACATATCATTTC
ACATCTGAAATTAATTCCCTTTGCTTCTCCCTGTGGTGTTTCCACCATAAATGGGATGGTGTCTAAGAGACAC
TGTTGGTGAACTAACTGCCTTAGAACTTAGCTTTGTGGAATCATAGGATGTTAGTGCTGGGAGGAGGACTTCA
GCCTACCTGCCTGGCAACCTGTCTCCCTCCCTCCTTCCTTTCTCCCTGCCTCCTCCCCTTTCTCCTTCCCTCC
CTTCCTCTTTTCTTCCTCCCCTTAAACATTTCTGGAAAGCCTATTCTGCTTTGGTCTGTGGTTGGACTTAGGA
AGACATAAGGATAAACAAAGGCATTATCTGTGTCTTCTGGGGGTGCAGGGCCCAGAGATTATAGACCCAGTCC
CACGTGCTGGGTGCTCTGGGAGGGTTAGGAGCTGTAAGAGTCTATCAGGGAGTGGGATCTGCTATCCAATCAC
CTCATTTGACAGGCCCCCCGGCTGAGCCAGTCAGGGACGTGGAGAGTGCAGGCTTTATCCCAGGCCTCCAGGC
TCCTGATCCAGGGCTCCGTTCTTGGCCAAACTTTTGTGTCTTGGCTTGGGCTTCCTGTGGGGGGGTTGTTGTG
GAACCTGTGCCCTGTGCACAACACTGTTCTCAGTGCTGACTCTCACCTGCATTCTGAGCTCTGGTCCTGGTTG
GGAAAACCTCAGGTGGCTCTGAGTCTCAGTCTGCTGGTCAGGAATGTAGGGGAGTAGCCAAGGGGATGAAATG
ATCCCAAACACCTGCTAAACCAGGAAGAACTGCAAAGGACAGTGGATGGTTATTACTTGCTTGAGCTCTGCCT
ATTTCTCCCAGGGAGGGAATTTCTGGGAAAGATTCCAGTTACCTGAGATTTCTAGCCACTGCATGGTTAATTG
AACCCAGTTCTTGAAACCATGGAATATAAAAACTAGAAGGTTCTTAAAGACCATTTTGTTCCACTTTAGGAAA
CAGGCCCAGAGCAAAGATGTGACTGGGTCAAGTCATGCCTGGGGCTCTACTGAGTGCCTGCTGTGGACAGAGC
CCTCTACTGGAAGCCCTCTGGGTACTAGTTATGTTCCAGATGGGCAGTGTTTGGGGGAGGCTCCCTGGTGACA
CAGACATCAGGATTCCCACAGCTGAGGGTGTTGCAGGTTGGAATCTGCCCCAGCCAGGGCCCTCGTAGGAGCT
GAGGATTGGAGCATCGGGATCCAGCCCACAGAGGAGTTGGGACACCTGGCTGCTGGCCATATTCCTGCCTGTG
GGTCTTAGGGCCAGTCCTTCCTCTCCCTCAATGTCTCAGTTTCCTCATCTCTACAACTAGAAGCTTTGTAAAA
ATCTTCAGCGTCTGTACAATGTCCTTATTTTGGGGGGACATTCATGATGAATGGATCTAAACCATAATTAATA
TTTCTCATTATACTAGCTAGCATTTATCAAGCTACGTTAAGCACTTTCCCTATATTATCTCCTGTAATCATCA
TGAGACCCCTTTGGGTAAGGAGGCTCCCATTGTACAGATGAGGAGACAGAGGCTTAAAGCAATTCCAGGACTT
GCCCTTGGTCAGGCACCCAATGGAGGGAACGGCACCCAATGGAGGGAACTGCACCCAGATCTGTAGAGCCCCA
AAGTCTGTGCTCCAAACCTCTGTCTTAGCTTCCCAGACAGTGACACCAGAACATGAGGGATCCTGACCAAGAT
GCCCCTGCAGGATAGTACGGGGCCCTCCTCCAGGGACAAATGCTCTAGGAGGCTGTCATCGTCCGGGAAGCAG
CCTGTGCTTATAGGTGCCTCTCCCTAACCCTGCCGCCTACAGCAGTAGCGACTTACCCACAGTGGCACTGGAG
GGGAACTGAGGCCATCGTCACATTTTTAAGGTGAGGAAAGATGCCCAGAGATAGCACATGTTAAGTGACAACA
GAATGACTTGCCCAGGGCTACATGGCCAGCTTGTGGCTCAACTGGGACTCACCAGTCTCCTGACTTTCAGGA
TATAGTTTTTCTGCTGCAGCCAGTGTTTATAAGAAAAACATTTTTTCAAAGGCTCAGAGAGACCTGGTGTTGA
CTCTGGAAGCCTTCTGCCATGTTCCTCCACAGCCCTGTCACCACGCCACCATGCAGTGCAGACTACCCATTGA
ATTATTATTCAAGGGAAATCGATTCCCGTAAACAAAACAACCATCATGTCTTCCTTTGTGCTAAACCCACTGC
TCCAGGCTTGAAAGGCAGCAAAAACAAGTAAATGACTCAGTGCTTAGCCAAGCACAAAGAAAGGTGGCTCAAG
GTCAGAGGCAGCGTCTGAGACCATTTCAGCAATGCTGCTGCGGCAGCTCTGCCCACTAATCACCTGGTGCCCA
GAGACAGCATGTGCTCATCGCCCTTGACTTTGCTGAGAGCAGGGAGGAAGCAGAGCAGTGCTTAGAAACAACT
GAGGCAGAGTAGAGTGCTGGCCAATTTAGTGAGTGAATTGGTGAATAAATTATATTTATTGAGTGCTCATGTT
GTGCCAGGCATGGTGCCAGGTGCCGGGGAGCTAGTGATGAACAAAACGTCGTCCCTGCCCTCCGGGAGTTTGT
GGTCTGGTGTGTGTGTATACAGACGTTAAATGACGAATCCCACAAACGTACGTGGGAGCTGGTTGTGCTGAAC
ACTATCAAATCCAGTCTCAGGGACAGTTATCTGGGAGAGGTGGAGGAGGGGATGACCAAAGTCAATTCCGAAT
TCTGAAGGTTGGCAGCATAAAAAGTGTAATGAGCAAAGCAGCAGATACTTCTCAGATATCATCCTTGACATCC
GTGTAACATTTGACAAGGTTGGCCTCTCTGGTCTTTTTTGTTTGTTTGTTTGAGACAGAGTCTCCCTCTGTCA
CCCAGGCTAGAGGGTGGTGATATAAATATGGCTCACTGCAGCCTCGACACCTGGGTTCAAGTGATCCTCCCAC
CTCAGCTTCCCAAGTAGCTAGGACCACAGGCACGTGCCATTGCTCCCAGCTAATTTGAAAATTTTTTTTTTTT
TGTAGAAACGGGGTTTTGCTATGTTGACGAGGCTAGTCTCGAACTCCTGAGCTCAAGCAATCCTCCTGCCTTG
GCCTCCCAAAGTGCTGGGATTACAGGTGTGAGCCACTATGCACAGTCTCTCTGGTCTTTCTAAAGTCCTCTCC
TCTTCTGTTTTCTAACTATTCTTCCCTGACTTTCCTCCTGTCTCTCTGAATGCTCCTTCTCAGGTTCCTTTGT
GGATTCATCTTCCGTGAAGCTTCCTCAGGGTTCTCTTCTCTTCCTAGGAGCATTCCTGCAGAAATCTCATCTA
TTCAAAGCCTTAAGTATCTTCTGCCCTGGCATCTTAACCCCTCTACCTGCAGGCCAGTCCTCCCTAGGAGCAT
TGGAAGCATGCTCACTGTGGCCTCCTGCATGTCTGTGTCTGCACAGCATCTCAGACCTCAACATGTCCTCAAC
TGGTATAGTTAAAAGAACAATCTGAACTCAGAGTCTCCTCCTACTACTTCGTAGCTGTGTGAAATTGAGGGAG
CTCCAGAAGCCCCTGGAGGCTCTGGACTCATCATCTGAAATGAGGACAGGATACCAACCACAACAGCTGTTGC
AAAGCTAAGTGAAATAGTGTTGCACAATGCCTAGCACAGTGCCCAGCACAAACTAGACACTAGCAAGCACAGG
TTGCCTTGGCCCAAAACTTGGATCACCTTTGCCTCTTCCTTCTCCTTCACTCTTTATGTCTATTTGTTCACTG
TATCTTGTCAATTCCAACTCATAAACATCCCTTTAAGTCATTCCCTCCCTGCAACCCCTCTGCTACTGCCCTA
ACCCAGACCATCTGATGGATCTTTTTGCTTTCAGAGAATAACCTTCCAAAAATACCTTTTGTGGGGCCCATTG
CCTACCTGTATCATAAACTTCAGGTTCTTTACCAAGTCATGCAGGGAATGTCCCAAGCTGGCTTACTCACCTT
CCCAGAGATCCTCCTGCCACCTCCCTGCCCTATCCCATACTATTCCAGGCACCTTTGCACACTCTGTTCAAAC
ACCTGGAATGGCCCTCACACTCCTAAGTTCCTCTCTCCTTCCTTTTATCTGGCCACTTTTGCAGAAGTCAGGT
AATGGCCCCCTGCATCCTCCCCTCTAGAGACCTGGGCTGGGCACTCTCTCCTCTGTGCTTCTGCTGCAACCAT
CACCGACTCCATTAGGACATCCTCACAGCACACAGCATAAGGGCCCGTGTGTCCTTGATAGAAGGTTTGCCTT
CTCCCCAAGGGAAGACACGATATCTAAGCTCATTTTGTATTGCCAGCACTGGCGGGGAAGCACATGCCATGGC
CACTCAACACATGTTTACTACAGTAATGAGGTCTCTGGTTCCTGGGGGACTCCAGCAGTTCAGGATACCCCGC
TAATAAACTGAGTAACACCTTGGGGAAATTGTCAGCTGAGGAGAGTCCTCTATTGATTTTTTTACCTAGCACA
TCCCAGGAAGGCAGAGACTCCTCCTGCCTGTCCAGGGGAGCTCGGGGCTCAGGAGGCTGAAATGTCTGTCTAG
TGGGGGCTTATGGGCTTGGCTAGCCTCTTTTTGTTTTCCCTCATTATAATCCTGCTCATCTGCTTTTTGCTGG
CTAGCCAATCCTTTTTCCTCTAGTTTAAATGAAAGAAGGGAGAGACATTTTCTGTGTTTCTTGTTGTCTACTT
CTTCCCTTCCTCAAAGCCACACAAACATGTCAGGCTCTTACATCACTGTGGCTGAGGTGCAGCCTGGGAAGCA
ATGTAAGAGGGATTTATCAATGGTGTTTAAAAAACTAGAGGAACATGCTGAATGCTTTGGCTGGGAAACAGGT
GAGCTGAGCTTTGCAGGATGTCTGCTCCAGATGCGAAGGGGAAGACCAGGAGCATGGGGTTGTTTCCTGGGGG
AAAAAAGGTGCTGCTTCTCTGAGCTCAACACACGCCCTGACCTAGTCTCCTCCCTGTAACCAGAGCACCCTCT
GGGAAGAGGCTAACTGTGGCTCAGGGGTCAGGTGAGGCCAGGCGAGGGGTGCACCAGTGGCCAGGGTGTGCCT
TGTGCTATTGTTTAGAGGTCTAGTCAAACGAGGCCAGTCTGTGGAGGCTGATTCCATCCTACGGGGCATCCTA
ATAAACCAGTGTGATCTTCAAAGGGTAGACTGGATAGATCTACAGTGTGATCTTCAAAGGGTAGACTGGATAG
ACCTACAGTATAGATCTACAGTATAGATCTACAGTCTACCCTTTGAAGATCACATGGGTTTATTAGGATGCCC
TGTAGGATGGCCTTCTGATGGCCTGCATCAGAATCACCTGGGTGCTTGTTAAAACTATGGACTGAGAACCTGG
GGGAGAGGTGGCGCCTGGGAATCAGCATGTGTGAGGCCCTATCTGTTCCCTGAGCATACAACTGCTTGTGGTT
CAGAAGTTGCCACCAGTTGAGTGGCCATGTATGGCTTGAAATTCAGACTGGAGCCTTGAGTAGGAAAAGAATG
AAAAGAAGAATTTGGCACACAACCCAACTTCTGCCTTTTCTGTACATACAAATATAATATATAACCCACTATG
TACCTAGCAACAAAAGTCAAGTGCTCGGGAATCTAACATTTACAAAATTGGTTTTGCTTTAACACAAACAAAG
ATTTCTTTTTCATTTGTAATTATGGTAAAATACACATAACATAGAATTTATCATCTTAACCATTTTTAAGTGT
ACAGTTCAGTAATGGTAAGTACATTCACACTGTTGTATATTCTGTCTCTGGAACTCTTCTCATCTTGCAAAAT
GAAAACTTTAAACCCATTAAATGACAAGTCTTCACTTCCCCCTTCCCCAGCCCCTGGCAATCGCAATTCTACT
TTCTTTCTCTATGAATTTTACTGCTCTAGGTCCCTCATATAAGTAGAATCATATAATATTTGTCTTTCTGATG
ACTGGCTTATTTCACTTAGTATAGTATACCAGAGACTTTTTAGAATCACTTAGGTGCAAGCAGCTCTCAAATG
ACATTAACTGTTTTGGGTGTCCCTAAAGCAGAAATCTGATTTTTGTTTTTGTTTTTGCCTTGGGCAGTTGTCC
TGGGCTGGTCGTTGGCCCCTGGCTTCCTGAGACCTTGGCCCCTTGCTGAGGGAGAGTGCAGACCTGGCCTTGC
TCTGCCACCTGGGCTCCACTGTCCTGGCCAGCATGTCCTGAGAGACAATAGCCTTGCTGATCTTTTAGAAGTC
TTTAGGTATTTTCCCCTGCACAGTCTCTTCTGAGAACCTTTCTTTTTTTTTTTTTTTATTTCAAAGGAAAAAA
ACCAAAAGCAAAAACATACACTTCCATGACTTAGCTGTTGGCCACAAAAGCCAGAAATCAGTGACTCTCTCTT
CAGTTGAAGATGGTTCCCATGGCAACAAATCTCTCATTTCCTTCAACTAAATGATCTGCTCAGCTTTGGGGTA
AAAGTTCTGTTTTCTCTAGAATTGCCATTTGCAACCTTGTGTGTACATAGGAATCACCTGGAAGTCTTTATAA
AATAATCCCCAAGCCAGGTCCTTTACTTCCCGGAGGATCTGATTCAGCAGGCCTGGGGATAGGGCCTGGTATC
CCTATTTTGAAAGATCTCATTAAGTGGTTCTGTCTCACATGGTTGGGAGTCATTACCCTAAGGCAGGCAGTCT
ATGCCTTGGTGCACGCTAGAGTCGTCTGAAGAGCTTTAAGAAATACTCGTGCGACCCCACCCCTTGACATTCT
GAGTCAGGTGGTCTGAGATGTGACCTGAATATTAGGATTTCACAAATTCTCCAGATGTTTCTAATGTGCAGCC
AGCCTTGAGAACCACTATTCTAGGTCAGCAGTTCTCAAACTGGAGGCTGCCTCAGAATCTCTTGGAGAGCTCC
TTGATTATGGGCCCCGCCCCAGAGTTATTTGGTGAGGCCAGGGTGGAGCATCTCTAACAAGTTCCCAGGTGAT
GCCAATGCTGGTCCAGGGACTGCACTTGGAGGCTCATGGCTCTAGATGATCCTTTAATGAGATTGGCCCTGTT
CTGCTGCCCTGTTCTTTTTCCTGGTTGGGGGTTGGTGGACCCTTCCTTCCCTCAGTCTGACTCAGGCGTGAGA
CTTCTTAAAGGTACCAGCTTTTCTTTTTCTGAACATTTTAGCATTCAGATAAATATTATGACATATTTTTAAA
GCAAAGTAACATAGATTTCTTCTCCCGGGGCTAGGAGCCTCAGAGTCTGCCTGAATTAAGAATTTATCTGTGC
CTAAGATTAGGATGCATTTAGTGATCTGAGCTAAGGGGTTGGTGGATCTGCAGCAATAGGTTCAGCCTGTGCC
TGGGTGAGGGTCACCTTGTGACCGGCGCTGGGTAGGATCTGAGTACCACACACTTGGCCCTCTAGCATGGGGA
GGACCTGTCTGGTCCACCTGGGGGAAAGCCTGGTTTTCTGATGCAGACCCAGCCCTCTGCCCACCTCCACATC
TGTGCACAAACATGGAAGGTAGCAATGAGGAAACACAGCACCAGGACTGCCGTGAGCCCAGGAGCTAGGGTTA
GAGCCTCACTTCTTTATGCATTTCCTTTTCTATCATACTTTGGAACAGGCTTGAGCTGAAACCAGAGAGTTAG
TACCTGGTTTCTGCCTCAATCTAGTCTCCCTCCTCCAAAGATGCCCCCACAACGTGGGCAACTTCAACCCCCA
TGAGAGCTCAGAATTCCACATTCAGCGACAAAGTTGACAAGAGCCTCTGGAGATCTCAAAGACTTATTAAAAG
TGGAAATGGCTACTGTTTTACTGAGGCAGGGTGAAAACACAACTCACTTCCTAGGATCGAACCCAGCAAATTC
ACTCAGCCCCCAGGTAACTGAGGACTCAGTTGAAGGAAAATAATTTTGAGCCCCTGAAAATAGATGCTATAAA
GCTGTGTATCAAGGGCCAACCATTTTGCTAATATCACATATCTCACGGCATTTGCTTTGATTTTAAATTCAGT
TCTAGTGAACTTGGTTCTGATTTCTAATGACAAAGCAAACTAAGAGAATGATGACATGTGCTAGATAAAGCAA
GAAGTGGCAGCTAATAGCCTAAATTGTGATGGCATTAAGAAGACCAAAACTAGTTTACTTGTTCAACGACAGC
TTGTCTTTGGAAAAAAAAAAACACTACATTAAGGATAAGAAAATATGTCCAGGCATGGTGGCTCACATCTGTA
ATCCCAGCATTTTGGGAGGCTGAGGCGGGAGATCGCTTGAGCTCAAGAGTTGACGACCAACCTGGGCAACATG
GCAAAACCCTGTCTCAATAAAGAATACAAAAGCACACCTGTAGTCAGTCCCAGCTACTCAAGAGGTTGAGGTG
GGAGACTGGCTTGAGCCCAGGAGGCAGAGGTTGCAGTGAGCTGAGATCATACCACTACACTCCAGCCTGGGTG
ACAGAGCCAGACCCTGTCTCAAAATAATAATTAAAAAAGACCAAAACTAAATACATATCTCAGGAACCATCAA
GTAGCCTGAGGTCGTTGGTCGTTTAGTGGGGAAGAGAGGGAGAATATGTTATAATAACCCCGGGAGCAACACA
GCATTTCAGCTCAGTTCACCCTGTGACATCCAGGACATGCAGAGGTAAAAAAGCGTTCCAACAATGTTCAATA
CACTGTCTAAGAAGGCAGGGCTGCGTTTTATCCACTTTAGAATTATTTCCATCCCCAAACAGTTAAAATACTT
TCACAAAAGTCTTGGTGCTTAAAAGATAAATGCTCAGTGACCTGGAAGTTCACATAAATGAAACGGTTGATGA
TCTGATGATGAAGGATAGGCGAGATGGGTAAACTCACTCTTTTTTTTTTTTTGAGATGGAGTCTCACTCTGTC
ACCCAGGCTGGAGTGCAGTGGCATGATCTCGGCTCACGGCAACCTCCGCCTCCCAGGTTCAAGTGATTCTCCT
GCCTCAGCCTCCCAAGTAGCTGGGATTACAGGCGCCCACCACTTTGCCCAGCTAATTTTTGGTATTTTTAGTA
GAGACGGGGCCACCATTTTGGCCAGGCTGGTTGTGAACTCCTGACCTCGTGATTCGCCCACCTCCTCCTCCCA
AAGTGCTGGGATTATAGGTGTGGGCCACCGTGCCCGGCCAAACTCACTCTTTTGAACTTAACATTTTCCCCCT
GACATTGGAGACTCTCCTGCTACCTCTCTCCTAAGAAAAAAGGGCAGGTGGTGGGGGGACAAGCCCCTTCCAC
CTTCCCATCTCCAAGGGACTTTGCAGTGTGGGTGATGGTGTGGCTGATCATCCTCTTTCTGCACCTCTGATTC
TGCTTGTGTTGTCCTTATTGTGTGGGGCAGGAGGGTCAGGACTTCAGCTAGGGGTGTGCTGGCTCTGCAGTGA
CTCAGGCCTGGGTGTTTTTTGGTGAATTTAAAAAGATGACCATTTCTGGGGGAAAGTGACACTTTTACTTTCT
CCACACCTTCACCATCCATTTTCTTACTGGACTTCGCTGCGTTCCTGCAAGGAGGACAGGGCAGAGAGTAGCA
ACCCTCCTTGCCGAGGCCCAGAAAGATAGCGTGGTGTGCCCAGGGCACCCATGGTGGGTGATGGACATCTCCT
TTTGGGTGCCAAGGCTTCCTCAAGGTGGGAGGAACTGGAGGGCAGGGGACGGTGGCCAGTGTCCCGCTCTAGC
CCTCTAGGGCACCTTTTTCTCCCCTCTGAAGTTGTTAGGCTGATCTGGAGTCTGCTCCAGGCCTGGGCTTCAC
CCTGGCCTCATGCCCTGCCCAGCACCTGTGTGGAGGGACCTGTTCGTATTCATTCCTCTTTGGGGGCCAAGCT
CAGGGCACCCGAACCAGCAGCCTTGGGTGGCTGAACCTAGTGGGACAGAAGGGAGGCAGTTGTATCTGGCAGA
GTCACCCAAGGCCACAGGCCTGAGCAAAGCAGGAGAGCGGGGCAAGCTGGGGTCAGGGACCATGGTGCTGGCC
CCCTGCTGCTCTGGCCATGTGGGAGGCAGCTGGAGGGGTCCCAAGTGGCAGCTCCTGCCAGACTACCCCGGAC
TCCCGAGCCCATCGTCCTGTACTCTTCTCCAGTGGGACGTCTGGCCCTCACTGTGGCTAGCTTCTGGGCAAGT
CCCTCTGCTCAAGATCACTTTATGGGGCAGCTGGTGCCCCCTGGGGCTCCCACACTCTGCACATACAATTTAG
GGACCTGGCAGGGTCTTGGTGGCCTGCAGCTTCCCTTTCTGGAACCGACTCGCCTCCCATCAGAGAGCCATAA
CTCTTCTTCAATACAGTATTGTTTGAACTTTGAAATATATGCATTTGTCTATGTATTTTCTTAAAAATTAACA
CTGTATCTCAATAAGCATACAATGGATTGTTCAATGGTACAGTTTGGAACCAATCCCCCGCCACTTCCCTCCC
CAAGTAAAGCCAGGACAGATGCTGGAGTCAGCATGGGAGCTGTGGAAACCTTTTGCCCTGGAGCCAGGTGCCT
GTGGCTGTGCCTGGGAGTGGCTTCAGCCCAGGCGTGGTGTCAGTTTCCTGGCACCCCTGCCCAGGAGCCTGCA
TGAAGGCAGCCGGGTCTCTCTCTGAGTGGCTGGTGCACCGGCTCATTTCTGAGTGGCCTCCTGCGTATGAAGA
AAAAGCGGCCACGTGGCATGCCTCCCTTCCCTCCCTTGCTTTCCTCTCAAAGAGCCAAGTCACGCTGCACTGC
GCTCCGTGGAGCCTTTCCCGATGGAGAAGAGAAGGGAGGCCTGAGATGTCCTTTGCTGGGATGTGGAGAAACA
CCATCTATATGAAAAGCCCCTTGCTTGCTCTGCTGATTTTCTCCCTCCCCTGGCCCTGGAGAGAAAGGCTGTG
CCATTCAAAGACAGTTTGGGGACATGGGGGCTGCAGGCCTGATCTTATTTTTATGCAAATTTTGTTCTGGGAG
CAGTGGTTTATACAAACTCCTGTAGCATAATTCCAGCTTGTGTGTGGCTGTCCTTGAGTAGTGATTCTGAAGT
GATGACTGTGATGCTATGCCTAAAAACATCATCAGGGTACTTTGTATCTGTCACTGTGTTTGAACGGCCCTCA
ATTTCTATCTAATAAAACCAGTCAAATGGTTCACCTA
  (SEQ ID NO: 197)
MRYADPSANRDLLGSRTLLFIFICAFALVTLLQQILYGRNYIKRGLQFGWQSGDQLTNWTGLENVTDDPAVQS
GSDSSSRYFEFYEGPFEYNSTRCLELRHEILEVKVLSMVKQSELFDRWKSLQMCKWAMNISEANQFKSTLSRC
CNAPAFLFTTQKNTPLGTKLKYEVDTSGIYHINQEIFRMFPKDMPYYRSQFKKCAVVGNGGILKNSRCGREIN
SADFVFRCNLPPISEKYTMDVGVKTDVVTVNPSIITERFHKLEKWRRPFYRVLQVYENASVLLPAFYNTRNTD
VSIRVKYVLDDFESPQAVYYFHPQYLVNVSRYWLSLGVRAKRISTGLILVTAALELCEEVHLFGFWAFPMNPS
GLYITHHYYDNVKPRPGFHAMPSEIFNFLHLHSRGILRVHTGTCSCC*
```

An amino acid sequence for the protein encoded by the TNIP1 gene that is a regulator of T cell exhaustion/dysfunction resistance is available from the UniPROT database at accession no. Q15025, cDNA NM_001252385, with gene location at Chr 5: 151029945-151093577.

```
  (SEQ ID NO: 198)
AGTCTCCGGGGACTTTCCCAGGGGTGGGGCGGCCCGGCCAGGCCCCCGGCACTTCCTCGTCCTCGGCCCGGGT
GCCCTGCCCCCGTCCAGGAGCCCTAGGAGTGCTACGGGGGGCCGGAGCCTTGCCCGGGCCGCTGCCCCGTCCC
TGGATTCGGGGCTGGACGCAGCAAGCGGGGCGCTGTGTCCCCAAGCTCCCCGTCCTCGGCCAGGCGGGCACCA
CGGCAGGGGCTGAGCTACCCTCATGGAAGGGAGAGGACCGTACCGGATCTACGACCCTGGGGGCAGCGTGCCC
TCAGGAGAGGCATCCGCAGCTTTTGAGCGCCTAGTGAAGGAGAATTCCCGGCTGAAGGAAAAAATGCAAGGGA
TAAAGATGTTAGGGGAGCTTTTGGAAGAGTCCCAGATGGAAGCGACCAGGCTCCGGCAGAAGGCAGAGGAGCT
AGTGAAGGACAACGAGCTGCTCCCACCACCTTCTCCCTCCTTGGGCTCCTTCGACCCCCTGGCTGAGCTCACA
GGAAAGGACTCAAATGTCACAGCATCTCCCACAGCCCCTGCATGCCCCAGTGACAAGCCAGCACCAGTCCAGA
AGCCTCCATCCAGTGGCACCTCCTCTGAATTTGAAGTGGTCACTCCTGAGGAGCAGAATTCACCAGAGAGCAG
CAGCCATGCCAATGCGATGGCGCTGGGCCCCCTGCCCCGTGAGGACGGCAACCTGATGCTGCACCTGCAGCGC
CTGGAGACCACGCTGAGTGTGTGTGCCGAGGAGCCGGACCACGGCCAGCTCTTCACCCACCTGGGCCGCATGG
CCCTGGAGTTCAACCGACTGGCATCCAAGGTGCACAAGAATGAGCAGCGCACCTCCATTCTGCAGACCCTGTG
TGAGCAGCTTCGGAAGGAGAACGAGGCTCTGAAGGCCAAGTTGGATAAGGGCCTGGAACAGCGGGATCAGGCT
GCCGAGAGGCTGCGGGAGGAAAATTTGGAGCTCAAGAAGTTGTTGATGAGCAATGGCAACAAAGAGGGTGCGT
CTGGGCGGCCAGGCTCACCGAAGATGGAAGGGACAGGCAAGAAGGCAGTGGCTGGACAGCAGCAGGCTAGTGT
GACGGCAGGTAAGGTCCCAGAGGTGGTGGCCTTGGGCGCAGCCGAGAAGAAGGTGAAGATGCTGGAGCAGCAG
CGCAGTGAGCTGCTGGAAGTGAACAAGCAGTGGGACCAGCATTTCCGGTCCATGAAGCAGCAGTATGAGCAGA
AGATCACTGAGCTGCGTCAGAAGCTGGCTGATTTGCAGAAGCAGGTGACTGACCTGGAGGCCGAGCGGGAGCA
GAAGCAGCGTGACTTTGACCGCAAGCTCCTCCTGGCCAAGTCCAAGATTGAAATGGAGGAGACCGACAAGGAG
CAGCTGACAGCAGAGGCCAAGGAGCTGCGCCAAAAGGTCAAGTACCTGCAGGATCAGCTGAGCCCACTCACCC
GACAGCGTGAGTACCAGGAAAAGGAGATCCAGCGGCTCAACAAGGCCCTGGAGGAAGCACTGAGCATCCAAAC
CCCGCCATCATCTCCACCAACAGCATTTGGGAGCCCAGAAGGAGCAGGGGCCCTCCTAAGGAAACAGGAGCTG
GTCACGCAGAATGAGTTGCTGAAACAGCAGGTGAAGATCTTCGAGGAGGACTTCCAGAGGGAGCGCAGTGATC
GTGAGCGCATGAATGAGGAGAAGGAAGAGCTGAAGAAGCAAGTGGAGAAGCTGCAGGCCCAGGTCACCCTGTC
AAATGCCCAGCTAAAAGCATTCAAAGATGAGGAGAAGGCAAGAGAAGCCCTCAGACAGCAGAAGAGGAAAGCA
AAGGCCTCAGGAGAGCGTTACCATGTGGAGCCCCACCCAGAACATCTCTGCGGGGCCTACCCCTACGCCTACC
CGCCCATGCCAGCCATGGTGCCACACCATGGCTTCGAGGACTGGTCCCAGATCCGCTACCCCCCTCCCCCCAT
GGCCATGGAGCACCCGCCCCCACTCCCCAACTCGCGCCTCTTCCATCTGCACCAGTTTTGTCGCAGCCGGAAT
ACACCTGGCGTCTACCCTGTGGAGGGGTTCGAAATCCAAATCAGAGCTCCCAAGTGATGGACCCTCCCACAGC
CAGGCCTACAGAACCAGAGTCTCCAAAAAATGACCGTGAGGGGCCTCAGTGAGACCAGATTGTGTCATTTGGC
TCCACCTTCATCTTGCAGAGCCAGCTGATCTCAGATTGCCAAGAAACTAGAAGCCACTTGCACGGTGTGGCCA
GAGCCTCAGCTGGATGAGAGGCTGAGATGGGTGGCCAGCTTGTACACCAGTCCCTGAACTGAGCTGTTTACAG
GACTGGGGAGGCTCCACCCAGAAGGCTTTCATTTGTACTCTGCTGGGAGTGACTGGGAAAAACTCCTTCCCTG
CTGCTGAGTGGAGAGAGGCCTCATCCGGCTTTGACCCACCATCCGTTGCAGAAGCCTCCAGGAGCAGCAATCC
TAAGAGTGGGAGGCAGCCAAGACCCCCTTCCTTCAAAACCTCCCGGAAGTGGTTTCAGGCCCTCTAGTTGCCA
TGACCAATTTGTGTGTGTGTTTAATTTTTGCTTCAAGCTCTGTAGCAGGACCTGCCCCACGCACACCCCTACC
CCTCTGTGAGGAGCTGTGGGAAGTGTGGGTTTGTCTCCAGAACAGAAGAGAATGATGGATATTCTGGCTCTGG
GGCCCTCTCCACCACCACTCACAGTAGCCTTGCTGAAGCCATCACAGATGGGAGAAGGCCATGCCAGCCACGT
CCGCCGAGGGGCGCCAGCCTGAAGCTGCCAGGCCCTGAGGTTCAGACCCTGGACCCCATAGCTGGAGGCCTGT
GGTGCCAGAAGCCCAGATTAGGGTGGCTGTCCATCCCTGGATAGCTATTTGCACGAATCATGGACATAAATCC
AAGTTGAAGAAGATC
  (SEQ ID NO: 199)
MEGRGPYRIYDPGGSVPSGEASAAFERLVKENSRLKEKMQGIKMLGELLEESQMEATRLRQKAEELVKDNELL
PPPSPSLGSFDPLAELTGKDSNVTASPTAPACPSDKPAPVQKPPSSGTSSEFEVVTPEEQNSPESSSHANAMA
LGPLPREDGNLMLHLQRLETTLSVCAEEPDHGQLFTHLGRMALEFNRLASKVHKNEQRTSILQTLCEQLRKEN
EALKAKLDKGLEQRDQAAERLREENLELKKLLMSNGNKEGASGRPGSPKMEGTGKKAVAGQQQASVTAGKVPE
VVALGAAEKKVKMLEQQRSELLEVNKQWDQHFRSMKQQYEQKITELRQKLADLQKQVTDLEAEREQKQRDEDR
KLLLAKSKIEMEETDKEQLTAEAKELRQKVKYLQDQLSPLTRQREYQEKEIQRLNKALEEALSIQTPPSSPPT
AFGSPEGAGALLRKQELVTQNELLKQQVKIFEEDFQRERSDRERMNEEKEELKKQVEKLQAQVTLSNAQLKAF
KDEEKAREALRQQKRKAKASGERYHVEPHPEHLCGAYPYAYPPMPAMVPHHGFEDWSQIRYPPPPMAMEHPPP
LPNSRLFHLPEYTWRLPCGGVRNPNQSSQVMDPPTARPTEPESPKNDREGPQ*
```

An amino acid sequence for the protein encoded by the TRIM56 gene that is a regulator of T cell exhaustion/dysfunction resistance is available from the UniPROT database at accession no. Q9BRZ2, cDNA NM 030961, with gene location at Chr 7: 101085481-101097967.

```
  (SEQ ID NO: 200)
AGTTTTGTTTCTTTTCCAGGCACCCAGCAACGGCGGCCTCCAGGCCTCAGGCCCCCTCACCATCCTAGAGGTC
AAGATCAGCTCTGGCTAGTTCTCACAGGTCTGACCCAACAAGTAGCACTGACATTTTTACGTTTGCTGGATGT
ACACACGGAAGTGGAGGAGGAGGAGGAGAAGGAGGAGGGCAGCTCCTTAGCTCAAGAGCAAGTGGCCCAAGGC
CTCAGAAGACTAGAAGGAAGTTCCTGGCCATTCAGCATGGTTTCCCACGGGTCCTCGCCCTCCCTCCTGGAGG
CCCTGAGCAGCGACTTCCTGGCCTGTAAAATCTGCCTGGAGCAGCTGCGGGCACCCAAGACACTGCCCTGCCT
GCATACCTACTGCCAAGACTGCCTGGCACAGCTGGCGGATGGCGGCCGCGTCCGCTGCCCCGAGTGCCGCGAG
ACAGTGCCTGTGCCGCCCGAGGGTGTGGCCTCCTTCAAGACCAACTTCTTCGTCAATGGGCTGCTGGACCTGG
TGAAGGCCCGGGCCTGTGGAGACCTGCGTGCCGGGAAGCCAGCCTGTGCCCTGTGTCCCCTGGTGGGTGGCAC
CAGCACCGGGGGGCCGGCCACGGCCCGGTGCCTGGACTGTGCCGATGACTTGTGCCAGGCCTGTGCCGACGGG
CACCGCTGCACCCGCCAGACCCACACCCACCGCGTGGTGGACCTGGTGGGCTACAGGGCCGGGTGGTATGATG
AGGAGGCCCGGGAGCGCCAAGCGGCCCAGTGTCCCCAGCACCCCGGGGAGGCACTGCGCTTCCTGTGCCAGCC
CTGCTCACAGTTGCTGTGCAGAGAGTGCCGCCTAGACCCCCACCTGGACCACCCCTGCCTGCCTCTGGCTGAA
GCTGTGCGTGCCCGGAGGCCGGGCCTGGAGGGACTGCTGGCCGGTGTGGACAATAACCTGGTGGAGCTGGAGG
CAGCGCGGAGGGTGGAGAAGGAGGCGCTAGCCCGGCTGCGGGAGCAGGCGGCCCGGGTGGGGACTCAGGTGGA
GGAGGCGGCTGAGGGCGTCCTCCGGGCCCTGCTGGCCCAGAAGCAGGAGGTGCTGGGGCAGCTACGAGCCCAC
GTGGAGGCTGCCGAAGAAGCTGCTCGGGAGAGGCTGGCGGAGCTTGAGGGCCGGGAGCAGGTGGCCAGGGCCG
CAGCCGCCTTCGCCCGCCGGGTACTCAGCCTGGGGCGAGAGGCCGAGATCCTCTCCCTGGAAGGGGCGATCGC
ACAGCGGCTCAGGCAGCTGCAGGGCTGCCCCTGGGCACCAGGCCCGGCCCCCTGCCTGCTCCCACAGCTGGAG
CTCCATCCTGGGCTCCTGGACAAGAACTGCCACCTTCTTCGGCTGTCCTTTGAGGAGCAGCAGCCCCAGAAGG
ATGGTGGGAAAGACGGAGCTGGTACCCAGGGAGGTGAGGAGAGCCAGAGCCGGAGGGAGGATGAGCCGAAGAC
TGAGAGACAGGGTGGAGTCCAGCCCCAGGCTGGAGATGGAGCCCAGACCCCAAAAGAGGAAAAAGCCCAGACA
ACCCGAGAAGAGGGAGCCCAGACCTTGGAGGAGGACAGGGCCCAGACACCCCACGAGGATGGAGGACCCCAGC
CCCACAGGGGTGGCAGACCCAACAAGAAGAAAAAGTTCAAAGGCAGGCTCAAGTCAATTTCCCGGGAGCCCAG
CCCAGCCCTGGGGCCGAATCTGGACGGCTCTGGCCTCCTCCCCAGACCCATCTTTTACTGCAGTTTCCCCACG
CGGATGCCTGGAGACAAGCGGTCCCCCCGGATCACCGGGCTCTGTCCCTTCGGTCCCCGGGAGATCCTGGTGG
CGGATGAGCAGAACCGGGCACTGAAACGCTTCTCCCTCAACGGCGACTACAAGGGCACCGTGCCGGTCCCTGA
GGGCTGCTCCCCTTGCAGCGTGGCCGCCCTGCAGAGCGCGGTGGCCTTCTCCGCTAGCGCACGGCTCTATCTC
ATCAACCCCAACGGCGAAGTGCAGTGGCGCAGGGCCCTGAGCCTCTCCCAGGCCAGCCACGCGGTGGCGGCAC
TGCCTAGCGGGGACCGCGTGGCTGTCAGCGTGGCGGGCCACGTGGAGGTGTACAATATGGAAGGCAGCCTGGC
CACCCGGTTCATTCCTGGAGGCAAGGCCAGCCGGGGCCTGCGGGCGCTGGTGTTTCTGACCACCAGCCCCCAG
GGGCATTTCGTGGGGTCGGACTGGCAGCAGAATAGTGTGGTAATCTGTGATGGGCTGGGCCAGGTGGTTGGGG
AGTACAAGGGGCCAGGCCTGCATGGCTGCCAGCCGGGCTCCGTGTCTGTGGATAAGAAGGGCTACATCTTTCT
GACCCTTCGAGAAGTCAACAAGGTGGTGATCCTGGACCCGAAGGGGTCCCTCCTTGGAGACTTCCTGACAGCC
TACCACGGCCTGGAAAAGCCCCGGGTTACCACCATGGTGGATGGCAGGTACCTGGTCGTGTCCCTCAGTAACG
GGACCATCCACATCTTTCGGGTCCGTTCTCCGGACAGTTAAAGGGGCTAGGACTAGGGTGAGAGGGAGTGGGG
AGGGAGAGGGCAGGAAGGAGGCAGAGCTGTCCGTGGGAGGTGGAGGCCGAGGACATTTTCCTGAAGGGCAGGG
GTTGGCAACTTTTCAACATGGAGTGCCAAACTGCTAACCCGTCTTCTAGTGTGTGAGAATAGGGACCAAGGTG
GTGGCGTGACCTTAACTCTCAGAAGCAGAGGAGGCAGGTGGGTGGAGGGGGATGCTGGGAGTTCACCTGCCTC
TTGCTTTTTGTGGGTGGCTGCTGCCACCACCGCCGCTGCTATATAGTTTAGGTTTCTGAGGTTTGTGAGCCTG
TCCTGCTTTGCATTCTTCAAAACCATTCCTGATAGAGTAGCCGAGGGAGAGTCTTGGGGCCAAGAAGGGCCCA
GGGTGGGTCAGGCGTGTGCACTGTGGAACAGAGCTGAGACCCCGGCCCCCCTTGGACTGGCACAGGGTCCTAC
AGAGTCAGAGGAGGCCTGAGATTTCGAATGGGCAGTGAATGGCCTCTTCTGCCAGCCACAGGCAGGATGCTCC
TTGATCACGGCATCCCCCGGCGGTAGTACAGGGTGAGCTTGCAGGAGGGTCTGCAAGTGCAGACAGCAGCTGG
GGCCAAAGAGGTGTTAGGTTCAAAGGCTGCGAAGAGAATAGGCTACTGGAAAACCAAACCCAACAGAATTCAC
GTCCAAAAATCCACATTCTCCATCGCTTCTCAGCCTTTTGGCTAAGATTAAGTATTAAAAAAAAACCAGGCTC
TGCCAGGCGCAGTGGCTCATGCCTATAATCCCGGCACTTTGGGAGTCTGTGGCAGGAGGATTGCTTGAGGCCA
GGAGTTTGAGACCAGCCTGGGCAACATAGCGAGACCCCATCTCTACAAAAGATTAAAAATTAGCTGCGTGTAG
TAGCATATGCCTTTCAACGTAGCTACTCAGGAGGCTGAGGTGGGAGGATCCCTTGAGCCGAGGAGTTTGAGTC
TGCAGTGAGCTATGATCGCACCACTGCATTCCAGACCCCATCTCGAAAAAAAAAAAAAAAAAAAAAAACAGAA
ACAACAGAAAAAGCAGGTTATCCATCACTTCTCAGCTAAGATCAAGTGTTAAAATAAATAAATAAATAAATAA
ATAAATAAATAAATAAATACCAAGTTATGAGTGTCAGAGCAGGGGAGCCCTCCACGCAGCCACGGAGCCAGTC
TGAGGGAAGAGGTACATAGGATAGAAAAATCTCTAGGCTCCAACGCTTCTGCTTCGGTGTCTTTCCATTTCCC
AGGGTCTATGTGTTGCCTTCTCTTCATTTTCCAGCAAAATTCCTTTTGAGATATGTTGGCCTTCACTCGGCTG
AACTAGGGGGTGAAGGAAGGTTGTCCTCTGTGGGCCACTATTGCTGGGGAAACAGCTAAACCCTGCAGGCCAA
GTGGAACTTGGAGGTGCCAAGAAAACCTAGAAGGTGCAATGGGGCTGCGACGGATAGAGGTGTCAAACCAGAA
GGGAGGAACCCGTCGGAGGTGGGCTGTGACCACCGGACCTTTTCATAAGTGGTGGCAGTGGTAGAATTGAGGG
GGCAAGTAGGCTTGGAGAATGTTTTTAGCCAGGGGCTGTTGAGAGATGGCAGAAGGGGGTCTTAATGTGGGGA
CTGTGGACAGTGGGGCATTCGTGGGAACCAGAGGGATTTTGGACAAGTCCAGATGGAATGTGCAGGGCATAGG
TAGAGGGAAAAGAAAATCCCTTTCTGGGAAAACCATTTCTTGTTTATTGAAACAATGCAGGACATCATCAAAG
TCCGGGTGGACACATTCCATTATCTACAAATTACAAAGGTAGGCACAGCAAAGAATAATGAAGATTATAAGAA
AACCAAGCGCCAGGCACGGTGACTCAAGCCTGTAATCCCAGCACTCTGGGAGGCTGGGGTGGGTGGATCACTC
AAGGTCAGGAGTTCGAGACCAGCCTGGCCAACATGGAGAAACCCCATCTCTACTAAAAATACAAAAATTAGCT
AGGCGTGGGAGCGGGCGTCTGTAATCCCAGCTACTTGGGAGGCTGAGGCAGGAGAATCATTTGAACCCAGGAG
GTGGAAGTTGCAGTGAGCTCAGATCGCACCATTGCACTCCAGCCTGGGTAACAAGAATGAAACTCCATCTCAA
AAAAAAAGAAAAAGAAAAGAAAGAAAACCAAGGTCCCTCTCCCTCTCCCTCTCCCCACGGTCTCCCTCTGCCT
CTCTTTCCATGGTCACGGTCTCCCTCTGATGCCGAGCCGAAGCTGGACTGTACTGCTGCCATCTCGGCTCACT
GCAACCTCCCTGCCTGATTCTCCTGCCTCAGCCTGCCGAGTGCCTGCGATTGCAGGCGCGCGCCGCCACGCCT
GACTGGTTTTCGTATTTTTTTGGTGGAGACGGGGTTTCACTGTGTTGGCCGGGCTGGTCTCCAGCTCCTAACC
GCGAGTGATCCGCCAGCCTCGGCCTCCGGAGGTGCCGGGATTGCAGACGGAGTCTCGTTCACTCGGTGCTCAA
TGTTGCCCAGGCTGGAGTGCAGTGTCGTGATCTCGGCTAGCTACAACCTCCACCTCCCAGCCGCCCGCCTTGG
CCTCCCAAAGTGCCGAGATTACAGCCTCTGCCCGGCCGCCACCCCCTCTGGGAAGTGCGGAGTGTCTCTGCCT
GGCCGCCCATTGTCTGGGATGTGAGGAGCCCCTCTGCCCGGCTGCCCAGTCTGGGAAGTGAGGAGCGCCTCTT
CCTGGCCGCCATCCCATCTAGGAAGTGAGGAGTGTCTCTGCCCGGCTGCCCATCATCTGAGATGTGGGGAGCA
CCTCAGCCCCGCCGCCCCGTCTGGGATGTGAGGAGCGCCTCTGCCCGGCCGCGACCCAGTCTGGGAGGTGAGG
AGCGTCTCTGCCCAGCCGCCCCGTCTGAGAAGTGAGGAGCCCCTCCGCCTGGCAGCCGCCCCGTCTGAGAAGT
GAGGAGCCCCTCCGCCCGGCAGCCGCCCCGTCCGGGAGGGAGGTGGGGGGTCAGCCCCCTCCCGGCCAGCCGC
GCCGTCCGGGAGGGAGGTGGGGGGTCAGCCCCCGCCCGGCCAGCCGCGCCTTCCGGGAGGGAGGTGGGGGATC
AGTCCCCGCCCGGCCAGCCGCCCCGCCCGGGAGCGAGGTGGGGGGCGCCTCCGCCCGGCCGCTGTCCCGTCCG
GGAGGTGGGGGGTCGCCTCTGCCCGGCCGCCCCGTCTGGGAGGTGAGGAGCCCCTCTGCCCGGCCACCACCCC
GTCTGGGAGGTGTACCCAACAGCTCATTGAGAACGGGCCATGATGACGATGGCGGTTTTGTGGAATAGAAAAG
GGGGAAAGGTGGGGAAAAGATAGAGAAATCAGATTGTTGCCGTGTCTGTGTAGAAAGAAGTAGACATGGGAGA
CTTCATTTTGTTCTGTACTAAGAAAAATTCTTCTGCCTTGGGATGCTGTTAATCTAAGACCTTACCCCCAACC
CCGTGCTCTCTGAAATATGTGCTGTGTCCACTCAGGGTTAAATGGATTAAGGGCGGTGCAAGATGTGCTTTGT
TAAACAGATGCTTGAAGGCAGCATGCTCCTTAAGAGTCATCACCACTCCCTAATCTCAAGTACCCAGGGACAC
AAACACTGCGGAAGGCCGCAGGGTCGTCTGCCTAGGAAAACCAGAGACCTTTGTTCACTTGTTTATCTGCTGA
CCTTCCCTCCACTATTGTCCTATGACCCTGCCAAATCCCCCTCTGCGAGAAACACCCAAGAATGATCAATAAA
AAATTAAAAAAAAAAAAAAAAAAAGAAAACCAAGAAGATGGGAAAACCATGCAGAATTGATTTGACGAGGAAA
GCCTTGGTCTCTAATACATGCAATGACTAGAACAGTATTTGGTCAAAGAACCACATAAGCTCTGTACCCTGGA
GGAAATGTATGCAGGCAAGGACTAGTAAAATAAAAAATAATAATAATTAAAAAAAAAAAAAAGAAAGGCTGGA
CGCGGTGGCTCACACCTGTAATCCCAGCACTTTGGGAGGCTGAGGTGGGTGGATCACCTGAGGTCAGGAGTTT
GAGACCAGCCTGGCCACTACGGTGAAACCCCATCTCTACTAAAAATACAAAAATTACCCGGGTGTGGTGGCGC
ATGCCTGTAATCCCAGCTACTGGGGAGTCTGAGGCAGGAGACTCGCTTGAACCTGGGAGGCAGAGGGTGCAGC
GAGCCGAGTTCACCCCATTGCACTCCAGCCTGGGTGTCACAGTGAGACTCTGTCTCAAAAAAATAATAAATAA
ATAATTAAATCCACTTGCAGTGGTCAAAAATAAATGTTAAATAAATGGGTGAGAAATCTTGCGGAGAAATAGA
CTCCTGGCACTATAAAAATGATCCTGGAGAATAATTTGGCTAGACTTTGCAAGAATGACAAAAATGATTATAA
GAATCAGTTGTCTCACTTTGGGGAATACATTCTGGGAAAATAATGTAAGAGAAAAAATATTTGCATACAGACA
TTTGAGGCTGCTCTGTATATTACAGGCAAGAACTGGAAACTGTTCGGCTGCAGGGTGACAGCTAACTAGATTG
TGGTTTATTAGGGGAATGAGCCTTTCTTTGTGTATGACAACTATGAAAACTATTGAGAAGAAATAGTTACGTG
ATACAGTCATAATCCTCAAAAACGGAGAATCAGGAATTATGATCAAATAGTGATAGTAACAAGGAAAACATCT
GTCTAAATAATTACTGAGTAGAAATAGGTTCGGAATTTGTAAATAGTCATTAATTTGGGAGATTGGGGTTTTT
TTTGTTTTGTTTTTTGTTTGTTTTTCATTATTTTGTGCTATTATTCATACGTGTACATTTCTACTTCAGTTTT
TATTATCCATTACATAACATACATGCTTGATTATTTGCTTCAAGGAAATACACCTTTATAAGTAATACTCAAT
AACTACTGGGTTTGAGAATGAAGGTGTCAGAACGAATGAGATTGTCCTATGAAAGAAGAGGCAGGAGCCAGGG
AGGAGGATCCCACCCGGCCGGGGCTCAGCCAGGAGGCAGGGCCATTGGGGCAGGGTGGCAGTCCAAGGAACCG
CTCTGGGAAGGTTTGCAAAGGTCGGGGTCCCCCCTGCCAGGTGATCGAATTATCGTGGAGTGTCTGGAAGGCG
GGGGAAGTTTTGTTGAGTTCACCAAATAACTCAGACCAACTGGAAACCAAGTGGAGTTTCTACAGGACCAACT
AGAATAGGGATCAGCTACATGGGGGGGGGGGGAGGGGGGCAGGGAACGGTGTCTGTCTTCATTGCAGCTCTGT
CTGCAGAGCCAGCACTGTGATACCTCATAGTAGGTGCTCAGTGAATGTCTGTGAAGTGAATTACTATCTAGAC
AAGGATGAGACTGGTATGACTCTCCAAAGCTTTCCCACCTCCAAAACCTCGGCTTGTGGGGAATCTCCAGTAG
GGGGCACTGTGGACACGGTTCGCATCTGCCTCCCAAGGCTGGGCTCCGCGTCAAAAAAAAAAAAAAAAAATGA
GACTGAGCCAGGTGCAGTGGTCACTCCTGTTATCTCAGCACTTTGCGTGGCCAAGGCGGGAGGATGTCTTGAG
GCCAGGAGTTCAAGACTAGGCTGGGCAACATAGCAAGACCCCATCTCTACAAAAAAATTTTTAAATTAGCCAG
AGGTGGTGGCATGCACCCGTAGTCCCAGCTGCTCGGGAGGCTGAGGCAGAAGATTGCTCGAGCCCAGGAGTTC
AAGGCTGCAGTGAGCTCTGATTGTGCCACTGCACTCCAGCCTGAGTGACAGAGCAAAACCTGGTTTCCTAAGG
GGGGGATAAAAGAGGGAGAGATGAGATCGGGGTGTCCCGCTTCTGTGCATGTGGAGGGGGTAACTGGGAACCG
TGTACTGTAGGGGATCGCACACCTTGGGGTGGAAACAGCAGCTCTCCGGAGTTGCATGGGAGCCATGGGCAGT
GGTCCTGGCTGGTGAAATGATTCTAGCCACGTGGCCCACCCAGGGGGCAAAACAATAGAAACCTTCAGAAATG
AAACGTCACCTGGCTGCAAGAAGATAGTCCCACAGGCGCCCTAGAGATGGGGATGCCAAGTGGCTTCTCGGGA
AGCTGTAAGAATCCACAGGGCATTGTAAGATGGAGGGAAATATTAAGTTTTCTTCGTAAAGAGGTGAGGGGGG
CGAGAGCAGCAAAGGACACTGGAAAATGAGAAGCATGGATGGGAAGTGTTGCATTGAGCATAATTTGGAGGTA
AAATCTCCAACACCGTAACTGAGTGGCCTGGGGACCTGATAGAAAGCCCTGCTCAGGACTAAGGCAGAGTTCC
CAGTTATATCGCAGATACAAAGAGACCAGGAACACGCTTGTAATCACAGCACTTAGGGAGGCTGAGGCGGGTG
GATGACCTGAGGTCAGGAGTTCGAGACCAGCCTAGCCAACATGGTGAAACCCCCATCTCTACTAAAAATACAA
AAATTAGCCAGGCGTGGTGGCACATGCCTGTAATCCCAGCTACTCAGGAGGCTGAGGTGGGAGAATCGCTTGA
GCCCGGGAGGCAGGGGTTGCAGCGAGCTGAGATCGTACCACTGCACTCCAGCCTGGGCAACAGAGTGAGACCC
TGTCTCAAAAAGAAAAAAAAAAAAGAGAGAAATCAGGAACATCATGAAAGATACAGCAAAATGTTTTACCCCA
GCCCACTGCACCACACCTGGGATTGTGACAATTAACTTGATAAATTGTTCCTTTTCCTGTTCGGAACTGACTA
GGTTGTGCGTGGATGGACCAGTGAAAAGAAAATCCTGTTTGGGTAGGGAAGAGGGAAGTAGAAAACCTCTTTC
GTATTCTTGGTGTCAAGAAATGGGAAGAGAATGAGGGAGCAGAGACCTGGGTTGAGTGTTGGCTCTGCACTAA
ACAGCTGTGTGATCTTGGTCTAGTGCCTTAACCTCTCTGAGCCTCAGTTTGTACATCAGTAACATTGGGATAA
TGACATCCACATGCCCACTTCACAGAGCTGCTGTGAGGACTGGATGAGAAATGTAGAGAAGTATTTCGGACAC
CATAAAGCTCAAATACAAGGTGTTGGTTAGGAAAAGTTTCAACTGACTTGTACCCACCTGATACCCCTCCATC
AGCATAAGCGTGTATGAGGATGTTAGGTACATGTGTTGTGAGTGAAAGCTCTGGAACACCCGAGTAGAGCTTT
CTGCAAATCAGCTTCTCCTTCAGAGGAGACCATTTTGTTTTGCTTTCCAGCAGTTACTTGAAGAAGCCTTATC
TGAAGTCACAACAGTGACTTCAGGAATAGAAGGGACCAATGGCCAGCTTGCTGAAATGTAATGCCATTCTTAA
AAGCCAGCTAAGAATCAGACCGTATTAGTTAAGGACCCATAAGAGTAGACATTTAGAAAAAGTGAAAAATGGC
AAGAGTGGCCCAATCTTTTGAGAGTTTAGAGAAAATATCATTTGGAAAGGGAGTAGCTCTCCTTCTATTTTAG
CCTTATTATTACGGTATCTATAAAGCAATGCCATATTCTAAATCTGCAGAAATAAAATTCTAAGGGGCGAAAG
TCCAAGGCAGAAGTCAAAAGTTTTGCAAGGAAACCGATGAGGTTGCTGACCTGAGCTGGGCAGGGTTGAACAG
GCGAGTGCCAAGATCAAGGTCTCTGATTGTCTCTCCTTGAAAGGTAAGCAGAGAACTAGGGCTATCCCATAAA
TTAAGTGGGAGATTGGAAAGGGGTGCGGACTTCTGTATTGGGGAGAAGGTCTTGGATGAACTGGGGCAGAGAA
CTGCCAGAGAGAGTACCTAGGAATACGTTGTTAGCAGGAGAAAGACCAAGTTCAGATGCAGACTTGGGAAAGT
AGGTAACTTGACCAAAGGTAGCAAAATTAGACTACCCAACTGGATTGTGAATGCAGTTTATGAAATGGATCAG
GACAGGAGCAAAAATAGATTTTCTAGAAACTGCAGACTCCTGTGATTGGAGCAGTATACAAAGTACAGCAACA
TCAGACTAGATCTCACCTGAGGTGCTGGGTGAGAAATCTTAGGGACCAGATGGAAGAGGGCCTGTGTATAAAG
ACATTAGGAAGGAGGTTTGGGAGGGCAGCCTGACTTGTCTCCAGAGAAAGTTAGAACTGGTTGACTTAGCAAC
CAAGGGACACTGGGTCAAAGACGAGGAATCAGGACGTCGGTGCCTAGTGACTGAGCGCCAGCCCAGCCCATTT
CTCTACATTGCCATGGGCTAAACCCAGTGTGCTGTAAACTCTTCAGTTCCTTTGGTTATGGATTTTGTATCTT
TTCTGTGTTTCTTAATAAAGTTCTTTTGAAAAATGGGAACCTTTCTCAGATGGGAATTTTGTATCTTTCCTCT
GTTTCTTAATAAAGTTCTTTTGAAAAATTGGAA
  (SEQ ID NO: 201)
MVSHGSSPSLLEALSSDFLACKICLEQLRAPKTLPCLHTYCQDCLAQLADGGRVRCPECRETVPVPPEGVASF
KTNFFVNGLLDLVKARACGDLRAGKPACALCPLVGGTSTGGPATARCLDCADDLCQACADGHRCTRQTHTHRV
VDLVGYRAGWYDEEARERQAAQCPQHPGEALRFLCQPCSQLLCRECRLDPHLDHPCLPLAEAVRARRPGLEGL
LAGVDNNLVELEAARRVEKEALARLREQAARVGTQVEEAAEGVLRALLAQKQEVLGQLRAHVEAAEEAARERL
AELEGREQVARAAAAFARRVLSLGREAEILSLEGAIAQRLRQLQGCPWAPGPAPCLLPQLELHPGLLDKNCHL
LRLSFEEQQPQKDGGKDGAGTQGGEESQSRREDEPKTERQGGVQPQAGDGAQTPKEEKAQTTREEGAQTLEED
RAQTPHEDGGPQPHRGGRPNKKKKFKGRLKSISREPSPALGPNLDGSGLLPRPIFYCSFPTRMPGDKRSPRIT
GLCPFGPREILVADEQNRALKRFSLNGDYKGTVPVPEGCSPCSVAALQSAVAFSASARLYLINPNGEVQWRRA
LSLSQASHAVAALPSGDRVAVSVAGHVEVYNMEGSLATRFIPGGKASRGLRALVELTTSPQGHFVGSDWQQNS
VVICDGLGQVVGEYKGPGLHGCQPGSVSVDKKGYIFLTLREVNKVVILDPKGSLLGDELTAYHGLEKPRVTTM
VDGRYLVVSLSNGTIHIFRVRSPDS*
```

An amino acid sequence for the protein encoded by the TSPEAR gene that is a regulator of T cell exhaustion/dysfunction resistance is available from the UniPROT database at accession no. Q8WU66, cDNA NM 144991, with gene location at Chr 21: 44497893-44711572.

```
  (SEQ ID NO: 202)
AGCCACTTCCCTGCAGGCTGAGCGGAGCCCTGGATGGAGCTTGGCACCCAAGCCCCTCATGTCTGCCCTGCTG
AGTCTGTGTTTTGTGCTGCCCCTGGCGGCCCCCGGCCACGGCACGCAGGGTTGGGAGCCCTGCACAGACCTGC
GCCCCCTGGACATCCTGGCGGAAGTGGTCCCTTCTGATGGCGCCACAAGCGGGATCAGGATAGTTCAGGTTCA
CGGTGCACGGGGACTCCAGCTCTCAGTAGCCGCCCCCCGCACCATGAGCTTCCCAGCATCCAGGATTTTCTCC
CAGTGTGACCTCTTCCCTGAAGAATTTTCCATCGTCGTAACTTTGAGAGTTCCCAATCTTCCACCCAAGAGGA
ACGAGTACCTGCTGACGGTGGTGGCAGAGGAGAGCGACCTGCTGCTGCTCGGCCTGCGGTTGTCACCTGCCCA
GCTGCACTTCCTGTTCCTTCGCGAGGACACGGCCGGCGCCTGGCAGACCCGAGTGTCCTTCCGCAGCCCGGCC
CTGGTGGATGGCCGCTGGCACACACTGGTCCTGGCTGTGTCCGCAGGCGTCTTCTCCCTCACCACGGACTGCG
GCCTCCCGGTGGACATAATGGCCGATGTGCCCTTCCCAGCCACCCTGTCAGTGAAAGGAGCTCGATTCTTCGT
CGGCAGCCGGAGGAGAGCCAAAGGCCTGTTCATGGGACTGGTGAGGCAACTGGTCCTGCTGCCGGGCTCAGAC
GCCACCCCAAGGCTGTGTCCCAGCAGGAACGCCCCGCTGGCGGTGCTGTCCATCCCACGGGTCCTGCAGGCTC
TCACGGGGAAGCCAGAAGATAACGAGGTGCTAAAATATCCCTATGAAACCAACATTCGAGTGACGCTGGGACC
CCAGCCACCGTGTACCGAGGTGGAAGACGCCCAGTTCTGGTTTGATGCCAGCCGGAAGGGCCTGTATCTGTGT
GTTGGCAACGAGTGGGTCTCCGTGTTAGCAGCCAAAGAAAGACTGGACTACGTGGAGGAGCATCAGAACTTGT
CCACCAACTCAGAGACCCTGGGCATTGAGGTGTTCCGCATCCCTCAGGTGGGGCTCTTTGTGGCCACAGCCAA
TCGCAAAGCCACATCCGCCGTCTACAAGTGGACCGAAGAGAAGTTCGTCTCATATCAGAACATCCCCACGCAC
CAAGCACAGGCCTGGAGGCATTTCACCATCGGGAAAAAGATCTTCCTGGCAGTGGCTAATTTTGAACCAGATG
AGAAGGGTCAGGAGTTCTCTGTCATTTACAAATGGAGCCACAGAAAGCTGAAGTTTACCCCATATCAGAGCAT
TGCCACACACAGCGCCCGAGACTGGGAGGCCTTCGAGGTGGATGGGGAGCACTTCCTGGCGGTGGCCAACCAC
CGGGAAGGCGACAACCACAACATCGACAGTGTCATCTACAAGTGGAACCCGGCAACCCGGCTCTTCGAGGCCA
ACCAGACCATCGCCACCTCCGGCGCCTACGACTGGGAGTTCTTCAGTGTGGGGCCCTACTCGTTCCTGGTGGT
GGCCAACACCTTCAACGGCACCTCCACCAAGGTGCACTCGCACCTCTACATCCGACTCCTGGGCTCCTTCCAG
CTCTTCCAGTCCTTCCCGACGTTCGGTGCTGCAGACTGGGAGGTCTTCCAGATCGGGGAGAGGATCTTCCTCG
CTGTGGCAAACAGTCACAGCTACGATGTGGAGATGCAAGTCCAGAATGATTCCTATGTCATCAACTCCGTCAT
CTACGAGCTGAACGTGACCGCGCAGGCCTTTGTCAAGTTCCAGGACATTCTCACCTGCAGTGCTCTGGACTGG
GAGTTTTTCTCGGTGGGAGAAGATTATTTCCTGGTGGTGGCCAACTCCTTCGATGGGCGTACCTTCTCGGTGA
ACAGTATTATTTACAGGTGGCAGGGCTACGAGGGCTTCGTGGCGGTGCACAGCCTCCCCACCGTCGGCTGCAG
GGACTGGGAGGCCTTCAGCACCACGGCTGGTGCCTACCTCATCTACTCCAGCGCCAAGGAGCCCCTCTCCAGG
GTCCTGCGGCTGAGGACACGCTGAGGCCGCGGCTGCCCGGCAGCAACTGGGGTGGCCAGGTGGGGGGGGACCC
CAGCACCTCCCCCAACTGACCCTGGACGTCCAGGTGGGCCCAGCCTAGGGCATCCCGGGCCTGGGCATCAGGC
AGGGTGCAGGTGGGGCCATCTGCGGCCCTGGTTCGGGCAGGGACATCCATCCACCTCTGAGTCCTTCTGAGCC
ACAGCCCCAGTGACGGTCTGGTCTTGCAGGCAGAGGCCCAGAGCCCCTGCCAGTGCTCGCCCATCTGAGTGTC
AAGCGGGGATTTCGCTGTCAAGGAACCGTTTCTATACATTTTTACCTCAAACAACAGGACCTCGGTTCATGTC
CCTGCGGCTGGCCTCTCGCCAGCCACTCCTGCAGTGTGCGTCCGGCCGCCTGAGCAGGTGTGGTGCCGTCCCG
CCATTGCCGCCACCTATTTATTGTGTTTTATCTGAACGGAAGTGTATTTATACTGCTCTTTACAAAAGCACCG
GTGCTCCTCTCTAGGCAGCGGGTGGGCACAGCCCCTCACCACGGACACGGACAGAGGACCACCTCCCCATACA
GAGAGAGGCTTCCCTCCCAGCCTCAGTTTACCCGTAAATGCACTAGCGCGAGGTCAGAAATCACAAGCCTGGG
CATGGCCTGGGTCGCACCCCCTCCCCACGGCCCCACCCACAGGGACCTCAACTTCTGGGCCCGGGATTTTTTC
CCTATTCAGCAGCATTTTAATCACATGTCCTCCAGCTGACTGGGACTCAGGCTGCGTGCCTCCGTCTGCCTGT
GTGCTGGCTGTCCTAGCTCCCTGGTAACAGCATCCTCTGCTGGCTCTCCTGTGGTCATCTTGTCTCTGCAGCT
GGGAGAGCTGCTTGTCTGTCCGTCACAGGCAGTGAGGATGAGAGGGGGTGATGGGCGCAAGGCCCCAGGGGTC
CTCCCTCTTAACCCATGTAGGTGCCAGAGAAACCACCCCCACTCCATGAGAAGAGGCTCAGACAGCAGGCTCC
GGGGCCACGCAGCCCAGACCACACCTGCCACGGTCTTCCAGGAACGGTGCTCCTGCTTCTGCAACACAAATCG
GAACAGGAGCGATCCTCAGAGAGTTTACAATGTGCTTTTTCCGCCAATCAGAACTGTGGACAAAACTTCCTAT
TTCCTACACCGCAGCTGTCTGTTCACCTTCAGCTCAGAGAGCACTCCTGACCTTTCGTAATGAAATGGAAGCC
TCCCCTTGTCTGGCAAATAATAAACTCAGTGTCTACTTTTGCCGGCTTTGGGACCTTCGTGTTTTTCCGGGGC
TCAGGTCTCACTCCAGCCATCGCTGTGGCCAGTGGAGAGCCACTCCCCTCTGGGGGCAGTCGCCGAGAGCCTC
CTTCCCCTCCGTCCAGCCAGCAAGGCTTTCCCAAGCGTCCTGGGCAGGAGGGTTGGGCCCTGCTTTCCACCAA
GTCACTCAGAAGGTCCCAATCACACATGATTGGCCTCATTGGGTCAGCACCTGTGAAAGGAAGATTAAAAACT
CGACACCCCAATTCACTCAACCAAGAAGAAAAAAGCTGAAAGCTGAGTCCTGCAGGAAGCTGCCTTTCCCTTT
GCCCTAAGCAGACAGCTGCAGATGAGAGGGTCAGCATCTCCACTAGTGGCTGCTCTGTGTGCCCCTTATCTCA
TGGAAAGTGCCAATTTACTGAGCACGAGATAAACAGGTAACTGACTGTTCCCACCTGCTCCTTTTCTCCTGCA
GCATGGAGCAATCAGTCATGTGACCACACCCTCCTCCCTCTGCCCCCTCCAGCCGGCTTTTCCCCTTGACTGA
AGCCTTCAACATCATCTTGGGAGAGAGGCACAGACCTGTCTCCTGGGTGCATCCTTAGCCTTGGCAAAATAAA
CTTCTCCATTGATCGA
  (SEQ ID NO: 203)
MSALLSLCFVLPLAAPGHGTQGWEPCTDLRPLDILAEVVPSDGATSGIRIVQVHGARGLQLSVAAPRTMSFPA
SRIFSQCDLFPEEFSIVVTLRVPNLPPKRNEYLLTVVAEESDLLLLGLRLSPAQLHFLFLREDTAGAWQTRVS
FRSPALVDGRWHTLVLAVSAGVFSLTTDCGLPVDIMADVPFPATLSVKGARFFVGSRRRAKGLEMGLVRQLVL
LPGSDATPRLCPSRNAPLAVLSIPRVLQALTGKPEDNEVLKYPYETNIRVTLGPQPPCTEVEDAQFWFDASRK
GLYLCVGNEWVSVLAAKERLDYVEEHQNLSTNSETLGIEVFRIPQVGLFVATANRKATSAVYKWTEEKFVSYQ
NIPTHQAQAWRHFTIGKKIFLAVANFEPDEKGQEFSVIYKWSHRKLKFTPYQSIATHSARDWEAFEVDGEHFL
AVANHREGDNHNIDSVIYKWNPATRLFEANQTIATSGAYDWEFFSVGPYSFLVVANTENGTSTKVHSHLYIRL
LGSFQLFQSFPTFGAADWEVFQIGERIFLAVANSHSYDVEMQVQNDSYVINSVIYELNVTAQAFVKFQDILTC
SALDWEFFSVGEDYFLVVANSFDGRTFSVNSIIYRWQGYEGFVAVHSLPTVGCRDWEAFSTTAGAYLIYSSAK
EPLSRVLRLRTR*
```

An amino acid sequence for the protein encoded by the UBAP2L gene that is a regulator of T cell exhaustion/dysfunction resistance is available from the UniPROT database at accession no. Q14157, cDNA NM_001127320, with gene location at Chr 1: 154220179-154271510.

```
  (SEQ ID NO: 204)
CCCGACTAAGTGACTTAAACTCCCACCTACTCCTGGAATAAGGAGTCAAAGCCCGGATAGGCGCAGTATTCTA
CCTTGTAAATACTGTTATTTGTATATACTGTAAATGATGACATCGGTGGGCACTAACCGAGCCCGGGGAAACT
GGGAACAACCTCAAAACCAAAACCAGACACAGCACAAGCAGCGGCCACAGGCCACTGCAGAACAAATTAGACT
TGCACAGATGATTTCGGACCATAATGATGCTGACTTTGAGGAGAAGGTGAAACAATTGATTGATATTACAGGC
AAGAACCAGGATGAATGTGTGATTGCTTTGCATGACTGCAATGGAGATGTCAACAGAGCTATCAATGTTCTTC
TGGAAGGAAACCCAGACACGCATTCCTGGGAGATGGTCGGGAAGAAGAAGGGAGTCTCAGGCCAGAAGGATGG
TGGCCAGACGGAATCCAATGAGGAAGGCAAAGAAAATCGAGACCGGGACAGAGACTATAGTCGGCGACGTGGT
GGGCCACCAAGACGGGGGAGAGGTGCCAGCCGTGGACGAGAGTTTCGAGGTCAGGAAAATGGATTGGATGGCA
CCAAGAGTGGAGGGCCTTCTGGAAGAGGAACAGAAAGAGGCAGAAGGGGCCGTGGCCGAGGCAGAGGTGGCTC
TGGTAGGCGAGGAGGAAGGTTTTCTGCTCAAGGAATGGGAACCTTTAACCCAGCTGATTATGCAGAGCCAGCC
AATACTGATGATAACTATGGCAATAGCAGCGGCAATACGTGGAACAACACTGGCCACTTTGAACCAGATGATG
GGACGAGTGCATGGAGGACTGCAACAGAGGAGTGGGGGACTGAAGATTGGAATGAAGATCTTTCTGAGACCAA
GATCTTCACTGCCTCTAATGTGTCTTCAGTGCCTCTGCCTGCGGAGAATGTGACAATCACTGCTGGTCAGAGA
ATTGACCTTGCTGTTCTGCTGGGGAAGACACCATCTACAATGGAGAATGATTCATCTAATCTGGATCCGTCTC
AGGCTCCTTCTCTGGCCCAGCCTCTGGTGTTCAGTAATTCGAAGCAGACTGCCATATCACAGCCTGCTTCAGG
GAACACATTTTCTCATCACAGTATGGTGAGCATGTTAGGGAAAGGATTTGGTGATGTCGGTGAAGCTAAAGGC
GGCAGTACTACAGGCTCCCAGTTCTTGGAGCAATTCAAGACTGCCCAAGCCCTGGCTCAGTTGGCAGCTCAGC
ATTCTCAGTCTGGAAGCACCACCACCTCCTCTTGGGACATGGGCTCGACGACACAATCCCCATCACTGGTGCA
GTATGATTTGAAGAACCCAAGTGATTCAGCAGTGCACAGCCCCTTTACAAAGCGCCAGGCTTTTACCCCATCT
TCAACCATGATGGAGGTGTTCCTTCAGGAGAAGTCACCTGCAGTGGCTACCTCCACAGCTGCACCTCCACCTC
CGTCTTCTCCTCTGCCAAGCAAATCCACATCGGCTCCACAGATGTCGCCTGGATCTTCAGACAACCAGTCCTC
TAGCCCTCAGCCGGCTCAGCAGAAACTGAAACAGCAGAAGAAAAAAGCCTCCTTGACTTCTAAGATTCCTGCT
CTGGCTGTGGAGATGCCTGGCTCAGCAGATATCTCAGGGCTAAACCTGCAGTTTGGGGCATTGCAGTTTGGGT
CAGAGCCTGTCCTTTCTGATTATGAGTCCACCCCCACCACGAGCGCCTCTTCAAGCCAGGCTCCAAGTAGCCT
GTATACCAGCACGGCCAGTGAATCATCCTCTACAATTTCATCTAACCAGAGTCAGGAGTCTGGTTATCAGAGC
GGCCCAATTCAGTCGACAACCTATACCTCCCAAAATAATGCTCAGGGCCCTCTTTATGAACAGAGATCCACAC
AGACTCGGCGGTACCCCAGCTCCATCTCTTCATCACCCCAAAAGGACCTGACTCAGGCAAAGAATGGCTTCAG
TTCTGTGCAGGCCACGCAGTTACAGACCACACAATCTGTTGAAGGTGCTACAGGCTCTGCAGTGAAATCTGAT
TCACCTTCCACTTCTAGCATCCCCCCTCTCAATGAAACGGTATCTGCAGCTTCCTTACTGACGACAACCAATC
AGCATTCATCCTCCTTGGGTGGCTTGAGCCACAGTGAGGAGATTCCAAATACTACCACCACACAACACAGCAG
CACGTTATCTACGCAGCAGAATACCCTTTCATCATCAACATCTTCTGGGCGCACTTCGACATCCACTCTTTTG
CACACAAGTGTGGAGAGTGAGGCGAATCTCCATTCTTCCTCCAGCACTTTTTCCACCACATCCAGCACAGTCT
CTGCACCTCCCCCAGTGGTCAGTGTCTCCTCCAGTCTCAATAGTGGCAGTAGCCTGGGCCTCAGCCTAGGCAG
CAACTCCACTGTCACAGCCTCGACTCGAAGCTCAGTTGCTACGACTTCAGGAAAAGCTCCTCCCAACCTCCCT
CCTGGGGTCCCGCCGTTGTTGCCTAATCCGTATATTATGGCTCCAGGGCTGTTACATGCCTACCCGCCACAAG
TATATGGTTATGATGACTTGCAGATGCTTCAGACAAGATTTCCATTGGATTACTACAGCATCCCATTTCCCAC
ACCCACTACTCCGCTGACTGGGAGGGATGGTAGCCTGGCCAGCAACCCTTATTCTGGTGACCTCACAAAGTTC
GGCCGTGGGGATGCCTCCTCCCCAGCCCCGGCCACAACCTTGGCCCAACCCCAACAGAACCAGACGCAGACTC
ACCATACCACGCAGCAGACATTCCTGAACCCGGCGCTGCCTCCTGGCTACAGTTACACCAGCCTGCCATACTA
TACAGGGGTCCCGGGCCTCCCCAGCACCTTCCAGTATGGGCCTGCTGTGTTCCCTGTGGCTCCTACCTCTTCC
AAGCAGCATGGTGTGAATGTCAGTGTGAATGCATCGGCCACCCCTTTCCAACAGCCGAGTGGATATGGGTCTC
ATGGATACAACACTGGAAGAAAATATCCACCCCCTTACAAGCATTTCTGGACGGCTGAGAGCTAATTTGGCCC
AAGGCTGGGGGCTGTGTTTTGTGTGTGTGTATAAATTTGCACTGAAGTCTTGTTTCAGAAACCAGACCACTGA
GGAGAGCCTGCTGAGCTGAGGCCATGGCCTGCGTGGCTTGGGGAAATGAGTTGGTGGATACCTTCTGGGCTTT
TGAACTTGCCCCTCCCCCATTTCCCTCTCCCCCATGTGTCTGACCCTGTCTTACCCATTTCAAGTTCAAGCGG
TGCAGCACCTTCGAAGCATCAATGCACACACCTGCTGTTGCTTTTGATTTCTGGAAGGCATGTAGTTTCAACT
TGTAACAAAAATATTTGTAGTCTTCAATAAACTGTGGTATTTCTTTAGCTAAC
  (SEQ ID NO: 205)
MMTSVGTNRARGNWEQPQNQNQTQHKQRPQATAEQIRLAQMISDHNDADFEEKVKQLIDITGKNQDECVIALH
DCNGDVNRAINVLLEGNPDTHSWEMVGKKKGVSGQKDGGQTESNEEGKENRDRDRDYSRRRGGPPRRGRGASR
GREFRGQENGLDGTKSGGPSGRGTERGRRGRGRGRGGSGRRGGRFSAQGMGTFNPADYAEPANTDDNYGNSSG
NTWNNTGHFEPDDGTSAWRTATEEWGTEDWNEDLSETKIFTASNVSSVPLPAENVTITAGQRIDLAVLLGKTP
STMENDSSNLDPSQAPSLAQPLVFSNSKQTAISQPASGNTFSHHSMVSMLGKGFGDVGEAKGGSTTGSQFLEQ
FKTAQALAQLAAQHSQSGSTTTSSWDMGSTTQSPSLVQYDLKNPSDSAVHSPFTKRQAFTPSSTMMEVFLQEK
SPAVATSTAAPPPPSSPLPSKSTSAPQMSPGSSDNQSSSPQPAQQKLKQQKKKASLTSKIPALAVEMPGSADI
SGLNLQFGALQFGSEPVLSDYESTPTTSASSSQAPSSLYTSTASESSSTISSNQSQESGYQSGPIQSTTYTSQ
NNAQGPLYEQRSTQTRRYPSSISSSPQKDLTQAKNGFSSVQATQLQTTQSVEGATGSAVKSDSPSTSSIPPLN
ETVSAASLLTTTNQHSSSLGGLSHSEEIPNTTTTQHSSTLSTQQNTLSSSTSSGRTSTSTLLHTSVESEANLH
SSSSTESTTSSTVSAPPPVVSVSSSLNSGSSLGLSLGSNSTVTASTRSSVATTSGKAPPNLPPGVPPLLPNPY
IMAPGLLHAYPPQVYGYDDLQMLQTRFPLDYYSIPFPTPTTPLTGRDGSLASNPYSGDLTKFGRGDASSPAPA
TTLAQPQQNQTQTHHTTQQTFLNPALPPGYSYTSLPYYTGVPGLPSTFQYGPAVFPVAPTSSKQHGVNVSVNA
SATPFQQPSGYGSHGYNTGRKYPPPYKHFWTAES*
```

An amino acid sequence for the protein encoded by the UBE2F gene that is a regulator of T cell exhaustion/dysfunction resistance is available from the UniPROT database at accession no. Q969M7, cDNA NM_001278305, with gene location at Chr 2: 237966827-238042782.

```
  (SEQ ID NO: 206)
GTTACCGAAATGCTGCCTTAGAGCGTAATTTGCAGGAAAGAGGAAGTTTTGTTTTAAACCAGATCTTCAAGAG
TTGAAGCATATTGAAGAAGGAAAAGAATTCTGAAGCAACTGCAGGGTAAAGGCAGCAGTAATGCTAACGCTAG
CAAGTAAACTGAAGCGTGACGATGGTCTCAAAGGGTCCCGGACGGCAGCCACAGCGTCCGACTCGACTCGGAG
GGTTTCTGTGAGAGACAAATTGCTTGTTAAAGAGGTTGCAGAACTTGAAGCTAATTTACCTTGTACATGTAAA
GTGCATTTTCCTGATCCAAACAAGCTTCATTGTTTTCAGCTAACAGTAACCCCAGATGAGGGTTACTACCAGG
GTGGAAAATTTCAGTTTGAAACTGAAGTTCCCGATGCGTACAACATGGTGCCTCCCAAAGTGAAATGCCTGAC
CAAGATCTGGCACCCCAACATCACAGAGACAGGGGAAATATGTCTGAGTTTATTGAGAGAACATTCAATTGAT
GGCACTGGCTGGGCTCCCACAAGAACATTAAAGGATGTCGTTTGGGGATTAAACTCTTTGTTTACTGATCTTT
TGAATTTTGATGATCCACTGAATATTGAAGCTGCAGAACATCATTTGCGGGACAAGGAGGACTTCCGGAATAA
AGTGGATGACTACATCAAACGTTATGCCAGATGATAAAAGGGGACGATTGCAGGCCCATGGACTGTGTTACAG
TTTGTCTCTAACATGAAACAGCAAGAGGTAGCCCCCTCTCCCGTCCTCATGCTCCCTCTCAGTCCCCTGGATT
GCCCCAGTCCTGTGACCATGTTGCCCTGAAGAAGACCATCTTCATGACTGCTCATTGTAGATGGAGAATTCAA
CATAAATACAGCAAGAAAATGTGTTTGGGCTTCTGAAGAGTTGTCTGCTTACCTTAACATGTTTACTTTTTTG
AACTTGTACTGTATAGGCTGTTGGTGAAATTCTTAAGAAGTTGTAATGAACTCAAAATTGAGGCCAGAGCTTG
CTTTCCCTTTTCCCAAACAAAATTGGTTTTCTGCACAAGCGATGCTAATGATGTGTTCAGTGTAACTCGCAGA
TTGGCAATAAGATACCCGCTACAAACTGTGATTGGATGCAAAATCTCTTAGCTTCTTTCACGAATGTTGGCCC
TGCCTAGATGTTGTGAAGCCTCCCAGAATGCATAGAGTCATTCACTGTAGATCTCTTATTGAAATGCGTATTT
TATTTAATGTAAGTATATTTTGGAACAGATTTGTAATTTGTACAATTCAATGCTTTAATTATTTTTTCTATTC
TCATTTAGTTTGTATTTTCATTGTATAGAGCAGACAGAAAGATGTTGGGTCAAGCAACTATTGAAGAGAAATA
CAAAGAAAATATGAAAGGCACATTATTCATTTTGTCCAAATGCAATGAGAATCTCACTCTTAAAAATCAGCTC
TTGCTTTCGGGTCCGGATGTGGTGAGCACATTTTGGAGCCCTTTGAAGCTAGATTTGGATGATCAAAACAAAA
AGGCAGGGAGCCCATTCTAACATGCTGCCCAGAGGAAACTGGCTGGAGCCTGGACCAGCTGGGGTTGATGCTT
TTGCAGTGGTCATGTGATTGTGACCTGGTAGCTACTTATCAGAGAGCCAGACCCTGCTGTCCTGGGAGACAGG
AGCGATGCCTCAGGAATCAGCCCAATGTCTGATGTCACTGAGACTGTACCTGTGGCCTTCTTCTGAGTTTGCT
ATGGCTCCAGGCCCTGCCGGTGGGGTGAGCCTCCTAGGCCTTGGAGGACCAGGAGTCAACAGTGGCATATGCC
ATCCTCGGCCAGGTTAATATACTGCAGAGGAAAAGCCCTGAAGAGAGGCAAGTGGATTTACTCCAGCATGTAG
ACATTTGAACCAGTGAAATCAAACACAAAATAAATGTCTGTCTAGTTTCATTTGCTGCCTGCCTAACACTCTC
AAACTTAACTGCCAGCTTCCTTAGACCTCTGGATTCCCCCATCTGCCATCTACTGGGTTTAGTTTGTTTTTCT
TAATTTATAGAATCTCTGATTGTGAGATCAGTCTGTCACGGAGACCCAGCAGGTGAGGAATGTAGGCCTTGCT
TCCTCTTTGCACCCATTA
  (SEQ ID NO: 207)
MLTLASKLKRDDGLKGSRTAATASDSTRRVSVRDKLLVKEVAELEANLPCTCKVHFPDPNKLHCFQLTVTPDE
GYYQGGKFQFETEVPDAYNMVPPKVKCLTKIWHPNITETGEICLSLLREHSIDGTGWAPTRTLKDVVWGLNSL
FTDLLNEDDPLNIEAAEHHLRDKEDERNKVDDYIKRYAR*
```

An amino acid sequence for the protein encoded by the UBE2L3 gene that is a regulator of T cell exhaustion/dysfunction resistance is available from the UniPROT database at accession no. P68036, cDNA NM_001256355, with gene location at Chr 22: 21549447-21624034.

```
  (SEQ ID NO: 208)
AGTATGCAGGTCGCTGCAGGGACCCGAGGCGACACGCGCCTGCAGGAGGTGGCACTCCTGCCGCAGCTCTTCG
ATCTGCTTGTCCTTGGCCAGCGGCGCGCTCGCCTGCTCCGACAGGTCCCGAGCGCGCTGGCGGGCAAAGACTT
GGCACAGCTCCAGGCCGGCGCAACTCTCGCCGGGTATAGGCGCGCTCACGGCCCGGAGGAGCTTGAAGAAATC
CGCAAATGTGGGATGAAAAACTTCCGTAACATCCAGGTTGATGAAGCTAATTTATTGACTTGGCAAGGGCTTA
TTGTTCCTGACAACCCTCCATATGATAAGGGAGCCTTCAGAATCGAAATCAACTTTCCAGCAGAGTACCCATT
CAAACCACCGAAGATCACATTTAAAACAAAGATCTATCACCCAAACATCGACGAAAAGGGGCAGGTCTGTCTG
CCAGTAATTAGTGCCGAAAACTGGAAGCCAGCAACCAAAACCGACCAAGTAATCCAGTCCCTCATAGCACTGG
TGAATGACCCCCAGCCTGAGCACCCGCTTCGGGCTGACCTAGCTGAAGAATACTCTAAGGACCGTAAAAAATT
CTGTAAGAATGCTGAAGAGTTTACAAAGAAATATGGGGAAAAGCGACCTGTGGACTAAAATCTGCCACGATTG
GTTCCAGCAAGTGTGAGCAGAGACCCCGTGCAGTGCATTCAGACACCCCGCAAAGCAGGACTCTGTGGAAATT
GACACGTGCCACCGCCTGGCGTTCGCTTGTGGCAGTTACTAACTTTCTACAGTTTTCTTAATCAAAAGTGGTC
TAGGTAACCTGTAAAGAAAGGATTAAAAATTTAAGATGTTCTAGTTCTGCTCTCTTTGTTTTAAAAATCACTG
CTTCAATCTACTTCAAAAGAATGGTGTTTCTTTTCTTGTCCAATTTTATCCAAAATCTTCAAGTTACATTTAA
CCCATAAGGTTTAAAAAAAAGGAAAAAAAACGGTTGTGGTTCCCTTTCTTCCCTACCCTTGCCACTCCCACTT
TCTGGCACCGAGTTTATTTTTCACTTACTTACTTCCCCAGACCCCGGGCTCGCCTCCACAAAGGAGAAGAGAC
TGCCCTGGCGGTCCTGGTGGCTTTTCTTAGCATGTGTGGCACTGTTGCCCAGTGTGGGAGTTGGTTTAAATTC
TCCTGACTCCAGTTTATAACATCCTTTTAAAAAATTTAAAAACAAACAGCCACACCCCTCCTCCAGTCCTTCT
CCTCAGTTCTTGTGTGAAACTCCAGCTGATGTTACCACAGTAACATCAGTTAATTGGGCAAGCCCTGATGTCA
GTGTGTGTAACTGACCTCTGGCCTGGCCTGCACAGAGAAGCCCTATAATCACAGGTCTGTGGTGGCCCCGAAA
TGGGGGGCCTGCTAGTCAGGAGGATGCTGTGCACACTGTGTGTGATGAATCTCGCCAGAAAGGCTCCTGAGGT
CCCAGGTTGGCACTTCTCCCTGCAGCCATTGTAGAAGATCTGCTGGTCCTTGCAGGCAAAGCTACAGCCAGAA
TGTCCGTTTGAAACTCCTAGCTCATCTGTCACCGAGCTTCATCCGAATGTGCCACGGAGCTTGCTCTCCACTT
CCTCCGTGCAGTGGCCCTGCCACAGCCCTCCCTCGGCACACTTTGACCCTTTGTAGGATTGGAATTAGCAGGA
CTCGGCTATTTAAAGCACCAGTCTGGGGTCGCCTGGGCCCCTGCTGACCCCCTCCTCCAGAGCAGCCAGCCCA
GCCCGGGAACAAGACGGACTTCCTCTCCCTTCGGACTCACAGCCTTTGCAGAGTCAAGCTCCACTTGAAGCTC
ACTCAGTAATATCCTTTCAATGTGTTTTATATTGTTTTGACTGCCTTTTTTTGTAGAAATAAAAATTGACCTT
AGAATTTATCGTCAGATAAACTTGTAAAGATTTGAATATTAATGTCTTTTCAAGGCAAATGGGATTGTCCCCG
CACTAGTAGAGAATCCATGTCGCTCTGACACCCCAAGGAAGCCGACGATCCAAATGCCGTGTGTCACCAACCC
CGCTTCTGCCACTGGCGGCTTCCCTTCTTGGCTCTTGGGGGGGACTAGATCCTGTGGAGAAGATGACTTAAAC
TTTGCTTTTTGTTTTAATTTTAATTCTATAACTTGAGATCTTTCCGGGGCCTACAGGCGTGTAAGACAGCTTG
GTCTGGTCTGTGCAGAAGTGGGGAGTGATGGGCAGGTTCGGCAGCCTAACATTGTTCAGGCGCATGGCCCCTG
CGGTGTGTACACGAACTCGGCTTCTTTTGTCCTAGGTACGCCAAGGGCAGGTTTCTGGAGACTCCCTTGTGCC
CGGGATGGCAAGGGCACCGGGCTGGCGTTTCCACATCTGTCTTCATTAGCAGAAAAGTGATGATGGATTTTAT
TTCACTCACACTCCAGTTTGTAATAAAATGCCAAATTCTGTCAGCTATCCAAACAAGCCACCATTTGTTCTTG
TTGCTTCTCTGGATCCAGAAATGTTGCCATTCTTGGAAACTGTCCCATTGCTTCGTATTTCTGCCAACGTAGC
TCTGCCTGCCTGTCAACCCCTCACTGCACTCTGCTCATCACGGGAGGATACCTGTGTGCCGGCAGCCCCTCAG
GGACTCTCAGCCCTGGCACTGGCACCCCAGGGTTGGCCCCGTCAGCAGAGGCTTGGCTTTCGAGCCAGTGGGT
GTCTCTCCTTTGGGCCTGGGCGGCTTGCTCCTGCCAGCCATGCCTTCAGGGTAGGCTCTGAGCAAGCTGGCGA
ACAGCCCTGGCTGCTCCAAAACCAAAAAGCTGGGTCCTCTGGAGGAGGGGCGAGCTGTGGAGCAGCCACCCAC
TGCTGCCCCAAGCTCACTCAGGAATTCACACCCGCCTGGTTTCTTGAAGTGTGCTGGGTCCTTCCCTCTGCTC
CCTACTCCCCACCACGGCAGAGAATAGGCTTTCTAAGATGCTGCGATCCCGTTCTGCTGCCCGTAATAAAAAT
GCTCTCAGACACTG
  (SEQ ID NO: 209)
MQVAAGTRGDTRLQEVALLPQLFDLLVLGQRRARLLRQVPSALAGKDLAQLQAGATLAGYRRAHGPEELEEIR
KCGMKNFRNIQVDEANLLTWQGLIVPDNPPYDKGAFRIEINFPAEYPFKPPKITFKTKIYHPNIDEKGQVCLP
VISAENWKPATKTDQVIQSLIALVNDPQPEHPLRADLAEEYSKDRKKFCKNAEEFTKKYGEKRPVD*
```

An amino acid sequence for the protein encoded by the WDFY1 gene that is a regulator of T cell exhaustion/dysfunction resistance is available from the UniPROT database at accession no. Q8IWB7, cDNA NM_020830, with gene location at Chr 2: 223855716-223945357.

(SEQ ID NO: 210)
GTCAGCTGATGGGCTGCCTGCCGAGGAGGCCGCAGCAGTCGCCGCGCGAACATGGCGGCCGAAATCCACTCCA
GGCCGCAGAGCAGCCGCCCGGTGCTGCTGAGCAAGATCGAGGGGCACCAGGACGCCGTCACGGCCGCGCTGCT
CATCCCCAAGGAGGACGGCGTGATCACGGCCAGCGAGGACAGAACCATCCGGGTATGGCTGAAAAGAGACAGT
GGTCAATACTGGCCCAGCATTTACCACACAATGGCCTCTCCTTGCTCTGCTATGGCTTACCATCATGACAGCA
GACGGATATTTGTGGGCCAGGATAATGGAGCTGTAATGGAATTTCACGTTTCTGAAGATTTTAATAAAATGAA
CTTTATCAAGACCTACCCAGCTCATCAGAACCGGGTGTCTGCGATTATCTTCAGCTTGGCCACAGAGTGGGTG
ATCAGTACCGGCCACGACAAGTGTGTGAGCTGGATGTGCACGCGGAGCGGGAACATGCTCGGGAGGCACTTCT
TCACGTCCTGGGCTTCGTGTCTGCAATATGACTTTGACACTCAGTATGCTTTCGTTGGTGATTATTCTGGGCA
GATCACCCTGCTGAAGCTTGAACAGAACACGTGTTCAGTCATCACAACCCTCAAAGGACATGAAGGTAGTGTC
GCCTGCCTCTGGTGGGACCCTATTCAGCGGTTACTCTTCTCAGGAGCATCTGACAACAGCATCATCATGTGGG
ACATCGGAGGAAGGAAAGGCCGGACGCTGTTACTTCAGGGCCATCATGACAAGGTGCAGTCGCTGTGCTACCT
TCAGCTCACCAGGCAGCTCGTCTCCTGTTCCTCGGACGGCGGAATTGCAGTGTGGAACATGGATGTTAGCAGA
GAAGAGGCTCCTCAGTGGTTGGAAAGTGATTCTTGTCAGAAATGTGAGCAGCCATTTTTCTGGAACATAAAGC
AGATGTGGGACACCAAGACGCTGGGGCTAAGACAACATCACTGCAGGAAATGCGGGCAGGCTGTCTGCGGGAA
GTGCAGCAGCAAGCGCTCAAGTTACCCAGTCATGGGCTTCGAGTTCCAAGTCCGGGTTTGTGATTCTTGTTAC
GACTCCATCAAAGATGAAGATCGGACTTCTCTAGCGACCTTTCATGAAGGAAAACATAACATTTCCCACATGT
CCATGGACATTGCCAGGGGACTGATGGTGACCTGTGGGACCGACCGCATTGTAAAGATCTGGGACATGACACC
TGTGGTGGGCTGCAGTCTGGCGACTGGGTTTTCTCCGCACTGATCTGAGAGCTGGGCGGCGTCCACACCTAAG
AACAGCAGCTCCACCAAATGAAGTCCCTCTCACGCAGCTCCACAGCGCTGTCTCGTGAATGGACAGTAGCCAC
TTACAAACAAATCAACATTTTTAAAAAGAAAATGTAAAGGTGTGTTTTGGGGCATTTGTGGAACTTACCCATG
GGGACTAATATGGAAAAGGTCTGTCCATAGTGGTTCCCTGAAGACTGGAATTACTTCAGCAAAACTTCCCCAT
GAACAGCTAATGTGTAGTGAAAGAATGAGCTAGCAAATGAGTTTTAGCGGGGACAAAAAATCAAACAAAAAAG
TGAATGCTTAGAACCTTCTCAAAGCAGTCACAAGTACAGACACTTCACTTAGCCTAGGGGGCCTTCCAGGGTT
CTTGTGGCTGTTGTCAGAGCAGGAGCTGGGGGAGGGAAGACTTGTTCTCTCTTTCTTGAGGGGTGGCATTAGG
AACTTACGAAACCAGAGACCTTTCCCTATGACTTGGCAGTATGTGAATATCCTCTACACTTAGTTATTGATAA
ACTTCTTAAAGAGATCTGTTATTTTCAGGTAGTGCCATAATCTGCACTTAGCATTGGCTTGCTTCAGTTGTTT
CTCTTCCCAGCCAGTATGCCACAGGTGAACTTTCGGGGTTGTCATTAAGTAAGTTGTGAAATTTCTGTAATAA
CAAAGGCAGTCCGCATTCTTCCCTTTCCCCCAAATTCCTAGGGCAAAACTTTTTTATGGTGCTGTTAACATGG
GAGTCACACAAGCCGCCTGACTTTTTCTCATTGCCATTAGTAATGACTGATGGAAAACCCAGCCACCACTGTG
ATGCGAAATGATCAGTCTGTTGCCTGAAACAGCCCAGTCCTCTTAACTGAAACAGCATTCTACTTCTTGTTCC
AAGATGAGCCTCTGCAATATTCTGGCAATTTAATATACCCCCTACAAAAGCACTCCACAGCTTTTACACTATT
TTGACTTTGAGTTATAACTAGTATTATTCATGTTTTCATAAAAAGAAGTTAGTGACCCAGAGCTATAATCATC
CATCAAGTCTTCCACAATAATTCCCAACTCATAAATTGCTTTCCTAACAACTAGCAAAAGCTATTGTTCATAA
TGGCATTTCTAAAGCTTTTGGGCACTGTGTACCAGGATGAGGAAGAGAAGAAATGAGGAGCCTGTCTTTTAAT
ATTCCAGTATTTGTGTGTTTGATTTTTTTTGACAACAGTACATATATCTATTTCTCTAGGGATATGGAAGTAA
GTGGAGAAGGGCCTACCTTTTTAAAGGACAAATATAAAAATAGCAACAGTATCTTTGCTAATCTTACTAATAG
ATATTGATTAAAAAAAAAAAACCTCAGTACTGCATCACTGTGTTGGGATCATACCAGGACAATAGGGTCATTC
CATATGATAAAACTAAAGGACTAAATTTGTTTTATAATGATGTCTTAGAGGGACTGAAAGGTTTAAGGAGGCG
ATCAAACTAAAATGTCTTAATGGCTGTGTGACACACGTAAGGAAAGGAAAGGGGGTCACGCACATCATGTACT
GGAATGATCTGCATTAAACATAGACTTGTCTTCAGAAATAAGACTGAAGGGTTTTGTTGTTCCTTAGAGTTTT
CGTGTTACATCACCTAAAGAGATTTCTTTTAAAAACTTTCTAGACTCTTTGCAAAATGTATATTACTAACATA
GTTTGGAAGAAAAATTGAGTAGTGGTAAGTTTTGTTCAAGCACAATGTTGAATGTTAAGCTTCCTGTATTAAT
TTTAGTTCAGTTAATGGTTCAGCCCATACAAAGGTGCTATCCTAGGGATTTTATGAATTCCTGAAAGGAAAAT
AGAATATGATCAATTCCTTGCCCTGTGGAAGAGTGCAGAACTGTGGTTTTGTTTTCCTTTGACTTCTGTAAAA
TGTGACCGTTTGACATCTGTGGTAGATTGAACGGAATATCACAGCTGCTGAGTTTACTCCATAGCTTTCAAAC
CTTTTTTATTTTAAGAAATTCTTGAAAAACCCTATGTTCCATGGGAACATAAAGTTATTATAGTGCCTCCTAA
GGGGTTAATATAAATCAAGGAGGAAGTTTATATTTAAGGAAGAATTGGAGTGACGTATCTTAGAAAAGGAAAG
GCTGATGTTTCCATATAGCTTGCTCTCCACCAGGCCTATCATTTTTCTTTTTAGAGACAAACGTGACAGGCTA
GTCTTGTCTCTCTCATATGCTAGGTAGCAAATGGGGTGATATTTTTATAGAAGTGGGCAAAAATTATTTTCTC
AATTTTACTGAGTAGGCACAGAAGAAAAGTACAGATAGGCTGATGGTTATTGCCTTATTTTGACTGCATTTCT
CTTAAATGGATCATTTAAATTAGTTCTTCAAGTAACAGTTTACTGGTTGTTCCATTCCTGAATATGCAGGCTA
ATTTGTACAGATAGGGATTAAGGAATACAGACTATTAGAGAAGATCCTTATATTTACATCTAGTATATATGTG
GTAAGGAAATGCCGATTCTTCATTATAAACAAGTTTTAAAATTGTTCTTTCTTAGTTCAAATGATAGCAATAC
CCCTATAGCATTAGGTAGAAACAAATTATTCATTACATCGTAAATCTCTTTACTATGTCCTAGCTCTGTCCTG
CTACCTAAAGGATATAAAGAAATACTATTGCTCTAGAATGTATTACTTTGTTCTCCCATGAAAGAATTCAGTT
TGTTAGTACCTATATTTTTAAACTGGTGAAACTGACCCAAATATGTAATAAATACCATAGTAGCTCAGACCCA
AGGAGATATTTTTCTAAAATCAGTTTTCGTTAAAGTACTTCTACTTCCGTTATTGGATATGGTATCTCCTAAA
GTGTAAAAAAAATCTGTTACTATATAGTGATAAACCATCTGCTCATCGTAAGTGTAAGGCTTAACAAATAAGT
AATACATGCTATATTTATTCAAGTGTCTATTGCTTAATTGTTAATTGTGAGCAGATTTATTGAATGCCTATTC
TATTTTCTGCAGTTTACAATACAATAACTCTTTGAGTAAGTTGAAGTTTAATTGTGCAACAAATTTGTATTAG
AGTACAATTTAAAGTGTTTTTCTCTATAGCCTTTTTTGACTGGGGAAGCAAGGGGTAATGTTAATTAGTACAC
TTTGTTCTTGTACTAGCTATGTTTCTATAAGATATGGTGCCCTGTGTATCCCAGAGATGCTAGAAAACTGTTC
TTTGCTCCTATTTGTGGGTTCTGTTTTTGTGGGTTTTTTTTTTTGAGAAAATGTACACAATAAAACATTCCTT
GCTTGTTA
(SEQ ID NO: 211)
MAAEIHSRPQSSRPVLLSKIEGHQDAVTAALLIPKEDGVITASEDRTIRVWLKRDSGQYWPSIYHTMASPCSA
MAYHHDSRRIFVGQDNGAVMEFHVSEDENKMNFIKTYPAHQNRVSAIIFSLATEWVISTGHDKCVSWMCTRSG
NMLGRHFFTSWASCLQYDFDTQYAFVGDYSGQITLLKLEQNTCSVITTLKGHEGSVACLWWDPIQRLLESGAS
DNSIIMWDIGGRKGRTLLLQGHHDKVQSLCYLQLTRQLVSCSSDGGIAVWNMDVSREEAPQWLESDSCQKCEQ
PFFWNIKQMWDTKTLGLRQHHCRKCGQAVCGKCSSKRSSYPVMGFEFQVRVCDSCYDSIKDEDRTSLATFHEG
KHNISHMSMDIARGLMVTCGTDRIVKIWDMTPVVGCSLATGFSPH*

An amino acid sequence for the protein encoded by the ZBTB21 gene that is a regulator of T cell exhaustion/dysfunction resistance is available from the UniPROT database at accession no. Q9ULJ3, cDNA NM_001098402, with gene location at Chr 21: 41986831-42010387.

(SEQ ID NO: 212)
GACGTCAAATTCAAGATGGATGGAATCACAGCGGCAGCGGCGGCTGCGGCGCGCGCGAGCCGAGTGTGAGCGG
AAAGGGGCCCGGCGTCTGCCTCGAGACTGAAGACCGAAGACATGAAGTAGAAACAATCCAGGAAGGCTTTGGG
AAGCTATTTGTGTGCGAGGATCAAAGTGATAAACTCAAGCCATGGAGGGATTACTGCATTACATCAACCCTGC
ACACGCCATTTCTCTCCTAAGTGCCCTGAATGAGGAGCGTCTCAAAGGACAGCTGTGTGATGTGCTGCTGATT
GTTGGAGACCAAAAGTTCCGAGCTCATAAAAACGTCTTGGCTGCCAGCAGCGAATACTTTCAGAGTTTATTCA
CAAATAAGGAAAATGAGTCACAAACTGTATTTCAGCTTGACTTCTGTGAGCCAGATGCTTTTGATAATGTTTT
AAACTACATTTATTCTTCCTCTCTATTTGTTGAGAAGAGCAGCCTTGCTGCTGTGCAAGAACTTGGCTATAGT
CTTGGGATTTCCTTTCTGACTAACATCGTTTCTAAAACACCTCAAGCCCCCTTTCCAACGTGTCCTAATAGAA
AAAAAGTGTTTGTAGAAGATGATGAAAACAGTTCTCAAAAGAGAAGTGTCATTGTTTGTCAAAGTAGAAACGA
AGCGCAAGGAAAAACTGTTAGTCAAAATCAACCCGATGTAAGCCATACTTCCCGGCCCTCTCCTAGCATTGCA
GTCAAGGCCAATACCAATAAGCCACATGTCCCAAAACCAATAGAACCACTTCATAATTTGTCATTAACTGAAA
AGAGTTGGCCGAAAGATAGTTCTGTGGTATATGCAAAGTCTCTTGAGCATTCTGGATCTTTGGATGATCCTAA
TAGAATCAGTTTGGTGAAAAGAAATGCAGTGTTGCCTTCAAAGCCTCTGCAAGACAGAGAAGCTATGGATGAT
AAACCAGGTGTGAGTGGTCAGCTTCCAAAAGGAAAAGCTCTAGAGCTGGCTTTGAAGAGACCACGGCCACCTG
TTTTGTCTGTTTGTAGCTCATCAGAGACTCCCTATCTATTAAAAGAAACTAACAAAGGAAATGGTCAAGGTGA
AGATAGAAACTTGTTGTACTATTCAAAGTTAGGCTTAGTGATCCCATCCAGTGGATCTGGTTCTGGAAACCAA
AGCATTGACAGGAGTGGCCCACTTGTTAAGAGTCTCCTCAGACGGTCATTGTCGATGGATAGCCAGGTTCCTG
TCTATTCACCTTCCATAGATTTGAAATCTTCCCAGGGATCATCTTCGGTGTCCAGTGATGCACCAGGGAATGT
GTTGTGTGCTTTATCTCAGAAGTCATCTTTAAAAGATTGTAGTGAAAAAACAGCCCTAGATGACAGGCCTCAA
GTGCTACAACCGCATCGCCTCAGGTCCTTTAGTGCTTCTCAGTCAACAGACAGGGAGGGAGCTTCCCCTGTGA
CTGAGGTGCGCATAAAGACTGAGCCCAGCAGCCCGCTGTCGGACCCCTCGGACATCATCCGCGTCACTGTGGG
AGATGCGGCAACAACAGCAGCTGCCTCATCTTCGTCGGTCACAAGAGACCTGTCTCTGAAAACAGAAGATGAC
CAAAAAGACATGAGCAGACTCCCAGCAAAAAGGAGGTTCCAAGCGGACCGAAGATTGCCGTTTAAGAAGTTAA
AGGTGAATGAGCACGGGTCTCCTGTGTCAGAAGATAATTTTGAGGAAGGCTCAAGCCCTACTCTCCTTGATGC
AGATTTTCCAGATTCTGATTTGAATAAAGACGAATTTGGTGAGTTGGAGGGGACGAGACCAAACAAAAAATTT
AAATGCAAACATTGCCTTAAGATCTTTAGATCAACAGCAGGTCTTCACCGTCATGTTAACATGTACCATAACC
CAGAAAAGCCCTACGCTTGTGACATCTGTCACAAGAGGTTTCACACCAACTTCAAAGTGTGGACACACTGTCA
GACCCAACACGGCATAGTGAAGAACCCATCACCAGCCTCTAGTTCACATGCTGTTTTGGATGAAAAATTCCAA
AGAAAGCTGATTGACATAGTGAGAGAGAGAGAAATTAAGAAGGCCCTGATCATTAAGTTAAGGCGCGGCAAGC
CTGGTTTTCAGGGACAGAGTAGCTCCCAAGCACAGCAAGTCATCAAGAGGAACTTGAGATCTCGAGCCAAAGG
AGCTTACATTTGTACTTACTGCGGAAAAGCGTACCGCTTTCTCTCTCAATTTAAGCAGCATATAAAAATGCAT
CCAGGAGAAAAACCCCTTGGAGTAAATAAAGTTGCTAAACCAAAAGAGCATGCTCCTCTTGCAAGTCCAGTAG
AAAACAAGGAGGTTTACCAGTGCCGCCTCTGTAATGCTAAGCTCTCTTCTCTCCTAGAGCAAGGAAGCCACGA
GCGGCTGTGCCGGAACGCGGCCGTCTGCCCTTACTGCAGCCTCAGGTTTTTCTCGCCCGAGCTGAAGCAAGAA
CACGAGAGCAAGTGTGAGTATAAGAAGCTGACCTGCCTCGAGTGCATGCGCACCTTCAAGTCCTCTTTCAGCA
TCTGGCGGCACCAGGTTGAAGTCCATAATCAGAACAACATGGCACCCACCGAAAACTTTTCTTTGCCCGTTTT
GGACCACAATGGTGATGTGACTGGTTCTTCAAGGCCCCAATCCCAGCCTGAGCCCAACAAAGTAAACCACATC
GTCACCACAAAAGACGACAACGTGTTCAGTGATTCTTCAGAACAAGTTAACTTCGACTCGGAAGATTCCTCTT
GTCTTCCCGAAGACCTTAGTCTTTCCAAGCAACTGAAAATCCAAGTCAAAGAGGAGCCTGTGGAGGAGGCTGA
AGAAGAGGCACCCGAGGCCAGCACAGCCCCCAAAGAAGCGGGTCCTAGCAAAGAAGCCAGCCTGTGGCCCTGC
GAGAAGTGTGGGAAGATGTTCACGGTGCATAAGCAGCTGGAGCGTCACCAGGAGCTTCTGTGCTCTGTGAAAC
CATTTATTTGTCACGTGTGCAACAAAGCTTTTCGCACTAATTTTCGACTCTGGAGTCACTTCCAATCGCACAT
GTCTCAGGCTTCAGAGGAATCGGCACATAAGGAATCTGAGGTGTGCCCTGTTCCCACAAACTCTCCCTCTCCA
CCACCTCTGCCACCGCCACCACCACTGCCCAAGATCCAGCCTCTGGAGCCTGACAGCCCCACAGGCCTGTCCG
AAAACCCAACTCCAGCCACAGAAAAACTGTTTGTGCCCCAAGAATCAGACACCCTTTTTTACCATGCCCCACC
CCTTTCAGCAATCACATTTAAAAGACAGTTTATGTGTAAACTTTGCCACAGGACATTCAAGACTGCATTTAGT
CTTTGGAGTCACGAACAAACACACAATTGAAAGACCAACACTTTTTACCTATGGGAGGCAGTCCCCAGATTTC
AACCTGAATTGTGAAATGTGTCATAAGAAACAAAATATTTTTTAAACAAGAATAATAAAGGTTGGAGATTGTT
ACGCTTGAAATTAAGTTGGAGGCAAATTGATAATTACTTAGTAATGTCCTTAACTTTAGAAATACAGCTTTTA
AAATATTGTGGTTCTGGACCTTACAAGAACAAGATGGTTTGATTTCATTAATGTTGAAATTGGATTAGTCTTG
ATTGTGTGCATGGTTCCTCATTCCATGGTGTCAGTATAATCATTTAATTGAGGTTTTTGGTTTTTTATTGATT
AGTGGAATACTGTCGAAGTATTCCTTCTTTTAATTATTTTAGACACTTGAGTTCTAGTTAAATCTTAACCATG
TTCTTAAGTCCAAAATAGGAAGAAGTGGAGTAGTTTGCAGTATTGATTTATATCTTAGACTACTTTAGCAATA
AAAGATAAATCTCAACTTTAATAAGTTATCCTGACACTTGATAAAAATAAATATTTGGTATTTTGTGGTCATG
TAAAACTTCTTTTTGTATGAAAATTGTGAGAAATTTAAATCAGCAATAGTAACACTTAGATTTTTATAAACTA
ACAAAAACTTGCTCTCAGTCAATATAATAAATGATAGTTAAACTCTAAGGAAGACCTGGGAAATGAAAACAAT
TTATGGAAGTCCCCTACAGTCAGTTTATAAGGGCTTCAGTAGTGATAGACATTGTTATAAAAACTTGTACCAT
GTTACATGTACACTGCACCACAGTCTAAATTCAGACCAGCATTTGAAGGACTGCACATATTTCATTTCCTCTG
ATTTATGCTGTCACAAATTGAAAATCTGAGTGTAGTTAGGACAAAAAACAAAGCTAACCATATGGCACTAATT
TTGATTTTAACATTAGTGTAACAAATAAGGTCGGGACATATATTTACATAGATCCCAGACACTCAAGAATTAG
CACAGCTGATGTAAAATTCTAAATTACCATTTACTTCCAGATAGAGCAGAGGAGAAACACCTCACTGCAGTTT
CAACATGCTTTCCAAGAACAATATATACGACATATACATTTTCCTGCCTCTCTCTTGTGACAATTTCTGTTAG
AATTTGTTGGGGGTCTGATGGTTAAAATCATGGGCAAGGCCATTCTCTACATCATTGCTTAATATTTCAAAAA
TAGGTATTAGAGCACTATCAGTGGTCCAAAATTAGTTTTAGCTACTTATTTTGCTCCATGGGTTTTGGGCTTT
TCAAAGAATACTATGTATAATTTGTAATTGAAAGCCTTTCAGTAGGATATCCAAAGTTCAATGTGTTTCAAAA
GGGAAAACTTTTACTTGTGGGGGTGGGAGTTAGATGTATTAAAAAAACGAAACTACAAAATCCTTTTAAGAAA
GTAATACCAATTTAGACTCTAGTTTGTGTTACGAGGTATTCTTTCAAATTTTTTTTAAAAGCCAACTACTGTC
ATTAAGAAATAATTTTAGATTTGTGCTCTAGCAGAAAATAACTCTGTACCCATCAATCTTATTGCCAACATTC
CATACCAGTGGTAGGAAAGATATTCTCATTTTTTTTATTGAACCAGACATTTTTATAAAATACTGATTGACTT
TGTACATTATGGATATTATATATGAAATTTTGCCTTGTATTCTTTCACTTGAAAAAACTCCAAAATTCCACAA
TATTTTAGTAGTTAACTTCCTATTCTTTCTTAAATTTGATGGAGAAGGGAAAATAAATTGCAGTTATTGATTC
TTGCCATCTTTGTTTTCTTAAAGAAATCTATGCATTTTAAGGATAAAACTAAAGCATATGGGTTTACATGAAA
CATCAGGATGGATTATTGTACATTGAATTCATTCCGCGTATAATGTGGTATTTCTTACTCTGTCCATCTTGGT
AGCTGTCACTTCAAAAGAAGACAGTTTCCCTGAGTAATCATCACCTACATGGCCATTAGAGTATCTATCGGTG
ATAATTCCATGATACAGAGTATCTTGGCATTATAAATTCAGTATCCCAGGACCTAAACCTTTATGCTACATTT
TCGAAGATTTTTTAAAAATAATATTGTTAATCAGTAAAAAAAAAAAATATTTTTGATCTAAACATAGGTTCTG
CATATCTGTTAGATTTTAAAAATGACTGGTGTTTTGTCTTCACATTTTTGTCTAAGCAAGGAATATAAGATTT
CAAATAAAATTTTGAACCAAAAACATTTATAATGCCGTTCTGGTTTTTCTATTACTTTTTATACTGTACTTTA
AAAGCATTAGGCTGAAAGAGTTTATTTTGGTGGTCAAAAAAAATATGCTTCCACTCATGTAACTATTTTAAAT
GTTAAGAAGTAAAATAATGAAAGATATGTTAATTACTTTATTCAGTAAAGTTTTTTAAGAATGTTTATCTCAG
CCAGTAGGCAAATACTTGGTGTAAAACAAGTTGGTCTTAAATTGTTGCAACTCATTAGCTAGTGAAGATATTC
ATGTAAATATATTTTAGGATTGTAATTAATTTTGAAAATGTAGAAAGCTCTTTATTCCATGTAAGTATGCAGG
ATGCCTGCTTTTTTATGCAGTTGTGGAAAGAATGTAAAATGTTTTAATATATTCTTTGTTAATCTGAGAACTT
GATACTGAATCGTTTATGTGGGAGAAGGAAGGAGTAACATAAAACATGTTTTTATCACTCAAAGTAAGCAATG
GAGGTAACAAATATTGTGCATTTTAACAGTAATATTTGAAGATTTGTAGAATATTCACCTTTAAAACTAGTTA
GTATGCATTTATAATTTTACCAGAATATACAACTAACAATTCAACAGTGATGTTCTTTGCATTTGTGGGGAGA
TGTGTGATGTTCTTGGTTTTCTGGTTTGGAATGGAACGTTTATAGCCTTGCCTGTAAAAATGTGCCCCAGCAC
TTAATGAGTGACCGTTTGAATCCATATGTAGTCCCATTGGTGCTAATGAGAGTAGCTGCTGTGAAACAGGAAT
AAAATGTGTCTGTTCACGGAGGTGCGGTGTGGATGCACCTACAAGGCCAACTCTCTGATCAGGGTGAGGGAGA
GATGGAAGAATTAACAGATCTAAAGTTGGCCTTTGTTGTCTCAAATTGGGTTTGTACACACATGCATGCATGT
GTGTATACAACTGTAATCTGGAACTTCAGGAAGTAAAATTATTTGAAGTTTCTTAGTAAAAAGTAATTAGGTA
TAATTCCAAATAGATTTCTGTATAAATTGTATCTAACTGGATTACAAGTCAGTTTGACAATTTACGATGTCTC
AGTGATGTTTCTGCAATAATTGTGAAATGCATTATTGATTTAAATAATGCTGACACGTACTCTTTAAAAGTGT
TCTTTAACACTGACTACTAAACTTTAAGTTTTAAAAAATAGGTTTTCATTTATGGCTTTTTGTGGTTTTAGCA
CTTTAAGAAGTTTAATTTTTTGTAATTTTTAATATGCTCTTTCAGTGTATCTCAGCGGGTACTCCAGCTTGTG
GCATTCCCATGTGACTGGGGAGAAGACAAAATTGGTGTTTAGATTCTGGAGGAGAAAAAATGAGTTACAATGT
CAGGTGTTTTTAAAAATTCCTTTTAGAGACAGTCTTTTGGGAACCAAAGTATTTTACTGTAAATCTTTAAAAT
AACAGAATTCAGTGCTCTTTCATTTGTTAATGCTGTCCAGATTTTTCACACAGTTATACTCAGTTAATACTAG
TTTTCAGAACATTAGTCCAAGGCTATTTCAAGTTTTCCATTGAATATATGCTTATGGTTAAAAGAAAAACGAA
TGGTTTTATAATTCTGTGTATTTTGTCATATGCCTTAGCAGAGTAAGTCATTTTTCCATTTTACTTAGGAATA
AGCTTTTTCGAACTGAAAATTCATGGTAAAATAGGATTTCTATCTAATGTTAATCCATTGCTTATTTGTATTC
AATTCCATTGTCTACAGTTTGCTTAAATGTTGACTAAGAATGATTGGAGTCAATAAATTTGTACTGTATTTTG
CTTCGA
(SEQ ID NO: 213)
MEGLLHYINPAHAISLLSALNEERLKGQLCDVLLIVGDQKFRAHKNVLAASSEYFQSLFTNKENESQTVFOLD
FCEPDAFDNVLNYIYSSSLFVEKSSLAAVQELGYSLGISELTNIVSKTPQAPFPTCPNRKKVFVEDDENSSQK
RSVIVCQSRNEAQGKTVSQNQPDVSHTSRPSPSIAVKANTNKPHVPKPIEPLHNLSLTEKSWPKDSSVVYAKS
LEHSGSLDDPNRISLVKRNAVLPSKPLQDREAMDDKPGVSGQLPKGKALELALKRPRPPVLSVCSSSETPYLL
KETNKGNGQGEDRNLLYYSKLGLVIPSSGSGSGNQSIDRSGPLVKSLLRRSLSMDSQVPVYSPSIDLKSSQGS
SSVSSDAPGNVLCALSQKSSLKDCSEKTALDDRPQVLQPHRLRSFSASQSTDREGASPVTEVRIKTEPSSPLS
DPSDIIRVTVGDAATTAAASSSSVTRDLSLKTEDDQKDMSRLPAKRRFQADRRLPFKKLKVNEHGSPVSEDNF
EEGSSPTLLDADFPDSDLNKDEFGELEGTRPNKKFKCKHCLKIFRSTAGLHRHVNMYHNPEKPYACDICHKRF
HTNFKVWTHCQTQHGIVKNPSPASSSHAVLDEKFQRKLIDIVREREIKKALIIKLRRGKPGFQGQSSSQAQQV
IKRNLRSRAKGAYICTYCGKAYRFLSQFKQHIKMHPGEKPLGVNKVAKPKEHAPLASPVENKEVYQCRLCNAK
LSSLLEQGSHERLCRNAAVCPYCSLRFFSPELKQEHESKCEYKKLTCLECMRTFKSSFSIWRHQVEVHNQNNM
APTENFSLPVLDHNGDVTGSSRPQSQPEPNKVNHIVTTKDDNVFSDSSEQVNFDSEDSSCLPEDLSLSKQLKI
QVKEEPVEEAEEEAPEASTAPKEAGPSKEASLWPCEKCGKMFTVHKQLERHQELLCSVKPFICHVCNKAFRTN
FRLWSHFQSHMSQASEESAHKESEVCPVPTNSPSPPPLPPPPPLPKIQPLEPDSPTGLSENPTPATEKLFVPQ
ESDTLFYHAPPLSAITFKRQFMCKLCHRTFKTAFSLWSHEQTHN*

An amino acid sequence for the protein encoded by the ZMYND8 gene that is a regulator of T cell exhaustion/dysfunction resistance is available from the UniPROT database at accession no. Q9ULU4, cDNA NM_001281771, with gene location at Chr 20: 47209214-47356889.

```
  (SEQ ID NO: 214)
GGAGCCTGTCCTCCATGTTTTATAAGTATTGACATTACACAGTGTTAACAATGCATCCACAGAGCTTGGCTGA
AGAGGAAATAAAAACAGAACAGGAGGTGGTAGAGGGCATGGATATCTCTACTCGCTCCAAAGATCCTGGCTCT
GCAGAGAGAACAGCCCAGAAAAGAAAGTTCCCCAGCCCTCCACATTCTTCCAATGGCCACTCGCCGCAGGACA
CATCAACAAGCCCCATTAAAAAGAAAAAGAAACCTGGCTTACTGAACAGTAACAATAAGGAGCAGGATGGACG
GAATGATTTCTACTGCTGGGTTTGTCACCGGGAAGGCCAAGTCCTTTGCTGTGAGCTCTGTCCCCGGGTTTAT
CACGCTAAGTGTCTGAGACTGACATCGGAACCAGAGGGGGACTGGTTTTGTCCTGAATGTGAGAAAATTACAG
TAGCAGAATGCATCGAGACCCAGAGTAAAGCCATGACAATGCTCACCATTGAACAGTTATCCTACCTGCTCAA
GTTTGCCATTCAGAAAATGAAACAGCCAGGGACAGATGCATTCCAGAAGCCCGTTCCATTGGAACAGCACCCT
GACTATGCGGAATACATCTTCCATCCAATGGACCTTTGTACATTGGAAAAGAATGCGAAAAAGAAAATGTATG
GCTGCACAGAAGCCTTCCTGGCTGATGCAAAGTGGATTTTGCACAACTGCATCATTTATAATGGGGGAAATCA
CAAATTGACGCAAATAGCGAAAGTAGTCATCAAAATCTGTGAACATGAGATGAATGAAATCGAAGTATGTCCA
GAATGTTATCTAGCTGCTTGCCAAAAACGAGATAACTGGTTTTGTGAGCCTTGTAGCAATCCACATCCTTTGG
TCTGGGCCAAACTGAAGGGGTTTCCATTCTGGCCTGCAAAAGCTCTAAGGGATAAAGACGGGCAGGTCGATGC
CCGATTCTTTGGACAACATGACAGGGCCTGGGTTCCAATAAATAATTGCTACCTCATGTCTAAAGAAATTCCT
TTTTCTGTGAAAAAGACTAAGAGCATCTTCAACAGTGCCATGCAAGAGATGGAGGTTTACGTGGAGAACATCC
GCAGGAAGTTTGGGGTTTTTAATTACTCTCCATTTAGGACACCCTACACACCCAACAGCCAGTATCAAATGCT
GCTCGATCCCACCAACCCCAGCGCCGGCACTGCCAAGATAGACAAGCAGGAGAAGGTCAAGCTCAACTTTGAC
ATGACGGCATCCCCCAAGATCCTGATGAGCAAGCCTGTGCTGAGTGGGGGCACAGGCCGCCGGATTTCCTTGT
CGGATATGCCGCGCTCCCCCATGAGCACAAACTCTTCTGTGCACACGGGCTCCGACGTGGAGCAGGATGCTGA
GAAGAAGGCCACGTCGAGCCACTTCAGTGCGAGCGAGGAGTCCATGGACTTCCTGGATAAGAGCACAGCTTCA
CCAGCCTCCACCAAGACGGGACAAGCAGGGAGTTTATCCGGCAGCCCAAAGCCCTTCTCTCCTCAACTGTCAG
CTCCTATCACGACGAAAACGGACAAAACCTCCACCACCGGCAGCATCCTGAATCTTAACCTGGATCGAAGCAA
AGCTGAGATGGATTTGAAGGAGCTGAGCGAGTCGGTCCAGCAACAGTCCACCCCTGTTCCTCTCATCTCTCCC
AAGCGCCAGATTCGTAGCAGGTTCCAGCTGAATCTTGACAAGACCATAGAGAGTTGCAAAGCACAATTAGGCA
TAAATGAAATCTCGGAAGATGTCTATACGGCCGTAGAGCACAGCGATTCGGAGGATTCTGAGAAGTCAGATAG
TAGCGATAGTGAGTATATCAGTGATGATGAGCAGAAGTCTAAGAACGAGCCAGAAGACACAGAGGACAAAGAA
GGTTGTCAGATGGACAAAGAGCCATCTGCTGTTAAAAAAAAGCCCAAGCCTACAAACCCAGTGGAGATTAAAG
AGGAGCTGAAAAGCACGTCACCAGCCAGCGAGAAGGCAGACCCTGGAGCAGTCAAGGACAAGGCCAGCCCTGA
GCCTGAGAAGGACTTTTCCGAAAAGGCAAAACCTTCACCTCACCCCATAAAGGATAAACTGAAGGGAAAAGAT
GAGACGGATTCCCCAACAGTCCATTTGGGCCTGGACTCTGATTCAGAGAGCGAACTTGTCATAGATTTAGGAG
AAGACCATTCTGGGCGGGAGGGTCGAAAAAATAAGAAGGAACCCAAAGAACCATCTCCCAAACAGGATGCTGT
CCAGCAGAAGGAGATCACACAGAGCCCATCCACGTCCACCATCACCCTGGTGACCAGCACACAGTCATCGCCC
CTGGTCACCAGCTCGGGGTCCATGAGCACCCTTGTGTCCTCAGTCAACGCTGACCTGCCCATCGCCACTGCCT
CAGCTGATGTCGCCGCTGATATTGCCAAGTACACTAGCAAAATGATGGATGCAATAAAAGGAACAATGACAGA
AATATACAACGATCTTTCTAAAAACACTACTGGAAGCACAATAGCTGAGATTCGCAGGCTGAGGATCGAGATA
GAGAAGCTCCAGTGGCTGCACCAGCAAGAGCTCTCCGAAATGAAACACAACTTAGAGCTGACCATGGCGGAGA
TGCGGCAGAGCCTGGAGCAGGAGCGGGACCGGCTCATCGCCGAGGTGAAGAAGCAGCTGGAGTTGGAGAAGCA
GCAGGCGGTGGATGAGACCAAGAAGAAGCAGTGGTGCGCCAACTGCAAGAAGGAGGCCATCTTTTACTGCTGT
TGGAACACCAGCTACTGTGACTACCCCTGCCAGCAAGCCCACTGGCCTGAGCACATGAAGTCCTGCACCCAGT
CAGCTACTGCTCCTCAGCAGGAAGCGGATGCTGAGGTGAACACAGAAACACTAAATAAGTCCTCCCAGGGGAG
CTCCTCGAGCACACAATCAGCACCTTCAGAAACGGCCAGCGCCTCCAAAGAGAAGGAGACGTCAGCTGAGAAA
AGCAAGGAGAGTGGCTCGACCCTTGACCTTTCTGGCTCCAGAGAGACGCCCTCCTCCATTCTCTTAGGCTCCA
ACCAAGGCTCTGACCATTCCCGGAGTAATAAATCCAGTTGGAGCAGCAGTGATGAGAAGAGGGGATCGACACG
TTCCGATCACAACACCAGTACCAGCACGAAGAGCCTCCTCCCGAAAGAGTCTCGGCTGGACACCTTCTGGGAC
TAGCAGTGAATCGGGACACAAACCACCCACCCCATTGGGAGAAAAACCCAGACGCCAGGAAAAGAAGAAACAA
CAAAGGCAGGAGAACAGCCACTTTCAGACTTGAAAATGACAAAACCCTCAGTTGAGCCTGAGCCCCCGGCGCG
GGGGCTGCTACACTACAGGACACCCAGCATCGGCTTTGACTGCAGACTGTTCACCCACACGAGCCCTGTGCTT
TTGGTGTAAATAATGTACAATTTGTGGATGTCATTGAATCTAGAGGACTTTCCCCTTTTTATATTTGTATTAA
CTTTAACTTATTAAAAAAAAAAAAAGAAAAAGAAAAACGATTTAAAAAAAAAAAAAAAAGCAACCAACCCCAA
CAACAAAAAAGAATGTTTTGGTATTGGAGAAGGGATGGTCAGTTAGCCTGTCTGTCACACGACGGAATGGATA
CTGGGCCCGGGGACCACTTTCATACTCACGTCCTCATCCTTGGATACCCAGGGGAGGGCGAACCGTTTTCGCT
CGTGTG
  (SEQ ID NO: 215)
MHPQSLAEEEIKTEQEVVEGMDISTRSKDPGSAERTAQKRKFPSPPHSSNGHSPQDTSTSPIKKKKKPGLLNS
NNKEQDGRNDFYCWVCHREGQVLCCELCPRVYHAKCLRLTSEPEGDWFCPECEKITVAECIETQSKAMTMLTI
EQLSYLLKFAIQKMKQPGTDAFQKPVPLEQHPDYAEYIFHPMDLCTLEKNAKKKMYGCTEAFLADAKWILHNC
IIYNGGNHKLTQIAKVVIKICEHEMNEIEVCPECYLAACQKRDNWFCEPCSNPHPLVWAKLKGFPFWPAKALR
DKDGQVDARFFGQHDRAWVPINNCYLMSKEIPFSVKKTKSIFNSAMQEMEVYVENIRRKFGVFNYSPFRTPYT
PNSQYQMLLDPTNPSAGTAKIDKQEKVKLNFDMTASPKILMSKPVLSGGTGRRISLSDMPRSPMSTNSSVHTG
SDVEQDAEKKATSSHFSASEESMDFLDKSTASPASTKTGQAGSLSGSPKPFSPQLSAPITTKTDKTSTTGSIL
NLNLDRSKAEMDLKELSESVQQQSTPVPLISPKRQIRSRFQLNLDKTIESCKAQLGINEISEDVYTAVEHSDS
EDSEKSDSSDSEYISDDEQKSKNEPEDTEDKEGCQMDKEPSAVKKKPKPTNPVEIKEELKSTSPASEKADPGA
VKDKASPEPEKDFSEKAKPSPHPIKDKLKGKDETDSPTVHLGLDSDSESELVIDLGEDHSGREGRKNKKEPKE
PSPKQDAVQQKEITQSPSTSTITLVTSTQSSPLVTSSGSMSTLVSSVNADLPIATASADVAADIAKYTSKMMD
AIKGTMTEIYNDLSKNTTGSTIAEIRRLRIEIEKLQWLHQQELSEMKHNLELTMAEMRQSLEQERDRLIAEVK
KQLELEKQQAVDETKKKQWCANCKKEAIFYCCWNTSYCDYPCQQAHWPEHMKSCTQSATAPQQEADAEVNTET
LNKSSQGSSSSTQSAPSETASASKEKETSAEKSKESGSTLDLSGSRETPSSILLGSNQGSDHSRSNKSSWSSS
DEKRGSTRSDHNTSTSTKSLLPKESRLDTFWD*
```

An amino acid sequence for the protein encoded by the KIAA1551 gene (also known as RESF1) that is a regulator of T cell exhaustion/dysfunction resistance is available from the UniPROT database at accession no. Q9HCM1, cDNA NM_018169, with gene location at Chr 20: 47209214-47356889.

```
  (SEQ ID NO: 216)
      1 acttgactta aactctgggg cccgggaggc cgccggtttt ctccccgett gccggggtgg
    61 tcctcttccc tttgtcggac caaagaagta aacactgtgt ggagagggac tgacgtgttt
  121 ggagggaaat gggaatgtac acgataatta aagagtaaag ttgtgctcaa cgctgtttac
  181 ttctaattat ttcctgattc acaaattaat ttttgcaatt cacaacttca agaatacttg
  241 gactattgga ttcaatccat agtaatctgc aaaagaagag aagcagaatt atacctagaa
  301 gtctactaat ggacagttag tcacccacct acatgttgag attcctgaca ttcagacaac
  361 tgacttgtaa ctgacttata actgacttgt aatacactgc tactatatca aaccgacaat
  421 gaattggaat gaaaaaccaa agagtgctac attaccacca ctgtatccta aaagccagcc
  481 accttttttg caccagtctt taataaacca aattaccaca acatctcaga gttctttcag
  541 ctatcctgga agtaaccaag aagcatgcat gtatcccggt aattcaaatc caatttcaca
  601 gccactgctg aatatccaaa attatcctca acaaatttct gtttctgata tgcataatgg
  661 gacagttgtg gcctcacaca cttcagtaga aagaataaca tatgcaaatg ttaatggacc
  721 caaacaacta actcacaatt tgcagatgtc ttcaggagtt acccaaaacg tatggttgaa
  781 ctcaccaatg aggaatcctg tgcattctca tataggggca actgtatctc atcaaactga
  841 ttttggagct aacgtaccca atatgccggc actacagagt caactgataa catcagatac
  901 ctattctatg caaatgcaga tgatcccttc taatictaca cgacttcctg tagcttacca
  961 aggaaatcag ggacttaacc agtctttttc agagcaacag gttgattgga cacaacagtg
1021 tatatataag ggactgactt acccagatta cagaccacct ccaaagctat accettactc
1081 accacaaagc tttttaccag attctaccat tcaaaaacaa aactttatac cacatacatc
1141 attgcaagtt aaaaatagtc agcttctaaa ttctgtatta actttaccat caaggcagac
1201 ctcagctgta ccatcacagc agtatgccac gcaaactgac aaaagacctc ctcctcctcc
1261 ttacaactgt agatatggaa gccagccttt gcaaagtact cagcatatta ctaaacactt
1321 gtctatggaa gttcctcaga gtcgagaaat gctgtcatct gaaataagga ccagctttca
1381 acagcagtgg caaaacccta atgaaaatgt cagcacaatt ggaaatttca ctaacttgaa
1441 agtaaatacc aacagcaaac agccttttaa cagtcccatt agatcttctg tggatggtgt
1501 tcagactctt gctcaaacta atgaagagaa aataatggat tcttgcaatc caacttcaaa
1561 tcaagtactg gacacaagtg ttgcaaaaga aaagctagta agggatatta aaacattagt
1621 agagataaaa cagaagtttt cagaacttgc aaggaaaatt aaaatcaata aagatctttt
1681 gatggcagca ggttgtatta aaatgactaa tacticttat agtgaaccag ctcagaattc
1741 taaattgtct ctaaaacaaa ctgccaaaat ccagtctgga ccccagataa ctccagtaat
1801 gccagagaat gcagagagac aaacaccaac agtagtggaa tctgcagaaa caaataagac
1861 tcaatgtatg ttgaattctg acattcagga agtcaattgc agaaggttta accaagttga
1921 ttctgtttta ccaaatcctg tctattctga aaagcggcca atgccagact catctcatga
1981 tgtgaaagtt ctcacttcaa agacatcagc tgttgagatg acccaggcag tattgaatac
2041 tcagctttca tcagaaaatg ttaccaaagt tgagcaaaat tcaccagcag tttgtgaaac
2101 aatttctgtt cccaagtcca tgtccactga ggaatataaa tcaaaaattc aaaatgaaaa
2161 tatgctactt ctcgctttgc tttcacaggc acgtaagact cagaagacag tattaaaaga
2221 tgctaatcaa actattcagg attctaaacc agacagttgt gaaatgaatc caaataccca
2281 aatgactggt aaccaactga atttgaagaa catggaaact ccaagtactt ctaatgtaag
2341 tggcagggtt ttggacaact ccttttgcag tcgacaagaa tectcaacaa aaggaatgcc
2401 tgctaaaagt gacagtagct gttccatgga agtgctagca acctgtcttt ccctgtggaa
2461 aaagcaacct tcagatactg caaaagaaaa ggagtgtgat aaactcagaa caaacacaac
2521 agcagttgga atttcaaagc ctgctaacat ccacgttaag agtccttgtt cagttgtggg
2581 aaattcaaat tctcagaata aaataagtaa tcccccacag cagacagctt tgtcgatggt
2641 aatgcacaat tatgagtctt caggtataaa tataacaaag ggaacagaac ttcagatagc
2701 tgtagtgtca ccgttagttc tgtcagaggt caaaacattg tctgtcaaag gaataacacc
2761 tgcagtgtta cctgaaacag tgtatcccgt tattaaagaa ggcagtgttt gtagtttaca
2821 aaatcaattg gcagaaaatg caaaggcaac tgctgctttg aaagttgatg ttagtggacc
2881 agtagcaagt acagcaacat caaccaagat ttttccacta actcagaagg aaaagcagaa
2941 tgagtcaact aatggtaatt cagaagtcac acctaatgtc aatcaaggaa agcataacaa
3001 attagagtca gctatccatt ctcctatgaa cgatcagcaa atctcacagg agtcaaggaa
3061 tagtactgtt gtgagtagtg atacattaca gattgacaat atttgttctc tggttgaagg
3121 tgatacctct tacaattccc aaatagcaaa gatattcagc tctcttccct tgaaaatggt
3181 tgagccacag aaaccttctc tacccaatca gcaagggatt ggcagcagag aaccagaaaa
3241 acaattagat aataccactg aaaataaaga ctttggtttt caaaaagata aacctgtaca
3301 gtgcacagat gtttcacata aaatatgtga tcagtcaaag tcagagccac ccttagagtc
3361 atcttttaac aatcttgaaa caaacagagt tattctagag aaaagtagtt tggagcatgc
3421 cactgaaaaa agcacagcta acgatacgtg ctcgtcagct gctattcagg aggatattta
3481 ccctcaggaa atagatgcat ccagcaacta tactccccaa gatcctgcaa gaaatgaaat
3541 ccacagtgat aaggcacctg tcttatacct acatgaccag ctgtcagaac ttctaaaaga
3601 gtttccttat ggcattgagg ctgtgaatac acgtgaaggt tctgtgggcc agcaaactac
3661 ataccagacc tcagaagatc aaactgctga taaaaccagt tctgactcca aagacccagc
3721 agatcaaata caaattacaa tattaagctc agagcaaatg aaagaaatat ttcctgaaca
3781 ggatgatcaa ccctatgtag tagacaagtt ggcagaacct cagaaagaag agcccatcac
3841 agaagtagtt agccagtgtg acctgcaggc acctgcagct ggacaaagtc gtgattctgt
3901 gatactggac tctgagaaag atgatatcca ctgctgtgca ttgggctggc tctccatggt
3961 ttacgaagga gtaccccagt gtcagtgtaa ttccatcaag aactcatctt cagaggaaga
4021 gaaacaaaaa gagcagtgtt ctcctttgga taccaacagt tgtaaacaag gagagagaac
4081 ttctgataga gatgtcactg ttgttcaatt taagagcctt gtaaataatc caaagactcc
4141 tccagatggg aaaagtcatt ttcctgaact acaagacgac agtagaaaag atacacccaa
4201 aacaaaacat aaaagcttac caaggacaga acaagaatta gttgctggtc agttttcatc
4261 taaatgtgat aaactaaatc ccttgcaaaa tcacaaaaga aaaaaattga ggtttcacga
4321 ggtaaccttt cactccagta ataaaatgac agcatcttat gaacaagctt ctcaggaaac
4381 ccgacagaag aaacatgtaa cacagaactc acgtccacta aaaacaaaaa cagctttttt
4441 gccaaataaa gatgtgtata agaagcatag ttctttggga cagtcattat caccagaaaa
4501 gataaaattg aaactcaaat cagttagctt caaacaaaaa cgaaagttag accaagggaa
4561 cgtattagat atggaagtaa agaaaaagaa acatgataaa caagaacaga aaggaagtgt
4621 gggagctaca ttcaaattag gtgactcttt gtcaaaccca aacgaaagag ccattgttaa
4681 agaaaagatg gtatcaaata ctaagtctgt agacacgaaa gcgagttcat ctaaatttag
4741 tagaattcta actcctaagg agtatttaca aaggcagaag cataaagaag ctctgagtaa
4801 taaagcatcg aagaaaatct gtgtgaaaaa cgtgccatgt gattctgaac atatgagacc
4861 aagtaaactt gccgtgcagg ttgaaagttg tgggaaatca aatgagaaac acagcagcgg
4921 cgtgcagacc tctaaagaat cattaaatgg cttgacaagc catggtaaaa acctcaaaat
4981 ccaccattct caggagtcta aaacatacaa cattctaagg aatgttaaag aaaaagttgg
5041 tgggaagcag cctgataaaa tatggattga taagactaaa ttagacaaat taaccaatat
5101 aagcaacgaa gctcaattca gccaaatgcc tccccaagta aaggatcaaa agaaattata
5161 tctgaataga gttgggttta aatgcactga acgtgaaagc atttctctca ccaaattaga
5221 aagttcaccc aggaagcttc ataaagataa gagacaggaa aataaacata agaccttttt
5281 accggtgaaa ggtaacacag aaaaatcaaa catgctggag tttaaattat gtccagatat
5341 cttactaaag aatacaaact ctgtggaaga acggaaggat gtaaagcctc atcctaggaa
5401 ggagcaagcc cctctgcaag tttcaggaat aaaaagtaca aaagaagact ggttaaaatt
5461 tgttgctaca aagaaaagga cacagaaaga cagccaagag agagataatg ttaattcaag
5521 actctcgaag agaagcttca gtgcagatgg atttgagatg ctacaaaacc cagtaaaaga
5581 ttcaaaagaa atgtttcaaa cctacaaaca gatgtacctg gagaagagaa gcagaagcct
5641 tggtagcagt cctgtaaaat aattacaaga tgtggttttg taattgccac tgggaaattt
5701 ctttcctttt ctgttcaaaa tatttcgctg aaactaatga gaaatgccat gataaagatt
5761 tctcagagtt tggttcccac tttcattgta tttcattgaa agtgcttaat taaaatggct
5821 tgagaacttt gggtagccat gtgtaagaaa tggatggtat tcaccgggga aacaaggtat
5881 ttgaatttct actttattga accagattta ccattatttt aaaaggaatg cttatacaaa
5941 tcaatttgaa attctaccca tcttgaggga ggaccgttcc tcagttaagg acttgtttat
6001 ttaaatggga ctgtaaatat gttttggttt ctaagctata ttagcaaaat ttatttttca
6061 aaaatgccca ctgtgatgtg aatgtcaaaa tatattctta agtgttttat aactaattgt
6121 aaactttttt tcagaagtct tattttatac ttgtgaaact gaacacaatt ttgggacaac
6181 gtttaaacat tacttttcat acttgaaata aacatttatt ttttaaaaaa ctaa
(SEQ ID NO: 217)
MNWNEKPKSATLPPLYPKSQPPFLHQSLINQITTTSQSSFSYPGSNQEACMYPGNSNPISQPLLNIQNYPQQI
SVSDMHNGTVVASHTSVERITYANVNGPKQLTHNLQMSSGVTQNVWLNSPMRNPVHSHIGATVSHQTDFGANV
PNMPALQSQLITSDTYSMQMQMIPSNSTRLPVAYQGNQGLNQSFSEQQVDWTQQCISKGLTYPDYRPPPKLYR
YSPQSFLPDSTIQKQNFIPHTSLQVKNSQLLNSVLTLPSRQTSAVPSQQYATQTDKRPPPPPYNCRYGSQPLQ
STQHITKHLSMEVPQSREMLSSEIRTSFQQQWQNPNENVSTIGNFTNLKVNTNSKQPENSPIRSSVDGVQTLA
QTNEEKIMDSCNPTSNQVLDTSVAKEKLVRDIKTLVEIKQKFSELARKIKINKDLLMAAGCIKMTNTSYSEPA
QNSKLSLKQTAKIQSGPQITPVMPENAERQTPTVVESAETNKTQCMLNSDIQEVNCRRFNQVDSVLPNPVYSE
KRPMPDSSHDVKVLTSKTSAVEMTQAVLNTQLSSENVTKVEQNSPAVCETISVPKSMSTEEYKSKIQNENMLL
LALLSQARKTQKTVLKDANQTIQDSKPDSCEMNPNTQMTGNQLNLKNMETPSTSNVSGRVLDNSFCSGQESST
KGMPAKSDSSCSMEVLATCLSLWKKQPSDTAKEKECDKLRTNTTAVGISKPANIHVKSPCSVVGNSNSQNKIS
NPSQQTALSMVMHNYESSGINITKGTELQIAVVSPLVLSEVKTLSVKGITPAVLPETVYPVIKEGSVCSLQNQ
LAENAKATAALKVDVSGPVASTATSTKIFPLTQKEKQNESTNGNSEVTPNVNQGKHNKLESAIHSPMNDQQIS
QESRNSTVVSSDTLQIDNICSLVEGDTSYNSQIAKIFSSLPLKMVEPQKPSLPNQQGIGSREPEKQLDNTTEN
KDFGFQKDKPVQCTDVSHKICDQSKSEPPLESSENNLETNRVILEKSSLEHATEKSTANDTCSSAAIQEDIYP
QEIDASSNYTPQDPARNEIHSDKAPVLYLHDQLSELLKEFPYGIEAVNTREGSVGQQTTYQTSEDQTADKTSS
DSKDPADQIQITILSSEQMKEIFPEQDDQPYVVDKLAEPQKEEPITEVVSQCDLQAPAAGQSRDSVILDSEKD
DIHCCALGWLSMVYEGVPQCQCNSIKNSSSEEEKQKEQCSPLDTNSCKQGERTSDRDVTVVQFKSLVNNPKTP
PDGKSHFPELQDDSRKDTPKTKHKSLPRTEQELVAGQFSSKCDKLNPLQNHKRKKLRFHEVTFHSSNKMTASY
EQASQETRQKKHVTQNSRPLKTKTAFLPNKDVYKKHSSLGQSLSPEKIKLKLKSVSFKQKRKLDQGNVLDMEV
KKKKHDKQEQKGSVGATFKLGDSLSNPNERAIVKEKMVSNTKSVDTKASSSKFSRILTPKEYLQRQKHKEALS
NKASKKICVKNVPCDSEHMRPSKLAVQVESCGKSNEKHSSGVQTSKESLNGLTSHGKNLKIHHSQESKTYNIL
RNVKEKVGGKQPDKIWIDKTKLDKLTNISNEAQFSQMPPQVKDQKKLYLNRVGFKCTERESISLTKLESSPRK
LHKDKRQENKHKTFLPVKGNTEKSNMLEFKLCPDILLKNTNSVEERKDVKPHPRKEQAPLQVSGIKSTKEDWL
KFVATKKRTQKDSQERDNVNSRLSKRSFSADGFEMLQNPVKDSKEMFQTYKQMYLEKRSRSLGSSPVK
```

Any of these genes or the proteins encoded by these genes that are described herein can regulate T cell exhaustion/dysfunction resistance.

The sequences provided herein are exemplary. Isoforms and variants of these sequences and of any of regulators listed in Table 1 or FIGS. 1-27 can also be used in the methods and compositions described herein.

For example, isoforms and variants of the proteins and nucleic acids can be used in the methods and compositions described herein when they are substantially identical to the genes, or the encoded proteins listed in Table 1 or FIGS. 1-27. The phrase “substantially identical” indicates that a polypeptide or nucleic acid comprises a sequence with between 55-100% sequence identity to a reference sequence, for example with at least 55% sequence identity, at least 60%, at least 70%, at least 80%, at least 90%, at least 95%, at least 96%, at least 97%, at least 98%, at least 99% sequence identity to a reference sequence over a specified comparison window. Optimal alignment may be ascertained or conducted using the homology alignment algorithm of Needleman and Wunsch, J. Mol. Biol. 48:443-53 (1970).

An indication that two polypeptide sequences are substantially identical is that both polypeptides have the same function—acting as a regulator of T cell exhaustion/dysfunction resistance. The polypeptide that is substantially identical to a regulator sequence and may not have 50 exactly the same level of activity as the regulator. Instead, the substantially identical polypeptide may exhibit greater or lesser levels of regulator activity than the those listed in Table 1 or FIGS. 1-27, or any of the sequences recited herein. For example, the substantially identical polypeptide or nucleic acid may have at least about 40%, or at least about 50%, or at least about 60%, or at least about 70%, or at least about 80%, or at least about 90%, or at least about 95%, or at least about 97%, or at least about 98%, or at least about 100%, or at least about 105%, or at least about 110%, or at least about 120%, or at least about 130%, or at least about 140%, or at least about 150%, or at least about 200% of the activity of a regulator described herein when measured by similar assay procedures.

Alternatively, substantial identity is present when a second polypeptide is immunologically reactive with antibodies raised against the first polypeptide (e.g., a polypeptide encoded by any of the genes listed in Table 1 or FIGS. 1-27). Thus, a polypeptide is substantially identical to a first polypeptide, for example, where the two polypeptides differ only by a conservative substitution. In addition, a polypeptide can be substantially identical to a first polypeptide when they differ by a non-conservative change if the epitope that the antibody recognizes is substantially identical. Polypeptides that are “substantially similar” share sequences as noted above except that some residue positions, which are not identical, may differ by conservative amino acid changes.

### Expression Systems

Nucleic acid segments encoding one or more regulator proteins, or nucleic acid segments that are inhibitory nucleic acids or such regulators, can be inserted into or employed with any suitable expression system. Nucleic acids segments encoding one or more agents that can modulate a regulator protein expression or activity can be inserted into or employed with any suitable expression system. A therapeutically effective quantity of one or more regulator proteins or modulators of such regulator proteins can be generated from such expression systems. A therapeutically effective of one or more inhibitory nucleic acids can also be generated from such expression systems.

Recombinant expression of nucleic acids (or inhibitory nucleic acids) is usually accomplished using a vector, such as a plasmid. The vector can include a promoter operably linked to nucleic acid segment encoding one or more regulator/modulator proteins. In another example, a vector can include a promoter operably linked to nucleic acid segment that encodes a regulator/modulator inhibitory nucleic acid.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying, encoding and/or expressing a regulator/modulator can be employed. A variety of prokaryotic and eukaryotic expression vectors suitable for carrying, encoding and/or expressing regulator/modulator inhibitory nucleic acids can be employed. Such expression vectors include, for example, pET, pET3d, pCR2.1, pBAD, pUC, and yeast vectors. The vectors can be used, for example, in a variety of in vivo, ex vivo and/or in vitro situations.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms; the cDNA sequences may be expressed in either a sense (to produce mRNA) or anti-sense orientation (to produce an anti-sense RNA transcript that is complementary to the mRNA transcript). Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding a regulator protein. In another example, the promoter can be upstream of an inhibitory nucleic acid segment of a modulating agent for one or more regulators.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of regulator/modulator proteins or inhibitory nucleic acid molecules therefor from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if the gene has been delivered to the cell and once delivered is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid. (Mulligan, R. C. and Berg, P. Science 209: 1422 (1980)) or hygromycin. (Sugden. B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the nucleic acid molecules, expression cassette and/or vectors encoding regulator/modulator proteins or encoding inhibitory nucleic acid molecules therefor can be introduced to a cell by any method including, but not limited to, calcium-mediated transformation, electroporation, microinjection, lipofection, particle bombardment and the like. The cells can be expanded in culture and then administered to a subject, e.g., a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors encoding one or more regulator/modulator. In some cases, the transgenic cell can produce exosomes or microvesicles that contain inhibitory nucleic acid molecules that can target regulator/modulator nucleic acids, one or more nucleic acids for regulator, or a combination thereof. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem. 286(16): 14383-14395 (2011); Hu et al., Frontiers in Genetics 3 (April 2012); Pegtel et al., Proc. Nat'l Acad Sci 107(14): 6328-6333 (2010); WO/2013/084000; each of which is incorporated herein by reference in its entirety. Cells producing such microvesicles can be used to express the one or more regulator/modulator protein and/or inhibitory nucleic acids for one or more regulator/modulators, or a combination thereof.

Transgenic vectors or cells with a heterologous expression cassette or expression vector can express one or more regulator, can optionally also express one or more regulator inhibitory nucleic acids, or a combination thereof. Any of these vectors or cells can be administered to a subject. Exosomes produced by transgenic cells can be used to administer regulator/modulator proteins, regulator/modulator nucleic acids, regulator/modulator inhibitory nucleic acids, or a combination thereof to a subject or to tumor and cancer cells in the subject.

Methods and compositions that include inhibitors of one or regulators such as inhibitory nucleic acids, antibodies, or any combination thereof.

### Crispr Modifications

In some cases, clustered regularly interspaced short palindromic repeats (CRISPR)/CRISPR-associated (Cas) systems can be used to create one or more modifications in regulator genes. Such CRISPR modifications can reduce or activate the expression or functioning of the regulator gene products. CRISPR/Cas systems are useful, for example, for RNA-programmable genome editing (see e.g., Marraffini and Sontheimer. Nature Reviews Genetics 11: 181-190 (2010); Sorek et al. Nature Reviews Microbiology 2008 6: 181-6; Karginov and Hannon. Mol Cell 2010 1:7-19: Hale et al. Mol Cell 2010:45:292-302: Jinek et al. Science 2012 337:815-820; Bikard and Marraffini Curr Opin Immunol 2012 24:15-20: Bikard et al. Cell Host & Microbe 2012 12: 177-186; all of which are incorporated by reference herein in their entireties).

A CRISPR guide RNA can be used that can target a Cas enzyme to the desired location in the genome, where it can cleave the genomic DNA for generation of a genomic modification. This technique is described, for example, by Mali et al. Science 2013 339:823-6; which is incorporated by reference herein in its entirety. Kits for the design and use of CRISPR-mediated genome editing are commercially available, e.g., the PRECISION X CAS9 SMART NUCLEASE™ System (Cat No. CAS900A-1) from System Biosciences, Mountain View, CA.

In some cases, transcriptional activators can be linked to defective Cas9 or to one or more guide RNAs to target the transcriptional activator. Such transcriptional activators include protein domains or whole proteins that assist in the recruitment of co-factors and RNA Polymerase to increase transcription of one or more of the regulator gene(s) listed in Table 1 or FIGS. 1-27.

In some cases, a cre-lox recombination system of bacteriophage P1, described by Abremski et al. 1983. Cell 32:1301 (1983), Steinberg et al., Cold Spring Harbor Symposia on Quantitative Biology, Vol. XLV 297 (1981) and others, can be used to promote recombination and alteration of the regulator genomic site(s). The cre-lox system utilizes the cre recombinase isolated from bacteriophage P1 in conjunction with the DNA sequences that the recombinase recognizes (termed lox sites). This recombination system has been effective for achieving recombination in plant cells (see, e.g., U.S. Pat. No. 5,658,772), animal cells (U.S. Pat. Nos. 4,959,317 and 5,801,030), and in viral vectors (Hardy et al., J. Virology 71:1842 (1997).

The genomic mutations so incorporated can alter one or more amino acids in the encoded regulator gene products. For example, genomic sites modified so that the encoded regulator protein is more prone to degradation, is less stable so that the half-life of such protein(s) is reduced, or so that the regulator has improved expression or functioning. In another example, genomic sites can be modified so that at least one amino acid of a regulator polypeptide is deleted or mutated to alter its activity. For example, a conserved amino acid or a conserved domain can be modified to improve or reduce of the activity of the regulator polypeptide. For example, a conserved amino acid or several amino acids in a conserved domain of the regulator polypeptide can be replaced with one or more amino acids having physical and/or chemical properties that are different from the conserved amino acid(s). For example, to change the physical and/or chemical properties of the conserved amino acid(s), the conserved amino acid(s) can be deleted or replaced by amino acid(s) of another class, where the classes are identified in the following table.

| Classification | Genetically Encoded |
|---|---|
| Hydrophobic | A, G, F, I, L, M, P, V, W |
| Aromatic | F, Y, W |
| Apolar | M, G, P |
| Aliphatic | A, V, L, I |
| Hydrophilic | C, D, E, H, K, N, Q, R, S, T, Y |
| Acidic | D, E |
| Basic | H, K, R |
| Polar | Q, N, S, T, Y |
| Cysteine-Like | C |

The guide RNAs and nuclease can be introduced via one or more vehicles such as by one or more expression vectors (e.g., viral vectors), virus like particles, ribonucleoproteins (RNPs), via nanoparticles, liposomes, or a combination thereof. The vehicles can include components or agents that can target particular cell types (e.g., antibodies that recognize cell-surface markers), facilitate cell penetration, reduce degradation, or a combination thereof.

### Inhibitory Nucleic Acids

The expression of one or more regulators/modulators can be inhibited, for example by use of an inhibitory nucleic acid that specifically recognizes a nucleic acid that encodes the regulator or modulator.

An inhibitory nucleic acid can have at least one segment that will hybridize to a regulator nucleic acid or modulator under intracellular or stringent conditions. The inhibitory nucleic acid can reduce expression of a regulator/modulator nucleic acid. A nucleic acid may hybridize to a genomic DNA, a messenger RNA, or a combination thereof. An inhibitory nucleic acid may be incorporated into a plasmid vector or viral DNA. It may be single stranded or double stranded, circular or linear.

As an example, an inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a regulator/modulator nucleic acid. Such an inhibitory nucleic acid may be completely complementary to a segment of an endogenous regulator/modulator nucleic acid (e.g., an RNA). Alternatively, some variability is permitted in the inhibitory nucleic acid sequences relative to regulator/modulator sequences. An inhibitory nucleic acid can hybridize to a regulator/modulator nucleic acid under intracellular conditions or under stringent hybridization conditions and is sufficiently complementary to inhibit expression of the endogenous regulator/modulator nucleic acid. Intracellular conditions refer to conditions such as temperature, pH and salt concentrations typically found inside a cell, e.g., an animal or mammalian cell. One example of such an animal or mammalian cell is a myeloid progenitor cell. Another example of such an animal or mammalian cell is a more differentiated cell derived from a myeloid progenitor cell. Generally, stringent hybridization conditions are selected to be about 5° C. lower than the thermal melting point (Tm) for the specific sequence at a defined ionic strength and pH. However, stringent conditions encompass temperatures in the range of about PC to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a regulator/modulator coding sequence, each separated by a stretch of contiguous nucleotides that are not complementary to adjacent coding sequences, can inhibit the function of one or more nucleic acids for any of the regulators or modulators described herein. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g., a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of regulator/modulator such that translation of the encoded regulator/modulator polypeptide is reduced. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the regulator/modulator transcript and/or any of the transcripts of the regulators/modulators. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting expression of regulators/modulators. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected preferably 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19)UU, where N is any nucleotide in the mRNA sequence and should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/mai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target regulators/modulators nucleic acids.

An inhibitory nucleic acid may be prepared using available methods, for example, by expression from an expression vector encoding a complementarity sequence of the regulator/modulator nucleic acids described herein. Alternatively, it may be prepared by chemical synthesis using naturally occurring nucleotides, modified nucleotides or any mixture of combination thereof. In some embodiments, the nucleic acids of the regulators/modulators described herein are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, the regulator/modulator nucleic acids can be peptide nucleic acids that have peptide bonds rather than phosphodiester bonds.

Naturally occurring nucleotides that can be employed in the regulator/modulator nucleic acids include the ribose or deoxyribose nucleotides adenosine, guanine, cytosine, thymine and uracil. Examples of modified nucleotides that can be employed in the regulators/modulator nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil. 2-methylthio-N6-isopentenyladenine, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, inhibitory nucleic acids of the regulators/modulators described herein may include modified nucleotides, as well as natural nucleotides such as combinations of ribose and deoxyribose nucleotides. The inhibitory nucleic acids and may be of same length as wild type regulators/modulators described herein. The inhibitory nucleic acids of the regulators/modulators described herein can also be longer and include other useful sequences. In some embodiments, the inhibitory nucleic acids of the regulators/modulators described herein are somewhat shorter. For example, inhibitory nucleic acids of the regulators-/modulators described herein can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

### Antibodies

Antibodies can be used as inhibitors or activators of any of the regulators/modulators described herein. For example, in some cases, antibody preparations can target one or more of the regulators or modulators described herein to block interactions by the regulators/modulators described herein or to reduce the activities or the regulators/modulators. In other cases, for example, antibodies can activate one or more of the regulator or modulators described herein that are cell surface receptors. One example of such activation is Varlilumab (a CD27 activating antibody) currently in clinical trial and that has been shown to increase anti-tumor T cell function Ansell et al. (2020) Blood Adv. 4(9): 1917-1926.

Antibodies can be raised against various epitopes of the regulators/modulators described herein. Some antibodies for regulators/modulators described herein may also be available commercially. However, the antibodies contemplated for treatment pursuant to the methods and compositions described herein are preferably human or humanized antibodies and are highly specific for their targets.

In one aspect, the present disclosure relates to use of isolated antibodies that bind specifically to regulators/modulators described herein. Such antibodies may be monoclonal antibodies. Such antibodies may also be humanized or fully human monoclonal antibodies. The antibodies can exhibit one or more desirable functional properties, such as high affinity binding to one or more regulators/modulators described herein, or the ability to inhibit functioning of any of the regulators/modulators described herein.

Methods and compositions described herein can include antibodies that bind any of the regulators/modulators described herein, or a combination of antibodies where each antibody type can separately bind one of the regulators/modulators described herein.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., a peptide or domain of any of the regulators/modulators described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and CHI domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv): see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds any of the regulators/modulators described herein is substantially free of antibodies that specifically bind antigens other than any of the regulators/modulators described herein). An isolated antibody that specifically binds regulators/modulators described herein may, however, have cross-reactivity to other antigens, such as isoforms or related forms of the regulators/modulators proteins from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to a human regulator/modulator protein described herein” is intended to refer to an antibody that binds to the human regulator/modulator protein described herein with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}.” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to a human regulator/modulator described herein. Preferably, an antibody of the invention binds to a regulator/modulator described herein with high affinity, for example with a K_{D} of 1×10⁻⁷ M or less. The antibodies can exhibit one or more of the following characteristics:
(a) binds to a human regulator/modulator described herein with a K_{D} of 1×10⁻⁷ M or less;
(b) inhibits the function or activity of a human regulator/modulator described herein;
(c) inhibits cancer (e.g., metastatic cancer); or
(d) a combination thereof.

Assays to evaluate the binding ability of the antibodies toward a human regulator/modulator described herein can be used, including for example, ELISAs, Western blots and RIAs. The binding kinetics (e.g., binding affinity) of the antibodies also can be assessed by standard assays known in the art, such as by Biacore™, analysis.

Given that each of the subject antibodies can bind to a human regulator/modulator described herein, the V_{L} and V_{H} sequences can be “mixed and matched” to create other binding molecules that bind to a human regulator/modulator described herein. The binding properties of such “mixed and matched” antibodies can be tested using the binding assays described above and assessed in assays described in the examples. When V_{L} and V_{H} chains are mixed and matched, a V_{H} sequence from a particular V_{H}/V_{L} pairing can be replaced with a structurally similar V_{H} sequence. Likewise, preferably a V_{L} sequence from a particular V_{H}/V_{L} pairing is replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds a human regulator/modulator described herein.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody): Rader et al., Proc. Natl. Acad. Sci. U.S.A. 95:8910-8915 (1998) (describing a panel of humanized anti-integrin alphaᵥbeta₃ antibodies using a heavy and light chain variable CDR3 domain. Hence, in some cases a mixed and matched antibody or a humanized antibody contains a CDR3 antigen binding domain that is specific for any of the regulators/modulators described herein.

### Assays for Drug Development/Treating

Methods are also described herein for evaluating whether test agents can modulate the expression or activity of any of the regulators/modulators described herein. T cells, cancer cells, and combinations thereof can be evaluated for susceptibility to treatment with candidate compounds.

Specifically, the methods can include assay steps for identifying a candidate test agent that selectively modulates the proliferation, functioning, or viability of T cells or cancer cells, or for increasing or decreasing the levels or functioning of regulators described herein. For example, if the proliferation, cytokine production activity, or viability of T cells is increased or decreased in the presence of one or more of the regulators described herein but the proliferation, cytokine production, activity, or the proliferation, activity, or viability of the T cells in the T cell-regulator assay mixture changes in the presence of a test agent then that test agent has utility for modulating the regulator of the T cells. Such a test agent is referred to as a, modulator.

An assay can include determining whether a test agent can specifically cause decreased or increased numbers of T cells or whether a compound can specifically cause decreased or increased functioning of T cells. If the test agent does cause altered T cell numbers or T cell functioning, then the test agent can be selected/identified for further study, such as for its suitability as a therapeutic agent to treat a cancer or an immune condition or disease. For example, the test agent identified by the selection methods featured in the invention can be further examined for their ability to target a tumor, target an immune cell, or to treat cancer by, for example, administering the test agent (modulator) to an animal model.

The cells that are evaluated can include cytotoxic T cells, helper T cells, regulatory T cells, naive T cells, activated T cells, CD4 T cells, CD8 T cells, metastatic cells, benign cell samples, cell lines (including as cancer cell lines), or a combination thereof. The cells that are evaluated can also include cells from a patient with cancer (including a patient with metastatic cancer), or cells from a known cancer type or cancer cell line, or cells exhibiting an overproduction of any of the regulators described herein. A test agent that can modulate the production or activity of any of these cell types can be administered to an animal, including a patient.

For example, one method, can include (a) obtaining a cell sample from a patient: (b) measuring the amount or concentration of T cells/regulators/modulators in a known number or weight of cells from the sample to generate a reference value: (c) mixing the known number or weight of cells from the sample with a test agent to generate a test assay: (d) measuring the amount or numbers of T cells, regulators or modulators in the test assay to generate a test assay T cell/regulator/modulator value: (e) optionally repeating steps (c) and (d) with separate samples; and (f) selecting a test agent with a lower or higher a test assay T cell/regulator/modulator value than the reference value. The method can further include administering a test agent to an animal model, for example, to further evaluate the toxicity and/or efficacy of the test agent. In some cases, the method can further include administering the test agent to the patient from whom the cell or tissue sample as obtained.

Test agents or modulators (e.g., top hits identified by any method described herein) can be used in a cell-based assay using T cells or cells that express any of the regulators described herein as a readout of the efficacy of the test agents or modulators.

Assay methods are also described herein for identifying and assessing the potency of agents that may modulate T cell exhaustion/dysfunction resistance of any of the regulators listed in Table 1 or FIGS. 1-27.

For example, T cells can release cytokines, such as Interferon γ or Interleukin-2. T cells or T cells expressing any of the modulators described herein can be contacted with a test agent and the release of cytokines by the T-cells can be measured. Such a test agent-related level of cytokines can be compared to the level observed for T cells not contacted with a test agent.

Useful test regulators, modulators, and test agents can be administered to a test animal or a patient.

“Treatment” or “treating” refers to both therapeutic treatment and to prophylactic or preventative measures. Those in need of treatment include those already with the disorder as well as those prone to have the disorder, or those in whom the disorder is to be prevented.

“Subject” for purposes of administration of a regulator, modulator, test agent or composition described herein refers to administration to any animal classified as a mammal or bird, including humans, domestic animals, farm animals, zoo animals, experimental animals, pet animals, such as dogs, horses, cats, cows, etc. The experimental animals can include mice, rats, guinea pigs, goats, dogs, monkeys, or a combination thereof. In some cases, the subject is human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, lung, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

In some cases, a hematological cancer or hematological malignancy can be treated. The term “hematological malignancies” includes adult or childhood leukemia and lymphomas, Hodgkin's disease, lymphomas of lymphocytic and cutaneous origin, acute and chronic leukemia, plasma cell neoplasm, and cancers associated with AIDS.

The inventive methods and compositions can also be used to treat leukemias, lymph nodes, thymus tissues, tonsils, spleen, cancer of the breast, cancer of the lung, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. In some cases, metastatic cancers are treated but primary cancers are not treated. Information regarding numerous types of cancer can be found, e.g., from the American Cancer Society (cancer.org), or from, e.g., Wilson et al. (1991) Harrison's Principles of Internal Medicine, 12th Edition, McGraw-Hill. Inc.

In some embodiments, the cancer and/or tumors to be treated are hematological malignancies, or those of lymphoid origin such as cancers or tumors of lymph nodes, thymus tissues, tonsils, spleen, and cells related thereto. In some embodiments, the cancer and/or tumors to be treated are those that have been resistant to T cell therapies.

Treatment of, or treating, metastatic cancer can include the reduction in cancer cell migration or the reduction in establishment of at least one metastatic tumor. The treatment also includes alleviation or diminishment of more than one symptom of metastatic cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent metastatic cancer, it may substantially eliminate metastatic tumor formation and growth, and/or it may arrest or inhibit the migration of metastatic cancer cells.

Anti-cancer activity can reduce the progression of a variety of cancers (e.g., breast, lung, pancreatic, or prostate cancer) using methods available to one of skill in the art. Anti-cancer activity, for example, can determined by identifying the lethal dose (LD₁₀₀) or the 50% effective dose (ED50) or the dose that inhibits growth at 50% (GI₅₀) of an agent of the present invention that prevents the migration of cancer cells. In one aspect, anti-cancer activity is the amount of the agent that reduces 50%, 60%, 70%, 80%, 90%, 95%, 97%, 98%, 99% or 100% of cancer cell migration, for example, when measured by detecting expression of a cancer cell marker at sites proximal or distal from a primary tumor site, or when assessed using available methods for detecting metastases.

In another example, agents that increase or decrease regulator/modulator expression or function can be administered to sensitize tumor cells to immune therapies. Hence, by administering an agent that increase or decrease regulator/modulator expression or function, tumor cells can become more sensitive to the immune system and to various immune therapies.

### Compositions

The invention also relates to compositions containing one or more active agents such as any of the regulators described herein, modulators described herein, or combinations thereof. Such active agents can be a polypeptide, a nucleic acid encoding a polypeptide (e.g., within an expression cassette or expression vector), a modified cell, an inhibitory nucleic acid, a small molecule, a compound identified by a method described herein, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include a protein or polypeptide encoded by any of the genes listed in Table 1 or FIGS. 1-27. In other embodiments, the compositions can include at least one nucleic acid or expression cassette encoding a polypeptide listed in Table 1 or FIGS. 1-27. In other embodiments, the compositions can include at least one nucleic acid or expression cassette that includes a nucleic acid segment complementarity to a gene listed in Table 1 (e.g., an inhibitory nucleic acid). In other embodiments, the compositions can include at least one nucleic acid or expression cassette that includes a nucleic acid segment encoding a cas nuclease and at least one guide RNA that can target a regulator or modulator described herein. In other embodiments, the compositions can include at least one antibody that binds at least one protein encoded by at least one gene listed in Table 1 or FIGS. 1-27. In other embodiments, the compositions can include at least one small molecule that binds, that activates, or that inhibits at least one gene listed in Table 1 or FIGS. 1-27, or at least one small molecule that binds, that activates, or that inhibits at least one protein encoded by at least one gene listed in Table 1 or FIGS. 1-27. In other embodiments, the compositions can include cells with at least one modified genomic regulator or modulator genetic site, cells that express one or more of the regulators described herein, cells that express a cas nuclease and at least one guide RNA that can target at least one regulator or modulator gene, cells that express one or more inhibitory nucleic acids, or a combination thereof. The cells can be immune cells. In some cases, the cells can be one or more types of lymphoid cells, myeloid cells, cytotoxic T cells, helper T cells, regulatory T cells, naive T cells, activated T cells, CD4 T cells, CD8 T cells, gamma delta T cells, chimeric antigen receptor (CAR) cells, natural killer (NK) cells, induced pluripotent stem cell-derived immune (e.g., lymphoid and/or myeloid) cells, or a combination thereof.

The amount or number of cells administered can vary but amounts in the range of about 10⁶ to about 10⁹ cells can be used. The cells are generally delivered in a physiological solution such as saline or buffered saline. The cells can also be delivered in a vehicle such as within a population of liposomes, exosomes or microvesicles.

In some embodiments, the active agents of the invention (e.g., polypeptide, a nucleic acid encoding a polypeptide (e.g., within an expression cassette or expression vector), an antibody, an inhibitory nucleic acid, a small molecule, a compound identified by a method described herein, modified cells, or a combination thereof), are administered in a “therapeutically effective amount.” Such a therapeutically effective amount is an amount sufficient to obtain the desired physiological effect, such a reduction of at least one symptom of disease.

The disease can be cancer or an immune disease or condition. For example, active agents can reduce the symptoms of disease by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%. For example, symptoms of cancer can also include tumor cachexia, tumor-induced pain conditions, tumor-induced fatigue, tumor growth, and metastatic spread. Hence, the active agents may also reduce tumor cachexia, tumor-induced pain conditions, tumor-induced fatigue, tumor growth, or a combination thereof by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, peptides, or nucleic acid chosen for administration, the disease, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated.

To prepare the composition, small molecules, compounds, polypeptides, nucleic acids, expression cassettes, ribonucleoprotein complexes, and other agents are synthesized or otherwise obtained, purified as necessary or desired. These small molecules, compounds, polypeptides, nucleic acids, expression cassettes, ribonucleoprotein complexes, and other agents can be suspended in a pharmaceutically acceptable carrier and/or lyophilized or otherwise stabilized. The small molecules, compounds, polypeptides, nucleic acids, expression cassettes, ribonucleoprotein complexes, other agents, and combinations thereof can be adjusted to an appropriate concentration, and optionally combined with other agents. The absolute weight of a given small molecule, compound, polypeptide, nucleic acid, ribonucleoprotein complex, and/or other agents included in a unit dose can vary widely. For example, about 0.01 to about 2 g, or about 0.1 to about 500 mg, of at least one molecule, compound, polypeptide, nucleic acid, ribonucleoprotein complexes, and/or other agent, or a plurality of molecules, compounds, polypeptides, nucleic acids, ribonucleoprotein complexes, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the active agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of active agent for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the active agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The active agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the active agent with liquid carriers, solid matrices, semi-solid carriers, finely divided solid carriers or combinations thereof, and then, if necessary, introducing or shaping the product into the desired delivery system. For example, the active agent(s) can be linked to a convenient carrier such as a nanoparticle, albumin, polyalkylene glycol, or be supplied in prodrug form. The active agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the active agent(s) and/or other agents can sometimes be administered in an oral dosage form, that oral dosage form can be formulated so as to protect the small molecules, compounds, polypeptides, nucleic acids, expression cassettes, ribonucleoprotein complexes, and combinations thereof from degradation or breakdown before the small molecules, compounds, polypeptides, nucleic acids encoding such polypeptides, expression cassettes, ribonucleoprotein complexes, and combinations thereof provide therapeutic utility. For example, in some cases the small molecules, compounds, polypeptides, nucleic acids encoding such polypeptide, expression cassettes, ribonucleoprotein complexes, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The active agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

An active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as active agents, anti-viral agents, antibacterial agents, antimicrobial agents and/or preservatives. Examples of additional therapeutic agents that may be used include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compositions can also be used in conjunction with radiation therapy.

The present description is further illustrated by the following examples, which should not be construed as limiting in any way. The contents of all cited references (including literature references, issued patents, published patent applications as cited throughout this application) are hereby expressly incorporated by reference.

### EXAMPLES

### Example 1

### Methods

Human T cells were sourced from PBMC-enriched leukapheresis products (Leukopaks, STEMCELL Technologies, catalog no. 70500.2) from healthy donors, after institutional review board-approved informed written consent (STEMCELL Technologies). CD8+ T cells were isolated from Leukopaks using EasySep magnetic selection following the manufacturer's recommended protocol (STEMCELL Technologies, catalog no. 17953).

CD8+ T cells were cultured in X-VIVO 15 (Lonza Bioscience, catalog no. 04-418Q) supplemented with 5% fetal calf serum (FCS), and 100 IU/ml of recombinant human IL-2 (Amerisource Bergen, catalog no. 10101641). Primary CD8+ T cells were activated using anti-human CD3/CD28 CTS Dynabeads (Fisher Scientific, catalog no. 40203D) at a 1:1 cell:bead ratio at 10⁶ cells/ml.

Eighteen hours after activation, CD8+ T cells from two human blood donors were transduced with concentrated mCherry-2A-dCas9-VP64 (CRISPRa), or mCherry-2A-ZIM3-dCas9 (CRISPRi) lentivirus. Eight hours following dCas9 transduction, cells were transduced with either genome-wide CRISPRa library Calabrese (Addgene, catalog nos. 92379/92380) or genome-wide CRISPRi library Dolcetto (Addgene, catalog nos. 92385/92386). 16 hours following library transductions, cells were split into two groups and transduced with chimeric antigen receptor CAR lentiviruses; anti-CD19-28-zeta-2A-GFP, or high-affinity (HA) anti-GD2-28-zeta-2A-GFP. Three days post-activation puromycin was added to select for library transduced cells. After two days of puromycin selection cells were sorted for mCherry+(dCas9+) and GFP+(CAR+) cells by fluorescence activated cell sorting (FACS).

Cells were passaged every 2-3 days as needed, adding medium to maintain densities at 10⁶ cells/ml, adding IL-2 to 100 IU/ml at each passage. At least 100-fold library coverage was maintained at each passage. On days 9 and 18 post-activation T cells were re-stimulated with NALM6-GD2 target cells (CD19+/GD2+) at 1:1 effector to target ratio. At each passage cells were sampled at >=100-fold library coverage for downstream genomic DNA extraction and sgRNA PCR.

Genomic DNA was extracted from cells as described previously (Freimer et al. Nat Genet. 2022 August; 54(8):1133-1144. doi: 10.1038/s41588-022-01106-y. Epub 2022 Jul. 11; PMID:35817986) Integrated sgRNA sequences were amplified as described previously (Sanson et al. Nat Commun. 2018 Dec. 21; 9(1):5416. doi: 10.1038/s41467-018-07901-8; PMID:30575746), and sequencing libraries were subsequently agarose gel purified using NucleoSpin Gel and PCR Clean-up Mini kit (Machery-Nagel, catalog no. 740609.50). Libraries from days 5, 13, 18, and 22 post-activation were sequenced on a NextSeq500 instrument to a minimum targeted depth of 100-fold coverage.

Reads were aligned to the appropriate reference library using MAGeCK version 0.5.9.2 (PMID:25476604) using the count command with -trim-5 22,23,24,25,26,28,29,30 argument to remove the staggered 5′ adapter. Statistics were generated with MAGeCK test command for the following comparisons: (1) HAGD2-CAR days 18/22 versus day 5 (HAGD2-CAR time), (2) CD19-CAR days 18/22 versus day 5 (CD19-CAR time), (3) HAGD2-CAR days 18/22 versus CD19-CAR days 18/22 (HAGD2-vs-CD19). For each comparison significant genes were defined as having a false discovery rate (FDR)>0.1 in the positive enrichment tests. Hits were defined as being significant in the HAGD2-CAR time test and either a hit in the HAGD2-vs-CD19 or not a hit in the CD19-CAR time test.

The CRISPRa and CRISPRi screens discovered 37 and 69 genes (FDR<0.1), respectively, that when upregulated or downregulated caused exhaustion/dysfunction resistance (Table 1).

TABLE 1

| | | | pos \| | pos \| | pos \| | pos \| |
|---|---|---|---|---|---|---|
| CRISPRa.or.i | comparison.test | gene | p-value | fdr | rank | lfc |
| CRISPRa | GD2 CAR | FOXL2NB | 2.62E−07 | 4.50E−04 | 1 | 6.0517 |
| | Abundance (Late | | | | | |
| | vs Early) | | | | | |
| CRISPRa | GD2 CAR | FOXO6 | 2.62E−07 | 4.50E−04 | 2 | 3.6955 |
| | Abundance (Late | | | | | |
| | vs Early) | | | | | |
| CRISPRa | GD2 CAR | IRF4 | 2.62E−07 | 4.50E−04 | 3 | 3.4857 |
| | Abundance (Late | | | | | |
| | vs Early) | | | | | |
| CRISPRa | GD2 CAR | STAT5A | 2.62E−07 | 4.50E−04 | 4 | 2.295 |
| | Abundance (Late | | | | | |
| | vs Early) | | | | | |
| CRISPRa | GD2 CAR | IL2RA | 2.62E−07 | 4.50E−04 | 5 | 2.166 |
| | Abundance (Late | | | | | |
| | vs Early) | | | | | |
| CRISPRa | GD2 CAR | GLIS3 | 2.62E−07 | 4.50E−04 | 6 | 2.0829 |
| | Abundance (Late | | | | | |
| | vs Early) | | | | | |
| CRISPRa | GD2 CAR | BATF | 2.62E−07 | 4.50E−04 | 7 | 2.1192 |
| | Abundance (Late | | | | | |
| | vs Early) | | | | | |
| CRISPRa | GD2 CAR | FOXO4 | 2.62E−07 | 4.50E−04 | 8 | 2.6448 |
| | Abundance (Late | | | | | |
| | vs Early) | | | | | |
| CRISPRa | GD2 CAR | IL2RB | 2.62E−07 | 4.50E−04 | 9 | 2.1364 |
| | Abundance (Late | | | | | |
| | vs Early) | | | | | |
| CRISPRa | GD2 CAR | ITPKA | 2.62E−07 | 4.50E−04 | 10 | 2.1802 |
| | Abundance (Late | | | | | |
| | vs Early) | | | | | |
| CRISPRa | GD2 CAR | EOMES | 2.62E−07 | 4.50E−04 | 11 | 1.6566 |
| | Abundance (Late | | | | | |
| | vs Early) | | | | | |
| CRISPRa | GD2 CAR | IL10 | 1.83E−06 | 0.002666 | 12 | 1.3509 |
| | Abundance (Late | | | | | |
| | vs Early) | | | | | |
| CRISPRa | GD2 CAR | PTEN | 1.31E−06 | 0.002063 | 13 | 2.0867 |
| | Abundance (Late | | | | | |
| | vs Early) | | | | | |
| CRISPRa | GD2 CAR | GLI2 | 2.35E−06 | 0.003182 | 14 | 2.2368 |
| | Abundance (Late | | | | | |
| | vs Early) | | | | | |
| CRISPRa | GD2 CAR | TRAF3IP2 | 7.06E−06 | 0.008911 | 15 | 1.7376 |
| | Abundance (Late | | | | | |
| | vs Early) | | | | | |
| CRISPRa | GD2 CAR | FOSL1 | 9.15E−06 | 0.010829 | 16 | 1.2591 |
| | Abundance (Late | | | | | |
| | vs Early) | | | | | |
| CRISPRa | GD2 CAR | FOXL2 | 1.12E−05 | 0.012522 | 17 | 1.7211 |
| | Abundance (Late | | | | | |
| | vs Early) | | | | | |
| CRISPRa | GD2 CAR | LIFR | 1.39E−05 | 0.01433 | 18 | 1.6697 |
| | Abundance (Late | | | | | |
| | vs Early) | | | | | |
| CRISPRa | GD2 CAR | ZNF211 | 1.44E−05 | 0.01433 | 19 | 0.68556 |
| | Abundance (Late | | | | | |
| | vs Early) | | | | | |
| CRISPRa | GD2 CAR | FOSB | 2.17E−05 | 0.019566 | 20 | 1.4773 |
| | Abundance (Late | | | | | |
| | vs Early) | | | | | |
| CRISPRa | GD2 CAR | NPM3 | 2.17E−05 | 0.019566 | 21 | 1.503 |
| | Abundance (Late | | | | | |
| | vs Early) | | | | | |
| CRISPRa | GD2 CAR | SHOC2 | 3.32E−05 | 0.028578 | 22 | 1.143 |
| | Abundance (Late | | | | | |
| | vs Early) | | | | | |
| CRISPRa | GD2 CAR | S100A13 | 4.21E−05 | 0.034653 | 23 | −0.22005 |
| | Abundance (Late | | | | | |
| | vs Early) | | | | | |
| CRISPRa | GD2 CAR | TMEM167B | 4.47E−05 | 0.035272 | 24 | 1.0315 |
| | Abundance (Late | | | | | |
| | vs Early) | | | | | |
| CRISPRa | GD2 CAR | LCK | 5.62E−05 | 0.040937 | 25 | 0.75049 |
| | Abundance (Late | | | | | |
| | vs Early) | | | | | |
| CRISPRa | GD2 CAR | IL9R | 5.62E−05 | 0.040937 | 26 | 1.0022 |
| | Abundance (Late | | | | | |
| | vs Early) | | | | | |
| CRISPRa | GD2 CAR | CUL3 | 6.35E−05 | 0.044554 | 27 | 1.2708 |
| | Abundance (Late | | | | | |
| | vs Early) | | | | | |
| CRISPRa | GD2 CAR | INPP5D | 6.90E−05 | 0.046676 | 28 | 0.98522 |
| | Abundance (Late | | | | | |
| | vs Early) | | | | | |
| CRISPRa | GD2 CAR | NQO1 | 1.05E−04 | 0.067605 | 29 | 1.0236 |
| | Abundance (Late | | | | | |
| | vs Early) | | | | | |
| CRISPRa | GD2 CAR | PLEKHH3 | 1.13E−04 | 0.067605 | 30 | 1.6227 |
| | Abundance (Late | | | | | |
| | vs Early) | | | | | |
| CRISPRa | GD2 CAR | TSR2 | 1.14E−04 | 0.067605 | 31 | 1.2021 |
| | Abundance (Late | | | | | |
| | vs Early) | | | | | |
| CRISPRa | GD2 CAR | CLASRP | 1.18E−04 | 0.067957 | 32 | −0.045175 |
| | Abundance (Late | | | | | |
| | vs Early) | | | | | |
| CRISPRa | GD2 CAR | ITPKB | 1.23E−04 | 0.068579 | 33 | 0.97166 |
| | Abundance (Late | | | | | |
| | vs Early) | | | | | |
| CRISPRa | GD2 CAR | VAV1 | 1.38E−04 | 0.07454 | 34 | 0.8347 |
| | Abundance (Late | | | | | |
| | vs Early) | | | | | |
| CRISPRa | GD2 CAR | RFLNB | 1.13E−04 | 0.067605 | 35 | 0.71374 |
| | Abundance (Late | | | | | |
| | vs Early) | | | | | |
| CRISPRa | GD2 CAR | LYN | 1.57E−04 | 0.082371 | 36 | 1.371 |
| | Abundance (Late | | | | | |
| | vs Early) | | | | | |
| CRISPRa | GD2 CAR | CDKL3 | 1.79E−04 | 0.091383 | 37 | −0.03913 |
| | Abundance (Late | | | | | |
| | vs Early) | | | | | |
| CRISPRa | GD2 CAR | TRMT44 | 9.01E−04 | 0.279581 | 61 | 0.033711 |
| | Abundance (Late | | | | | |
| | vs Early) | | | | | |
| CRISPRa | GD2 CAR | HOXB13 | 0.0028046 | 0.421381 | 124 | 0.9794 |
| | Abundance (Late | | | | | |
| | vs Early) | | | | | |
| CRISPRa | GD2 CAR | WAS | 0.0031587 | 0.434896 | 134 | 0.8214 |
| | Abundance (Late | | | | | |
| | vs Early) | | | | | |
| CRISPRa | GD2 CAR | KLLN | 0.0064437 | 0.538383 | 224 | 1.1315 |
| | Abundance (Late | | | | | |
| | vs Early) | | | | | |
| CRISPRa | CD19 CAR | IRF4 | 4.21E−05 | 0.183168 | 4 | 0.84928 |
| | Abundance (Late | | | | | |
| | vs Early) | | | | | |
| CRISPRa | CD19 CAR | NQO1 | 7.71E−05 | 0.190594 | 6 | 1.1993 |
| | Abundance (Late | | | | | |
| | vs Early) | | | | | |
| CRISPRa | CD19 CAR | ITPKA | 0.0024767 | 0.66343 | 69 | 0.75072 |
| | Abundance (Late | | | | | |
| | vs Early) | | | | | |
| CRISPRa | CD19 CAR | BATF | 0.0030745 | 0.674547 | 85 | 0.836 |
| | Abundance (Late | | | | | |
| | vs Early) | | | | | |
| CRISPRa | CD19 CAR | FOSL1 | 0.02337 | 0.842593 | 517 | −0.008423 |
| | Abundance (Late | | | | | |
| | vs Early) | | | | | |
| CRISPRa | CD19 CAR | TRMT44 | 0.023871 | 0.843108 | 529 | −0.59179 |
| | Abundance (Late | | | | | |
| | vs Early) | | | | | |
| CRISPRa | CD19 CAR | IL2RA | 0.024264 | 0.846826 | 534 | 0.4588 |
| | Abundance (Late | | | | | |
| | vs Early) | | | | | |
| CRISPRa | CD19 CAR | TSR2 | 0.037475 | 0.876525 | 800 | 0.53591 |
| | Abundance (Late | | | | | |
| | vs Early) | | | | | |
| CRISPRa | CD19 CAR | STAT5A | 0.047125 | 0.889642 | 991 | 0.58375 |
| | Abundance (Late | | | | | |
| | vs Early) | | | | | |
| CRISPRa | CD19 CAR | IL9R | 0.04864 | 0.889642 | 1023 | 0.34374 |
| | Abundance (Late | | | | | |
| | vs Early) | | | | | |
| CRISPRa | CD19 CAR | LIFR | 0.060521 | 0.899629 | 1263 | 0.1627 |
| | Abundance (Late | | | | | |
| | vs Early) | | | | | |
| CRISPRa | CD19 CAR | TRAF3IP2 | 0.060829 | 0.899629 | 1269 | 0.3436 |
| | Abundance (Late | | | | | |
| | vs Early) | | | | | |
| CRISPRa | CD19 CAR | ZNF211 | 0.079776 | 0.917706 | 1627 | 0.2213 |
| | Abundance (Late | | | | | |
| | vs Early) | | | | | |
| CRISPRa | CD19 CAR | EOMES | 0.094751 | 0.930243 | 1908 | −0.82963 |
| | Abundance (Late | | | | | |
| | vs Early) | | | | | |
| CRISPRa | CD19 CAR | IL10 | 0.11113 | 0.935544 | 2219 | 0.46186 |
| | Abundance (Late | | | | | |
| | vs Early) | | | | | |
| CRISPRa | CD19 CAR | S100A13 | 0.11544 | 0.935544 | 2310 | 0.036239 |
| | Abundance (Late | | | | | |
| | vs Early) | | | | | |
| CRISPRa | CD19 CAR | CDKL3 | 0.17405 | 0.960475 | 3406 | −0.31789 |
| | Abundance (Late | | | | | |
| | vs Early) | | | | | |
| CRISPRa | CD19 CAR | FOSB | 0.17565 | 0.960798 | 3438 | 0.34676 |
| | Abundance (Late | | | | | |
| | vs Early) | | | | | |
| CRISPRa | CD19 CAR | GLIS3 | 0.20728 | 0.966849 | 4038 | 0.25755 |
| | Abundance (Late | | | | | |
| | vs Early) | | | | | |
| CRISPRa | CD19 CAR | SHOC2 | 0.24628 | 0.981523 | 4722 | −0.10855 |
| | Abundance (Late | | | | | |
| | vs Early) | | | | | |
| CRISPRa | CD19 CAR | LCK | 0.27229 | 0.987987 | 5190 | 0.18388 |
| | Abundance (Late | | | | | |
| | vs Early) | | | | | |
| CRISPRa | CD19 CAR | FOXL2NB | 0.27464 | 0.988831 | 5239 | −0.89072 |
| | Abundance (Late | | | | | |
| | vs Early) | | | | | |
| CRISPRa | CD19 CAR | LYN | 0.28622 | 0.990105 | 5449 | 0.080476 |
| | Abundance (Late | | | | | |
| | vs Early) | | | | | |
| CRISPRa | CD19 CAR | HOXB13 | 0.38648 | 1 | 7172 | −0.58138 |
| | Abundance (Late | | | | | |
| | vs Early) | | | | | |
| CRISPRa | CD19 CAR | IL2RB | 0.39917 | 1 | 7392 | 0.13749 |
| | Abundance (Late | | | | | |
| | vs Early) | | | | | |
| CRISPRa | CD19 CAR | CLASRP | 0.42864 | 1 | 7922 | −0.15441 |
| | Abundance (Late | | | | | |
| | vs Early) | | | | | |
| CRISPRa | CD19 CAR | WAS | 0.50862 | 1 | 9355 | −1.3472 |
| | Abundance (Late | | | | | |
| | vs Early) | | | | | |
| CRISPRa | CD19 CAR | FOXO6 | 0.51743 | 1 | 9529 | −0.51883 |
| | Abundance (Late | | | | | |
| | vs Early) | | | | | |
| CRISPRa | CD19 CAR | CUL3 | 0.5228 | 1 | 9636 | −0.15685 |
| | Abundance (Late | | | | | |
| | vs Early) | | | | | |
| CRISPRa | CD19 CAR | FOXL2 | 0.69698 | 1 | 12854 | −0.71876 |
| | Abundance (Late | | | | | |
| | vs Early) | | | | | |
| CRISPRa | CD19 CAR | NPM3 | 0.71241 | 1 | 13151 | −0.44266 |
| | Abundance (Late | | | | | |
| | vs Early) | | | | | |
| CRISPRa | CD19 CAR | ITPKB | 0.77927 | 1 | 14473 | 0.228 |
| | Abundance (Late | | | | | |
| | vs Early) | | | | | |
| CRISPRa | CD19 CAR | GLI2 | 0.86724 | 1 | 16259 | −0.071686 |
| | Abundance (Late | | | | | |
| | vs Early) | | | | | |
| CRISPRa | CD19 CAR | PTEN | 0.88188 | 1 | 16505 | −0.64299 |
| | Abundance (Late | | | | | |
| | vs Early) | | | | | |
| CRISPRa | CD19 CAR | PLEKHH3 | 0.92066 | 1 | 17227 | −0.28972 |
| | Abundance (Late | | | | | |
| | vs Early) | | | | | |
| CRISPRa | CD19 CAR | RFLNB | 0.96222 | 1 | 18079 | −0.36439 |
| | Abundance (Late | | | | | |
| | vs Early) | | | | | |
| CRISPRa | CD19 CAR | TMEM167B | 0.97524 | 1 | 18355 | −0.57958 |
| | Abundance (Late | | | | | |
| | vs Early) | | | | | |
| CRISPRa | CD19 CAR | INPP5D | 0.98673 | 1 | 18580 | −0.30181 |
| | Abundance (Late | | | | | |
| | vs Early) | | | | | |
| CRISPRa | CD19 CAR | VAV1 | 0.98979 | 1 | 18661 | −1.3953 |
| | Abundance (Late | | | | | |
| | vs Early) | | | | | |
| CRISPRa | CD19 CAR | KLLN | 0.99476 | 1 | 18782 | −0.79962 |
| | Abundance (Late | | | | | |
| | vs Early) | | | | | |
| CRISPRa | CD19 CAR | FOXO4 | 0.99997 | 1 | 18930 | −2.1051 |
| | Abundance (Late | | | | | |
| | vs Early) | | | | | |
| CRISPRa | GD2 CAR vs CD19 | FOXL2NB | 2.62E−07 | 9.90E−04 | 1 | 7.3568 |
| | CAR (Late | | | | | |
| | Timepoints) | | | | | |
| CRISPRa | GD2 CAR vs CD19 | FOXO6 | 2.62E−07 | 9.90E−04 | 2 | 3.6108 |
| | CAR (Late | | | | | |
| | Timepoints) | | | | | |
| CRISPRa | GD2 CAR vs CD19 | FOXO4 | 2.62E−07 | 9.90E−04 | 3 | 4.7811 |
| | CAR (Late | | | | | |
| | Timepoints) | | | | | |
| CRISPRa | GD2 CAR vs CD19 | EOMES | 2.62E−07 | 9.90E−04 | 4 | 2.232 |
| | CAR (Late | | | | | |
| | Timepoints) | | | | | |
| CRISPRa | GD2 CAR vs CD19 | IRF4 | 2.62E−07 | 9.90E−04 | 5 | 2.9741 |
| | CAR (Late | | | | | |
| | Timepoints) | | | | | |
| CRISPRa | GD2 CAR vs CD19 | FOXL2 | 4.97E−06 | 0.013437 | 6 | 2.6298 |
| | CAR (Late | | | | | |
| | Timepoints) | | | | | |
| CRISPRa | GD2 CAR vs CD19 | IL2RA | 4.97E−06 | 0.013437 | 7 | 2.1679 |
| | CAR (Late | | | | | |
| | Timepoints) | | | | | |
| CRISPRa | GD2 CAR vs CD19 | HOXB13 | 7.06E−06 | 0.015347 | 8 | 1.5123 |
| | CAR (Late | | | | | |
| | Timepoints) | | | | | |
| CRISPRa | GD2 CAR vs CD19 | KLLN | 8.11E−06 | 0.015347 | 9 | 2.0409 |
| | CAR (Late | | | | | |
| | Timepoints) | | | | | |
| CRISPRa | GD2 CAR vs CD19 | IL2RB | 1.33E−05 | 0.022952 | 10 | 2.1246 |
| | CAR (Late | | | | | |
| | Timepoints) | | | | | |
| CRISPRa | GD2 CAR vs CD19 | PTEN | 7.32E−06 | 0.015347 | 11 | 2.6207 |
| | CAR (Late | | | | | |
| | Timepoints) | | | | | |
| CRISPRa | GD2 CAR vs CD19 | CUL3 | 2.59E−05 | 0.040842 | 12 | 1.8886 |
| | CAR (Late | | | | | |
| | Timepoints) | | | | | |
| CRISPRa | GD2 CAR vs CD19 | TRMT44 | 3.79E−05 | 0.055217 | 13 | 0.63134 |
| | CAR (Late | | | | | |
| | Timepoints) | | | | | |
| CRISPRa | GD2 CAR vs CD19 | RFLNB | 4.13E−05 | 0.05587 | 14 | 0.53646 |
| | CAR (Late | | | | | |
| | Timepoints) | | | | | |
| CRISPRa | GD2 CAR vs CD19 | INPP5D | 6.51E−05 | 0.082178 | 15 | 1.7893 |
| | CAR (Late | | | | | |
| | Timepoints) | | | | | |
| CRISPRa | GD2 CAR vs CD19 | S100A13 | 7.45E−05 | 0.088181 | 16 | −0.32981 |
| | CAR (Late | | | | | |
| | Timepoints) | | | | | |
| CRISPRa | GD2 CAR vs CD19 | WAS | 7.98E−05 | 0.088818 | 17 | 2.1546 |
| | CAR (Late | | | | | |
| | Timepoints) | | | | | |
| CRISPRa | GD2 CAR vs CD19 | VAV1 | 8.60E−05 | 0.090484 | 18 | 2.2124 |
| | CAR (Late | | | | | |
| | Timepoints) | | | | | |
| CRISPRa | GD2 CAR vs CD19 | NPM3 | 1.01E−04 | 0.100834 | 19 | 1.8164 |
| | CAR (Late | | | | | |
| | Timepoints) | | | | | |
| CRISPRa | GD2 CAR vs CD19 | GLI2 | 1.09E−04 | 0.103218 | 20 | 1.9646 |
| | CAR (Late | | | | | |
| | Timepoints) | | | | | |
| CRISPRa | GD2 CAR vs CD19 | ZNF211 | 1.36E−04 | 0.122348 | 21 | 1.4239 |
| | CAR (Late | | | | | |
| | Timepoints) | | | | | |
| CRISPRa | GD2 CAR vs CD19 | STAT5A | 1.52E−04 | 0.125484 | 22 | 1.4424 |
| | CAR (Late | | | | | |
| | Timepoints) | | | | | |
| CRISPRa | GD2 CAR vs CD19 | ITPKA | 2.04E−04 | 0.148324 | 25 | 1.3751 |
| | CAR (Late | | | | | |
| | Timepoints) | | | | | |
| CRISPRa | GD2 CAR vs CD19 | GLIS3 | 2.65E−04 | 0.167492 | 30 | 1.6781 |
| | CAR (Late | | | | | |
| | Timepoints) | | | | | |
| CRISPRa | GD2 CAR vs CD19 | FOSB | 3.53E−04 | 0.20237 | 33 | 1.6741 |
| | CAR (Late | | | | | |
| | Timepoints) | | | | | |
| CRISPRa | GD2 CAR vs CD19 | CDKL3 | 3.91E−04 | 0.210534 | 34 | 0.53482 |
| | CAR (Late | | | | | |
| | Timepoints) | | | | | |
| CRISPRa | GD2 CAR vs CD19 | CLASRP | 6.12E−04 | 0.276403 | 39 | 0.067064 |
| | CAR (Late | | | | | |
| | Timepoints) | | | | | |
| CRISPRa | GD2 CAR vs CD19 | TMEM167B | 8.83E−04 | 0.310139 | 51 | 1.414 |
| | CAR (Late | | | | | |
| | Timepoints) | | | | | |
| CRISPRa | GD2 CAR vs CD19 | PLEKHH3 | 9.70E−04 | 0.326993 | 55 | 1.5438 |
| | CAR (Late | | | | | |
| | Timepoints) | | | | | |
| CRISPRa | GD2 CAR vs CD19 | LIFR | 0.001074 | 0.344605 | 59 | 0.85166 |
| | CAR (Late | | | | | |
| | Timepoints) | | | | | |
| CRISPRa | GD2 CAR vs CD19 | SHOC2 | 0.0013643 | 0.3743 | 69 | 1.1815 |
| | CAR (Late | | | | | |
| | Timepoints) | | | | | |
| CRISPRa | GD2 CAR vs CD19 | FOSL1 | 0.001915 | 0.4265 | 85 | 1.1054 |
| | CAR (Late | | | | | |
| | Timepoints) | | | | | |
| CRISPRa | GD2 CAR vs CD19 | BATF | 0.0030609 | 0.499276 | 111 | 0.92451 |
| | CAR (Late | | | | | |
| | Timepoints) | | | | | |
| CRISPRa | GD2 CAR vs CD19 | TRAF3IP2 | 0.0034181 | 0.500756 | 125 | 1.17 |
| | CAR (Late | | | | | |
| | Timepoints) | | | | | |
| CRISPRa | GD2 CAR vs CD19 | IL10 | 0.005895 | 0.606516 | 183 | 1.49 |
| | CAR (Late | | | | | |
| | Timepoints) | | | | | |
| CRISPRa | GD2 CAR vs CD19 | ITPKB | 0.0096513 | 0.720495 | 249 | 1.0508 |
| | CAR (Late | | | | | |
| | Timepoints) | | | | | |
| CRISPRa | GD2 CAR vs CD19 | LYN | 0.010237 | 0.731091 | 264 | 0.95793 |
| | CAR (Late | | | | | |
| | Timepoints) | | | | | |
| CRISPRa | GD2 CAR vs CD19 | TSR2 | 0.068751 | 0.937125 | 1375 | 0.5019 |
| | CAR (Late | | | | | |
| | Timepoints) | | | | | |
| CRISPRa | GD2 CAR vs CD19 | LCK | 0.12509 | 0.979525 | 2396 | 0.55557 |
| | CAR (Late | | | | | |
| | Timepoints) | | | | | |
| CRISPRa | GD2 CAR vs CD19 | NQO1 | 0.25357 | 0.999484 | 4769 | 0.13742 |
| | CAR (Late | | | | | |
| | Timepoints) | | | | | |
| CRISPRa | GD2 CAR vs CD19 | IL9R | 0.35451 | 0.999979 | 6633 | 0.11288 |
| | CAR (Late | | | | | |
| | Timepoints) | | | | | |
| CRISPRi | GD2 CAR | MED12 | 2.61E−07 | 8.25E−04 | 1 | 2.9426 |
| | Abundance (Late | | | | | |
| | vs Early) | | | | | |
| CRISPRi | GD2 CAR | MED24 | 2.61E−07 | 8.25E−04 | 2 | 2.3794 |
| | Abundance (Late | | | | | |
| | vs Early) | | | | | |
| CRISPRi | GD2 CAR | ELOB | 2.61E−07 | 8.25E−04 | 3 | 1.791 |
| | Abundance (Late | | | | | |
| | vs Early) | | | | | |
| CRISPRi | GD2 CAR | MED13 | 2.61E−07 | 8.25E−04 | 4 | 1.287 |
| | Abundance (Late | | | | | |
| | vs Early) | | | | | |
| CRISPRi | GD2 CAR | RNF7 | 2.61E−07 | 8.25E−04 | 5 | 2.0741 |
| | Abundance (Late | | | | | |
| | vs Early) | | | | | |
| CRISPRi | GD2 CAR | CCNC | 2.61E−07 | 8.25E−04 | 6 | 2.0715 |
| | Abundance (Late | | | | | |
| | vs Early) | | | | | |
| CRISPRi | GD2 CAR | ELOC | 3.40E−06 | 0.009194 | 7 | 1.2128 |
| | Abundance (Late | | | | | |
| | vs Early) | | | | | |
| CRISPRi | GD2 CAR | SYCE1L | 7.58E−06 | 0.017052 | 8 | 1.1361 |
| | Abundance (Late | | | | | |
| | vs Early) | | | | | |
| CRISPRi | GD2 CAR | ATF7IP2 | 8.10E−06 | 0.017052 | 9 | 1.5047 |
| | Abundance (Late | | | | | |
| | vs Early) | | | | | |
| CRISPRi | GD2 CAR | SYTL2 | 1.02E−05 | 0.019307 | 10 | 1.2432 |
| | Abundance (Late | | | | | |
| | vs Early) | | | | | |
| CRISPRi | GD2 CAR | PRDM14 | 1.54E−05 | 0.026553 | 11 | 1.1576 |
| | Abundance (Late | | | | | |
| | vs Early) | | | | | |
| CRISPRi | GD2 CAR | NQO2 | 1.99E−05 | 0.031353 | 12 | 1.0288 |
| | Abundance (Late | | | | | |
| | vs Early) | | | | | |
| CRISPRi | GD2 CAR | SRRD | 3.06E−05 | 0.044554 | 13 | 1.0681 |
| | Abundance (Late | | | | | |
| | vs Early) | | | | | |
| CRISPRi | GD2 CAR | IGFL4 | 4.31E−05 | 0.054455 | 14 | 0.70046 |
| | Abundance (Late | | | | | |
| | vs Early) | | | | | |
| CRISPRi | GD2 CAR | SPIN1 | 4.31E−05 | 0.054455 | 15 | 1.4964 |
| | Abundance (Late | | | | | |
| | vs Early) | | | | | |
| CRISPRi | GD2 CAR | UBE2E1 | 5.78E−05 | 0.068379 | 16 | 1.2381 |
| | Abundance (Late | | | | | |
| | vs Early) | | | | | |
| CRISPRi | GD2 CAR | MN1 | 6.77E−05 | 0.074505 | 17 | 0.94298 |
| | Abundance (Late | | | | | |
| | vs Early) | | | | | |
| CRISPRi | GD2 CAR | MED1 | 7.19E−05 | 0.074505 | 18 | 1.5292 |
| | Abundance (Late | | | | | |
| | vs Early) | | | | | |
| CRISPRi | GD2 CAR | KIAA1551 | 7.50E−05 | 0.074505 | 19 | 1.2184 |
| | Abundance (Late | | | | | |
| | vs Early) | | | | | |
| CRISPRi | GD2 CAR | MEOX1 | 7.87E−05 | 0.074505 | 20 | 0.99518 |
| | Abundance (Late | | | | | |
| | vs Early) | | | | | |
| CRISPRi | GD2 CAR | MOCS3 | 8.91E−05 | 0.080387 | 21 | −0.11395 |
| | Abundance (Late | | | | | |
| | vs Early) | | | | | |
| CRISPRi | GD2 CAR | MED19 | 1.12E−04 | 0.096535 | 22 | 0.98363 |
| | Abundance (Late | | | | | |
| | vs Early) | | | | | |
| CRISPRi | GD2 CAR | UBAP2L | 1.89E−04 | 0.119307 | 30 | 1.348 |
| | Abundance (Late | | | | | |
| | vs Early) | | | | | |
| CRISPRi | GD2 CAR | ARHGAP5 | 3.67E−04 | 0.170611 | 39 | 0.99407 |
| | Abundance (Late | | | | | |
| | vs Early) | | | | | |
| CRISPRi | GD2 CAR | PDCL | 4.83E−04 | 0.212871 | 43 | 0.91418 |
| | Abundance (Late | | | | | |
| | vs Early) | | | | | |
| CRISPRi | GD2 CAR | CAND1 | 7.42E−04 | 0.263935 | 52 | 1.2963 |
| | Abundance (Late | | | | | |
| | vs Early) | | | | | |
| CRISPRi | GD2 CAR | PIK3R5 | 0.0016114 | 0.331737 | 92 | 0.7121 |
| | Abundance (Late | | | | | |
| | vs Early) | | | | | |
| CRISPRi | GD2 CAR | COPS7B | 0.0021378 | 0.35622 | 112 | 1.0581 |
| | Abundance (Late | | | | | |
| | vs Early) | | | | | |
| CRISPRi | GD2 CAR | DCUN1D3 | 0.0042555 | 0.475677 | 168 | 0.6781 |
| | Abundance (Late | | | | | |
| | vs Early) | | | | | |
| CRISPRi | GD2 CAR | ZMYND8 | 0.0056528 | 0.491229 | 217 | 0.79917 |
| | Abundance (Late | | | | | |
| | vs Early) | | | | | |
| CRISPRi | GD2 CAR | CASP3 | 0.0069947 | 0.523963 | 250 | 0.74159 |
| | Abundance (Late | | | | | |
| | vs Early) | | | | | |
| CRISPRi | GD2 CAR | CBFB | 0.0092661 | 0.571661 | 307 | 0.49309 |
| | Abundance (Late | | | | | |
| | vs Early) | | | | | |
| CRISPRi | GD2 CAR | CDKN2A | 0.010555 | 0.593317 | 334 | 0.11609 |
| | Abundance (Late | | | | | |
| | vs Early) | | | | | |
| CRISPRi | GD2 CAR | ARIH2 | 0.013739 | 0.645397 | 401 | 0.67785 |
| | Abundance (Late | | | | | |
| | vs Early) | | | | | |
| CRISPRi | GD2 CAR | MED23 | 0.015404 | 0.649033 | 439 | 0.63034 |
| | Abundance (Late | | | | | |
| | vs Early) | | | | | |
| CRISPRi | GD2 CAR | UBE2L3 | 0.016092 | 0.652649 | 465 | 0.75702 |
| | Abundance (Late | | | | | |
| | vs Early) | | | | | |
| CRISPRi | GD2 CAR | CUL5 | 0.021043 | 0.695407 | 567 | 0.50595 |
| | Abundance (Late | | | | | |
| | vs Early) | | | | | |
| CRISPRi | GD2 CAR | RPS6KA1 | 0.026899 | 0.723709 | 691 | 0.45284 |
| | Abundance (Late | | | | | |
| | vs Early) | | | | | |
| CRISPRi | GD2 CAR | UBE2F | 0.032155 | 0.723709 | 824 | 0.66653 |
| | Abundance (Late | | | | | |
| | vs Early) | | | | | |
| CRISPRi | GD2 CAR | KRTAP10-9 | 0.036118 | 0.723709 | 940 | 0.28258 |
| | Abundance (Late | | | | | |
| | vs Early) | | | | | |
| CRISPRi | GD2 CAR | MYB | 0.036677 | 0.723709 | 953 | 0.66622 |
| | Abundance (Late | | | | | |
| | vs Early) | | | | | |
| CRISPRi | GD2 CAR | NFKBIA | 0.051646 | 0.739919 | 1317 | 0.24236 |
| | Abundance (Late | | | | | |
| | vs Early) | | | | | |
| CRISPRi | GD2 CAR | MAPKAP1 | 0.069723 | 0.795714 | 1655 | 0.60013 |
| | Abundance (Late | | | | | |
| | vs Early) | | | | | |
| CRISPRi | GD2 CAR | SND1 | 0.11108 | 0.874066 | 2401 | 0.10428 |
| | Abundance (Late | | | | | |
| | vs Early) | | | | | |
| CRISPRi | GD2 CAR | KDM5C | 0.14344 | 0.900048 | 3002 | 0.29829 |
| | Abundance (Late | | | | | |
| | vs Early) | | | | | |
| CRISPRi | GD2 CAR | PAK2 | 0.15527 | 0.915227 | 3198 | −0.17378 |
| | Abundance (Late | | | | | |
| | vs Early) | | | | | |
| CRISPRi | GD2 CAR | WDFY1 | 0.16779 | 0.924842 | 3418 | 0.23697 |
| | Abundance (Late | | | | | |
| | vs Early) | | | | | |
| CRISPRi | GD2 CAR | DDA1 | 0.16996 | 0.927439 | 3455 | 0.25216 |
| | Abundance (Late | | | | | |
| | vs Early) | | | | | |
| CRISPRi | GD2 CAR | CISH | 0.20033 | 0.937164 | 4028 | 0.28205 |
| | Abundance (Late | | | | | |
| | vs Early) | | | | | |
| CRISPRi | GD2 CAR | CDK13 | 0.2034 | 0.937164 | 4088 | 0.32304 |
| | Abundance (Late | | | | | |
| | vs Early) | | | | | |
| CRISPRi | GD2 CAR | RNASE9 | 0.20733 | 0.937164 | 4170 | 0.22583 |
| | Abundance (Late | | | | | |
| | vs Early) | | | | | |
| CRISPRi | GD2 CAR | MADD | 0.22466 | 0.941802 | 4495 | 0.11272 |
| | Abundance (Late | | | | | |
| | vs Early) | | | | | |
| CRISPRi | GD2 CAR | CFAP54 | 0.28774 | 0.961372 | 5652 | 0.092835 |
| | Abundance (Late | | | | | |
| | vs Early) | | | | | |
| CRISPRi | GD2 CAR | BORCS8 | 0.29786 | 0.968496 | 5812 | 0.045025 |
| | Abundance (Late | | | | | |
| | vs Early) | | | | | |
| CRISPRi | GD2 CAR | RASA2 | 0.31292 | 0.969483 | 6087 | −0.25412 |
| | Abundance (Late | | | | | |
| | vs Early) | | | | | |
| CRISPRi | GD2 CAR | ATF7IP | 0.31476 | 0.969483 | 6121 | 0.24421 |
| | Abundance (Late | | | | | |
| | vs Early) | | | | | |
| CRISPRi | GD2 CAR | TSPEAR | 0.33153 | 0.973136 | 6434 | −0.005439 |
| | Abundance (Late | | | | | |
| | vs Early) | | | | | |
| CRISPRi | GD2 CAR | CPSF4L | 0.42658 | 0.995487 | 8099 | −0.038094 |
| | Abundance (Late | | | | | |
| | vs Early) | | | | | |
| CRISPRi | GD2 CAR | MYO5A | 0.43256 | 0.996044 | 8205 | −0.61462 |
| | Abundance (Late | | | | | |
| | vs Early) | | | | | |
| CRISPRi | GD2 CAR | ST8SIA5 | 0.54516 | 1 | 10110 | −0.025488 |
| | Abundance (Late | | | | | |
| | vs Early) | | | | | |
| CRISPRi | GD2 CAR | TRIM56 | 0.60903 | 1 | 11114 | −0.027877 |
| | Abundance (Late | | | | | |
| | vs Early) | | | | | |
| CRISPRi | GD2 CAR | PTEN | 0.67164 | 1 | 12122 | −0.42536 |
| | Abundance (Late | | | | | |
| | vs Early) | | | | | |
| CRISPRi | GD2 CAR | ATXN7L3 | 0.7214 | 1 | 12940 | −0.064123 |
| | Abundance (Late | | | | | |
| | vs Early) | | | | | |
| CRISPRi | GD2 CAR | SBNO2 | 0.7555 | 1 | 13532 | 0.11285 |
| | Abundance (Late | | | | | |
| | vs Early) | | | | | |
| CRISPRi | GD2 CAR | TNIP1 | 0.76876 | 1 | 13777 | −0.25136 |
| | Abundance (Late | | | | | |
| | vs Early) | | | | | |
| CRISPRi | GD2 CAR | GABARAPL1 | 0.81012 | 1 | 14484 | −0.59137 |
| | Abundance (Late | | | | | |
| | vs Early) | | | | | |
| CRISPRi | GD2 CAR | CUTA | 0.837 | 1 | 14940 | −0.4818 |
| | Abundance (Late | | | | | |
| | vs Early) | | | | | |
| CRISPRi | GD2 CAR | ARIH1 | 0.93026 | 1 | 16540 | −0.33117 |
| | Abundance (Late | | | | | |
| | vs Early) | | | | | |
| CRISPRi | GD2 CAR | ZBTB21 | 0.98245 | 1 | 17590 | −0.22637 |
| | Abundance (Late | | | | | |
| | vs Early) | | | | | |
| CRISPRi | CD19 CAR | CAND1 | 2.61E−07 | 7.07E−04 | 1 | 1.5189 |
| | Abundance (Late | | | | | |
| | vs Early) | | | | | |
| CRISPRi | CD19 CAR | ZMYND8 | 2.61E−07 | 7.07E−04 | 2 | 1.1731 |
| | Abundance (Late | | | | | |
| | vs Early) | | | | | |
| CRISPRi | CD19 CAR | MYB | 2.61E−07 | 7.07E−04 | 3 | 1.6399 |
| | Abundance (Late | | | | | |
| | vs Early) | | | | | |
| CRISPRi | CD19 CAR | PTEN | 2.61E−07 | 7.07E−04 | 4 | 0.89151 |
| | Abundance (Late | | | | | |
| | vs Early) | | | | | |
| CRISPRi | CD19 CAR | UBE2L3 | 2.61E−07 | 7.07E−04 | 5 | 1.3309 |
| | Abundance (Late | | | | | |
| | vs Early) | | | | | |
| CRISPRi | CD19 CAR | DCUN1D3 | 2.61E−07 | 7.07E−04 | 6 | 0.95612 |
| | Abundance (Late | | | | | |
| | vs Early) | | | | | |
| CRISPRi | CD19 CAR | MED24 | 2.61E−07 | 7.07E−04 | 7 | 1.1109 |
| | Abundance (Late | | | | | |
| | vs Early) | | | | | |
| CRISPRi | CD19 CAR | ATXN7L3 | 1.31E−06 | 0.003094 | 8 | 0.83806 |
| | Abundance (Late | | | | | |
| | vs Early) | | | | | |
| CRISPRi | CD19 CAR | RNF7 | 1.83E−06 | 0.00385 | 9 | 1.0459 |
| | Abundance (Late | | | | | |
| | vs Early) | | | | | |
| CRISPRi | CD19 CAR | MED23 | 2.88E−06 | 0.00495 | 10 | 0.68605 |
| | Abundance (Late | | | | | |
| | vs Early) | | | | | |
| CRISPRi | CD19 CAR | UBAP2L | 2.88E−06 | 0.00495 | 11 | 0.90127 |
| | Abundance (Late | | | | | |
| | vs Early) | | | | | |
| CRISPRi | CD19 CAR | CISH | 4.44E−06 | 0.007013 | 12 | 0.97991 |
| | Abundance (Late | | | | | |
| | vs Early) | | | | | |
| CRISPRi | CD19 CAR | ATF7IP | 4.97E−06 | 0.007235 | 13 | 0.82507 |
| | Abundance (Late | | | | | |
| | vs Early) | | | | | |
| CRISPRi | CD19 CAR | CDKN2A | 6.53E−06 | 0.00884 | 14 | 0.94598 |
| | Abundance (Late | | | | | |
| | vs Early) | | | | | |
| CRISPRi | CD19 CAR | RASA2 | 7.58E−06 | 0.009571 | 15 | 0.90347 |
| | Abundance (Late | | | | | |
| | vs Early) | | | | | |
| CRISPRi | CD19 CAR | UBE2F | 8.63E−06 | 0.010192 | 16 | 0.67293 |
| | Abundance (Late | | | | | |
| | vs Early) | | | | | |
| CRISPRi | CD19 CAR | COPS7B | 9.15E−06 | 0.010192 | 17 | 0.72803 |
| | Abundance (Late | | | | | |
| | vs Early) | | | | | |
| CRISPRi | CD19 CAR | KIAA1551 | 1.02E−05 | 0.010726 | 18 | 0.83951 |
| | Abundance (Late | | | | | |
| | vs Early) | | | | | |
| CRISPRi | CD19 CAR | RPS6KA1 | 1.23E−05 | 0.012246 | 19 | 0.86299 |
| | Abundance (Late | | | | | |
| | vs Early) | | | | | |
| CRISPRi | CD19 CAR | ARIH1 | 1.49E−05 | 0.014109 | 20 | 0.66062 |
| | Abundance (Late | | | | | |
| | vs Early) | | | | | |
| CRISPRi | CD19 CAR | TRIM56 | 3.53E−05 | 0.030378 | 21 | 0.69375 |
| | Abundance (Late | | | | | |
| | vs Early) | | | | | |
| CRISPRi | CD19 CAR | CUL5 | 3.53E−05 | 0.030378 | 22 | 1.0064 |
| | Abundance (Late | | | | | |
| | vs Early) | | | | | |
| CRISPRi | CD19 CAR | TNIP1 | 4.08E−05 | 0.033577 | 23 | 0.90498 |
| | Abundance (Late | | | | | |
| | vs Early) | | | | | |
| CRISPRi | CD19 CAR | PAK2 | 4.37E−05 | 0.034447 | 24 | 0.69053 |
| | Abundance (Late | | | | | |
| | vs Early) | | | | | |
| CRISPRi | CD19 CAR | TSPEAR | 4.57E−05 | 0.034653 | 25 | 0.80196 |
| | Abundance (Late | | | | | |
| | vs Early) | | | | | |
| CRISPRi | CD19 CAR | GABARAPL1 | 6.19E−05 | 0.045126 | 26 | 0.83669 |
| | Abundance (Late | | | | | |
| | vs Early) | | | | | |
| CRISPRi | CD19 CAR | SND1 | 6.93E−05 | 0.047914 | 27 | 0.62949 |
| | Abundance (Late | | | | | |
| | vs Early) | | | | | |
| CRISPRi | CD19 CAR | ZBTB21 | 7.08E−05 | 0.047914 | 28 | 0.84518 |
| | Abundance (Late | | | | | |
| | vs Early) | | | | | |
| CRISPRi | CD19 CAR | MYO5A | 7.97E−05 | 0.052066 | 29 | 0.20955 |
| | Abundance (Late | | | | | |
| | vs Early) | | | | | |
| CRISPRi | CD19 CAR | RNASE9 | 8.29E−05 | 0.05231 | 30 | 0.62712 |
| | Abundance (Late | | | | | |
| | vs Early) | | | | | |
| CRISPRi | CD19 CAR | CASP3 | 9.28E−05 | 0.055538 | 31 | 0.67113 |
| | Abundance (Late | | | | | |
| | vs Early) | | | | | |
| CRISPRi | CD19 CAR | WDFY1 | 9.38E−05 | 0.055538 | 32 | 0.71215 |
| | Abundance (Late | | | | | |
| | vs Early) | | | | | |
| CRISPRi | CD19 CAR | DDA1 | 9.96E−05 | 0.057156 | 33 | 0.86491 |
| | Abundance (Late | | | | | |
| | vs Early) | | | | | |
| CRISPRi | CD19 CAR | KDM5C | 1.07E−04 | 0.059843 | 34 | 0.62538 |
| | Abundance (Late | | | | | |
| | vs Early) | | | | | |
| CRISPRi | CD19 CAR | KRTAP10-9 | 1.24E−04 | 0.066144 | 35 | 0.6586 |
| | Abundance (Late | | | | | |
| | vs Early) | | | | | |
| CRISPRi | CD19 CAR | NFKBIA | 1.26E−04 | 0.066144 | 36 | 0.47723 |
| | Abundance (Late | | | | | |
| | vs Early) | | | | | |
| CRISPRi | CD19 CAR | CUTA | 1.37E−04 | 0.070377 | 37 | 0.26446 |
| | Abundance (Late | | | | | |
| | vs Early) | | | | | |
| CRISPRi | CD19 CAR | CPSF4L | 1.44E−04 | 0.071782 | 38 | 0.48111 |
| | Abundance (Late | | | | | |
| | vs Early) | | | | | |
| CRISPRi | CD19 CAR | BORCS8 | 1.52E−04 | 0.072153 | 39 | 0.59696 |
| | Abundance (Late | | | | | |
| | vs Early) | | | | | |
| CRISPRi | CD19 CAR | ARIH2 | 1.52E−04 | 0.072153 | 40 | 0.76093 |
| | Abundance (Late | | | | | |
| | vs Early) | | | | | |
| CRISPRi | CD19 CAR | ATF7IP2 | 1.60E−04 | 0.074016 | 41 | 0.48327 |
| | Abundance (Late | | | | | |
| | vs Early) | | | | | |
| CRISPRi | CD19 CAR | CFAP54 | 2.00E−04 | 0.087421 | 42 | 0.62074 |
| | Abundance (Late | | | | | |
| | vs Early) | | | | | |
| CRISPRi | CD19 CAR | SBNO2 | 2.01E−04 | 0.087421 | 43 | −0.020683 |
| | Abundance (Late | | | | | |
| | vs Early) | | | | | |
| CRISPRi | CD19 CAR | CDK13 | 2.03E−04 | 0.087421 | 44 | 0.78939 |
| | Abundance (Late | | | | | |
| | vs Early) | | | | | |
| CRISPRi | CD19 CAR | ST8SIA5 | 2.23E−04 | 0.094059 | 45 | 0.79382 |
| | Abundance (Late | | | | | |
| | vs Early) | | | | | |
| CRISPRi | CD19 CAR | MADD | 2.37E−04 | 0.097611 | 46 | 0.12178 |
| | Abundance (Late | | | | | |
| | vs Early) | | | | | |
| CRISPRi | CD19 CAR | UBE2E1 | 3.78E−04 | 0.140264 | 51 | 0.60988 |
| | Abundance (Late | | | | | |
| | vs Early) | | | | | |
| CRISPRi | CD19 CAR | ELOC | 0.0029491 | 0.419973 | 133 | 0.45709 |
| | Abundance (Late | | | | | |
| | vs Early) | | | | | |
| CRISPRi | CD19 CAR | ELOB | 0.054652 | 0.775903 | 1325 | 0.15537 |
| | Abundance (Late | | | | | |
| | vs Early) | | | | | |
| CRISPRi | CD19 CAR | MED13 | 0.065851 | 0.787657 | 1579 | 0.2954 |
| | Abundance (Late | | | | | |
| | vs Early) | | | | | |
| CRISPRi | CD19 CAR | SYCE1L | 0.085379 | 0.827767 | 1950 | 0.25724 |
| | Abundance (Late | | | | | |
| | vs Early) | | | | | |
| CRISPRi | CD19 CAR | SRRD | 0.17396 | 0.92009 | 3565 | 0.28341 |
| | Abundance (Late | | | | | |
| | vs Early) | | | | | |
| CRISPRi | CD19 CAR | SPIN1 | 0.29629 | 0.974907 | 5735 | 0.21204 |
| | Abundance (Late | | | | | |
| | vs Early) | | | | | |
| CRISPRi | CD19 CAR | ARHGAP5 | 0.38231 | 0.997751 | 7235 | −0.072974 |
| | Abundance (Late | | | | | |
| | vs Early) | | | | | |
| CRISPRi | CD19 CAR | SYTL2 | 0.59476 | 1 | 10833 | −0.17737 |
| | Abundance (Late | | | | | |
| | vs Early) | | | | | |
| CRISPRi | CD19 CAR | MEOX1 | 0.60232 | 1 | 10960 | −0.15922 |
| | Abundance (Late | | | | | |
| | vs Early) | | | | | |
| CRISPRi | CD19 CAR | IGFL4 | 0.67206 | 1 | 12106 | 0.092701 |
| | Abundance (Late | | | | | |
| | vs Early) | | | | | |
| CRISPRi | CD19 CAR | PRDM14 | 0.68781 | 1 | 12370 | 0.14768 |
| | Abundance (Late | | | | | |
| | vs Early) | | | | | |
| CRISPRi | CD19 CAR | NQO2 | 0.75395 | 1 | 13415 | −0.30445 |
| | Abundance (Late | | | | | |
| | vs Early) | | | | | |
| CRISPRi | CD19 CAR | MOCS3 | 0.79295 | 1 | 14063 | −0.64525 |
| | Abundance (Late | | | | | |
| | vs Early) | | | | | |
| CRISPRi | CD19 CAR | MED19 | 0.8201 | 1 | 14493 | −0.06066 |
| | Abundance (Late | | | | | |
| | vs Early) | | | | | |
| CRISPRi | CD19 CAR | MN1 | 0.82438 | 1 | 14566 | 0.017534 |
| | Abundance (Late | | | | | |
| | vs Early) | | | | | |
| CRISPRi | CD19 CAR | MED1 | 0.88941 | 1 | 15687 | −0.038942 |
| | Abundance (Late | | | | | |
| | vs Early) | | | | | |
| CRISPRi | CD19 CAR | PIK3R5 | 0.97192 | 1 | 17257 | −0.79911 |
| | Abundance (Late | | | | | |
| | vs Early) | | | | | |
| CRISPRi | CD19 CAR | MED12 | 0.98688 | 1 | 17593 | −0.54253 |
| | Abundance (Late | | | | | |
| | vs Early) | | | | | |
| CRISPRi | CD19 CAR | PDCL | 0.99193 | 1 | 17741 | −0.652 |
| | Abundance (Late | | | | | |
| | vs Early) | | | | | |
| CRISPRi | CD19 CAR | CCNC | 0.99195 | 1 | 17742 | −0.36683 |
| | Abundance (Late | | | | | |
| | vs Early) | | | | | |
| CRISPRi | CD19 CAR | CBFB | 0.99209 | 1 | 17746 | −1.6667 |
| | Abundance (Late | | | | | |
| | vs Early) | | | | | |
| CRISPRi | CD19 CAR | MAPKAP1 | 0.99999 | 1 | 18515 | −1.2037 |
| | Abundance (Late | | | | | |
| | vs Early) | | | | | |
| CRISPRi | GD2 CAR vs CD19 | MED12 | 2.61E−07 | 0.001238 | 1 | 3.8456 |
| | CAR (Late | | | | | |
| | Timepoints) | | | | | |
| CRISPRi | GD2 CAR vs CD19 | PDCL | 2.61E−07 | 0.001238 | 2 | 1.7441 |
| | CAR (Late | | | | | |
| | Timepoints) | | | | | |
| CRISPRi | GD2 CAR vs CD19 | CCNC | 2.61E−07 | 0.001238 | 3 | 2.1345 |
| | CAR (Late | | | | | |
| | Timepoints) | | | | | |
| CRISPRi | GD2 CAR vs CD19 | CBFB | 2.61E−07 | 0.001238 | 4 | 2.3323 |
| | CAR (Late | | | | | |
| | Timepoints) | | | | | |
| CRISPRi | GD2 CAR vs CD19 | PIK3R5 | 2.88E−06 | 0.010891 | 5 | 1.294 |
| | CAR (Late | | | | | |
| | Timepoints) | | | | | |
| CRISPRi | GD2 CAR vs CD19 | MED19 | 5.49E−06 | 0.017327 | 6 | 1.3695 |
| | CAR (Late | | | | | |
| | Timepoints) | | | | | |
| CRISPRi | GD2 CAR vs CD19 | MAPKAP1 | 1.02E−05 | 0.024752 | 7 | 1.868 |
| | CAR (Late | | | | | |
| | Timepoints) | | | | | |
| CRISPRi | GD2 CAR vs CD19 | NQO2 | 1.18E−05 | 0.024752 | 8 | 1.0874 |
| | CAR (Late | | | | | |
| | Timepoints) | | | | | |
| CRISPRi | GD2 CAR vs CD19 | ARHGAP5 | 1.18E−05 | 0.024752 | 9 | 1.1095 |
| | CAR (Late | | | | | |
| | Timepoints) | | | | | |
| CRISPRi | GD2 CAR vs CD19 | ELOB | 1.59E−05 | 0.030153 | 10 | 1.5391 |
| | CAR (Late | | | | | |
| | Timepoints) | | | | | |
| CRISPRi | GD2 CAR vs CD19 | MOCS3 | 1.75E−05 | 0.030153 | 11 | 0.53863 |
| | CAR (Late | | | | | |
| | Timepoints) | | | | | |
| CRISPRi | GD2 CAR vs CD19 | MED13 | 2.01E−05 | 0.031766 | 12 | 0.84099 |
| | CAR (Late | | | | | |
| | Timepoints) | | | | | |
| CRISPRi | GD2 CAR vs CD19 | SRRD | 7.24E−05 | 0.105021 | 13 | 1.1248 |
| | CAR (Late | | | | | |
| | Timepoints) | | | | | |
| CRISPRi | GD2 CAR vs CD19 | PRDM14 | 7.76E−05 | 0.105021 | 14 | 1.0395 |
| | CAR (Late | | | | | |
| | Timepoints) | | | | | |
| CRISPRi | GD2 CAR vs CD19 | ELOC | 1.05E−04 | 0.132343 | 15 | 0.79173 |
| | CAR (Late | | | | | |
| | Timepoints) | | | | | |
| CRISPRi | GD2 CAR vs CD19 | MN1 | 1.21E−04 | 0.134246 | 17 | 0.84276 |
| | CAR (Late | | | | | |
| | Timepoints) | | | | | |
| CRISPRi | GD2 CAR vs CD19 | MED24 | 2.06E−04 | 0.200701 | 19 | 1.0934 |
| | CAR (Late | | | | | |
| | Timepoints) | | | | | |
| CRISPRi | GD2 CAR vs CD19 | MEOX1 | 2.54E−04 | 0.200701 | 23 | 1.2491 |
| | CAR (Late | | | | | |
| | Timepoints) | | | | | |
| CRISPRi | GD2 CAR vs CD19 | SYCE1L | 3.33E−04 | 0.242765 | 26 | 0.76905 |
| | CAR (Late | | | | | |
| | Timepoints) | | | | | |
| CRISPRi | GD2 CAR vs CD19 | SYTL2 | 6.26E−04 | 0.305603 | 37 | 0.80483 |
| | CAR (Late | | | | | |
| | Timepoints) | | | | | |
| CRISPRi | GD2 CAR vs CD19 | RNF7 | 7.10E−04 | 0.308416 | 42 | 0.78538 |
| | CAR (Late | | | | | |
| | Timepoints) | | | | | |
| CRISPRi | GD2 CAR vs CD19 | ATF7IP2 | 7.13E−04 | 0.308416 | 43 | 0.88511 |
| | CAR (Late | | | | | |
| | Timepoints) | | | | | |
| CRISPRi | GD2 CAR vs CD19 | UBE2E1 | 9.41E−04 | 0.342631 | 52 | 0.86235 |
| | CAR (Late | | | | | |
| | Timepoints) | | | | | |
| CRISPRi | GD2 CAR vs CD19 | MED1 | 0.0024786 | 0.469455 | 100 | 1.1692 |
| | CAR (Late | | | | | |
| | Timepoints) | | | | | |
| CRISPRi | GD2 CAR vs CD19 | SPIN1 | 0.0025215 | 0.472846 | 101 | 0.9936 |
| | CAR (Late | | | | | |
| | Timepoints) | | | | | |
| CRISPRi | GD2 CAR vs CD19 | COPS7B | 0.020136 | 0.792396 | 480 | 0.22539 |
| | CAR (Late | | | | | |
| | Timepoints) | | | | | |
| CRISPRi | GD2 CAR vs CD19 | IGFL4 | 0.021886 | 0.794536 | 508 | 0.54593 |
| | CAR (Late | | | | | |
| | Timepoints) | | | | | |
| CRISPRi | GD2 CAR vs CD19 | UBAP2L | 0.041563 | 0.868392 | 903 | 0.24222 |
| | CAR (Late | | | | | |
| | Timepoints) | | | | | |
| CRISPRi | GD2 CAR vs CD19 | NFKBIA | 0.12102 | 0.976748 | 2320 | −0.30086 |
| | CAR (Late | | | | | |
| | Timepoints) | | | | | |
| CRISPRi | GD2 CAR vs CD19 | KIAA1551 | 0.167 | 1 | 3136 | 0.29632 |
| | CAR (Late | | | | | |
| | Timepoints) | | | | | |
| CRISPRi | GD2 CAR vs CD19 | DCUN1D3 | 0.18177 | 1 | 3408 | −0.019604 |
| | CAR (Late | | | | | |
| | Timepoints) | | | | | |
| CRISPRi | GD2 CAR vs CD19 | CASP3 | 0.21645 | 1 | 4030 | −0.055236 |
| | CAR (Late | | | | | |
| | Timepoints) | | | | | |
| CRISPRi | GD2 CAR vs CD19 | CAND1 | 0.24663 | 1 | 4601 | −0.096495 |
| | CAR (Late | | | | | |
| | Timepoints) | | | | | |
| CRISPRi | GD2 CAR vs CD19 | SND1 | 0.29028 | 1 | 5348 | −0.17049 |
| | CAR (Late | | | | | |
| | Timepoints) | | | | | |
| CRISPRi | GD2 CAR vs CD19 | MADD | 0.30453 | 1 | 5583 | −0.30758 |
| | CAR (Late | | | | | |
| | Timepoints) | | | | | |
| CRISPRi | GD2 CAR vs CD19 | CFAP54 | 0.30534 | 1 | 5596 | −0.026544 |
| | CAR (Late | | | | | |
| | Timepoints) | | | | | |
| CRISPRi | GD2 CAR vs CD19 | KRTAP10-9 | 0.38288 | 1 | 7098 | −0.17569 |
| | CAR (Late | | | | | |
| | Timepoints) | | | | | |
| CRISPRi | GD2 CAR vs CD19 | ARIH2 | 0.45708 | 1 | 8467 | 0.090844 |
| | CAR (Late | | | | | |
| | Timepoints) | | | | | |
| CRISPRi | GD2 CAR vs CD19 | MYO5A | 0.46425 | 1 | 8619 | −0.90991 |
| | CAR (Late | | | | | |
| | Timepoints) | | | | | |
| CRISPRi | GD2 CAR vs CD19 | TSPEAR | 0.4671 | 1 | 8665 | 0.0055466 |
| | CAR (Late | | | | | |
| | Timepoints) | | | | | |
| CRISPRi | GD2 CAR vs CD19 | BORCS8 | 0.51205 | 1 | 9451 | −0.51234 |
| | CAR (Late | | | | | |
| | Timepoints) | | | | | |
| CRISPRi | GD2 CAR vs CD19 | CUL5 | 0.51923 | 1 | 9585 | −0.62277 |
| | CAR (Late | | | | | |
| | Timepoints) | | | | | |
| CRISPRi | GD2 CAR vs CD19 | PAK2 | 0.53914 | 1 | 9965 | −0.73379 |
| | CAR (Late | | | | | |
| | Timepoints) | | | | | |
| CRISPRi | GD2 CAR vs CD19 | UBE2L3 | 0.55091 | 1 | 10193 | −0.65651 |
| | CAR (Late | | | | | |
| | Timepoints) | | | | | |
| CRISPRi | GD2 CAR vs CD19 | RNASE9 | 0.6069 | 1 | 11234 | −0.3885 |
| | CAR (Late | | | | | |
| | Timepoints) | | | | | |
| CRISPRi | GD2 CAR vs CD19 | ATXN7L3 | 0.72668 | 1 | 13463 | −0.85109 |
| | CAR (Late | | | | | |
| | Timepoints) | | | | | |
| CRISPRi | GD2 CAR vs CD19 | WDFY1 | 0.72844 | 1 | 13494 | −0.37572 |
| | CAR (Late | | | | | |
| | Timepoints) | | | | | |
| CRISPRi | GD2 CAR vs CD19 | MED23 | 0.76257 | 1 | 14124 | −0.16467 |
| | CAR (Late | | | | | |
| | Timepoints) | | | | | |
| CRISPRi | GD2 CAR vs CD19 | UBE2F | 0.76945 | 1 | 14267 | −0.17933 |
| | CAR (Late | | | | | |
| | Timepoints) | | | | | |
| CRISPRi | GD2 CAR vs CD19 | GABARAPL1 | 0.79416 | 1 | 14796 | −1.2938 |
| | CAR (Late | | | | | |
| | Timepoints) | | | | | |
| CRISPRi | GD2 CAR vs CD19 | CPSF4L | 0.79703 | 1 | 14854 | −0.50705 |
| | CAR (Late | | | | | |
| | Timepoints) | | | | | |
| CRISPRi | GD2 CAR vs CD19 | TRIM56 | 0.81873 | 1 | 15294 | −0.4826 |
| | CAR (Late | | | | | |
| | Timepoints) | | | | | |
| CRISPRi | GD2 CAR vs CD19 | CDKN2A | 0.8497 | 1 | 15863 | −0.36384 |
| | CAR (Late | | | | | |
| | Timepoints) | | | | | |
| CRISPRi | GD2 CAR vs CD19 | DDA1 | 0.85954 | 1 | 16055 | −0.24472 |
| | CAR (Late | | | | | |
| | Timepoints) | | | | | |
| CRISPRi | GD2 CAR vs CD19 | CUTA | 0.88333 | 1 | 16558 | −0.6789 |
| | CAR (Late | | | | | |
| | Timepoints) | | | | | |
| CRISPRi | GD2 CAR vs CD19 | CISH | 0.88963 | 1 | 16652 | −0.61431 |
| | CAR (Late | | | | | |
| | Timepoints) | | | | | |
| CRISPRi | GD2 CAR vs CD19 | RPS6KA1 | 0.89691 | 1 | 16800 | −0.20772 |
| | CAR (Late | | | | | |
| | Timepoints) | | | | | |
| CRISPRi | GD2 CAR vs CD19 | ZMYND8 | 0.90296 | 1 | 16907 | −0.35354 |
| | CAR (Late | | | | | |
| | Timepoints) | | | | | |
| CRISPRi | GD2 CAR vs CD19 | KDM5C | 0.91459 | 1 | 17114 | −0.12989 |
| | CAR (Late | | | | | |
| | Timepoints) | | | | | |
| CRISPRi | GD2 CAR vs CD19 | PTEN | 0.93728 | 1 | 17552 | −1.3982 |
| | CAR (Late | | | | | |
| | Timepoints) | | | | | |
| CRISPRi | GD2 CAR vs CD19 | SBNO2 | 0.94019 | 1 | 17617 | −0.59561 |
| | CAR (Late | | | | | |
| | Timepoints) | | | | | |
| CRISPRi | GD2 CAR vs CD19 | CDK13 | 0.94234 | 1 | 17653 | −0.43648 |
| | CAR (Late | | | | | |
| | Timepoints) | | | | | |
| CRISPRi | GD2 CAR vs CD19 | TNIP1 | 0.95147 | 1 | 17832 | −1.2316 |
| | CAR (Late | | | | | |
| | Timepoints) | | | | | |
| CRISPRi | GD2 CAR vs CD19 | ATF7IP | 0.96582 | 1 | 18143 | −0.4039 |
| | CAR (Late | | | | | |
| | Timepoints) | | | | | |
| CRISPRi | GD2 CAR vs CD19 | RASA2 | 0.96819 | 1 | 18193 | −1.2981 |
| | CAR (Late | | | | | |
| | Timepoints) | | | | | |
| CRISPRi | GD2 CAR vs CD19 | MYB | 0.98898 | 1 | 18600 | −1.2852 |
| | CAR (Late | | | | | |
| | Timepoints) | | | | | |
| CRISPRi | GD2 CAR vs CD19 | ST8SIA5 | 0.99283 | 1 | 18710 | −0.55014 |
| | CAR (Late | | | | | |
| | Timepoints) | | | | | |
| CRISPRi | GD2 CAR vs CD19 | ZBTB21 | 0.9988 | 1 | 18886 | −1.1292 |
| | CAR (Late | | | | | |
| | Timepoints) | | | | | |
| CRISPRi | GD2 CAR vs CD19 | ARIH1 | 0.99984 | 1 | 18925 | −0.65672 |
| | CAR (Late | | | | | |
| | Timepoints) | | | | | |

### Example 2

Nine hits of interest from the CRISPRa screens were cloned as individual sgRNA vectors to perform arrayed in vitro cancer cell killing assays. The hits/sgRNAs improved cancer cell killing (including target-cell dependent), which demonstrates their usefulness in a T cell immunotherapy.

### Methods

CD8+ T cells were cultured in X-VIVO 15 (Lonza Bioscience, catalog no. 04-418Q) supplemented with 5% fetal calf serum (FCS), and 100 IU/ml of recombinant human IL-2 (Amerisource Bergen, catalog no. 10101641). Primary CD8+ T cells were activated using anti-human CD3/CD28 CTS Dynabeads (Fisher Scientific, catalog no. 40203D) at a 1:1 cell: bead ratio at 106 cells/ml. Eighteen hours after activation, CD8+ T cells from two human blood donors were transduced with concentrated dCas9-2A-BlasticidinResistance. Eight hours following dCas9 transduction, cells were split into two flasks and transduced with CAR lentiviruses, anti-CD19-28-zeta-2A-GFP, or high-affinity (HA) anti-GD2-28-zeta-2A-GFP. 16 hours following CAR transductions, cells were split into 20 wells and transduced with CRISPRa sgRNA lentiviruses targeting 9 hits from CRISPRa screens (2 sgRNAs per gene hit) plus two no-target control sgRNAs. Three days post-activation puromycin and blasticidin was added to select for dCas9 and sgRNA transduced cells. Cells were passaged every other day, adding IL-2 to 100U/ml at each passage.

For the NALM-6-GD2 killing assay, CRISPRa CD19 or GD2 CAR T cells for each sgRNA were mixed with NALM-6 lymphoma target cells engineered to express GD2 at effector:target ratios ranging from 1:1 to 1:16 with 100U/ml IL-2. Cells were passaged 48 hr later, replenishing IL-2. 6-days after starting the killing assay, cells were stained with anti-CD8a (T cell marker) and anti-CD22 (NALM6 marker) and cell counts were measured by flow cytometry to determine NALM6-GD2 abundance compared to a no T cell control.

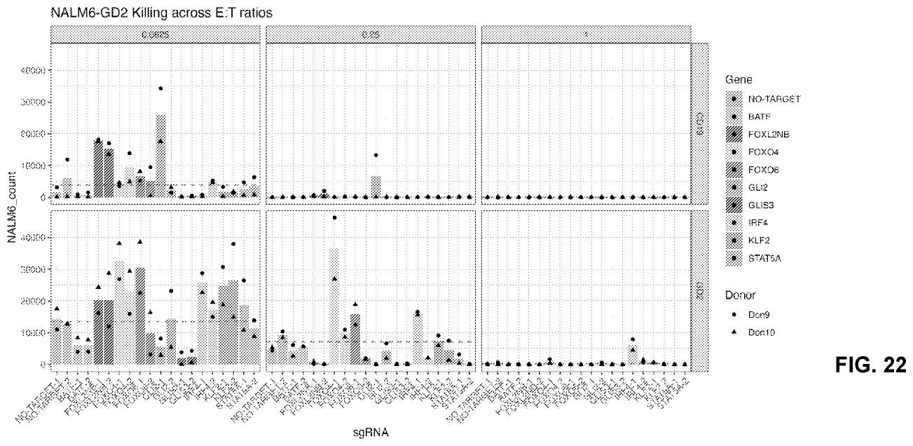

For the 143B killing assay, 143B cells (GD2+ osteosarcoma cell line) engineered to express nuclear RFP were seeded in 384-well plates 24-hours before adding T cells. 12-days post activation CRISPRa GD2 CAR T cells for each sgRNA were added to wells at effector:target ratios ranging from 9:1 to 1:27. 143B cell counts were measured with RFP fluorescence over time using an Incucyte instrument. Data presented in FIGS. 22-27.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A modified T cell, lymphoid cell or myeloid cell comprising a modification of one of more genes selected from BATF, CUL3, CLASRP, CDKL3, EOMES, FOXL2NB, FOXO6, FOXO4, FOSB, FOSL1, FOXL2, GLIS3, GL2, IRF4, IL2RA, INPP5D, IL2RB, ITPKA, ITPKB, HL10, IL9R, LIFR, LCK, LYN, NPM3, NQO1, PLEKHH3, PTEN, RFLNB, STAT5A, SHOC2, S100A13, TRAF3IP2, TMEM167B, TSR2, VAV1 or ZNF211, wherein the modification upregulates or increases expression of said gene in said modified T cell, lymphoid cell or myeloid cell as compared to a non-modified T cell, lymphoid cell or myeloid cell control.

2. The modified T cell, lymphoid cell or myeloid cell of claim 1, wherein the modification is one or more deletion, substitution or insertion into one or more of said genes.

3. The modified T cell, lymphoid cell or myeloid cell of claim 1, wherein the increased expression is by modification of one or more endogenous promoters of one or more of said genes.

4. The modified T cell, lymphoid cell or myeloid cell of claim 1, wherein the modified T cell, lymphoid cell or myeloid cell comprises one or more expression cassettes comprising a promoter operably linked to a nucleic acid segment comprising a coding region of any of said genes or a targeted knock-in of a cassette under the control of a selected endogenous promoter/gene regulatory element.

5. A modified T cell, lymphoid cell or myeloid cell comprising a modification of one of more of genes selected from CCNC, ELOB, ELOC, IGFL4, MED1, MED12 MED13, MN1, MED19, MEOX1, MOCS3, NQO2, PRDM14, SYCE1L, SYTL2, SRRD, SPIN1, UBF2E1, ARHGAP5, ARIH1, ARIH2, ATF7IP, ATF7IP2, ATXN7L3, BORCS8, CAND1, CASP3, CBFB, CDK13, CDKN2A, CFAP54, CISH, COPS7B, CPSF4L, CUL5, CUTA, DCUN1D3, DDA1, GABARAPL1, KDM5C, KIAA1551, KRTAP10-9, MADD, MAPKAP1, MED23, MED24, MYB, MYO5A, NFKBIA, PAK2, PDCL, PIK3R5, PTEN, RASA2, RNASE9, RNF7, RPS6KAI, SBNO2, SND1, ST8SIA5, TNIP1, TRIM56, TSPEAR, UBAP2L, UBE2F, UBE2L3, WDFY1, ZBTB21 or ZMYND8, wherein the modification downregulates or decreases expression of said gene in said modified T cell, lymphoid cell or myeloid cell as compared to a non-modified T cell, lymphoid cell or myeloid cell control.

6. The modified T cell, lymphoid cell or myeloid cell of claim 5, wherein the modified T cell, lymphoid cell or myeloid cell comprises an exogenous inhibitory nucleic acid (e.g., RNAi, shRNA, siRNA).

7. A method comprising ex vivo modification of any of the genes listed in Table 1 within at least one T cell, lymphoid cell or myeloid cell, or a combination thereof to generate at least one modified T cell, lymphoid cell, myeloid cell, or a mixture of modified T cells, lymphoid cells and myeloid cells.

8. The method of claim 7, wherein the modification is one or more deletion, substitution or insertion into one or more endogenous genomic sites of any of the genes listed in Table 1.

9. The method of claim 7, wherein the modification is reduction of expression or translation of any of the genes listed in Table 1.

10. The method of claim 9, wherein the reduction of expression or translation is by an inhibitory nucleic acid (e.g., RNAi, shRNA, siRNA).

11. The method of claim 7, wherein the modification is increased expression of any of the genes listed in Table 1.

12. The method of claim 11, wherein the increased expression is by modification of one or more promoters of any of the genes listed in Table 1.

13. The method of claim 7, wherein the modification is one or more CRISPR-mediated modifications or activations of any of the genes listed in Table 1.

14. The method of claim 7, wherein the modification is transformation of at least one T cell, lymphoid cell or myeloid cell, or a combination thereof, with one or more expression cassettes comprising a promoter operably linked to a nucleic acid segment comprising a coding region of any of the genes listed in Table 1 or a targeted knock-in of a cassette under the control of a selected endogenous promoter/gene regulatory element.

15. The method of claim 7, further comprising administering at least one modified T cell, lymphoid cell, myeloid cell, or a mixture of modified T cells, lymphoid cells and myeloid cells to a subject.

16. The method of claim 7, further comprising incubating the at least one modified T cell, lymphoid cell, myeloid cell, or a mixture of modified T cells, lymphoid cells and myeloid cells to form a population of modified cells.

17. The method of claim 16, further comprising administering the population of modified cells to a subject.

18. The method of claim 15, wherein the subject has a disease or condition.

19. The method of claim 18, wherein the disease or condition is an immune condition or cancer.

20.-24. (canceled)

25. A method to overcome T cell exhaustion comprising upregulating one or more genes selected from BATF, CUL3, CLASRP, CDKL3, EOMES, FOXL2NB, FOXO6, FOXO4, FOSB, FOSL1, FOXL2, GLIS3, GLI2, IRF4, IL2RA, INPP5D, IL2RB, ITPKA, ITPKB, IL10, IL9R, LIFR, LCK, LYN, NPM3, NQO1, PLEKHH3, PTEN, RFLNB, STAT5A, SHOC2, S100A13, TRAF3IP2, TMEM167B, TSR2, VAV1 or ZNF211 or downregulating one or more genes selected from CCNC, ELOB ELOC, IGFL4, MED1, MED12, MED13, MN1, MED19, MEOX1, MOCS3, NQO2, PRDM14, SYCE1L, SYTL2, SRRD, SPIN1, UBE2E1, ARHGAP5, ARIH1, ARIH2, ATF7JP, ATF7IP2, ATXN7L3, BORCS8, CAND1, CASP3, CBFB, CDK13, CDKN2A, CFAP54, CISH, COPS7B, CPSF4L, CUL5, CUTA, DCUN1D3, DDA1, GABARAPL1, KDM5C, KIAA1551, KRTAP10-9, MADD, MAPKAP1, MED23, MED24, MYB, MYO5A, NFKBIA, PAK2, PDCL, PIK3R5, PTEN, RASA2, RNASE9, RNF7, RPS6KA1, SBNO2, SND1, ST8SIA5, TNIP1, TRIM56, TSPEAR, UBAP2L, UBE2F, UBF2L3, WDFY1, ZBTB21 or ZMYND8 in a T cell, lymphoid cell, myeloid cell.
